(19) United States
(12) Patent Application Publication
(10) Pub. No.: US 2025/0255176 A1
(43) Pub. Date: Aug. 7, 2025

(54) HETEROCYCLIC COMPOUND, ORGANIC LIGHT-EMITTING ELEMENT COMPRISING SAME, AND COMPOSITION FOR ORGANIC LAYER OF ORGANIC LIGHT-EMITTING ELEMENT

(71) Applicant: LT MATERIALS CO.,LTD., Yongin-si (KR)

(72) Inventors: Gi-Back LEE, Yongin-si (KR); Jun-Tae MO, Yongin-si (KR); Dong-Jun KIM, Yongin-si (KR); Yong-Woo KIM, Yongin-si (KR); Dae-Hyuk CHOI, Yongin-si (KR)

(21) Appl. No.: 19/185,137

(22) Filed: Apr. 21, 2025

Related U.S. Application Data
(63) Continuation of application No. PCT/KR2023/013236, filed on Sep. 5, 2023.

(30) Foreign Application Priority Data
Oct. 24, 2022 (KR) 10-2022-0137100

(51) Int. Cl. H10K 85/60; C07D 239/26; C07D 251/24; C07D 405/04; C07D 405/10; C07D 405/14; C07D 409/14; C07D 491/048; C07D 495/04; C09K 11/02; H10K 50/11; H10K 101/00

(52) U.S. Cl. CPC H10K 85/615; C07D 239/26; C07D 251/24; C07D 405/04; C07D 405/10; C07D 405/14; C07D 409/14; C07D 491/048; C07D 495/04; C09K 11/02; H10K 85/622; H10K 85/633; H10K 85/636; H10K 50/11; H10K 85/626; H10K 85/654; H10K 85/6574; H10K 85/6576; H10K 2101/90

(57) ABSTRACT

Provided is a heterocyclic compound, an organic light emitting device including the same, and a composition for an organic material layer of the organic light emitting device. The heterocyclic compound described herein may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a light emitting layer of an organic light emitting device.

### CROSS-REFERENCE TO RELATED APPLICATIONS

This application claims priority under 35 U.S.C. 119(a) to KR 10-2022-0137100, filed on Oct. 24, 2022 in the Republic of Korea, the content of which is hereby expressly incorporated by reference in its entirety.

### TECHNICAL FIELD

The present invention relates to a heterocyclic compound, an organic light emitting device including the same, and a composition for an organic material layer of the organic light emitting device.

### BACKGROUND ART

An electroluminescence device is a kind of self-emitting type display device, and has an advantage in that the viewing angle is wide, the contrast is excellent, and the response speed is fast.

An organic light emitting device is composed of a structure in which an organic thin film is disposed between two electrodes. When a voltage is applied to an organic light emitting device having such a structure, electrons and holes injected from the two electrodes are combined with each other in the organic thin film to make a pair, and then, the paired electrons and holes emit light while being annihilated. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, efficiency and service life of the organic light emitting device, there is a continuous need for developing a material for an organic thin film.

### SUMMARY OF THE INVENTION

The present invention has been made in an effort to provide a heterocyclic compound, an organic light emitting device including the same, and a composition for an organic material layer of the organic light emitting device.

In an exemplary embodiment of the present application, provided is a heterocyclic compound represented by the following Chemical Formula 1.

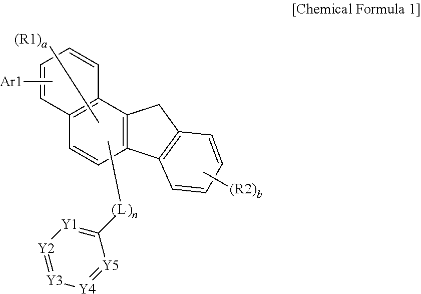

In Chemical Formula 1,
X is O, S or —C(Ra)(Rb), and Ra and Rb are the same as or different from each other, and are each independently a substituted or unsubstituted C1 to C60 alkyl group or a substituted or unsubstituted C6 to C60 aryl group,
   L is a direct bond; or a substituted or unsubstituted C6 to C60 arylene group,
n is an integer from 1 to 3, and when n is 2 or higher, substituents in the parenthesis are the same as or different from each other,
Y1 is CR11 or N, Y2 is CR12 or N, Y3 is CR13 or N, Y4 is CR14 or N, Y5 is CR15 or N, and at least one of Y1 to Y5 is N,
R11 to R15 are the same as or different from each other, and are each independently hydrogen; deuterium; a substituted or unsubstituted C6 to C60 aryl group; or a substituted or unsubstituted C2 to C60 heteroaryl group, or two or more adjacent groups are bonded to each other to form a substituted or unsubstituted C2 to C60 hetero ring,
Ar1 is a substituted or unsubstituted C6 to C60 aryl group; or a substituted or unsubstituted C2 to C60 heteroaryl group,
R1 and R2 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or two or more adjacent groups are bonded to each other to form a substituted or unsubstituted aliphatic or aromatic hydrocarbon ring or hetero ring,
a is an integer from 0 to 4, and when a is 2 or higher, substituents in the parenthesis are the same as or different from each other, and
b is an integer from 0 to 4, and when b is 2 or higher, substituents in the parenthesis are the same as or different from each other.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In addition, in an exemplary embodiment of the present application, provided is an organic light emitting device in which the organic material layer further includes a heterocyclic compound represented by the following Chemical Formula 2.

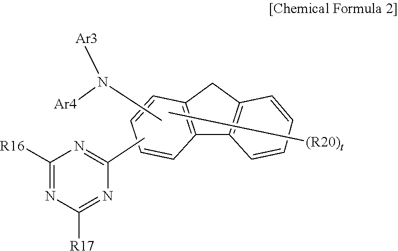

In Chemical Formula 2,
X1 is O or S,
R20 is hydrogen; deuterium; a halogen; a cyano group; a substituted or unsubstituted C1 to C60 alkyl group; a substituted or unsubstituted C6 to C60 aryl group; or a substituted or unsubstituted C2 to C60 heteroaryl group,
t is an integer from 1 to 4, and when t is 2 or higher, substituents in the parenthesis are the same as or different from each other,
Ar3 and Ar4 are the same as or different from each other, and are each independently a substituted or unsubstituted C6 to C60 aryl group; or a substituted or unsubstituted C2 to C60 heteroaryl group, and
R16 and R17 are the same as or different from each other, and are each independently a substituted or unsubstituted C6 to C60 aryl group; or a substituted or unsubstituted C2 to C60 heteroaryl group.

Further, in an exemplary embodiment of the present application, provided is a composition for an organic material layer of an organic light emitting device, which includes the heterocyclic compound represented by Chemical Formula 1; and the heterocyclic compound represented by Chemical Formula 2.

### Advantageous Effects

The heterocyclic compound described in the present specification may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a light emitting layer of an organic light emitting device.

Specifically, one or two or more of the heterocyclic compounds represented by Chemical Formula 1 can be used, and the heterocyclic compounds represented by Chemical Formula 1 can be used as materials for the light emitting layer. In particular, the heterocyclic compound can be used as a host material for the light emitting layer of an organic light emitting device by introducing various substituents and changing the binding position of the substituent to adjust the bandgap.

The heterocyclic compound of the present invention has an advantage in that structural stability is better than a mono-substituted structure or a tri-substituted structure and hole mobility is also higher than that of a mono- or tri-substituted structure by having a di-substituted structure in which (1) an azine-based substituent and (2) an aryl group or heteroaryl group are introduced into the core structure thereof and simultaneously binding the above-described substituents in (1) and (2) to specific positions.

In particular, when the heterocyclic compound of the present invention is used in the light emitting layer of an organic light emitting device, it is possible to obtain an effect in which the driving voltage of the device is lowered, the efficiency of the device is also increased, and the service life of the device is extended.

### BRIEF DESCRIPTION OF THE DRAWINGS

FIGS. 1 to 4 are views each schematically illustrating a stacking structure of an organic light emitting device according to an exemplary embodiment of the present application.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
307: Electron blocking layer
400: Negative electrode

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification,

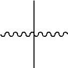

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, a deuterium content of 20% in a phenyl group represented by

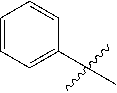 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

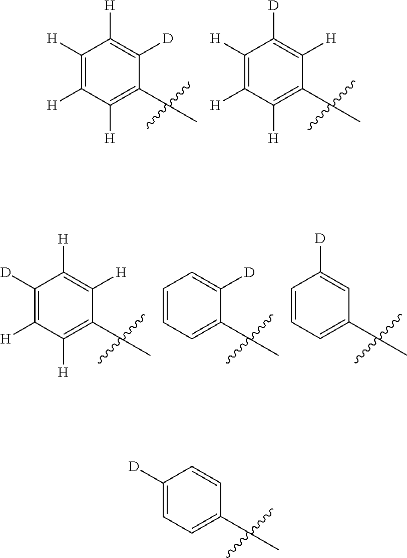

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a i-methyl-butyl group, a i-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes 0, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. H ere, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

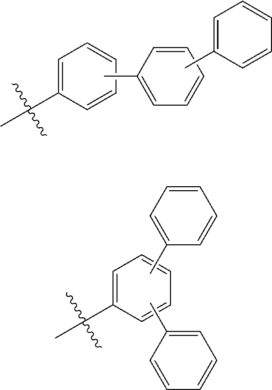

In the present specification, the fluorenyl group may be substituted, and adjacent substituents may be bonded to each other to form a ring.

When the fluorenyl group is substituted, the substituent may be

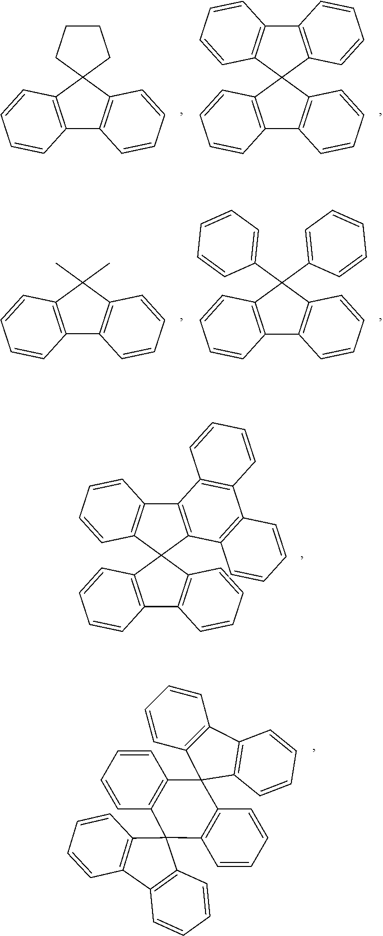

and the like, but is not limited thereto.

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole) group, a dihydrophenazine group, a phenoxazine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

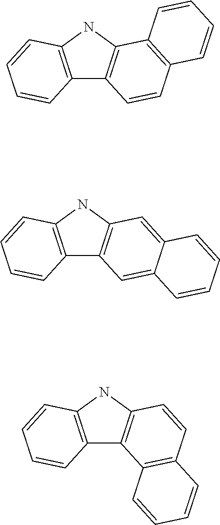 [SMILES: c1ccc2c(c1)ccc1c3ccccc3[nH]c21 ; c1ccc2cc3c(cc2c1)[nH]c1ccccc13 ; c1ccc2c(c1)ccc1[nH]c3ccccc3c12]

In the present specification, a dibenzocarbazole group may be any one of the following structures.

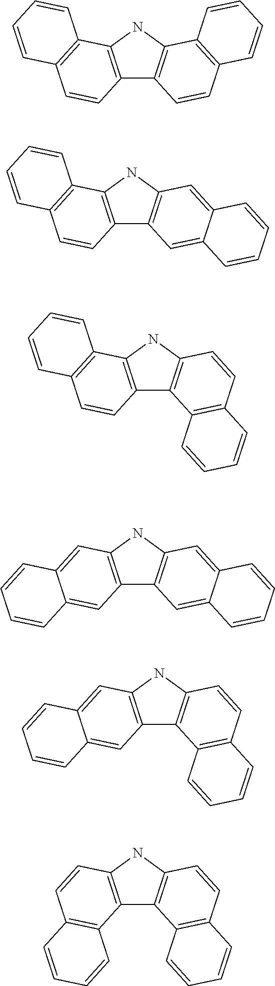 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, a naphthobenzofuran group may be any one of the following structures.

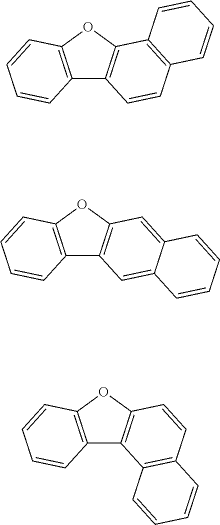 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

In the present specification, a naphthobenzothiophene group may be any one of the following structures.

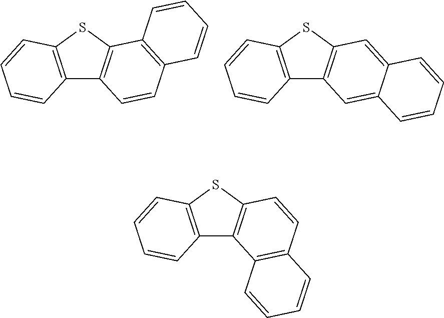

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

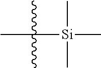 [SMILES: *[Si](C)(C)C]

(a trimethylsilyl group),

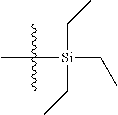 [SMILES: *[Si](CC)(CC)CC]

(a triethylsilyl group),

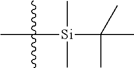 [SMILES: *[Si](C)(C)C(C)(C)C]

(a t-butyldimethylsilyl group),

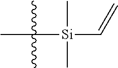 [SMILES: *[Si](C)(C)C=C]

(a vinyldimethylsilyl group),

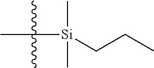 [SMILES: *[Si](C)(C)CCC]

(a propyldimethylsilyl group),

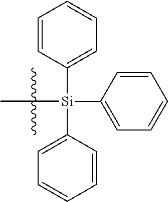 [SMILES: *[Si](c1ccccc1)(c1ccccc1)c1ccccc1]

(a triphenylsilyl group),

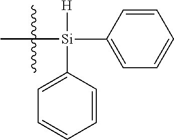 [SMILES: *[SiH](c1ccccc1)c1ccccc1]

(a diphenylsilyl group),

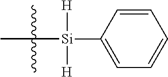 [SMILES: *[SiH2]c1ccccc1]

(a phenylsilyl group) and the like, but are not limited thereto.

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

In an exemplary embodiment of the present application, provided is the compound represented by Chemical Formula 1.

In an exemplary embodiment of the present application, a group not represented by a substituent; or a group represented by hydrogen may mean being all substitutable with deuterium. That is, it may be shown that hydrogen; or deuterium can be substituted with each other.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy.

Further, the single bond dissociation energy of carbon and deuterium is higher than the single bond dissociation energy of carbon and hydrogen. Accordingly, the deuterium-substituted structure has an effect of increasing the thermal stability of the molecule and improving the service life of the device using the increased thermal stability.

When a compound is deposited on a silicon wafer, a material including deuterium tends to be packed so that the intermolecular distance is reduced. Further, when the surface of a thin film is observed using an atomic force microscope (AFM), it can be confirmed that the thin film made of a compound including deuterium is deposited with a more uniform surface without any aggregated portion.

The heterocyclic compound of Chemical Formula 1 of the present application has a deuterium substitution rate of more than 0% and 100% or less. The deuterium-substituted compound is characterized in that the energy in the ground state is further lower than that of the hydrogen-substituted compound, and the shorter the bond length between carbon and deuterium is, the smaller the molecular hardcore volume is. Accordingly, the electrical polarizability may be reduced and the intermolecular interaction can be weakened, so that when a device is manufactured, the device has a stabler stacking structure.

These characteristics induce an effect of lowering the crystallinity by creating the amorphous state of a thin film. That is, the heterocyclic compound represented by Chemical Formula 1 may be effective in improving the heat resistance of an OLED device, thereby improving the service life and driving characteristics.

In an exemplary embodiment of the present application, provided is a heterocyclic compound represented by the following Chemical Formula 1.

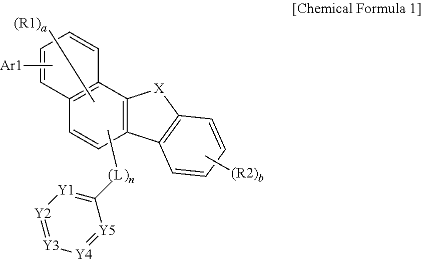

In Chemical Formula 1,
X is O, S or —C(Ra)(Rb), and Ra and Rb are the same as or different from each other, and are each independently a substituted or unsubstituted C1 to C60 alkyl group or a substituted or unsubstituted C6 to C60 aryl group,
L is a direct bond; or a substituted or unsubstituted C6 to C60 arylene group,
n is an integer from 1 to 3, and when n is 2 or higher, substituents in the parenthesis are the same as or different from each other,
Y1 is CR11 or N, Y2 is CR12 or N, Y3 is CR13 or N, Y4 is CR14 or N, Y5 is CR15 or N, and at least one of Y1 to Y5 is N,
R11 to R15 are the same as or different from each other, and are each independently hydrogen; deuterium; a substituted or unsubstituted C6 to C60 aryl group; or a substituted or unsubstituted C2 to C60 heteroaryl group, or two or more adjacent groups are bonded to each other to form a substituted or unsubstituted C2 to C60 hetero ring,
Ar1 is a substituted or unsubstituted C6 to C60 aryl group; or a substituted or unsubstituted C2 to C60 heteroaryl group,
R1 and R2 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or two or more adjacent groups are bonded to each other to form a substituted or unsubstituted aliphatic or aromatic hydrocarbon ring or hetero ring,
a is an integer from 0 to 4, and when a is 2 or higher, substituents in the parenthesis are the same as or different from each other, and
b is an integer from 0 to 4, and when b is 2 or higher, substituents in the parenthesis are the same as or different from each other.

In an exemplary embodiment of the present application, Chemical Formula 1 may be represented by any one of the following Chemical Formulae 1-1 and 1-2.

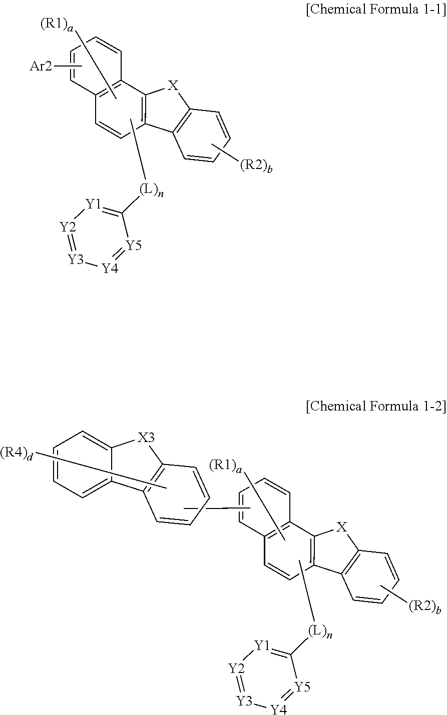

In Chemical Formulae 1-1 and 1-2,
the definitions of X, R1 and R2, L, Y1 to Y5, a, b, and n are the same as the definitions in Chemical Formula 1,
X3 is O or S,
Ar2 is a substituted or unsubstituted C6 to C60 aryl group,
R4 is each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or two or more adjacent groups are bonded to each other to form a substituted or unsubstituted aliphatic or aromatic hydrocarbon ring or hetero ring, and
d is an integer from 0 to 7, and when d is 2 or higher, substituents in the parenthesis are the same as or different from each other.

In an exemplary embodiment of the present application, X is O, S or —C(Ra)(Rb), and Ra and Rb are the same as or different from each other, and may be each independently a substituted or unsubstituted C1 to C40 alkyl group or a substituted or unsubstituted C6 to C40 aryl group.

In another exemplary embodiment, X is O, S or —C(Ra)(Rb), and Ra and Rb are the same as or different from each other, and may be each independently a substituted or unsubstituted C1 to C20 alkyl group or a substituted or unsubstituted C6 to C20 aryl group.

In still another exemplary embodiment, X is O, S or —C(Ra)(Rb), and Ra and Rb are the same as or different from each other, and may be each independently a substituted or unsubstituted methyl group; a substituted or unsubstituted ethyl group; a substituted or unsubstituted propyl group; or a substituted or unsubstituted phenyl group.

In yet another exemplary embodiment, X is O, S or —C(Ra)(Rb), and Ra and Rb are the same as or different from each other, and may be each independently a methyl group; an ethyl group; or a propyl group.

In still yet another exemplary embodiment, X is O, S or —C(Ra)(Rb), and Ra and Rb may be a methyl group.

In a further exemplary embodiment, X may be O or S.

In another further exemplary embodiment, X is —C(Ra)(Rb), and Ra and Rb may be a methyl group.

In an exemplary embodiment of the present application, L may be a direct bond; or a substituted or unsubstituted C6 to C40 arylene group.

In another exemplary embodiment, L may be a direct bond; or a substituted or unsubstituted C6 to C20 arylene group.

In still another exemplary embodiment, L may be a direct bond; a substituted or unsubstituted phenylene group; a substituted or unsubstituted naphthylene group; or a substituted or unsubstituted biphenylene group.

In yet another exemplary embodiment, L may be a direct bond; a phenylene group; a naphthylene group; or a biphenylene group.

In an exemplary embodiment of the present application, n is an integer from 1 to 2, and when n is 2 or higher, substituents in the parenthesis may be the same as or different from each other.

In another exemplary embodiment, n is 2, and substituents in the parenthesis may be the same as or different from each other.

In still another exemplary embodiment, n may be 1.

In an exemplary embodiment of the present application, Y1 is CR11 or N, Y2 is CR12 or N, Y3 is CR13 or N, Y4 is CR14 or N, Y5 is CR15 or N, and at least two of Y1 to Y5 may be N.

In another exemplary embodiment, Y1 is CR11 or N, Y2 is CR12 or N, Y3 is CR13 or N, Y4 is CR14 or N, Y5 is CR15 or N, and at least two of Y1, Y3 and Y5 may be N.

In still another exemplary embodiment, Y1 is C R11 or N, Y2 is C R12, Y3 is C R13 or N, Y4 is N, Y5 is CR15 or N, and at least two of Y1, Y3 and Y5 may be N.

In yet another exemplary embodiment, Y1 and Y5 are N, Y2 is C R12, Y3 is C R13, and Y4 may be CR14.

In still yet another exemplary embodiment, Y1 and Y3 are N, Y2 is CR12, Y4 is CR14, and Y5 may be C R15.

In a further exemplary embodiment, Y1 is C R11, Y2 is C R12, Y3 and Y5 are N, and Y4 may be CR14.

In another further exemplary embodiment, Y1, Y3 and Y5 are N, Y2 is C R12, and Y4 may be CR14.

In an exemplary embodiment of the present application, R11 to R15 are the same as or different from each other, and are each independently hydrogen; deuterium; a substituted or unsubstituted C6 to C40 aryl group; or a substituted or unsubstituted C2 to C40 heteroaryl group, or two or more adjacent groups may be bonded to each other to form a substituted or unsubstituted C2 to C40 hetero ring.

In another exemplary embodiment, R11 to R15 are the same as or different from each other, and are each independently hydrogen; deuterium; a substituted or unsubstituted C6 to C20 aryl group; or a substituted or unsubstituted C2 to C20 heteroaryl group, or two or more adjacent groups may be bonded to each other to form a substituted or unsubstituted C2 to C20 hetero ring.

In still another exemplary embodiment, R11 to R15 are the same as or different from each other, and are each independently hydrogen; deuterium; a substituted or unsubstituted phenyl group; a substituted or unsubstituted naphthyl group; a substituted or unsubstituted biphenyl group; a substituted or unsubstituted terphenyl group; a substituted or unsubstituted fluorenyl group; a substituted or unsubstituted triphenylenyl group; a substituted or unsubstituted phenanthrenyl group; a substituted or unsubstituted dibenzofuran group; a substituted or unsubstituted dibenzothiophene group; or a substituted or unsubstituted carbazole group, or two or more adjacent groups may be bonded to each other to form a substituted or unsubstituted C2 to C20 hetero ring.

In yet another exemplary embodiment, R11 to R15 are the same as or different from each other, and are each independently hydrogen; deuterium; a phenyl group which is unsubstituted or substituted with deuterium or a C6 to C20 aryl group; a naphthyl group which is unsubstituted or substituted with deuterium or a C6 to C20 aryl group; a biphenyl group which is unsubstituted or substituted with deuterium; a terphenyl group which is unsubstituted or substituted with deuterium; a fluorenyl group which is unsubstituted or substituted with a C1 to C20 alkyl group; a triphenylenyl group which is unsubstituted or substituted with deuterium; a phenanthrenyl group which is unsubstituted or substituted with deuterium; a substituted or unsubstituted dibenzofuran group; a substituted or unsubstituted dibenzothiophene group; or a substituted or unsubstituted carbazole group, or two or more adjacent groups may be bonded to each other to form a two-membered hetero ring which has one or two heteroatom(s) selected from O and S and is substituted or unsubstituted.

In still yet another exemplary embodiment, R11 to R15 are the same as or different from each other, and are each independently hydrogen; deuterium; a phenyl group which is unsubstituted or substituted with deuterium or a naphthyl group unsubstituted or substituted with deuterium; a naphthyl group which is unsubstituted or substituted with deuterium or a phenyl group unsubstituted or substituted with deuterium; a biphenyl group which is unsubstituted or substituted with deuterium; a terphenyl group; a fluorenyl group substituted with a methyl group; a triphenylenyl group; a phenanthrenyl group; a dibenzofuran group; a dibenzothiophene group; or a carbazole group, or two or more adjacent groups may be bonded to each other to form a two-membered hetero ring which has one or two heteroatom(s) selected from O and S and is substituted or unsubstituted.

In an exemplary embodiment of the present application, Ar1 may be a substituted or unsubstituted C6 to C40 aryl group; or a substituted or unsubstituted C2 to C40 heteroaryl group.

In another exemplary embodiment, Ar1 may be a substituted or unsubstituted C6 to C20 aryl group; or a substituted or unsubstituted C2 to C20 heteroaryl group.

In still another exemplary embodiment, Ar1 may be a C6 to C20 aryl group which is unsubstituted or substituted with deuterium or a C6 to C20 aryl group; or a substituted or unsubstituted C2 to C20 heteroaryl group.

In yet another exemplary embodiment, Ar1 may be a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

In still yet another exemplary embodiment, Ar1 may be a phenyl group which is unsubstituted or substituted with deuterium or a naphthyl group; a biphenyl group; a terphenyl group; a naphthyl group which is unsubstituted or substituted with deuterium or a phenyl group; a dibenzofuran group; or a dibenzothiophene group.

In an exemplary embodiment of the present application, Ar2 may be a substituted or unsubstituted C6 to C40 aryl group.

In another exemplary embodiment, Ar2 may be a substituted or unsubstituted C6 to C20 aryl group.

In still another exemplary embodiment, Ar2 may be a C6 to C20 aryl group which is unsubstituted or substituted with deuterium or a C6 to C20 aryl group.

In yet another exemplary embodiment, Ar2 may be a substituted or unsubstituted phenyl group; a substituted or unsubstituted biphenyl group; a substituted or unsubstituted terphenyl group; or a substituted or unsubstituted naphthyl group.

In still yet another exemplary embodiment, Ar2 may be a phenyl group which is unsubstituted or substituted with deuterium or a naphthyl group; a biphenyl group; a terphenyl group; or a naphthyl group which is unsubstituted or substituted with deuterium or a phenyl group.

In an exemplary embodiment of the present application, R1 and R2 are the same as or different from each other, and are each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group, or two or more adjacent groups may be bonded to each other to form a substituted or unsubstituted aliphatic or aromatic hydrocarbon ring or hetero ring.

In another exemplary embodiment, R1 and R2 are the same as or different from each other, and may be each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, R1 and R2 are the same as or different from each other, and may be each independently hydrogen; deuterium; a halogen group; a cyano group; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another exemplary embodiment, R1 and R2 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In still yet another exemplary embodiment, R1 and R2 are the same as or different from each other, and may be each independently hydrogen; deuterium; a substituted or unsubstituted methyl group; a substituted or unsubstituted ethyl group; a substituted or unsubstituted propyl group; a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted triphenylene group; a substituted or unsubstituted dibenzofuran group; a substituted or unsubstituted dibenzothiophene group; or a substituted or unsubstituted carbazole group.

In a further exemplary embodiment, R1 and R2 are the same as or different from each other, and may be each independently hydrogen; deuterium; a methyl group; an ethyl group; a propyl group; a phenyl group; a biphenyl group; a terphenyl group; a naphthyl group; a phenanthrene group; a triphenylene group; a dibenzofuran group; a dibenzothiophene group; or a carbazole group.

In another further exemplary embodiment, R1 and R2 may be hydrogen; or deuterium.

In still another further exemplary embodiment, R1 and R2 may be hydrogen.

In yet another further exemplary embodiment, R1 and R2 may be deuterium.

In an exemplary embodiment of the present application, a is an integer from 0 to 3, and when a is 2 or higher, substituents in the parenthesis may be the same as or different from each other.

In an exemplary embodiment of the present application, a is an integer from 0 to 2, and when a is 2 or higher, substituents in the parenthesis may be the same as or different from each other.

In an exemplary embodiment of the present application, a is 1.

In an exemplary embodiment of the present application, a is 0.

In an exemplary embodiment of the present application, b is an integer from 0 to 3, and when b is 2 or higher, substituents in the parenthesis may be the same as or different from each other.

In an exemplary embodiment of the present application, b is an integer from 0 to 2, and when b is 2 or higher, substituents in the parenthesis may be the same as or different from each other.

In an exemplary embodiment of the present application, b is 1.

In an exemplary embodiment of the present application, b is 0.

In an exemplary embodiment of the present application, R4 is each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or two or more adjacent groups may be bonded to each other to form a substituted or unsubstituted aliphatic or aromatic hydrocarbon ring or hetero ring.

In an exemplary embodiment of the present application, R4 may be each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, R4 may be each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In still another exemplary embodiment, R4 may be each independently hydrogen; or deuterium.

In an exemplary embodiment of the present application, X3 may be 0 or S.

In an exemplary embodiment of the present application, the deuterium content of the heterocyclic compound of Chemical Formula 1 may be 0%, or 10% to 100%.

In an exemplary embodiment of the present application, the deuterium content of the heterocyclic compound of Chemical Formula 1 may be 0%, or 30% to 100%.

In an exemplary embodiment of the present application, the deuterium content of the heterocyclic compound of Chemical Formula 1 may be 0%, or 50% to 100%.

In an exemplary embodiment of the present application, the deuterium content of the heterocyclic compound of Chemical Formula 1 may be 0%, or 70% to 100%.

In an exemplary embodiment of the present application, the deuterium content of the heterocyclic compound of Chemical Formula 1 may be 0%, or 90% to 100%.

In an exemplary embodiment of the present application, the deuterium content of the heterocyclic compound of Chemical Formula 1 may be 0%.

In an exemplary embodiment of the present application, the deuterium content of the heterocyclic compound of Chemical Formula 1 may be 10% to 100%.

In an exemplary embodiment of the present application, the deuterium content of the heterocyclic compound of Chemical Formula 1 may be 30% to 100%.

In an exemplary embodiment of the present application, the deuterium content of the heterocyclic compound of Chemical Formula 1 may be 70% to 100%.

In an exemplary embodiment of the present application, the deuterium content of the heterocyclic compound of Chemical Formula 1 may be 90% to 100%.

In an exemplary embodiment of the present application, Chemical Formula 1 may be represented by any one of the following Chemical Formulae 1-3 to 1-6.

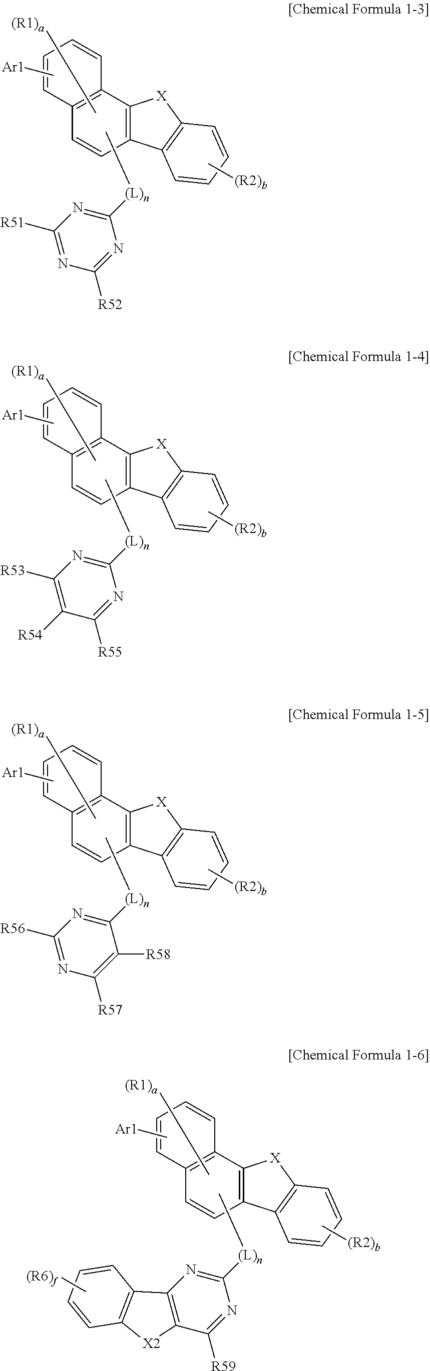

In Chemical Formulae 1-3 to 1-6,
the definitions of X, Ar1, R1, R2, L, a, b, and n are the same as the definitions in Chemical Formula 1,
X2 is O or S,
R51 to R59 are the same as or different from each other, and are each independently hydrogen; deuterium; a substituted or unsubstituted C6 to C60 aryl group; or a substituted or unsubstituted C2 to C60 heteroaryl group,
R6 is hydrogen; deuterium; a substituted or unsubstituted C6 to C60 aryl group; or a substituted or unsubstituted C2 to C60 heteroaryl group, and
f is an integer from 0 to 4, and when f is 2 or higher, substituents in the parenthesis are the same as or different from each other.

In an exemplary embodiment of the present application, R51 to R59 are the same as or different from each other, and may be each independently hydrogen; deuterium; a substituted or unsubstituted C6 to C40 aryl group; or a substituted or unsubstituted C2 to C40 heteroaryl group.

In another exemplary embodiment, R51 to R59 are the same as or different from each other, and may be each independently hydrogen; deuterium; a substituted or unsubstituted C6 to C20 aryl group; or a substituted or unsubstituted C2 to C20 heteroaryl group.

In still another exemplary embodiment, R51 to R59 are the same as or different from each other, and may be each independently hydrogen; deuterium; a substituted or unsubstituted phenyl group; a substituted or unsubstituted naphthyl group; a substituted or unsubstituted biphenyl group; a substituted or unsubstituted terphenyl group; a substituted or unsubstituted fluorenyl group; a substituted or unsubstituted triphenylenyl group; a substituted or unsubstituted phenanthrenyl group; a substituted or unsubstituted dibenzofuran group; a substituted or unsubstituted dibenzothiophene group; or a substituted or unsubstituted carbazole group.

In yet another exemplary embodiment, R51 to R59 are the same as or different from each other, and may be each independently hydrogen; deuterium; a phenyl group which is unsubstituted or substituted with deuterium or a C6 to C20 aryl group; a naphthyl group which is unsubstituted or substituted with deuterium or a C6 to C20 aryl group; a biphenyl group which is unsubstituted or substituted with deuterium; a terphenyl group which is unsubstituted or substituted with deuterium; a fluorenyl group which is unsubstituted or substituted with a C1 to C20 alkyl group; a triphenylenyl group which is unsubstituted or substituted with deuterium; a phenanthrenyl group which is unsubstituted or substituted with deuterium; a substituted or unsubstituted dibenzofuran group; a substituted or unsubstituted dibenzothiophene group; or a substituted or unsubstituted carbazole group.

In still yet another exemplary embodiment, R51 to R59 are the same as or different from each other, and may be each independently hydrogen; deuterium; a phenyl group which is unsubstituted or substituted with deuterium or a naphthyl group unsubstituted or substituted with deuterium; a naphthyl group which is unsubstituted or substituted with deuterium or a phenyl group unsubstituted or substituted with deuterium; a biphenyl group which is unsubstituted or substituted with deuterium; a terphenyl group; a fluorenyl group substituted with a methyl group; a triphenylenyl group; a phenanthrenyl group; a dibenzofuran group; a dibenzothiophene group; or a carbazole group.

In an exemplary embodiment of the present application, R6 may be hydrogen; deuterium; a substituted or unsubstituted C6 to C60 aryl group; or a substituted or unsubstituted C2 to C60 heteroaryl group.

In another exemplary embodiment, R6 may be hydrogen; deuterium; a substituted or unsubstituted C6 to C40 aryl group; or a substituted or unsubstituted C2 to C40 heteroaryl group.

In still another exemplary embodiment, R6 may be hydrogen; deuterium; a substituted or unsubstituted C6 to C20 aryl group; or a substituted or unsubstituted C2 to C20 heteroaryl group.

In yet another exemplary embodiment, R6 may be hydrogen; or deuterium.

In still yet another exemplary embodiment, R6 is hydrogen.

In a further exemplary embodiment, R6 is deuterium.

In an exemplary embodiment of the present application, provided is a heterocyclic compound in which Chemical Formula 1 is represented by any one of the following compounds.

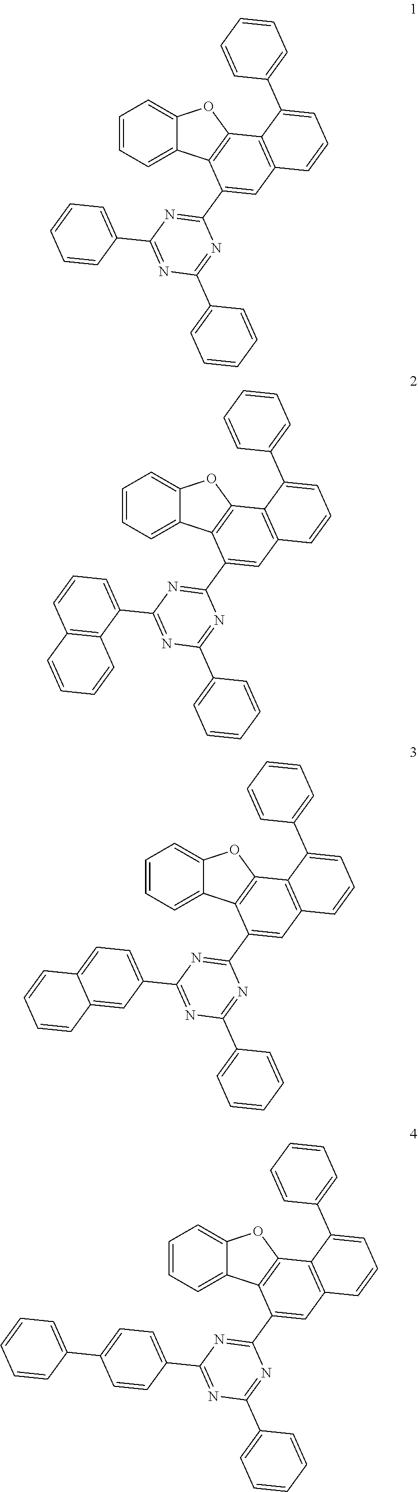

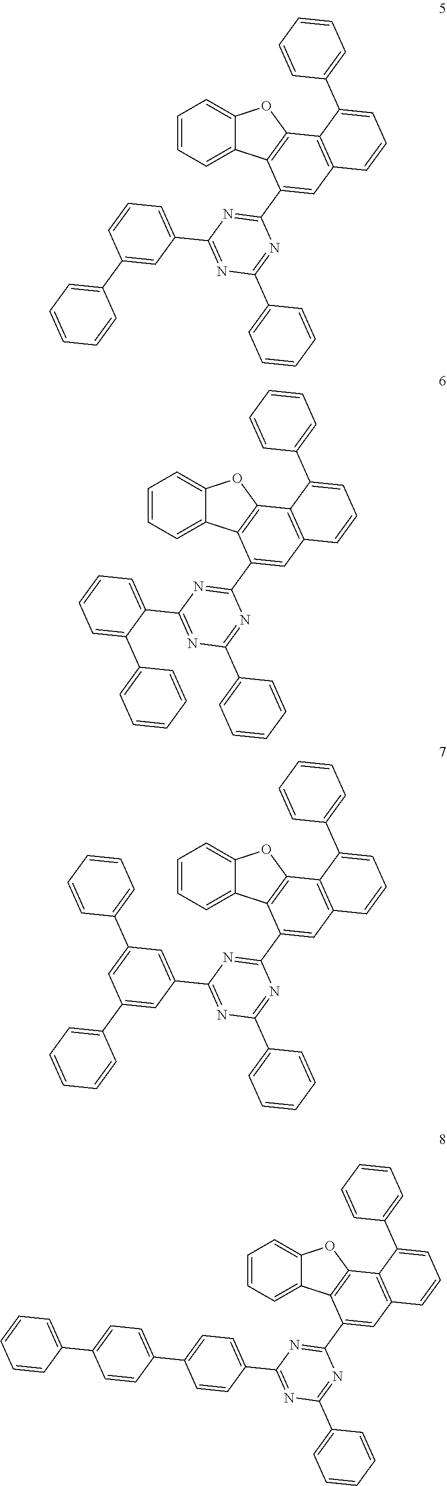

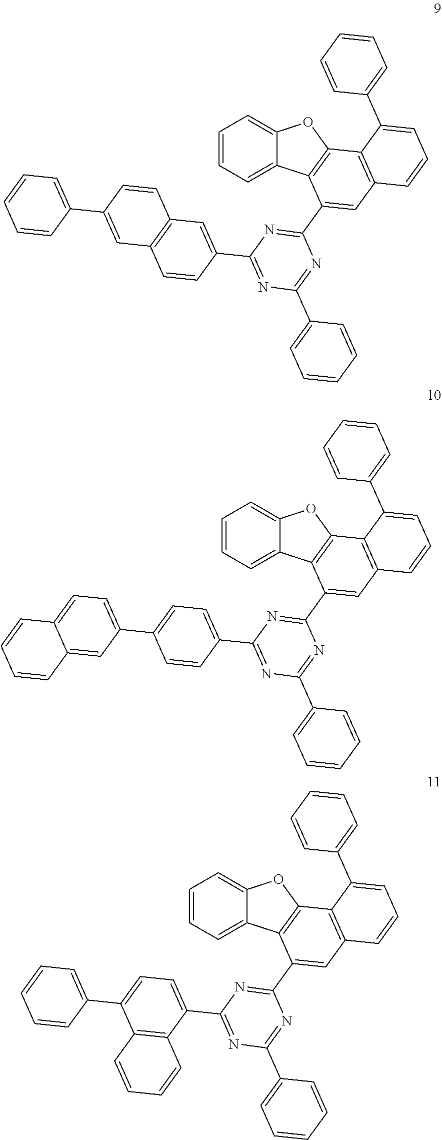

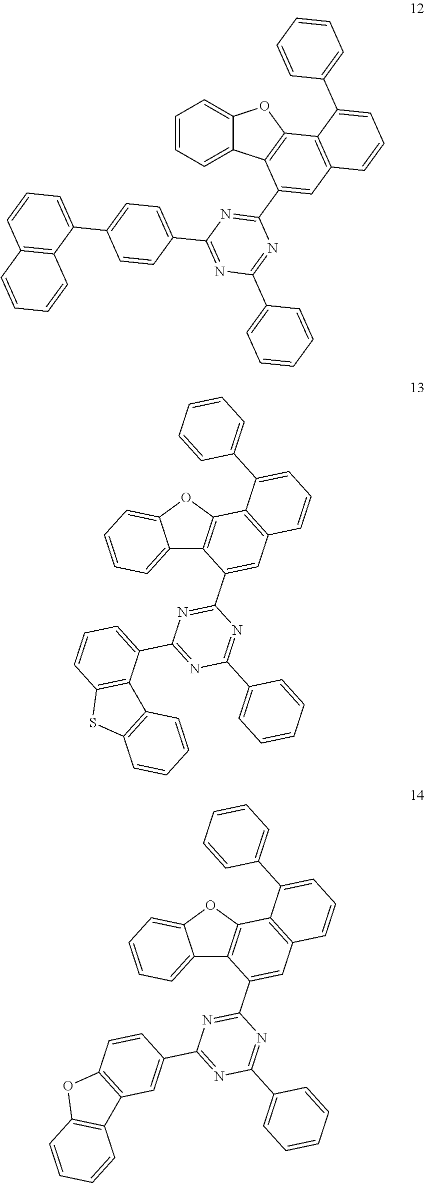 [SMILES: c1ccc(-c2nc(-c3ccc(-c4cccc5ccccc45)cc3)nc(-c3cc4cccc(-c5ccccc5)c4c4oc5ccccc5c34)n2)cc1 ; c1ccc(-c2nc(-c3cccc4sc5ccccc5c34)nc(-c3cc4cccc(-c5ccccc5)c4c4oc5ccccc5c34)n2)cc1 ; c1ccc(-c2nc(-c3ccc4oc5ccccc5c4c3)nc(-c3cc4cccc(-c5ccccc5)c4c4oc5ccccc5c34)n2)cc1]

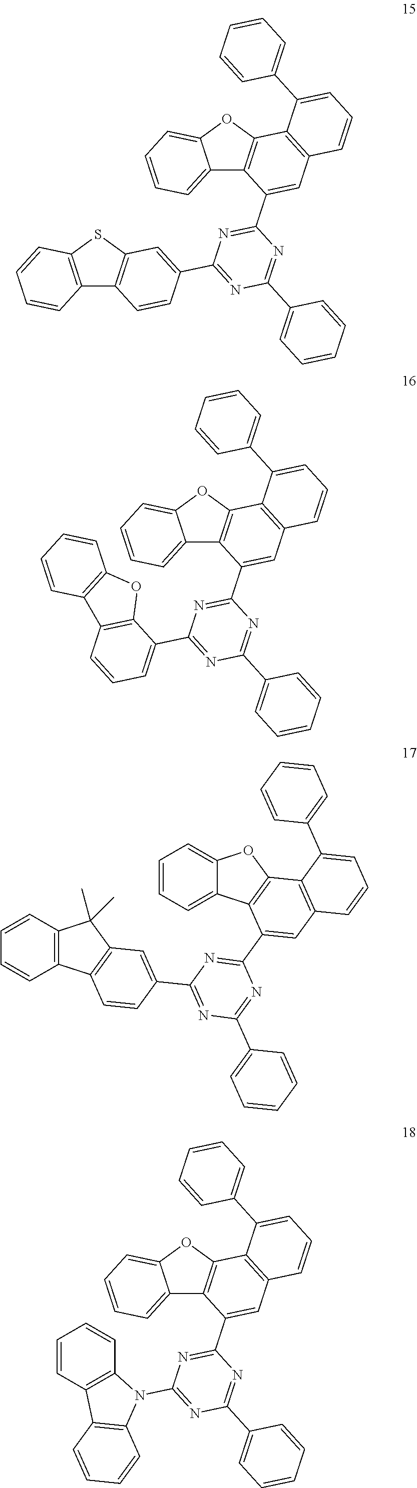

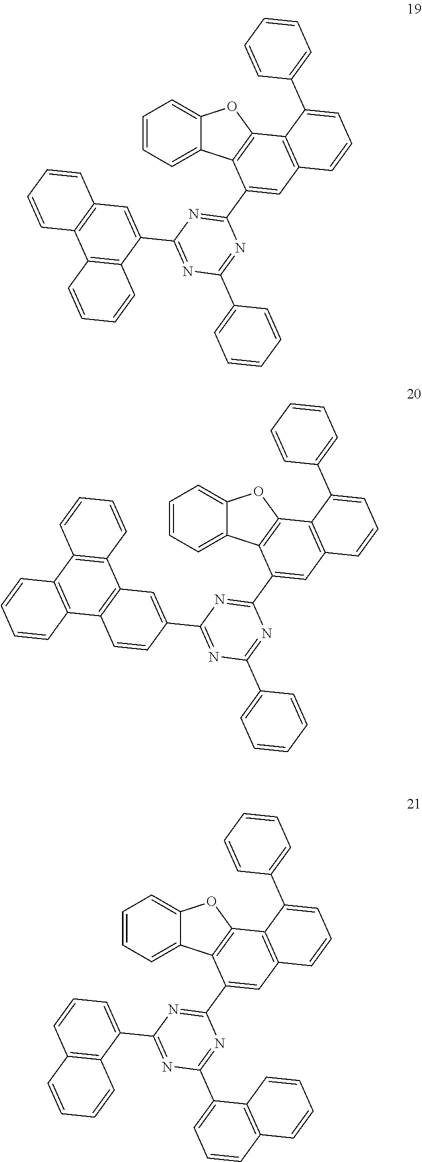 [SMILES: c1ccc(-c2nc(-c3cc4ccccc4c4ccccc34)nc(-c3cc4cccc(-c5ccccc5)c4c4oc5ccccc5c34)n2)cc1 ; c1ccc(-c2nc(-c3ccc4c5ccccc5c5ccccc5c4c3)nc(-c3cc4cccc(-c5ccccc5)c4c4oc5ccccc5c34)n2)cc1 ; c1ccc(-c2cccc3cc(-c4nc(-c5cccc6ccccc56)nc(-c5cccc6ccccc56)n4)c4c5ccccc5oc4c23)cc1]

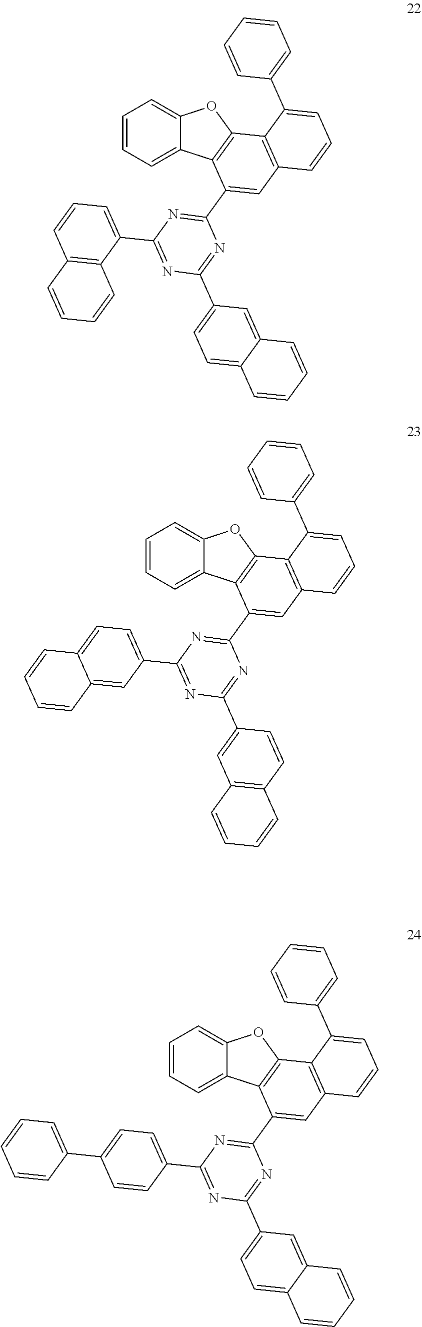

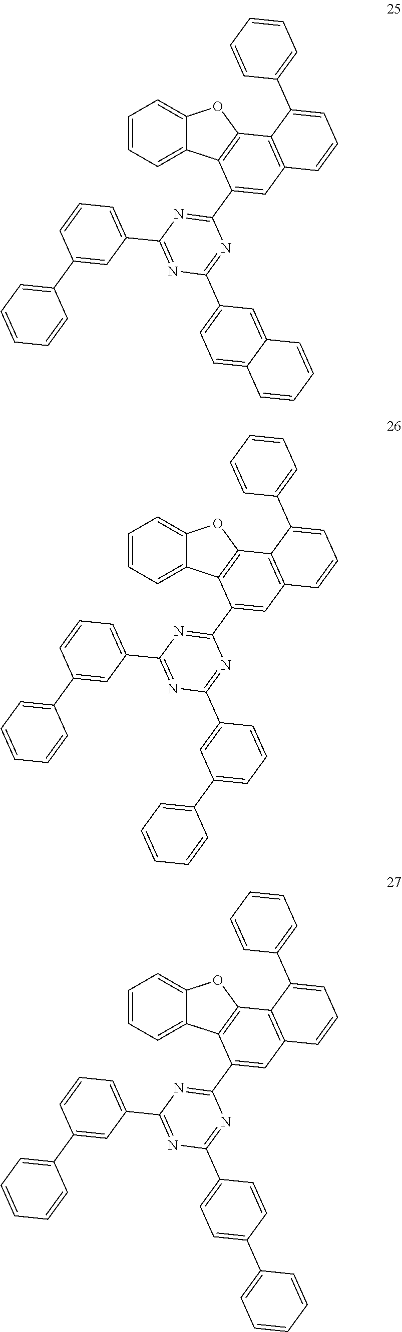

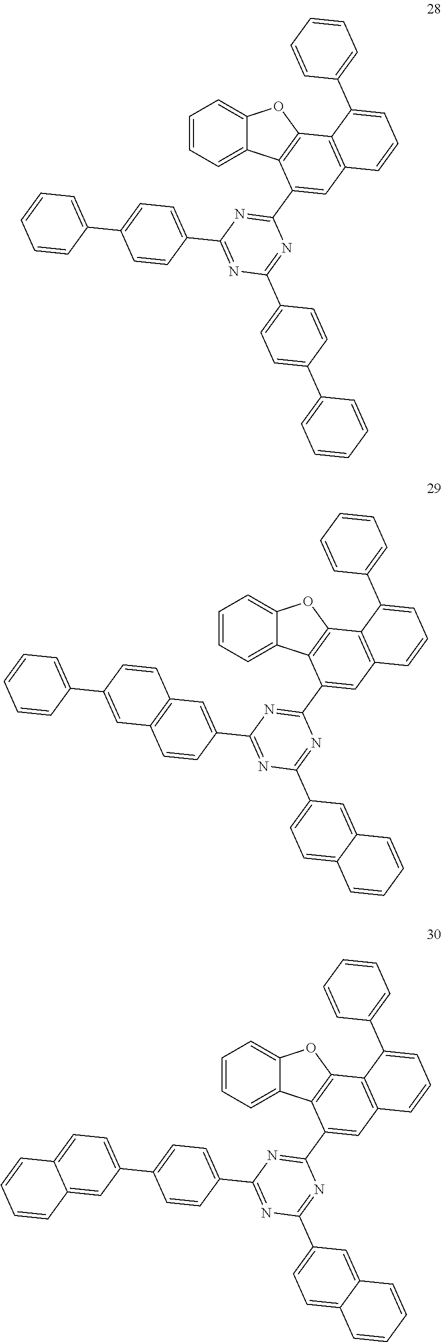 [SMILES: c1ccc(-c2ccc(-c3nc(-c4ccc(-c5ccccc5)cc4)nc(-c4cc5cccc(-c6ccccc6)c5c5oc6ccccc6c45)n3)cc2)cc1 ; c1ccc(-c2ccc3cc(-c4nc(-c5ccc6ccccc6c5)nc(-c5cc6cccc(-c7ccccc7)c6c6oc7ccccc7c56)n4)ccc3c2)cc1 ; c1ccc(-c2cccc3cc(-c4nc(-c5ccc(-c6ccc7ccccc7c6)cc5)nc(-c5ccc6ccccc6c5)n4)c4c5ccccc5oc4c23)cc1]

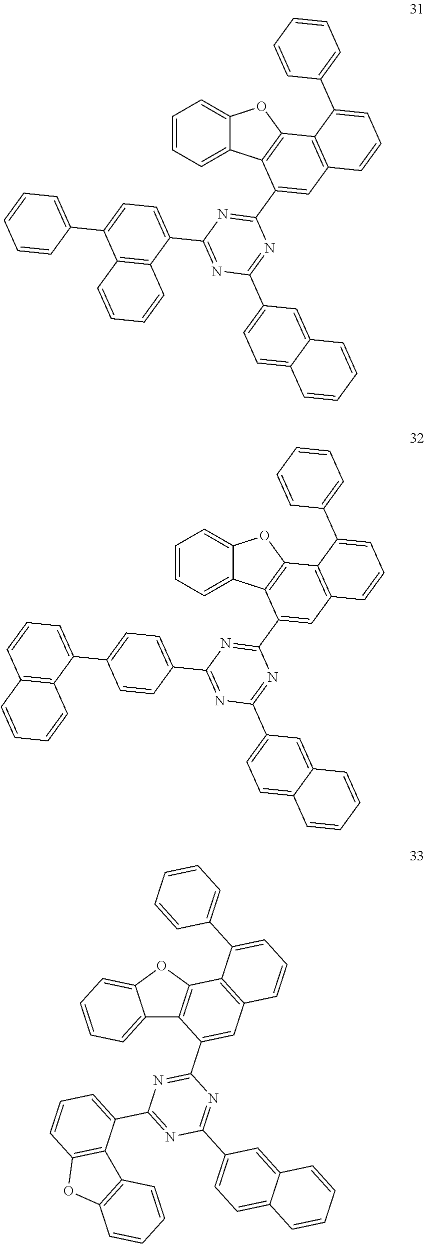 [SMILES: c1ccc(-c2ccc(-c3nc(-c4ccc5ccccc5c4)nc(-c4cc5cccc(-c6ccccc6)c5c5oc6ccccc6c45)n3)c3ccccc23)cc1 ; c1ccc(-c2cccc3cc(-c4nc(-c5ccc(-c6cccc7ccccc67)cc5)nc(-c5ccc6ccccc6c5)n4)c4c5ccccc5oc4c23)cc1 ; c1ccc(-c2cccc3cc(-c4nc(-c5ccc6ccccc6c5)nc(-c5cccc6oc7ccccc7c56)n4)c4c5ccccc5oc4c23)cc1]

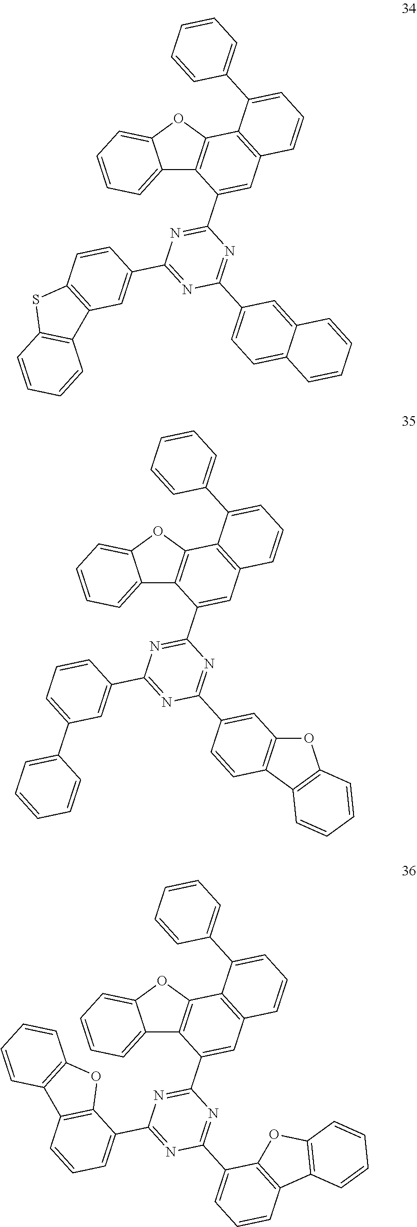 [SMILES: c1ccc(-c2cccc3cc(-c4nc(-c5ccc6ccccc6c5)nc(-c5ccc6sc7ccccc7c6c5)n4)c4c5ccccc5oc4c23)cc1 ; c1ccc(-c2cccc(-c3nc(-c4ccc5c(c4)oc4ccccc45)nc(-c4cc5cccc(-c6ccccc6)c5c5oc6ccccc6c45)n3)c2)cc1 ; c1ccc(-c2cccc3cc(-c4nc(-c5cccc6c5oc5ccccc56)nc(-c5cccc6c5oc5ccccc56)n4)c4c5ccccc5oc4c23)cc1]

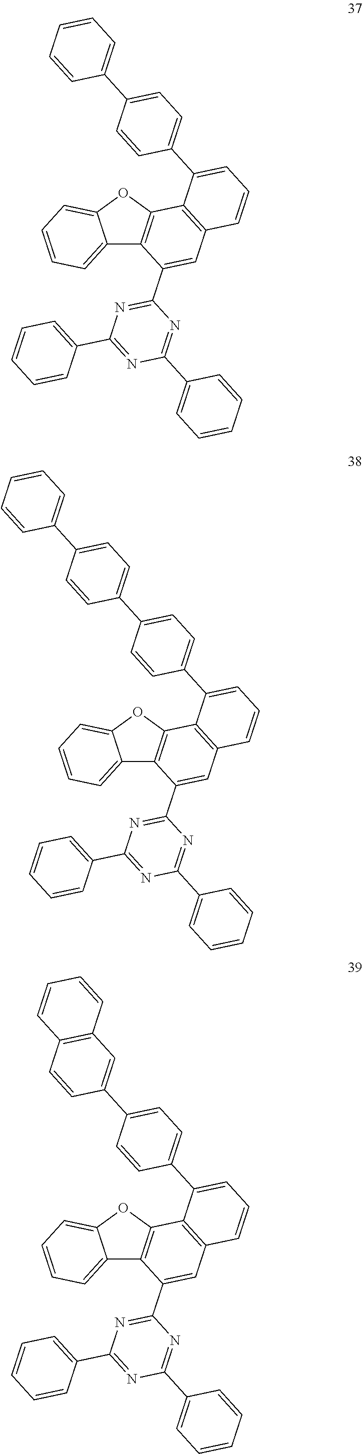 [SMILES: c1ccc(-c2ccc(-c3cccc4cc(-c5nc(-c6ccccc6)nc(-c6ccccc6)n5)c5c6ccccc6oc5c34)cc2)cc1 ; c1ccc(-c2ccc(-c3ccc(-c4cccc5cc(-c6nc(-c7ccccc7)nc(-c7ccccc7)n6)c6c7ccccc7oc6c45)cc3)cc2)cc1 ; c1ccc(-c2nc(-c3ccccc3)nc(-c3cc4cccc(-c5ccc(-c6ccc7ccccc7c6)cc5)c4c4oc5ccccc5c34)n2)cc1]

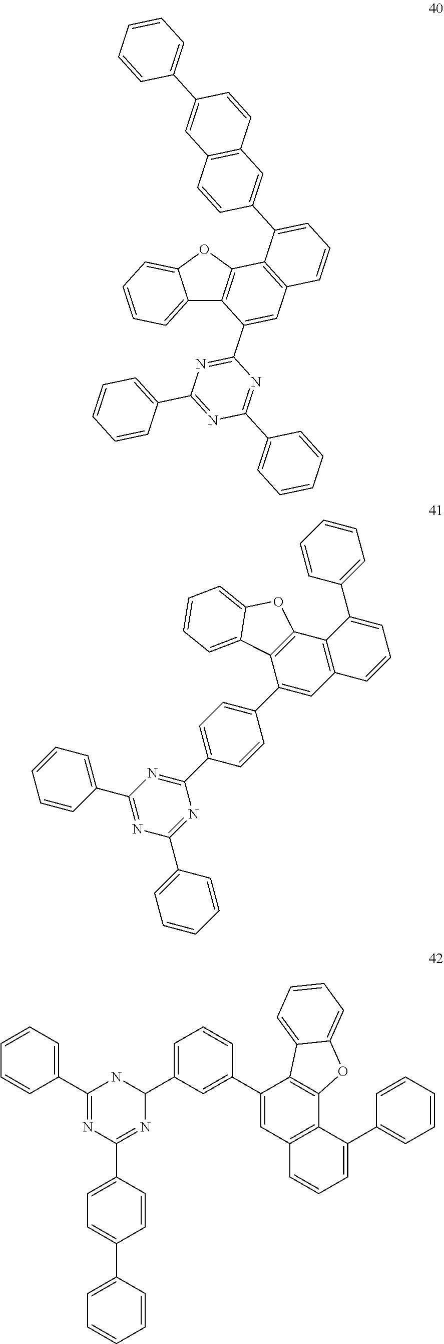

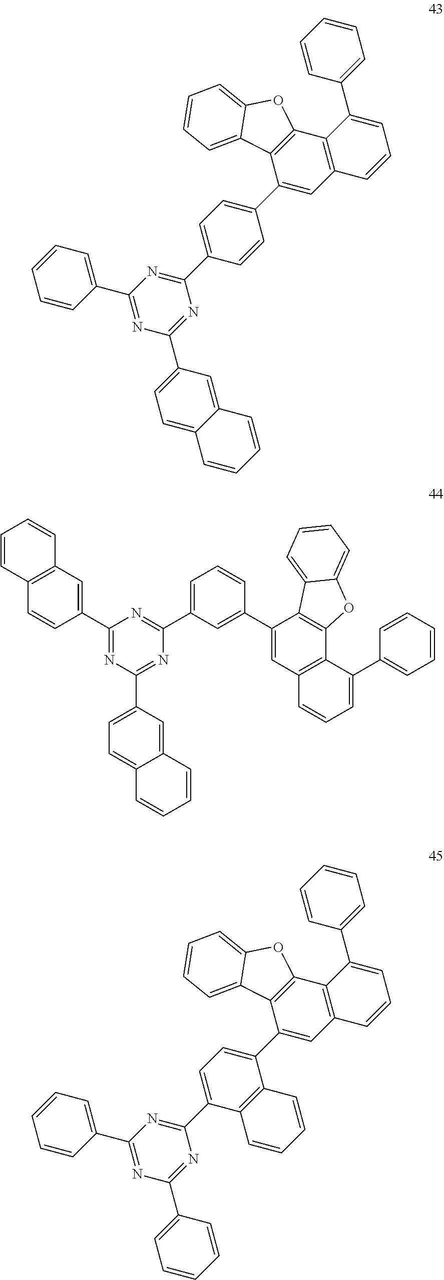 [SMILES: c1ccc(-c2nc(-c3ccc(-c4cc5cccc(-c6ccccc6)c5c5oc6ccccc6c45)cc3)nc(-c3ccc4ccccc4c3)n2)cc1 ; c1ccc(-c2cccc3cc(-c4cccc(-c5nc(-c6ccc7ccccc7c6)nc(-c6ccc7ccccc7c6)n5)c4)c4c5ccccc5oc4c23)cc1 ; c1ccc(-c2nc(-c3ccccc3)nc(-c3ccc(-c4cc5cccc(-c6ccccc6)c5c5oc6ccccc6c45)c4ccccc34)n2)cc1]

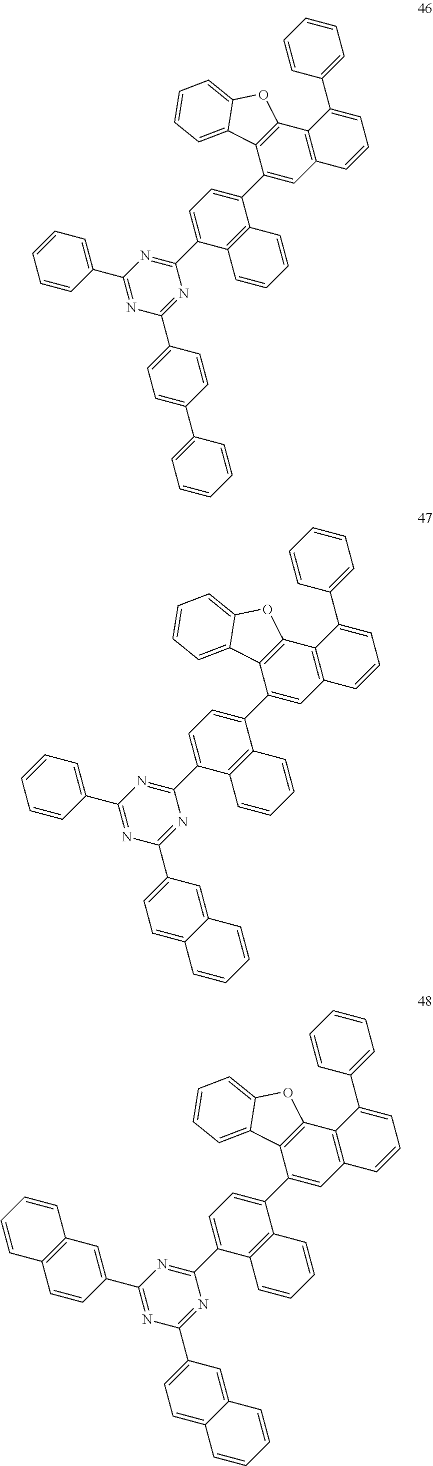 [SMILES: c1ccc(-c2ccc(-c3nc(-c4ccccc4)nc(-c4ccc(-c5cc6cccc(-c7ccccc7)c6c6oc7ccccc7c56)c5ccccc45)n3)cc2)cc1 ; c1ccc(-c2nc(-c3ccc4ccccc4c3)nc(-c3ccc(-c4cc5cccc(-c6ccccc6)c5c5oc6ccccc6c45)c4ccccc34)n2)cc1 ; c1ccc(-c2cccc3cc(-c4ccc(-c5nc(-c6ccc7ccccc7c6)nc(-c6ccc7ccccc7c6)n5)c5ccccc45)c4c5ccccc5oc4c23)cc1]

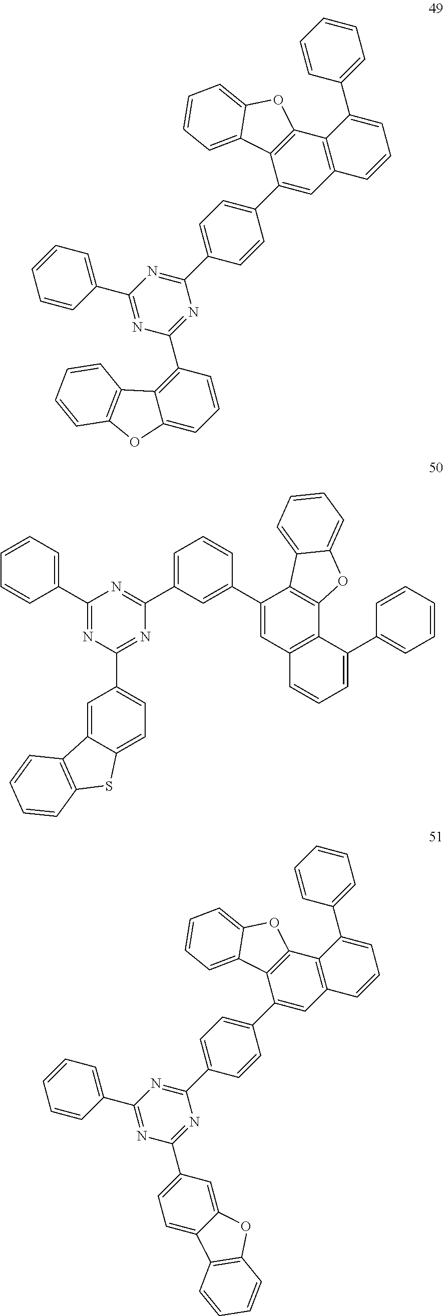 [SMILES: c1ccc(-c2nc(-c3ccc(-c4cc5cccc(-c6ccccc6)c5c5oc6ccccc6c45)cc3)nc(-c3cccc4oc5ccccc5c34)n2)cc1 ; c1ccc(-c2nc(-c3cccc(-c4cc5cccc(-c6ccccc6)c5c5oc6ccccc6c45)c3)nc(-c3ccc4sc5ccccc5c4c3)n2)cc1 ; c1ccc(-c2nc(-c3ccc(-c4cc5cccc(-c6ccccc6)c5c5oc6ccccc6c45)cc3)nc(-c3ccc4c(c3)oc3ccccc34)n2)cc1]

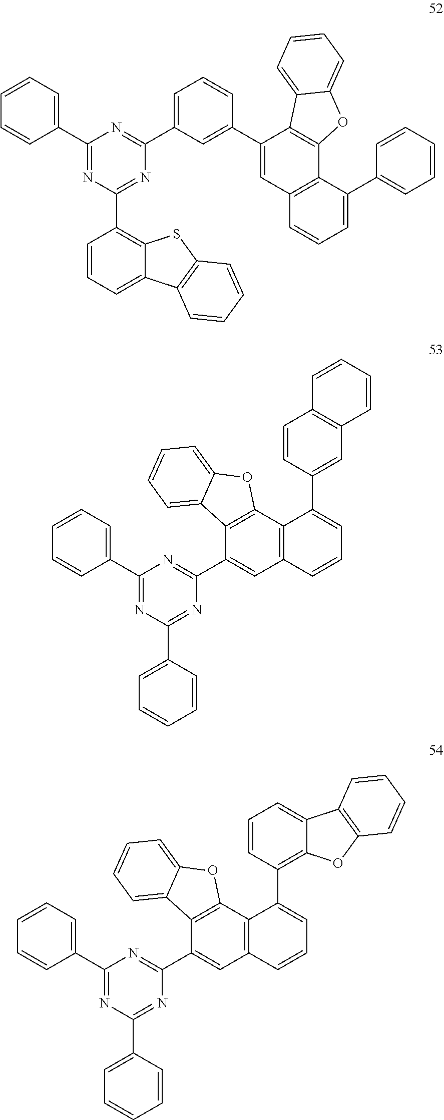 [SMILES: c1ccc(-c2nc(-c3cccc(-c4cc5cccc(-c6ccccc6)c5c5oc6ccccc6c45)c3)nc(-c3cccc4c3sc3ccccc34)n2)cc1 ; c1ccc(-c2nc(-c3ccccc3)nc(-c3cc4cccc(-c5ccc6ccccc6c5)c4c4oc5ccccc5c34)n2)cc1 ; c1ccc(-c2nc(-c3ccccc3)nc(-c3cc4cccc(-c5cccc6c5oc5ccccc56)c4c4oc5ccccc5c34)n2)cc1]

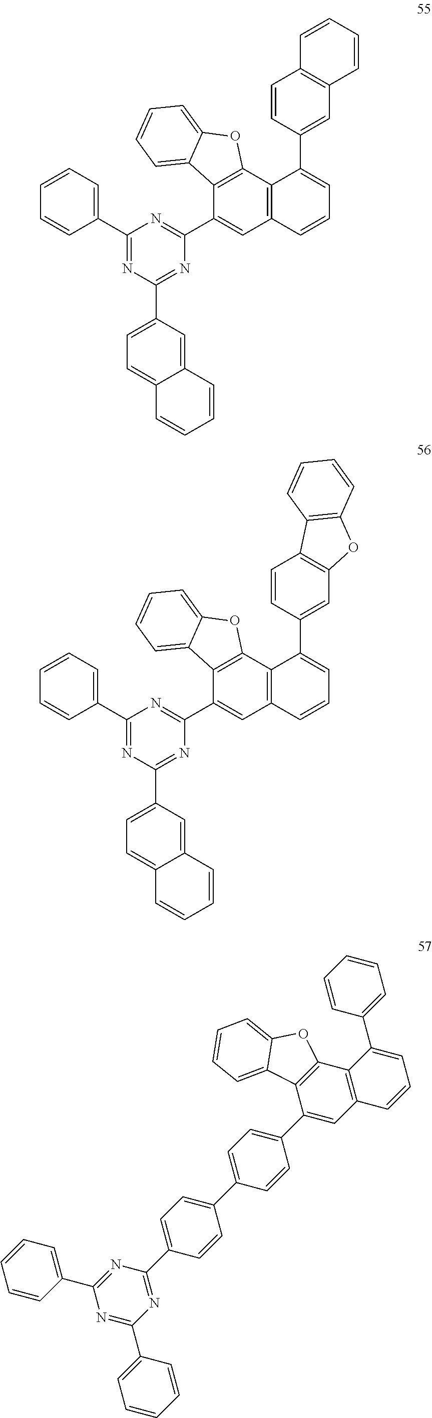 [SMILES: c1ccc(-c2nc(-c3ccc4ccccc4c3)nc(-c3cc4cccc(-c5ccc6ccccc6c5)c4c4oc5ccccc5c34)n2)cc1 ; c1ccc(-c2nc(-c3ccc4ccccc4c3)nc(-c3cc4cccc(-c5ccc6c(c5)oc5ccccc56)c4c4oc5ccccc5c34)n2)cc1 ; c1ccc(-c2nc(-c3ccccc3)nc(-c3ccc(-c4ccc(-c5cc6cccc(-c7ccccc7)c6c6oc7ccccc7c56)cc4)cc3)n2)cc1]

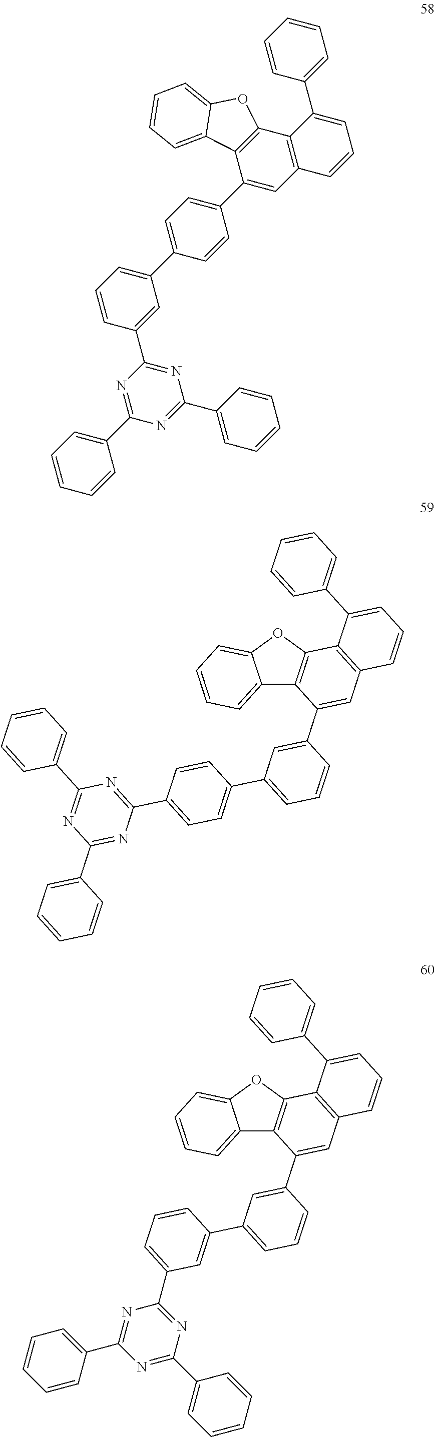 [SMILES: c1ccc(-c2nc(-c3ccccc3)nc(-c3cccc(-c4ccc(-c5cc6cccc(-c7ccccc7)c6c6oc7ccccc7c56)cc4)c3)n2)cc1 ; c1ccc(-c2nc(-c3ccccc3)nc(-c3ccc(-c4cccc(-c5cc6cccc(-c7ccccc7)c6c6oc7ccccc7c56)c4)cc3)n2)cc1 ; c1ccc(-c2nc(-c3ccccc3)nc(-c3cccc(-c4cccc(-c5cc6cccc(-c7ccccc7)c6c6oc7ccccc7c56)c4)c3)n2)cc1]

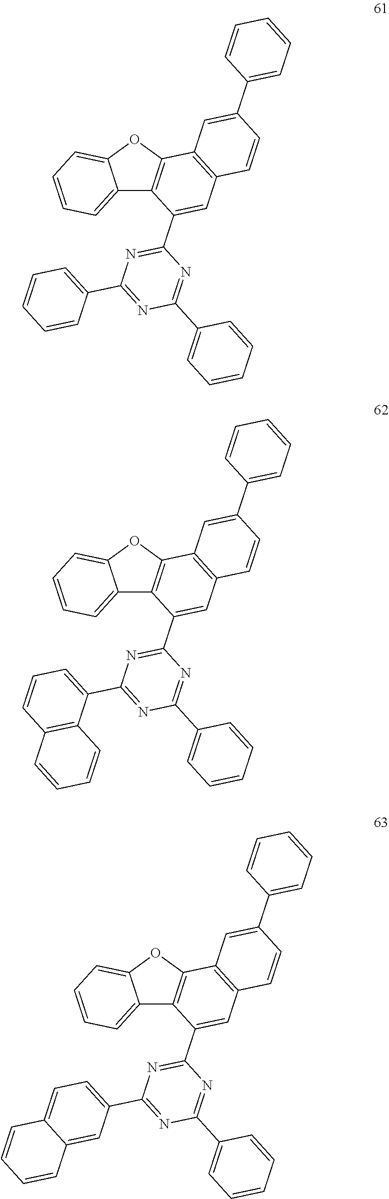 [SMILES: c1ccc(-c2ccc3cc(-c4nc(-c5ccccc5)nc(-c5ccccc5)n4)c4c5ccccc5oc4c3c2)cc1 ; c1ccc(-c2ccc3cc(-c4nc(-c5ccccc5)nc(-c5cccc6ccccc56)n4)c4c5ccccc5oc4c3c2)cc1 ; c1ccc(-c2ccc3cc(-c4nc(-c5ccccc5)nc(-c5ccc6ccccc6c5)n4)c4c5ccccc5oc4c3c2)cc1]

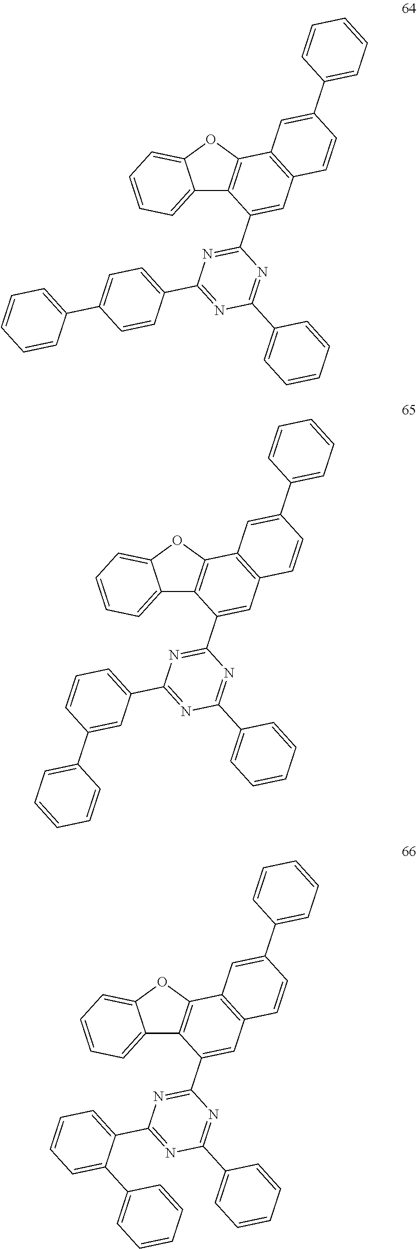

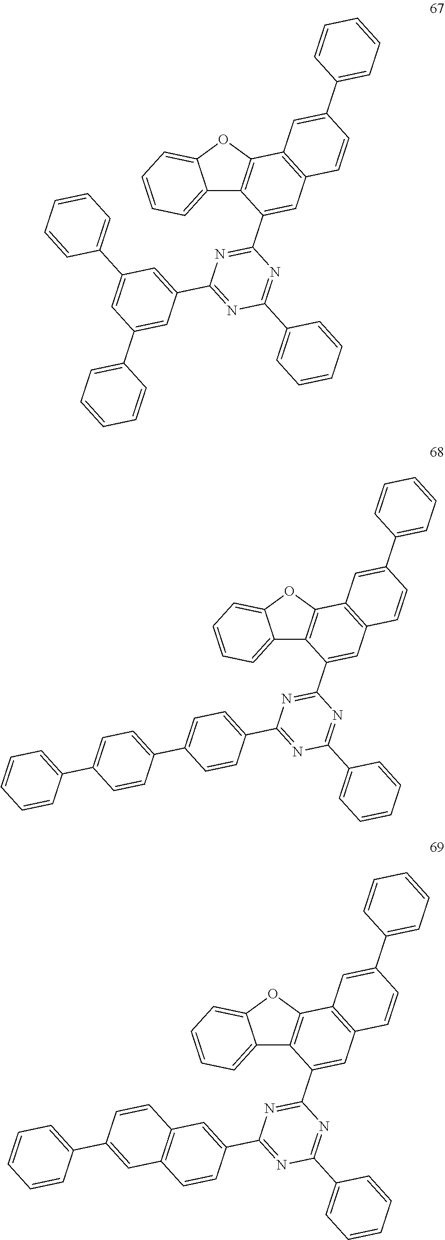 [SMILES: c1ccc(-c2cc(-c3ccccc3)cc(-c3nc(-c4ccccc4)nc(-c4cc5ccc(-c6ccccc6)cc5c5oc6ccccc6c45)n3)c2)cc1 ; c1ccc(-c2ccc(-c3ccc(-c4nc(-c5ccccc5)nc(-c5cc6ccc(-c7ccccc7)cc6c6oc7ccccc7c56)n4)cc3)cc2)cc1 ; c1ccc(-c2ccc3cc(-c4nc(-c5ccccc5)nc(-c5cc6ccc(-c7ccccc7)cc6c6oc7ccccc7c56)n4)ccc3c2)cc1]

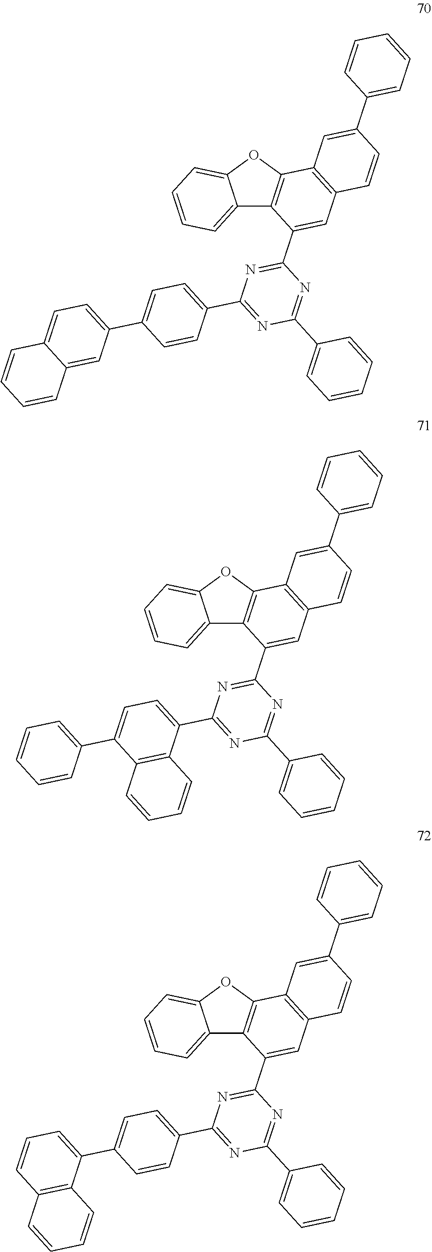 [SMILES: c1ccc(-c2ccc3cc(-c4nc(-c5ccccc5)nc(-c5ccc(-c6ccc7ccccc7c6)cc5)n4)c4c5ccccc5oc4c3c2)cc1 ; c1ccc(-c2ccc3cc(-c4nc(-c5ccccc5)nc(-c5ccc(-c6ccccc6)c6ccccc56)n4)c4c5ccccc5oc4c3c2)cc1 ; c1ccc(-c2ccc3cc(-c4nc(-c5ccccc5)nc(-c5ccc(-c6cccc7ccccc67)cc5)n4)c4c5ccccc5oc4c3c2)cc1]

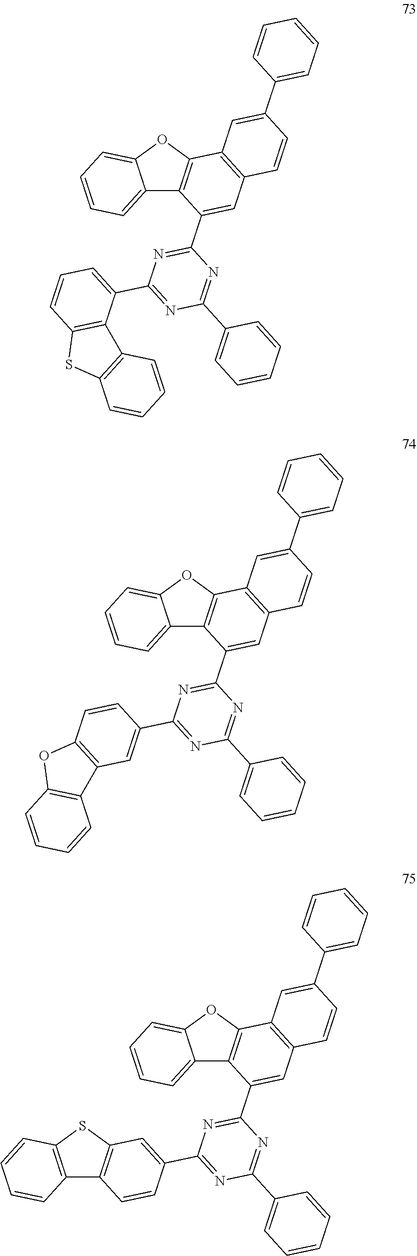 [SMILES: c1ccc(-c2ccc3cc(-c4nc(-c5ccccc5)nc(-c5cccc6sc7ccccc7c56)n4)c4c5ccccc5oc4c3c2)cc1 ; c1ccc(-c2ccc3cc(-c4nc(-c5ccccc5)nc(-c5ccc6oc7ccccc7c6c5)n4)c4c5ccccc5oc4c3c2)cc1 ; c1ccc(-c2ccc3cc(-c4nc(-c5ccccc5)nc(-c5ccc6c(c5)sc5ccccc56)n4)c4c5ccccc5oc4c3c2)cc1]

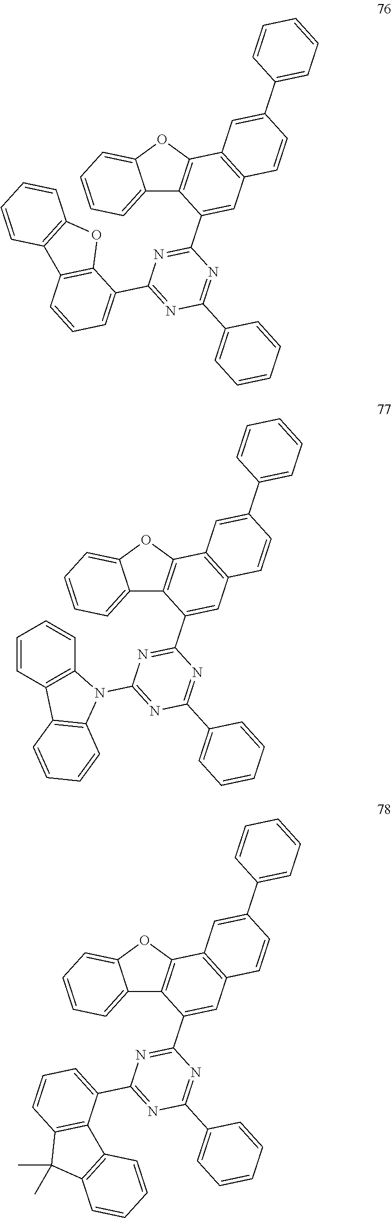

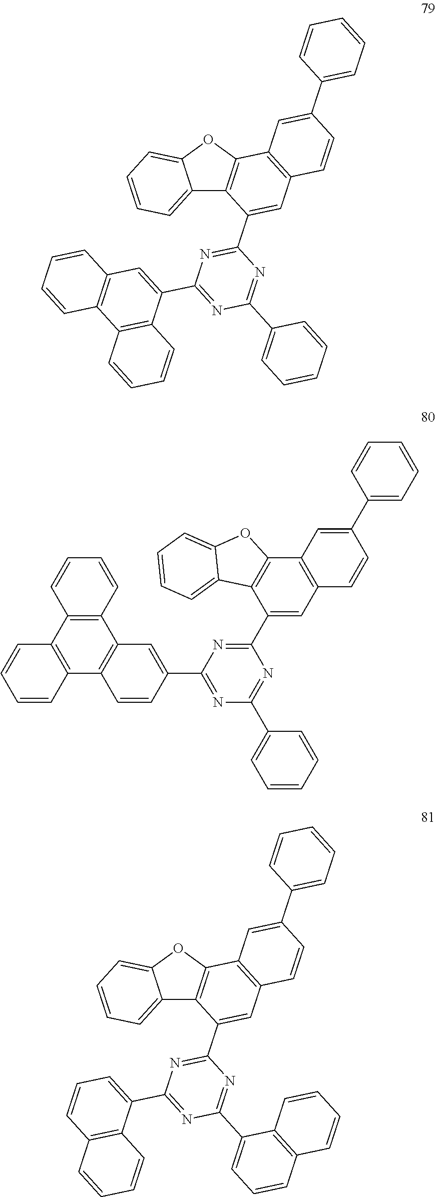

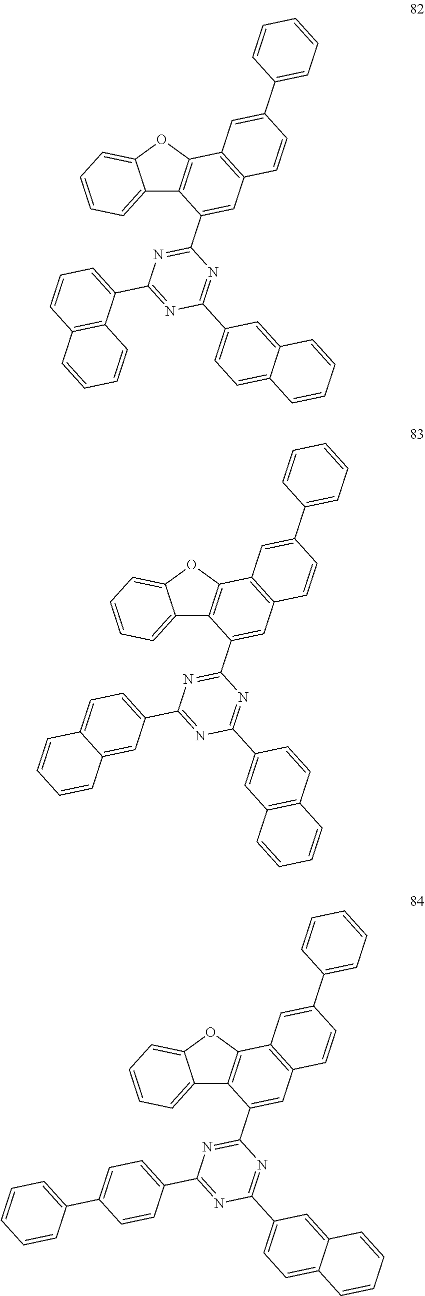 [SMILES: c1ccc(-c2ccc3cc(-c4nc(-c5ccc6ccccc6c5)nc(-c5cccc6ccccc56)n4)c4c5ccccc5oc4c3c2)cc1 ; c1ccc(-c2ccc3cc(-c4nc(-c5ccc6ccccc6c5)nc(-c5ccc6ccccc6c5)n4)c4c5ccccc5oc4c3c2)cc1 ; c1ccc(-c2ccc(-c3nc(-c4ccc5ccccc5c4)nc(-c4cc5ccc(-c6ccccc6)cc5c5oc6ccccc6c45)n3)cc2)cc1]

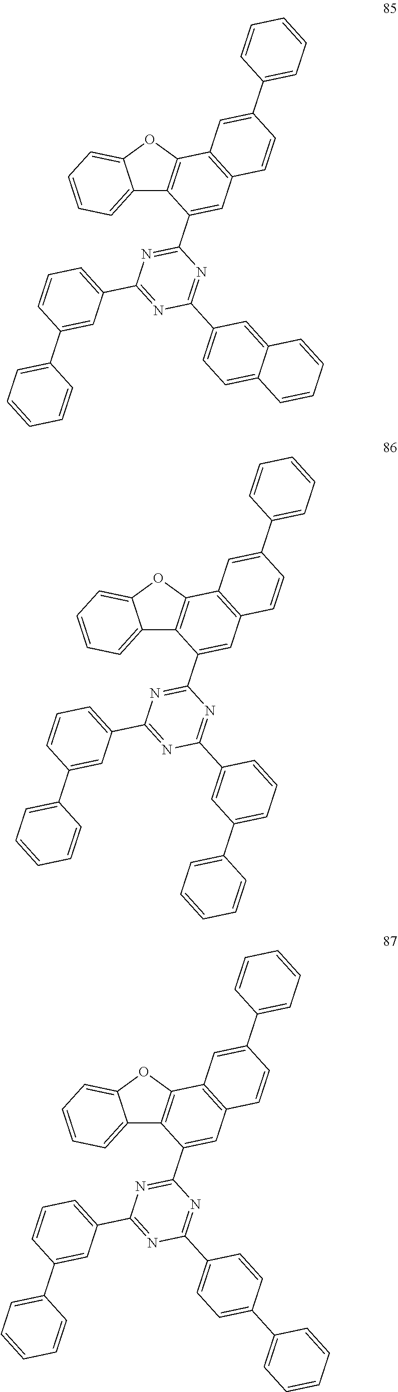

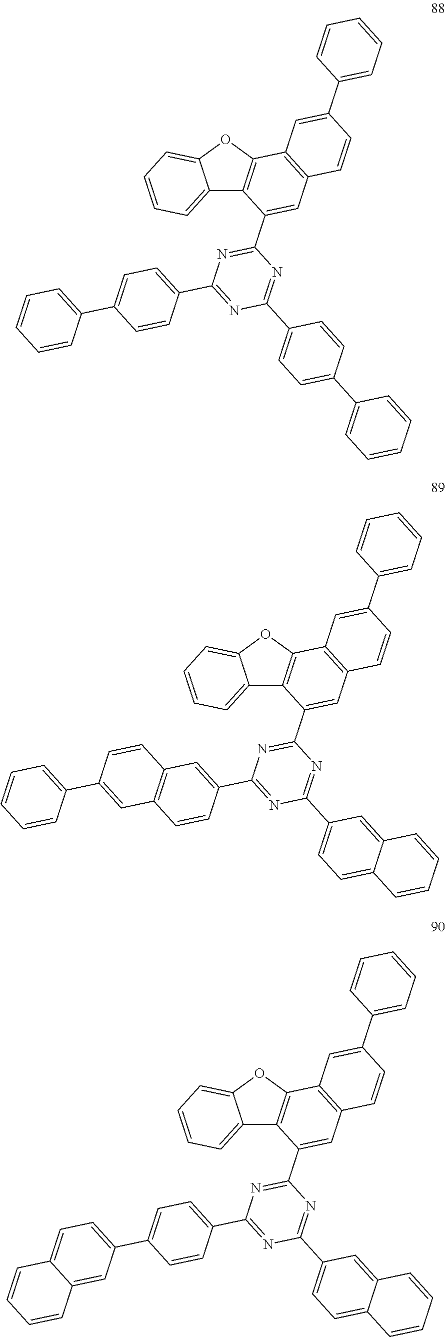 [SMILES: c1ccc(-c2ccc(-c3nc(-c4ccc(-c5ccccc5)cc4)nc(-c4cc5ccc(-c6ccccc6)cc5c5oc6ccccc6c45)n3)cc2)cc1 ; c1ccc(-c2ccc3cc(-c4nc(-c5ccc6ccccc6c5)nc(-c5cc6ccc(-c7ccccc7)cc6c6oc7ccccc7c56)n4)ccc3c2)cc1 ; c1ccc(-c2ccc3cc(-c4nc(-c5ccc(-c6ccc7ccccc7c6)cc5)nc(-c5ccc6ccccc6c5)n4)c4c5ccccc5oc4c3c2)cc1]

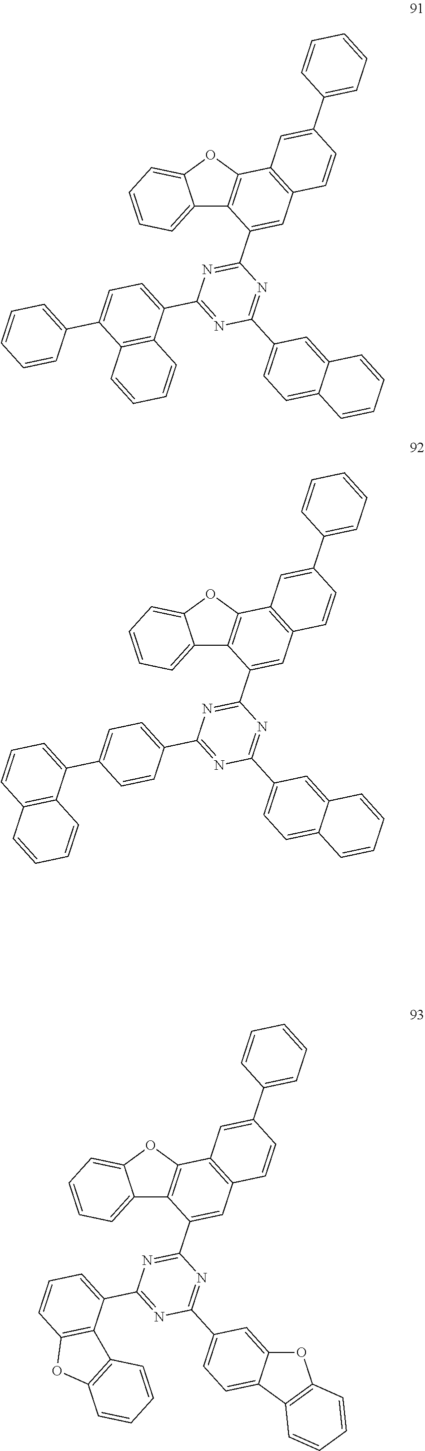 [SMILES: c1ccc(-c2ccc3cc(-c4nc(-c5ccc6ccccc6c5)nc(-c5ccc(-c6ccccc6)c6ccccc56)n4)c4c5ccccc5oc4c3c2)cc1 ; c1ccc(-c2ccc3cc(-c4nc(-c5ccc(-c6cccc7ccccc67)cc5)nc(-c5ccc6ccccc6c5)n4)c4c5ccccc5oc4c3c2)cc1 ; c1ccc(-c2ccc3cc(-c4nc(-c5ccc6c(c5)oc5ccccc56)nc(-c5cccc6oc7ccccc7c56)n4)c4c5ccccc5oc4c3c2)cc1]

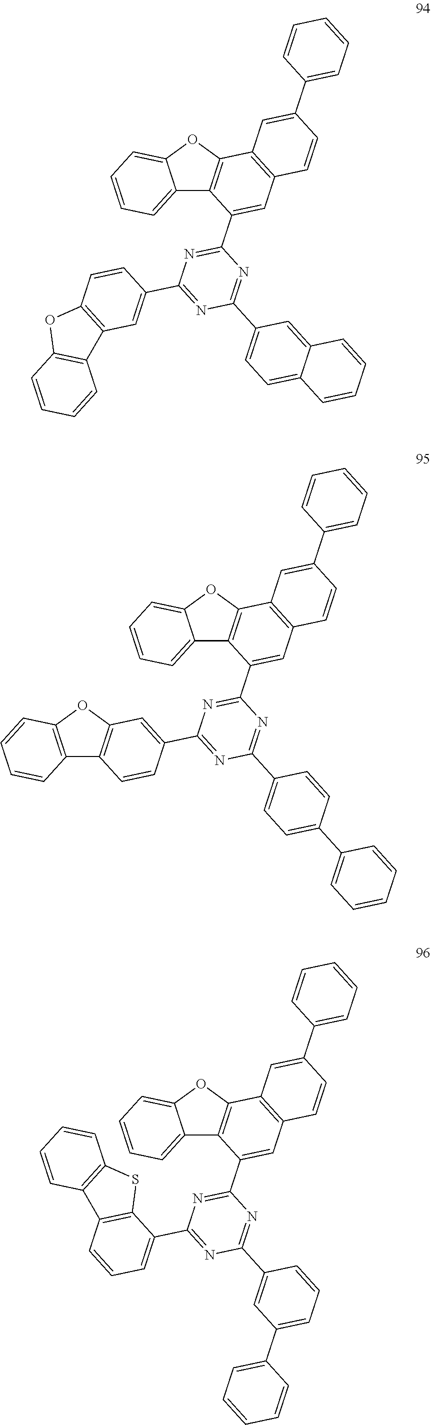 [SMILES: c1ccc(-c2ccc3cc(-c4nc(-c5ccc6ccccc6c5)nc(-c5ccc6oc7ccccc7c6c5)n4)c4c5ccccc5oc4c3c2)cc1 ; c1ccc(-c2ccc(-c3nc(-c4ccc5c(c4)oc4ccccc45)nc(-c4cc5ccc(-c6ccccc6)cc5c5oc6ccccc6c45)n3)cc2)cc1 ; c1ccc(-c2cccc(-c3nc(-c4cccc5c4sc4ccccc45)nc(-c4cc5ccc(-c6ccccc6)cc5c5oc6ccccc6c45)n3)c2)cc1]

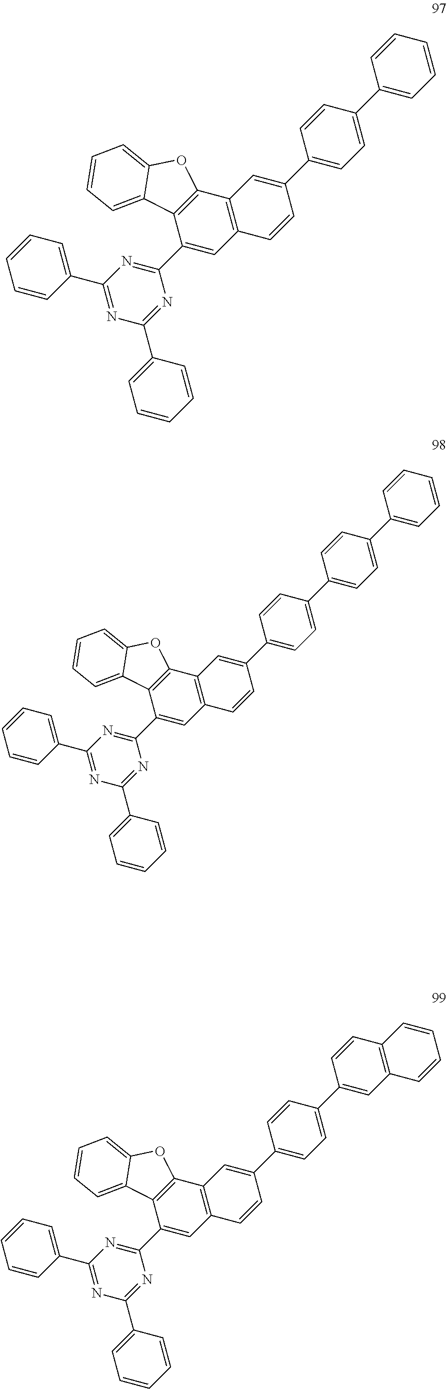 [SMILES: c1ccc(-c2ccc(-c3ccc4cc(-c5nc(-c6ccccc6)nc(-c6ccccc6)n5)c5c6ccccc6oc5c4c3)cc2)cc1 ; c1ccc(-c2ccc(-c3ccc(-c4ccc5cc(-c6nc(-c7ccccc7)nc(-c7ccccc7)n6)c6c7ccccc7oc6c5c4)cc3)cc2)cc1 ; c1ccc(-c2nc(-c3ccccc3)nc(-c3cc4ccc(-c5ccc(-c6ccc7ccccc7c6)cc5)cc4c4oc5ccccc5c34)n2)cc1]

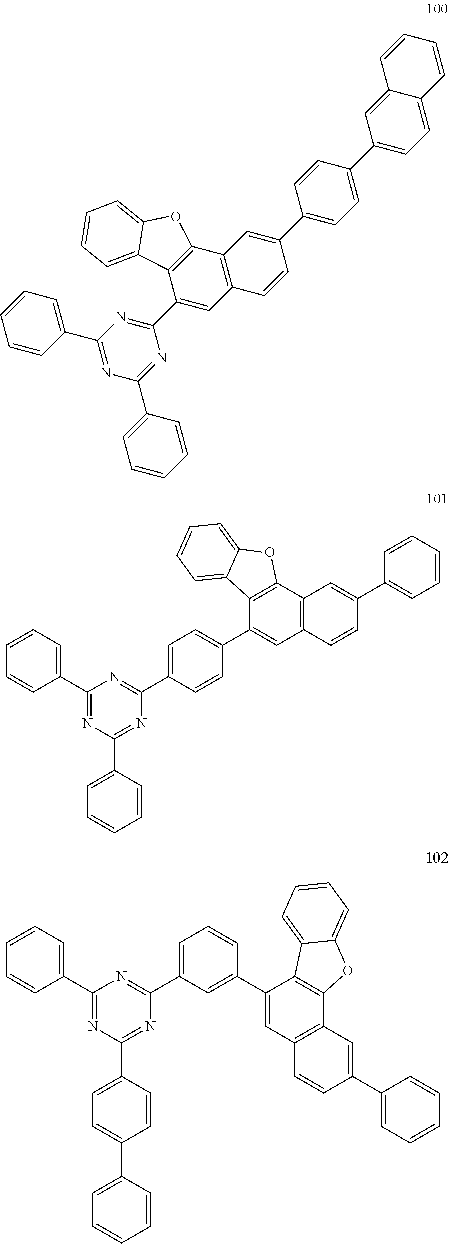 [SMILES: c1ccc(-c2nc(-c3ccccc3)nc(-c3cc4ccc(-c5ccc(-c6ccc7ccccc7c6)cc5)cc4c4oc5ccccc5c34)n2)cc1 ; c1ccc(-c2ccc3cc(-c4ccc(-c5nc(-c6ccccc6)nc(-c6ccccc6)n5)cc4)c4c5ccccc5oc4c3c2)cc1 ; c1ccc(-c2ccc(-c3nc(-c4ccccc4)nc(-c4cccc(-c5cc6ccc(-c7ccccc7)cc6c6oc7ccccc7c56)c4)n3)cc2)cc1]

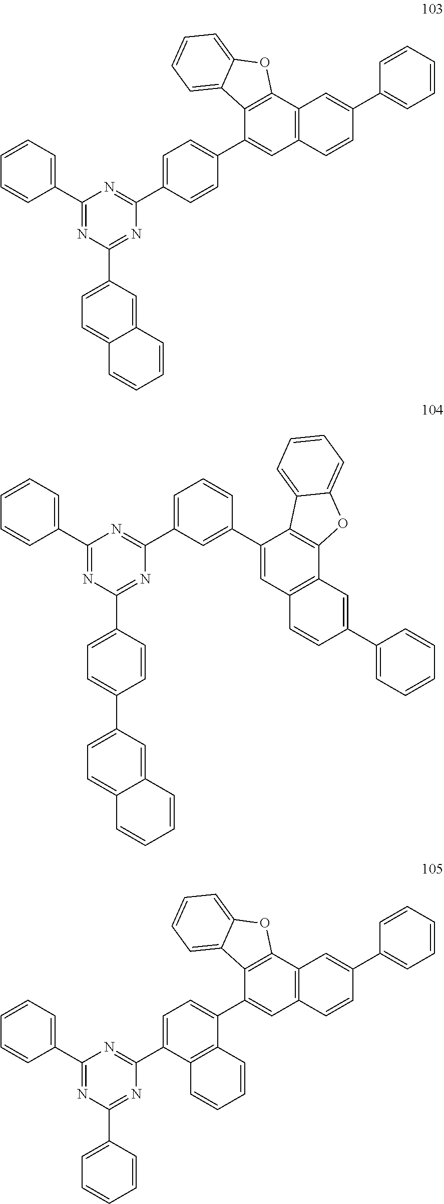 [SMILES: c1ccc(-c2ccc3cc(-c4ccc(-c5nc(-c6ccccc6)nc(-c6ccc7ccccc7c6)n5)cc4)c4c5ccccc5oc4c3c2)cc1 ; c1ccc(-c2ccc3cc(-c4cccc(-c5nc(-c6ccccc6)nc(-c6ccc(-c7ccc8ccccc8c7)cc6)n5)c4)c4c5ccccc5oc4c3c2)cc1 ; c1ccc(-c2ccc3cc(-c4ccc(-c5nc(-c6ccccc6)nc(-c6ccccc6)n5)c5ccccc45)c4c5ccccc5oc4c3c2)cc1]

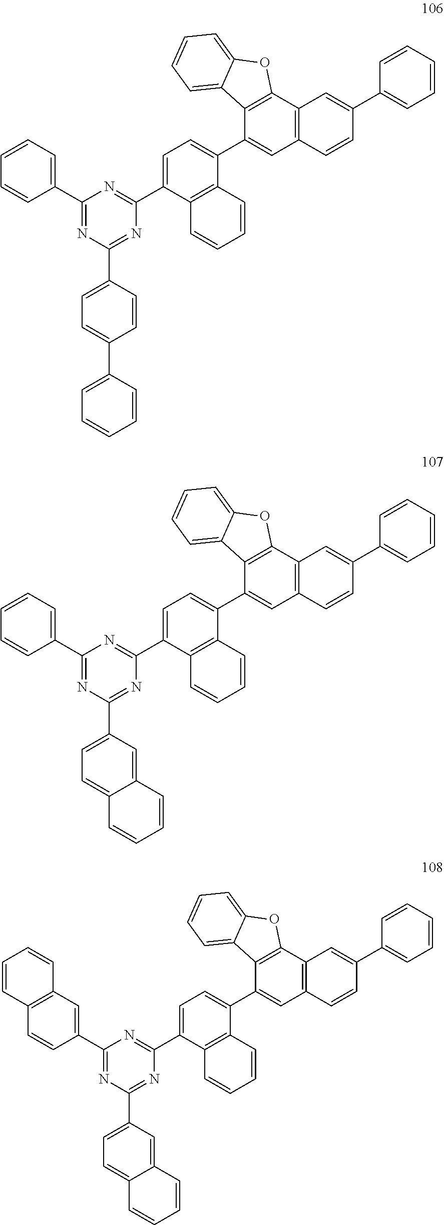 [SMILES: c1ccc(-c2ccc(-c3nc(-c4ccccc4)nc(-c4ccc(-c5cc6ccc(-c7ccccc7)cc6c6oc7ccccc7c56)c5ccccc45)n3)cc2)cc1 ; c1ccc(-c2ccc3cc(-c4ccc(-c5nc(-c6ccccc6)nc(-c6ccc7ccccc7c6)n5)c5ccccc45)c4c5ccccc5oc4c3c2)cc1 ; c1ccc(-c2ccc3cc(-c4ccc(-c5nc(-c6ccc7ccccc7c6)nc(-c6ccc7ccccc7c6)n5)c5ccccc45)c4c5ccccc5oc4c3c2)cc1]

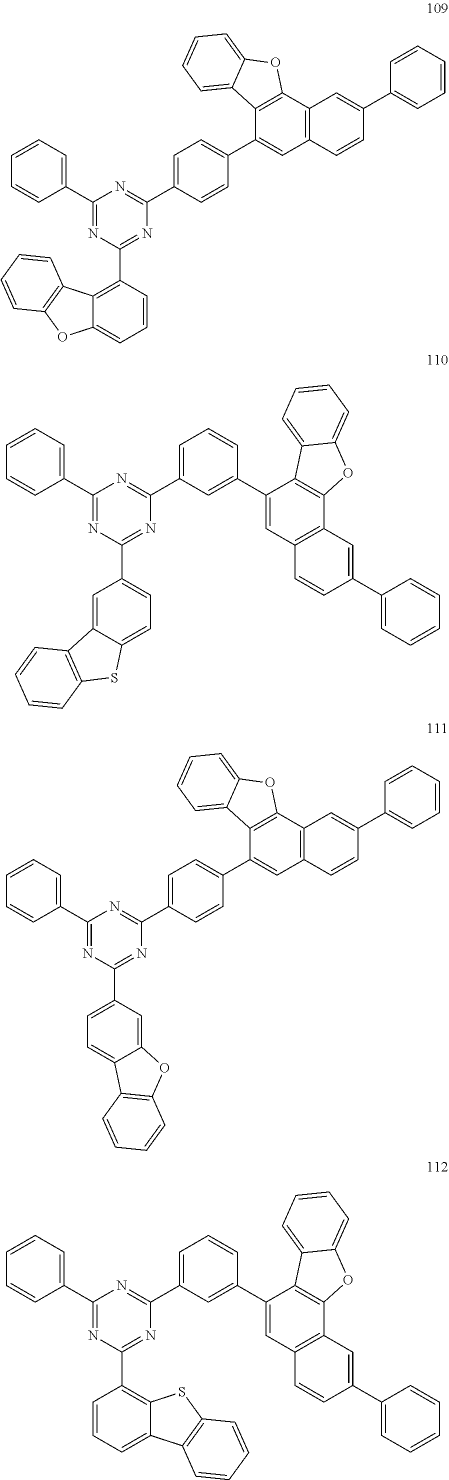 [SMILES: c1ccc(-c2ccc3cc(-c4ccc(-c5nc(-c6ccccc6)nc(-c6cccc7oc8ccccc8c67)n5)cc4)c4c5ccccc5oc4c3c2)cc1 ; c1ccc(-c2ccc3cc(-c4cccc(-c5nc(-c6ccccc6)nc(-c6ccc7sc8ccccc8c7c6)n5)c4)c4c5ccccc5oc4c3c2)cc1 ; c1ccc(-c2ccc3cc(-c4ccc(-c5nc(-c6ccccc6)nc(-c6ccc7c(c6)oc6ccccc67)n5)cc4)c4c5ccccc5oc4c3c2)cc1 ; c1ccc(-c2ccc3cc(-c4cccc(-c5nc(-c6ccccc6)nc(-c6cccc7c6sc6ccccc67)n5)c4)c4c5ccccc5oc4c3c2)cc1]

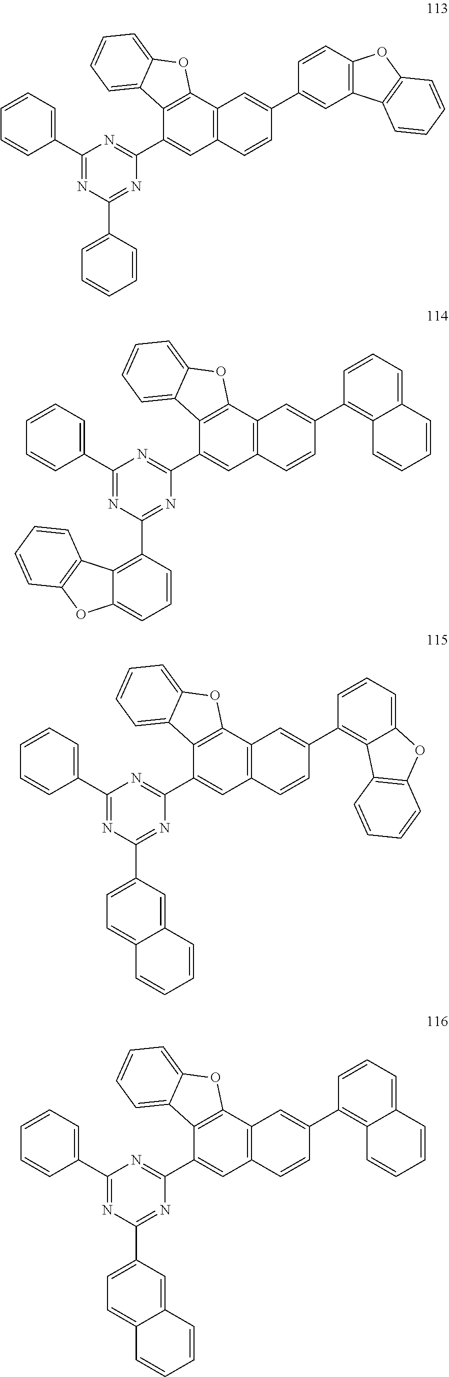 [SMILES: c1ccc(-c2nc(-c3ccccc3)nc(-c3cc4ccc(-c5ccc6oc7ccccc7c6c5)cc4c4oc5ccccc5c34)n2)cc1 ; c1ccc(-c2nc(-c3cccc4oc5ccccc5c34)nc(-c3cc4ccc(-c5cccc6ccccc56)cc4c4oc5ccccc5c34)n2)cc1 ; c1ccc(-c2nc(-c3ccc4ccccc4c3)nc(-c3cc4ccc(-c5cccc6oc7ccccc7c56)cc4c4oc5ccccc5c34)n2)cc1 ; c1ccc(-c2nc(-c3ccc4ccccc4c3)nc(-c3cc4ccc(-c5cccc6ccccc56)cc4c4oc5ccccc5c34)n2)cc1]

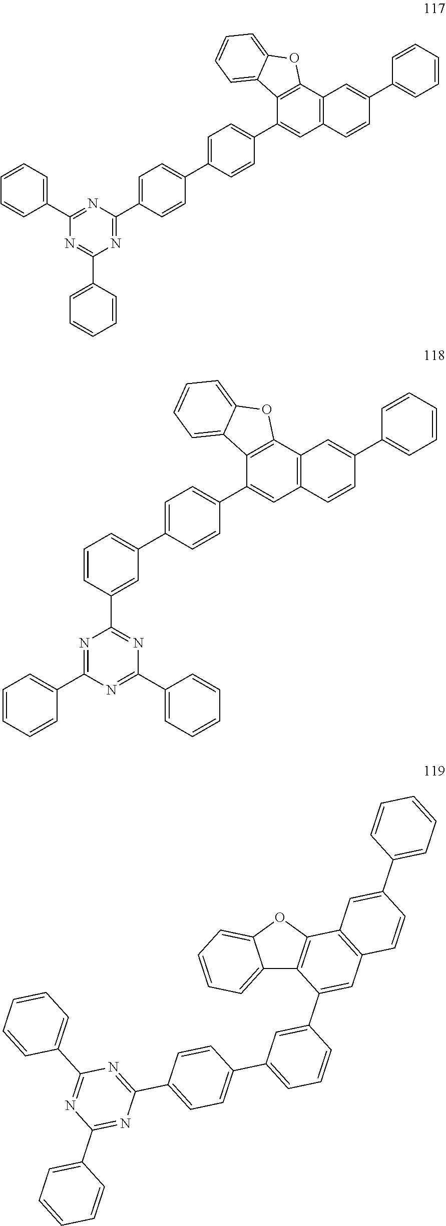 [SMILES: c1ccc(-c2ccc3cc(-c4ccc(-c5ccc(-c6nc(-c7ccccc7)nc(-c7ccccc7)n6)cc5)cc4)c4c5ccccc5oc4c3c2)cc1 ; c1ccc(-c2ccc3cc(-c4ccc(-c5cccc(-c6nc(-c7ccccc7)nc(-c7ccccc7)n6)c5)cc4)c4c5ccccc5oc4c3c2)cc1 ; c1ccc(-c2ccc3cc(-c4cccc(-c5ccc(-c6nc(-c7ccccc7)nc(-c7ccccc7)n6)cc5)c4)c4c5ccccc5oc4c3c2)cc1]

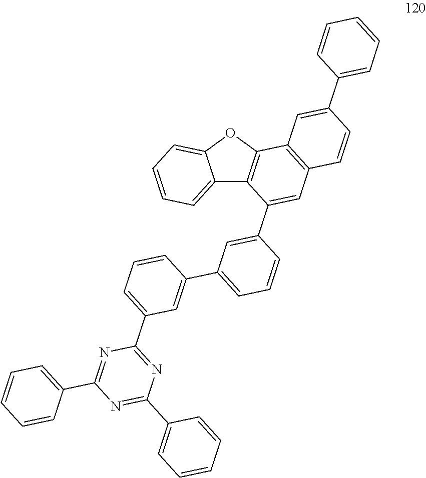 [SMILES: c1ccc(-c2ccc3cc(-c4cccc(-c5cccc(-c6nc(-c7ccccc7)nc(-c7ccccc7)n6)c5)c4)c4c5ccccc5oc4c3c2)cc1]

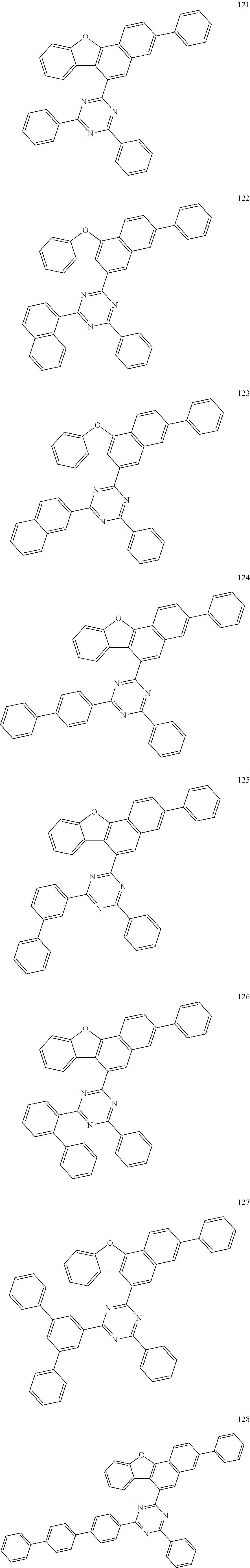

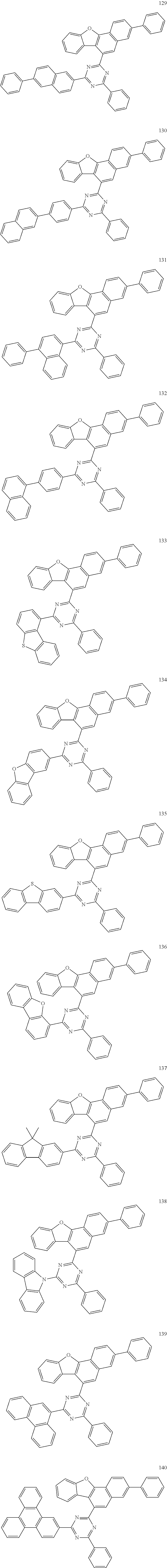

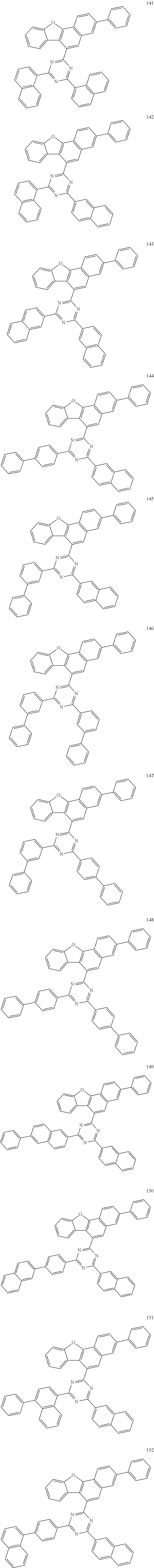

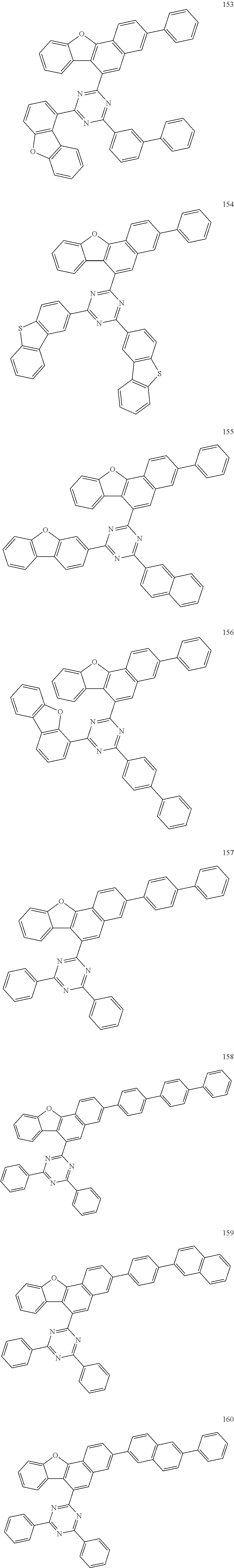

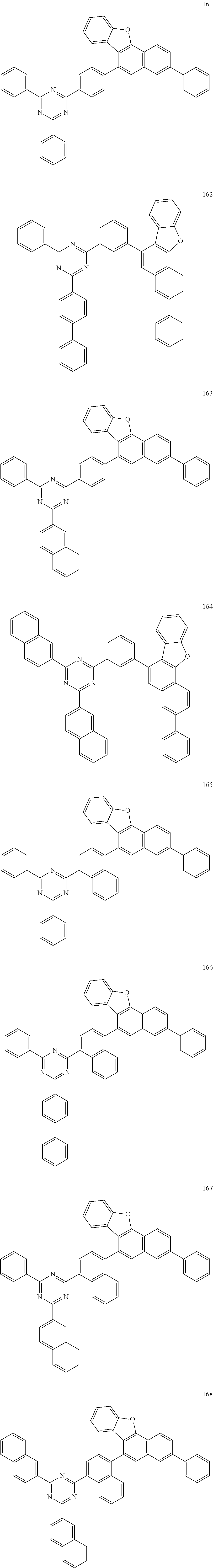

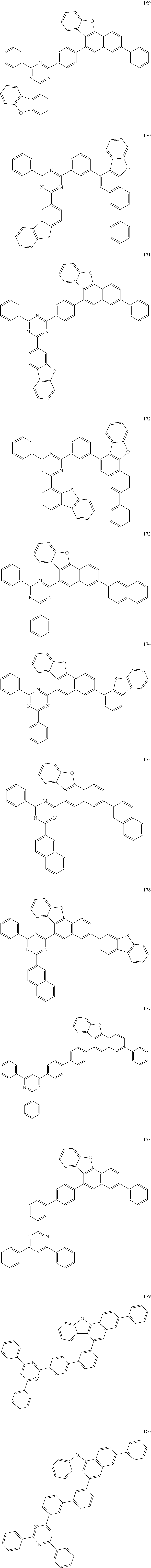

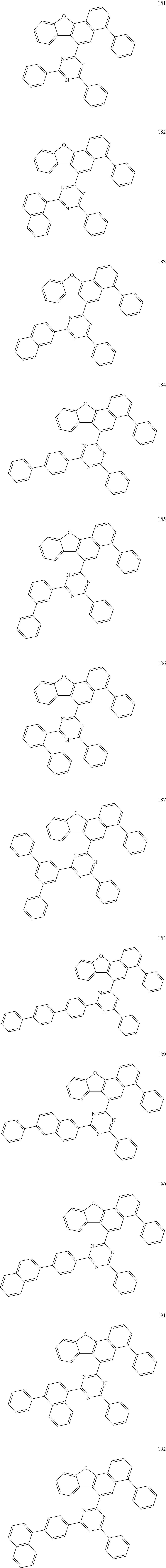

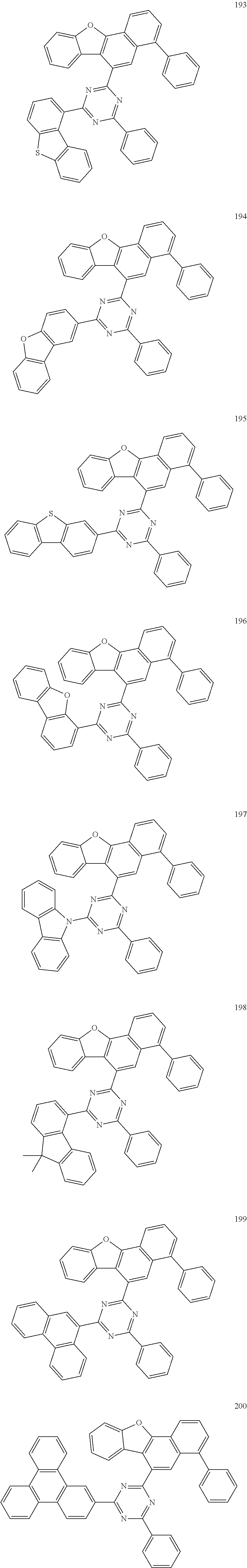

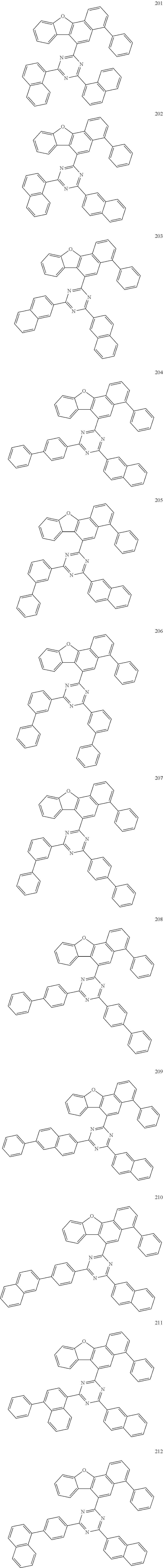

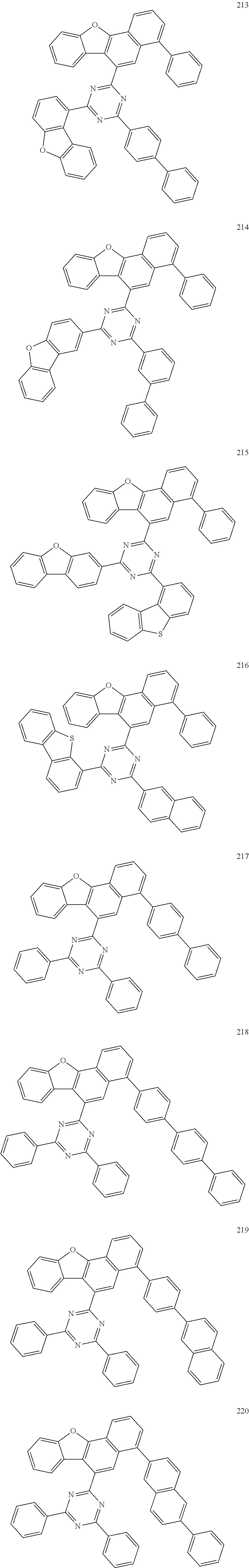

[chemical structure]

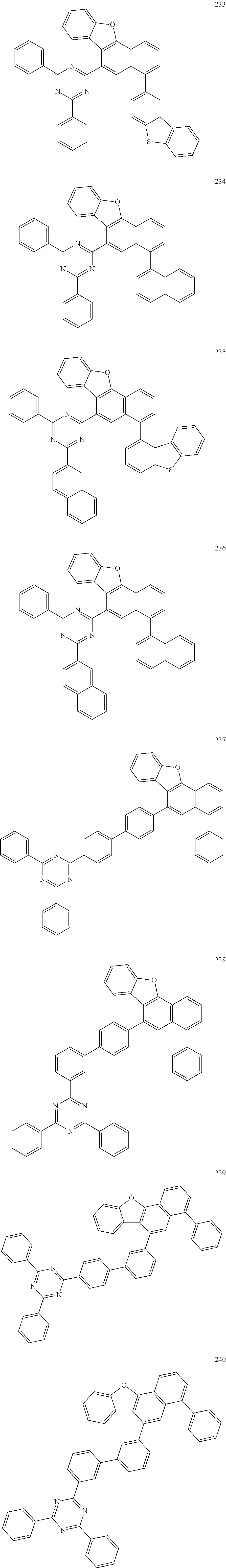

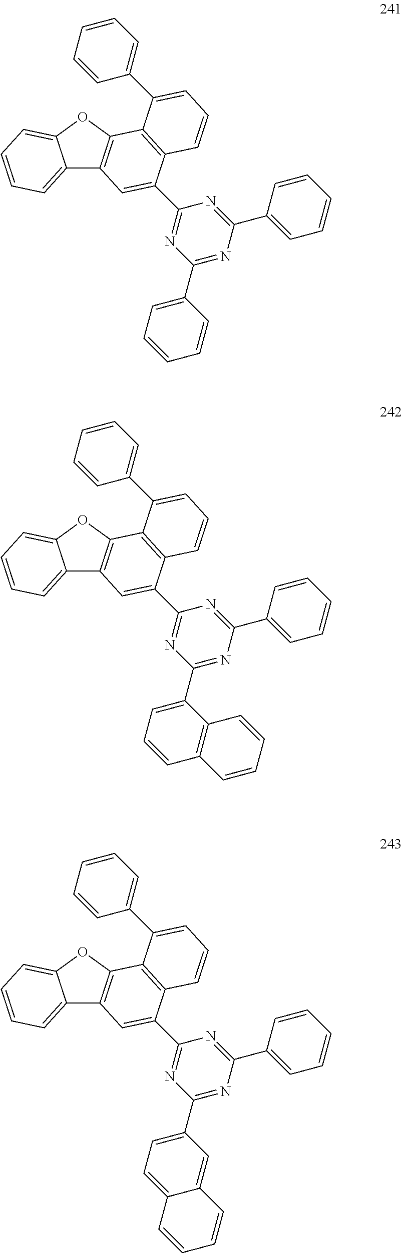 [SMILES: c1ccc(-c2nc(-c3ccccc3)nc(-c3cc4c5ccccc5oc4c4c(-c5ccccc5)cccc34)n2)cc1 ; c1ccc(-c2nc(-c3cccc4ccccc34)nc(-c3cc4c5ccccc5oc4c4c(-c5ccccc5)cccc34)n2)cc1 ; c1ccc(-c2nc(-c3ccc4ccccc4c3)nc(-c3cc4c5ccccc5oc4c4c(-c5ccccc5)cccc34)n2)cc1]

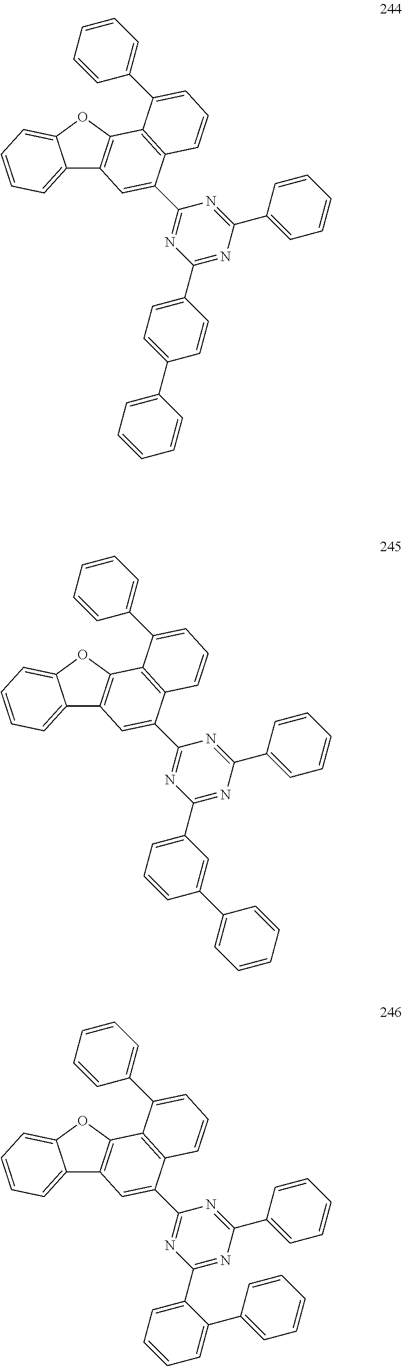 [SMILES: c1ccc(-c2ccc(-c3nc(-c4ccccc4)nc(-c4cc5c6ccccc6oc5c5c(-c6ccccc6)cccc45)n3)cc2)cc1 ; c1ccc(-c2cccc(-c3nc(-c4ccccc4)nc(-c4cc5c6ccccc6oc5c5c(-c6ccccc6)cccc45)n3)c2)cc1 ; c1ccc(-c2nc(-c3ccccc3-c3ccccc3)nc(-c3cc4c5ccccc5oc4c4c(-c5ccccc5)cccc34)n2)cc1]

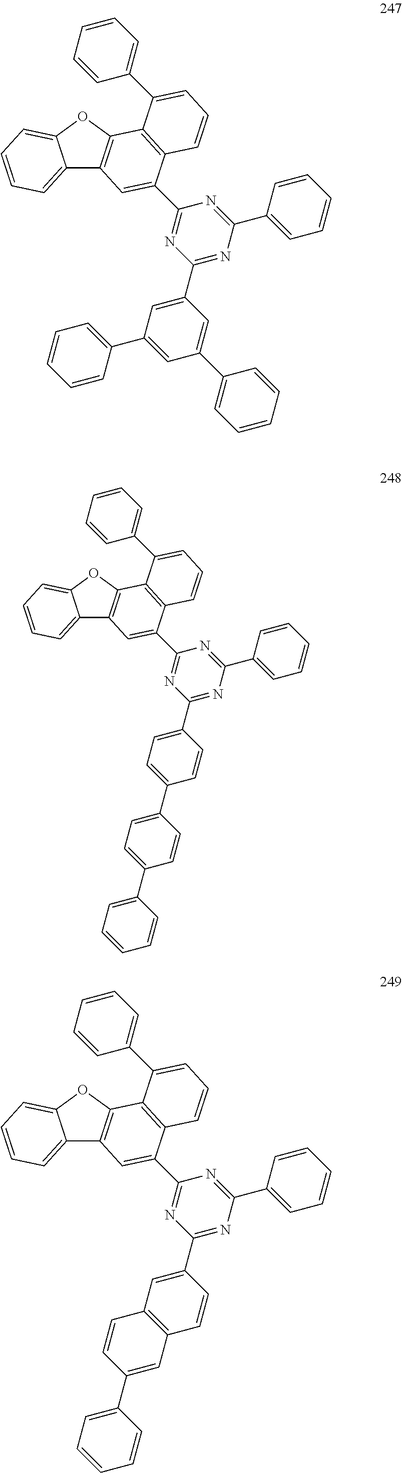

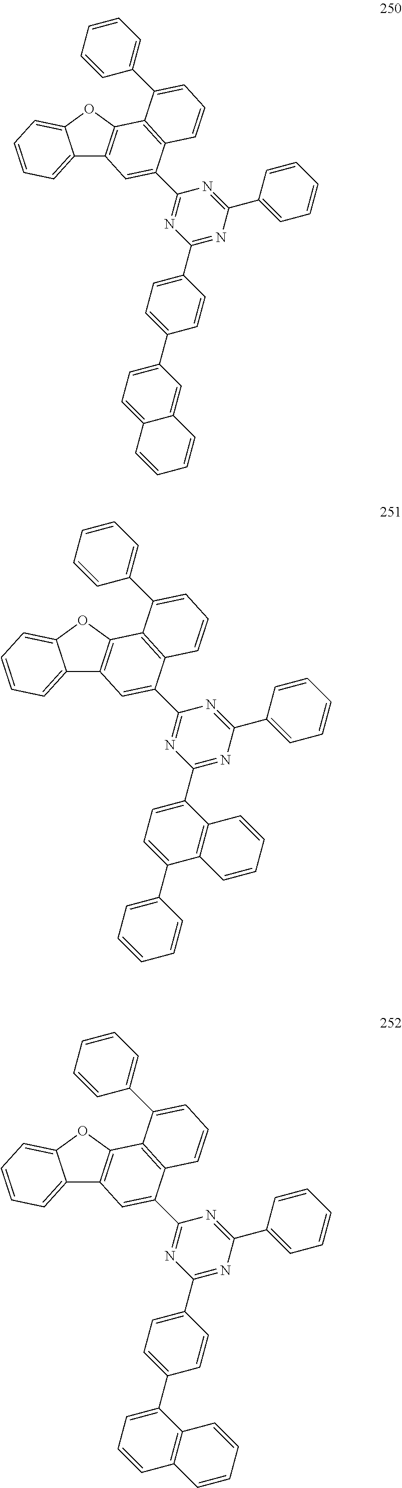 [SMILES: c1ccc(-c2nc(-c3ccc(-c4ccc5ccccc5c4)cc3)nc(-c3cc4c5ccccc5oc4c4c(-c5ccccc5)cccc34)n2)cc1 ; c1ccc(-c2nc(-c3ccc(-c4ccccc4)c4ccccc34)nc(-c3cc4c5ccccc5oc4c4c(-c5ccccc5)cccc34)n2)cc1 ; c1ccc(-c2nc(-c3ccc(-c4cccc5ccccc45)cc3)nc(-c3cc4c5ccccc5oc4c4c(-c5ccccc5)cccc34)n2)cc1]

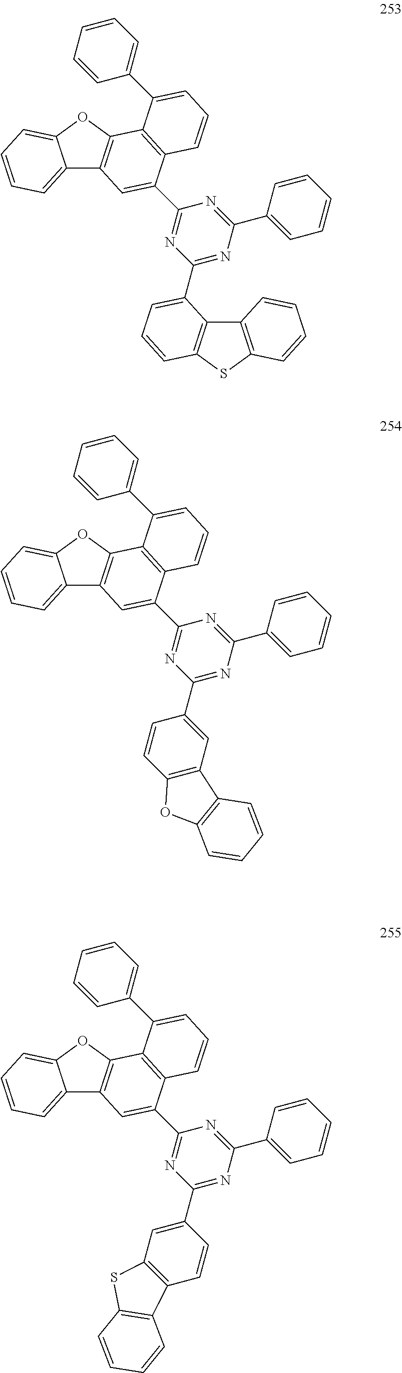 [SMILES: c1ccc(-c2nc(-c3cc4c5ccccc5oc4c4c(-c5ccccc5)cccc34)nc(-c3cccc4sc5ccccc5c34)n2)cc1 ; c1ccc(-c2nc(-c3ccc4oc5ccccc5c4c3)nc(-c3cc4c5ccccc5oc4c4c(-c5ccccc5)cccc34)n2)cc1 ; c1ccc(-c2nc(-c3ccc4c(c3)sc3ccccc34)nc(-c3cc4c5ccccc5oc4c4c(-c5ccccc5)cccc34)n2)cc1]

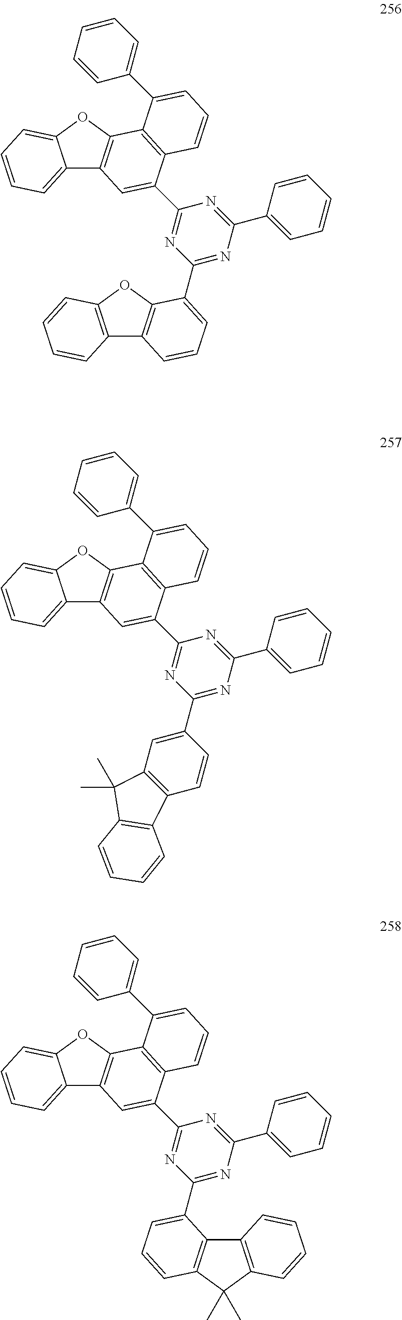 [SMILES: c1ccc(-c2nc(-c3cc4c5ccccc5oc4c4c(-c5ccccc5)cccc34)nc(-c3cccc4c3oc3ccccc34)n2)cc1 ; CC1(C)c2ccccc2-c2ccc(-c3nc(-c4ccccc4)nc(-c4cc5c6ccccc6oc5c5c(-c6ccccc6)cccc45)n3)cc21 ; CC1(C)c2ccccc2-c2c(-c3nc(-c4ccccc4)nc(-c4cc5c6ccccc6oc5c5c(-c6ccccc6)cccc45)n3)cccc21]

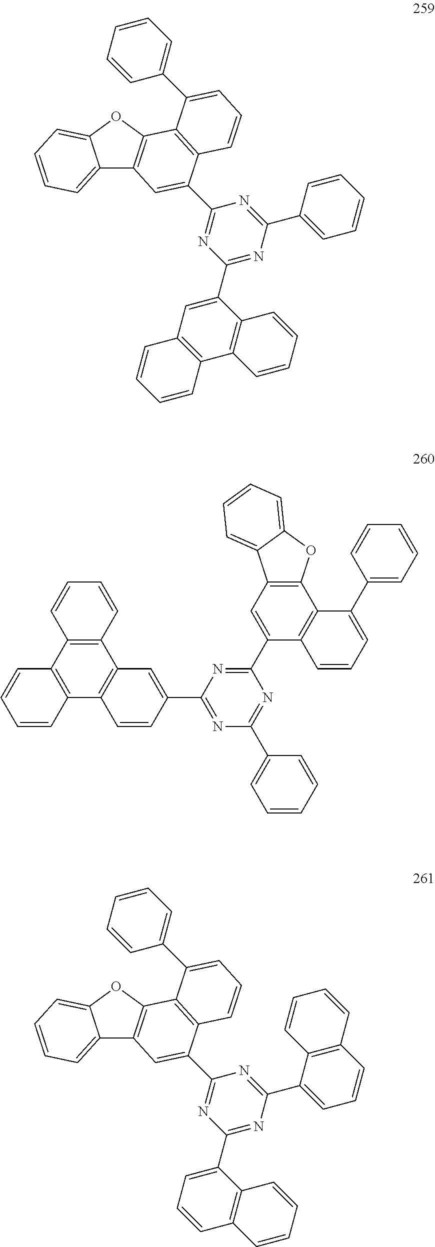 [SMILES: c1ccc(-c2nc(-c3cc4ccccc4c4ccccc34)nc(-c3cc4c5ccccc5oc4c4c(-c5ccccc5)cccc34)n2)cc1 ; c1ccc(-c2nc(-c3ccc4c5ccccc5c5ccccc5c4c3)nc(-c3cc4c5ccccc5oc4c4c(-c5ccccc5)cccc34)n2)cc1 ; c1ccc(-c2cccc3c(-c4nc(-c5cccc6ccccc56)nc(-c5cccc6ccccc56)n4)cc4c5ccccc5oc4c23)cc1]

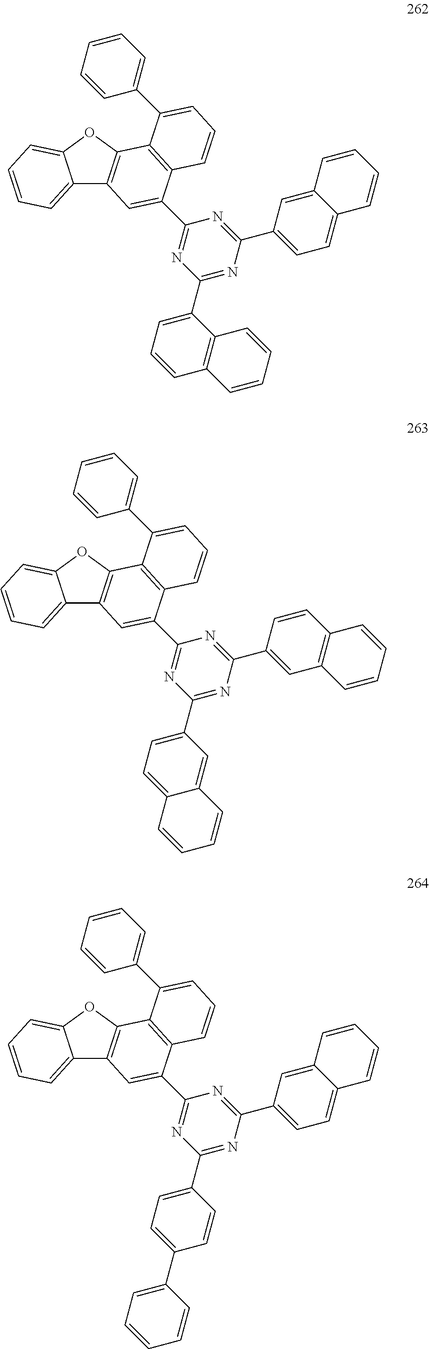 [SMILES: c1ccc(-c2cccc3c(-c4nc(-c5ccc6ccccc6c5)nc(-c5cccc6ccccc56)n4)cc4c5ccccc5oc4c23)cc1 ; c1ccc(-c2cccc3c(-c4nc(-c5ccc6ccccc6c5)nc(-c5ccc6ccccc6c5)n4)cc4c5ccccc5oc4c23)cc1 ; c1ccc(-c2ccc(-c3nc(-c4ccc5ccccc5c4)nc(-c4cc5c6ccccc6oc5c5c(-c6ccccc6)cccc45)n3)cc2)cc1]

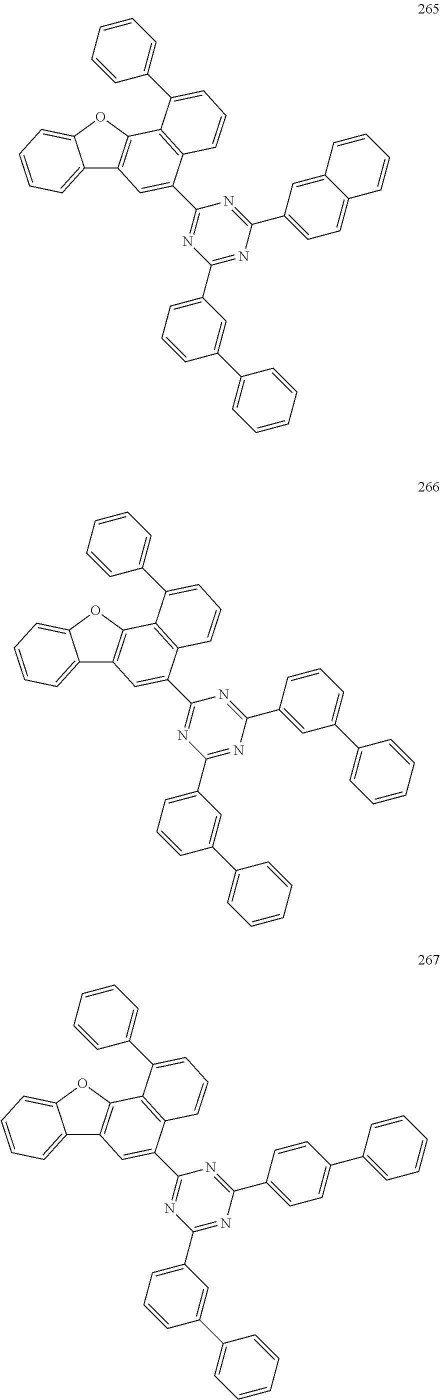 [SMILES: c1ccc(-c2cccc(-c3nc(-c4ccc5ccccc5c4)nc(-c4cc5c6ccccc6oc5c5c(-c6ccccc6)cccc45)n3)c2)cc1 ; c1ccc(-c2cccc(-c3nc(-c4cccc(-c5ccccc5)c4)nc(-c4cc5c6ccccc6oc5c5c(-c6ccccc6)cccc45)n3)c2)cc1 ; c1ccc(-c2ccc(-c3nc(-c4cccc(-c5ccccc5)c4)nc(-c4cc5c6ccccc6oc5c5c(-c6ccccc6)cccc45)n3)cc2)cc1]

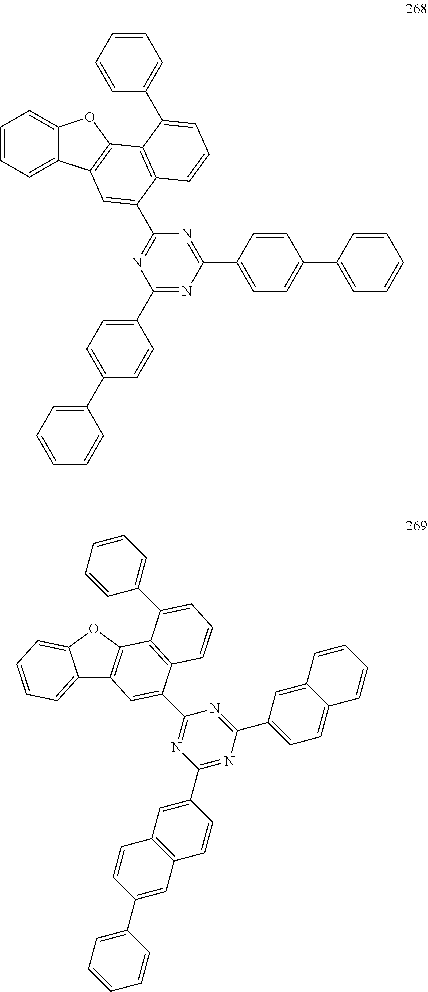 [SMILES: c1ccc(-c2ccc(-c3nc(-c4ccc(-c5ccccc5)cc4)nc(-c4cc5c6ccccc6oc5c5c(-c6ccccc6)cccc45)n3)cc2)cc1 ; c1ccc(-c2ccc3cc(-c4nc(-c5ccc6ccccc6c5)nc(-c5cc6c7ccccc7oc6c6c(-c7ccccc7)cccc56)n4)ccc3c2)cc1]

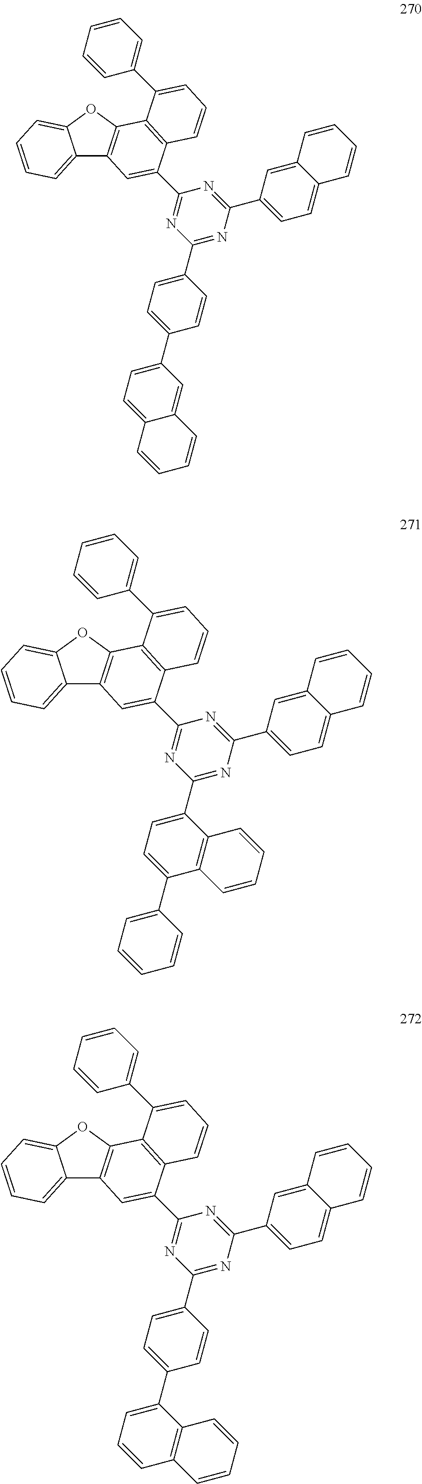 [SMILES: c1ccc(-c2cccc3c(-c4nc(-c5ccc(-c6ccc7ccccc7c6)cc5)nc(-c5ccc6ccccc6c5)n4)cc4c5ccccc5oc4c23)cc1 ; c1ccc(-c2ccc(-c3nc(-c4ccc5ccccc5c4)nc(-c4cc5c6ccccc6oc5c5c(-c6ccccc6)cccc45)n3)c3ccccc23)cc1 ; c1ccc(-c2cccc3c(-c4nc(-c5ccc(-c6cccc7ccccc67)cc5)nc(-c5ccc6ccccc6c5)n4)cc4c5ccccc5oc4c23)cc1]

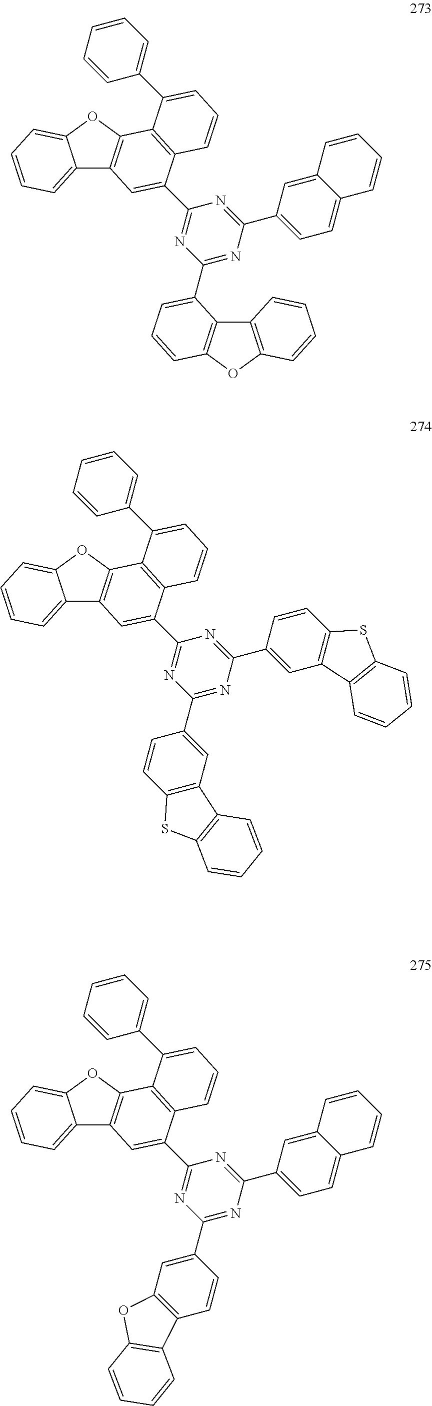 [SMILES: c1ccc(-c2cccc3c(-c4nc(-c5ccc6ccccc6c5)nc(-c5cccc6oc7ccccc7c56)n4)cc4c5ccccc5oc4c23)cc1 ; c1ccc(-c2cccc3c(-c4nc(-c5ccc6sc7ccccc7c6c5)nc(-c5ccc6sc7ccccc7c6c5)n4)cc4c5ccccc5oc4c23)cc1 ; c1ccc(-c2cccc3c(-c4nc(-c5ccc6ccccc6c5)nc(-c5ccc6c(c5)oc5ccccc56)n4)cc4c5ccccc5oc4c23)cc1]

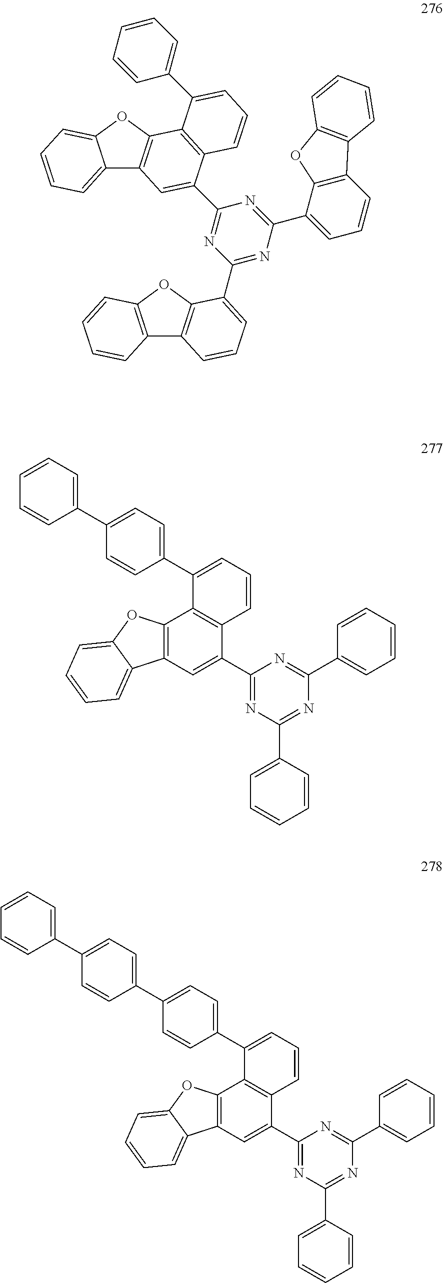 [SMILES: c1ccc(-c2cccc3c(-c4nc(-c5cccc6c5oc5ccccc56)nc(-c5cccc6c5oc5ccccc56)n4)cc4c5ccccc5oc4c23)cc1 ; c1ccc(-c2ccc(-c3cccc4c(-c5nc(-c6ccccc6)nc(-c6ccccc6)n5)cc5c6ccccc6oc5c34)cc2)cc1 ; c1ccc(-c2ccc(-c3ccc(-c4cccc5c(-c6nc(-c7ccccc7)nc(-c7ccccc7)n6)cc6c7ccccc7oc6c45)cc3)cc2)cc1]

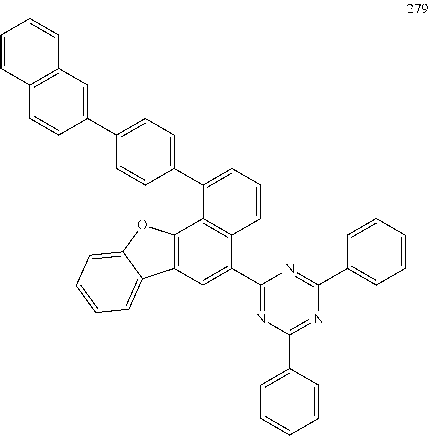 [SMILES: c1ccc(-c2nc(-c3ccccc3)nc(-c3cc4c5ccccc5oc4c4c(-c5ccc(-c6ccc7ccccc7c6)cc5)cccc34)n2)cc1]

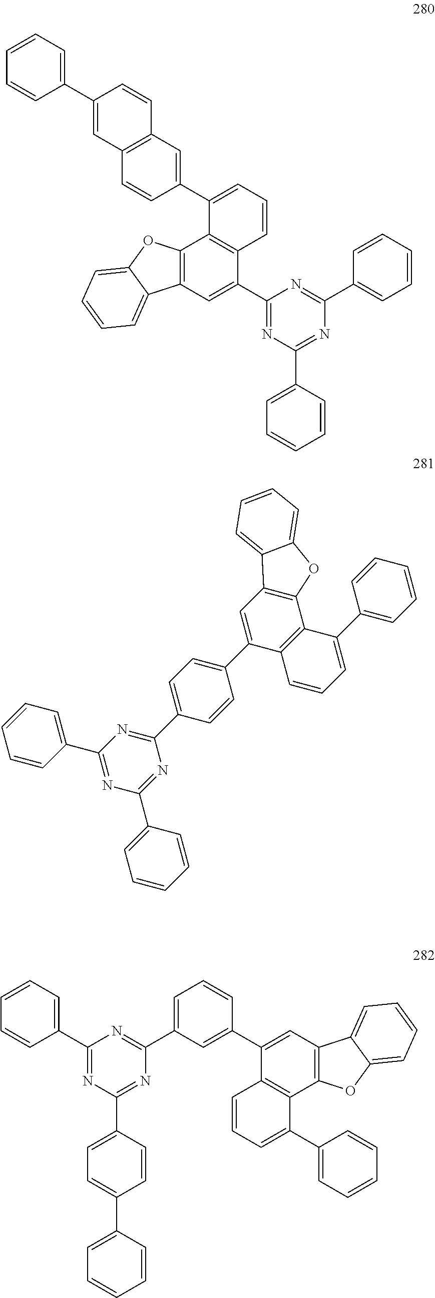

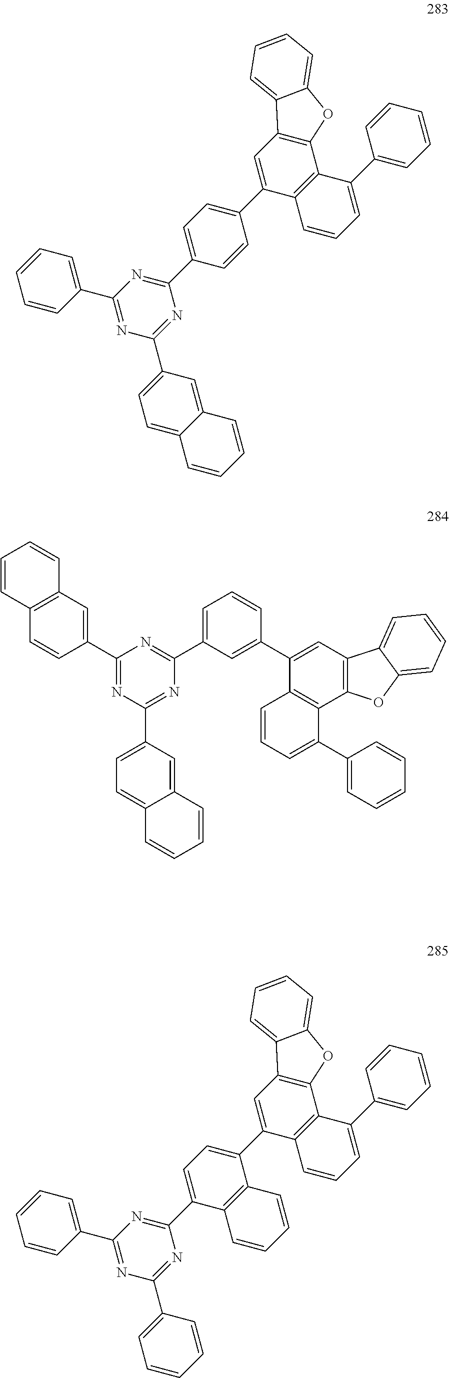 [SMILES: c1ccc(-c2nc(-c3ccc(-c4cc5c6ccccc6oc5c5c(-c6ccccc6)cccc45)cc3)nc(-c3ccc4ccccc4c3)n2)cc1 ; c1ccc(-c2cccc3c(-c4cccc(-c5nc(-c6ccc7ccccc7c6)nc(-c6ccc7ccccc7c6)n5)c4)cc4c5ccccc5oc4c23)cc1 ; c1ccc(-c2nc(-c3ccccc3)nc(-c3ccc(-c4cc5c6ccccc6oc5c5c(-c6ccccc6)cccc45)c4ccccc34)n2)cc1]

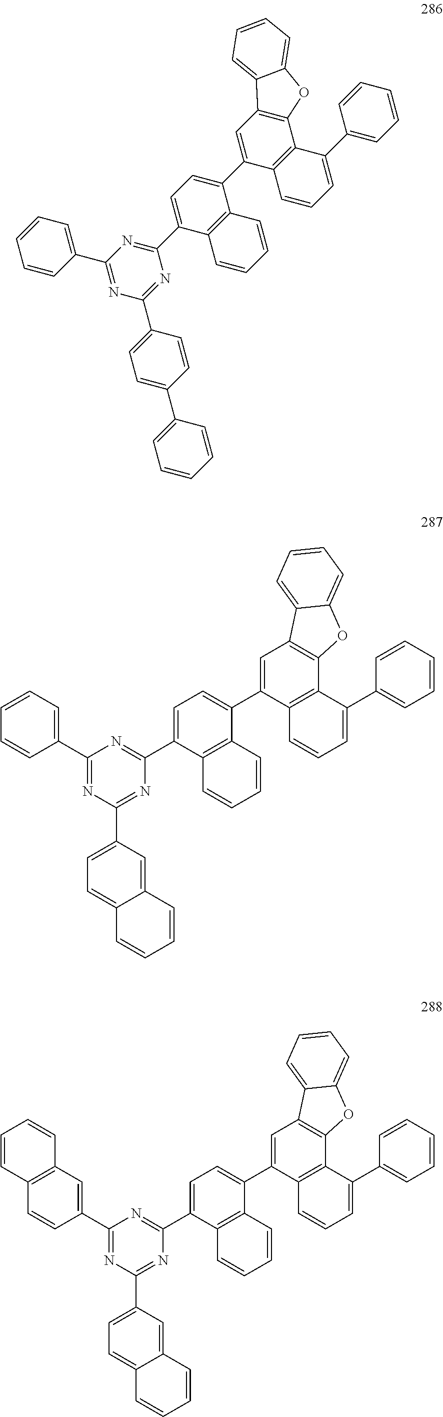 [SMILES: c1ccc(-c2ccc(-c3nc(-c4ccccc4)nc(-c4ccc(-c5cc6c7ccccc7oc6c6c(-c7ccccc7)cccc56)c5ccccc45)n3)cc2)cc1 ; c1ccc(-c2nc(-c3ccc4ccccc4c3)nc(-c3ccc(-c4cc5c6ccccc6oc5c5c(-c6ccccc6)cccc45)c4ccccc34)n2)cc1 ; c1ccc(-c2cccc3c(-c4ccc(-c5nc(-c6ccc7ccccc7c6)nc(-c6ccc7ccccc7c6)n5)c5ccccc45)cc4c5ccccc5oc4c23)cc1]

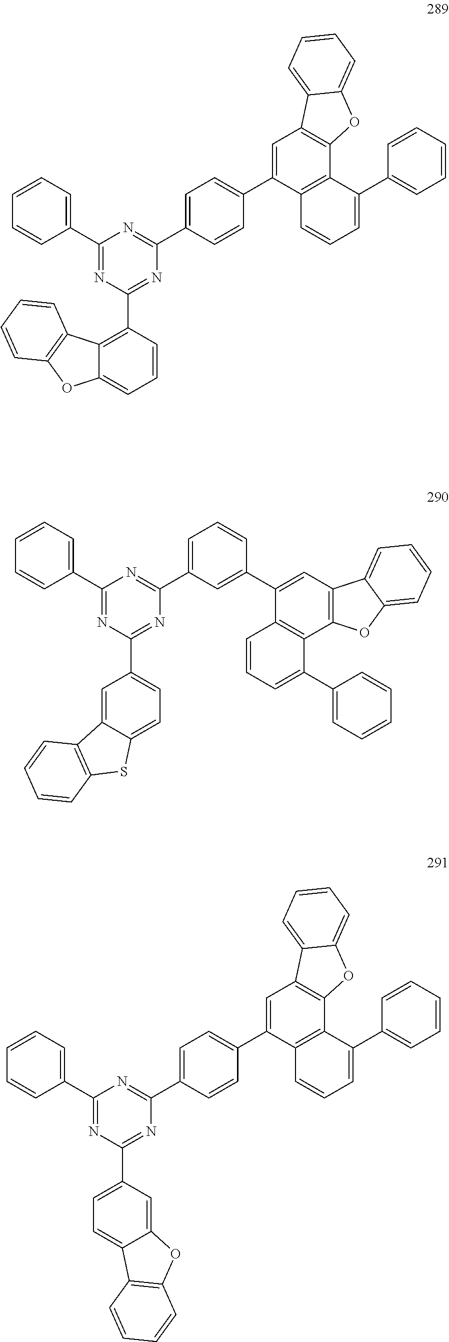 [SMILES: c1ccc(-c2nc(-c3ccc(-c4cc5c6ccccc6oc5c5c(-c6ccccc6)cccc45)cc3)nc(-c3cccc4oc5ccccc5c34)n2)cc1 ; c1ccc(-c2nc(-c3cccc(-c4cc5c6ccccc6oc5c5c(-c6ccccc6)cccc45)c3)nc(-c3ccc4sc5ccccc5c4c3)n2)cc1 ; c1ccc(-c2nc(-c3ccc(-c4cc5c6ccccc6oc5c5c(-c6ccccc6)cccc45)cc3)nc(-c3ccc4c(c3)oc3ccccc34)n2)cc1]

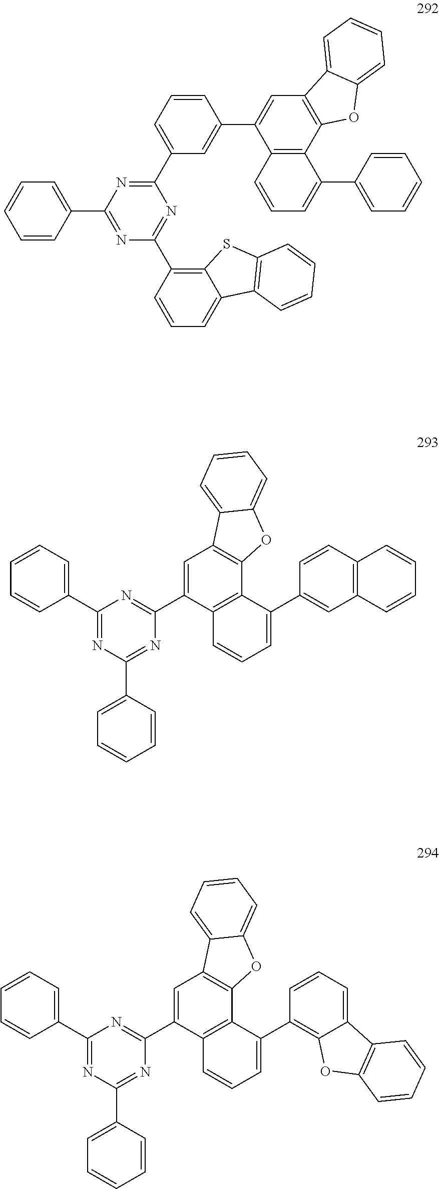 [SMILES: c1ccc(-c2nc(-c3cccc(-c4cc5c6ccccc6oc5c5c(-c6ccccc6)cccc45)c3)nc(-c3cccc4c3sc3ccccc34)n2)cc1 ; c1ccc(-c2nc(-c3ccccc3)nc(-c3cc4c5ccccc5oc4c4c(-c5ccc6ccccc6c5)cccc34)n2)cc1 ; c1ccc(-c2nc(-c3ccccc3)nc(-c3cc4c5ccccc5oc4c4c(-c5cccc6c5oc5ccccc56)cccc34)n2)cc1]

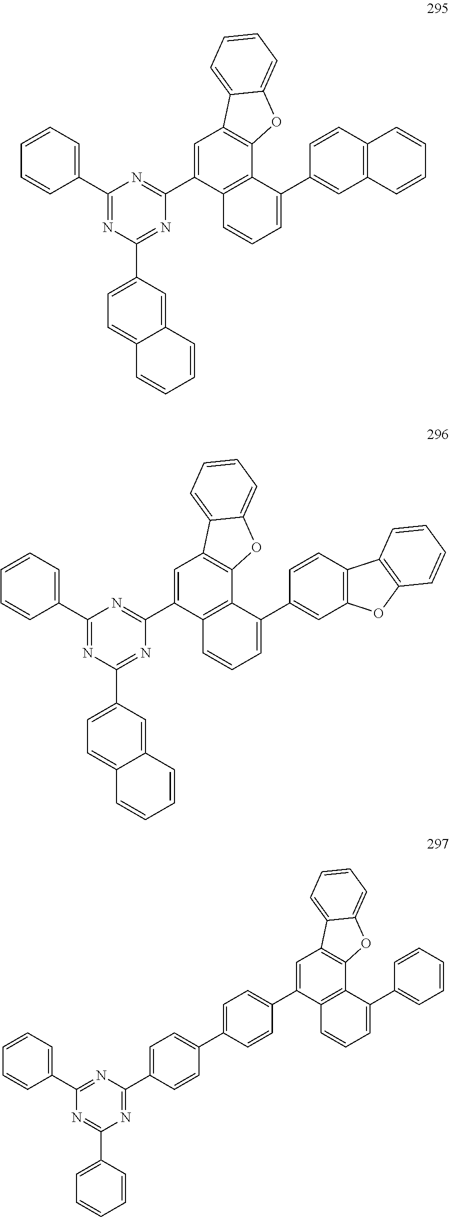 [SMILES: c1ccc(-c2nc(-c3ccc4ccccc4c3)nc(-c3cc4c5ccccc5oc4c4c(-c5ccc6ccccc6c5)cccc34)n2)cc1 ; c1ccc(-c2nc(-c3ccc4ccccc4c3)nc(-c3cc4c5ccccc5oc4c4c(-c5ccc6c(c5)oc5ccccc56)cccc34)n2)cc1 ; c1ccc(-c2nc(-c3ccccc3)nc(-c3ccc(-c4ccc(-c5cc6c7ccccc7oc6c6c(-c7ccccc7)cccc56)cc4)cc3)n2)cc1]

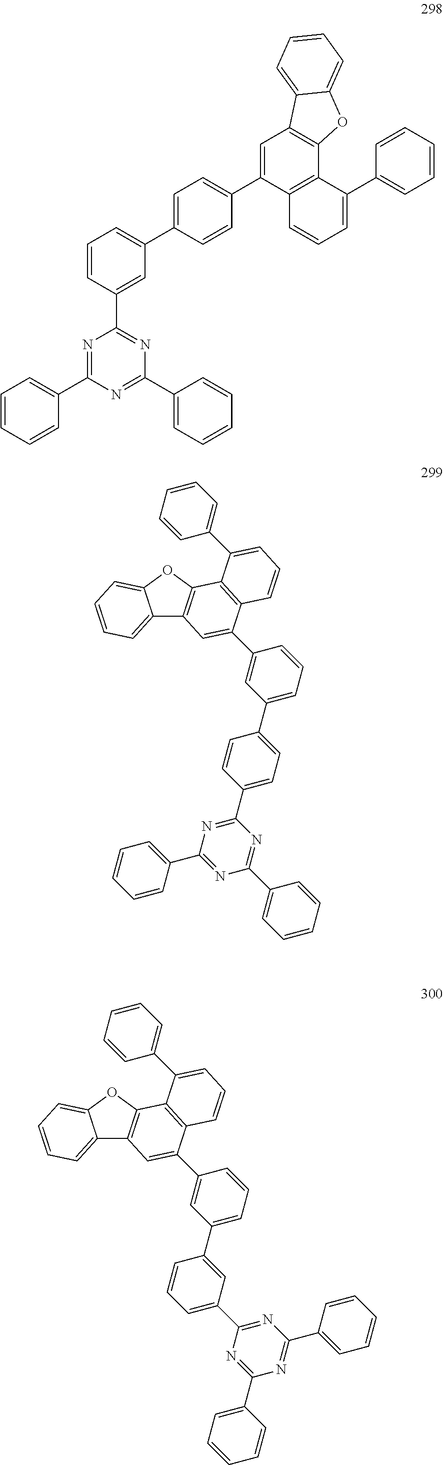

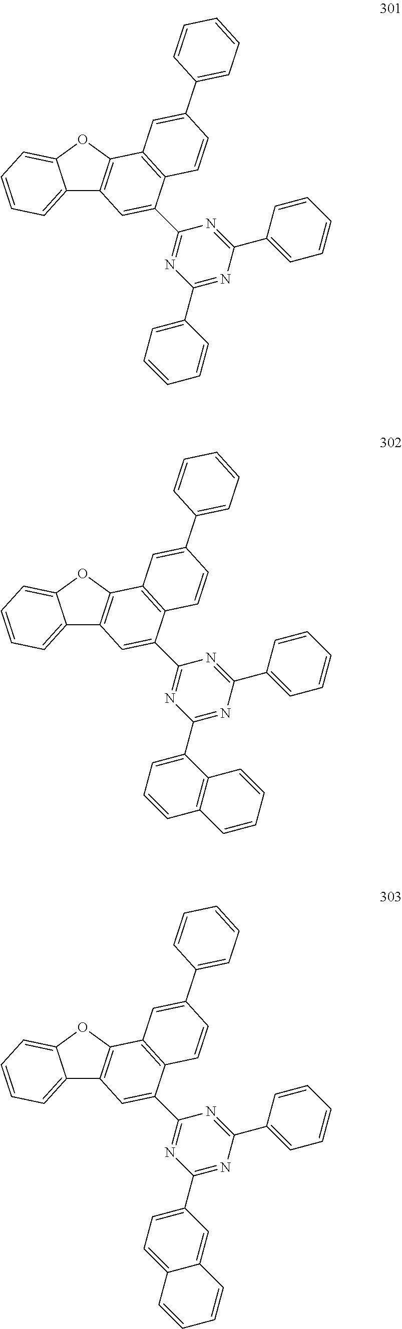 [SMILES: c1ccc(-c2ccc3c(-c4nc(-c5ccccc5)nc(-c5ccccc5)n4)cc4c5ccccc5oc4c3c2)cc1 ; c1ccc(-c2ccc3c(-c4nc(-c5ccccc5)nc(-c5cccc6ccccc56)n4)cc4c5ccccc5oc4c3c2)cc1 ; c1ccc(-c2ccc3c(-c4nc(-c5ccccc5)nc(-c5ccc6ccccc6c5)n4)cc4c5ccccc5oc4c3c2)cc1]

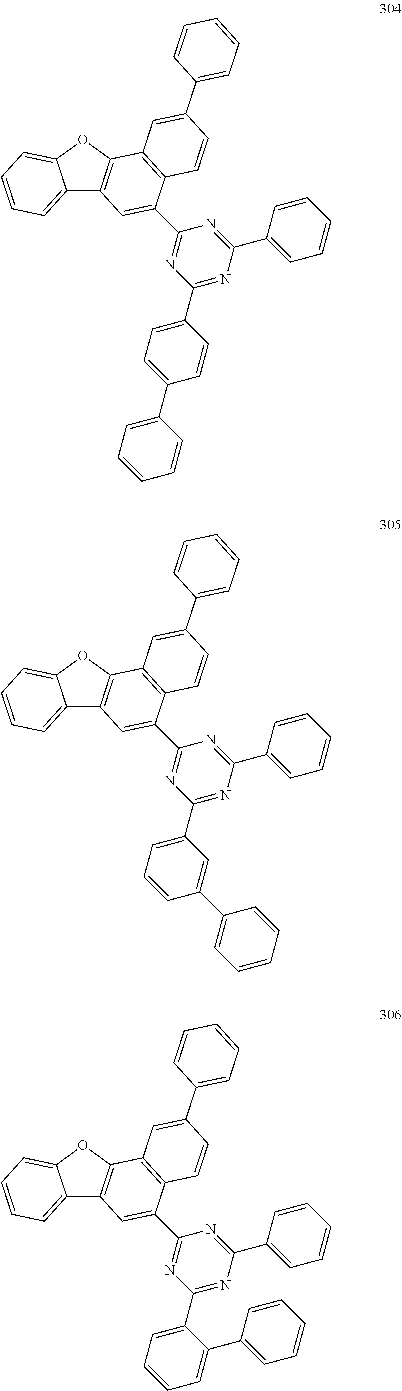 [SMILES: c1ccc(-c2ccc(-c3nc(-c4ccccc4)nc(-c4cc5c6ccccc6oc5c5cc(-c6ccccc6)ccc45)n3)cc2)cc1 ; c1ccc(-c2cccc(-c3nc(-c4ccccc4)nc(-c4cc5c6ccccc6oc5c5cc(-c6ccccc6)ccc45)n3)c2)cc1 ; c1ccc(-c2ccc3c(-c4nc(-c5ccccc5)nc(-c5ccccc5-c5ccccc5)n4)cc4c5ccccc5oc4c3c2)cc1]

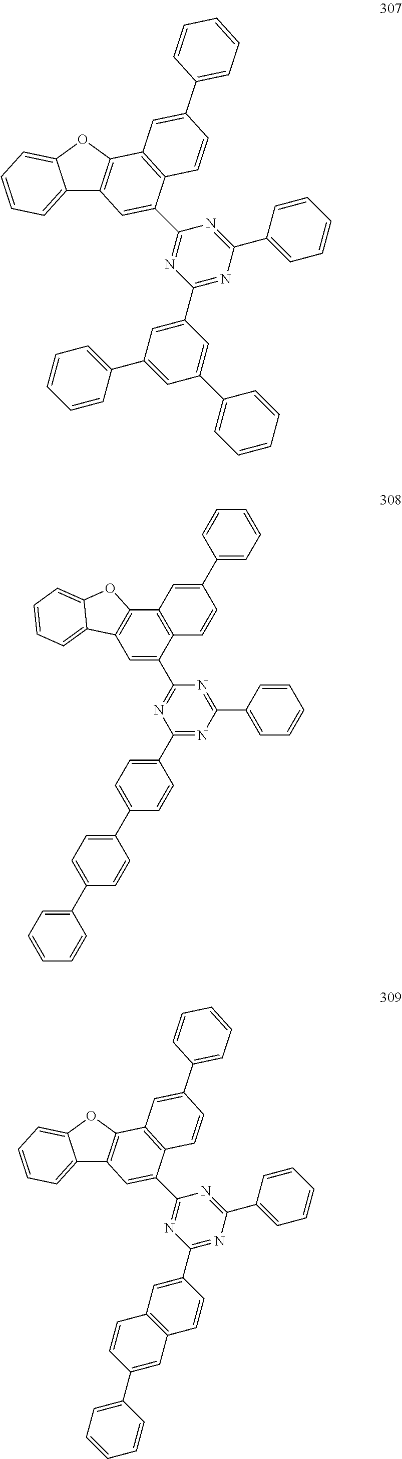 [SMILES: c1ccc(-c2cc(-c3ccccc3)cc(-c3nc(-c4ccccc4)nc(-c4cc5c6ccccc6oc5c5cc(-c6ccccc6)ccc45)n3)c2)cc1 ; c1ccc(-c2ccc(-c3ccc(-c4nc(-c5ccccc5)nc(-c5cc6c7ccccc7oc6c6cc(-c7ccccc7)ccc56)n4)cc3)cc2)cc1 ; c1ccc(-c2ccc3cc(-c4nc(-c5ccccc5)nc(-c5cc6c7ccccc7oc6c6cc(-c7ccccc7)ccc56)n4)ccc3c2)cc1]

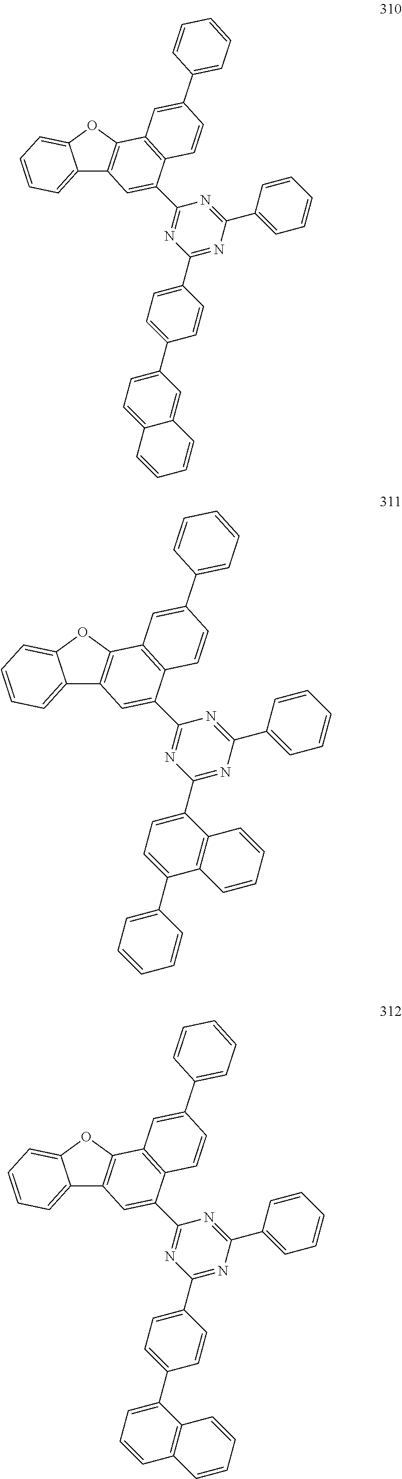 [SMILES: c1ccc(-c2ccc3c(-c4nc(-c5ccccc5)nc(-c5ccc(-c6ccc7ccccc7c6)cc5)n4)cc4c5ccccc5oc4c3c2)cc1 ; c1ccc(-c2ccc3c(-c4nc(-c5ccccc5)nc(-c5ccc(-c6ccccc6)c6ccccc56)n4)cc4c5ccccc5oc4c3c2)cc1 ; c1ccc(-c2ccc3c(-c4nc(-c5ccccc5)nc(-c5ccc(-c6cccc7ccccc67)cc5)n4)cc4c5ccccc5oc4c3c2)cc1]

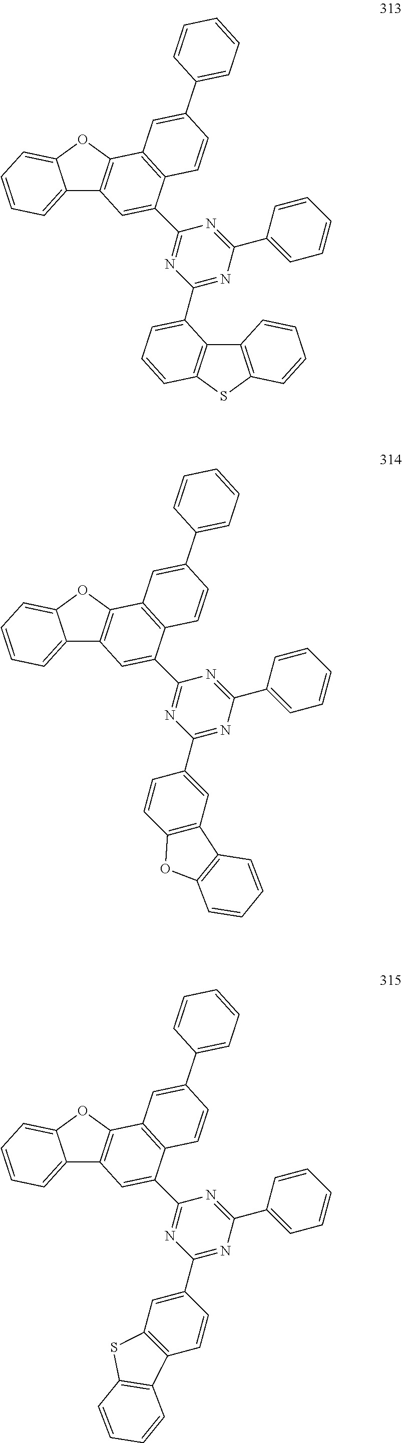 [SMILES: c1ccc(-c2ccc3c(-c4nc(-c5ccccc5)nc(-c5cccc6sc7ccccc7c56)n4)cc4c5ccccc5oc4c3c2)cc1 ; c1ccc(-c2ccc3c(-c4nc(-c5ccccc5)nc(-c5ccc6oc7ccccc7c6c5)n4)cc4c5ccccc5oc4c3c2)cc1 ; c1ccc(-c2ccc3c(-c4nc(-c5ccccc5)nc(-c5ccc6c(c5)sc5ccccc56)n4)cc4c5ccccc5oc4c3c2)cc1]

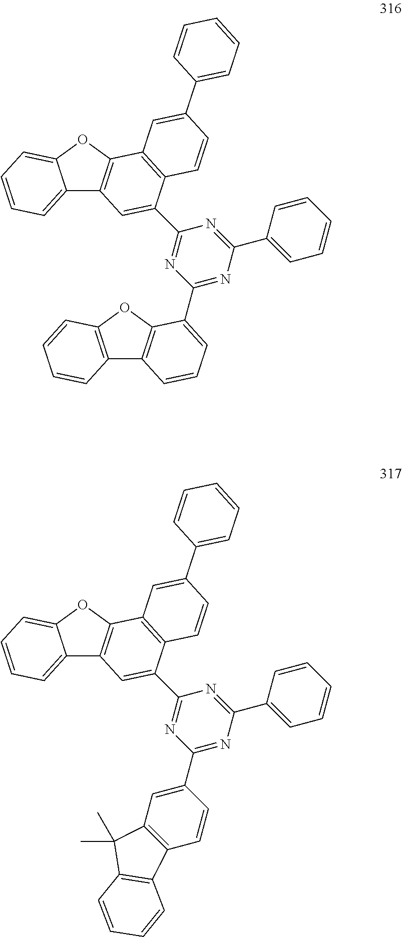 [SMILES: c1ccc(-c2ccc3c(-c4nc(-c5ccccc5)nc(-c5cccc6c5oc5ccccc56)n4)cc4c5ccccc5oc4c3c2)cc1 ; CC1(C)c2ccccc2-c2ccc(-c3nc(-c4ccccc4)nc(-c4cc5c6ccccc6oc5c5cc(-c6ccccc6)ccc45)n3)cc21]

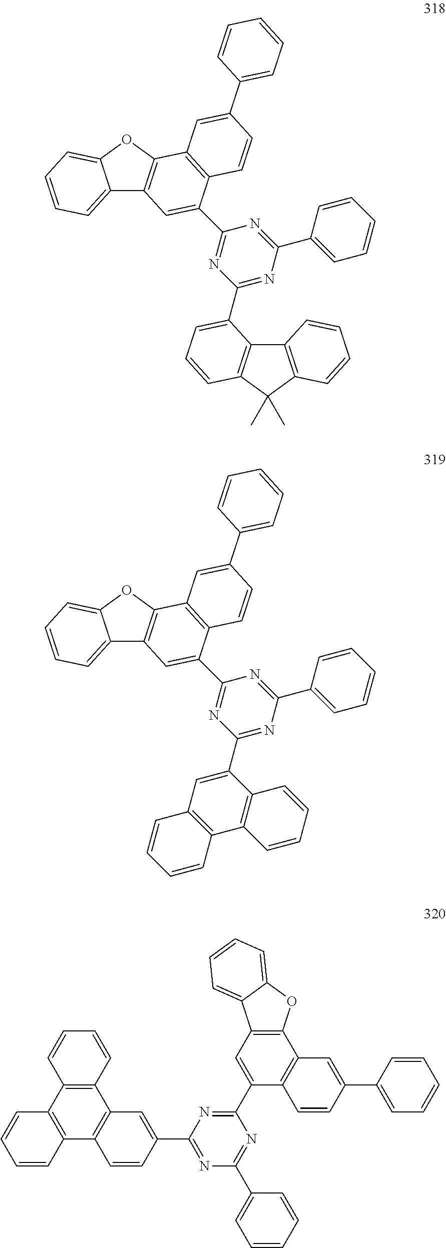

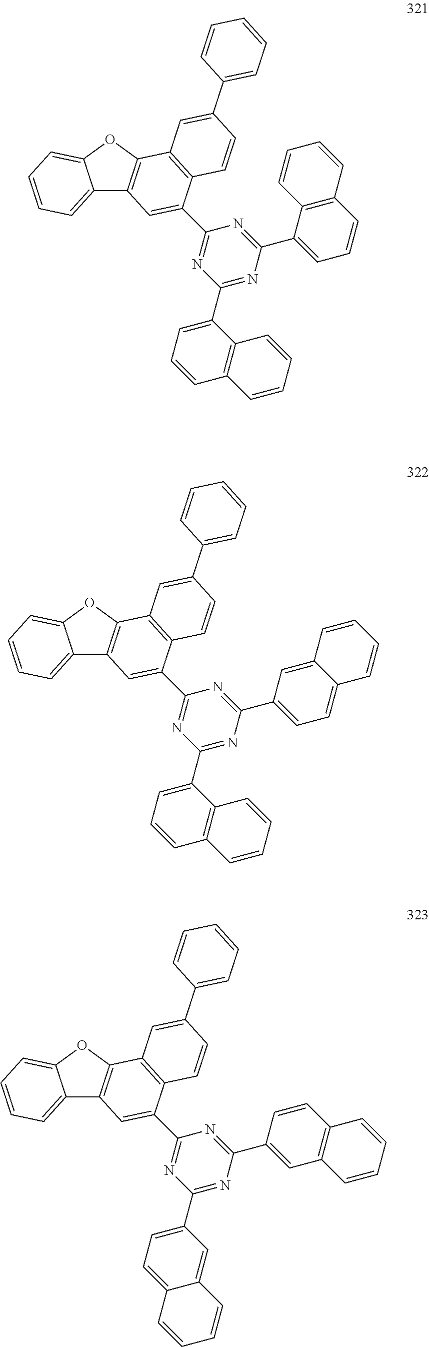 [SMILES: c1ccc(-c2ccc3c(-c4nc(-c5cccc6ccccc56)nc(-c5cccc6ccccc56)n4)cc4c5ccccc5oc4c3c2)cc1 ; c1ccc(-c2ccc3c(-c4nc(-c5ccc6ccccc6c5)nc(-c5cccc6ccccc56)n4)cc4c5ccccc5oc4c3c2)cc1 ; c1ccc(-c2ccc3c(-c4nc(-c5ccc6ccccc6c5)nc(-c5ccc6ccccc6c5)n4)cc4c5ccccc5oc4c3c2)cc1]

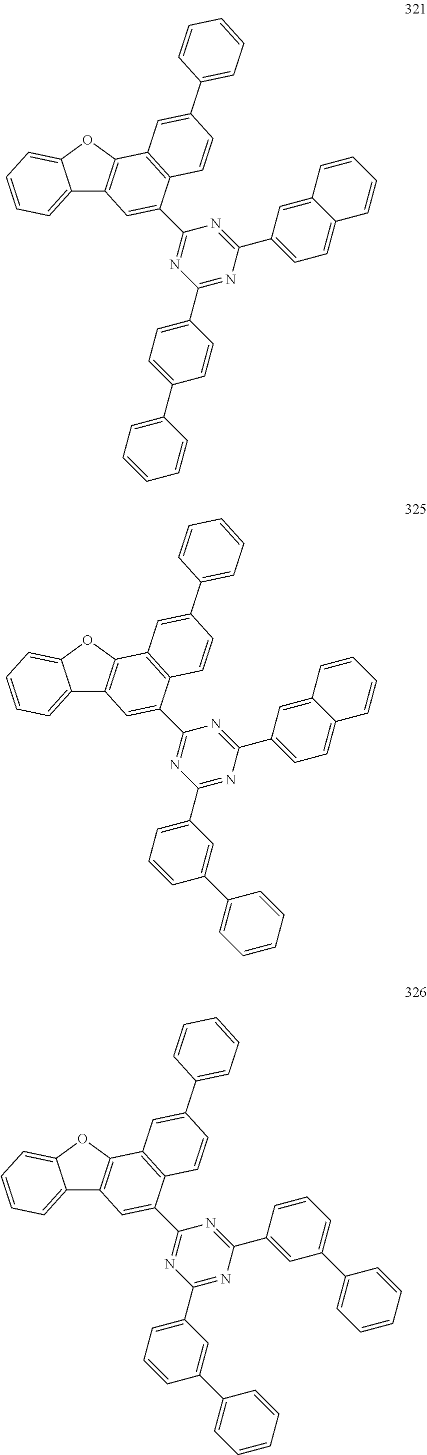 [SMILES: c1ccc(-c2ccc(-c3nc(-c4ccc5ccccc5c4)nc(-c4cc5c6ccccc6oc5c5cc(-c6ccccc6)ccc45)n3)cc2)cc1 ; c1ccc(-c2cccc(-c3nc(-c4ccc5ccccc5c4)nc(-c4cc5c6ccccc6oc5c5cc(-c6ccccc6)ccc45)n3)c2)cc1 ; c1ccc(-c2cccc(-c3nc(-c4cccc(-c5ccccc5)c4)nc(-c4cc5c6ccccc6oc5c5cc(-c6ccccc6)ccc45)n3)c2)cc1]

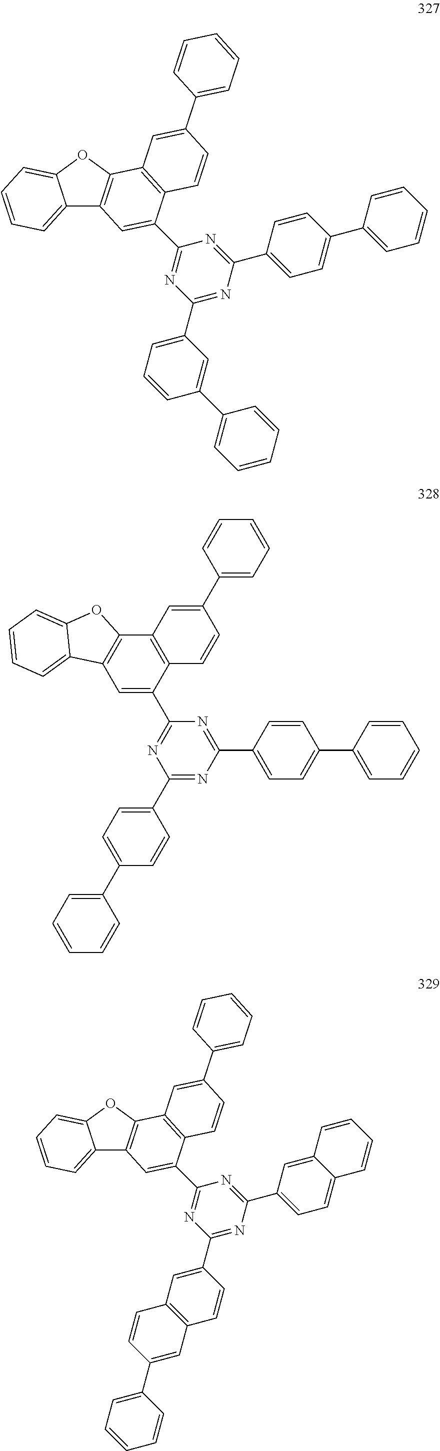 [SMILES: c1ccc(-c2ccc(-c3nc(-c4cccc(-c5ccccc5)c4)nc(-c4cc5c6ccccc6oc5c5cc(-c6ccccc6)ccc45)n3)cc2)cc1 ; c1ccc(-c2ccc(-c3nc(-c4ccc(-c5ccccc5)cc4)nc(-c4cc5c6ccccc6oc5c5cc(-c6ccccc6)ccc45)n3)cc2)cc1 ; c1ccc(-c2ccc3cc(-c4nc(-c5ccc6ccccc6c5)nc(-c5cc6c7ccccc7oc6c6cc(-c7ccccc7)ccc56)n4)ccc3c2)cc1]

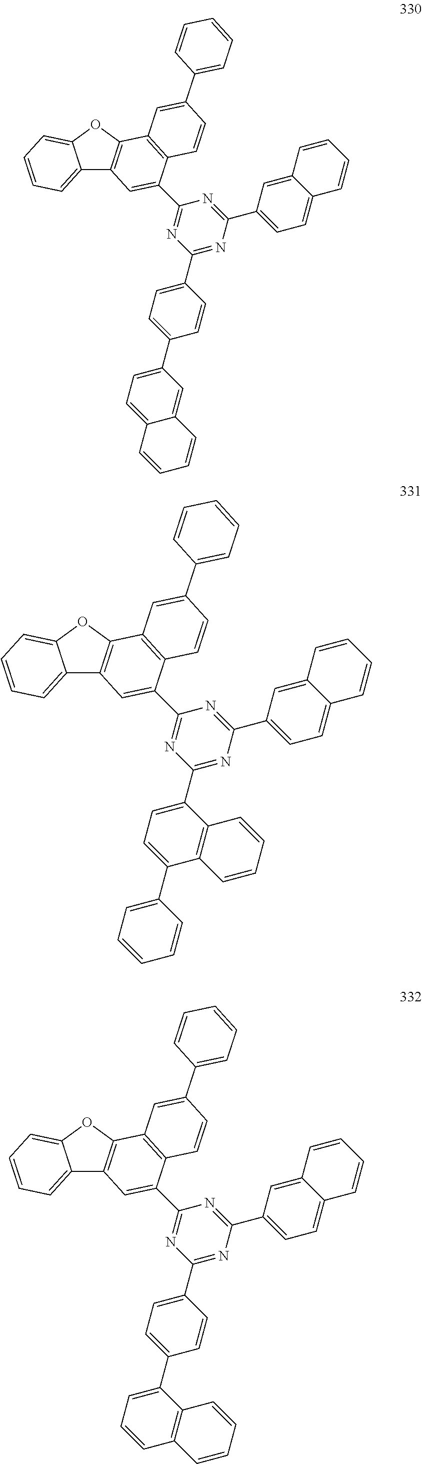

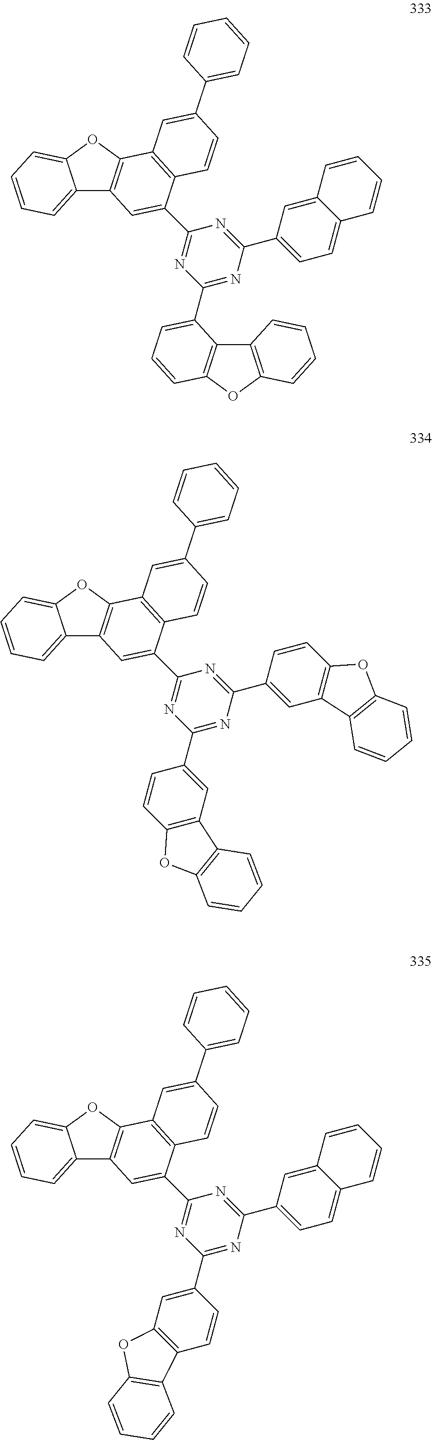 [SMILES: c1ccc(-c2ccc3c(-c4nc(-c5ccc6ccccc6c5)nc(-c5cccc6oc7ccccc7c56)n4)cc4c5ccccc5oc4c3c2)cc1 ; c1ccc(-c2ccc3c(-c4nc(-c5ccc6oc7ccccc7c6c5)nc(-c5ccc6oc7ccccc7c6c5)n4)cc4c5ccccc5oc4c3c2)cc1 ; c1ccc(-c2ccc3c(-c4nc(-c5ccc6ccccc6c5)nc(-c5ccc6c(c5)oc5ccccc56)n4)cc4c5ccccc5oc4c3c2)cc1]

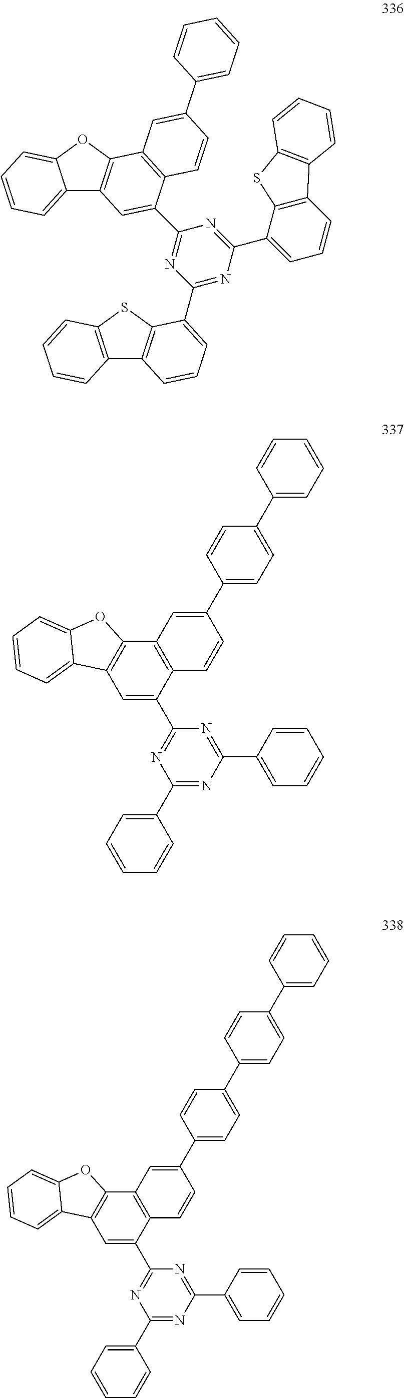 [SMILES: c1ccc(-c2ccc3c(-c4nc(-c5cccc6c5sc5ccccc56)nc(-c5cccc6c5sc5ccccc56)n4)cc4c5ccccc5oc4c3c2)cc1 ; c1ccc(-c2ccc(-c3ccc4c(-c5nc(-c6ccccc6)nc(-c6ccccc6)n5)cc5c6ccccc6oc5c4c3)cc2)cc1 ; c1ccc(-c2ccc(-c3ccc(-c4ccc5c(-c6nc(-c7ccccc7)nc(-c7ccccc7)n6)cc6c7ccccc7oc6c5c4)cc3)cc2)cc1]

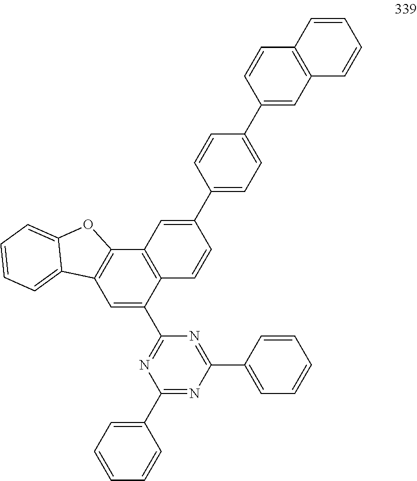 [SMILES: c1ccc(-c2nc(-c3ccccc3)nc(-c3cc4c5ccccc5oc4c4cc(-c5ccc(-c6ccc7ccccc7c6)cc5)ccc34)n2)cc1]

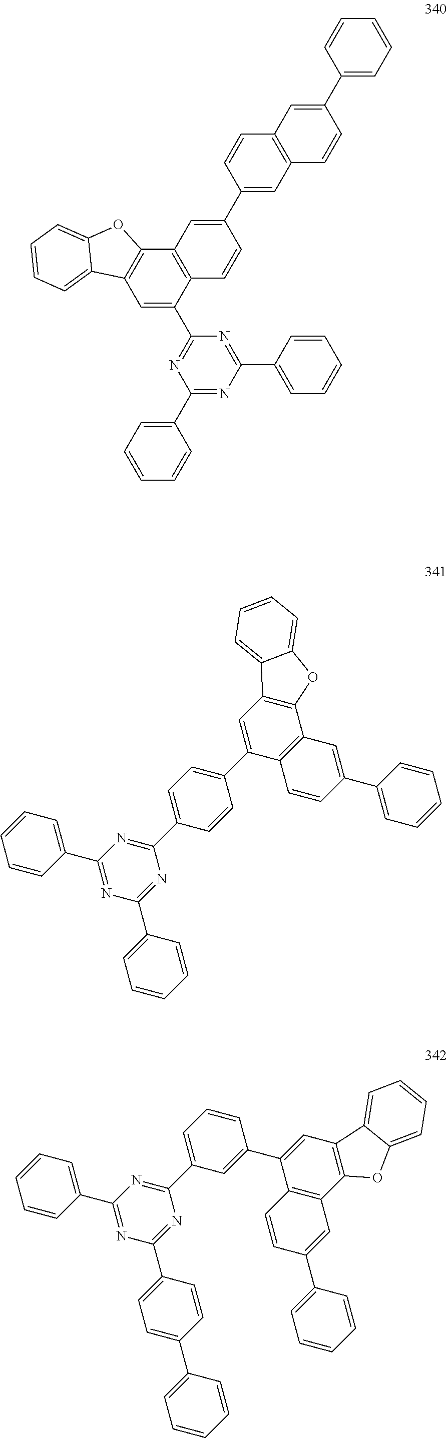 [SMILES: c1ccc(-c2ccc3cc(-c4ccc5c(-c6nc(-c7ccccc7)nc(-c7ccccc7)n6)cc6c7ccccc7oc6c5c4)ccc3c2)cc1 ; c1ccc(-c2ccc3c(-c4ccc(-c5nc(-c6ccccc6)nc(-c6ccccc6)n5)cc4)cc4c5ccccc5oc4c3c2)cc1 ; c1ccc(-c2ccc(-c3nc(-c4ccccc4)nc(-c4cccc(-c5cc6c7ccccc7oc6c6cc(-c7ccccc7)ccc56)c4)n3)cc2)cc1]

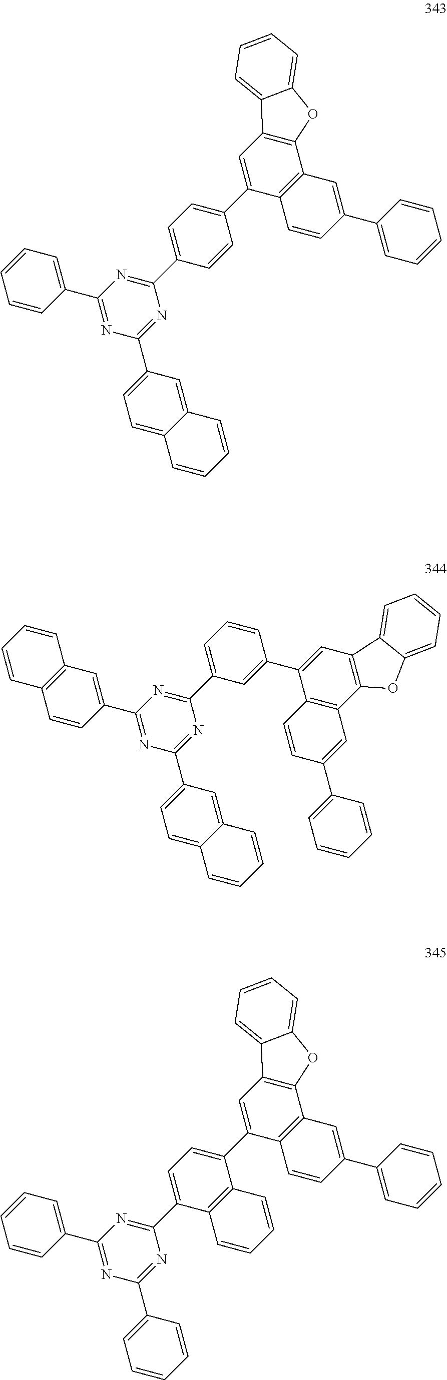 [SMILES: c1ccc(-c2ccc3c(-c4ccc(-c5nc(-c6ccccc6)nc(-c6ccc7ccccc7c6)n5)cc4)cc4c5ccccc5oc4c3c2)cc1 ; c1ccc(-c2ccc3c(-c4cccc(-c5nc(-c6ccc7ccccc7c6)nc(-c6ccc7ccccc7c6)n5)c4)cc4c5ccccc5oc4c3c2)cc1 ; c1ccc(-c2ccc3c(-c4ccc(-c5nc(-c6ccccc6)nc(-c6ccccc6)n5)c5ccccc45)cc4c5ccccc5oc4c3c2)cc1]

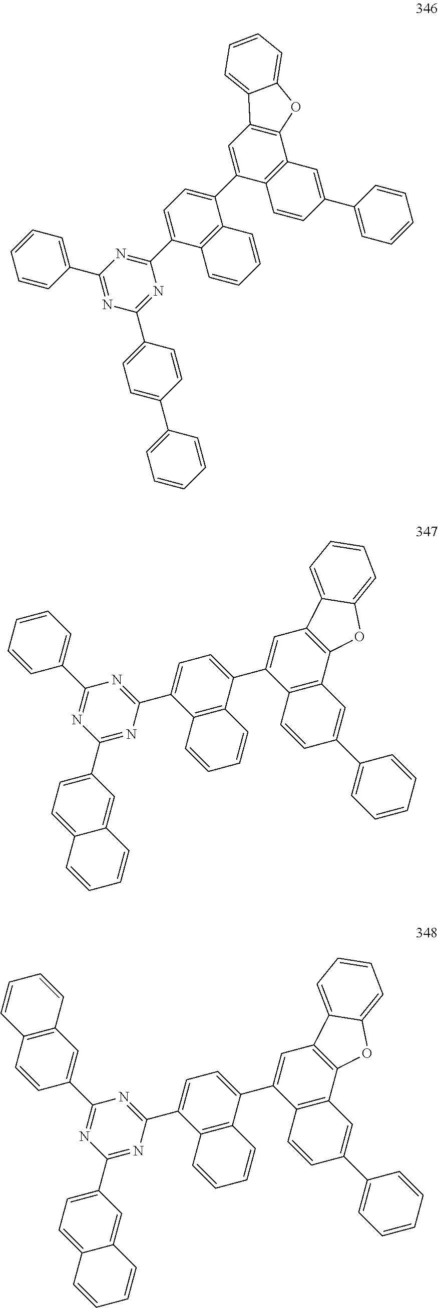 [SMILES: c1ccc(-c2ccc(-c3nc(-c4ccccc4)nc(-c4ccc(-c5cc6c7ccccc7oc6c6cc(-c7ccccc7)ccc56)c5ccccc45)n3)cc2)cc1 ; c1ccc(-c2ccc3c(-c4ccc(-c5nc(-c6ccccc6)nc(-c6ccc7ccccc7c6)n5)c5ccccc45)cc4c5ccccc5oc4c3c2)cc1 ; c1ccc(-c2ccc3c(-c4ccc(-c5nc(-c6ccc7ccccc7c6)nc(-c6ccc7ccccc7c6)n5)c5ccccc45)cc4c5ccccc5oc4c3c2)cc1]

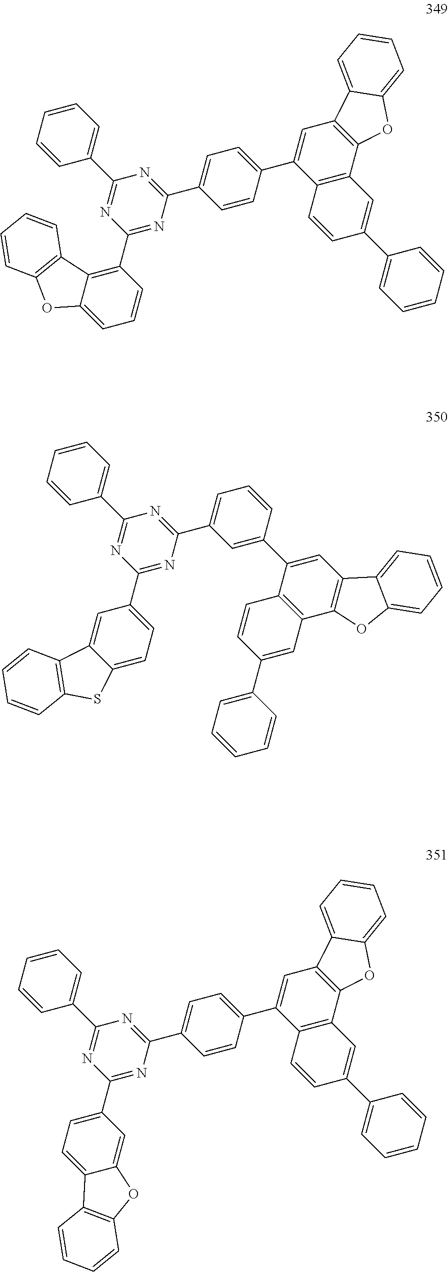 [SMILES: c1ccc(-c2ccc3c(-c4ccc(-c5nc(-c6ccccc6)nc(-c6cccc7oc8ccccc8c67)n5)cc4)cc4c5ccccc5oc4c3c2)cc1 ; c1ccc(-c2ccc3c(-c4cccc(-c5nc(-c6ccccc6)nc(-c6ccc7sc8ccccc8c7c6)n5)c4)cc4c5ccccc5oc4c3c2)cc1 ; c1ccc(-c2ccc3c(-c4ccc(-c5nc(-c6ccccc6)nc(-c6ccc7c(c6)oc6ccccc67)n5)cc4)cc4c5ccccc5oc4c3c2)cc1]

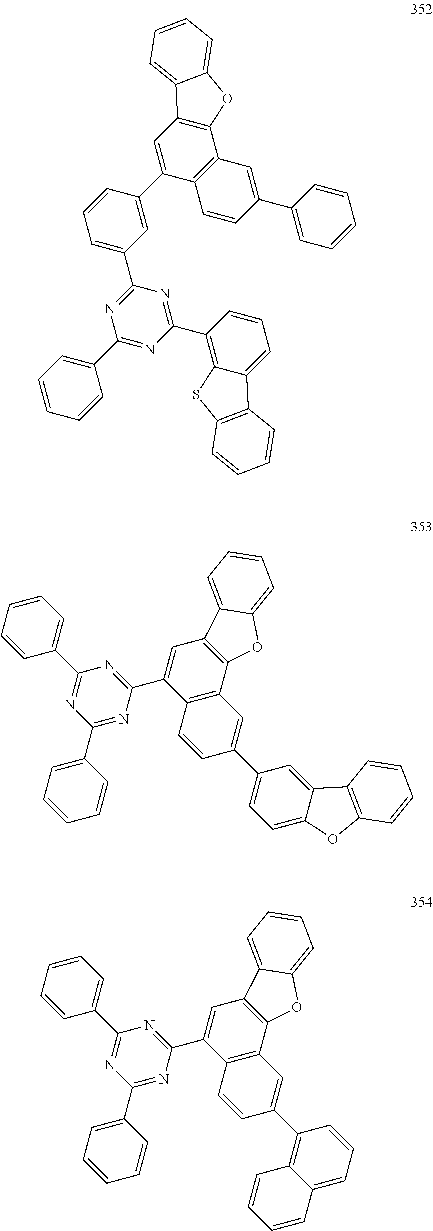 [SMILES: c1ccc(-c2ccc3c(-c4cccc(-c5nc(-c6ccccc6)nc(-c6cccc7c6sc6ccccc67)n5)c4)cc4c5ccccc5oc4c3c2)cc1 ; c1ccc(-c2nc(-c3ccccc3)nc(-c3cc4c5ccccc5oc4c4cc(-c5ccc6oc7ccccc7c6c5)ccc34)n2)cc1 ; c1ccc(-c2nc(-c3ccccc3)nc(-c3cc4c5ccccc5oc4c4cc(-c5cccc6ccccc56)ccc34)n2)cc1]

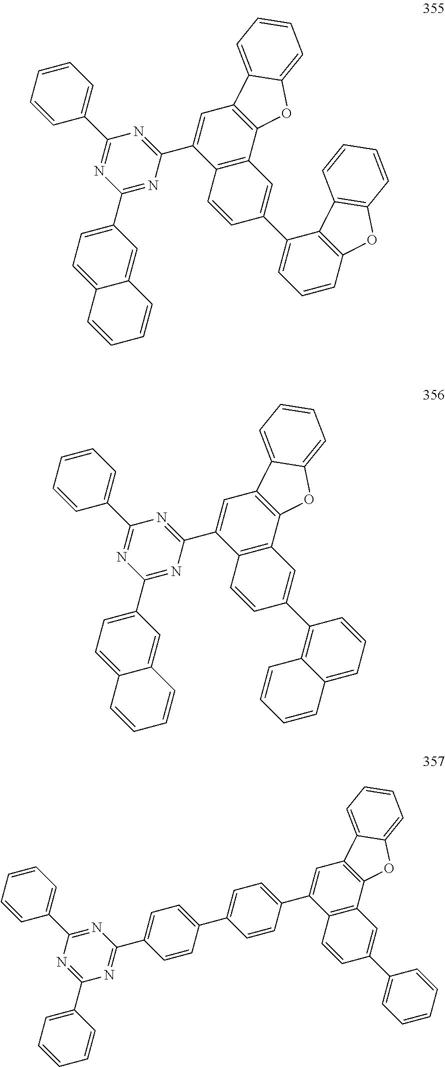 [SMILES: c1ccc(-c2nc(-c3ccc4ccccc4c3)nc(-c3cc4c5ccccc5oc4c4cc(-c5cccc6oc7ccccc7c56)ccc34)n2)cc1 ; c1ccc(-c2nc(-c3ccc4ccccc4c3)nc(-c3cc4c5ccccc5oc4c4cc(-c5cccc6ccccc56)ccc34)n2)cc1 ; c1ccc(-c2ccc3c(-c4ccc(-c5ccc(-c6nc(-c7ccccc7)nc(-c7ccccc7)n6)cc5)cc4)cc4c5ccccc5oc4c3c2)cc1]

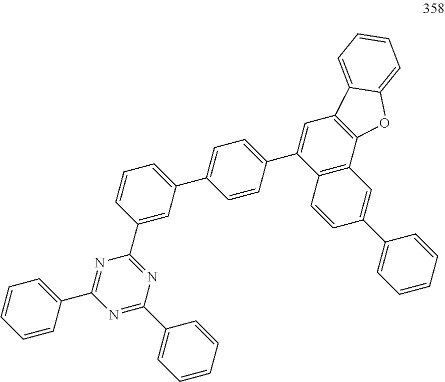 [SMILES: c1ccc(-c2ccc3c(-c4ccc(-c5cccc(-c6nc(-c7ccccc7)nc(-c7ccccc7)n6)c5)cc4)cc4c5ccccc5oc4c3c2)cc1]

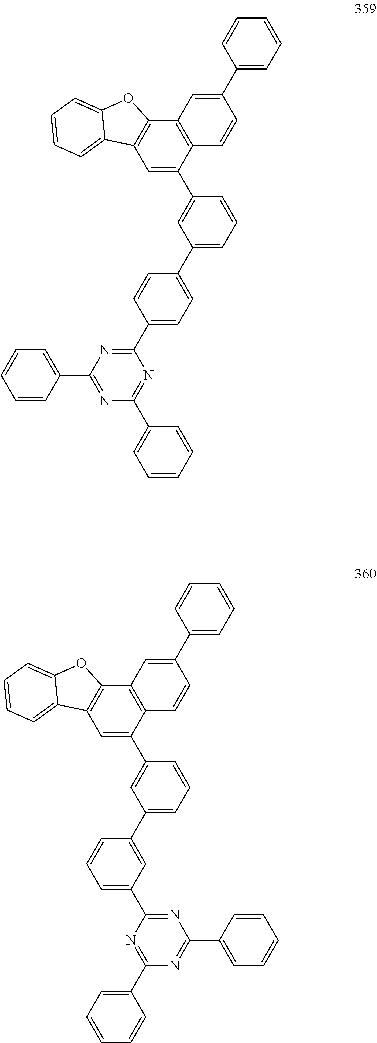 [SMILES: c1ccc(-c2ccc3c(-c4cccc(-c5ccc(-c6nc(-c7ccccc7)nc(-c7ccccc7)n6)cc5)c4)cc4c5ccccc5oc4c3c2)cc1 ; c1ccc(-c2ccc3c(-c4cccc(-c5cccc(-c6nc(-c7ccccc7)nc(-c7ccccc7)n6)c5)c4)cc4c5ccccc5oc4c3c2)cc1]

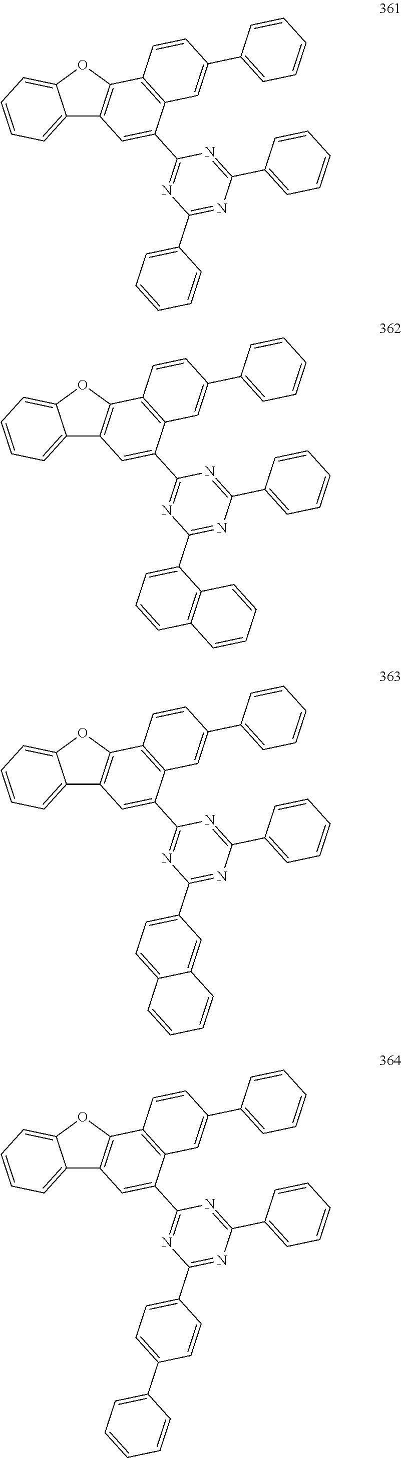 [SMILES: c1ccc(-c2ccc3c(c2)c(-c2nc(-c4ccccc4)nc(-c4ccccc4)n2)cc2c4ccccc4oc32)cc1 ; c1ccc(-c2ccc3c(c2)c(-c2nc(-c4ccccc4)nc(-c4cccc5ccccc45)n2)cc2c4ccccc4oc32)cc1 ; c1ccc(-c2ccc3c(c2)c(-c2nc(-c4ccccc4)nc(-c4ccc5ccccc5c4)n2)cc2c4ccccc4oc32)cc1 ; c1ccc(-c2ccc(-c3nc(-c4ccccc4)nc(-c4cc5c6ccccc6oc5c5ccc(-c6ccccc6)cc45)n3)cc2)cc1]

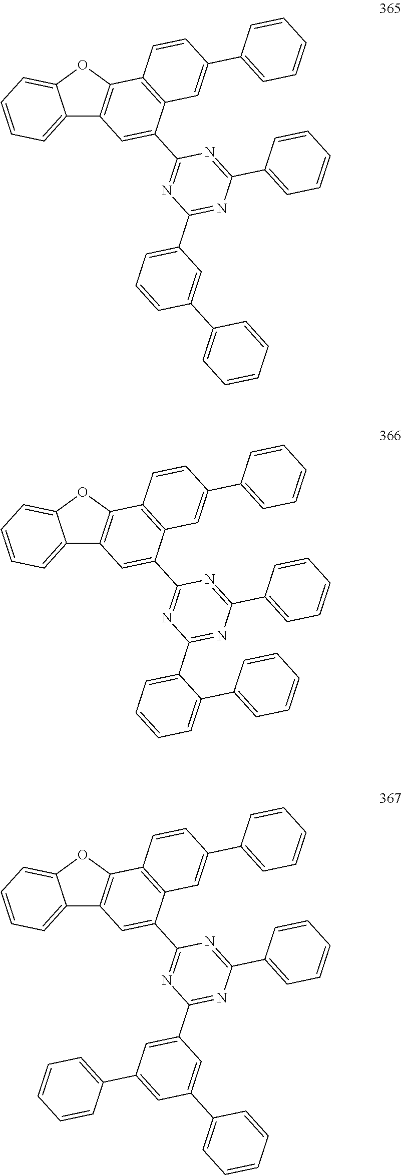 [SMILES: c1ccc(-c2cccc(-c3nc(-c4ccccc4)nc(-c4cc5c6ccccc6oc5c5ccc(-c6ccccc6)cc45)n3)c2)cc1 ; c1ccc(-c2ccc3c(c2)c(-c2nc(-c4ccccc4)nc(-c4ccccc4-c4ccccc4)n2)cc2c4ccccc4oc32)cc1 ; c1ccc(-c2cc(-c3ccccc3)cc(-c3nc(-c4ccccc4)nc(-c4cc5c6ccccc6oc5c5ccc(-c6ccccc6)cc45)n3)c2)cc1]

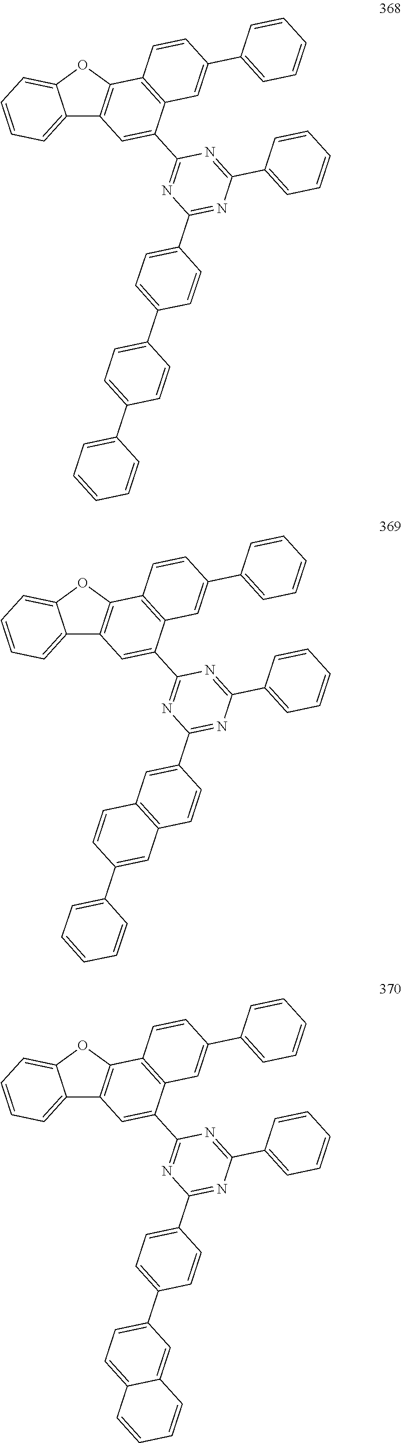 [SMILES: c1ccc(-c2ccc(-c3ccc(-c4nc(-c5ccccc5)nc(-c5cc6c7ccccc7oc6c6ccc(-c7ccccc7)cc56)n4)cc3)cc2)cc1 ; c1ccc(-c2ccc3cc(-c4nc(-c5ccccc5)nc(-c5cc6c7ccccc7oc6c6ccc(-c7ccccc7)cc56)n4)ccc3c2)cc1 ; c1ccc(-c2ccc3c(c2)c(-c2nc(-c4ccccc4)nc(-c4ccc(-c5ccc6ccccc6c5)cc4)n2)cc2c4ccccc4oc32)cc1]

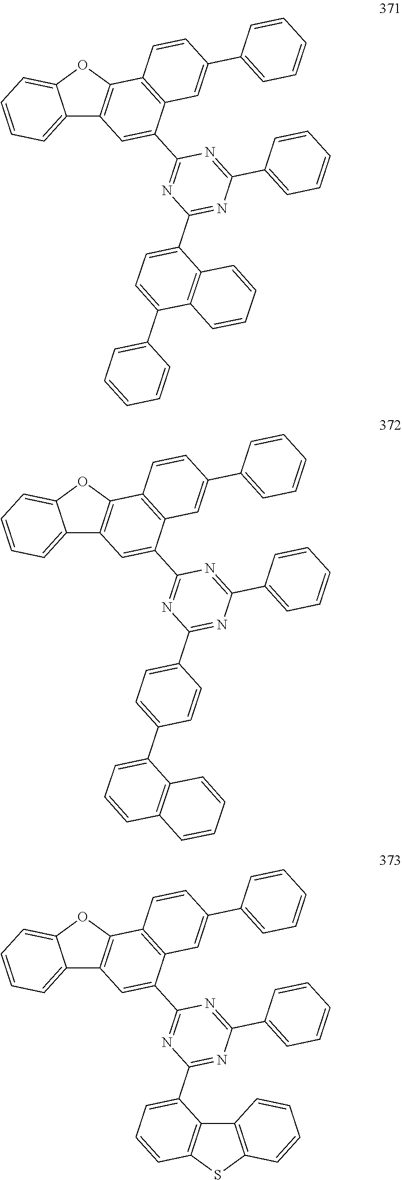 [SMILES: c1ccc(-c2ccc3c(c2)c(-c2nc(-c4ccccc4)nc(-c4ccc(-c5ccccc5)c5ccccc45)n2)cc2c4ccccc4oc32)cc1 ; c1ccc(-c2ccc3c(c2)c(-c2nc(-c4ccccc4)nc(-c4ccc(-c5cccc6ccccc56)cc4)n2)cc2c4ccccc4oc32)cc1 ; c1ccc(-c2ccc3c(c2)c(-c2nc(-c4ccccc4)nc(-c4cccc5sc6ccccc6c45)n2)cc2c4ccccc4oc32)cc1]

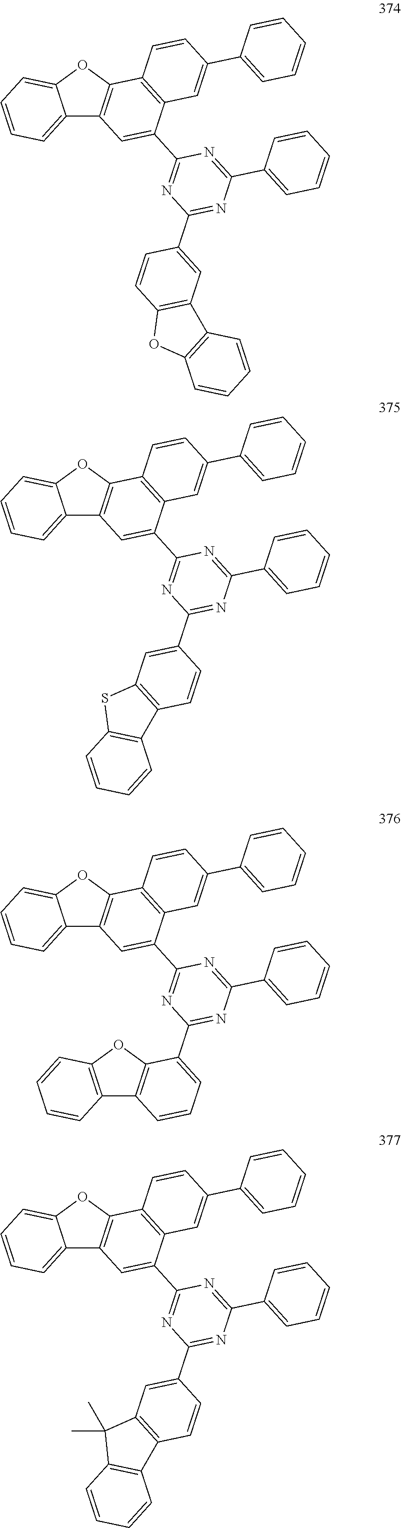

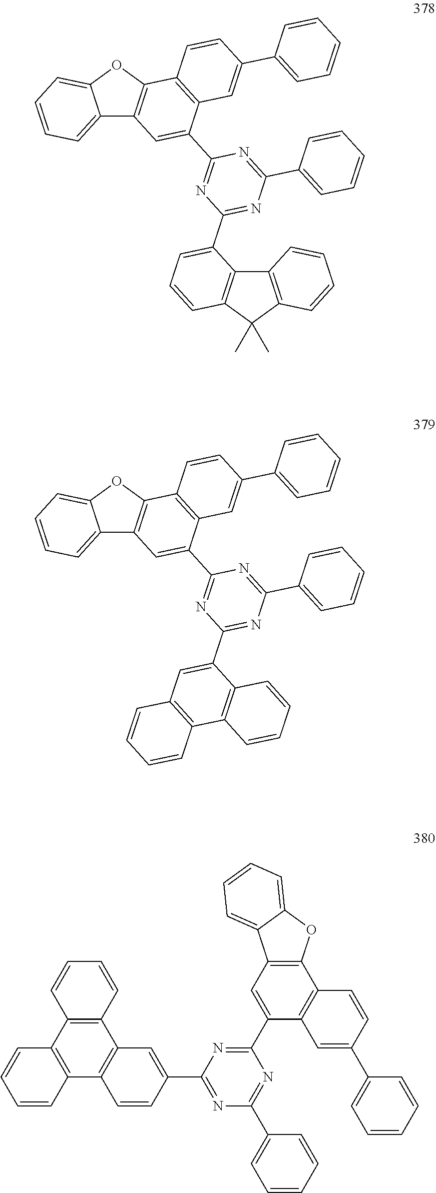 [SMILES: CC1(C)c2ccccc2-c2c(-c3nc(-c4ccccc4)nc(-c4cc5c6ccccc6oc5c5ccc(-c6ccccc6)cc45)n3)cccc21 ; c1ccc(-c2ccc3c(c2)c(-c2nc(-c4ccccc4)nc(-c4cc5ccccc5c5ccccc45)n2)cc2c4ccccc4oc32)cc1 ; c1ccc(-c2ccc3c(c2)c(-c2nc(-c4ccccc4)nc(-c4ccc5c6ccccc6c6ccccc6c5c4)n2)cc2c4ccccc4oc32)cc1]

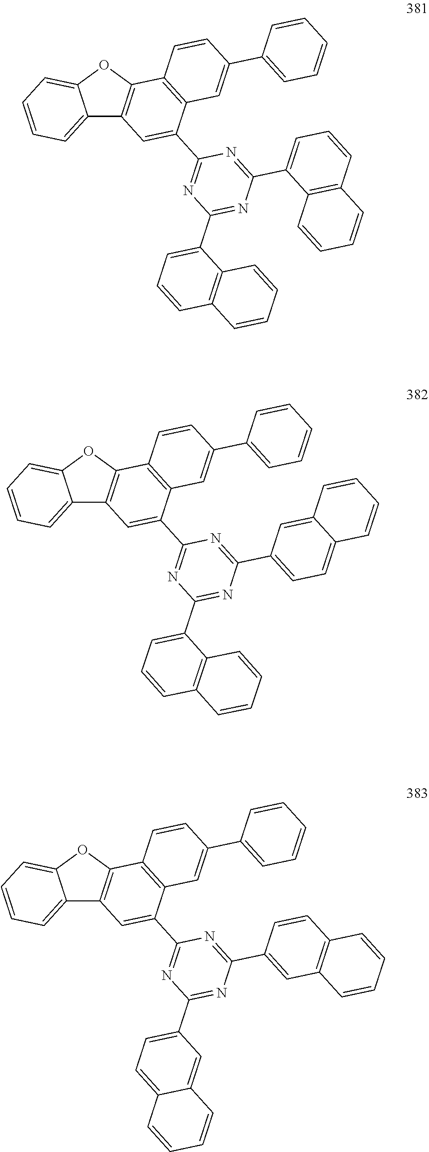 [SMILES: c1ccc(-c2ccc3c(c2)c(-c2nc(-c4cccc5ccccc45)nc(-c4cccc5ccccc45)n2)cc2c4ccccc4oc32)cc1 ; c1ccc(-c2ccc3c(c2)c(-c2nc(-c4ccc5ccccc5c4)nc(-c4cccc5ccccc45)n2)cc2c4ccccc4oc32)cc1 ; c1ccc(-c2ccc3c(c2)c(-c2nc(-c4ccc5ccccc5c4)nc(-c4ccc5ccccc5c4)n2)cc2c4ccccc4oc32)cc1]

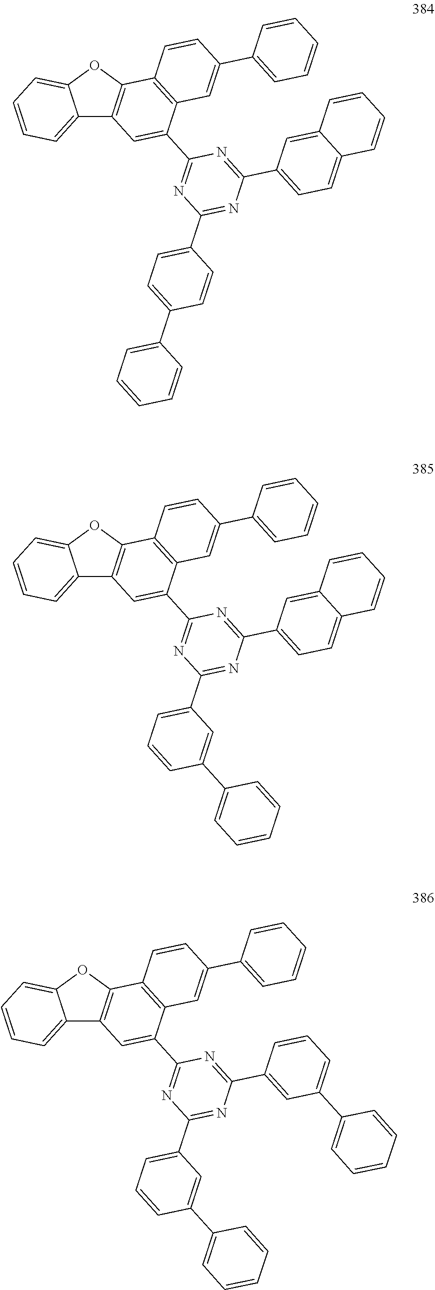 [SMILES: c1ccc(-c2ccc(-c3nc(-c4ccc5ccccc5c4)nc(-c4cc5c6ccccc6oc5c5ccc(-c6ccccc6)cc45)n3)cc2)cc1 ; c1ccc(-c2cccc(-c3nc(-c4ccc5ccccc5c4)nc(-c4cc5c6ccccc6oc5c5ccc(-c6ccccc6)cc45)n3)c2)cc1 ; c1ccc(-c2cccc(-c3nc(-c4cccc(-c5ccccc5)c4)nc(-c4cc5c6ccccc6oc5c5ccc(-c6ccccc6)cc45)n3)c2)cc1]

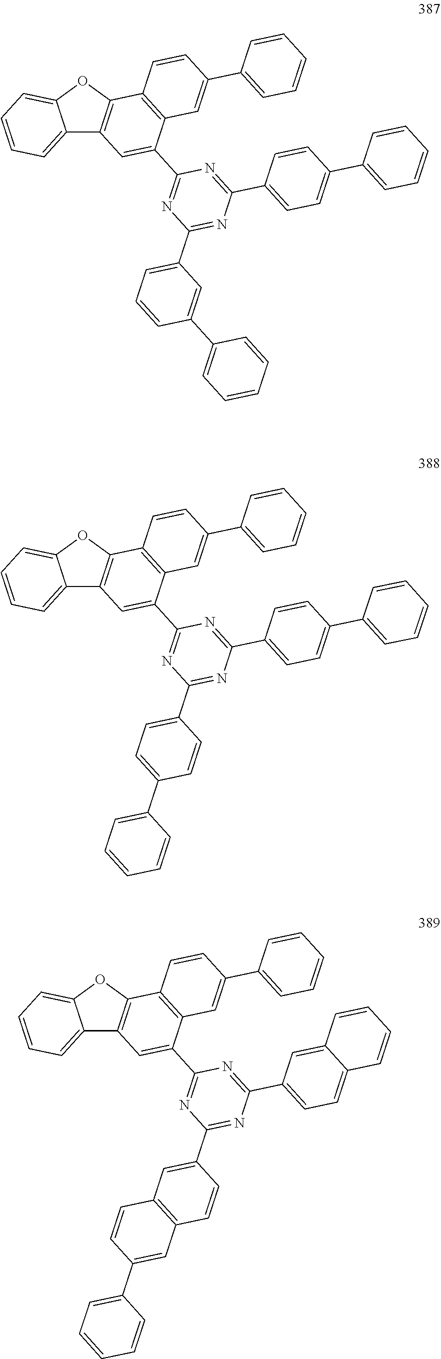 [SMILES: c1ccc(-c2ccc(-c3nc(-c4cccc(-c5ccccc5)c4)nc(-c4cc5c6ccccc6oc5c5ccc(-c6ccccc6)cc45)n3)cc2)cc1 ; c1ccc(-c2ccc(-c3nc(-c4ccc(-c5ccccc5)cc4)nc(-c4cc5c6ccccc6oc5c5ccc(-c6ccccc6)cc45)n3)cc2)cc1 ; c1ccc(-c2ccc3cc(-c4nc(-c5ccc6ccccc6c5)nc(-c5cc6c7ccccc7oc6c6ccc(-c7ccccc7)cc56)n4)ccc3c2)cc1]

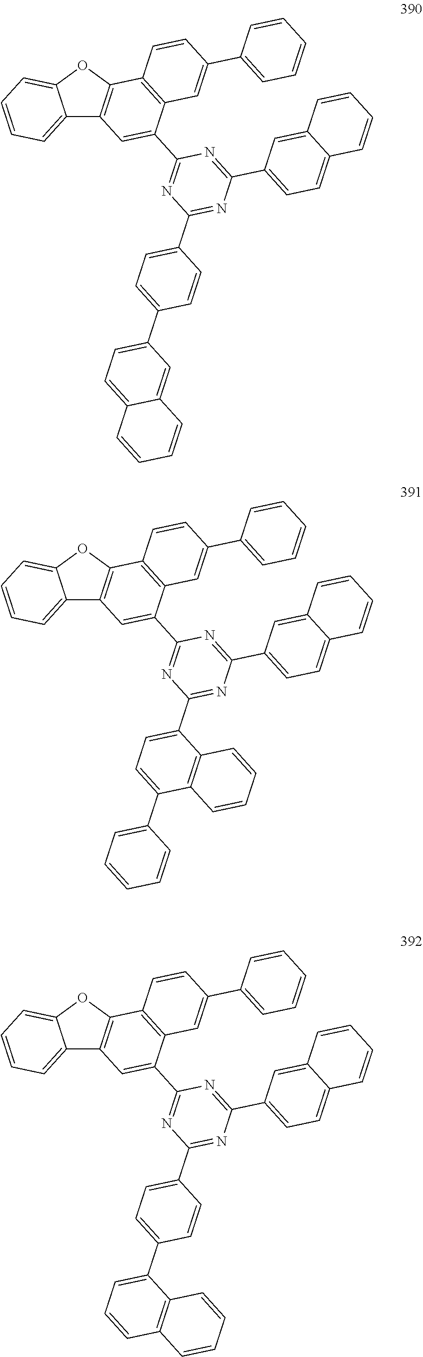 [SMILES: c1ccc(-c2ccc3c(c2)c(-c2nc(-c4ccc(-c5ccc6ccccc6c5)cc4)nc(-c4ccc5ccccc5c4)n2)cc2c4ccccc4oc32)cc1 ; c1ccc(-c2ccc3c(c2)c(-c2nc(-c4ccc5ccccc5c4)nc(-c4ccc(-c5ccccc5)c5ccccc45)n2)cc2c4ccccc4oc32)cc1 ; c1ccc(-c2ccc3c(c2)c(-c2nc(-c4ccc(-c5cccc6ccccc56)cc4)nc(-c4ccc5ccccc5c4)n2)cc2c4ccccc4oc32)cc1]

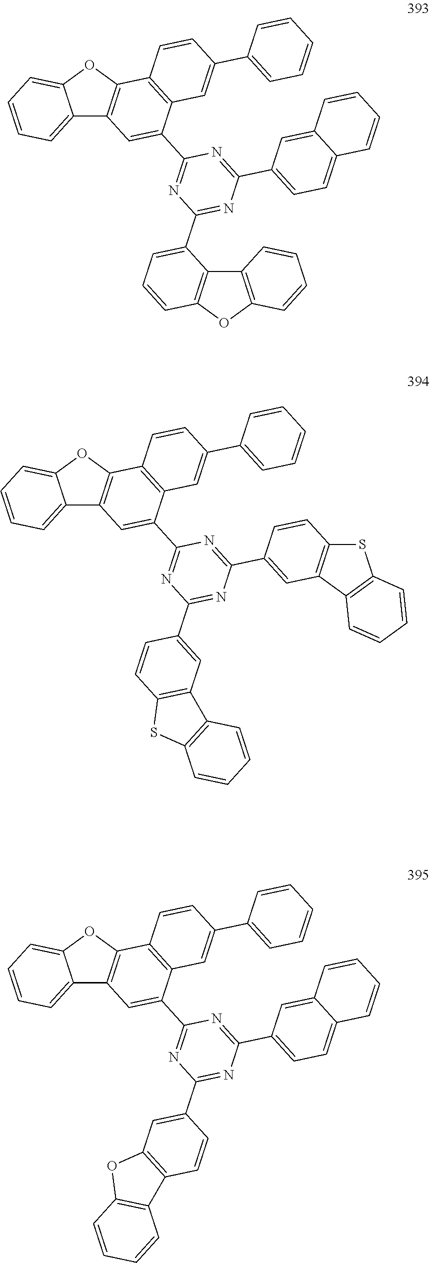 [SMILES: c1ccc(-c2ccc3c(c2)c(-c2nc(-c4ccc5ccccc5c4)nc(-c4cccc5oc6ccccc6c45)n2)cc2c4ccccc4oc32)cc1 ; c1ccc(-c2ccc3c(c2)c(-c2nc(-c4ccc5sc6ccccc6c5c4)nc(-c4ccc5sc6ccccc6c5c4)n2)cc2c4ccccc4oc32)cc1 ; c1ccc(-c2ccc3c(c2)c(-c2nc(-c4ccc5ccccc5c4)nc(-c4ccc5c(c4)oc4ccccc45)n2)cc2c4ccccc4oc32)cc1]

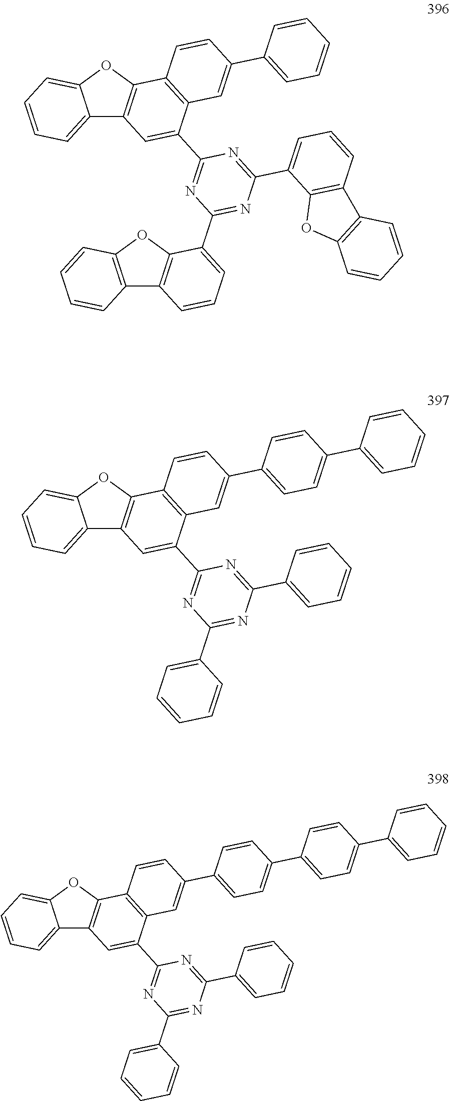 [SMILES: c1ccc(-c2ccc3c(c2)c(-c2nc(-c4cccc5c4oc4ccccc45)nc(-c4cccc5c4oc4ccccc45)n2)cc2c4ccccc4oc32)cc1 ; c1ccc(-c2ccc(-c3ccc4c(c3)c(-c3nc(-c5ccccc5)nc(-c5ccccc5)n3)cc3c5ccccc5oc43)cc2)cc1 ; c1ccc(-c2ccc(-c3ccc(-c4ccc5c(c4)c(-c4nc(-c6ccccc6)nc(-c6ccccc6)n4)cc4c6ccccc6oc54)cc3)cc2)cc1]

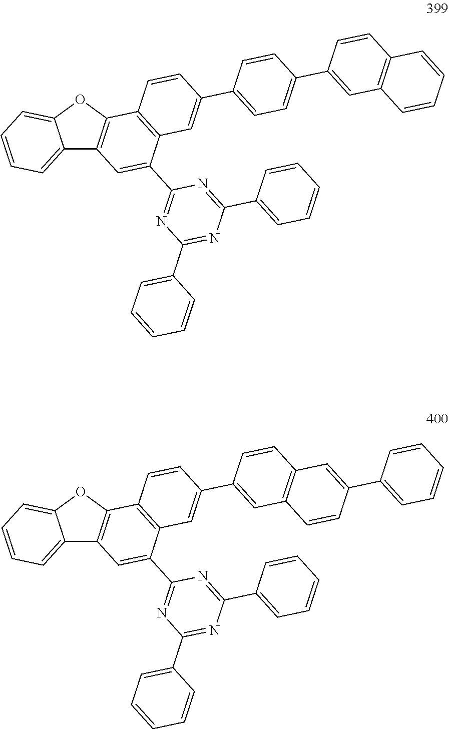 [SMILES: c1ccc(-c2nc(-c3ccccc3)nc(-c3cc4c5ccccc5oc4c4ccc(-c5ccc(-c6ccc7ccccc7c6)cc5)cc34)n2)cc1 ; c1ccc(-c2ccc3cc(-c4ccc5c(c4)c(-c4nc(-c6ccccc6)nc(-c6ccccc6)n4)cc4c6ccccc6oc54)ccc3c2)cc1]

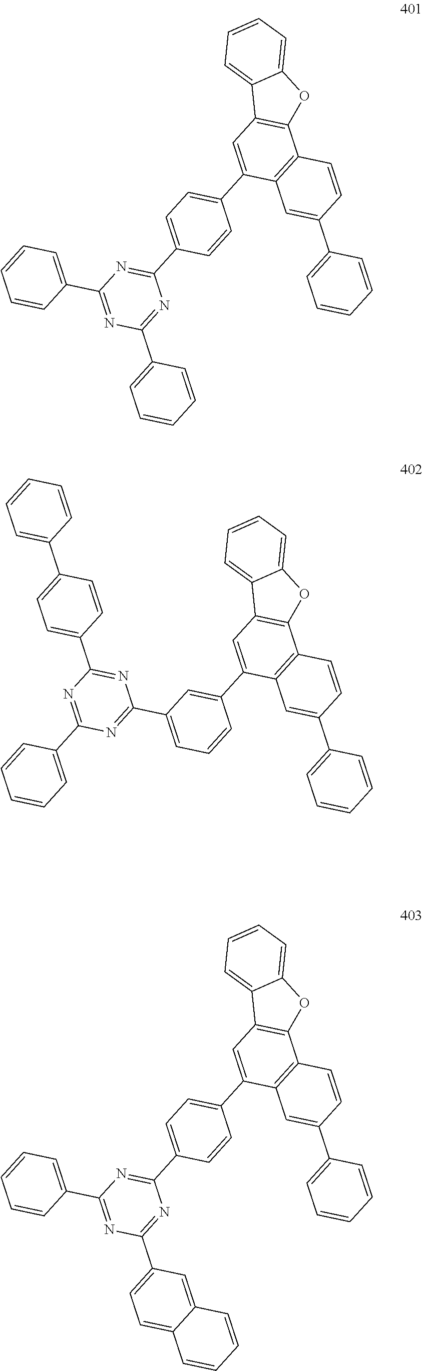 [SMILES: c1ccc(-c2ccc3c(c2)c(-c2ccc(-c4nc(-c5ccccc5)nc(-c5ccccc5)n4)cc2)cc2c4ccccc4oc32)cc1 ; c1ccc(-c2ccc(-c3nc(-c4ccccc4)nc(-c4cccc(-c5cc6c7ccccc7oc6c6ccc(-c7ccccc7)cc56)c4)n3)cc2)cc1 ; c1ccc(-c2ccc3c(c2)c(-c2ccc(-c4nc(-c5ccccc5)nc(-c5ccc6ccccc6c5)n4)cc2)cc2c4ccccc4oc32)cc1]

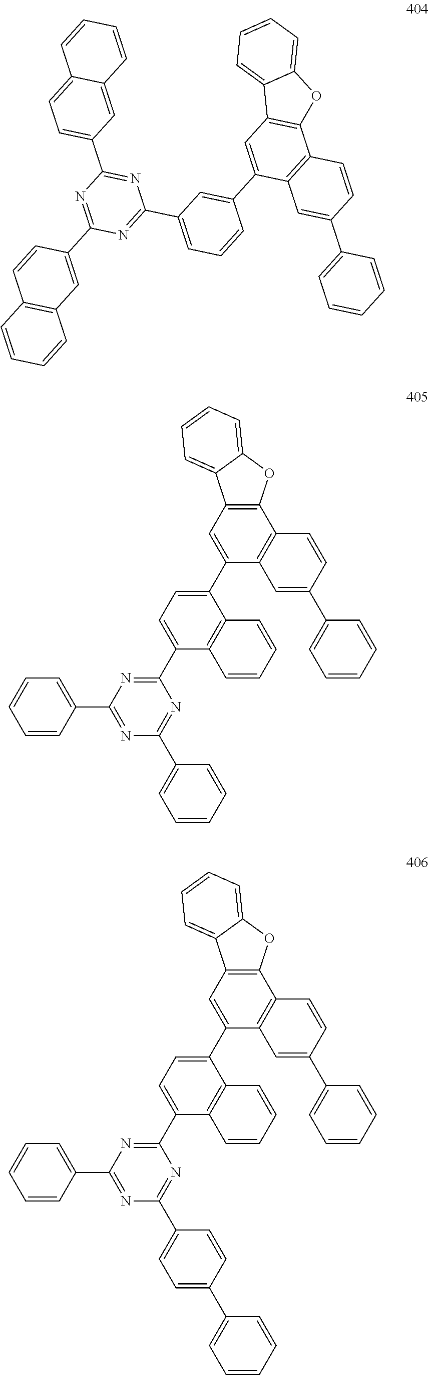 [SMILES: c1ccc(-c2ccc3c(c2)c(-c2cccc(-c4nc(-c5ccc6ccccc6c5)nc(-c5ccc6ccccc6c5)n4)c2)cc2c4ccccc4oc32)cc1 ; c1ccc(-c2ccc3c(c2)c(-c2ccc(-c4nc(-c5ccccc5)nc(-c5ccccc5)n4)c4ccccc24)cc2c4ccccc4oc32)cc1 ; c1ccc(-c2ccc(-c3nc(-c4ccccc4)nc(-c4ccc(-c5cc6c7ccccc7oc6c6ccc(-c7ccccc7)cc56)c5ccccc45)n3)cc2)cc1]

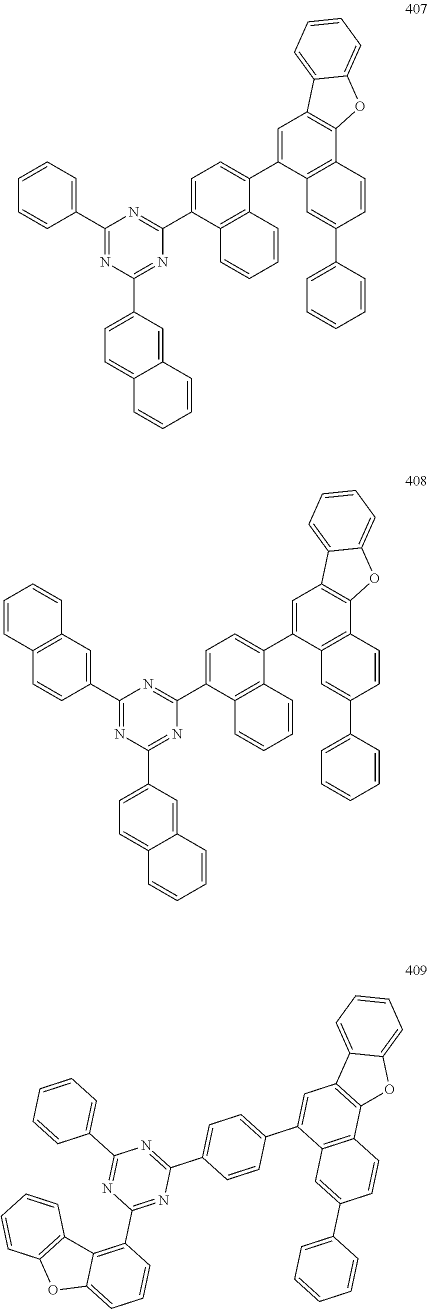 [SMILES: c1ccc(-c2ccc3c(c2)c(-c2ccc(-c4nc(-c5ccccc5)nc(-c5ccc6ccccc6c5)n4)c4ccccc24)cc2c4ccccc4oc32)cc1 ; c1ccc(-c2ccc3c(c2)c(-c2ccc(-c4nc(-c5ccc6ccccc6c5)nc(-c5ccc6ccccc6c5)n4)c4ccccc24)cc2c4ccccc4oc32)cc1 ; c1ccc(-c2ccc3c(c2)c(-c2ccc(-c4nc(-c5ccccc5)nc(-c5cccc6oc7ccccc7c56)n4)cc2)cc2c4ccccc4oc32)cc1]

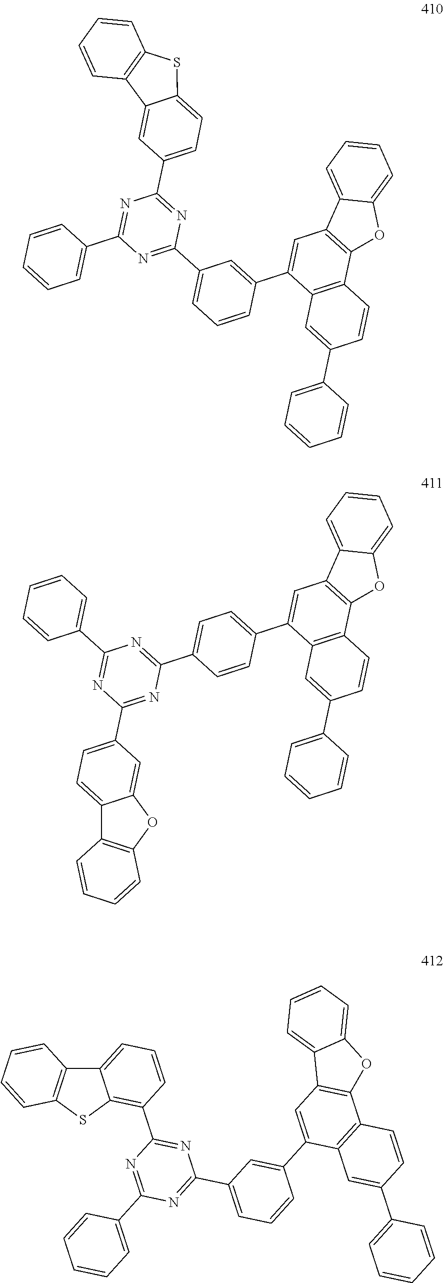 [SMILES: c1ccc(-c2ccc3c(c2)c(-c2cccc(-c4nc(-c5ccccc5)nc(-c5ccc6sc7ccccc7c6c5)n4)c2)cc2c4ccccc4oc32)cc1 ; c1ccc(-c2ccc3c(c2)c(-c2ccc(-c4nc(-c5ccccc5)nc(-c5ccc6c(c5)oc5ccccc56)n4)cc2)cc2c4ccccc4oc32)cc1 ; c1ccc(-c2ccc3c(c2)c(-c2cccc(-c4nc(-c5ccccc5)nc(-c5cccc6c5sc5ccccc56)n4)c2)cc2c4ccccc4oc32)cc1]

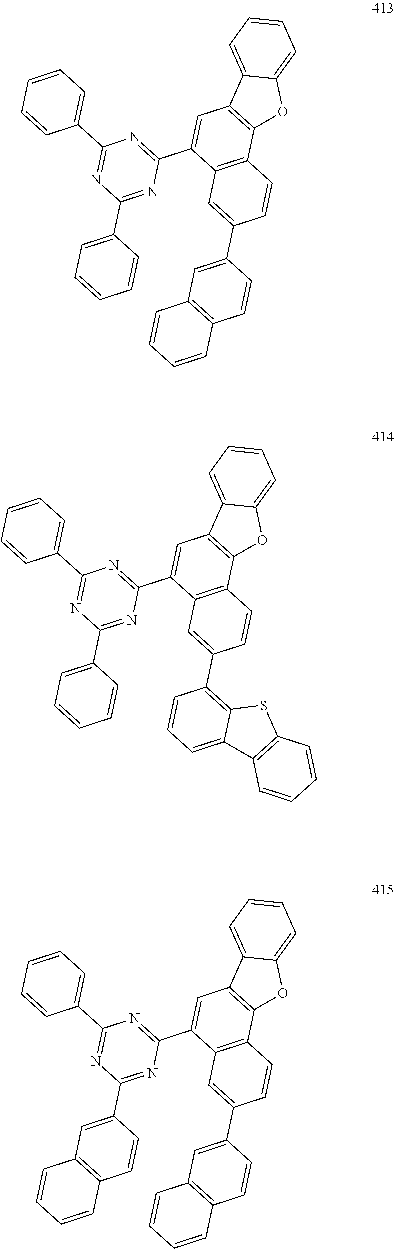 [SMILES: c1ccc(-c2nc(-c3ccccc3)nc(-c3cc4c5ccccc5oc4c4ccc(-c5ccc6ccccc6c5)cc34)n2)cc1 ; c1ccc(-c2nc(-c3ccccc3)nc(-c3cc4c5ccccc5oc4c4ccc(-c5cccc6c5sc5ccccc56)cc34)n2)cc1 ; c1ccc(-c2nc(-c3ccc4ccccc4c3)nc(-c3cc4c5ccccc5oc4c4ccc(-c5ccc6ccccc6c5)cc34)n2)cc1]

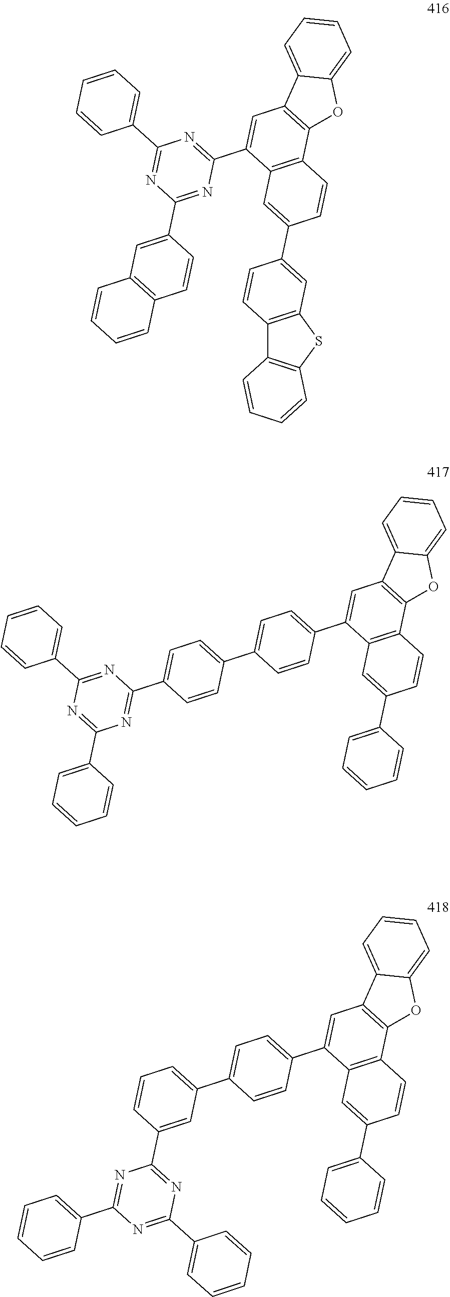 [SMILES: c1ccc(-c2nc(-c3ccc4ccccc4c3)nc(-c3cc4c5ccccc5oc4c4ccc(-c5ccc6c(c5)sc5ccccc56)cc34)n2)cc1 ; c1ccc(-c2ccc3c(c2)c(-c2ccc(-c4ccc(-c5nc(-c6ccccc6)nc(-c6ccccc6)n5)cc4)cc2)cc2c4ccccc4oc32)cc1 ; c1ccc(-c2ccc3c(c2)c(-c2ccc(-c4cccc(-c5nc(-c6ccccc6)nc(-c6ccccc6)n5)c4)cc2)cc2c4ccccc4oc32)cc1]

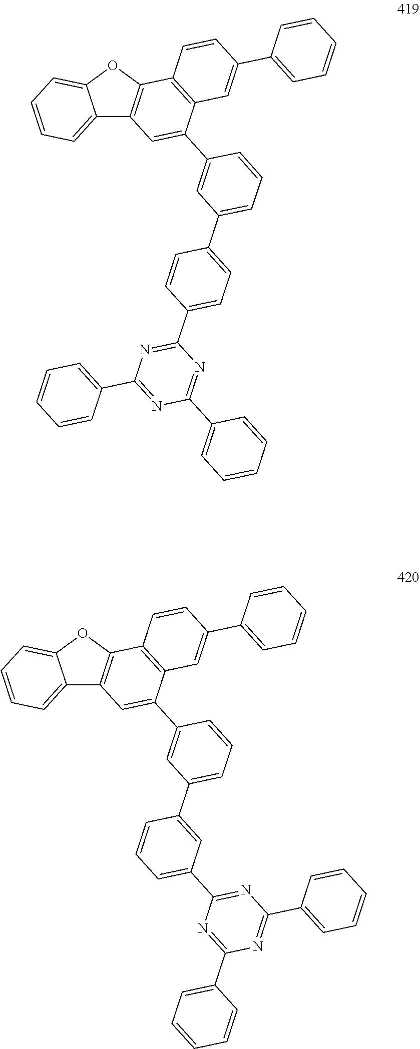 [SMILES: c1ccc(-c2ccc3c(c2)c(-c2cccc(-c4ccc(-c5nc(-c6ccccc6)nc(-c6ccccc6)n5)cc4)c2)cc2c4ccccc4oc32)cc1 ; c1ccc(-c2ccc3c(c2)c(-c2cccc(-c4cccc(-c5nc(-c6ccccc6)nc(-c6ccccc6)n5)c4)c2)cc2c4ccccc4oc32)cc1]

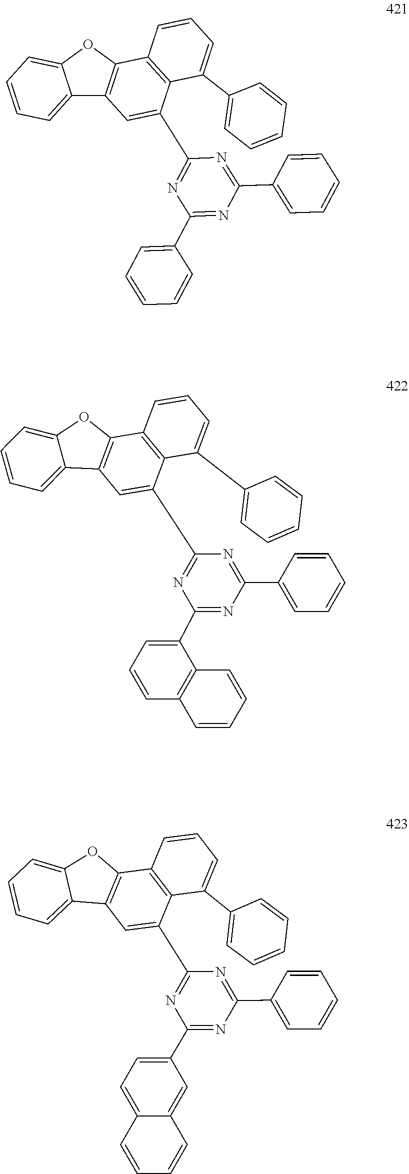 [SMILES: c1ccc(-c2nc(-c3ccccc3)nc(-c3cc4c5ccccc5oc4c4cccc(-c5ccccc5)c34)n2)cc1 ; c1ccc(-c2nc(-c3cccc4ccccc34)nc(-c3cc4c5ccccc5oc4c4cccc(-c5ccccc5)c34)n2)cc1 ; c1ccc(-c2nc(-c3ccc4ccccc4c3)nc(-c3cc4c5ccccc5oc4c4cccc(-c5ccccc5)c34)n2)cc1]

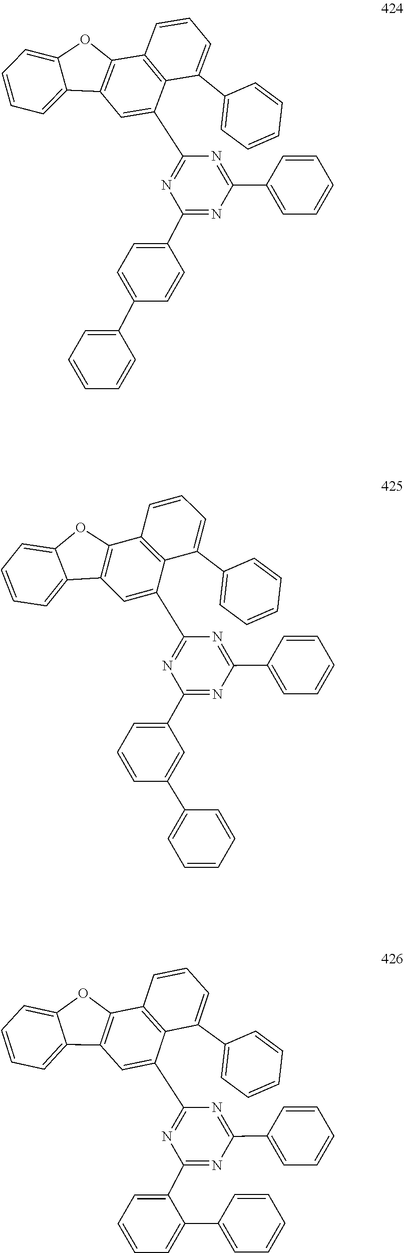 [SMILES: c1ccc(-c2ccc(-c3nc(-c4ccccc4)nc(-c4cc5c6ccccc6oc5c5cccc(-c6ccccc6)c45)n3)cc2)cc1 ; c1ccc(-c2cccc(-c3nc(-c4ccccc4)nc(-c4cc5c6ccccc6oc5c5cccc(-c6ccccc6)c45)n3)c2)cc1 ; c1ccc(-c2nc(-c3ccccc3-c3ccccc3)nc(-c3cc4c5ccccc5oc4c4cccc(-c5ccccc5)c34)n2)cc1]

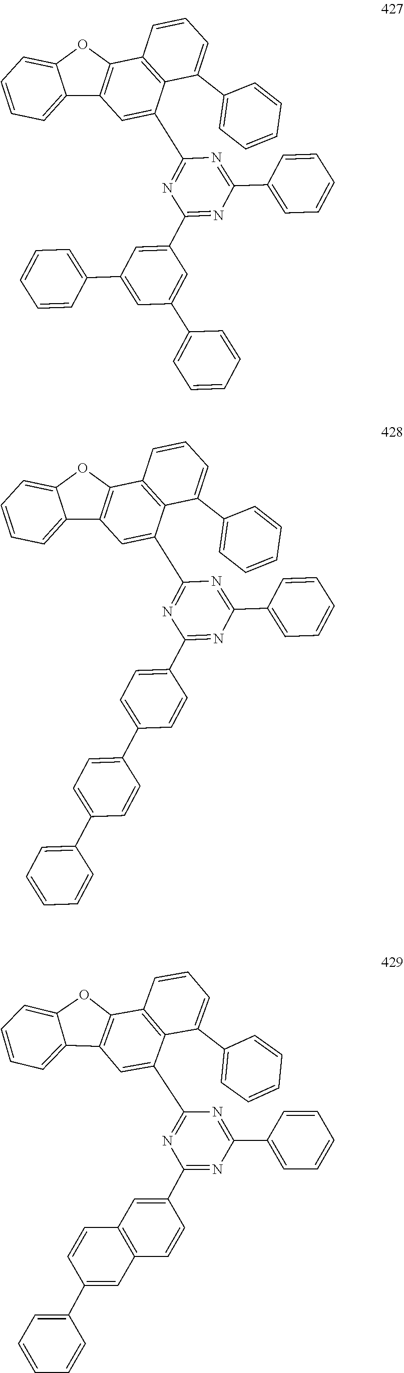 [SMILES: c1ccc(-c2cc(-c3ccccc3)cc(-c3nc(-c4ccccc4)nc(-c4cc5c6ccccc6oc5c5cccc(-c6ccccc6)c45)n3)c2)cc1 ; c1ccc(-c2ccc(-c3ccc(-c4nc(-c5ccccc5)nc(-c5cc6c7ccccc7oc6c6cccc(-c7ccccc7)c56)n4)cc3)cc2)cc1 ; c1ccc(-c2ccc3cc(-c4nc(-c5ccccc5)nc(-c5cc6c7ccccc7oc6c6cccc(-c7ccccc7)c56)n4)ccc3c2)cc1]

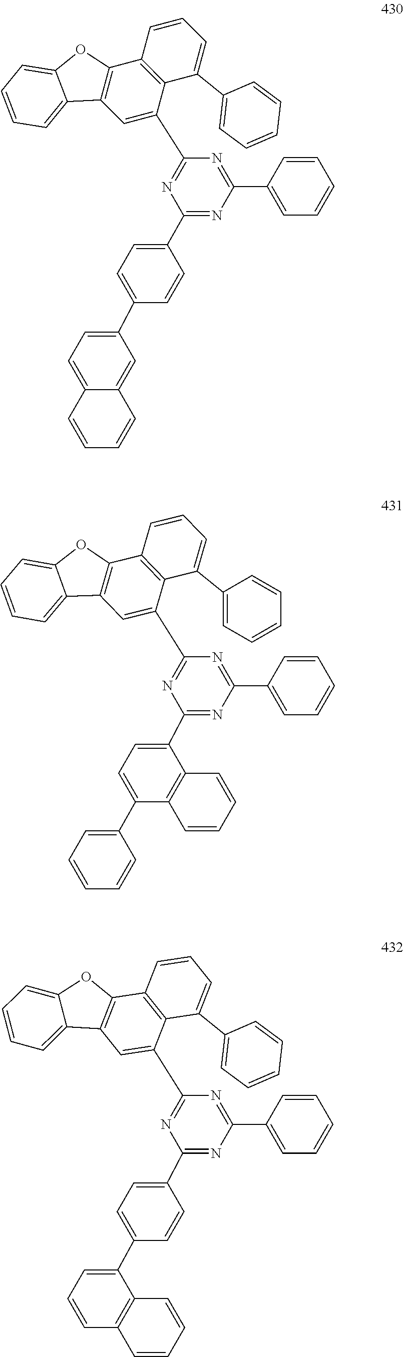 [SMILES: c1ccc(-c2nc(-c3ccc(-c4ccc5ccccc5c4)cc3)nc(-c3cc4c5ccccc5oc4c4cccc(-c5ccccc5)c34)n2)cc1 ; c1ccc(-c2nc(-c3ccc(-c4ccccc4)c4ccccc34)nc(-c3cc4c5ccccc5oc4c4cccc(-c5ccccc5)c34)n2)cc1 ; c1ccc(-c2nc(-c3ccc(-c4cccc5ccccc45)cc3)nc(-c3cc4c5ccccc5oc4c4cccc(-c5ccccc5)c34)n2)cc1]

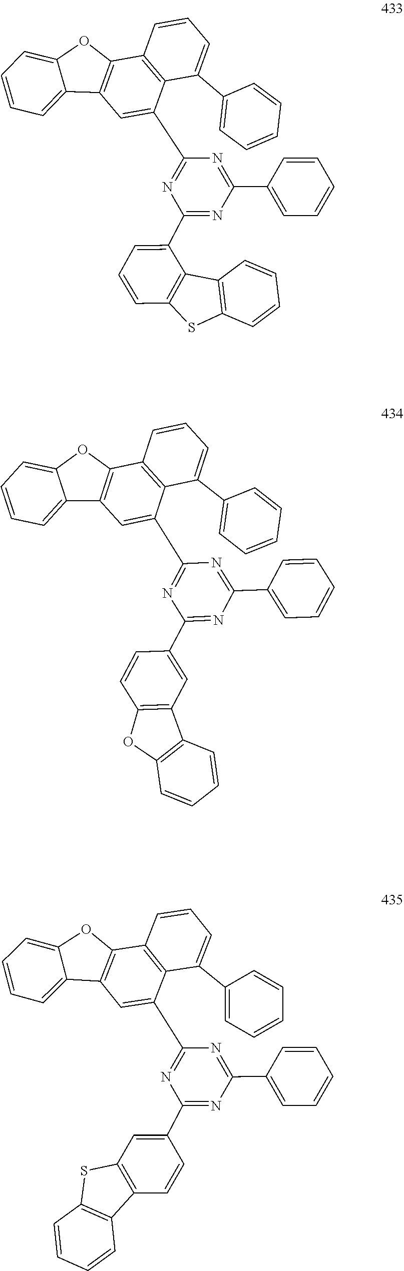 [SMILES: c1ccc(-c2nc(-c3cc4c5ccccc5oc4c4cccc(-c5ccccc5)c34)nc(-c3cccc4sc5ccccc5c34)n2)cc1 ; c1ccc(-c2nc(-c3ccc4oc5ccccc5c4c3)nc(-c3cc4c5ccccc5oc4c4cccc(-c5ccccc5)c34)n2)cc1 ; c1ccc(-c2nc(-c3ccc4c(c3)sc3ccccc34)nc(-c3cc4c5ccccc5oc4c4cccc(-c5ccccc5)c34)n2)cc1]

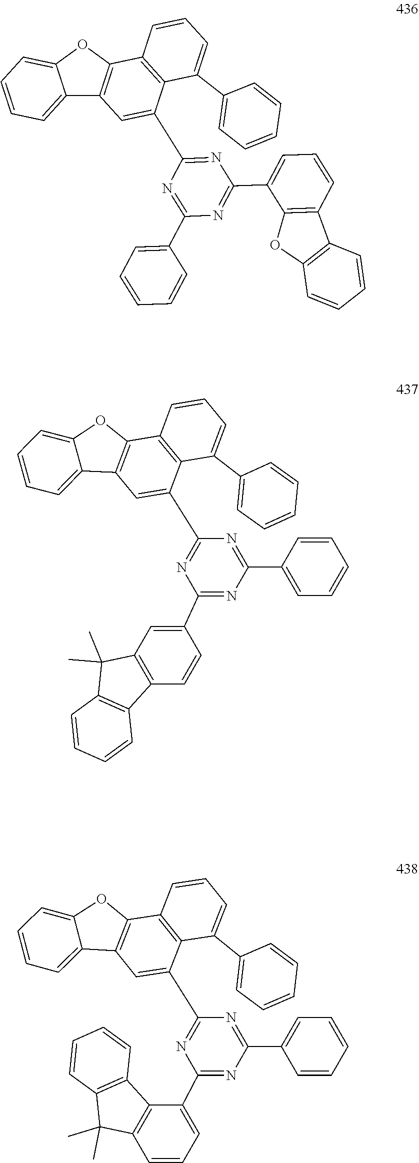 [SMILES: c1ccc(-c2nc(-c3cccc4c3oc3ccccc34)nc(-c3cc4c5ccccc5oc4c4cccc(-c5ccccc5)c34)n2)cc1 ; CC1(C)c2ccccc2-c2ccc(-c3nc(-c4ccccc4)nc(-c4cc5c6ccccc6oc5c5cccc(-c6ccccc6)c45)n3)cc21 ; CC1(C)c2ccccc2-c2c(-c3nc(-c4ccccc4)nc(-c4cc5c6ccccc6oc5c5cccc(-c6ccccc6)c45)n3)cccc21]

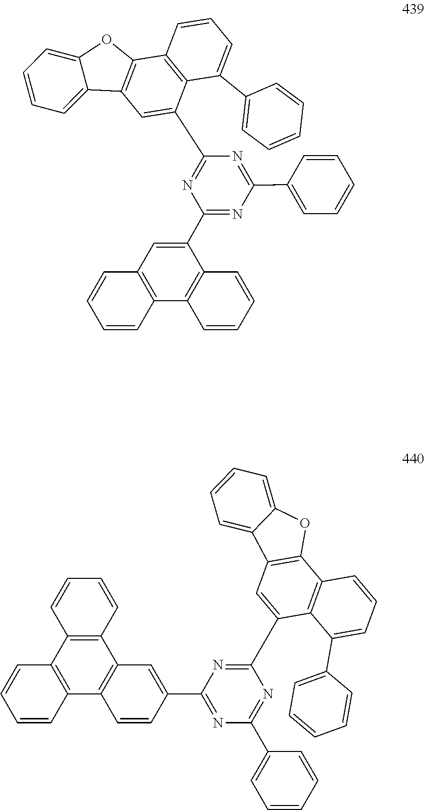 [SMILES: c1ccc(-c2nc(-c3cc4ccccc4c4ccccc34)nc(-c3cc4c5ccccc5oc4c4cccc(-c5ccccc5)c34)n2)cc1 ; c1ccc(-c2nc(-c3ccc4c5ccccc5c5ccccc5c4c3)nc(-c3cc4c5ccccc5oc4c4cccc(-c5ccccc5)c34)n2)cc1]

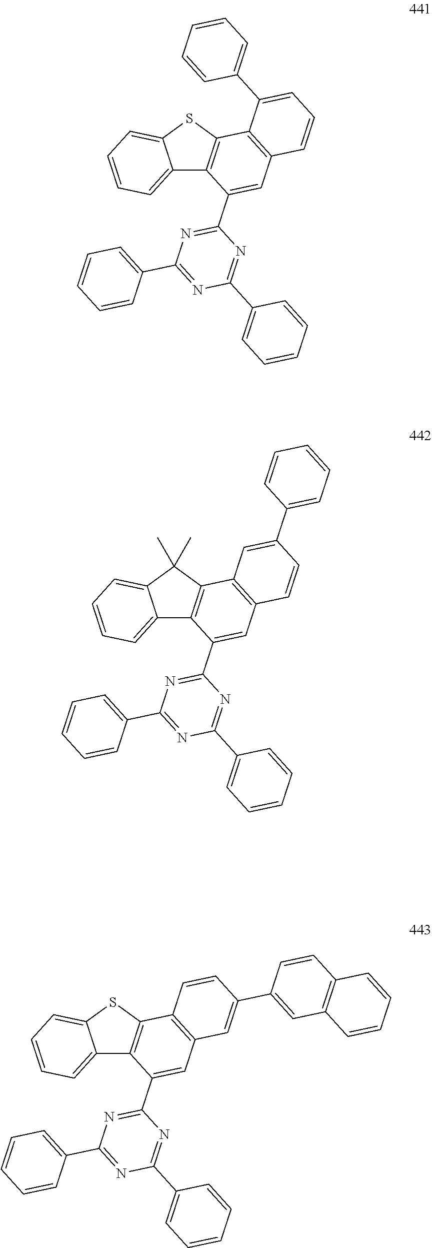 [SMILES: c1ccc(-c2nc(-c3ccccc3)nc(-c3cc4cccc(-c5ccccc5)c4c4sc5ccccc5c34)n2)cc1 ; CC1(C)c2ccccc2-c2c(-c3nc(-c4ccccc4)nc(-c4ccccc4)n3)cc3ccc(-c4ccccc4)cc3c21 ; c1ccc(-c2nc(-c3ccccc3)nc(-c3cc4cc(-c5ccc6ccccc6c5)ccc4c4sc5ccccc5c34)n2)cc1]

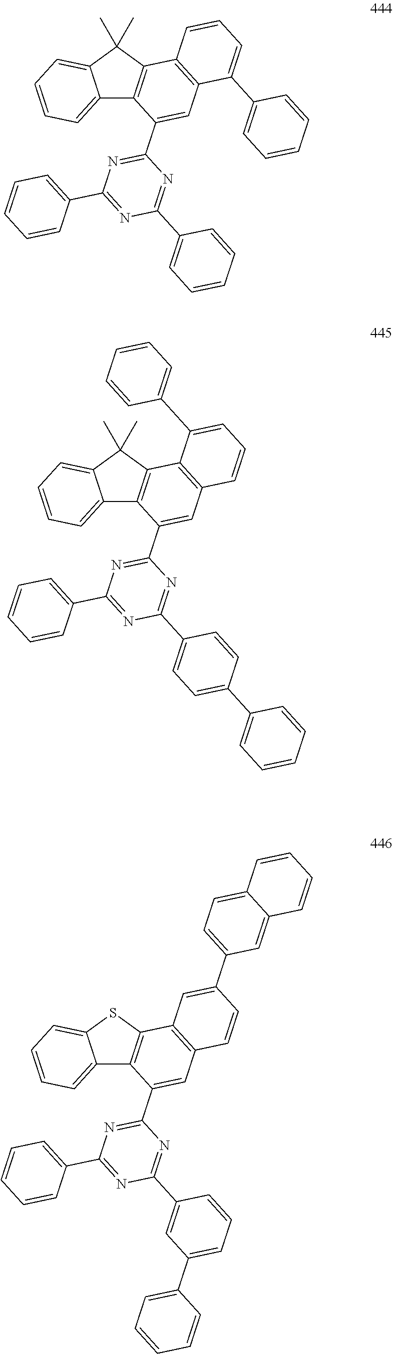 [SMILES: CC1(C)c2ccccc2-c2c(-c3nc(-c4ccccc4)nc(-c4ccccc4)n3)cc3c(-c4ccccc4)cccc3c21 ; CC1(C)c2ccccc2-c2c(-c3nc(-c4ccccc4)nc(-c4ccc(-c5ccccc5)cc4)n3)cc3cccc(-c4ccccc4)c3c21 ; c1ccc(-c2cccc(-c3nc(-c4ccccc4)nc(-c4cc5ccc(-c6ccc7ccccc7c6)cc5c5sc6ccccc6c45)n3)c2)cc1]

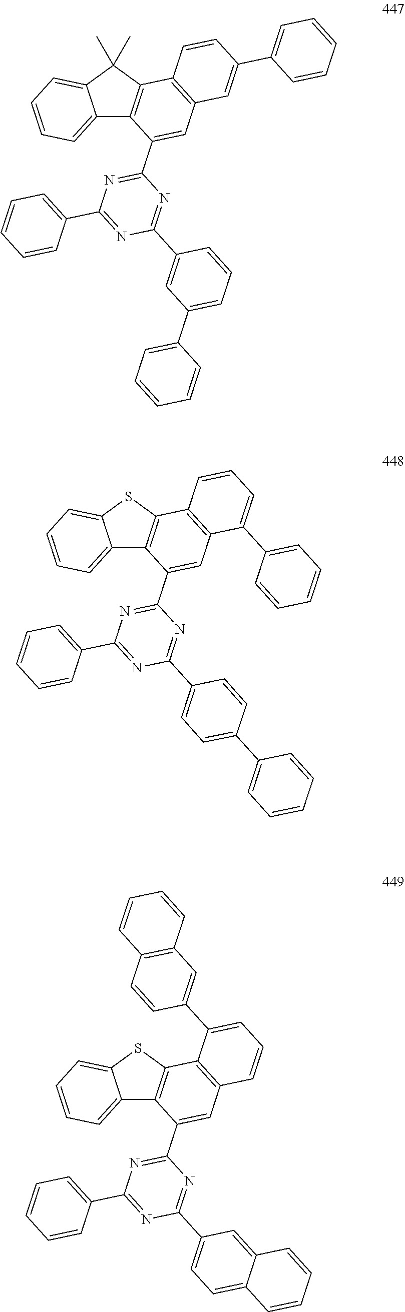 [SMILES: CC1(C)c2ccccc2-c2c(-c3nc(-c4ccccc4)nc(-c4cccc(-c5ccccc5)c4)n3)cc3cc(-c4ccccc4)ccc3c21 ; c1ccc(-c2ccc(-c3nc(-c4ccccc4)nc(-c4cc5c(-c6ccccc6)cccc5c5sc6ccccc6c45)n3)cc2)cc1 ; c1ccc(-c2nc(-c3ccc4ccccc4c3)nc(-c3cc4cccc(-c5ccc6ccccc6c5)c4c4sc5ccccc5c34)n2)cc1]

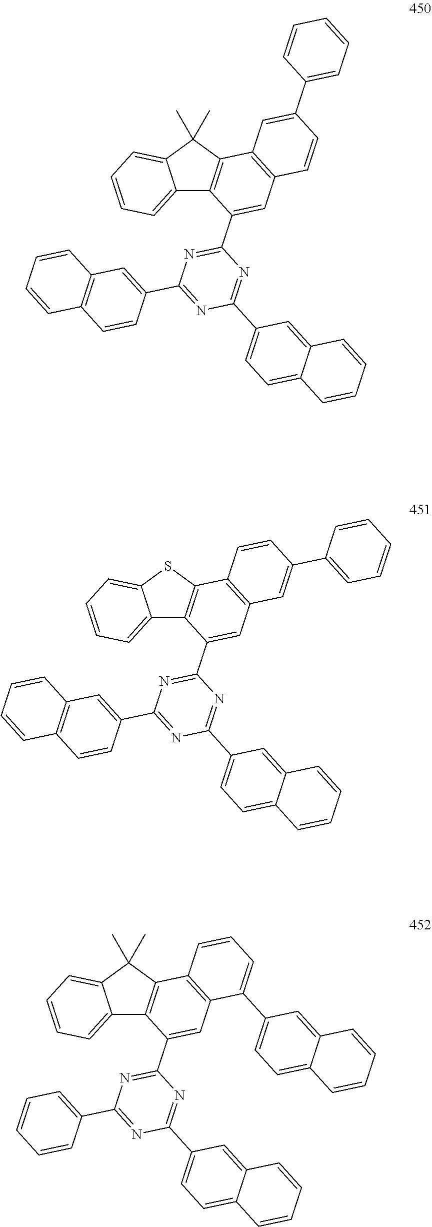 [SMILES: CC1(C)c2ccccc2-c2c(-c3nc(-c4ccc5ccccc5c4)nc(-c4ccc5ccccc5c4)n3)cc3ccc(-c4ccccc4)cc3c21 ; c1ccc(-c2ccc3c(c2)cc(-c2nc(-c4ccc5ccccc5c4)nc(-c4ccc5ccccc5c4)n2)c2c4ccccc4sc32)cc1 ; CC1(C)c2ccccc2-c2c(-c3nc(-c4ccccc4)nc(-c4ccc5ccccc5c4)n3)cc3c(-c4ccc5ccccc5c4)cccc3c21]

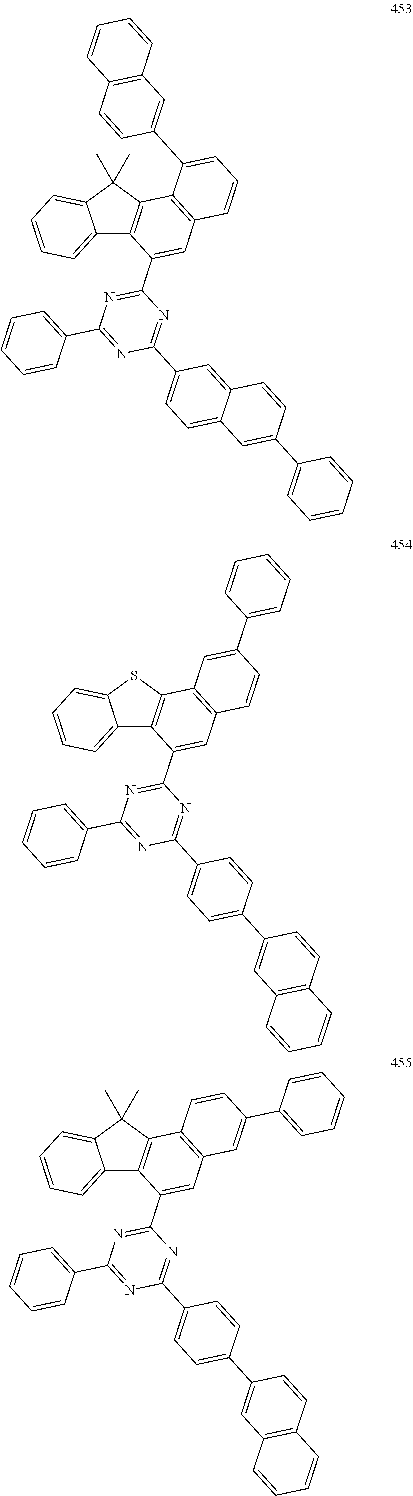

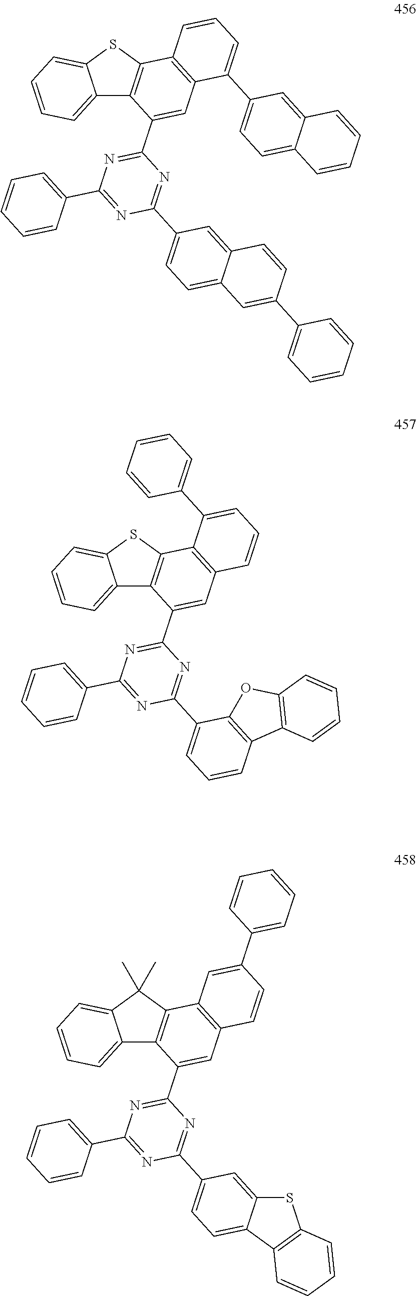 [SMILES: c1ccc(-c2ccc3cc(-c4nc(-c5ccccc5)nc(-c5cc6c(-c7ccc8ccccc8c7)cccc6c6sc7ccccc7c56)n4)ccc3c2)cc1 ; c1ccc(-c2nc(-c3cccc4c3oc3ccccc34)nc(-c3cc4cccc(-c5ccccc5)c4c4sc5ccccc5c34)n2)cc1 ; CC1(C)c2ccccc2-c2c(-c3nc(-c4ccccc4)nc(-c4ccc5c(c4)sc4ccccc45)n3)cc3ccc(-c4ccccc4)cc3c21]

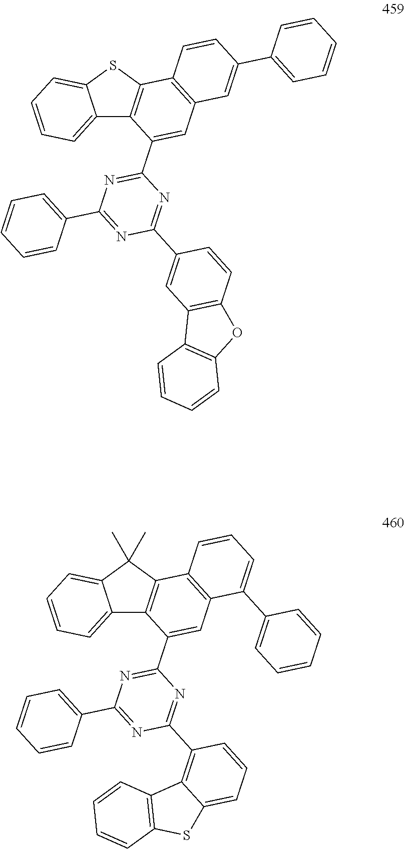 [SMILES: c1ccc(-c2ccc3c(c2)cc(-c2nc(-c4ccccc4)nc(-c4ccc5oc6ccccc6c5c4)n2)c2c4ccccc4sc32)cc1 ; CC1(C)c2ccccc2-c2c(-c3nc(-c4ccccc4)nc(-c4cccc5sc6ccccc6c45)n3)cc3c(-c4ccccc4)cccc3c21]

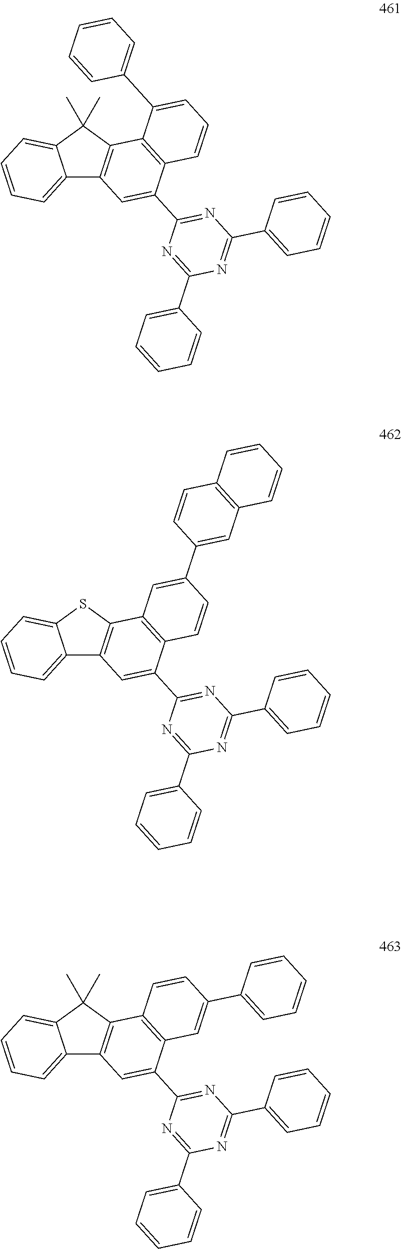 [SMILES: CC1(C)c2ccccc2-c2cc(-c3nc(-c4ccccc4)nc(-c4ccccc4)n3)c3cccc(-c4ccccc4)c3c21 ; c1ccc(-c2nc(-c3ccccc3)nc(-c3cc4c5ccccc5sc4c4cc(-c5ccc6ccccc6c5)ccc34)n2)cc1 ; CC1(C)c2ccccc2-c2cc(-c3nc(-c4ccccc4)nc(-c4ccccc4)n3)c3cc(-c4ccccc4)ccc3c21]

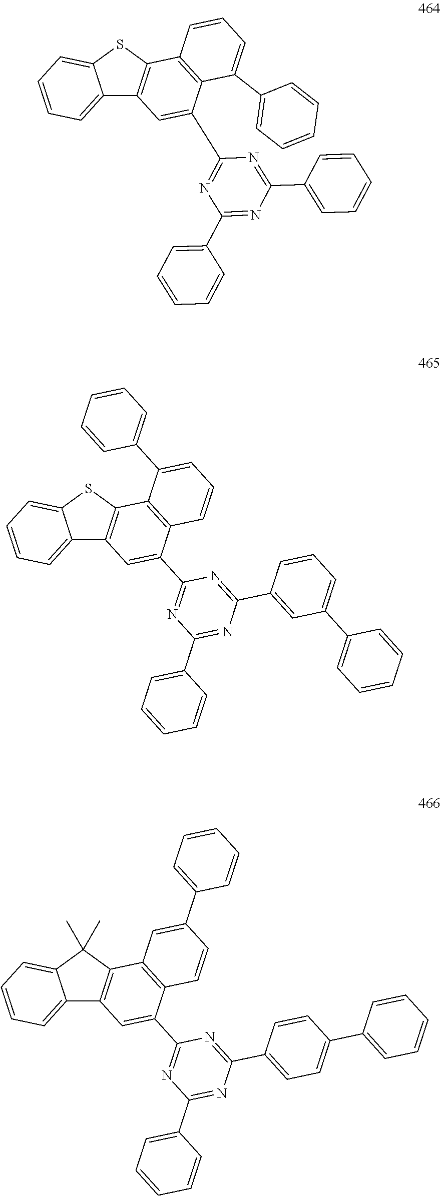 [SMILES: c1ccc(-c2nc(-c3ccccc3)nc(-c3cc4c5ccccc5sc4c4cccc(-c5ccccc5)c34)n2)cc1 ; c1ccc(-c2cccc(-c3nc(-c4ccccc4)nc(-c4cc5c6ccccc6sc5c5c(-c6ccccc6)cccc45)n3)c2)cc1 ; CC1(C)c2ccccc2-c2cc(-c3nc(-c4ccccc4)nc(-c4ccc(-c5ccccc5)cc4)n3)c3ccc(-c4ccccc4)cc3c21]

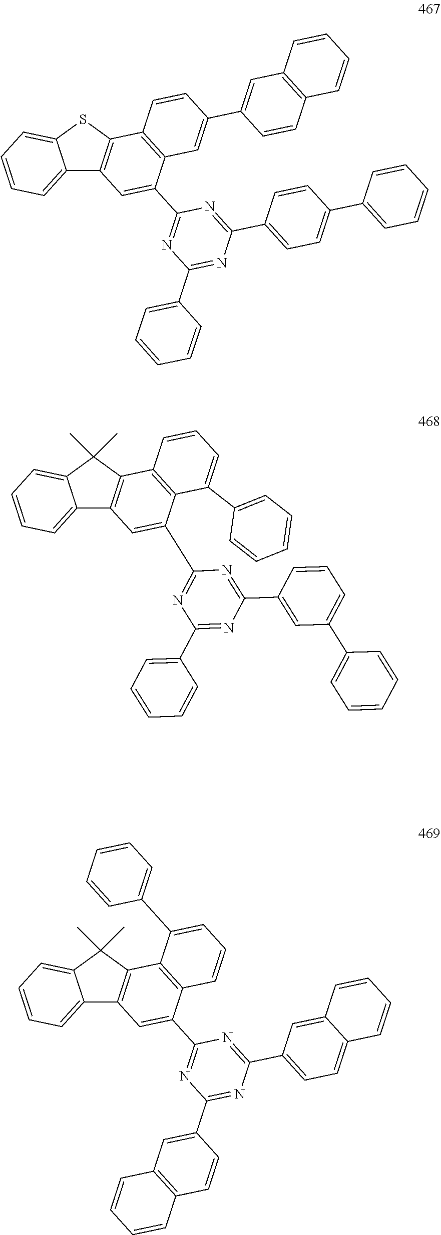 [SMILES: c1ccc(-c2ccc(-c3nc(-c4ccccc4)nc(-c4cc5c6ccccc6sc5c5ccc(-c6ccc7ccccc7c6)cc45)n3)cc2)cc1 ; CC1(C)c2ccccc2-c2cc(-c3nc(-c4ccccc4)nc(-c4cccc(-c5ccccc5)c4)n3)c3c(-c4ccccc4)cccc3c21 ; CC1(C)c2ccccc2-c2cc(-c3nc(-c4ccc5ccccc5c4)nc(-c4ccc5ccccc5c4)n3)c3cccc(-c4ccccc4)c3c21]

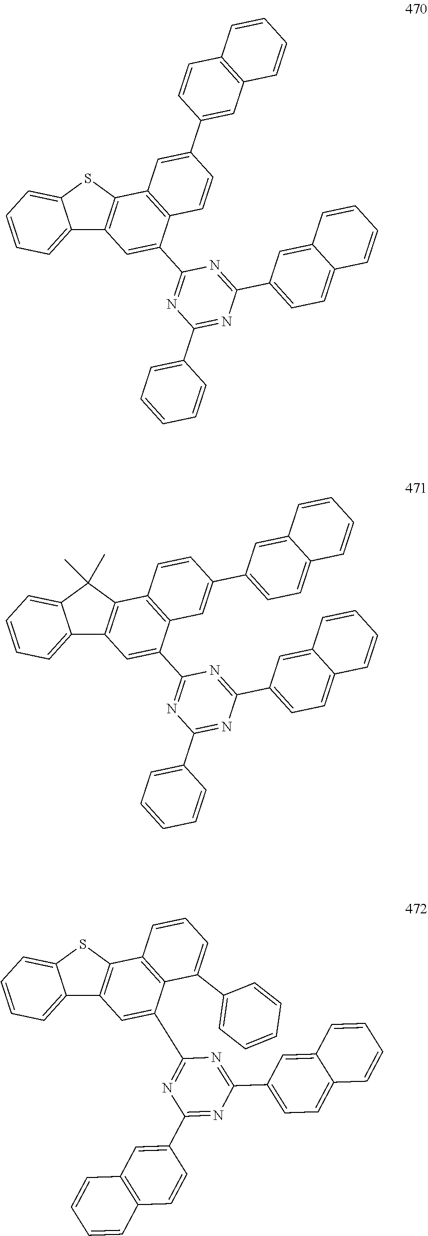 [SMILES: c1ccc(-c2nc(-c3ccc4ccccc4c3)nc(-c3cc4c5ccccc5sc4c4cc(-c5ccc6ccccc6c5)ccc34)n2)cc1 ; CC1(C)c2ccccc2-c2cc(-c3nc(-c4ccccc4)nc(-c4ccc5ccccc5c4)n3)c3cc(-c4ccc5ccccc5c4)ccc3c21 ; c1ccc(-c2cccc3c2c(-c2nc(-c4ccc5ccccc5c4)nc(-c4ccc5ccccc5c4)n2)cc2c4ccccc4sc32)cc1]

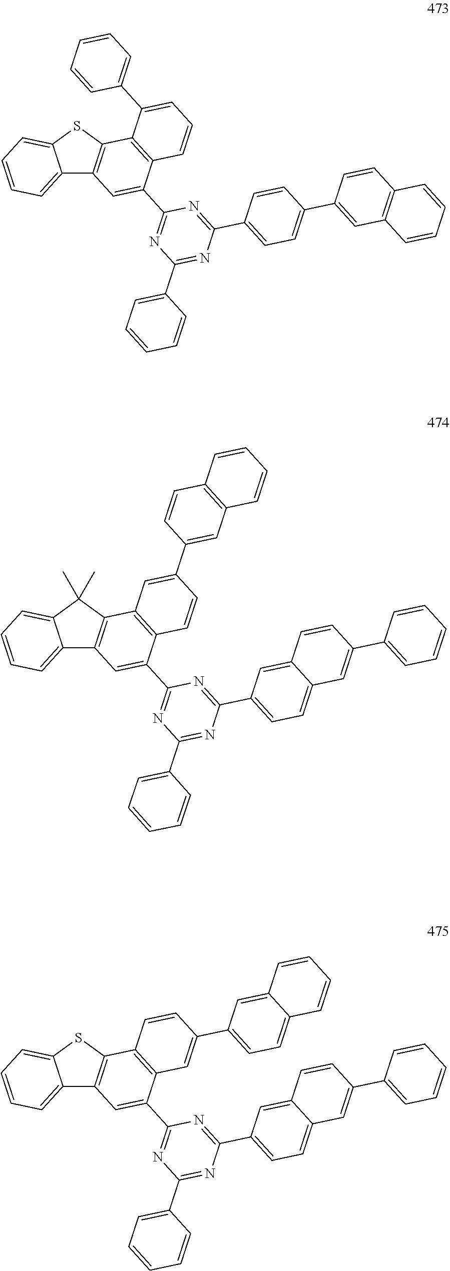 [SMILES: c1ccc(-c2nc(-c3ccc(-c4ccc5ccccc5c4)cc3)nc(-c3cc4c5ccccc5sc4c4c(-c5ccccc5)cccc34)n2)cc1 ; CC1(C)c2ccccc2-c2cc(-c3nc(-c4ccccc4)nc(-c4ccc5cc(-c6ccccc6)ccc5c4)n3)c3ccc(-c4ccc5ccccc5c4)cc3c21 ; c1ccc(-c2ccc3cc(-c4nc(-c5ccccc5)nc(-c5cc6c7ccccc7sc6c6ccc(-c7ccc8ccccc8c7)cc56)n4)ccc3c2)cc1]

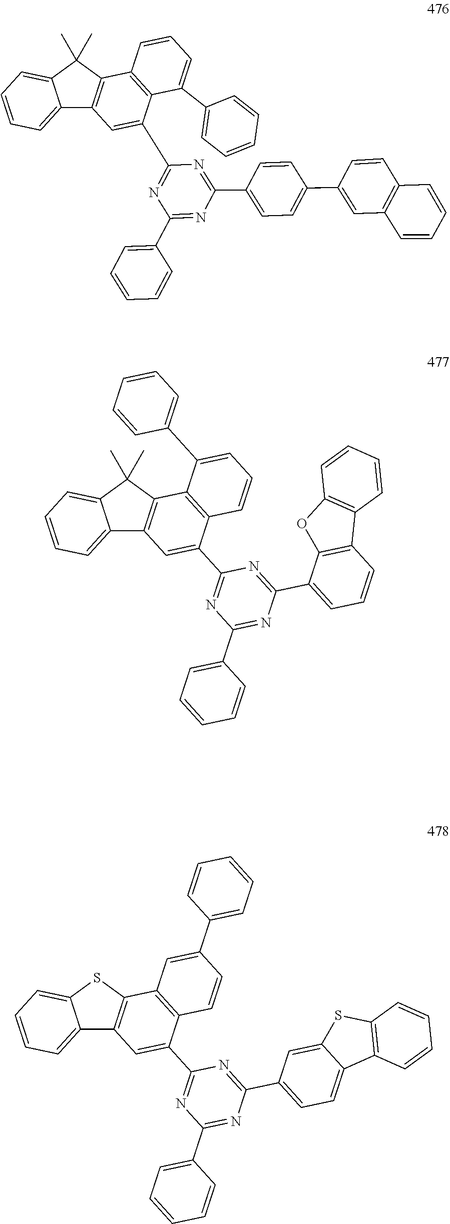 [SMILES: CC1(C)c2ccccc2-c2cc(-c3nc(-c4ccccc4)nc(-c4ccc(-c5ccc6ccccc6c5)cc4)n3)c3c(-c4ccccc4)cccc3c21 ; CC1(C)c2ccccc2-c2cc(-c3nc(-c4ccccc4)nc(-c4cccc5c4oc4ccccc45)n3)c3cccc(-c4ccccc4)c3c21 ; c1ccc(-c2ccc3c(-c4nc(-c5ccccc5)nc(-c5ccc6c(c5)sc5ccccc56)n4)cc4c5ccccc5sc4c3c2)cc1]

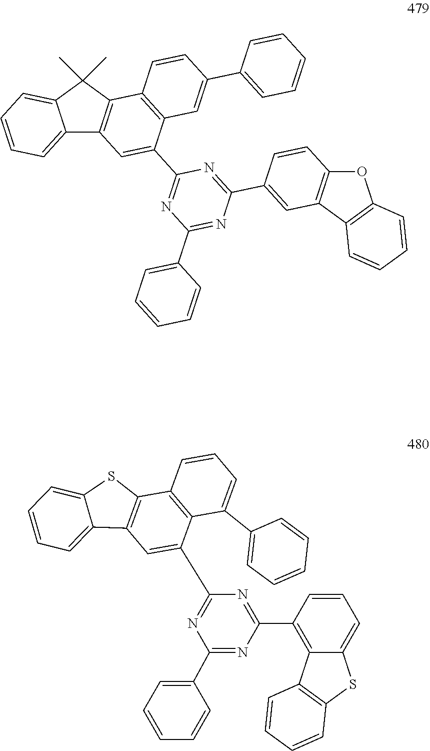 [SMILES: CC1(C)c2ccccc2-c2cc(-c3nc(-c4ccccc4)nc(-c4ccc5oc6ccccc6c5c4)n3)c3cc(-c4ccccc4)ccc3c21 ; c1ccc(-c2nc(-c3cc4c5ccccc5sc4c4cccc(-c5ccccc5)c34)nc(-c3cccc4sc5ccccc5c34)n2)cc1]

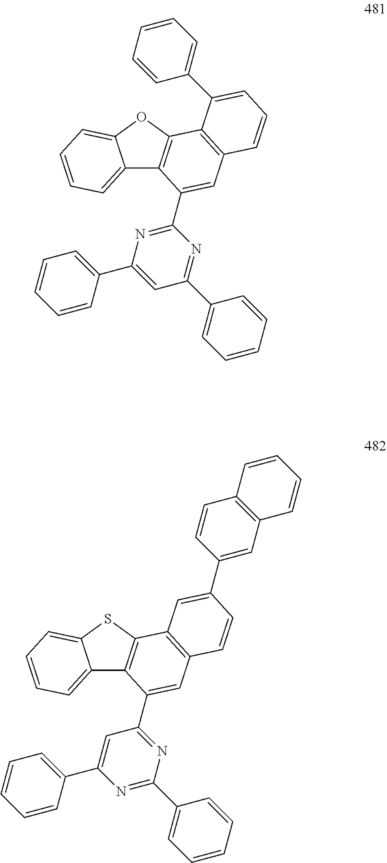 [SMILES: c1ccc(-c2cc(-c3ccccc3)nc(-c3cc4cccc(-c5ccccc5)c4c4oc5ccccc5c34)n2)cc1 ; c1ccc(-c2cc(-c3cc4ccc(-c5ccc6ccccc6c5)cc4c4sc5ccccc5c34)nc(-c3ccccc3)n2)cc1]

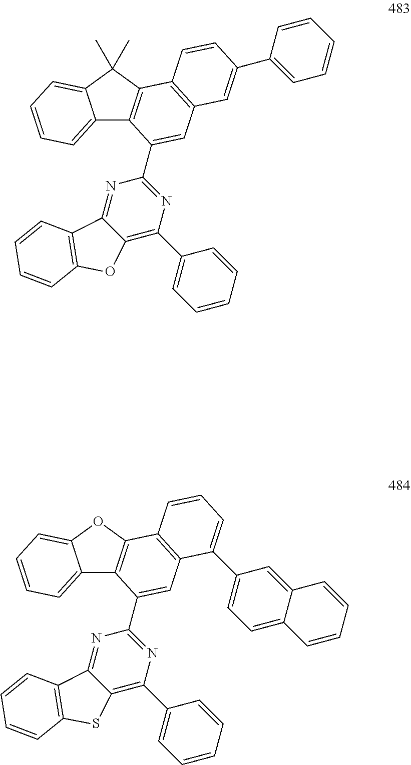 [SMILES: CC1(C)c2ccccc2-c2c(-c3nc(-c4ccccc4)c4oc5ccccc5c4n3)cc3cc(-c4ccccc4)ccc3c21 ; c1ccc(-c2nc(-c3cc4c(-c5ccc6ccccc6c5)cccc4c4oc5ccccc5c34)nc3c2sc2ccccc23)cc1]

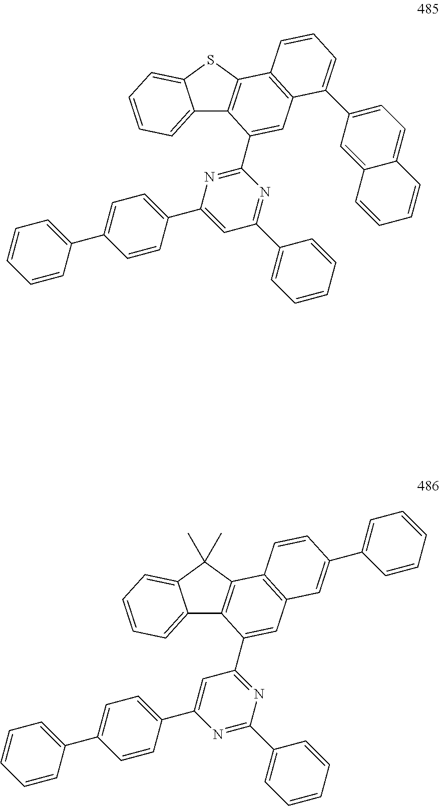 [SMILES: c1ccc(-c2ccc(-c3cc(-c4ccccc4)nc(-c4cc5c(-c6ccc7ccccc7c6)cccc5c5sc6ccccc6c45)n3)cc2)cc1 ; CC1(C)c2ccccc2-c2c(-c3cc(-c4ccc(-c5ccccc5)cc4)nc(-c4ccccc4)n3)cc3cc(-c4ccccc4)ccc3c21]

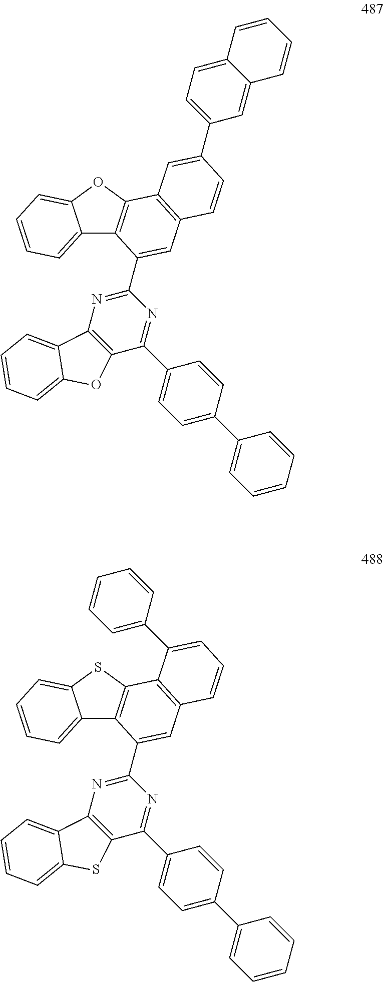 [SMILES: c1ccc(-c2ccc(-c3nc(-c4cc5ccc(-c6ccc7ccccc7c6)cc5c5oc6ccccc6c45)nc4c3oc3ccccc34)cc2)cc1 ; c1ccc(-c2ccc(-c3nc(-c4cc5cccc(-c6ccccc6)c5c5sc6ccccc6c45)nc4c3sc3ccccc34)cc2)cc1]

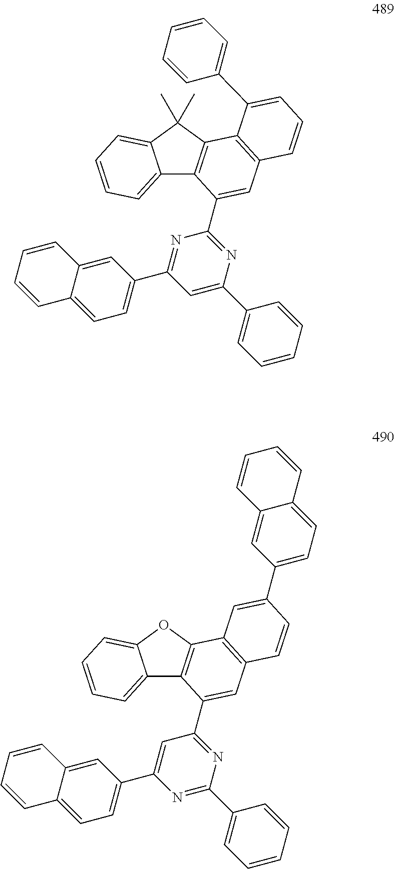 [SMILES: CC1(C)c2ccccc2-c2c(-c3nc(-c4ccccc4)cc(-c4ccc5ccccc5c4)n3)cc3cccc(-c4ccccc4)c3c21 ; c1ccc(-c2nc(-c3ccc4ccccc4c3)cc(-c3cc4ccc(-c5ccc6ccccc6c5)cc4c4oc5ccccc5c34)n2)cc1]

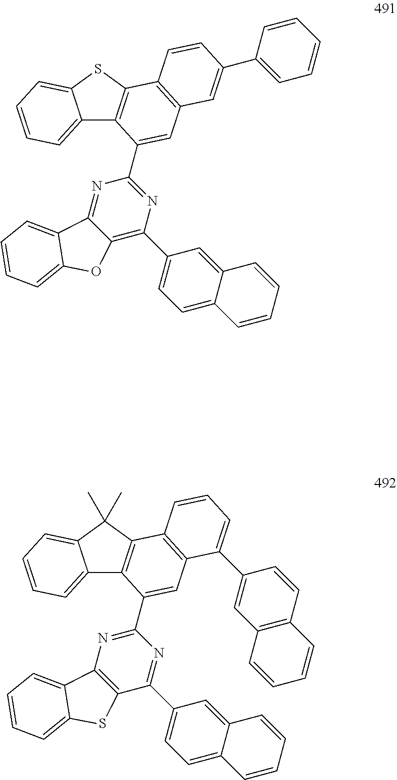 [SMILES: c1ccc(-c2ccc3c(c2)cc(-c2nc(-c4ccc5ccccc5c4)c4oc5ccccc5c4n2)c2c4ccccc4sc32)cc1 ; CC1(C)c2ccccc2-c2c(-c3nc(-c4ccc5ccccc5c4)c4sc5ccccc5c4n3)cc3c(-c4ccc5ccccc5c4)cccc3c21]

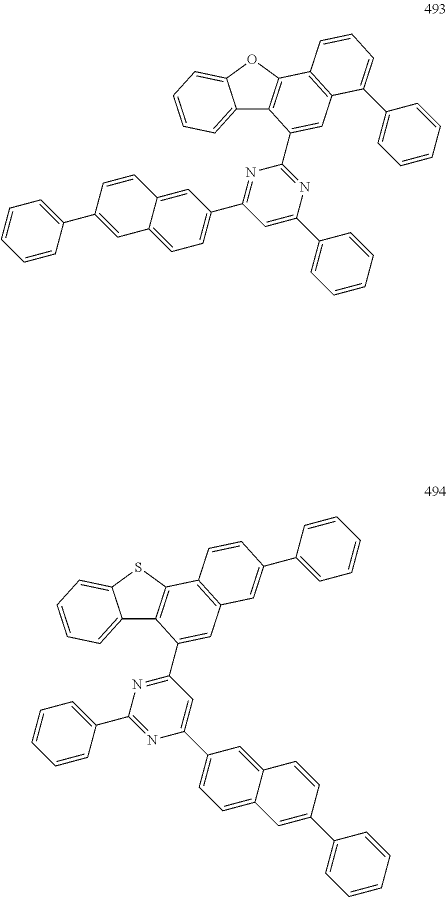 [SMILES: c1ccc(-c2ccc3cc(-c4cc(-c5ccccc5)nc(-c5cc6c(-c7ccccc7)cccc6c6oc7ccccc7c56)n4)ccc3c2)cc1 ; c1ccc(-c2ccc3cc(-c4cc(-c5cc6cc(-c7ccccc7)ccc6c6sc7ccccc7c56)nc(-c5ccccc5)n4)ccc3c2)cc1]

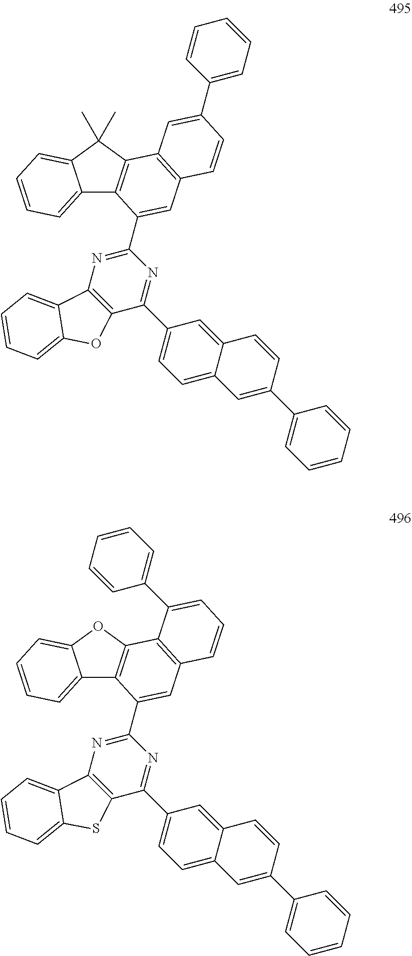 [SMILES: CC1(C)c2ccccc2-c2c(-c3nc(-c4ccc5cc(-c6ccccc6)ccc5c4)c4oc5ccccc5c4n3)cc3ccc(-c4ccccc4)cc3c21 ; c1ccc(-c2ccc3cc(-c4nc(-c5cc6cccc(-c7ccccc7)c6c6oc7ccccc7c56)nc5c4sc4ccccc45)ccc3c2)cc1]

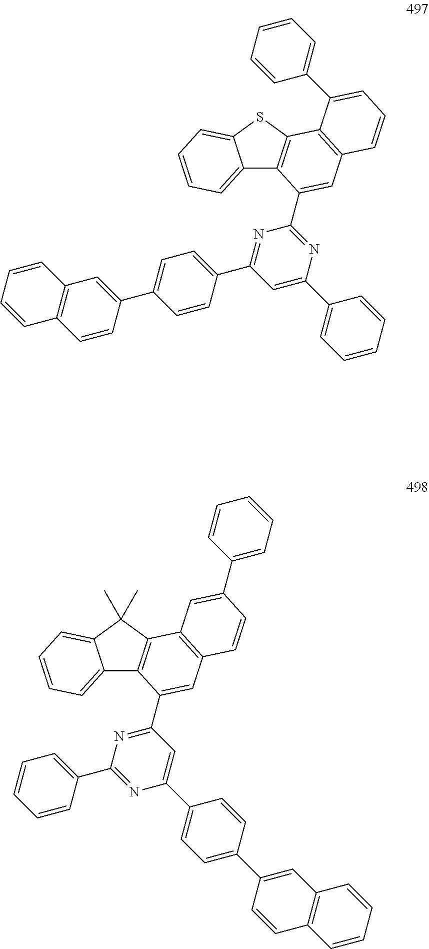 [SMILES: c1ccc(-c2cc(-c3ccc(-c4ccc5ccccc5c4)cc3)nc(-c3cc4cccc(-c5ccccc5)c4c4sc5ccccc5c34)n2)cc1 ; CC1(C)c2ccccc2-c2c(-c3cc(-c4ccc(-c5ccc6ccccc6c5)cc4)nc(-c4ccccc4)n3)cc3ccc(-c4ccccc4)cc3c21]

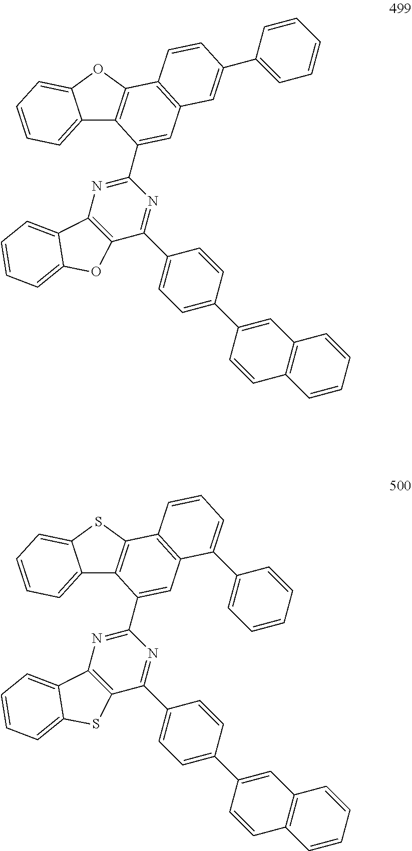 [SMILES: c1ccc(-c2ccc3c(c2)cc(-c2nc(-c4ccc(-c5ccc6ccccc6c5)cc4)c4oc5ccccc5c4n2)c2c4ccccc4oc32)cc1 ; c1ccc(-c2cccc3c2cc(-c2nc(-c4ccc(-c5ccc6ccccc6c5)cc4)c4sc5ccccc5c4n2)c2c4ccccc4sc32)cc1]

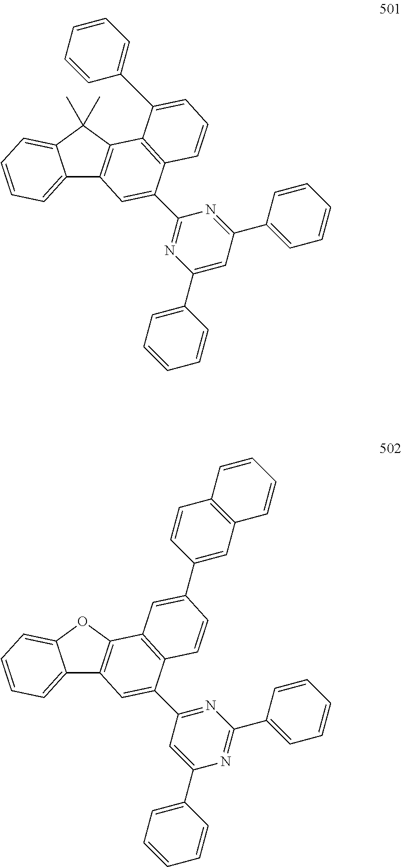 [SMILES: CC1(C)c2ccccc2-c2cc(-c3nc(-c4ccccc4)cc(-c4ccccc4)n3)c3cccc(-c4ccccc4)c3c21 ; c1ccc(-c2cc(-c3cc4c5ccccc5oc4c4cc(-c5ccc6ccccc6c5)ccc34)nc(-c3ccccc3)n2)cc1]

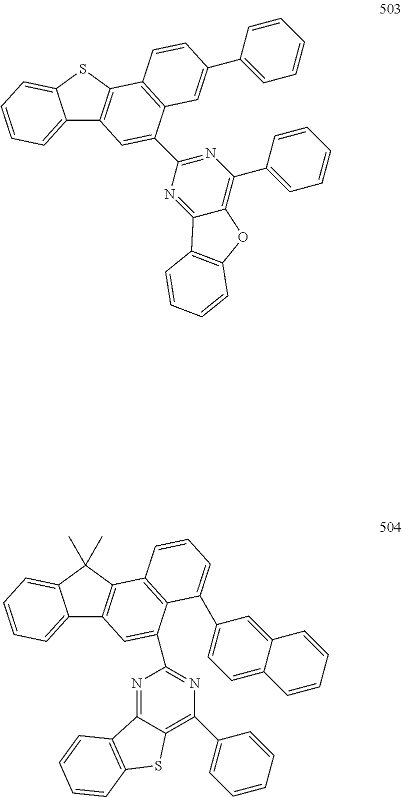 [SMILES: c1ccc(-c2ccc3c(c2)c(-c2nc(-c4ccccc4)c4oc5ccccc5c4n2)cc2c4ccccc4sc32)cc1 ; CC1(C)c2ccccc2-c2cc(-c3nc(-c4ccccc4)c4sc5ccccc5c4n3)c3c(-c4ccc5ccccc5c4)cccc3c21]

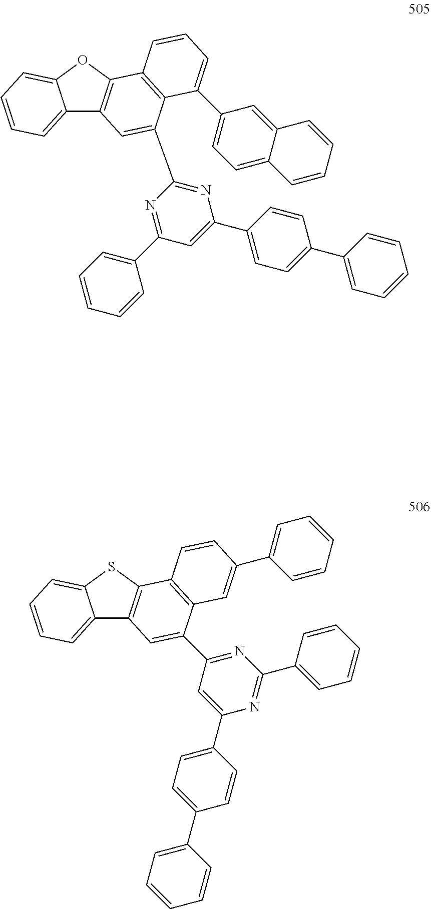 [SMILES: c1ccc(-c2ccc(-c3cc(-c4ccccc4)nc(-c4cc5c6ccccc6oc5c5cccc(-c6ccc7ccccc7c6)c45)n3)cc2)cc1 ; c1ccc(-c2ccc(-c3cc(-c4cc5c6ccccc6sc5c5ccc(-c6ccccc6)cc45)nc(-c4ccccc4)n3)cc2)cc1]

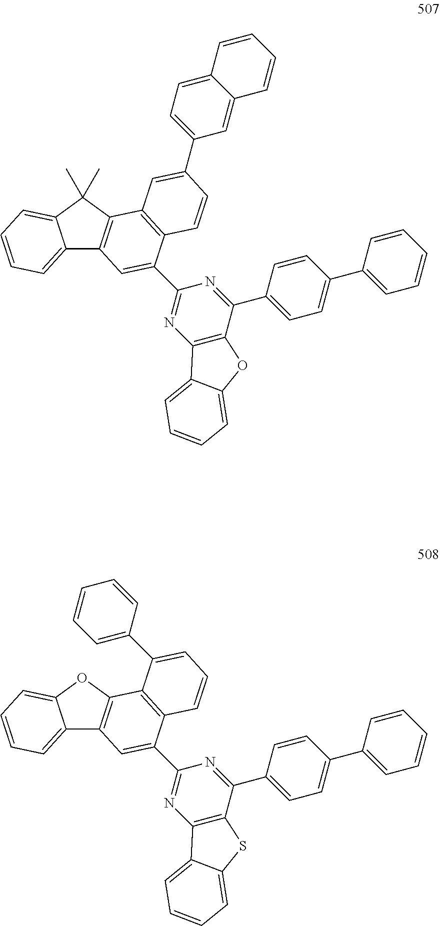 [SMILES: CC1(C)c2ccccc2-c2cc(-c3nc(-c4ccc(-c5ccccc5)cc4)c4oc5ccccc5c4n3)c3ccc(-c4ccc5ccccc5c4)cc3c21 ; c1ccc(-c2ccc(-c3nc(-c4cc5c6ccccc6oc5c5c(-c6ccccc6)cccc45)nc4c3sc3ccccc34)cc2)cc1]

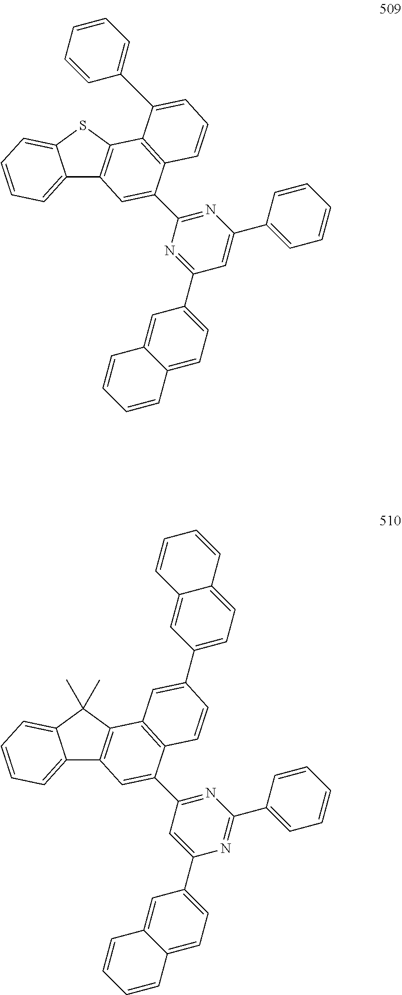 [SMILES: c1ccc(-c2cc(-c3ccc4ccccc4c3)nc(-c3cc4c5ccccc5sc4c4c(-c5ccccc5)cccc34)n2)cc1 ; CC1(C)c2ccccc2-c2cc(-c3cc(-c4ccc5ccccc5c4)nc(-c4ccccc4)n3)c3ccc(-c4ccc5ccccc5c4)cc3c21]

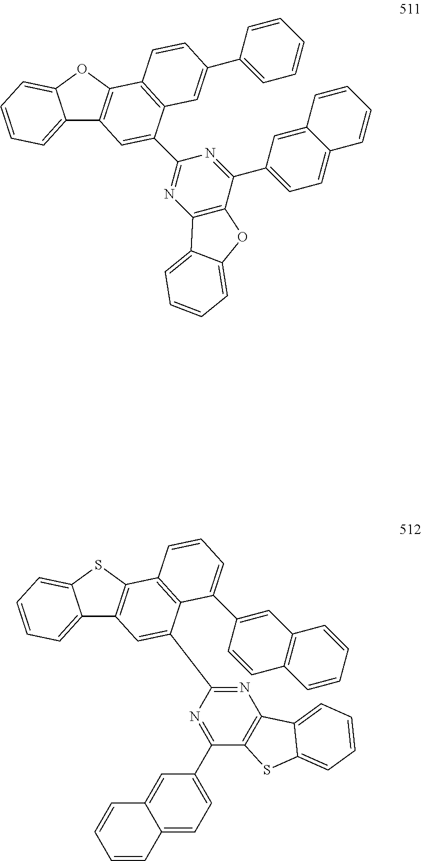 [SMILES: c1ccc(-c2ccc3c(c2)c(-c2nc(-c4ccc5ccccc5c4)c4oc5ccccc5c4n2)cc2c4ccccc4oc32)cc1 ; c1ccc2cc(-c3nc(-c4cc5c6ccccc6sc5c5cccc(-c6ccc7ccccc7c6)c45)nc4c3sc3ccccc34)ccc2c1]

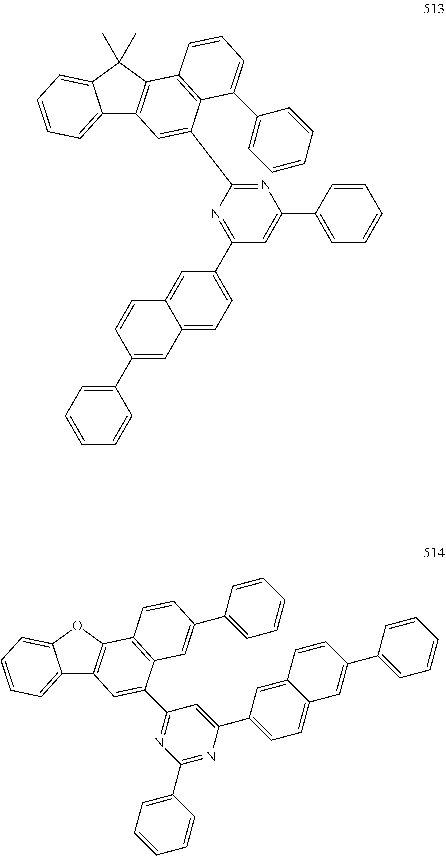 [SMILES: CC1(C)c2ccccc2-c2cc(-c3nc(-c4ccccc4)cc(-c4ccc5cc(-c6ccccc6)ccc5c4)n3)c3c(-c4ccccc4)cccc3c21 ; c1ccc(-c2ccc3cc(-c4cc(-c5cc6c7ccccc7oc6c6ccc(-c7ccccc7)cc56)nc(-c5ccccc5)n4)ccc3c2)cc1]

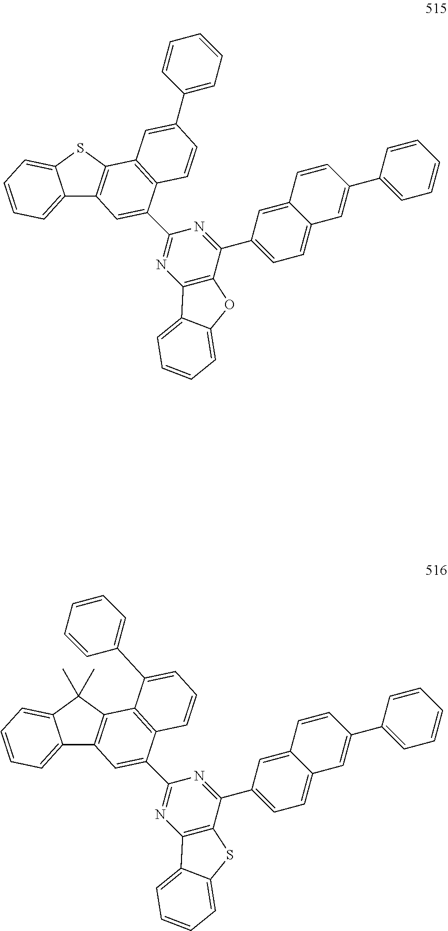 [SMILES: c1ccc(-c2ccc3cc(-c4nc(-c5cc6c7ccccc7sc6c6cc(-c7ccccc7)ccc56)nc5c4oc4ccccc45)ccc3c2)cc1 ; CC1(C)c2ccccc2-c2cc(-c3nc(-c4ccc5cc(-c6ccccc6)ccc5c4)c4sc5ccccc5c4n3)c3cccc(-c4ccccc4)c3c21]

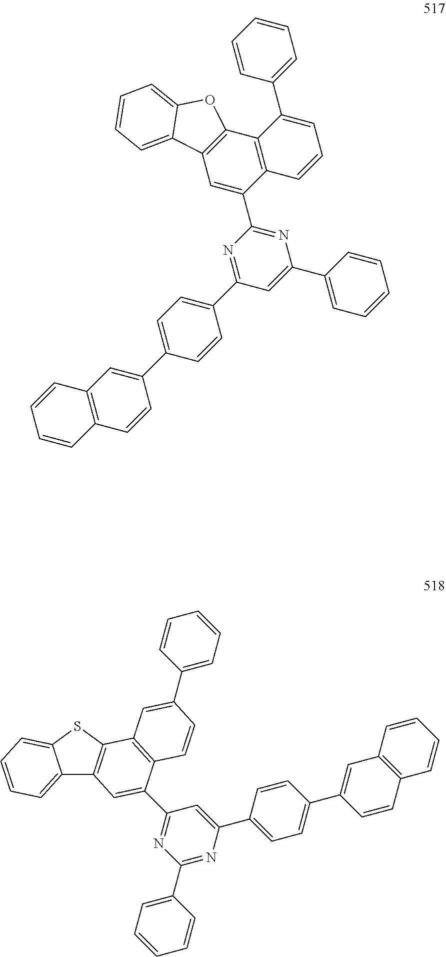 [SMILES: c1ccc(-c2cc(-c3ccc(-c4ccc5ccccc5c4)cc3)nc(-c3cc4c5ccccc5oc4c4c(-c5ccccc5)cccc34)n2)cc1 ; c1ccc(-c2ccc3c(-c4cc(-c5ccc(-c6ccc7ccccc7c6)cc5)nc(-c5ccccc5)n4)cc4c5ccccc5sc4c3c2)cc1]

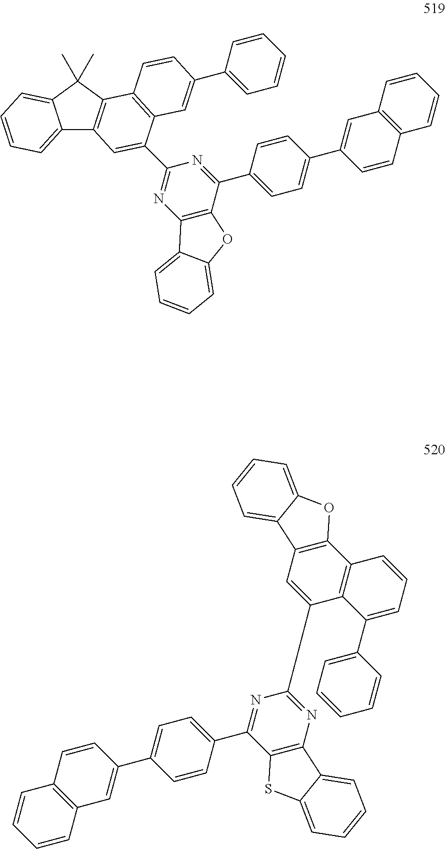 [SMILES: CC1(C)c2ccccc2-c2cc(-c3nc(-c4ccc(-c5ccc6ccccc6c5)cc4)c4oc5ccccc5c4n3)c3cc(-c4ccccc4)ccc3c21 ; c1ccc(-c2cccc3c2c(-c2nc(-c4ccc(-c5ccc6ccccc6c5)cc4)c4sc5ccccc5c4n2)cc2c4ccccc4oc32)cc1]

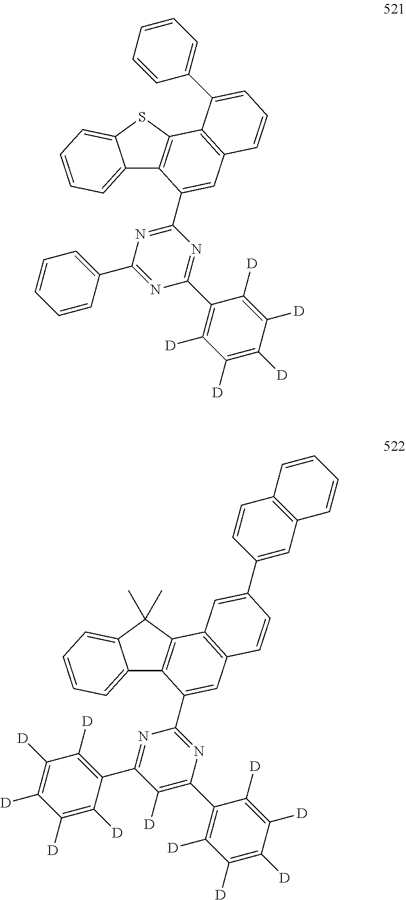 [SMILES: [2H]c1c([2H])c([2H])c(-c2nc(-c3ccccc3)nc(-c3cc4cccc(-c5ccccc5)c4c4sc5ccccc5c34)n2)c([2H])c1[2H] ; [2H]c1c(-c2c([2H])c([2H])c([2H])c([2H])c2[2H])nc(-c2cc3ccc(-c4ccc5ccccc5c4)cc3c3c2-c2ccccc2C3(C)C)nc1-c1c([2H])c([2H])c([2H])c([2H])c1[2H]]

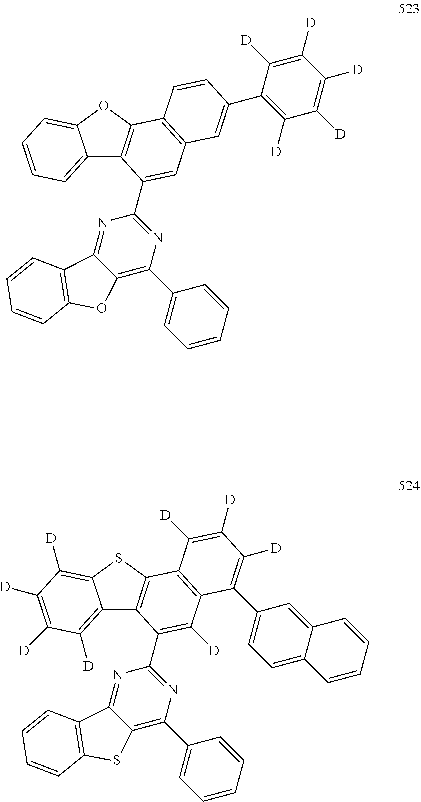 [SMILES: [2H]c1c([2H])c([2H])c(-c2ccc3c(c2)cc(-c2nc(-c4ccccc4)c4oc5ccccc5c4n2)c2c4ccccc4oc32)c([2H])c1[2H] ; [2H]c1c([2H])c([2H])c2c(sc3c4c([2H])c([2H])c([2H])c(-c5ccc6ccccc6c5)c4c([2H])c(-c4nc(-c5ccccc5)c5sc6ccccc6c5n4)c32)c1[2H]]

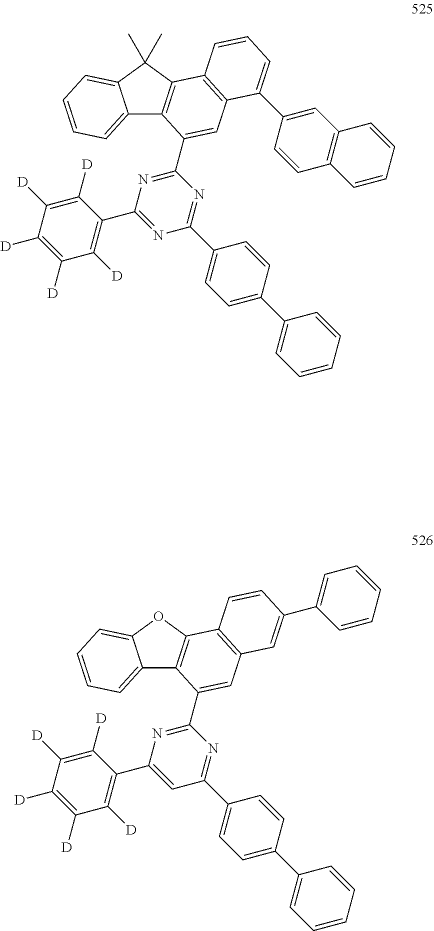 [SMILES: [2H]c1c([2H])c([2H])c(-c2nc(-c3ccc(-c4ccccc4)cc3)nc(-c3cc4c(-c5ccc6ccccc6c5)cccc4c4c3-c3ccccc3C4(C)C)n2)c([2H])c1[2H] ; [2H]c1c([2H])c([2H])c(-c2cc(-c3ccc(-c4ccccc4)cc3)nc(-c3cc4cc(-c5ccccc5)ccc4c4oc5ccccc5c34)n2)c([2H])c1[2H]]

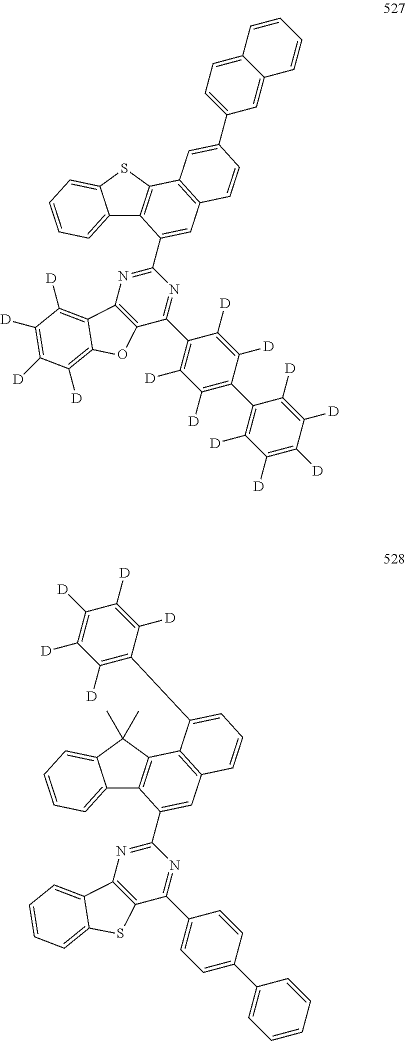 [SMILES: [2H]c1c([2H])c([2H])c(-c2c([2H])c([2H])c(-c3nc(-c4cc5ccc(-c6ccc7ccccc7c6)cc5c5sc6ccccc6c45)nc4c3oc3c([2H])c([2H])c([2H])c([2H])c34)c([2H])c2[2H])c([2H])c1[2H] ; [2H]c1c([2H])c([2H])c(-c2cccc3cc(-c4nc(-c5ccc(-c6ccccc6)cc5)c5sc6ccccc6c5n4)c4c(c23)C(C)(C)c2ccccc2-4)c([2H])c1[2H]]

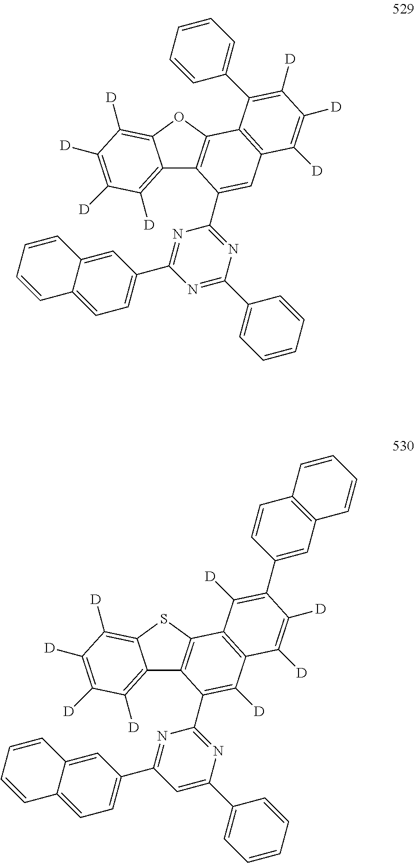 [SMILES: [2H]c1c([2H])c(-c2ccccc2)c2c(cc(-c3nc(-c4ccccc4)nc(-c4ccc5ccccc5c4)n3)c3c2oc2c([2H])c([2H])c([2H])c([2H])c23)c1[2H] ; [2H]c1c([2H])c([2H])c2c(sc3c4c([2H])c(-c5ccc6ccccc6c5)c([2H])c([2H])c4c([2H])c(-c4nc(-c5ccccc5)cc(-c5ccc6ccccc6c5)n4)c32)c1[2H]]

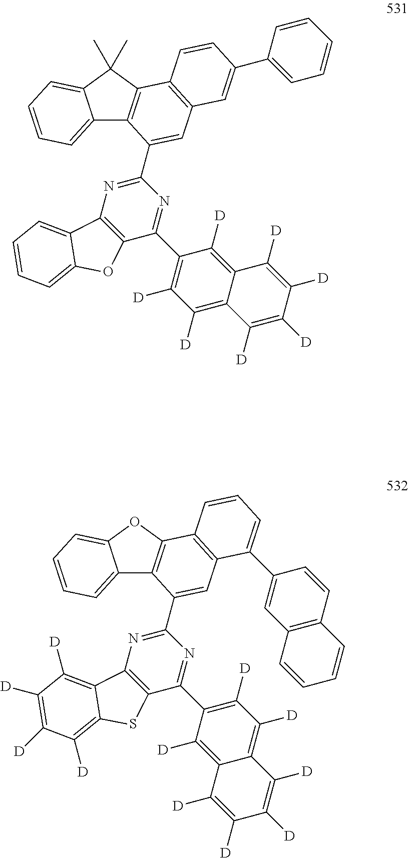 [SMILES: [2H]c1c([2H])c([2H])c2c([2H])c(-c3nc(-c4cc5cc(-c6ccccc6)ccc5c5c4-c4ccccc4C5(C)C)nc4c3oc3ccccc34)c([2H])c([2H])c2c1[2H] ; [2H]c1c([2H])c([2H])c2c(sc3c(-c4c([2H])c([2H])c5c([2H])c([2H])c([2H])c([2H])c5c4[2H])nc(-c4cc5c(-c6ccc7ccccc7c6)cccc5c5oc6ccccc6c45)nc32)c1[2H]]

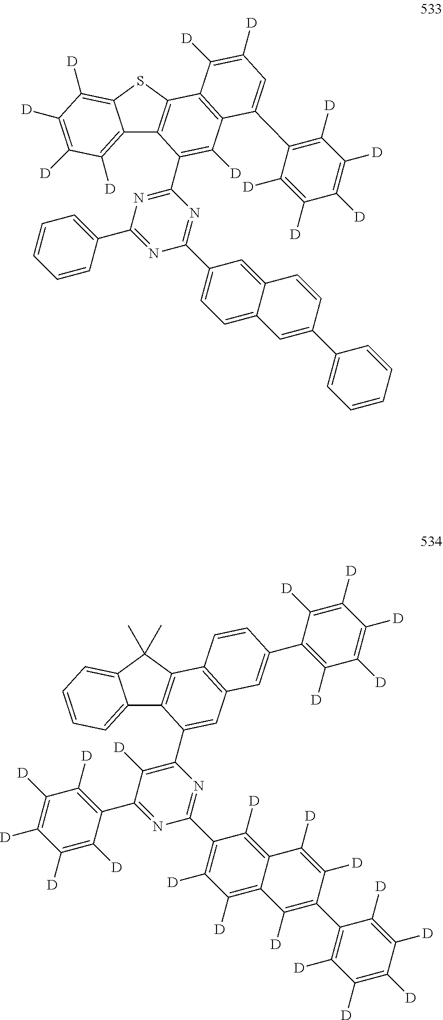 [SMILES: [2H]c1cc(-c2c([2H])c([2H])c([2H])c([2H])c2[2H])c2c([2H])c(-c3nc(-c4ccccc4)nc(-c4ccc5cc(-c6ccccc6)ccc5c4)n3)c3c(sc4c([2H])c([2H])c([2H])c([2H])c43)c2c1[2H] ; [2H]c1c(-c2cc3cc(-c4c([2H])c([2H])c([2H])c([2H])c4[2H])ccc3c3c2-c2ccccc2C3(C)C)nc(-c2c([2H])c([2H])c3c([2H])c(-c4c([2H])c([2H])c([2H])c([2H])c4[2H])c([2H])c([2H])c3c2[2H])nc1-c1c([2H])c([2H])c([2H])c([2H])c1[2H]]

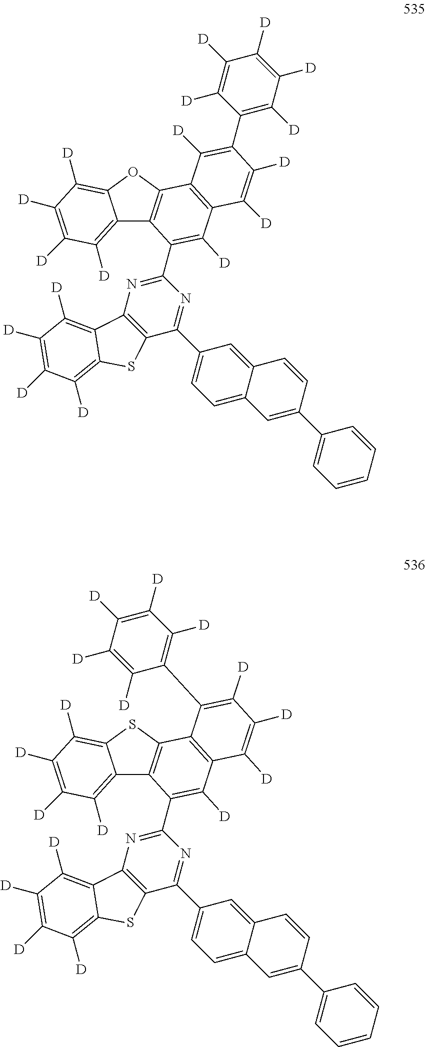 [SMILES: [2H]c1c([2H])c([2H])c(-c2c([2H])c([2H])c3c([2H])c(-c4nc(-c5ccc6cc(-c7ccccc7)ccc6c5)c5sc6c([2H])c([2H])c([2H])c([2H])c6c5n4)c4c(oc5c([2H])c([2H])c([2H])c([2H])c54)c3c2[2H])c([2H])c1[2H] ; [2H]c1c([2H])c([2H])c(-c2c([2H])c([2H])c([2H])c3c([2H])c(-c4nc(-c5ccc6cc(-c7ccccc7)ccc6c5)c5sc6c([2H])c([2H])c([2H])c([2H])c6c5n4)c4c(sc5c([2H])c([2H])c([2H])c([2H])c54)c23)c([2H])c1[2H]]

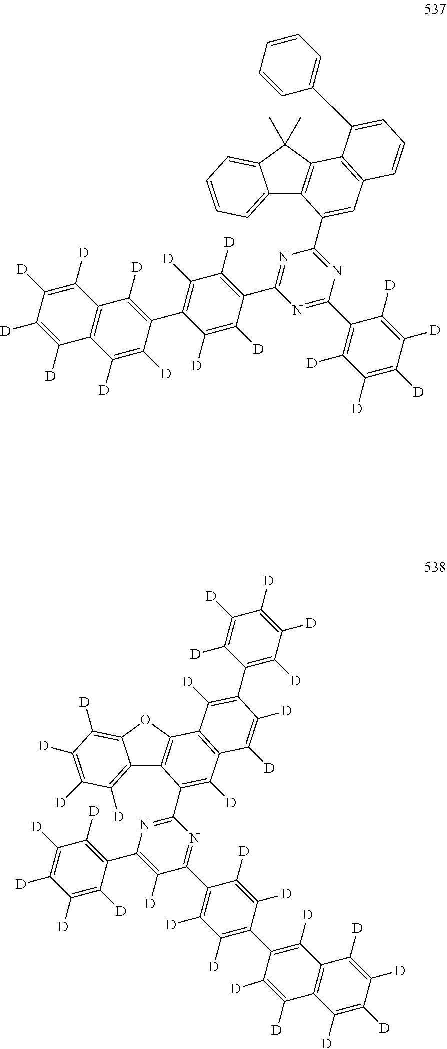 [SMILES: [2H]c1c([2H])c([2H])c(-c2nc(-c3cc4cccc(-c5ccccc5)c4c4c3-c3ccccc3C4(C)C)nc(-c3c([2H])c([2H])c(-c4c([2H])c([2H])c5c([2H])c([2H])c([2H])c([2H])c5c4[2H])c([2H])c3[2H])n2)c([2H])c1[2H] ; [2H]c1c(-c2c([2H])c([2H])c([2H])c([2H])c2[2H])nc(-c2c([2H])c3c([2H])c([2H])c(-c4c([2H])c([2H])c([2H])c([2H])c4[2H])c([2H])c3c3oc4c([2H])c([2H])c([2H])c([2H])c4c23)nc1-c1c([2H])c([2H])c(-c2c([2H])c([2H])c3c([2H])c([2H])c([2H])c([2H])c3c2[2H])c([2H])c1[2H]]

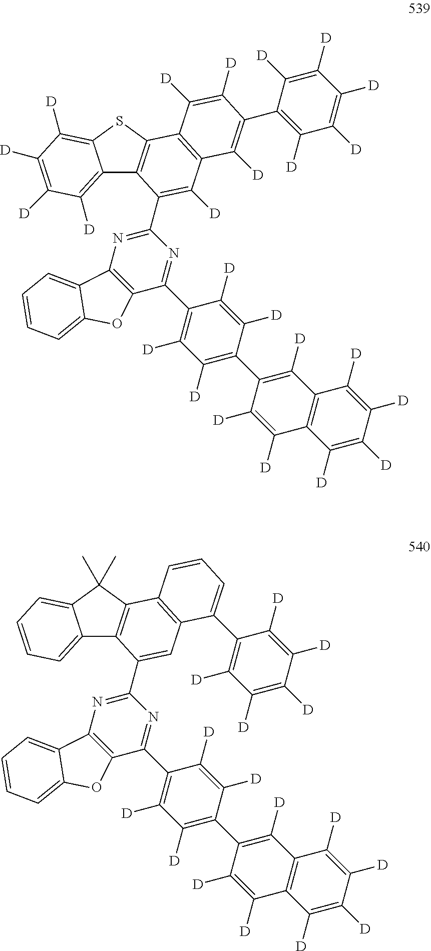 [SMILES: [2H]c1c([2H])c([2H])c(-c2c([2H])c([2H])c3c(c2[2H])c([2H])c(-c2nc(-c4c([2H])c([2H])c(-c5c([2H])c([2H])c6c([2H])c([2H])c([2H])c([2H])c6c5[2H])c([2H])c4[2H])c4oc5ccccc5c4n2)c2c3sc3c([2H])c([2H])c([2H])c([2H])c32)c([2H])c1[2H] ; [2H]c1c([2H])c([2H])c(-c2cccc3c4c(c(-c5nc(-c6c([2H])c([2H])c(-c7c([2H])c([2H])c8c([2H])c([2H])c([2H])c([2H])c8c7[2H])c([2H])c6[2H])c6oc7ccccc7c6n5)cc23)-c2ccccc2C4(C)C)c([2H])c1[2H]]

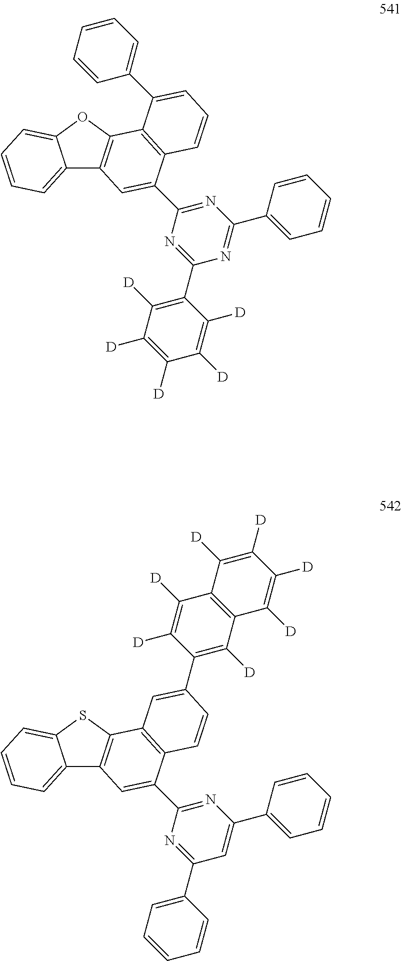 [SMILES: [2H]c1c([2H])c([2H])c(-c2nc(-c3ccccc3)nc(-c3cc4c5ccccc5oc4c4c(-c5ccccc5)cccc34)n2)c([2H])c1[2H] ; [2H]c1c([2H])c([2H])c2c([2H])c(-c3ccc4c(-c5nc(-c6ccccc6)cc(-c6ccccc6)n5)cc5c6ccccc6sc5c4c3)c([2H])c([2H])c2c1[2H]]

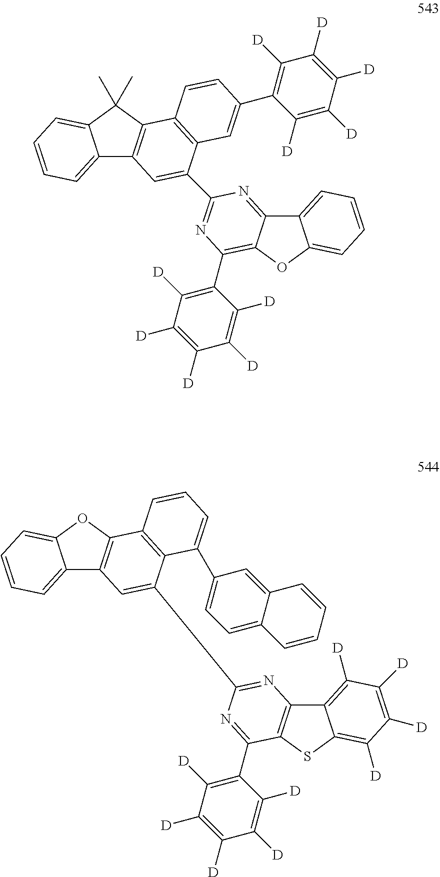 [SMILES: [2H]c1c([2H])c([2H])c(-c2ccc3c4c(cc(-c5nc(-c6c([2H])c([2H])c([2H])c([2H])c6[2H])c6oc7ccccc7c6n5)c3c2)-c2ccccc2C4(C)C)c([2H])c1[2H] ; [2H]c1c([2H])c([2H])c(-c2nc(-c3cc4c5ccccc5oc4c4cccc(-c5ccc6ccccc6c5)c34)nc3c2sc2c([2H])c([2H])c([2H])c([2H])c23)c([2H])c1[2H]]

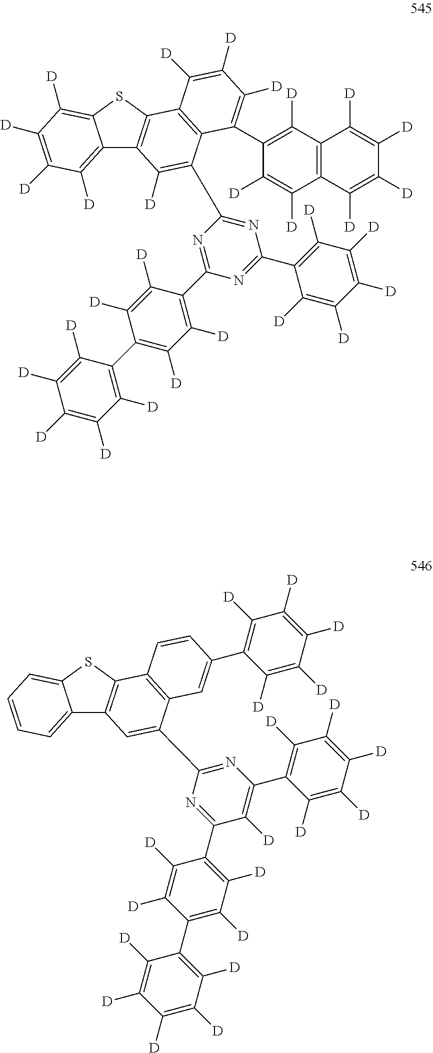 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c4c(sc5c([2H])c([2H])c([2H])c([2H])c54)c4c([2H])c([2H])c([2H])c(-c5c([2H])c([2H])c6c([2H])c([2H])c([2H])c([2H])c6c5[2H])c34)n2)c([2H])c1[2H] ; [2H]c1c(-c2c([2H])c([2H])c([2H])c([2H])c2[2H])nc(-c2cc3c4ccccc4sc3c3ccc(-c4c([2H])c([2H])c([2H])c([2H])c4[2H])cc23)nc1-c1c([2H])c([2H])c(-c2c([2H])c([2H])c([2H])c([2H])c2[2H])c([2H])c1[2H]]

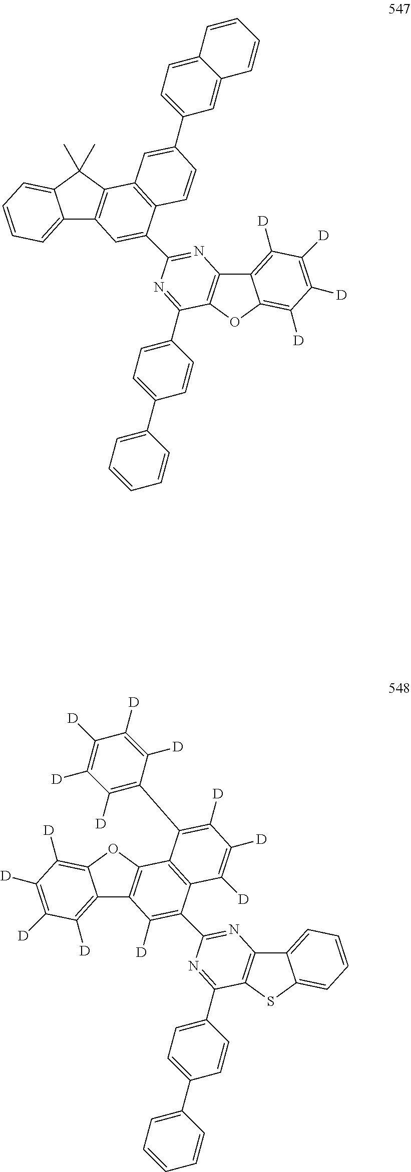 [SMILES: [2H]c1c([2H])c([2H])c2c(oc3c(-c4ccc(-c5ccccc5)cc4)nc(-c4cc5c(c6cc(-c7ccc8ccccc8c7)ccc46)C(C)(C)c4ccccc4-5)nc32)c1[2H] ; [2H]c1c([2H])c([2H])c(-c2c([2H])c([2H])c([2H])c3c(-c4nc(-c5ccc(-c6ccccc6)cc5)c5sc6ccccc6c5n4)c([2H])c4c(oc5c([2H])c([2H])c([2H])c([2H])c54)c23)c([2H])c1[2H]]

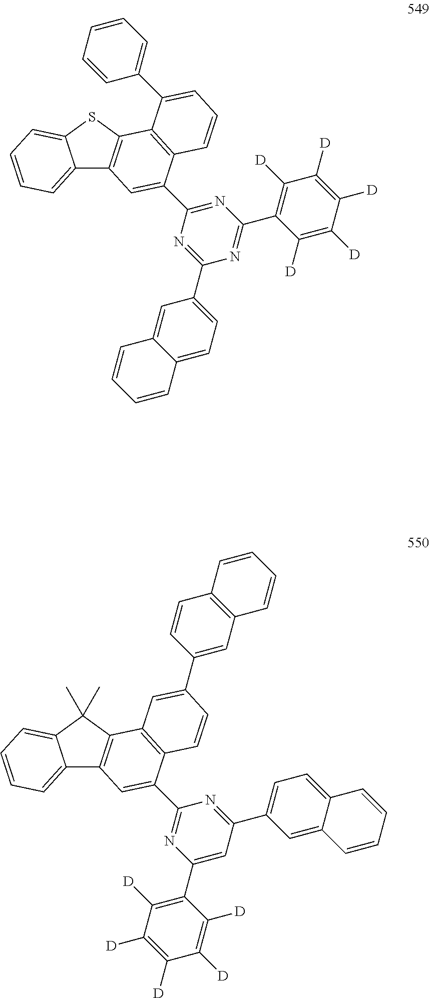 [SMILES: [2H]c1c([2H])c([2H])c(-c2nc(-c3ccc4ccccc4c3)nc(-c3cc4c5ccccc5sc4c4c(-c5ccccc5)cccc34)n2)c([2H])c1[2H] ; [2H]c1c([2H])c([2H])c(-c2cc(-c3ccc4ccccc4c3)nc(-c3cc4c(c5cc(-c6ccc7ccccc7c6)ccc35)C(C)(C)c3ccccc3-4)n2)c([2H])c1[2H]]

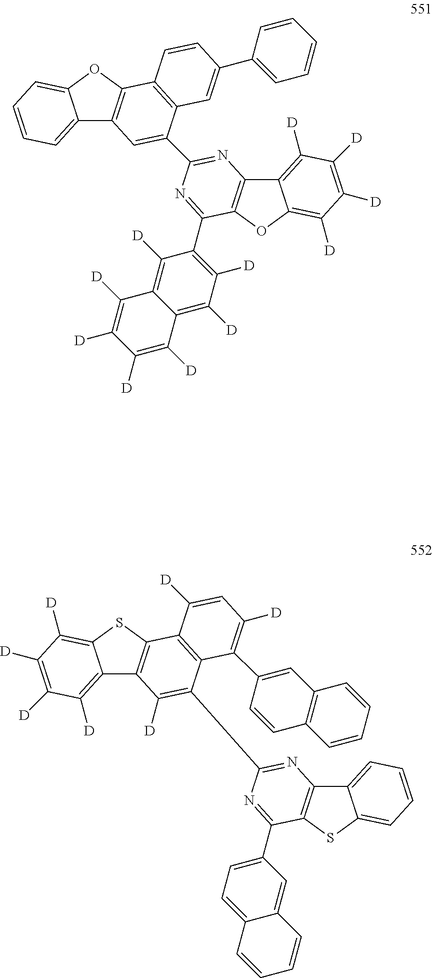 [SMILES: [2H]c1c([2H])c([2H])c2c(oc3c(-c4c([2H])c([2H])c5c([2H])c([2H])c([2H])c([2H])c5c4[2H])nc(-c4cc5c6ccccc6oc5c5ccc(-c6ccccc6)cc45)nc32)c1[2H] ; [2H]c1cc([2H])c2c(c1-c1ccc3ccccc3c1)c(-c1nc(-c3ccc4ccccc4c3)c3sc4ccccc4c3n1)c([2H])c1c2sc2c([2H])c([2H])c([2H])c([2H])c21]

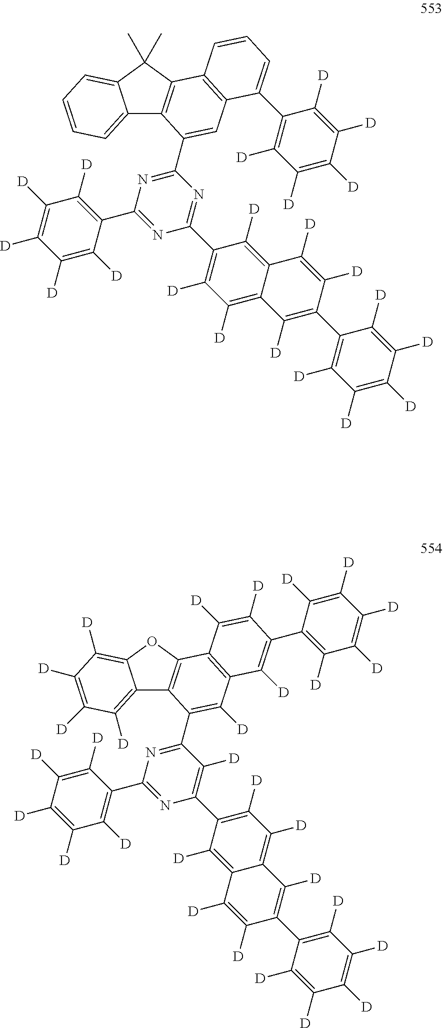 [SMILES: [2H]c1c([2H])c([2H])c(-c2nc(-c3cc4c(-c5c([2H])c([2H])c([2H])c([2H])c5[2H])cccc4c4c3-c3ccccc3C4(C)C)nc(-c3c([2H])c([2H])c4c([2H])c(-c5c([2H])c([2H])c([2H])c([2H])c5[2H])c([2H])c([2H])c4c3[2H])n2)c([2H])c1[2H] ; [2H]c1c(-c2c([2H])c([2H])c3c([2H])c(-c4c([2H])c([2H])c([2H])c([2H])c4[2H])c([2H])c([2H])c3c2[2H])nc(-c2c([2H])c([2H])c([2H])c([2H])c2[2H])nc1-c1c([2H])c2c([2H])c(-c3c([2H])c([2H])c([2H])c([2H])c3[2H])c([2H])c([2H])c2c2oc3c([2H])c([2H])c([2H])c([2H])c3c12]

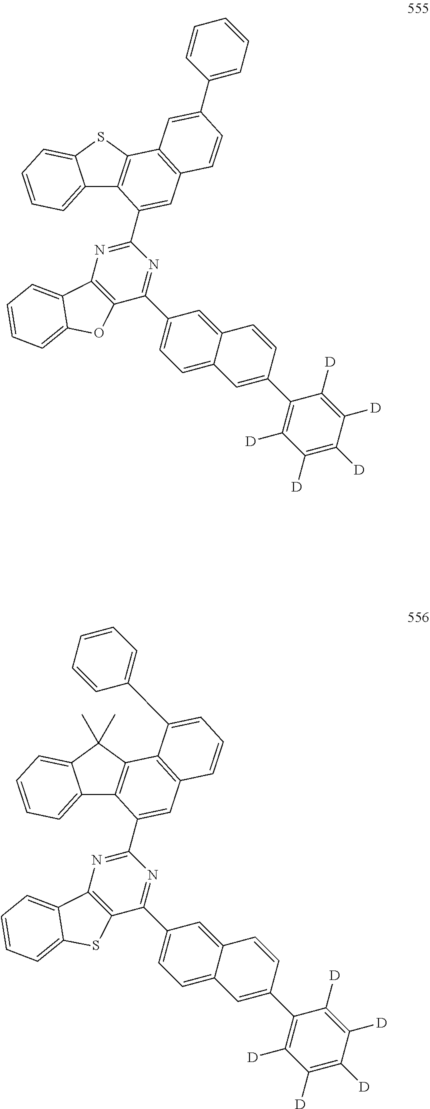 [SMILES: [2H]c1c([2H])c([2H])c(-c2ccc3cc(-c4nc(-c5cc6ccc(-c7ccccc7)cc6c6sc7ccccc7c56)nc5c4oc4ccccc45)ccc3c2)c([2H])c1[2H] ; [2H]c1c([2H])c([2H])c(-c2ccc3cc(-c4nc(-c5cc6cccc(-c7ccccc7)c6c6c5-c5ccccc5C6(C)C)nc5c4sc4ccccc45)ccc3c2)c([2H])c1[2H]]

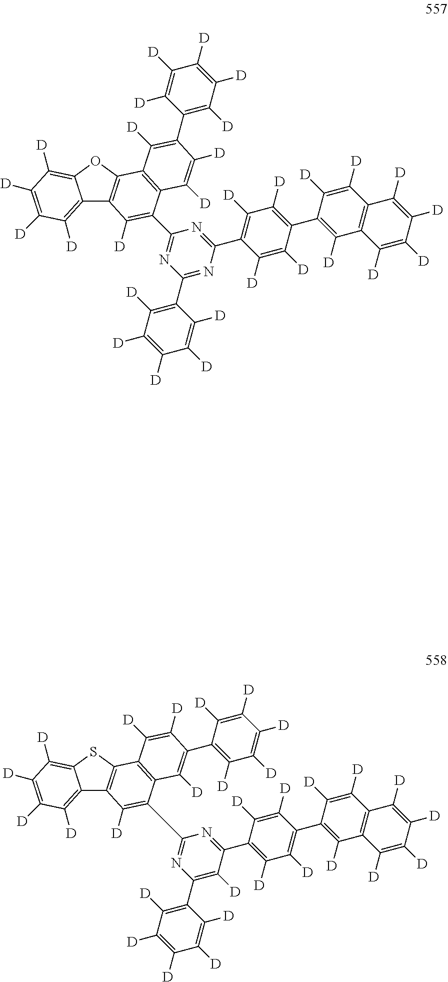

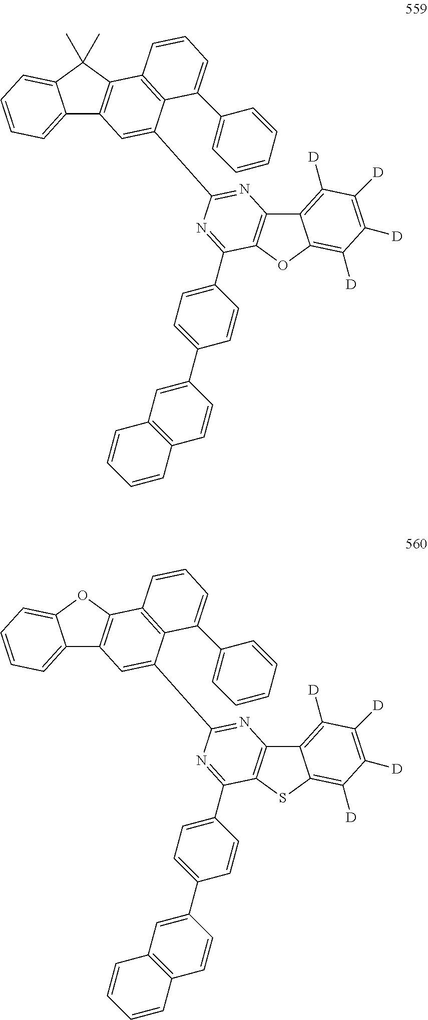 [SMILES: [2H]c1c([2H])c([2H])c2c(oc3c(-c4ccc(-c5ccc6ccccc6c5)cc4)nc(-c4cc5c(c6cccc(-c7ccccc7)c46)C(C)(C)c4ccccc4-5)nc32)c1[2H] ; [2H]c1c([2H])c([2H])c2c(sc3c(-c4ccc(-c5ccc6ccccc6c5)cc4)nc(-c4cc5c6ccccc6oc5c5cccc(-c6ccccc6)c45)nc32)c1[2H]]

In an exemplary embodiment of the present application, the compound is just one example and is not limited thereto, and may include other compounds included in Chemical Formula 1 which includes an additional substituent. In addition, the substitution position of deuterium of the compound may be present while a specific position is excluded and hydrogen and deuterium are mixed during the process of deuterium substitution and synthesis.

Further, various substituents may be introduced into the structure of Chemical Formula 1 to synthesize a compound having inherent characteristics of a substituent introduced. For example, a substituent usually used for a hole injection material, a hole transport material, a light emitting material, an electron transport material and an electron injection material, which are used when manufacturing an organic light emitting device, may be introduced into the core structure to synthesize a material which satisfies conditions required for each organic material layer.

In addition, by introducing various substituents into the structure of Chemical Formula 1 or changing the binding position, the bandgap may be finely adjusted, and meanwhile, the characteristics at the interface between the organic material layers may be improved.

In addition, the compound of Chemical Formula 1 has excellent thermal stability, and such thermal stability provides driving stability to the organic light emitting device and improves service life characteristics.

In an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In another exemplary embodiment, provided is an organic light emitting device including: a first electrode; a second electrode provided to face the first electrode; and an organic material layer having one or more layers provided between the first electrode and the second electrode, in which one or more layers of the organic material layer include one heterocyclic compound represented by Chemical Formula 1.

In still another exemplary embodiment, provided is an organic light emitting device including: a first electrode; a second electrode provided to face the first electrode; and an organic material layer having one or more layers provided between the first electrode and the second electrode, in which one or more layers of the organic material layer include two or more of the heterocyclic compound represented by Chemical Formula 1.

In yet another exemplary embodiment, the heterocyclic compound represented by Chemical Formula 1 can be used as a light emitting material fora light emitting layer of the organic light emitting device.

In still yet another exemplary embodiment, the heterocyclic compound represented by Chemical Formula 1 can be used as a host material for a light emitting layer of the organic light emitting device.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device.

In an exemplary embodiment of the present application, the organic light emitting device may be a green organic light emitting device, and the heterocyclic compound represented by Chemical Formula 1 may be used as a material for the green organic light emitting device.

In an exemplary embodiment of the present application, the organic light emitting device may be a red organic light emitting device, and the heterocyclic compound represented by Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for a light emitting layer of the blue organic light emitting device.

In an exemplary embodiment of the present application, the organic light emitting device may be a green organic light emitting device, and the heterocyclic compound represented by Chemical Formula 1 may be used as a material for a light emitting layer of the green organic light emitting device.

In an exemplary embodiment of the present application, the organic light emitting device may be a red organic light emitting device, and the heterocyclic compound represented by Chemical Formula 1 may be used as a material for a light emitting layer of the red organic light emitting device.

In an exemplary embodiment of the present application, the specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃, which is a green phosphorescent dopant, may be used.

In an exemplary embodiment of the present application, as the iridium-based dopant, (piq)₂(Ir)(acac), which is a red phosphorescent dopant, may be used.

In an exemplary embodiment of the present application, provided is an organic light emitting device, in which the organic material layer of the organic light emitting device includes a light emitting layer, and the light emitting layer includes the heterocyclic compound.

In an exemplary embodiment of the present application, provided is an organic light emitting device, in which the organic material layer of the organic light emitting device includes a light emitting layer, and the light emitting layer includes a host material, and the host material includes the heterocyclic compound.

In the organic light emitting device of the present invention, the organic material layer includes an electron injection layer or an electron transport layer, and the electron injection layer or electron transport layer may include the heterocyclic compound.

In another organic light emitting device, the organic material layer includes a hole blocking layer, and the hole blocking layer may include the heterocyclic compound.

In still another organic light emitting device, the organic material layer includes an electron blocking layer, and the electron blocking layer may include the heterocyclic compound.

In yet another organic light emitting device, the organic material layer includes a hole transport layer, a light emitting layer or an electron blocking layer, and the hole transport layer, the light emitting layer or the electron blocking layer may include the heterocyclic compound.

In still yet another organic light emitting device, the organic material layer includes a hole transport layer or a hole transport auxiliary layer, and the hole transport layer or the hole transport auxiliary layer may include the heterocyclic compound.

In the organic light emitting device of the present application, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PE DOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-M TDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

A s an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

The organic light emitting device of the present invention may further include one or two or more layers selected from the group consisting of a light emitting layer, a hole injection layer, a hole transport layer, a hole transport auxiliary layer, an electron blocking layer, a hole blocking layer, an electron transport layer and an electron injection layer.

The organic light emitting device of the present invention may further include one or two or more layers selected from the group consisting of a light emitting layer, a hole injection layer, a hole transport layer, an electron blocking layer, a hole blocking layer, an electron transport layer and an electron injection layer.

FIGS. 1 to 4 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

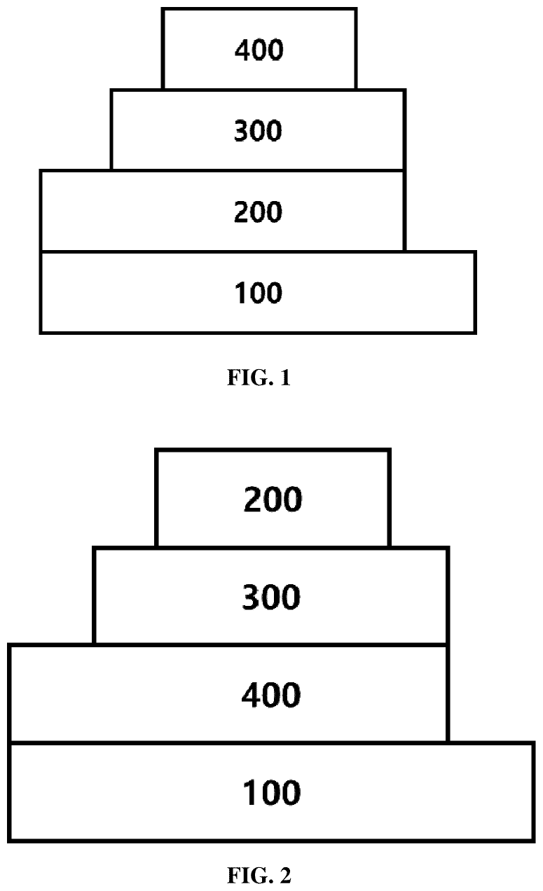

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

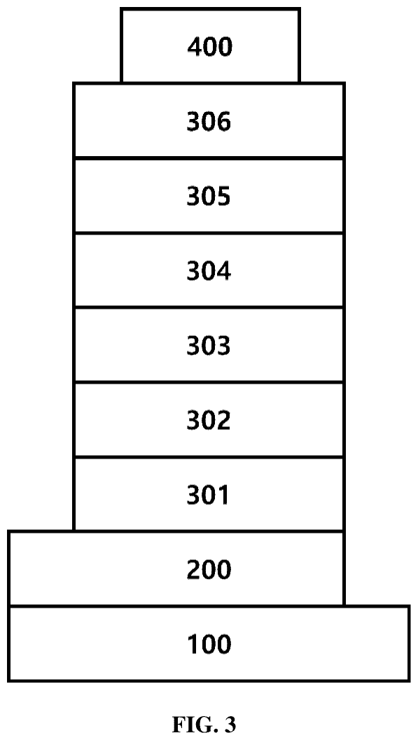

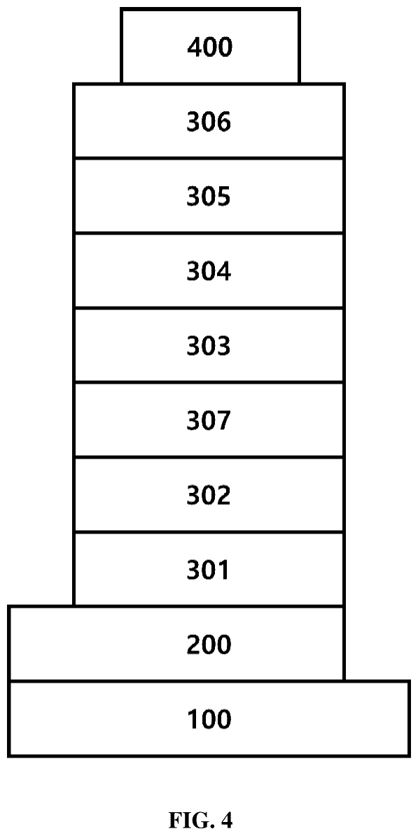

FIGS. 3 and 4 exemplify a case where an organic material layer is a multilayer.

An organic light emitting device according to FIG. 3 includes a hole injection layer 301, a hole transport layer 302, a light emitting layer 303, a hole blocking layer 304, an electron transport layer 305, and an electron injection layer 306.

An organic light emitting device according to FIG. 4 includes a hole injection layer 301, a hole transport layer 302, an electron blocking layer 307, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In an exemplary embodiment of the present application, provided is an organic light emitting device in which the organic material layer of the organic light emitting device including the heterocyclic compound represented by Chemical Formula 1 further includes a heterocyclic compound represented by the following Chemical Formula 2.

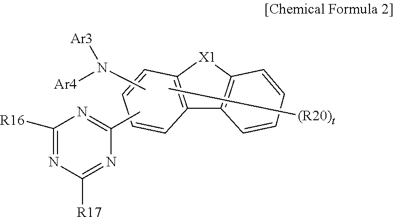

In Chemical Formula 2,
X1 is O or S,
R20 is hydrogen; deuterium; a halogen; a cyano group; a substituted or unsubstituted C1 to C60 alkyl group; a substituted or unsubstituted C6 to C60 aryl group; or a substituted or unsubstituted C2 to C60 heteroaryl group,
t is an integer from 0 to 6, and when t is 2 or higher, substituents in the parenthesis are the same as or different from each other,
Ar3 and Ar4 are the same as or different from each other, and are each independently a substituted or unsubstituted C6 to C60 aryl group; or a substituted or unsubstituted C2 to C60 heteroaryl group, and
R16 and R17 are the same as or different from each other, and are each independently a substituted or unsubstituted C6 to C60 aryl group; or a substituted or unsubstituted C2 to C60 heteroaryl group.

In an exemplary embodiment of the present application, X1 is 0.

In an exemplary embodiment of the present application, R20 may be hydrogen; deuterium; a halogen; a cyano group; a substituted or unsubstituted C1 to C40 alkyl group; a substituted or unsubstituted C6 to C40 aryl group; or a substituted or unsubstituted C2 to C40 heteroaryl group.

In another exemplary embodiment, R20 may be hydrogen; deuterium; a halogen; a cyano group; a substituted or unsubstituted C1 to C20 alkyl group; a substituted or unsubstituted C6 to C20 aryl group; or a substituted or unsubstituted C2 to C20 heteroaryl group.

In still another exemplary embodiment, R20 may be hydrogen; deuterium; a substituted or unsubstituted C6 to C20 aryl group; or a substituted or unsubstituted C2 to C20 heteroaryl group.

In yet another exemplary embodiment, R20 may be hydrogen; or deuterium.

In an exemplary embodiment of the present application, the deuterium content of Chemical Formula 2 may be 0% to 100%.

In another exemplary embodiment, the deuterium content of Chemical Formula 2 may be 0%, or 20% to 100%.

In still another exemplary embodiment, the deuterium content of Chemical Formula 2 may be 0%, or 40% to 100%.

In yet another exemplary embodiment, the deuterium content of Chemical Formula 2 may be 0%, or 60% to 100%.

In still yet another exemplary embodiment, the deuterium content of Chemical Formula 2 may be 0%, or 80% to 100%.

In a further exemplary embodiment, the deuterium content of Chemical Formula 2 may be 0%, or 100%.

In an exemplary embodiment of the present application, Ar3 and Ar4 are the same as or different from each other, and may be each independently a substituted or unsubstituted C6 to C40 aryl group; or a substituted or unsubstituted C2 to C40 heteroaryl group.

In another exemplary embodiment, Ar3 and Ar4 are the same as or different from each other, and may be each independently a substituted or unsubstituted C6 to C20 aryl group; or a substituted or unsubstituted C2 to C20 heteroaryl group.

In still another exemplary embodiment, Ar3 and Ar4 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted triphenylene group; a substituted or unsubstituted dibenzofuran group; a substituted or unsubstituted dibenzothiophene group; or a substituted or unsubstituted carbazole group.

In yet another exemplary embodiment, Ar3 and Ar4 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted dibenzofuran group; or a substituted or unsubstituted dibenzothiophene group.

In still yet another exemplary embodiment, Ar3 and Ar4 are the same as or different from each other, and may be each independently a phenyl group which is unsubstituted or substituted with deuterium or a C6 to C40 aryl group; a biphenyl group which is unsubstituted or substituted with deuterium or a C6 to C40 aryl group; a terphenyl group which is unsubstituted or substituted with deuterium or a C6 to C40 aryl group; a naphthyl group which is unsubstituted or substituted with deuterium or a C6 to C40 aryl group; a substituted or unsubstituted phenanthrene group; a substituted or unsubstituted dibenzofuran group; or a substituted or unsubstituted dibenzothiophene group.

In a further exemplary embodiment, Ar3 and Ar4 are the same as or different from each other, and may be each independently a phenyl group which is unsubstituted or substituted with deuterium, a phenyl group substituted with deuterium or a naphthyl group; a biphenyl group which is unsubstituted or substituted with deuterium; a terphenyl group which is unsubstituted or substituted with deuterium; a naphthyl group which is unsubstituted or substituted with deuterium or a phenyl group; a phenanthrene group; a dibenzofuran group; or a dibenzothiophene group.

In an exemplary embodiment of the present application, R16 and R17 are the same as or different from each other, and may be each independently a substituted or unsubstituted C6 to C40 aryl group; or a substituted or unsubstituted C2 to C40 heteroaryl group.

In another exemplary embodiment, R16 and R17 are the same as or different from each other, and may be each independently a substituted or unsubstituted C6 to C20 aryl group; or a substituted or unsubstituted C2 to C20 heteroaryl group.

In still another exemplary embodiment, R16 and R17 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted triphenylene group; a substituted or unsubstituted dibenzofuran group; a substituted or unsubstituted dibenzothiophene group; or a substituted or unsubstituted carbazole group.

In yet another exemplary embodiment, R16 and R17 are the same as or different from each other, and may be each independently a phenyl group which is unsubstituted or substituted with deuterium; a biphenyl group which is unsubstituted or substituted with deuterium; a terphenyl group which is unsubstituted or substituted with deuterium; a naphthyl group which is unsubstituted or substituted with deuterium; a phenanthrene group which is unsubstituted or substituted with deuterium; a triphenylene group which is unsubstituted or substituted with deuterium; a substituted or unsubstituted dibenzofuran group; a substituted or unsubstituted dibenzothiophene group; or a substituted or unsubstituted carbazole group.

In still yet another exemplary embodiment, R16 and R17 are the same as or different from each other, and may be each independently a phenyl group which is unsubstituted or substituted with deuterium; a biphenyl group; a terphenyl group; a naphthyl group; a phenanthrene group; a triphenylene group; a dibenzofuran group; a dibenzothiophene group; or a carbazole group.

In a further exemplary embodiment, R16 and R17 are the same as or different from each other, and may be each independently a phenyl group which is unsubstituted or substituted with deuterium; a naphthyl group; a dibenzofuran group; or a dibenzothiophene group.

In an exemplary embodiment of the present invention, provided is an organic light emitting device in which Chemical Formula 2 is represented by any one of the following compounds.

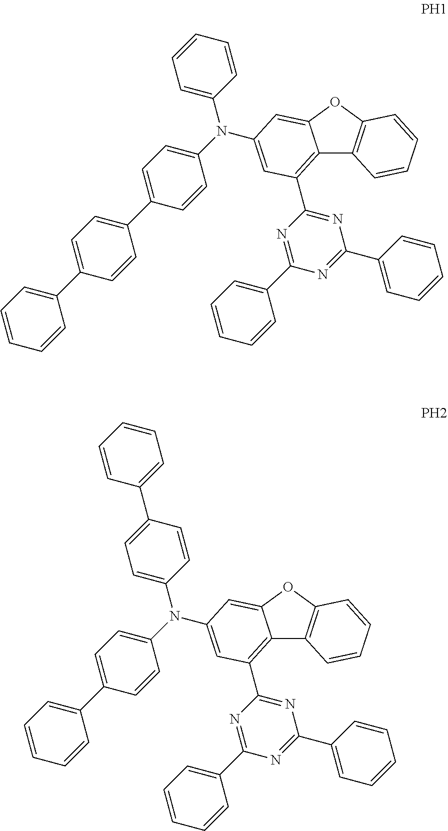 [SMILES: c1ccc(-c2ccc(-c3ccc(N(c4ccccc4)c4cc(-c5nc(-c6ccccc6)nc(-c6ccccc6)n5)c5c(c4)oc4ccccc45)cc3)cc2)cc1 ; c1ccc(-c2ccc(N(c3ccc(-c4ccccc4)cc3)c3cc(-c4nc(-c5ccccc5)nc(-c5ccccc5)n4)c4c(c3)oc3ccccc34)cc2)cc1]

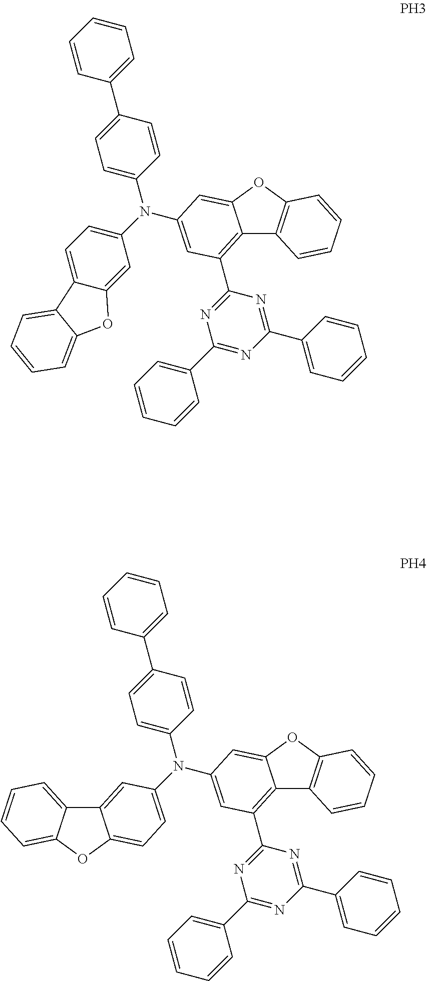 [SMILES: c1ccc(-c2ccc(N(c3ccc4c(c3)oc3ccccc34)c3cc(-c4nc(-c5ccccc5)nc(-c5ccccc5)n4)c4c(c3)oc3ccccc34)cc2)cc1 ; c1ccc(-c2ccc(N(c3cc(-c4nc(-c5ccccc5)nc(-c5ccccc5)n4)c4c(c3)oc3ccccc34)c3ccc4oc5ccccc5c4c3)cc2)cc1]

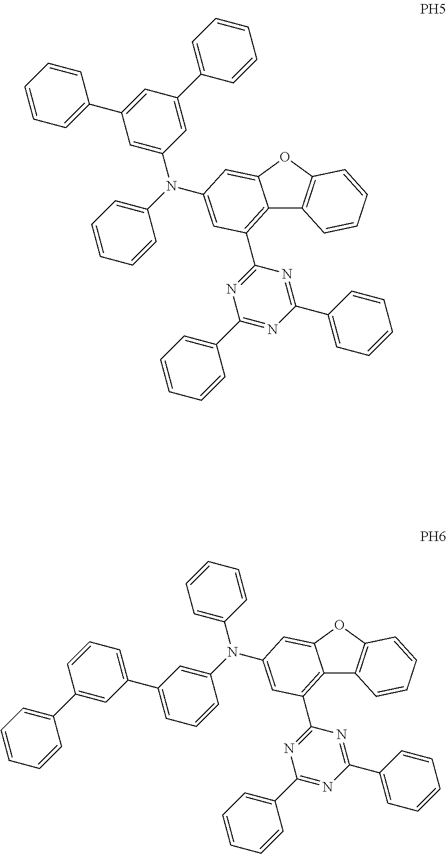 [SMILES: c1ccc(-c2cc(-c3ccccc3)cc(N(c3ccccc3)c3cc(-c4nc(-c5ccccc5)nc(-c5ccccc5)n4)c4c(c3)oc3ccccc34)c2)cc1 ; c1ccc(-c2cccc(-c3cccc(N(c4ccccc4)c4cc(-c5nc(-c6ccccc6)nc(-c6ccccc6)n5)c5c(c4)oc4ccccc45)c3)c2)cc1]

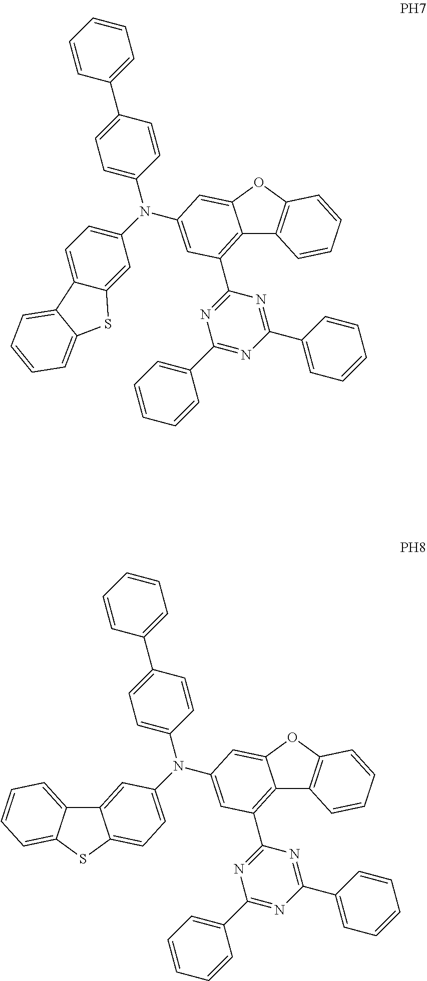 [SMILES: c1ccc(-c2ccc(N(c3cc(-c4nc(-c5ccccc5)nc(-c5ccccc5)n4)c4c(c3)oc3ccccc34)c3ccc4c(c3)sc3ccccc34)cc2)cc1 ; c1ccc(-c2ccc(N(c3cc(-c4nc(-c5ccccc5)nc(-c5ccccc5)n4)c4c(c3)oc3ccccc34)c3ccc4sc5ccccc5c4c3)cc2)cc1]

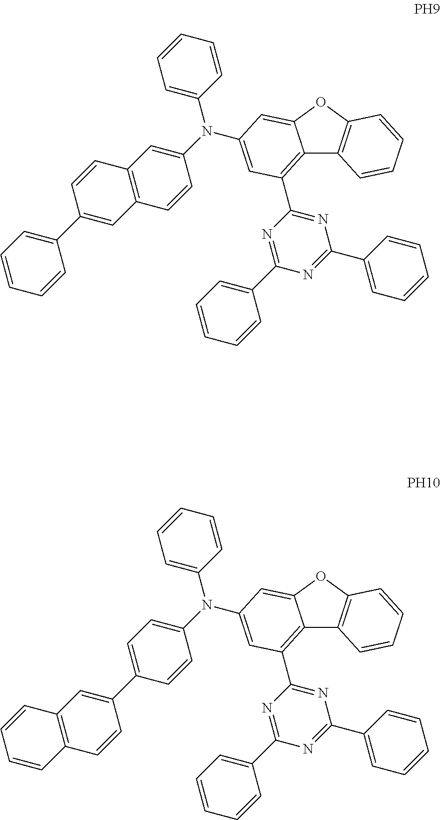 [SMILES: c1ccc(-c2ccc3cc(N(c4ccccc4)c4cc(-c5nc(-c6ccccc6)nc(-c6ccccc6)n5)c5c(c4)oc4ccccc45)ccc3c2)cc1 ; c1ccc(-c2nc(-c3ccccc3)nc(-c3cc(N(c4ccccc4)c4ccc(-c5ccc6ccccc6c5)cc4)cc4oc5ccccc5c34)n2)cc1]

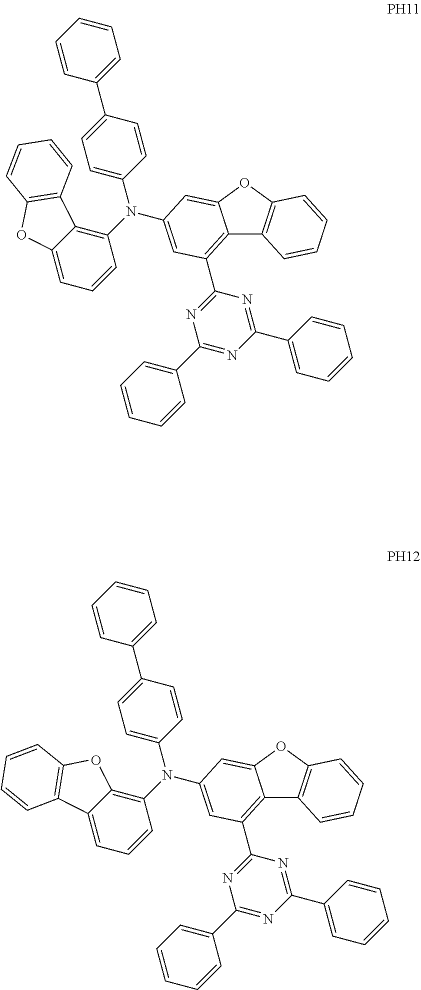 [SMILES: c1ccc(-c2ccc(N(c3cc(-c4nc(-c5ccccc5)nc(-c5ccccc5)n4)c4c(c3)oc3ccccc34)c3cccc4oc5ccccc5c34)cc2)cc1 ; c1ccc(-c2ccc(N(c3cc(-c4nc(-c5ccccc5)nc(-c5ccccc5)n4)c4c(c3)oc3ccccc34)c3cccc4c3oc3ccccc34)cc2)cc1]

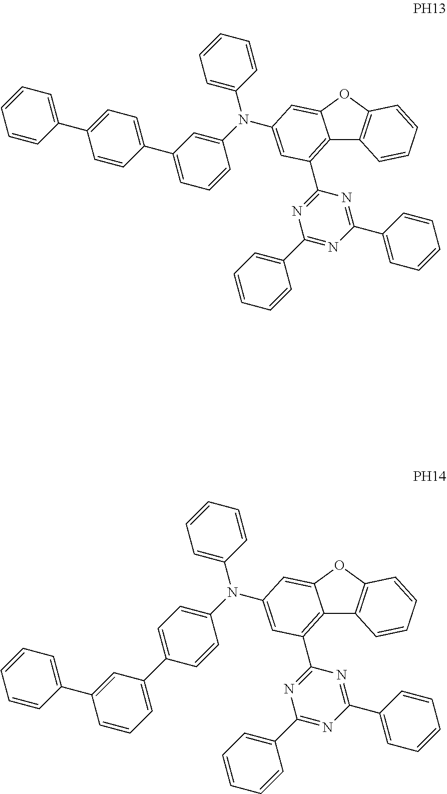 [SMILES: c1ccc(-c2ccc(-c3cccc(N(c4ccccc4)c4cc(-c5nc(-c6ccccc6)nc(-c6ccccc6)n5)c5c(c4)oc4ccccc45)c3)cc2)cc1 ; c1ccc(-c2cccc(-c3ccc(N(c4ccccc4)c4cc(-c5nc(-c6ccccc6)nc(-c6ccccc6)n5)c5c(c4)oc4ccccc45)cc3)c2)cc1]

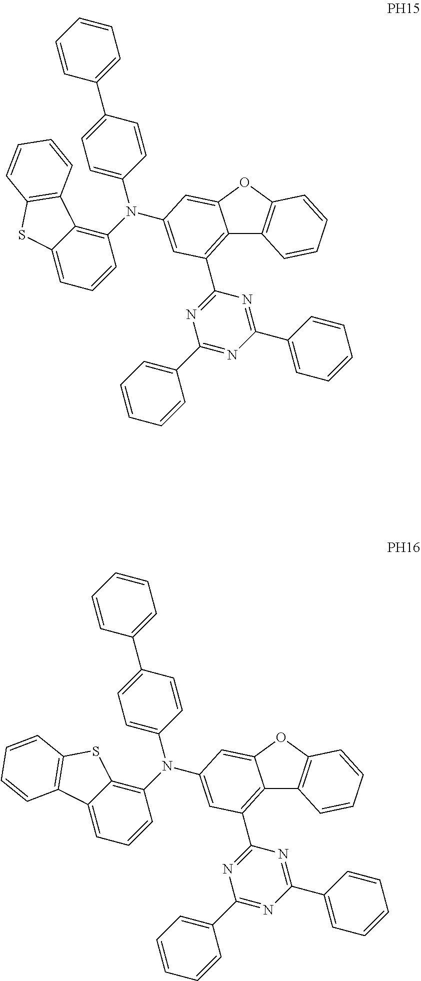 [SMILES: c1ccc(-c2ccc(N(c3cc(-c4nc(-c5ccccc5)nc(-c5ccccc5)n4)c4c(c3)oc3ccccc34)c3cccc4sc5ccccc5c34)cc2)cc1 ; c1ccc(-c2ccc(N(c3cc(-c4nc(-c5ccccc5)nc(-c5ccccc5)n4)c4c(c3)oc3ccccc34)c3cccc4c3sc3ccccc34)cc2)cc1]

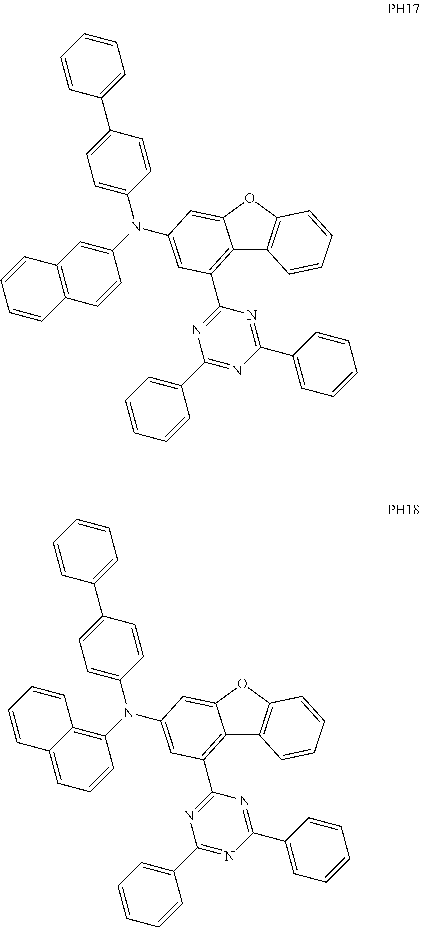 [SMILES: c1ccc(-c2ccc(N(c3ccc4ccccc4c3)c3cc(-c4nc(-c5ccccc5)nc(-c5ccccc5)n4)c4c(c3)oc3ccccc34)cc2)cc1 ; c1ccc(-c2ccc(N(c3cc(-c4nc(-c5ccccc5)nc(-c5ccccc5)n4)c4c(c3)oc3ccccc34)c3cccc4ccccc34)cc2)cc1]

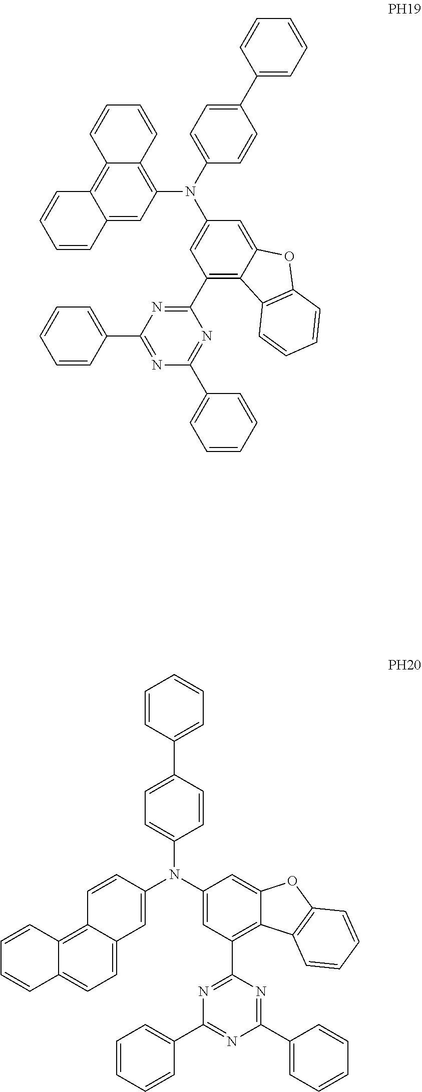 [SMILES: c1ccc(-c2ccc(N(c3cc(-c4nc(-c5ccccc5)nc(-c5ccccc5)n4)c4c(c3)oc3ccccc34)c3cc4ccccc4c4ccccc34)cc2)cc1 ; c1ccc(-c2ccc(N(c3ccc4c(ccc5ccccc54)c3)c3cc(-c4nc(-c5ccccc5)nc(-c5ccccc5)n4)c4c(c3)oc3ccccc34)cc2)cc1]

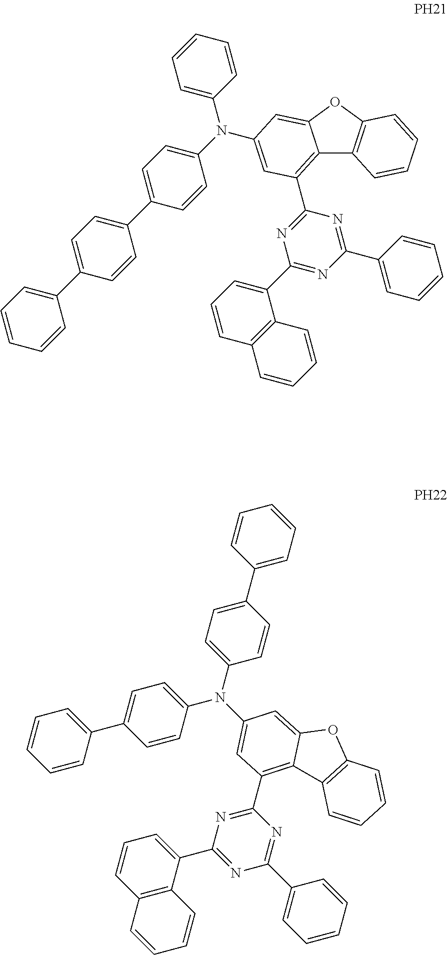 [SMILES: c1ccc(-c2ccc(-c3ccc(N(c4ccccc4)c4cc(-c5nc(-c6ccccc6)nc(-c6cccc7ccccc67)n5)c5c(c4)oc4ccccc45)cc3)cc2)cc1 ; c1ccc(-c2ccc(N(c3ccc(-c4ccccc4)cc3)c3cc(-c4nc(-c5ccccc5)nc(-c5cccc6ccccc56)n4)c4c(c3)oc3ccccc34)cc2)cc1]

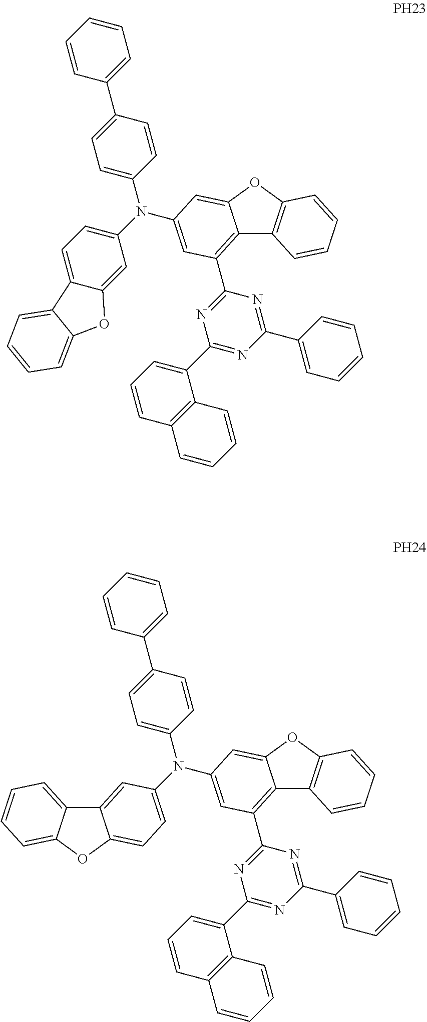 [SMILES: c1ccc(-c2ccc(N(c3ccc4c(c3)oc3ccccc34)c3cc(-c4nc(-c5ccccc5)nc(-c5cccc6ccccc56)n4)c4c(c3)oc3ccccc34)cc2)cc1 ; c1ccc(-c2ccc(N(c3cc(-c4nc(-c5ccccc5)nc(-c5cccc6ccccc56)n4)c4c(c3)oc3ccccc34)c3ccc4oc5ccccc5c4c3)cc2)cc1]

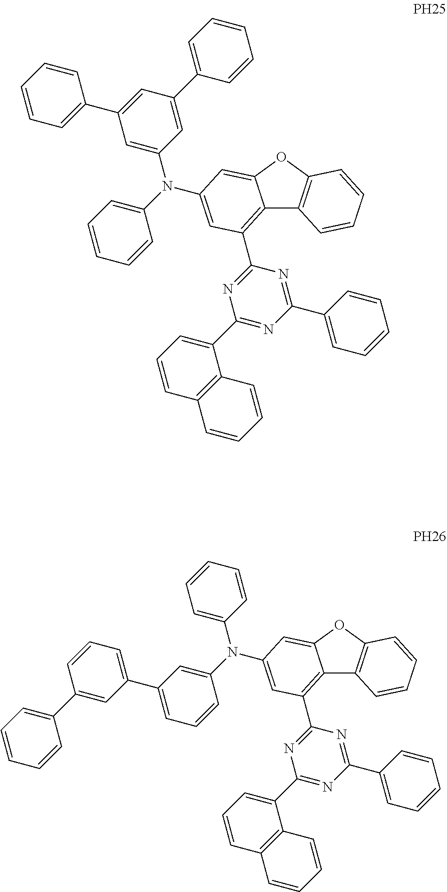 [SMILES: c1ccc(-c2cc(-c3ccccc3)cc(N(c3ccccc3)c3cc(-c4nc(-c5ccccc5)nc(-c5cccc6ccccc56)n4)c4c(c3)oc3ccccc34)c2)cc1 ; c1ccc(-c2cccc(-c3cccc(N(c4ccccc4)c4cc(-c5nc(-c6ccccc6)nc(-c6cccc7ccccc67)n5)c5c(c4)oc4ccccc45)c3)c2)cc1]

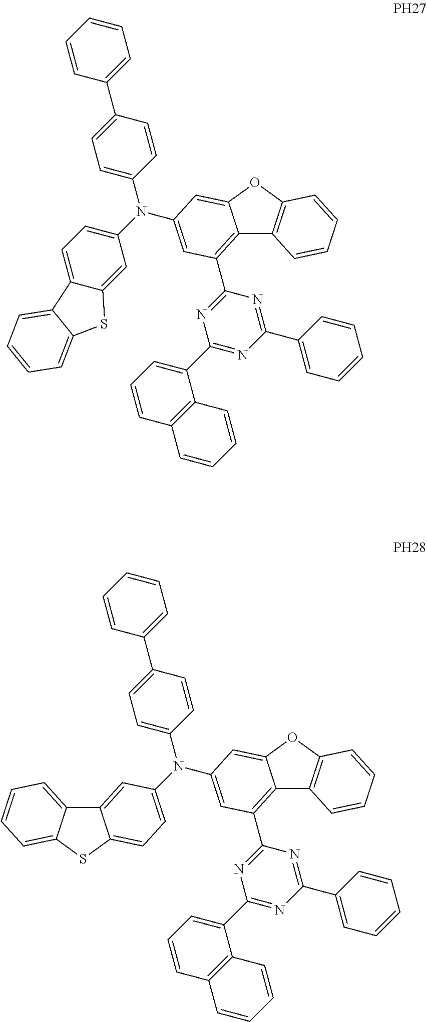 [SMILES: c1ccc(-c2ccc(N(c3cc(-c4nc(-c5ccccc5)nc(-c5cccc6ccccc56)n4)c4c(c3)oc3ccccc34)c3ccc4c(c3)sc3ccccc34)cc2)cc1 ; c1ccc(-c2ccc(N(c3cc(-c4nc(-c5ccccc5)nc(-c5cccc6ccccc56)n4)c4c(c3)oc3ccccc34)c3ccc4sc5ccccc5c4c3)cc2)cc1]

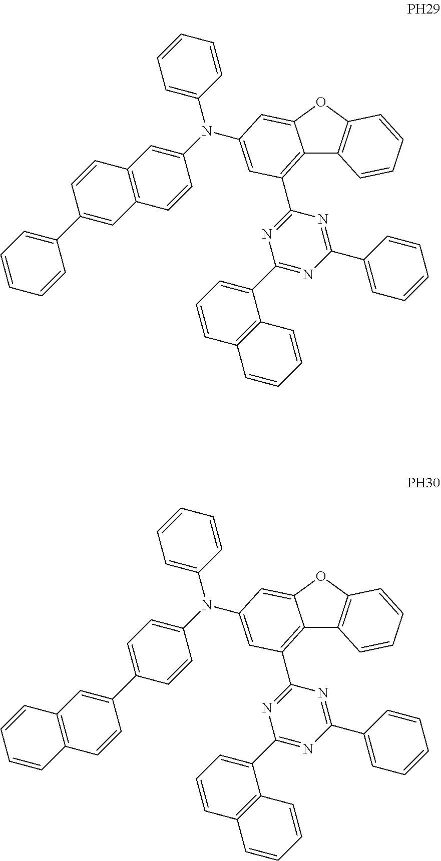 [SMILES: c1ccc(-c2ccc3cc(N(c4ccccc4)c4cc(-c5nc(-c6ccccc6)nc(-c6cccc7ccccc67)n5)c5c(c4)oc4ccccc45)ccc3c2)cc1 ; c1ccc(-c2nc(-c3cccc4ccccc34)nc(-c3cc(N(c4ccccc4)c4ccc(-c5ccc6ccccc6c5)cc4)cc4oc5ccccc5c34)n2)cc1]

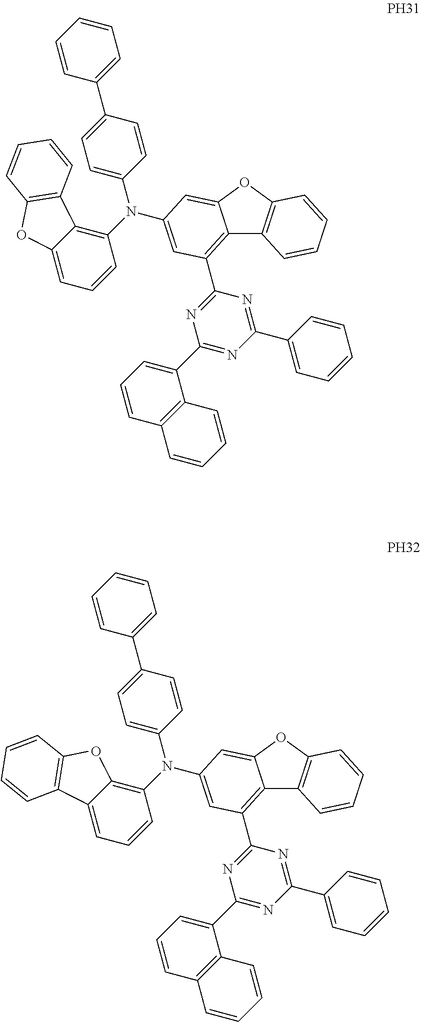 [SMILES: c1ccc(-c2ccc(N(c3cc(-c4nc(-c5ccccc5)nc(-c5cccc6ccccc56)n4)c4c(c3)oc3ccccc34)c3cccc4oc5ccccc5c34)cc2)cc1 ; c1ccc(-c2ccc(N(c3cc(-c4nc(-c5ccccc5)nc(-c5cccc6ccccc56)n4)c4c(c3)oc3ccccc34)c3cccc4c3oc3ccccc34)cc2)cc1]

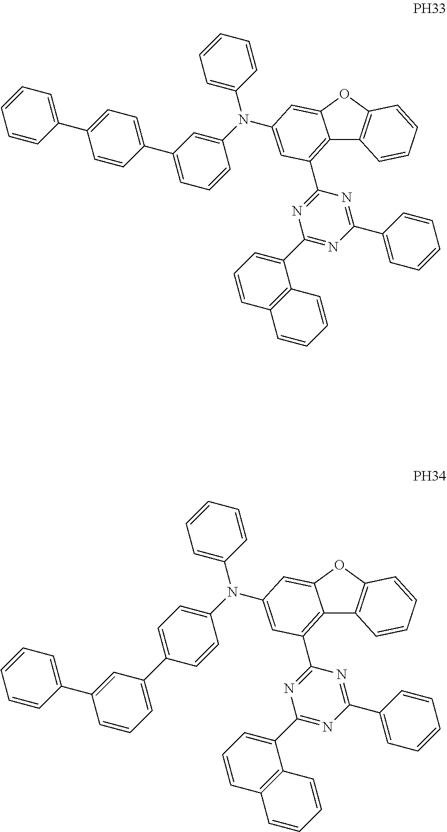 [SMILES: c1ccc(-c2ccc(-c3cccc(N(c4ccccc4)c4cc(-c5nc(-c6ccccc6)nc(-c6cccc7ccccc67)n5)c5c(c4)oc4ccccc45)c3)cc2)cc1 ; c1ccc(-c2cccc(-c3ccc(N(c4ccccc4)c4cc(-c5nc(-c6ccccc6)nc(-c6cccc7ccccc67)n5)c5c(c4)oc4ccccc45)cc3)c2)cc1]

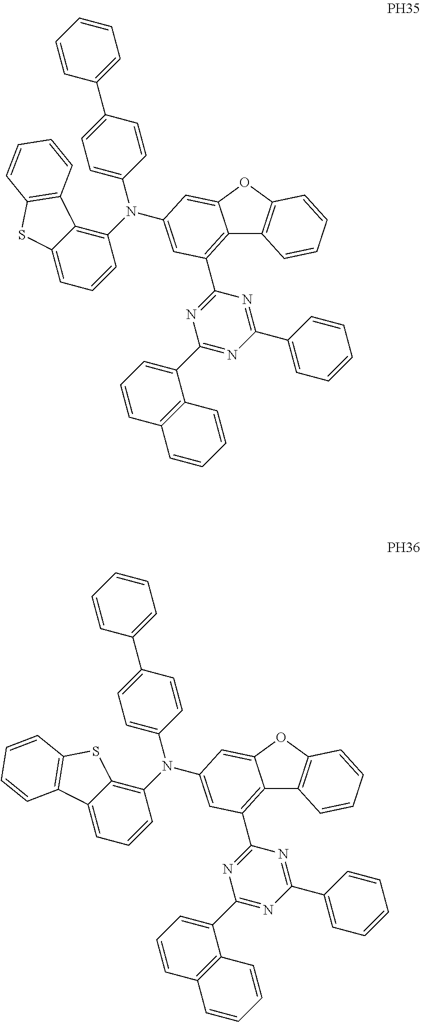 [SMILES: c1ccc(-c2ccc(N(c3cc(-c4nc(-c5ccccc5)nc(-c5cccc6ccccc56)n4)c4c(c3)oc3ccccc34)c3cccc4sc5ccccc5c34)cc2)cc1 ; c1ccc(-c2ccc(N(c3cc(-c4nc(-c5ccccc5)nc(-c5cccc6ccccc56)n4)c4c(c3)oc3ccccc34)c3cccc4c3sc3ccccc34)cc2)cc1]

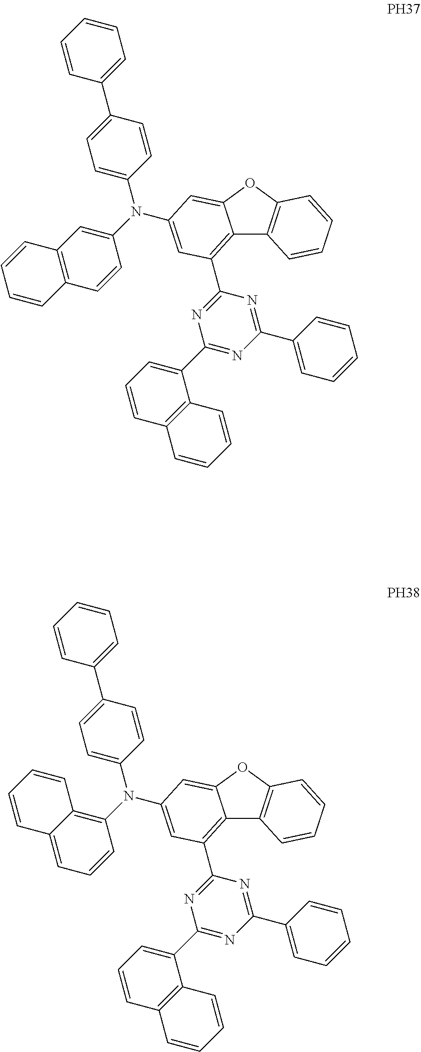 [SMILES: c1ccc(-c2ccc(N(c3ccc4ccccc4c3)c3cc(-c4nc(-c5ccccc5)nc(-c5cccc6ccccc56)n4)c4c(c3)oc3ccccc34)cc2)cc1 ; c1ccc(-c2ccc(N(c3cc(-c4nc(-c5ccccc5)nc(-c5cccc6ccccc56)n4)c4c(c3)oc3ccccc34)c3cccc4ccccc34)cc2)cc1]

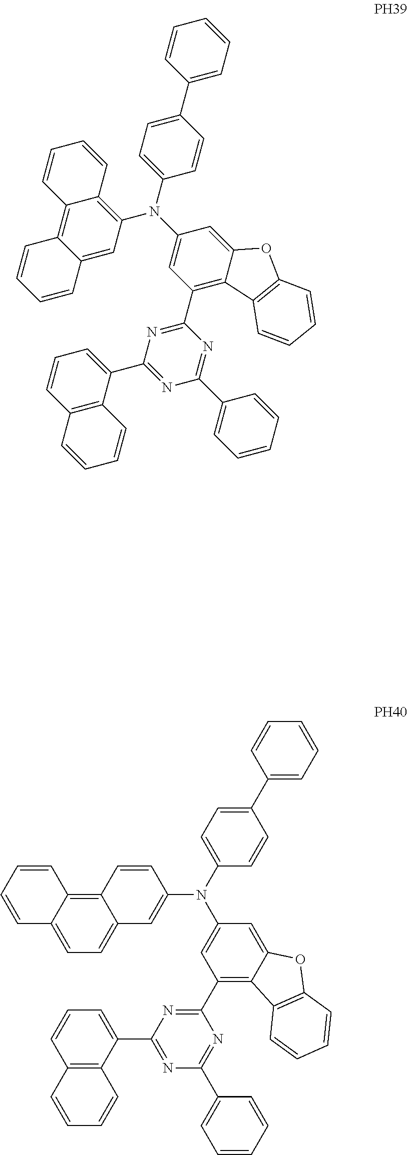 [SMILES: c1ccc(-c2ccc(N(c3cc(-c4nc(-c5ccccc5)nc(-c5cccc6ccccc56)n4)c4c(c3)oc3ccccc34)c3cc4ccccc4c4ccccc34)cc2)cc1 ; c1ccc(-c2ccc(N(c3ccc4c(ccc5ccccc54)c3)c3cc(-c4nc(-c5ccccc5)nc(-c5cccc6ccccc56)n4)c4c(c3)oc3ccccc34)cc2)cc1]

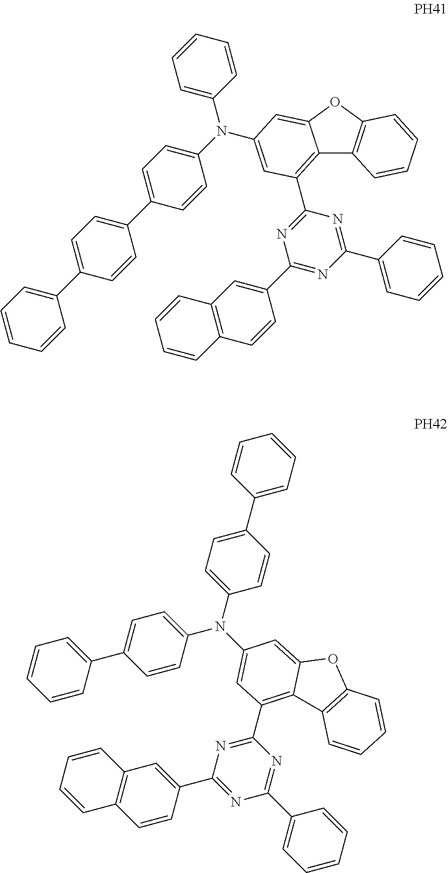 [SMILES: c1ccc(-c2ccc(-c3ccc(N(c4ccccc4)c4cc(-c5nc(-c6ccccc6)nc(-c6ccc7ccccc7c6)n5)c5c(c4)oc4ccccc45)cc3)cc2)cc1 ; c1ccc(-c2ccc(N(c3ccc(-c4ccccc4)cc3)c3cc(-c4nc(-c5ccccc5)nc(-c5ccc6ccccc6c5)n4)c4c(c3)oc3ccccc34)cc2)cc1]

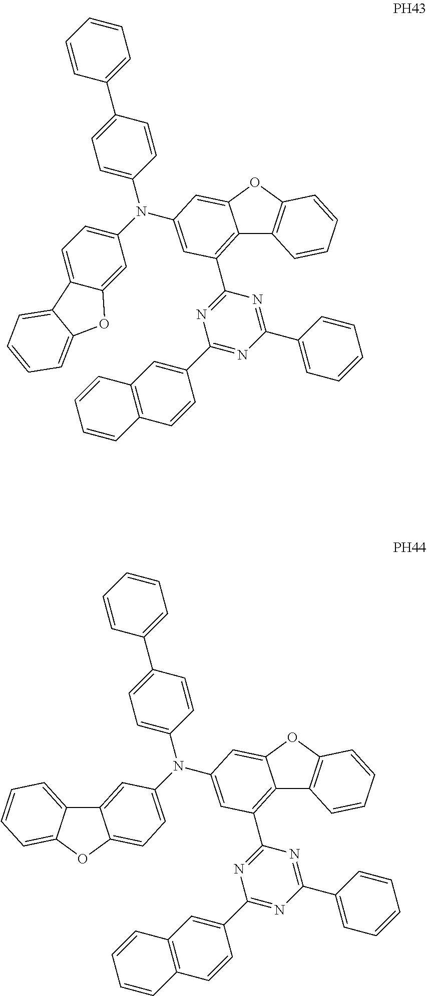 [SMILES: c1ccc(-c2ccc(N(c3ccc4c(c3)oc3ccccc34)c3cc(-c4nc(-c5ccccc5)nc(-c5ccc6ccccc6c5)n4)c4c(c3)oc3ccccc34)cc2)cc1 ; c1ccc(-c2ccc(N(c3cc(-c4nc(-c5ccccc5)nc(-c5ccc6ccccc6c5)n4)c4c(c3)oc3ccccc34)c3ccc4oc5ccccc5c4c3)cc2)cc1]

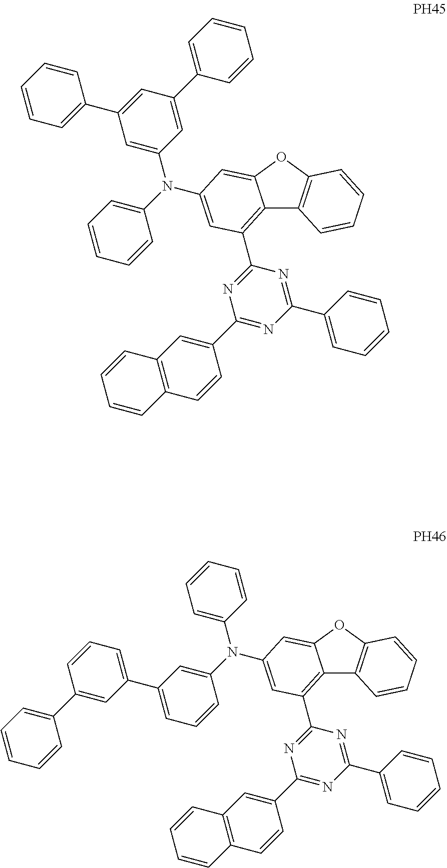 [SMILES: c1ccc(-c2cc(-c3ccccc3)cc(N(c3ccccc3)c3cc(-c4nc(-c5ccccc5)nc(-c5ccc6ccccc6c5)n4)c4c(c3)oc3ccccc34)c2)cc1 ; c1ccc(-c2cccc(-c3cccc(N(c4ccccc4)c4cc(-c5nc(-c6ccccc6)nc(-c6ccc7ccccc7c6)n5)c5c(c4)oc4ccccc45)c3)c2)cc1]

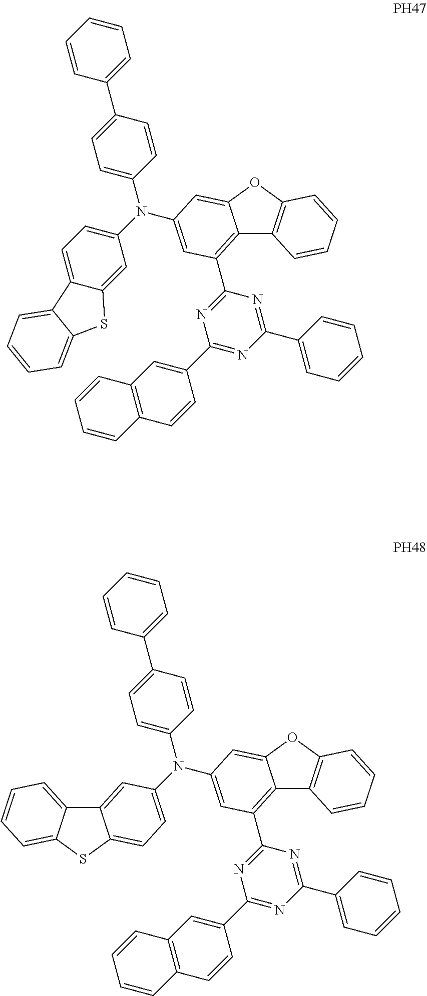 [SMILES: c1ccc(-c2ccc(N(c3cc(-c4nc(-c5ccccc5)nc(-c5ccc6ccccc6c5)n4)c4c(c3)oc3ccccc34)c3ccc4c(c3)sc3ccccc34)cc2)cc1 ; c1ccc(-c2ccc(N(c3cc(-c4nc(-c5ccccc5)nc(-c5ccc6ccccc6c5)n4)c4c(c3)oc3ccccc34)c3ccc4sc5ccccc5c4c3)cc2)cc1]

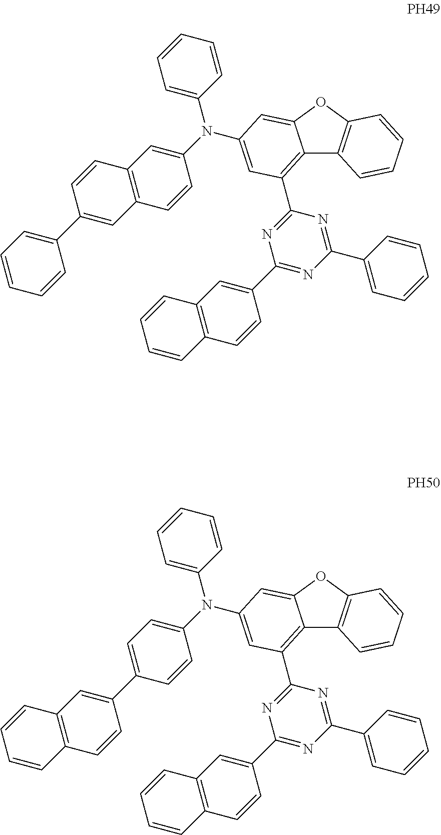 [SMILES: c1ccc(-c2ccc3cc(N(c4ccccc4)c4cc(-c5nc(-c6ccccc6)nc(-c6ccc7ccccc7c6)n5)c5c(c4)oc4ccccc45)ccc3c2)cc1 ; c1ccc(-c2nc(-c3ccc4ccccc4c3)nc(-c3cc(N(c4ccccc4)c4ccc(-c5ccc6ccccc6c5)cc4)cc4oc5ccccc5c34)n2)cc1]

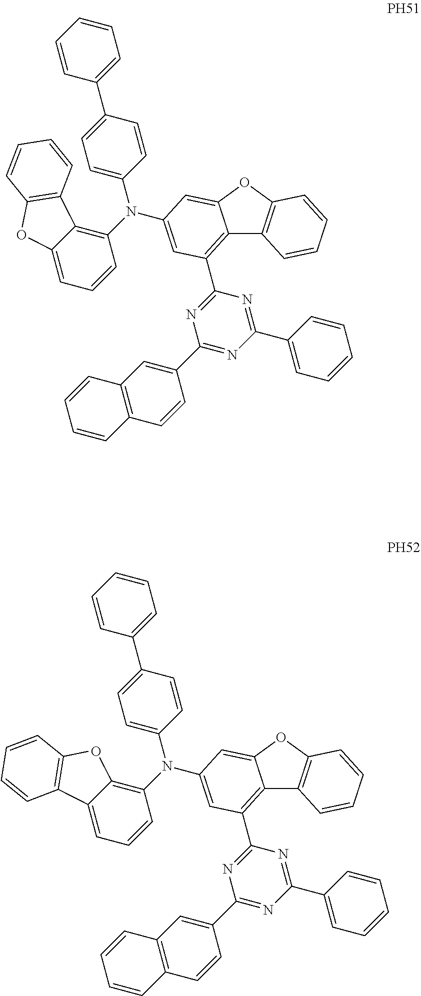 [SMILES: c1ccc(-c2ccc(N(c3cc(-c4nc(-c5ccccc5)nc(-c5ccc6ccccc6c5)n4)c4c(c3)oc3ccccc34)c3cccc4oc5ccccc5c34)cc2)cc1 ; c1ccc(-c2ccc(N(c3cc(-c4nc(-c5ccccc5)nc(-c5ccc6ccccc6c5)n4)c4c(c3)oc3ccccc34)c3cccc4c3oc3ccccc34)cc2)cc1]

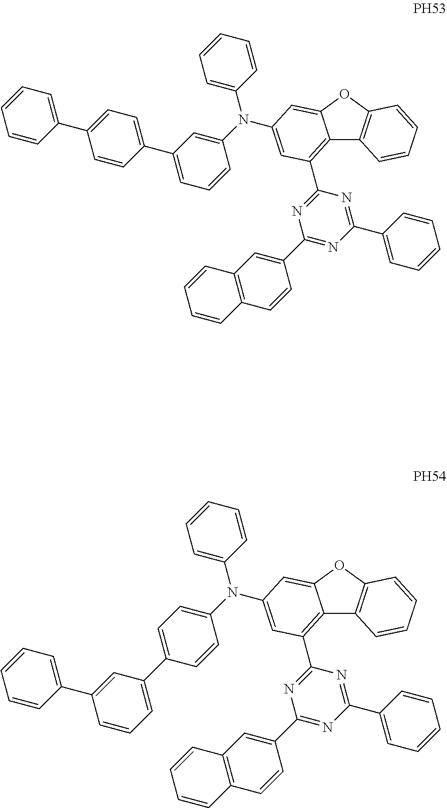 [SMILES: c1ccc(-c2ccc(-c3cccc(N(c4ccccc4)c4cc(-c5nc(-c6ccccc6)nc(-c6ccc7ccccc7c6)n5)c5c(c4)oc4ccccc45)c3)cc2)cc1 ; c1ccc(-c2cccc(-c3ccc(N(c4ccccc4)c4cc(-c5nc(-c6ccccc6)nc(-c6ccc7ccccc7c6)n5)c5c(c4)oc4ccccc45)cc3)c2)cc1]

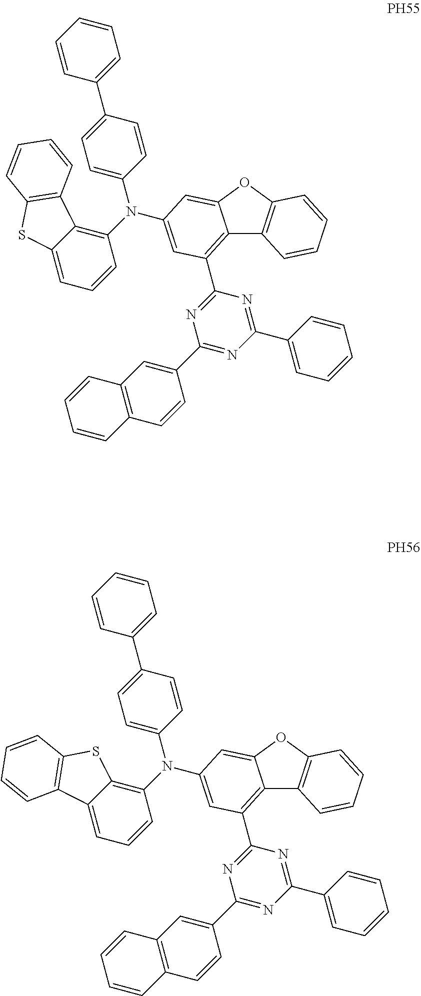 [SMILES: c1ccc(-c2ccc(N(c3cc(-c4nc(-c5ccccc5)nc(-c5ccc6ccccc6c5)n4)c4c(c3)oc3ccccc34)c3cccc4sc5ccccc5c34)cc2)cc1 ; c1ccc(-c2ccc(N(c3cc(-c4nc(-c5ccccc5)nc(-c5ccc6ccccc6c5)n4)c4c(c3)oc3ccccc34)c3cccc4c3sc3ccccc34)cc2)cc1]

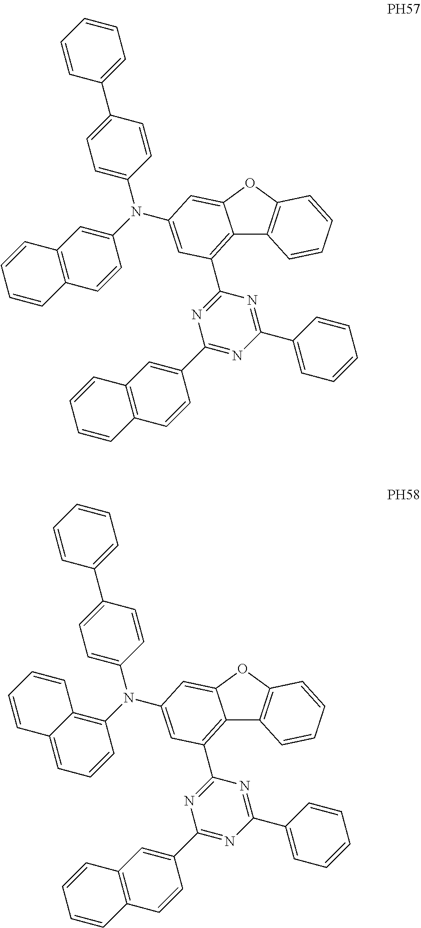 [SMILES: c1ccc(-c2ccc(N(c3ccc4ccccc4c3)c3cc(-c4nc(-c5ccccc5)nc(-c5ccc6ccccc6c5)n4)c4c(c3)oc3ccccc34)cc2)cc1 ; c1ccc(-c2ccc(N(c3cc(-c4nc(-c5ccccc5)nc(-c5ccc6ccccc6c5)n4)c4c(c3)oc3ccccc34)c3cccc4ccccc34)cc2)cc1]

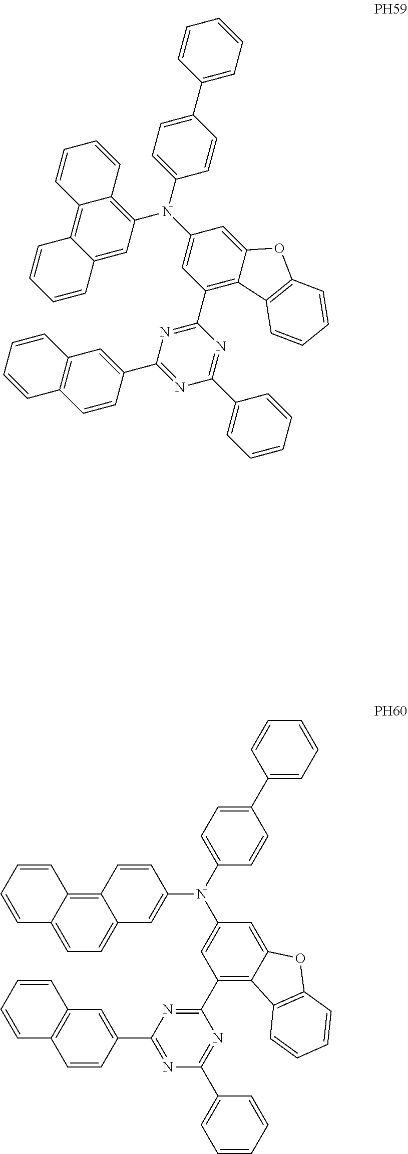 [SMILES: c1ccc(-c2ccc(N(c3cc(-c4nc(-c5ccccc5)nc(-c5ccc6ccccc6c5)n4)c4c(c3)oc3ccccc34)c3cc4ccccc4c4ccccc34)cc2)cc1 ; c1ccc(-c2ccc(N(c3ccc4c(ccc5ccccc54)c3)c3cc(-c4nc(-c5ccccc5)nc(-c5ccc6ccccc6c5)n4)c4c(c3)oc3ccccc34)cc2)cc1]

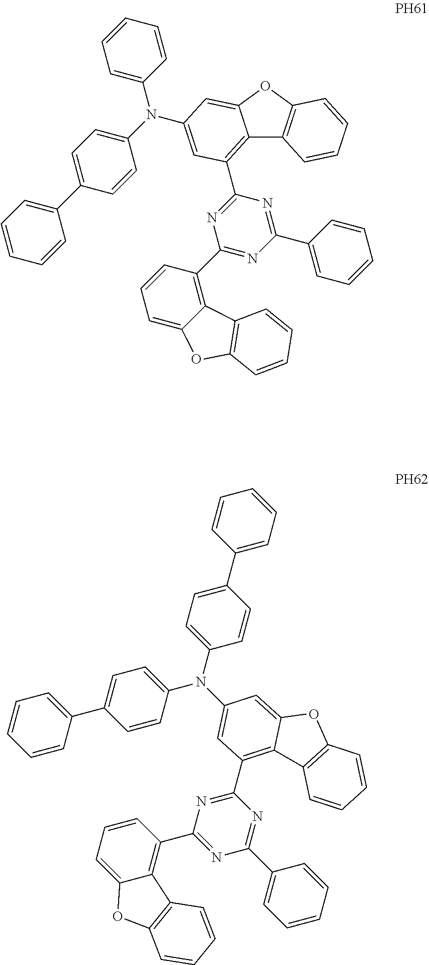 [SMILES: c1ccc(-c2ccc(N(c3ccccc3)c3cc(-c4nc(-c5ccccc5)nc(-c5cccc6oc7ccccc7c56)n4)c4c(c3)oc3ccccc34)cc2)cc1 ; c1ccc(-c2ccc(N(c3ccc(-c4ccccc4)cc3)c3cc(-c4nc(-c5ccccc5)nc(-c5cccc6oc7ccccc7c56)n4)c4c(c3)oc3ccccc34)cc2)cc1]

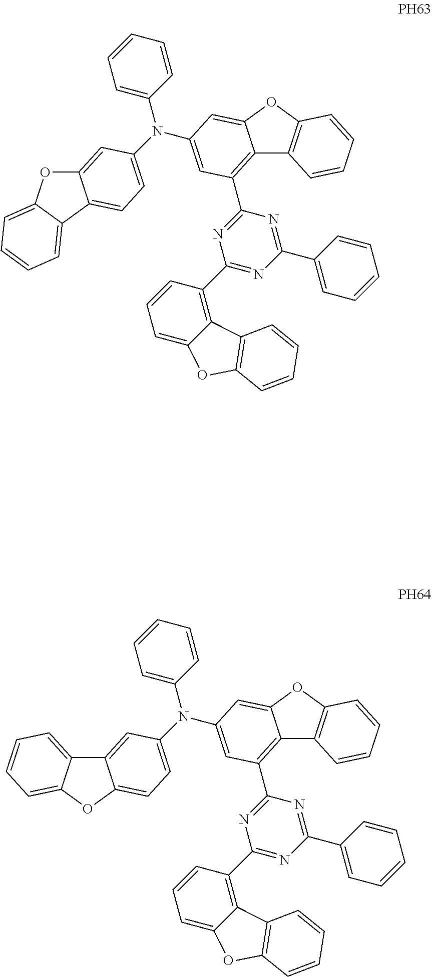 [SMILES: c1ccc(-c2nc(-c3cccc4oc5ccccc5c34)nc(-c3cc(N(c4ccccc4)c4ccc5c(c4)oc4ccccc45)cc4oc5ccccc5c34)n2)cc1 ; c1ccc(-c2nc(-c3cccc4oc5ccccc5c34)nc(-c3cc(N(c4ccccc4)c4ccc5oc6ccccc6c5c4)cc4oc5ccccc5c34)n2)cc1]

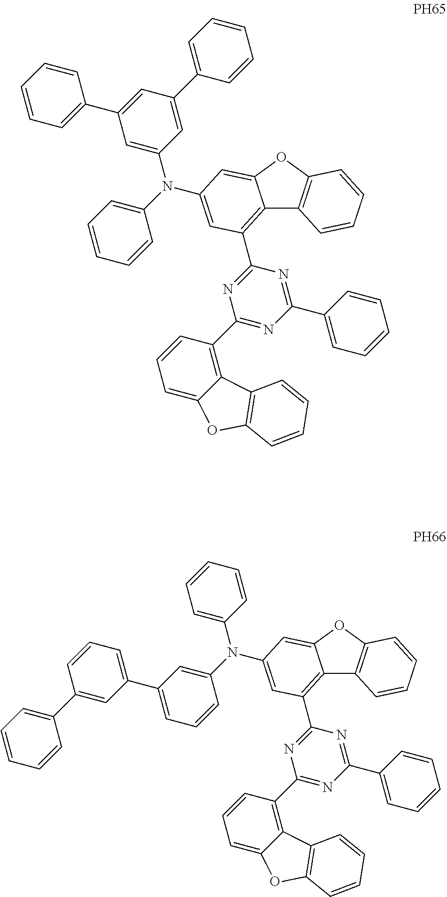 [SMILES: c1ccc(-c2cc(-c3ccccc3)cc(N(c3ccccc3)c3cc(-c4nc(-c5ccccc5)nc(-c5cccc6oc7ccccc7c56)n4)c4c(c3)oc3ccccc34)c2)cc1 ; c1ccc(-c2cccc(-c3cccc(N(c4ccccc4)c4cc(-c5nc(-c6ccccc6)nc(-c6cccc7oc8ccccc8c67)n5)c5c(c4)oc4ccccc45)c3)c2)cc1]

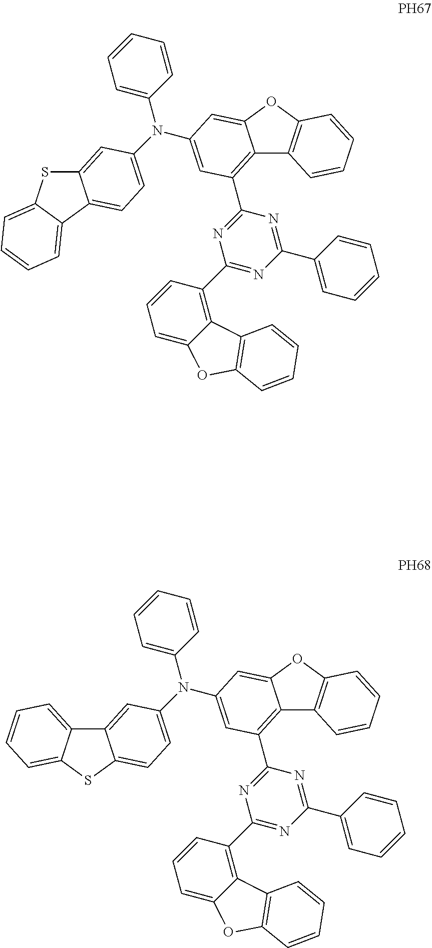 [SMILES: c1ccc(-c2nc(-c3cccc4oc5ccccc5c34)nc(-c3cc(N(c4ccccc4)c4ccc5c(c4)sc4ccccc45)cc4oc5ccccc5c34)n2)cc1 ; c1ccc(-c2nc(-c3cccc4oc5ccccc5c34)nc(-c3cc(N(c4ccccc4)c4ccc5sc6ccccc6c5c4)cc4oc5ccccc5c34)n2)cc1]

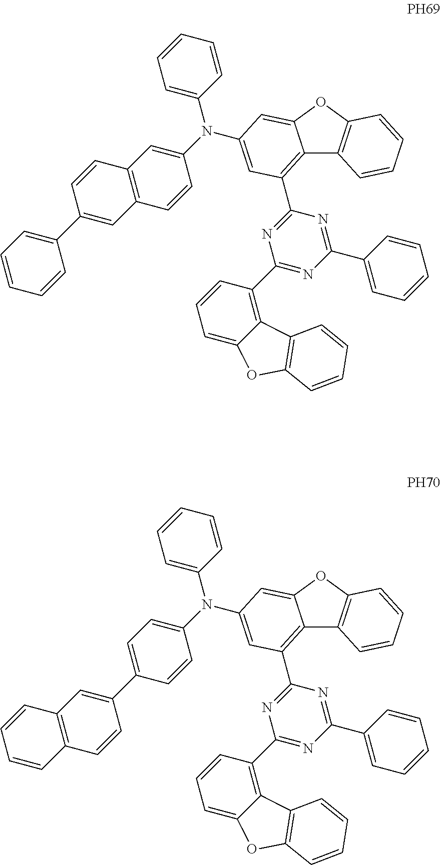 [SMILES: c1ccc(-c2ccc3cc(N(c4ccccc4)c4cc(-c5nc(-c6ccccc6)nc(-c6cccc7oc8ccccc8c67)n5)c5c(c4)oc4ccccc45)ccc3c2)cc1 ; c1ccc(-c2nc(-c3cccc4oc5ccccc5c34)nc(-c3cc(N(c4ccccc4)c4ccc(-c5ccc6ccccc6c5)cc4)cc4oc5ccccc5c34)n2)cc1]

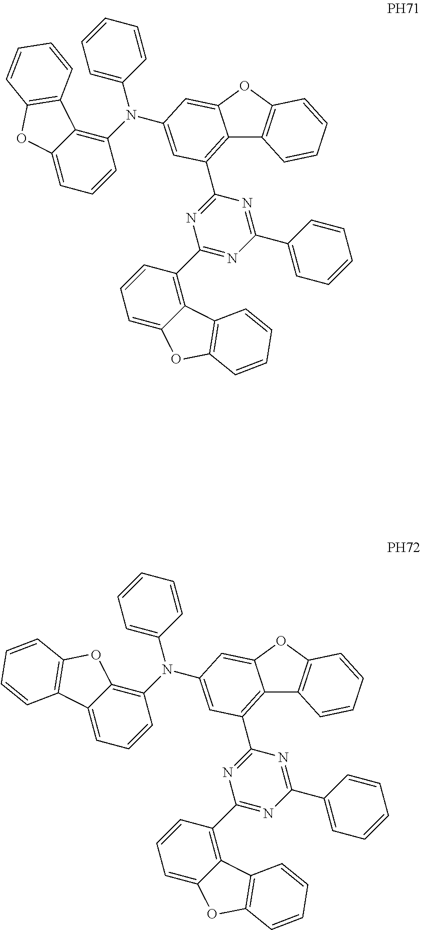 [SMILES: c1ccc(-c2nc(-c3cccc4oc5ccccc5c34)nc(-c3cc(N(c4ccccc4)c4cccc5oc6ccccc6c45)cc4oc5ccccc5c34)n2)cc1 ; c1ccc(-c2nc(-c3cccc4oc5ccccc5c34)nc(-c3cc(N(c4ccccc4)c4cccc5c4oc4ccccc45)cc4oc5ccccc5c34)n2)cc1]

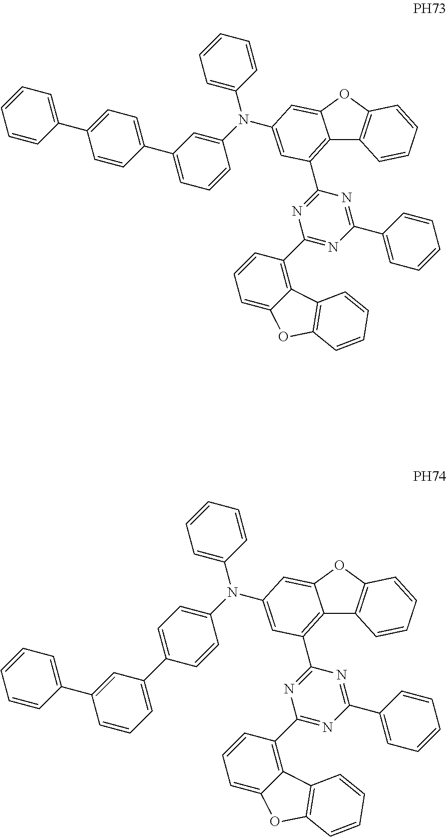 [SMILES: c1ccc(-c2ccc(-c3cccc(N(c4ccccc4)c4cc(-c5nc(-c6ccccc6)nc(-c6cccc7oc8ccccc8c67)n5)c5c(c4)oc4ccccc45)c3)cc2)cc1 ; c1ccc(-c2cccc(-c3ccc(N(c4ccccc4)c4cc(-c5nc(-c6ccccc6)nc(-c6cccc7oc8ccccc8c67)n5)c5c(c4)oc4ccccc45)cc3)c2)cc1]

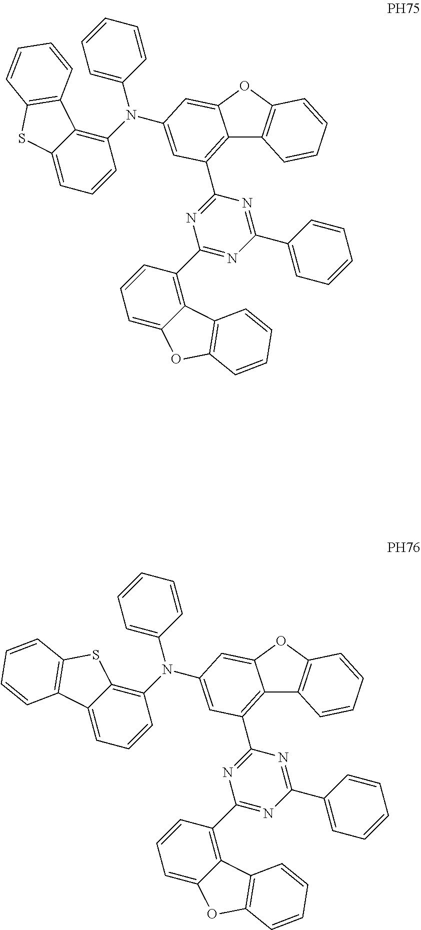 [SMILES: c1ccc(-c2nc(-c3cccc4oc5ccccc5c34)nc(-c3cc(N(c4ccccc4)c4cccc5sc6ccccc6c45)cc4oc5ccccc5c34)n2)cc1 ; c1ccc(-c2nc(-c3cccc4oc5ccccc5c34)nc(-c3cc(N(c4ccccc4)c4cccc5c4sc4ccccc45)cc4oc5ccccc5c34)n2)cc1]

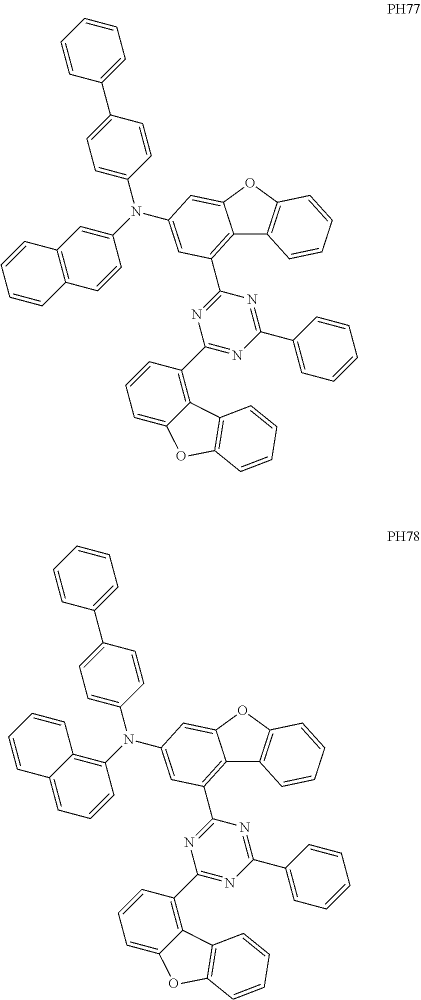 [SMILES: c1ccc(-c2ccc(N(c3ccc4ccccc4c3)c3cc(-c4nc(-c5ccccc5)nc(-c5cccc6oc7ccccc7c56)n4)c4c(c3)oc3ccccc34)cc2)cc1 ; c1ccc(-c2ccc(N(c3cc(-c4nc(-c5ccccc5)nc(-c5cccc6oc7ccccc7c56)n4)c4c(c3)oc3ccccc34)c3cccc4ccccc34)cc2)cc1]

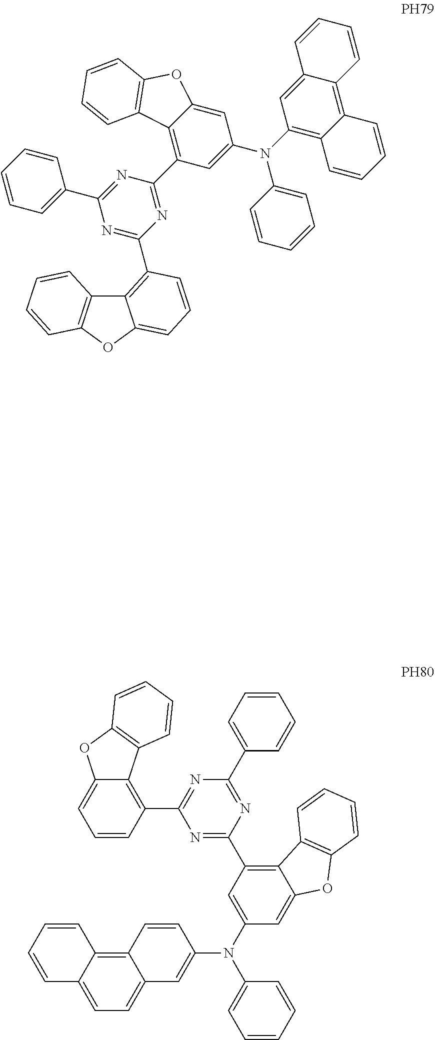 [SMILES: c1ccc(-c2nc(-c3cccc4oc5ccccc5c34)nc(-c3cc(N(c4ccccc4)c4cc5ccccc5c5ccccc45)cc4oc5ccccc5c34)n2)cc1 ; c1ccc(-c2nc(-c3cccc4oc5ccccc5c34)nc(-c3cc(N(c4ccccc4)c4ccc5c(ccc6ccccc65)c4)cc4oc5ccccc5c34)n2)cc1]

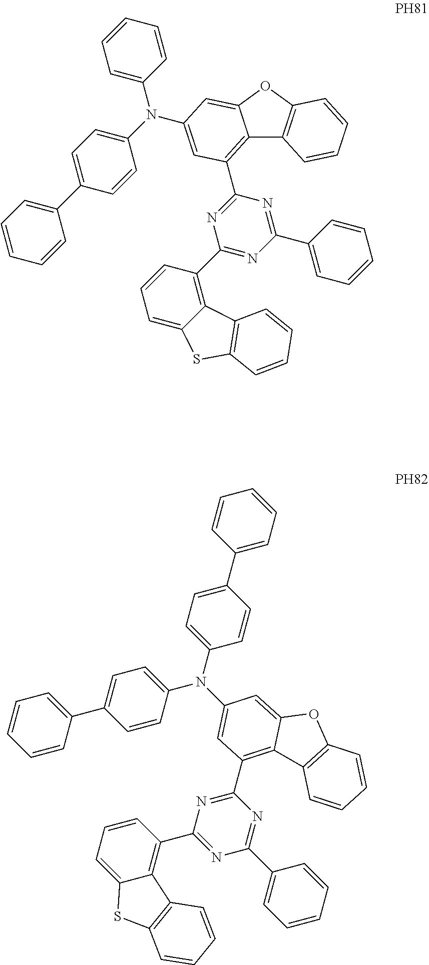 [SMILES: c1ccc(-c2ccc(N(c3ccccc3)c3cc(-c4nc(-c5ccccc5)nc(-c5cccc6sc7ccccc7c56)n4)c4c(c3)oc3ccccc34)cc2)cc1 ; c1ccc(-c2ccc(N(c3ccc(-c4ccccc4)cc3)c3cc(-c4nc(-c5ccccc5)nc(-c5cccc6sc7ccccc7c56)n4)c4c(c3)oc3ccccc34)cc2)cc1]

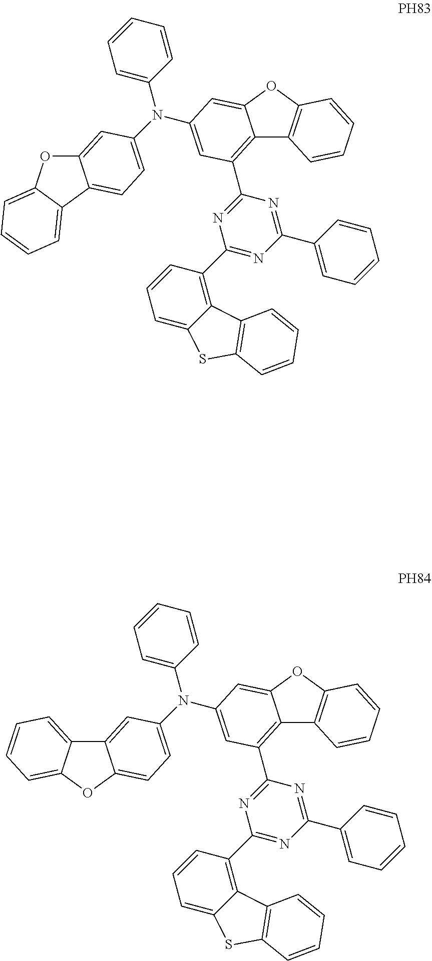 [SMILES: c1ccc(-c2nc(-c3cc(N(c4ccccc4)c4ccc5c(c4)oc4ccccc45)cc4oc5ccccc5c34)nc(-c3cccc4sc5ccccc5c34)n2)cc1 ; c1ccc(-c2nc(-c3cc(N(c4ccccc4)c4ccc5oc6ccccc6c5c4)cc4oc5ccccc5c34)nc(-c3cccc4sc5ccccc5c34)n2)cc1]

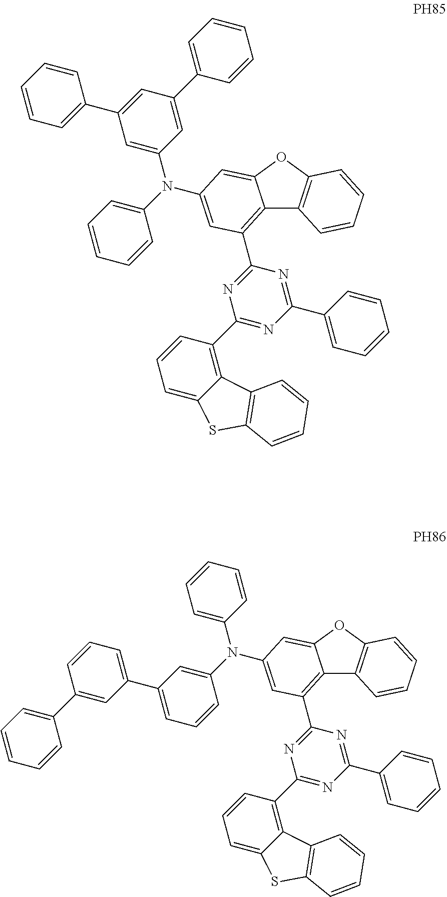 [SMILES: c1ccc(-c2cc(-c3ccccc3)cc(N(c3ccccc3)c3cc(-c4nc(-c5ccccc5)nc(-c5cccc6sc7ccccc7c56)n4)c4c(c3)oc3ccccc34)c2)cc1 ; c1ccc(-c2cccc(-c3cccc(N(c4ccccc4)c4cc(-c5nc(-c6ccccc6)nc(-c6cccc7sc8ccccc8c67)n5)c5c(c4)oc4ccccc45)c3)c2)cc1]

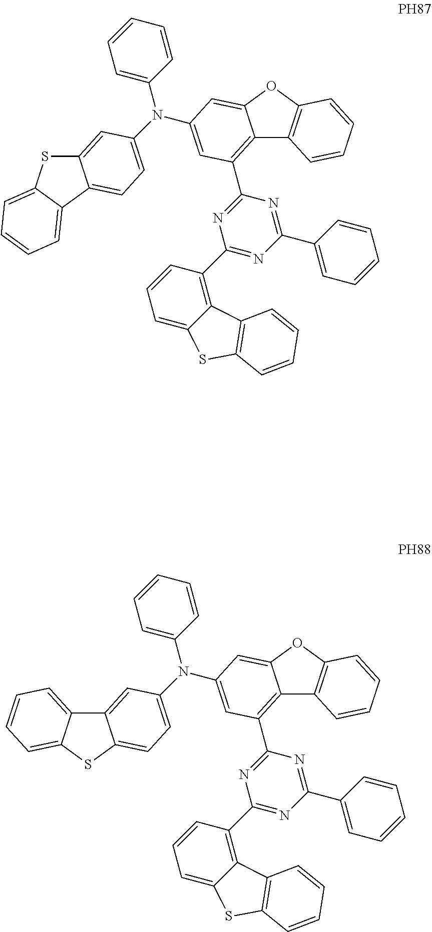 [SMILES: c1ccc(-c2nc(-c3cc(N(c4ccccc4)c4ccc5c(c4)sc4ccccc45)cc4oc5ccccc5c34)nc(-c3cccc4sc5ccccc5c34)n2)cc1 ; c1ccc(-c2nc(-c3cc(N(c4ccccc4)c4ccc5sc6ccccc6c5c4)cc4oc5ccccc5c34)nc(-c3cccc4sc5ccccc5c34)n2)cc1]

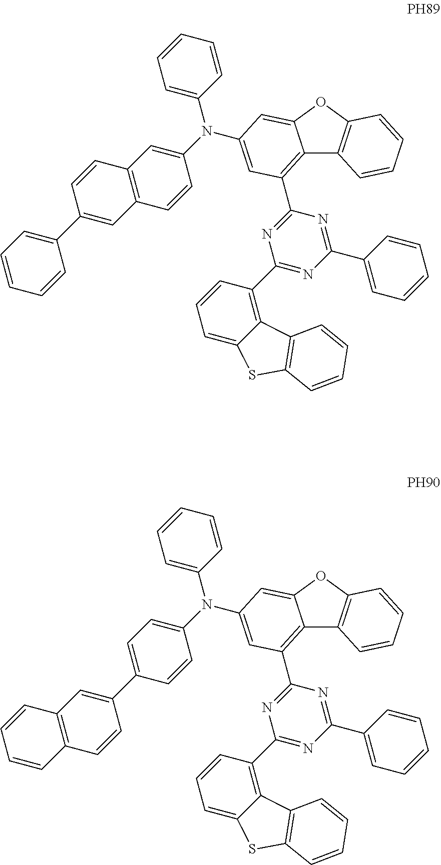 [SMILES: c1ccc(-c2ccc3cc(N(c4ccccc4)c4cc(-c5nc(-c6ccccc6)nc(-c6cccc7sc8ccccc8c67)n5)c5c(c4)oc4ccccc45)ccc3c2)cc1 ; c1ccc(-c2nc(-c3cc(N(c4ccccc4)c4ccc(-c5ccc6ccccc6c5)cc4)cc4oc5ccccc5c34)nc(-c3cccc4sc5ccccc5c34)n2)cc1]

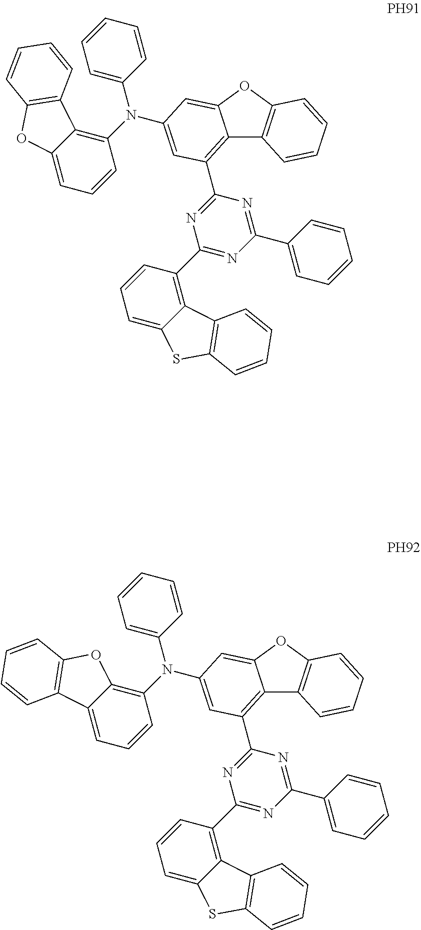 [SMILES: c1ccc(-c2nc(-c3cc(N(c4ccccc4)c4cccc5oc6ccccc6c45)cc4oc5ccccc5c34)nc(-c3cccc4sc5ccccc5c34)n2)cc1 ; c1ccc(-c2nc(-c3cc(N(c4ccccc4)c4cccc5c4oc4ccccc45)cc4oc5ccccc5c34)nc(-c3cccc4sc5ccccc5c34)n2)cc1]

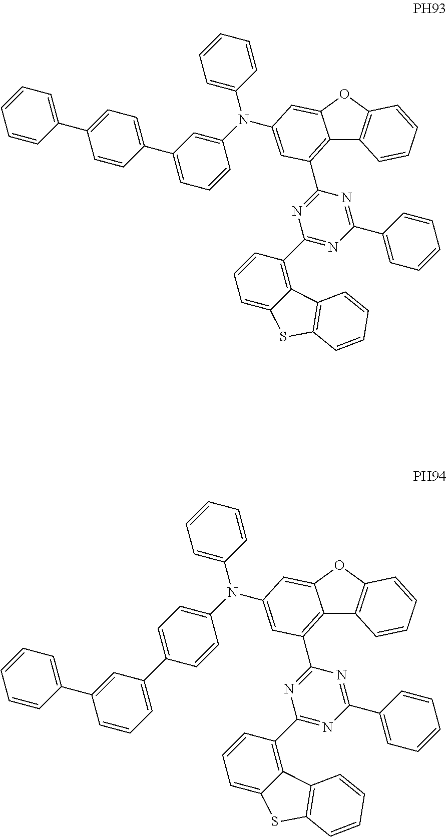 [SMILES: c1ccc(-c2ccc(-c3cccc(N(c4ccccc4)c4cc(-c5nc(-c6ccccc6)nc(-c6cccc7sc8ccccc8c67)n5)c5c(c4)oc4ccccc45)c3)cc2)cc1 ; c1ccc(-c2cccc(-c3ccc(N(c4ccccc4)c4cc(-c5nc(-c6ccccc6)nc(-c6cccc7sc8ccccc8c67)n5)c5c(c4)oc4ccccc45)cc3)c2)cc1]

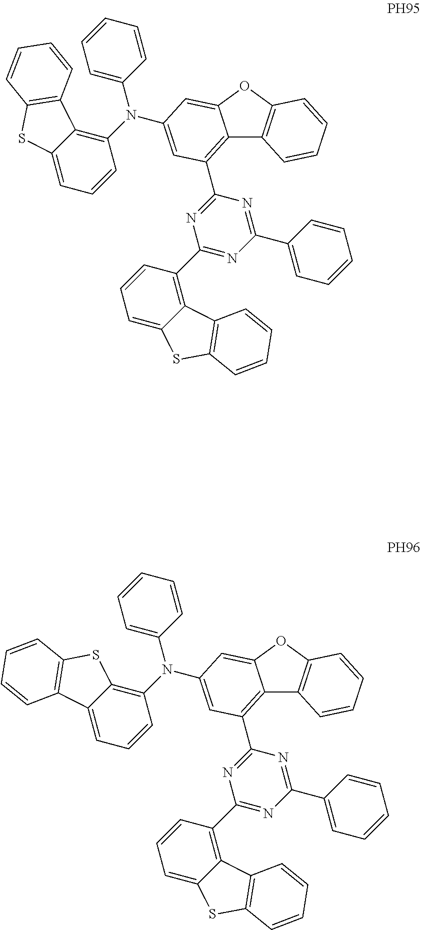 [SMILES: c1ccc(-c2nc(-c3cc(N(c4ccccc4)c4cccc5sc6ccccc6c45)cc4oc5ccccc5c34)nc(-c3cccc4sc5ccccc5c34)n2)cc1 ; c1ccc(-c2nc(-c3cc(N(c4ccccc4)c4cccc5c4sc4ccccc45)cc4oc5ccccc5c34)nc(-c3cccc4sc5ccccc5c34)n2)cc1]

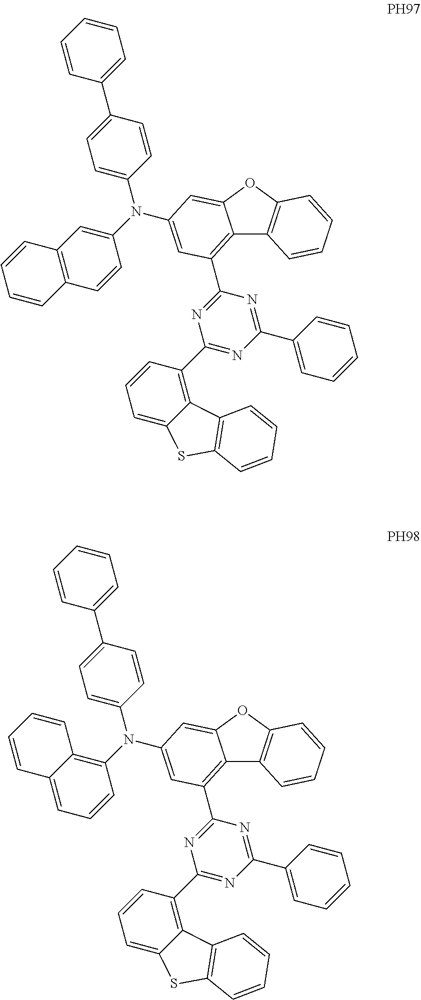 [SMILES: c1ccc(-c2ccc(N(c3ccc4ccccc4c3)c3cc(-c4nc(-c5ccccc5)nc(-c5cccc6sc7ccccc7c56)n4)c4c(c3)oc3ccccc34)cc2)cc1 ; c1ccc(-c2ccc(N(c3cc(-c4nc(-c5ccccc5)nc(-c5cccc6sc7ccccc7c56)n4)c4c(c3)oc3ccccc34)c3cccc4ccccc34)cc2)cc1]

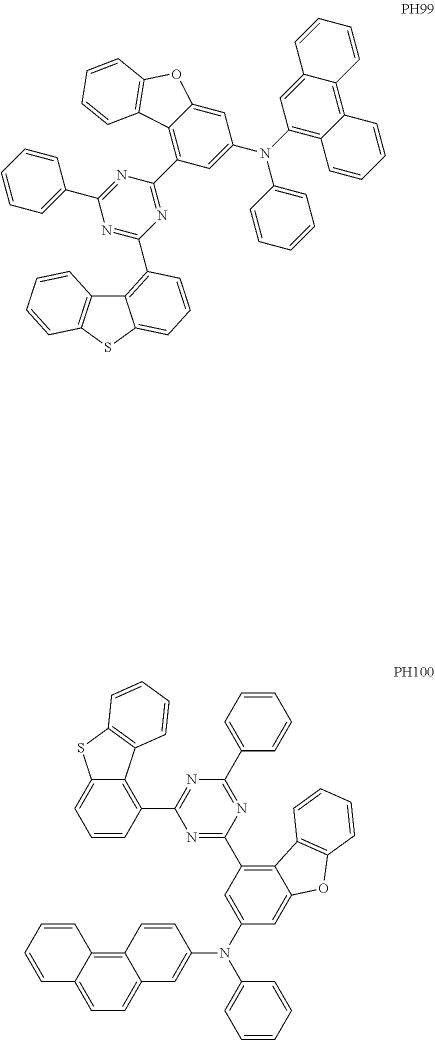 [SMILES: c1ccc(-c2nc(-c3cc(N(c4ccccc4)c4cc5ccccc5c5ccccc45)cc4oc5ccccc5c34)nc(-c3cccc4sc5ccccc5c34)n2)cc1 ; c1ccc(-c2nc(-c3cc(N(c4ccccc4)c4ccc5c(ccc6ccccc65)c4)cc4oc5ccccc5c34)nc(-c3cccc4sc5ccccc5c34)n2)cc1]

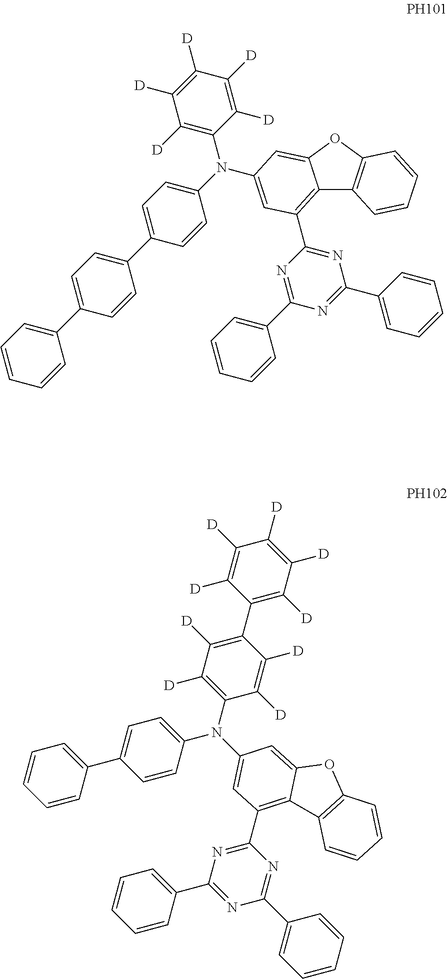 [SMILES: [2H]c1c([2H])c([2H])c(N(c2ccc(-c3ccc(-c4ccccc4)cc3)cc2)c2cc(-c3nc(-c4ccccc4)nc(-c4ccccc4)n3)c3c(c2)oc2ccccc23)c([2H])c1[2H] ; [2H]c1c([2H])c([2H])c(-c2c([2H])c([2H])c(N(c3ccc(-c4ccccc4)cc3)c3cc(-c4nc(-c5ccccc5)nc(-c5ccccc5)n4)c4c(c3)oc3ccccc34)c([2H])c2[2H])c([2H])c1[2H]]

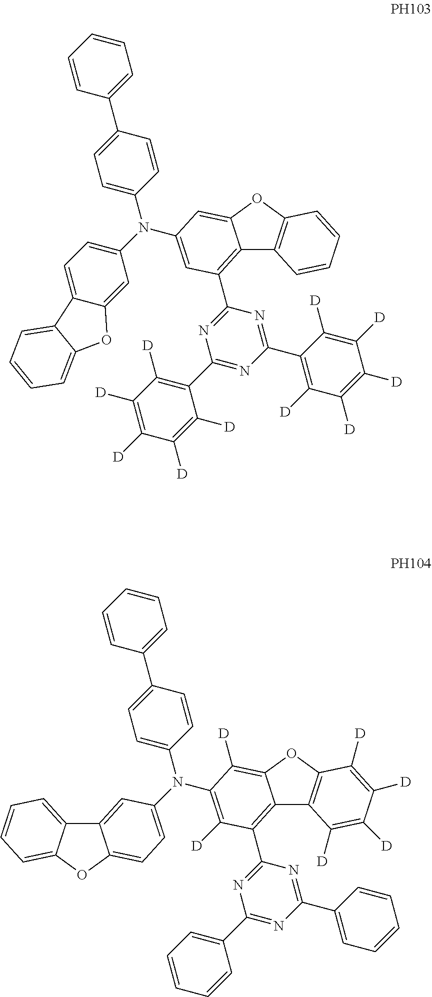 [SMILES: [2H]c1c([2H])c([2H])c(-c2nc(-c3c([2H])c([2H])c([2H])c([2H])c3[2H])nc(-c3cc(N(c4ccc(-c5ccccc5)cc4)c4ccc5c(c4)oc4ccccc45)cc4oc5ccccc5c34)n2)c([2H])c1[2H] ; [2H]c1c([2H])c([2H])c2c(oc3c([2H])c(N(c4ccc(-c5ccccc5)cc4)c4ccc5oc6ccccc6c5c4)c([2H])c(-c4nc(-c5ccccc5)nc(-c5ccccc5)n4)c32)c1[2H]]

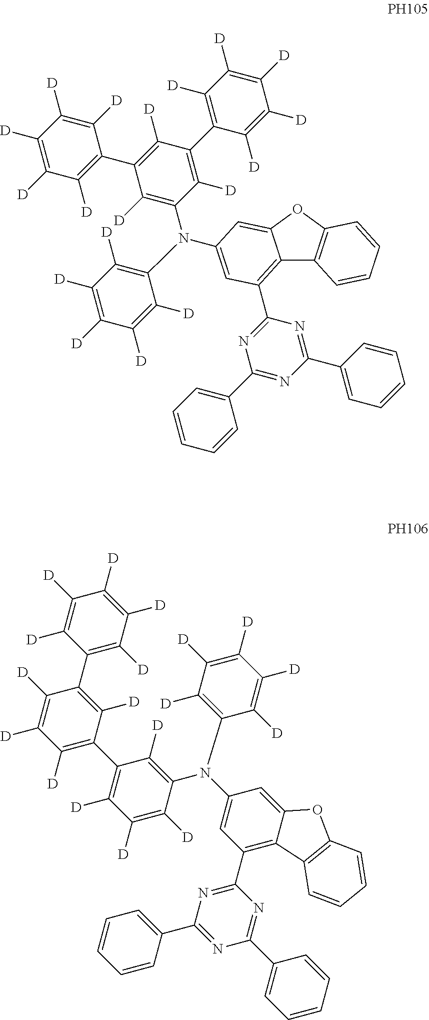 [SMILES: [2H]c1c([2H])c([2H])c(-c2c([2H])c(-c3c([2H])c([2H])c([2H])c([2H])c3[2H])c([2H])c(N(c3cc(-c4nc(-c5ccccc5)nc(-c5ccccc5)n4)c4c(c3)oc3ccccc34)c3c([2H])c([2H])c([2H])c([2H])c3[2H])c2[2H])c([2H])c1[2H] ; [2H]c1c([2H])c([2H])c(-c2c([2H])c([2H])c([2H])c(-c3c([2H])c([2H])c([2H])c(N(c4cc(-c5nc(-c6ccccc6)nc(-c6ccccc6)n5)c5c(c4)oc4ccccc45)c4c([2H])c([2H])c([2H])c([2H])c4[2H])c3[2H])c2[2H])c([2H])c1[2H]]

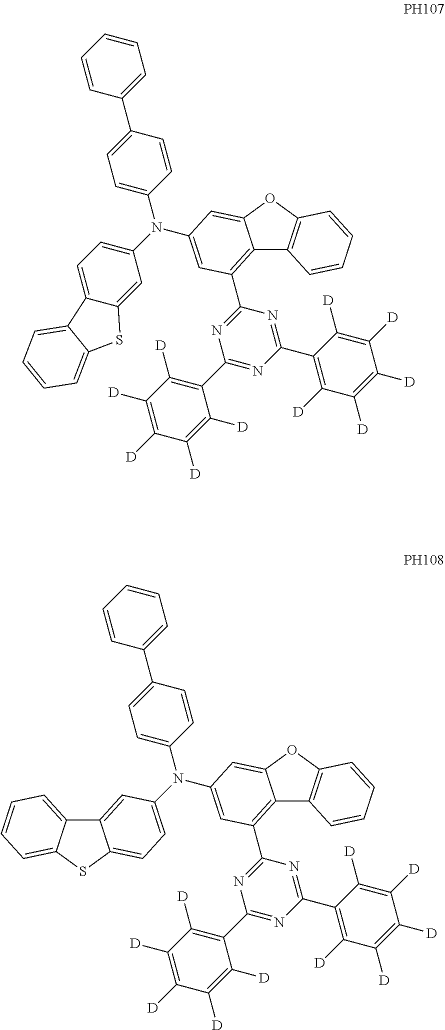 [SMILES: [2H]c1c([2H])c([2H])c(-c2nc(-c3c([2H])c([2H])c([2H])c([2H])c3[2H])nc(-c3cc(N(c4ccc(-c5ccccc5)cc4)c4ccc5c(c4)sc4ccccc45)cc4oc5ccccc5c34)n2)c([2H])c1[2H] ; [2H]c1c([2H])c([2H])c(-c2nc(-c3c([2H])c([2H])c([2H])c([2H])c3[2H])nc(-c3cc(N(c4ccc(-c5ccccc5)cc4)c4ccc5sc6ccccc6c5c4)cc4oc5ccccc5c34)n2)c([2H])c1[2H]]

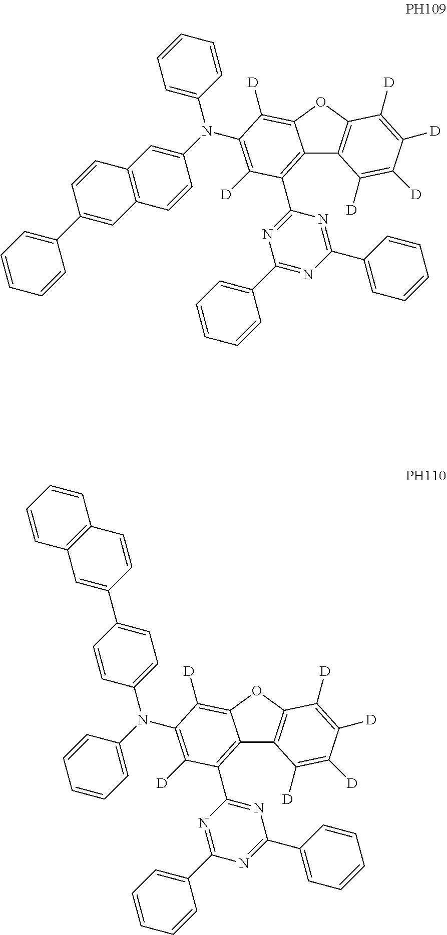 [SMILES: [2H]c1c([2H])c([2H])c2c(oc3c([2H])c(N(c4ccccc4)c4ccc5cc(-c6ccccc6)ccc5c4)c([2H])c(-c4nc(-c5ccccc5)nc(-c5ccccc5)n4)c32)c1[2H] ; [2H]c1c([2H])c([2H])c2c(oc3c([2H])c(N(c4ccccc4)c4ccc(-c5ccc6ccccc6c5)cc4)c([2H])c(-c4nc(-c5ccccc5)nc(-c5ccccc5)n4)c32)c1[2H]]

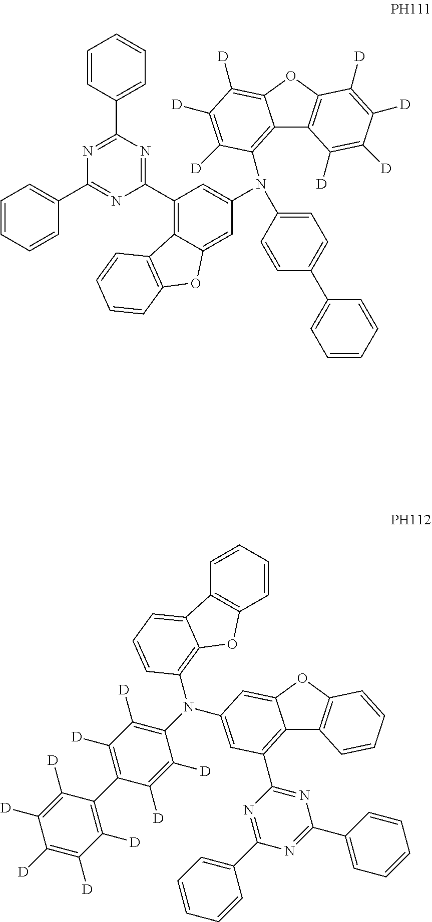 [SMILES: [2H]c1c([2H])c([2H])c2c(oc3c([2H])c([2H])c([2H])c(N(c4ccc(-c5ccccc5)cc4)c4cc(-c5nc(-c6ccccc6)nc(-c6ccccc6)n5)c5c(c4)oc4ccccc45)c32)c1[2H] ; [2H]c1c([2H])c([2H])c(-c2c([2H])c([2H])c(N(c3cc(-c4nc(-c5ccccc5)nc(-c5ccccc5)n4)c4c(c3)oc3ccccc34)c3cccc4c3oc3ccccc34)c([2H])c2[2H])c([2H])c1[2H]]

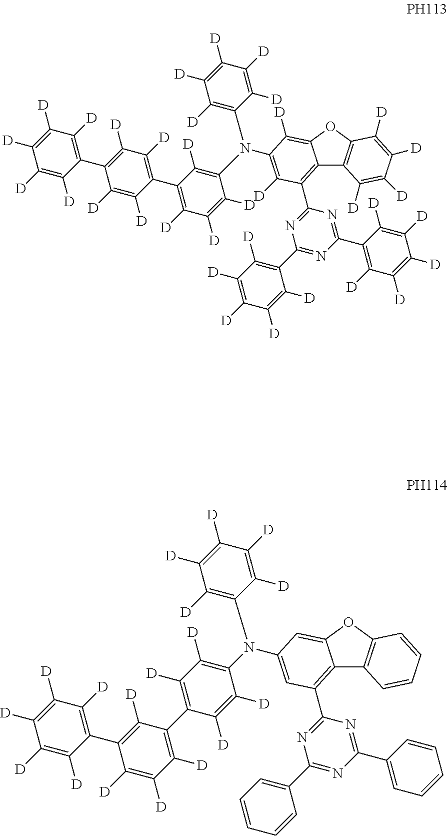 [SMILES: [2H]c1c([2H])c([2H])c(-c2nc(-c3c([2H])c([2H])c([2H])c([2H])c3[2H])nc(-c3c([2H])c(N(c4c([2H])c([2H])c([2H])c([2H])c4[2H])c4c([2H])c([2H])c([2H])c(-c5c([2H])c([2H])c(-c6c([2H])c([2H])c([2H])c([2H])c6[2H])c([2H])c5[2H])c4[2H])c([2H])c4oc5c([2H])c([2H])c([2H])c([2H])c5c34)n2)c([2H])c1[2H] ; [2H]c1c([2H])c([2H])c(-c2c([2H])c([2H])c([2H])c(-c3c([2H])c([2H])c(N(c4cc(-c5nc(-c6ccccc6)nc(-c6ccccc6)n5)c5c(c4)oc4ccccc45)c4c([2H])c([2H])c([2H])c([2H])c4[2H])c([2H])c3[2H])c2[2H])c([2H])c1[2H]]

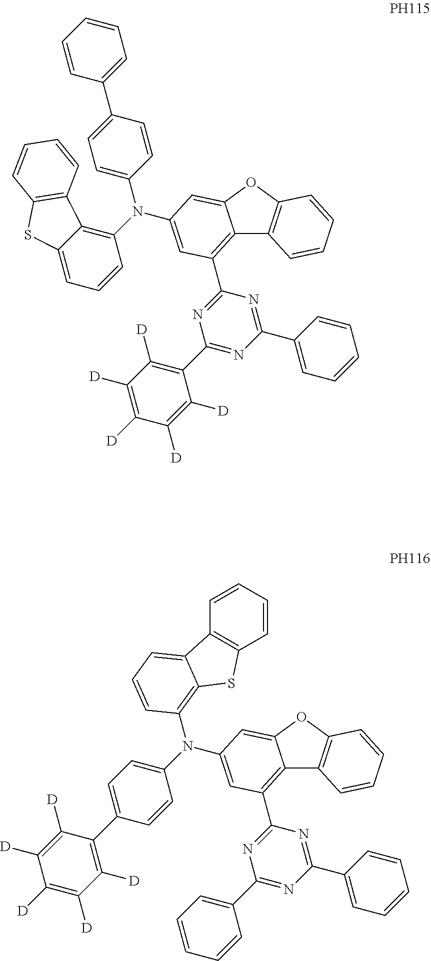 [SMILES: [2H]c1c([2H])c([2H])c(-c2nc(-c3ccccc3)nc(-c3cc(N(c4ccc(-c5ccccc5)cc4)c4cccc5sc6ccccc6c45)cc4oc5ccccc5c34)n2)c([2H])c1[2H] ; [2H]c1c([2H])c([2H])c(-c2ccc(N(c3cc(-c4nc(-c5ccccc5)nc(-c5ccccc5)n4)c4c(c3)oc3ccccc34)c3cccc4c3sc3ccccc34)cc2)c([2H])c1[2H]]

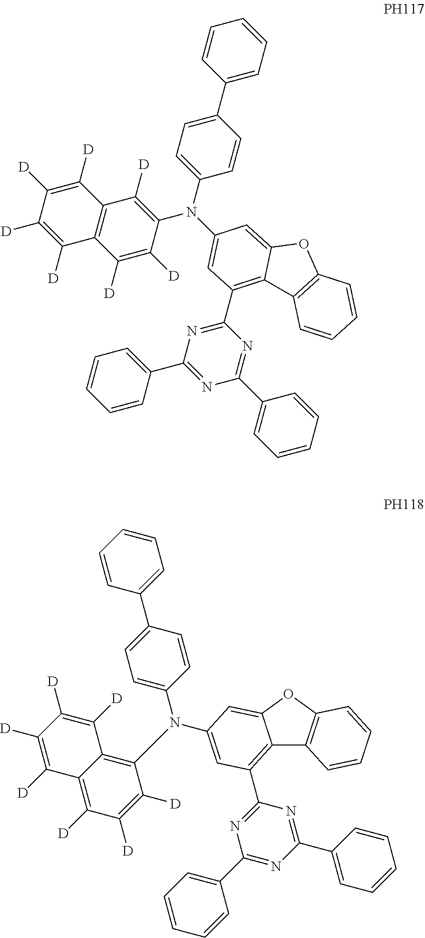 [SMILES: [2H]c1c([2H])c([2H])c2c([2H])c(N(c3ccc(-c4ccccc4)cc3)c3cc(-c4nc(-c5ccccc5)nc(-c5ccccc5)n4)c4c(c3)oc3ccccc34)c([2H])c([2H])c2c1[2H] ; [2H]c1c([2H])c([2H])c2c(N(c3ccc(-c4ccccc4)cc3)c3cc(-c4nc(-c5ccccc5)nc(-c5ccccc5)n4)c4c(c3)oc3ccccc34)c([2H])c([2H])c([2H])c2c1[2H]]

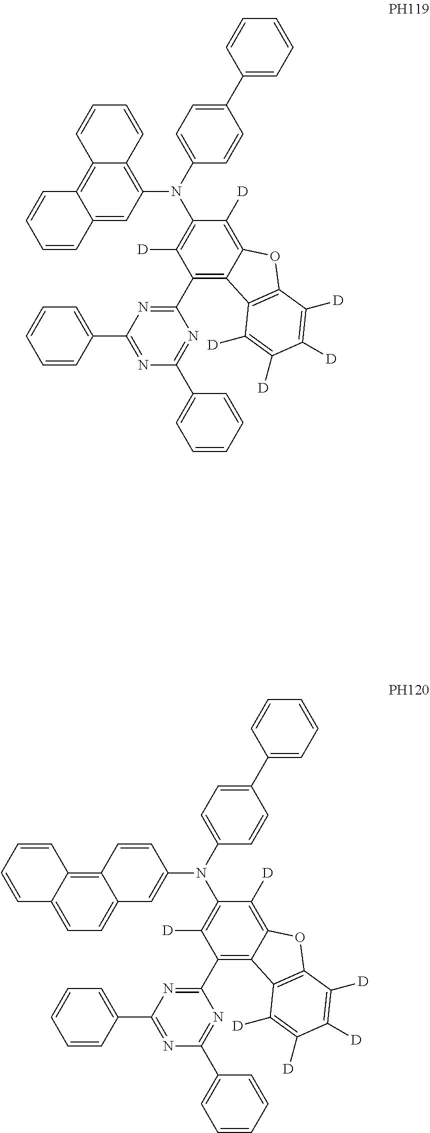 [SMILES: [2H]c1c([2H])c([2H])c2c(oc3c([2H])c(N(c4ccc(-c5ccccc5)cc4)c4cc5ccccc5c5ccccc45)c([2H])c(-c4nc(-c5ccccc5)nc(-c5ccccc5)n4)c32)c1[2H] ; [2H]c1c([2H])c([2H])c2c(oc3c([2H])c(N(c4ccc(-c5ccccc5)cc4)c4ccc5c(ccc6ccccc65)c4)c([2H])c(-c4nc(-c5ccccc5)nc(-c5ccccc5)n4)c32)c1[2H]]

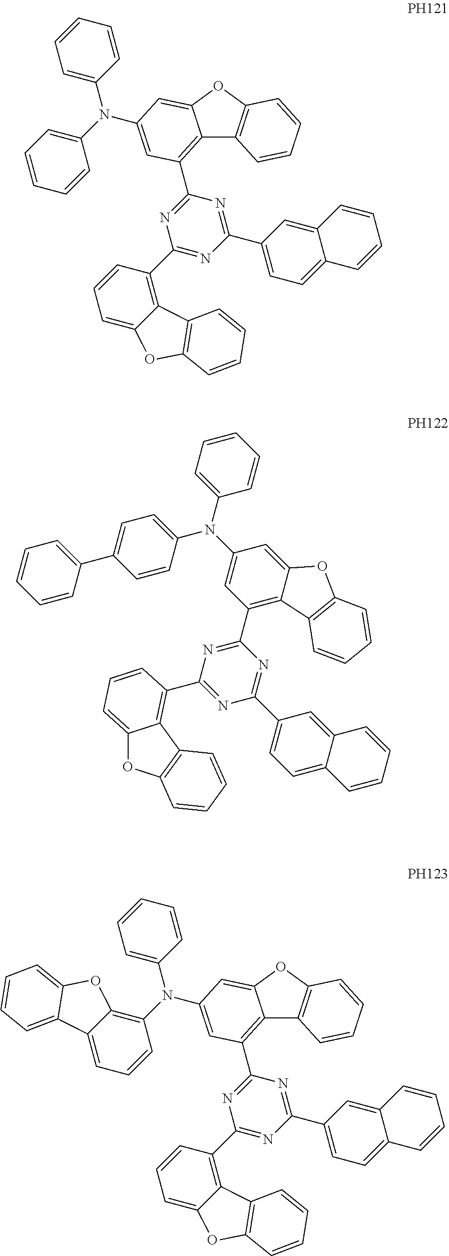 [SMILES: c1ccc(N(c2ccccc2)c2cc(-c3nc(-c4ccc5ccccc5c4)nc(-c4cccc5oc6ccccc6c45)n3)c3c(c2)oc2ccccc23)cc1 ; c1ccc(-c2ccc(N(c3ccccc3)c3cc(-c4nc(-c5ccc6ccccc6c5)nc(-c5cccc6oc7ccccc7c56)n4)c4c(c3)oc3ccccc34)cc2)cc1 ; c1ccc(N(c2cc(-c3nc(-c4ccc5ccccc5c4)nc(-c4cccc5oc6ccccc6c45)n3)c3c(c2)oc2ccccc23)c2cccc3c2oc2ccccc23)cc1]

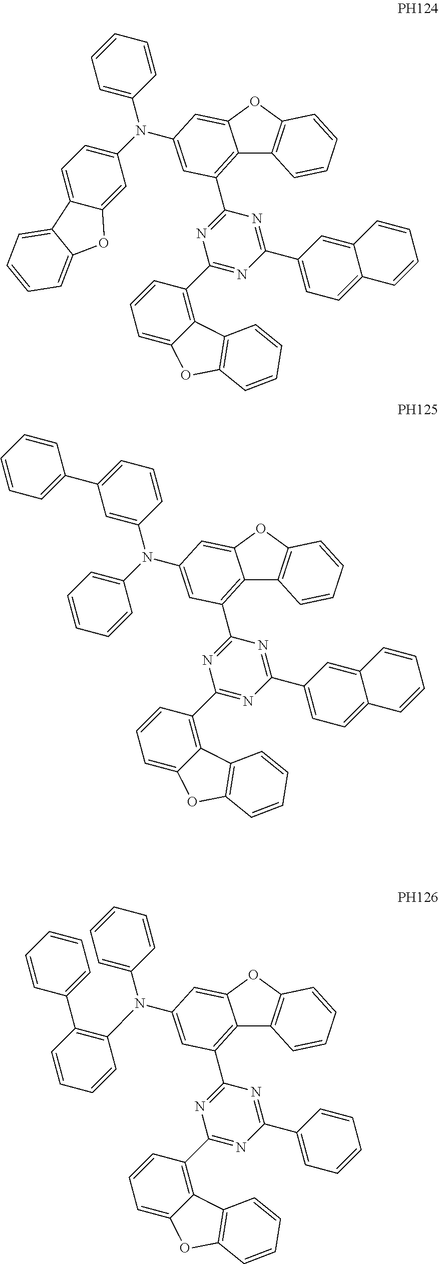 [SMILES: c1ccc(N(c2ccc3c(c2)oc2ccccc23)c2cc(-c3nc(-c4ccc5ccccc5c4)nc(-c4cccc5oc6ccccc6c45)n3)c3c(c2)oc2ccccc23)cc1 ; c1ccc(-c2cccc(N(c3ccccc3)c3cc(-c4nc(-c5ccc6ccccc6c5)nc(-c5cccc6oc7ccccc7c56)n4)c4c(c3)oc3ccccc34)c2)cc1 ; c1ccc(-c2nc(-c3cccc4oc5ccccc5c34)nc(-c3cc(N(c4ccccc4)c4ccccc4-c4ccccc4)cc4oc5ccccc5c34)n2)cc1]

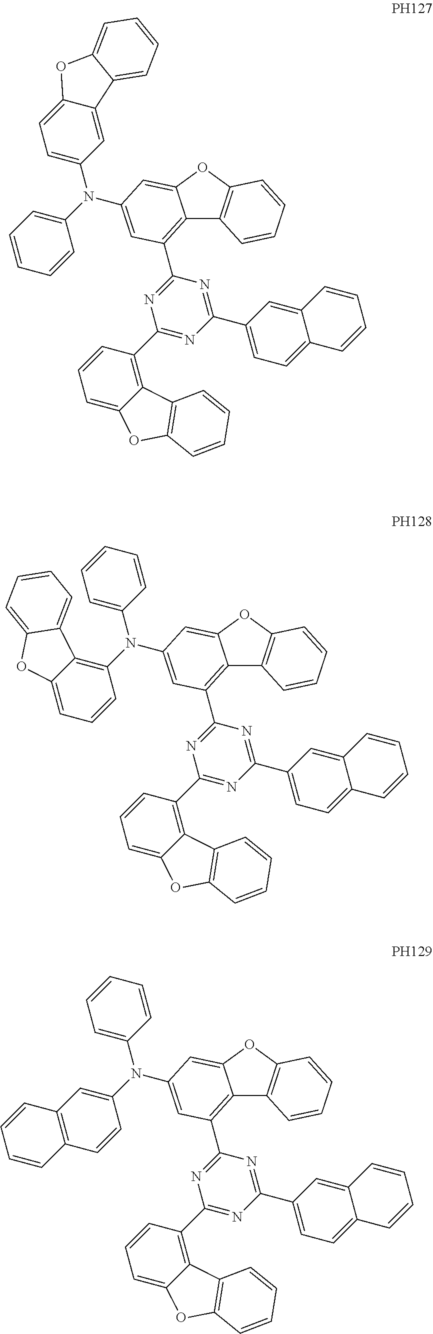 [SMILES: c1ccc(N(c2cc(-c3nc(-c4ccc5ccccc5c4)nc(-c4cccc5oc6ccccc6c45)n3)c3c(c2)oc2ccccc23)c2ccc3oc4ccccc4c3c2)cc1 ; c1ccc(N(c2cc(-c3nc(-c4ccc5ccccc5c4)nc(-c4cccc5oc6ccccc6c45)n3)c3c(c2)oc2ccccc23)c2cccc3oc4ccccc4c23)cc1 ; c1ccc(N(c2ccc3ccccc3c2)c2cc(-c3nc(-c4ccc5ccccc5c4)nc(-c4cccc5oc6ccccc6c45)n3)c3c(c2)oc2ccccc23)cc1]

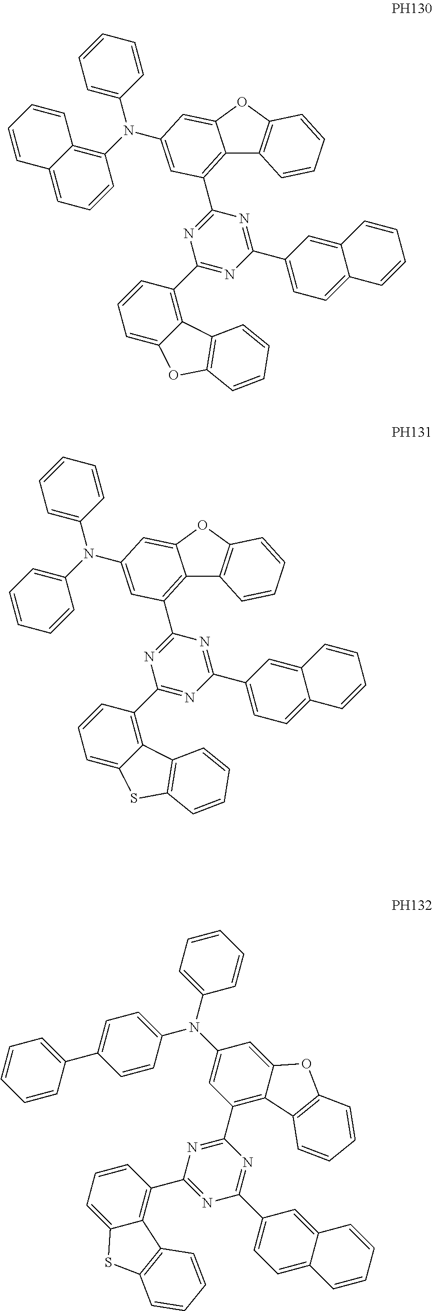 [SMILES: c1ccc(N(c2cc(-c3nc(-c4ccc5ccccc5c4)nc(-c4cccc5oc6ccccc6c45)n3)c3c(c2)oc2ccccc23)c2cccc3ccccc23)cc1 ; c1ccc(N(c2ccccc2)c2cc(-c3nc(-c4ccc5ccccc5c4)nc(-c4cccc5sc6ccccc6c45)n3)c3c(c2)oc2ccccc23)cc1 ; c1ccc(-c2ccc(N(c3ccccc3)c3cc(-c4nc(-c5ccc6ccccc6c5)nc(-c5cccc6sc7ccccc7c56)n4)c4c(c3)oc3ccccc34)cc2)cc1]

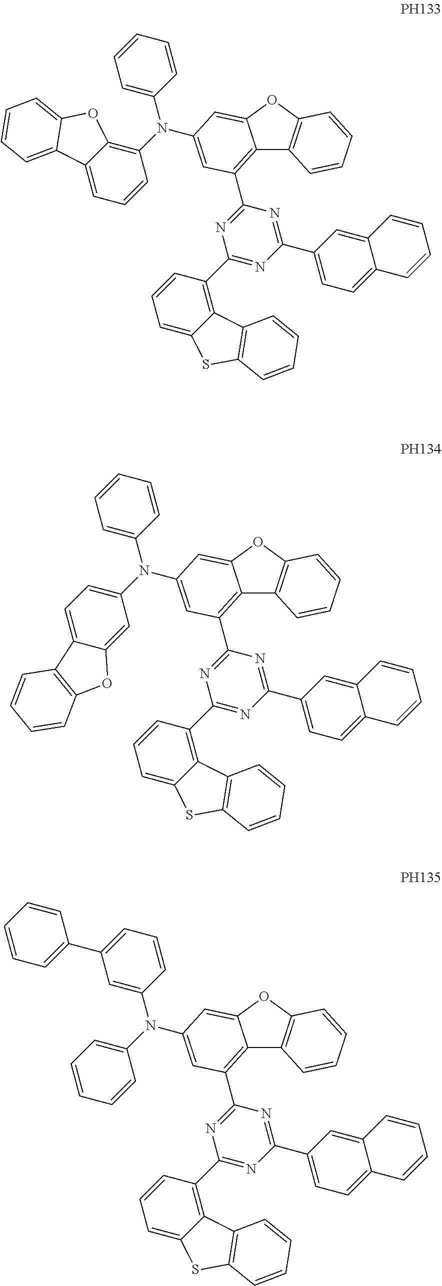 [SMILES: c1ccc(N(c2cc(-c3nc(-c4ccc5ccccc5c4)nc(-c4cccc5sc6ccccc6c45)n3)c3c(c2)oc2ccccc23)c2cccc3c2oc2ccccc23)cc1 ; c1ccc(N(c2ccc3c(c2)oc2ccccc23)c2cc(-c3nc(-c4ccc5ccccc5c4)nc(-c4cccc5sc6ccccc6c45)n3)c3c(c2)oc2ccccc23)cc1 ; c1ccc(-c2cccc(N(c3ccccc3)c3cc(-c4nc(-c5ccc6ccccc6c5)nc(-c5cccc6sc7ccccc7c56)n4)c4c(c3)oc3ccccc34)c2)cc1]

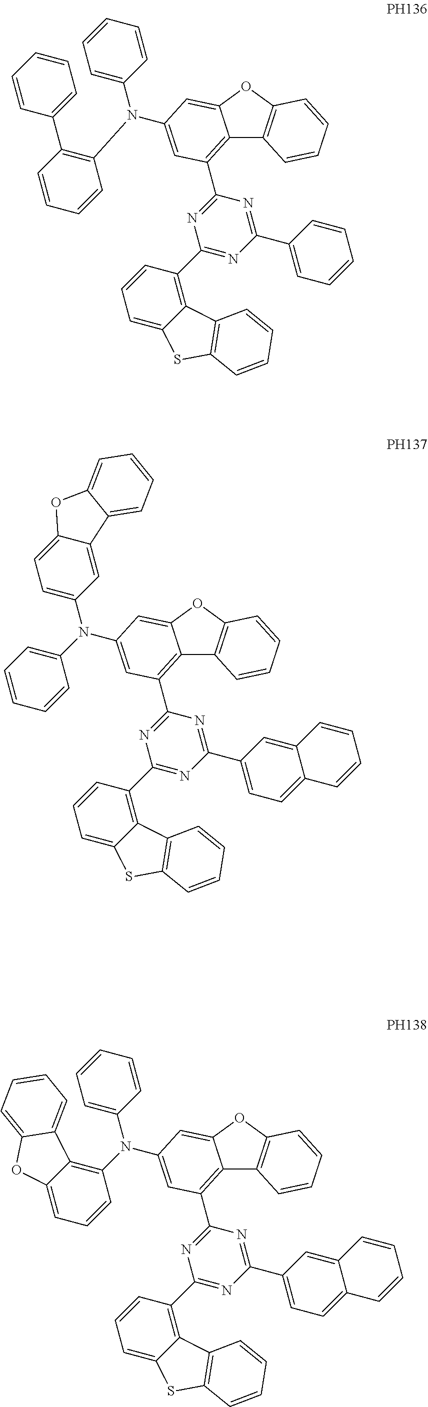 [SMILES: c1ccc(-c2nc(-c3cc(N(c4ccccc4)c4ccccc4-c4ccccc4)cc4oc5ccccc5c34)nc(-c3cccc4sc5ccccc5c34)n2)cc1 ; c1ccc(N(c2cc(-c3nc(-c4ccc5ccccc5c4)nc(-c4cccc5sc6ccccc6c45)n3)c3c(c2)oc2ccccc23)c2ccc3oc4ccccc4c3c2)cc1 ; c1ccc(N(c2cc(-c3nc(-c4ccc5ccccc5c4)nc(-c4cccc5sc6ccccc6c45)n3)c3c(c2)oc2ccccc23)c2cccc3oc4ccccc4c23)cc1]

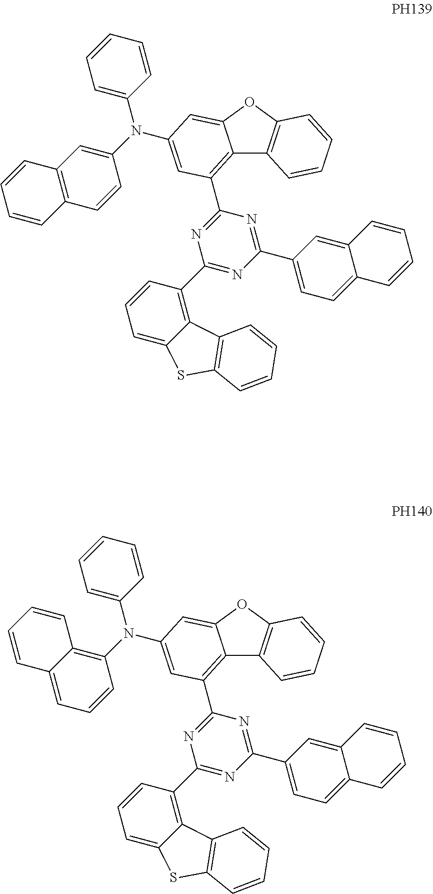 [SMILES: c1ccc(N(c2ccc3ccccc3c2)c2cc(-c3nc(-c4ccc5ccccc5c4)nc(-c4cccc5sc6ccccc6c45)n3)c3c(c2)oc2ccccc23)cc1 ; c1ccc(N(c2cc(-c3nc(-c4ccc5ccccc5c4)nc(-c4cccc5sc6ccccc6c45)n3)c3c(c2)oc2ccccc23)c2cccc3ccccc23)cc1]

A content on the organic light emitting device including the heterocyclic compound represented by Chemical Formula 1 described above may be applied to the organic light emitting device further including the heterocyclic compound represented by Chemical Formula 2.

In another exemplary embodiment, the heterocyclic compound represented by Chemical Formula 2 can be used as a light emitting material for a light emitting layer of the organic light emitting device.

In still another exemplary embodiment, the heterocyclic compound represented by Chemical Formula 2 may be used as a light emitting material fora light emitting layer of an organic light emitting device, and may be used as a p-type host material.

In the organic light emitting device of the present invention, the organic material layer may include the heterocyclic compound represented by Chemical Formula 1 and the heterocyclic compound represented by Chemical Formula 2. The organic material layer may be formed by pre-mixing the heterocyclic compound represented by Chemical Formula 1 and the heterocyclic compound represented by Chemical Formula 2 and using a thermal vacuum deposition method.

In another organic light emitting device, the organic material layer includes a light emitting layer, the light emitting layer includes a host material, and the host material may include the heterocyclic compound represented by Chemical Formula 1 and the heterocyclic compound represented by Chemical Formula 2.

In still another organic light emitting device, the organic material layer includes a light emitting layer, includes the heterocyclic compound represented by Chemical Formula 1 as an n-type host material for the light emitting layer, and may include the heterocyclic compound represented by Chemical Formula 2 as a p-type host material.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by supplying the heterocyclic compound represented by Chemical Formula 1 and the heterocyclic compound represented by Chemical Formula 2 as each individual supply source, and then using a thermal vacuum deposition method.

In an exemplary embodiment of the present application, provided is a method for manufacturing an organic light emitting device, in which the forming of the organic material layer forms the organic material layer by pre-mixing the heterocyclic compound represented by Chemical Formula 1 and the heterocyclic compound represented by Chemical Formula 2, and using a thermal vacuum deposition method.

The organic light emitting device according to an exemplary embodiment of the present application may be manufactured by typical manufacturing methods and materials of the organic light emitting device, except that the above-described heterocyclic compound is used to form an organic material layer.

The organic light emitting device of the present invention may further include one or two or more layers selected from the group consisting of a light emitting layer, a hole injection layer, a hole transport layer, an electron blocking layer, an electron injection layer, an electron transport layer, a hole auxiliary layer, and a hole blocking layer.

In an exemplary embodiment of the present application, provided is a composition for an organic material layer of an organic light emitting device, which includes the heterocyclic compound represented by Chemical Formula 1 and the heterocyclic compound represented by Chemical Formula 2.

The weight ratio of the heterocyclic compound represented by Chemical Formula 1: the heterocyclic compound represented by Chemical Formula 2 in the composition may be 1:10 to 10:1, 1:8 to 8:1, 1:5 to 5:1, and 1:2 to 2:1, but is not limited thereto.

The composition may be used when an organic material layer of an organic light emitting device is formed, and particularly, may be more preferably used as a host material for the light emitting layer.

The composition is in a form in which two or more compounds are simply mixed, materials in a powder state may also be mixed before an organic material layer of an organic light emitting device is formed, and it is possible to mix compounds in a liquid state at a temperature which is equal to or more than a suitable temperature. The composition is in a solid state at a temperature which is equal to or less than the melting point of each material, and may be maintained as a liquid phase when the temperature is adjusted.

The composition may additionally include materials publicly known in the art such as solvents and additives.

Hereinafter, the present specification will be described in more detail through Examples, but these Examples are provided only for exemplifying the present application, and are not intended to limit the scope of the present application.

### SYNTHESIS EXAMPLES

### [Preparation Example 1] Preparation of Compound 3

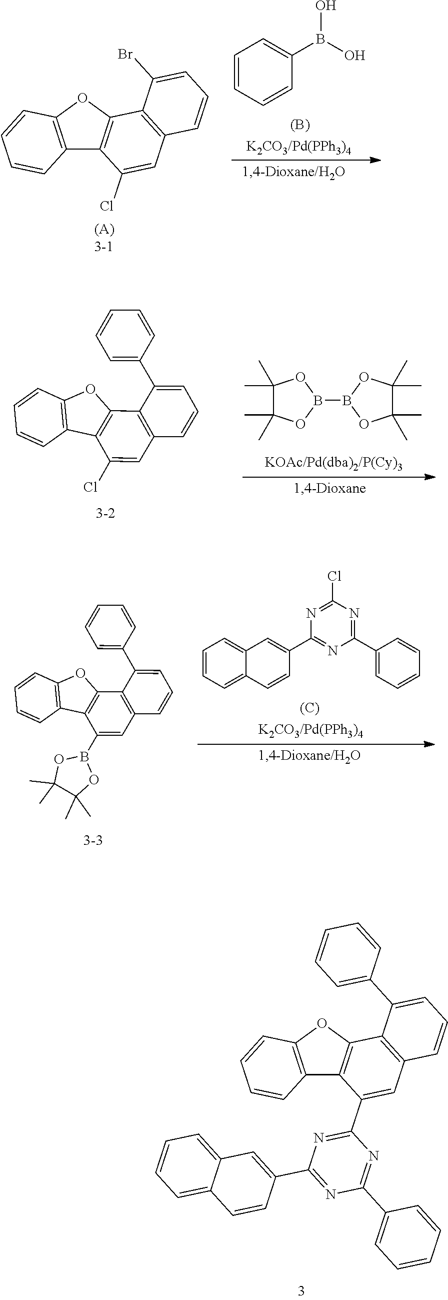

### 1) Preparation of Compound 3-2

1,4-Dioxane (300 ml) and H₂O (60 ml) were put into a compound 1-bromo-6-chloronaphtho[1,2-b]benzofuran (A) (15 g, 0.045 mol, 1 eq), phenylboronic acid (B) (6.6 g, 0.054 mol, 1.2 eq), K₂CO₃ (18.8 g, 0.136 mol, 3 eq), and Pd(PPh₃)₄ (2.6 g, 0.002 mol, 0.05 eq), and the resulting mixture was stirred at 100° C. for 8 hours. After the reaction was terminated by adding water, extraction was performed using methylene chloride (MC) and water. Thereafter, moisture was removed with MgSO₄. The residue was separated by silica gel column to obtain 12 g of Compound 3-2 with a yield of 81%.

### 2) Preparation of Compound 3-3

1,4-Dioxane (120 ml) was put into Compound 3-2 (12 g, 0.036 mol, 1 eq) 4,4,4′,4′,5,5,5′,5′-octamethyl-2,2′-bi(1,3,2-dioxaborolane) (13.9 g, 0.055 mol, 1.5 eq), KOAc (10.73 g, 0.109 mol, 3 eq), Pd(dba)₂ (1 g, 0.002 mol, 0.05 eq), and P(Cy)₃ (1 g, 0.004 mol, 0.1 eq), and the resulting mixture was stirred at 100° C. for 9 hours. After the reaction was terminated by adding water thereto, extraction was performed using methylene chloride (MC) and water. Thereafter, moisture was removed with MgSO₄. The mixture was separated by silica gel column to obtain 10 g of Compound 3-3 with a yield of 65%.

### 3) Preparation of Compound 3

1,4-Dioxane (200 ml) and H₂O (50 ml) were put into Compound 3-3 (10 g, 0.024 mol, 1 eq), 2-chloro-4-(naphthalen-2-yl)-6-phenyl-1,3,5-triazine (C) (8.3 g, 0.026 mol, 1.1 eq), K₂CO₃ (9.8 g, 0.071 mol, 3 eq), and Pd(PPh₃)₄ (1.4 g, 0.001 mol, 0.05 eq), and the resulting mixture was stirred at 100° C. for 7 hours. After the reaction was terminated by adding water, extraction was performed using methylene chloride (MC) and water. Thereafter, moisture was removed with MgSO₄. The residue was separated by silica gel column to obtain 9 g of Compound 3 with a yield of 64%.

### [Preparation Example 2] Preparation of Compound PH4

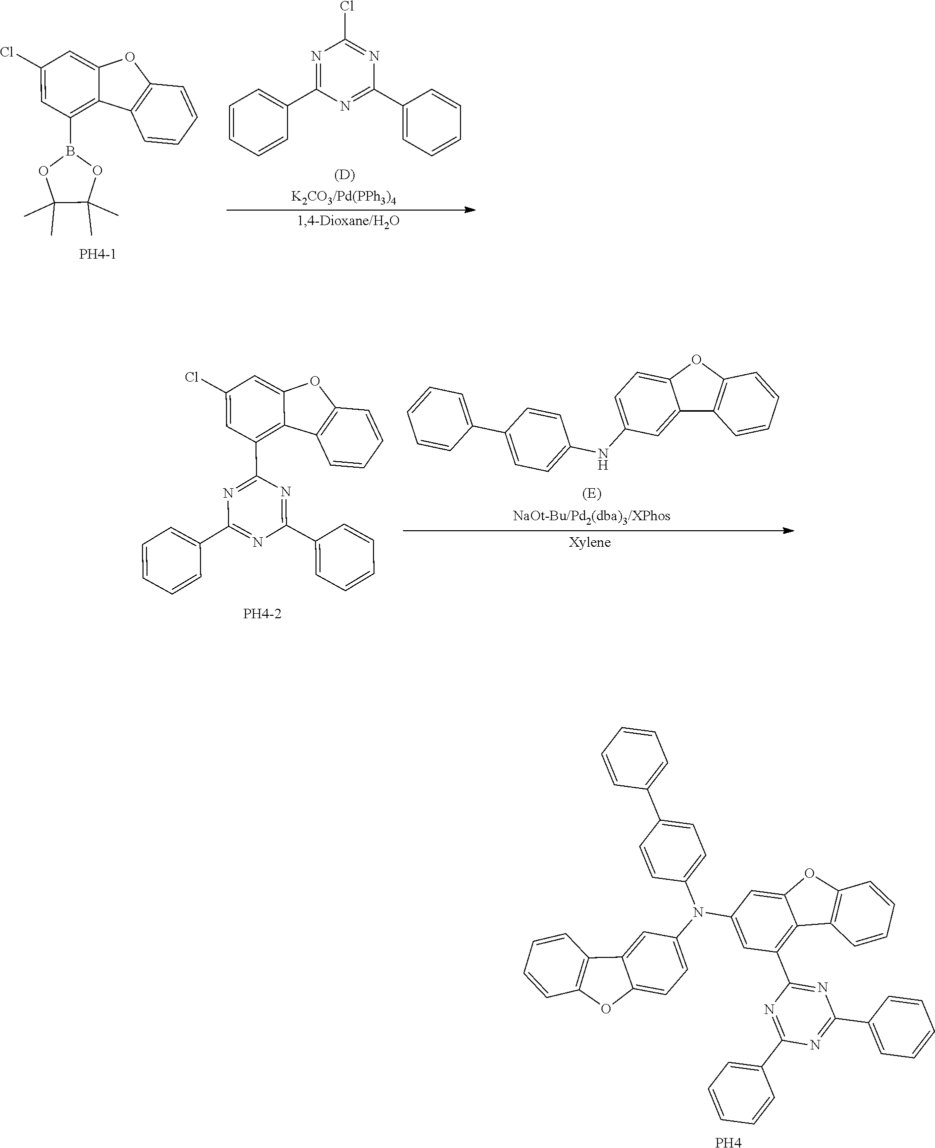

### 1) Preparation of Compound PH4-2

1,4-Dioxane (300 ml) and H₂O (60 ml) were put into PH4-1 (10 g, 0.030 mol, 1 eq), 2-chloro-4,6-diphenyl-1,3,5-triazine (D) (8.9 g, 0.033 mol, 1.1 eq), K₂CO₃ (12.6 g, 0.091 mol, 3 eq), and Pd(PPh₃)₄ (1.4 g, 0.0015 mol, 0.05 eq), and the resulting mixture was stirred at 100° C. for 8 hours. After the reaction was terminated by adding water, extraction was performed using methylene chloride (MC) and water. Thereafter, moisture was removed with MgSO₄. The residue was separated by silica gel column to obtain 9 g of Compound PH4-2 with a yield of 68%.

### 2) Preparation of Compound PH4

Xylene (180 ml) was put into Compound PH4-2 (9 g, 0.021 mol, 1 eq), N-([1,1′-biphenyl]-4-yl)dibenzo[b,d]furan-3-amine (E) (7.7 g, 0.023 mol, 1.1 eq), NaOt-Bu (4 g, 0.041 mol, 2 eq), Pd₂(dba)₃ (0.9 g, 0.001 mol, 0.05 eq), and X Phos (1 g, 0.002 mol, 0.1 eq), and the resulting mixture was stirred at 100° C. for 9 hours. After the reaction was terminated by adding water, extraction was performed using methylene chloride (MC) and water. Thereafter, moisture was removed with MgSO₄. The residue was separated by silica gel column to obtain 9 g of Compound PH4 with a yield of 59%.

Compounds were synthesized in the same manner as in the preparation of Preparation Examples 1 and 2, except that Intermediates A, B, C, D, and E of the following Table 1 were used instead of (A), (B), and (C) in Preparation Example 1 and (D) and (E) in Preparation Example 2.

TABLE 1

| Com- | | | |
|---|---|---|---|
| pound | | | |
| No. | Intermediate A | Intermediate B | Intermediate C |
| 26 | 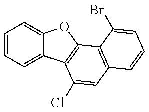 [SMILES: Clc1cc2cccc(Br)c2c2oc3ccccc3c12] | 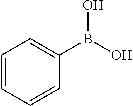 | 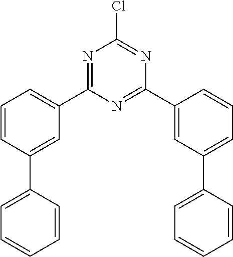 [SMILES: Clc1nc(-c2cccc(-c3ccccc3)c2)nc(-c2cccc(-c3ccccc3)c2)n1] |
| 45 | 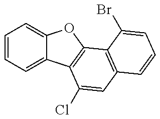 [SMILES: Clc1cc2cccc(Br)c2c2oc3ccccc3c12] | 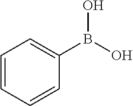 | 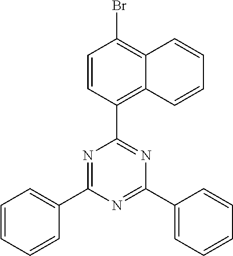 [SMILES: Brc1ccc(-c2nc(-c3ccccc3)nc(-c3ccccc3)n2)c2ccccc12] |
| 67 | 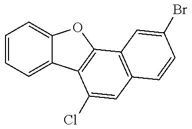 [SMILES: Clc1cc2ccc(Br)cc2c2oc3ccccc3c12] | 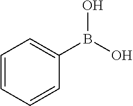 | 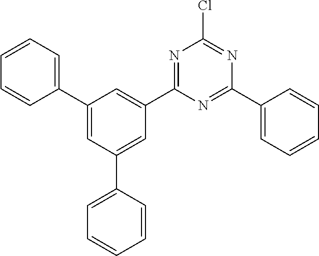 [SMILES: Clc1nc(-c2ccccc2)nc(-c2cc(-c3ccccc3)cc(-c3ccccc3)c2)n1] |
| 88 | 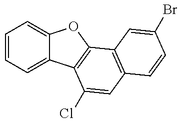 [SMILES: Clc1cc2ccc(Br)cc2c2oc3ccccc3c12] | 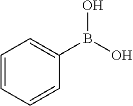 | 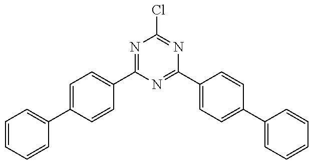 [SMILES: Clc1nc(-c2ccc(-c3ccccc3)cc2)nc(-c2ccc(-c3ccccc3)cc2)n1] |
| 101 | 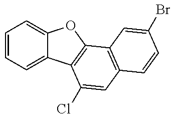 [SMILES: Clc1cc2ccc(Br)cc2c2oc3ccccc3c12] | 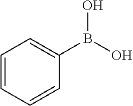 | 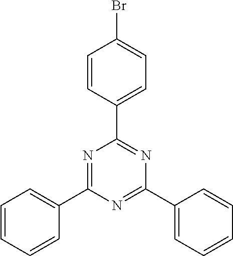 [SMILES: Brc1ccc(-c2nc(-c3ccccc3)nc(-c3ccccc3)n2)cc1] |
| 129 | 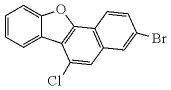 [SMILES: Clc1cc2cc(Br)ccc2c2oc3ccccc3c12] | 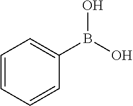 | 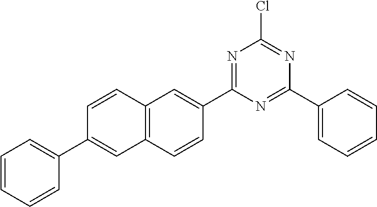 [SMILES: Clc1nc(-c2ccccc2)nc(-c2ccc3cc(-c4ccccc4)ccc3c2)n1] |
| 143 | 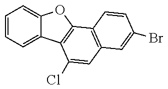 [SMILES: Clc1cc2cc(Br)ccc2c2oc3ccccc3c12] | 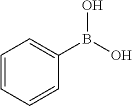 | 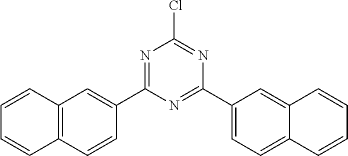 [SMILES: Clc1nc(-c2ccc3ccccc3c2)nc(-c2ccc3ccccc3c2)n1] |
| 175 | 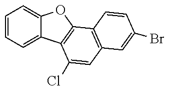 [SMILES: Clc1cc2cc(Br)ccc2c2oc3ccccc3c12] | 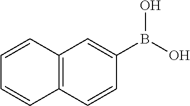 [SMILES: OB(O)c1ccc2ccccc2c1] | 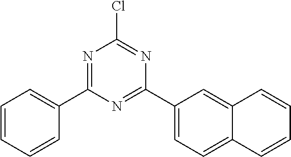 [SMILES: Clc1nc(-c2ccccc2)nc(-c2ccc3ccccc3c2)n1] |
| 188 | 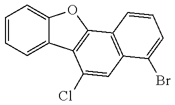 [SMILES: Clc1cc2c(Br)cccc2c2oc3ccccc3c12] | 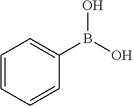 | 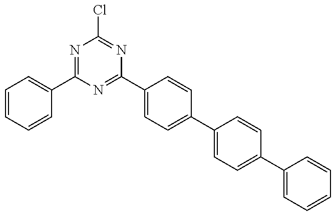 [SMILES: Clc1nc(-c2ccccc2)nc(-c2ccc(-c3ccc(-c4ccccc4)cc3)cc2)n1] |
| 213 | 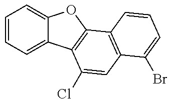 [SMILES: Clc1cc2c(Br)cccc2c2oc3ccccc3c12] | 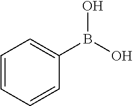 | 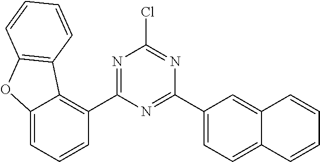 [SMILES: Clc1nc(-c2ccc3ccccc3c2)nc(-c2cccc3oc4ccccc4c23)n1] |
| 232 | 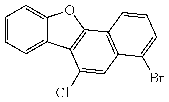 [SMILES: Clc1cc2c(Br)cccc2c2oc3ccccc3c12] | 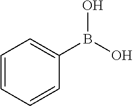 | 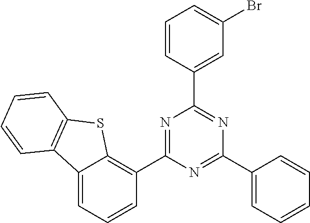 [SMILES: Brc1cccc(-c2nc(-c3ccccc3)nc(-c3cccc4c3sc3ccccc34)n2)c1] |
| 244 | 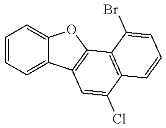 [SMILES: Clc1cc2c3ccccc3oc2c2c(Br)cccc12] | 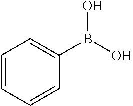 | 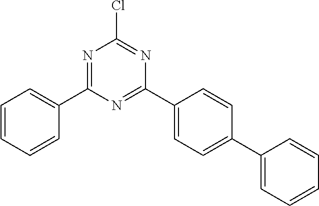 [SMILES: Clc1nc(-c2ccccc2)nc(-c2ccc(-c3ccccc3)cc2)n1] |
| 277 | 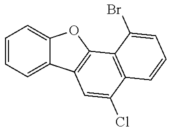 [SMILES: Clc1cc2c3ccccc3oc2c2c(Br)cccc12] | 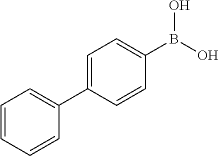 [SMILES: OB(O)c1ccc(-c2ccccc2)cc1] | 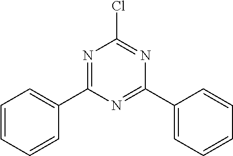 [SMILES: Clc1nc(-c2ccccc2)nc(-c2ccccc2)n1] |
| 300 | 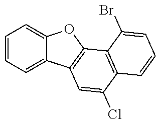 [SMILES: Clc1cc2c3ccccc3oc2c2c(Br)cccc12] | 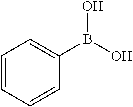 | 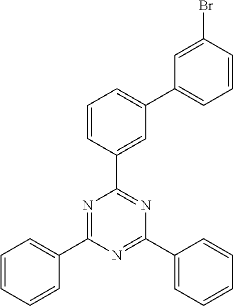 [SMILES: Brc1cccc(-c2cccc(-c3nc(-c4ccccc4)nc(-c4ccccc4)n3)c2)c1] |
| 310 | 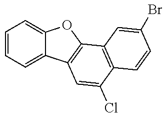 [SMILES: Clc1cc2c3ccccc3oc2c2cc(Br)ccc12] | 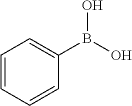 | 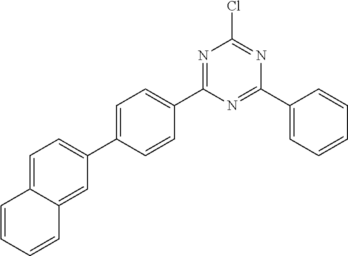 [SMILES: Clc1nc(-c2ccccc2)nc(-c2ccc(-c3ccc4ccccc4c3)cc2)n1] |
| 334 | 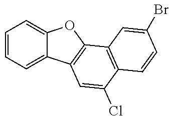 [SMILES: Clc1cc2c3ccccc3oc2c2cc(Br)ccc12] | 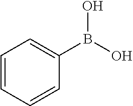 | 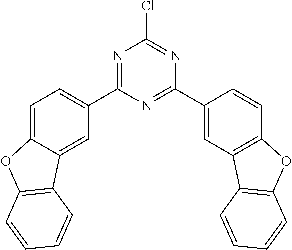 [SMILES: Clc1nc(-c2ccc3oc4ccccc4c3c2)nc(-c2ccc3oc4ccccc4c3c2)n1] |
| 353 | 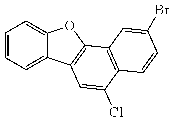 [SMILES: Clc1cc2c3ccccc3oc2c2cc(Br)ccc12] | 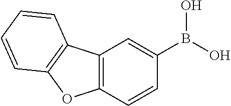 [SMILES: OB(O)c1ccc2oc3ccccc3c2c1] | 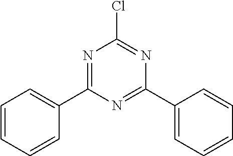 [SMILES: Clc1nc(-c2ccccc2)nc(-c2ccccc2)n1] |
| 372 | 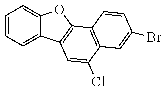 [SMILES: Clc1cc2c3ccccc3oc2c2ccc(Br)cc12] | 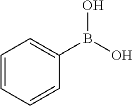 | 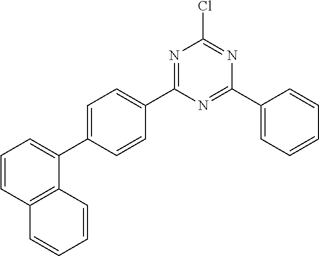 [SMILES: Clc1nc(-c2ccccc2)nc(-c2ccc(-c3cccc4ccccc34)cc2)n1] |
| 400 | 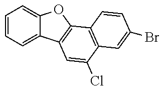 [SMILES: Clc1cc2c3ccccc3oc2c2ccc(Br)cc12] | 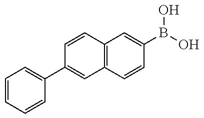 [SMILES: OB(O)c1ccc2cc(-c3ccccc3)ccc2c1] | 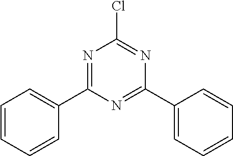 [SMILES: Clc1nc(-c2ccccc2)nc(-c2ccccc2)n1] |
| 402 | 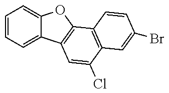 [SMILES: Clc1cc2c3ccccc3oc2c2ccc(Br)cc12] | 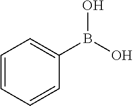 | 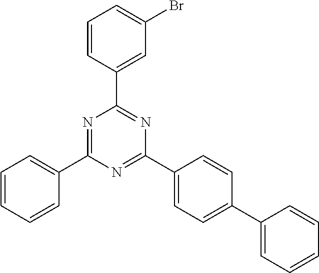 [SMILES: Brc1cccc(-c2nc(-c3ccccc3)nc(-c3ccc(-c4ccccc4)cc3)n2)c1] |
| 440 | 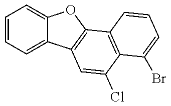 [SMILES: Clc1cc2c3ccccc3oc2c2cccc(Br)c12] | 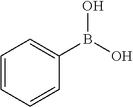 | 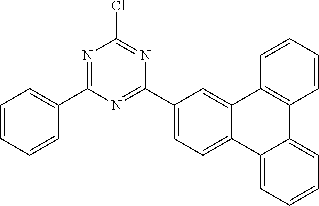 [SMILES: Clc1nc(-c2ccccc2)nc(-c2ccc3c4ccccc4c4ccccc4c3c2)n1] |
| 443 | 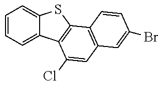 [SMILES: Clc1cc2cc(Br)ccc2c2sc3ccccc3c12] | 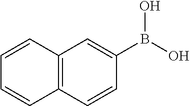 [SMILES: OB(O)c1ccc2ccccc2c1] | 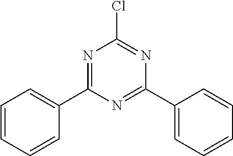 [SMILES: Clc1nc(-c2ccccc2)nc(-c2ccccc2)n1] |
| 466 | 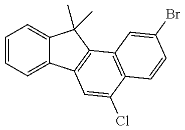 [SMILES: CC1(C)c2ccccc2-c2cc(Cl)c3ccc(Br)cc3c21] | 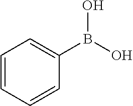 | 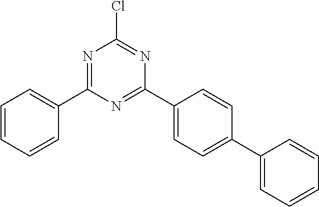 [SMILES: Clc1nc(-c2ccccc2)nc(-c2ccc(-c3ccccc3)cc2)n1] |

| Compound | | |
|---|---|---|
| No. | Intermediate D | Intermediate E |
| PH30 | 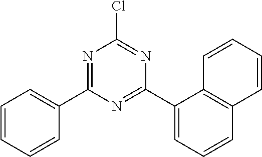 [SMILES: Clc1nc(-c2ccccc2)nc(-c2cccc3ccccc23)n1] | 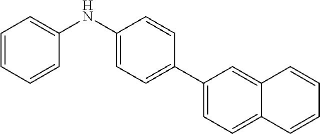 [SMILES: c1ccc(Nc2ccc(-c3ccc4ccccc4c3)cc2)cc1] |
| PH57 | 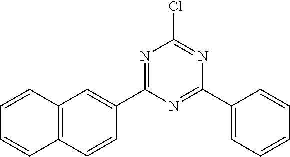 [SMILES: Clc1nc(-c2ccccc2)nc(-c2ccc3ccccc3c2)n1] | 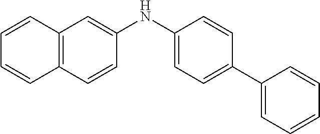 [SMILES: c1ccc(-c2ccc(Nc3ccc4ccccc4c3)cc2)cc1] |
| PH67 | 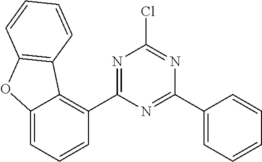 [SMILES: Clc1nc(-c2ccccc2)nc(-c2cccc3oc4ccccc4c23)n1] | 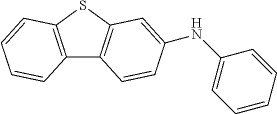 [SMILES: c1ccc(Nc2ccc3c(c2)sc2ccccc23)cc1] |
| PH100 | 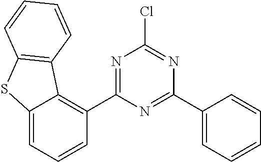 [SMILES: Clc1nc(-c2ccccc2)nc(-c2cccc3sc4ccccc4c23)n1] | 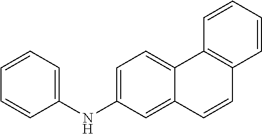 [SMILES: c1ccc(Nc2ccc3c(ccc4ccccc43)c2)cc1] |
| PH106 | 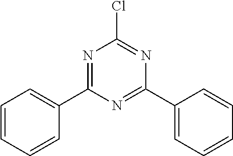 [SMILES: Clc1nc(-c2ccccc2)nc(-c2ccccc2)n1] | 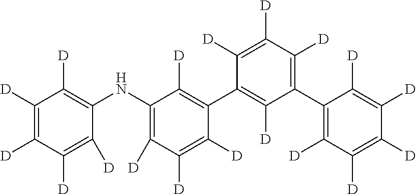 [SMILES: [2H]c1c([2H])c([2H])c(Nc2c([2H])c([2H])c([2H])c(-c3c([2H])c([2H])c([2H])c(-c4c([2H])c([2H])c([2H])c([2H])c4[2H])c3[2H])c2[2H])c([2H])c1[2H]] |
| PH122 | 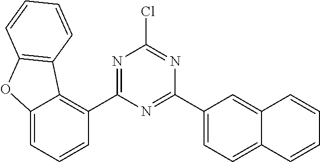 [SMILES: Clc1nc(-c2ccc3ccccc3c2)nc(-c2cccc3oc4ccccc4c23)n1] | 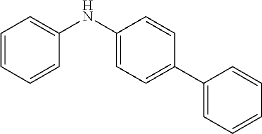 [SMILES: c1ccc(Nc2ccc(-c3ccccc3)cc2)cc1] |

### [Preparation Example 3] Preparation of Compound 557

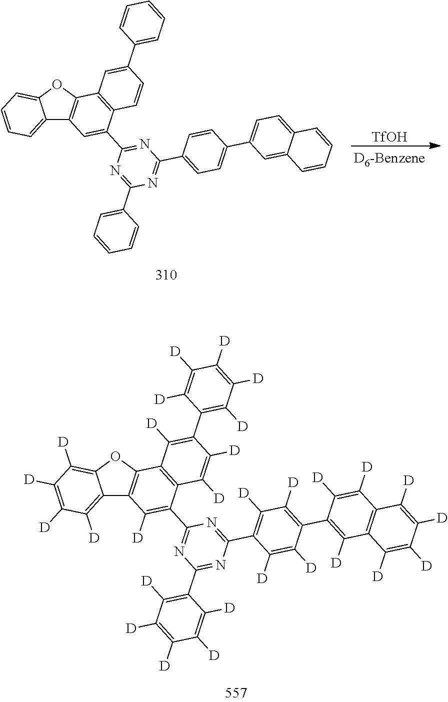

After Compound 310 (7 g, 0.011 mol, 1 eq) prepared in Preparation Example 1, triflic acid (TfOH) (2.4 g, 0.016 mol, 1.5 eq), and D₆-benzene) (70 ml) were put into a flask, the resulting mixture was stirred at 100° C. for 7 hours. After the reaction was terminated by adding water thereto, extraction was performed using methylene chloride (MC) and water. Thereafter, moisture was removed with MgSO₄. The residue was separated by silica gel column to obtain 6 g of Compound 557 with a yield of 82%.

Compounds were prepared in the same manner as in the Preparation Examples, and the synthesis confirmation results thereof are shown in Tables 2 and 3. Table 2 shows the measured values of ¹H NMR (CDCl₃, 200 MHz), and Table 3 shows the measured values of field desorption mass spectrometry (FD-MS).

TABLE 2

| Compound | ¹H NMR(CDCl₃, 200M Hz) |
|---|---|
| 3 | δ = 9.09(1H, s), 8.49(2H, m), 8.28(2H, m), 7.89(5H, m), |
| | 7.79(2H, m), 7.66~7.32(13H, m) |
| 26 | δ = 8.55(2H, m), 8.42(1H, d), 8.08(8H, m), 7.79(2H, m), |
| | 7.66(10H, m), 7.35(4H, m) |
| 45 | δ = 8.55(2H, m), 8.42(1H, d), 8.28(4H, m), 8.01(3H, m), |
| | 7.89(1H, d), 7.79(2H, m), 7.66~7.32(16H, m) |
| 67 | δ = 8.34(1H, s), 8.28(2H, m), 7.92(2H, m), 7.73(1H, d), |
| | 7.66(4H, m), 7.52~7.38(21H, m) |
| 88 | δ = 8.34(1H, s), 7.85(6H, m), 7.73(1H, d), 7.66(1H, d), |
| | 7.52~7.25(22H, m) |
| 101 | δ = 8.28(5H, m), 7.85(4H, m), 7.73(2H, m), |
| | 7.51~7.25(16H, m) |
| 129 | δ = 9.09(1H, s), 8.49(1H, d), 8.28(2H, m), 8.06(1H, d), |
| | 7.89(3H, m), 7.73(3H, m), 7.58~7.32(18H, m) |
| 143 | δ = 9.09(2H, m), 8.49(2H, m), 8.00(8H, m), |
| | 7.73~7.32(15H, m) |
| 175 | δ = 9.09(1H, s), 8.49(1H, d), 8.28(2H, m), 8.00(8H, m), |
| | 7.58(11H, m), 7.35(4H, m) |
| 188 | δ = 8.42(1H, d), 8.28(2H, m), 8.14(1H, d), 7.79(5H, m), |
| | 7.66~7.25(22H, m) |
| 213 | δ = 8.42(1H, d), 8.14(1H, d), 7.89(7H, m), |
| | 7.66~7.25(20H, m) |
| 232 | δ = 8.45(3H, m), 8.24(3H, m), 8.14(1H, d), 7.98(1H, d), |
| | 7.89(1H, d), 7.79(3H, m), 7.70~7.32(17H, m) |
| 244 | δ = 8.51(1H, d), 8.41(1H, d), 8.28(2H, m), 7.85(5H, m), |
| | 7.71~7.25(18H, m) |
| 277 | δ = 8.51(1H, d), 8.41(1H, d), 8.28(4H, m), 7.89(1H, d), |
| | 7.71~7.25(20H, m) |
| 300 | δ = 8.51(1H, d), 8.41(1H, d), 8.24(5H, m), 7.89(1H, d), |
| | 7.79(2H, m), 7.71~7.32(21H, m) |
| 310 | δ = 8.28(2H, m), 8.14(1H, d), 7.85(8H, m), |
| | 7.73~7.25(18H, m) |
| 334 | δ = 8.14(1H, d), 7.81(7H, m), 7.66(8H, m), |
| | 7.52~7.32(11H, m) |
| 353 | δ = 8.28(4H, m), 8.14(1H, d), 7.95(5H, m), 7.71(5H, m), |
| | 7.51~7.32(10H, m) |
| 372 | δ = 8.55(1H, d), 8.42(1H, d), 8.28(2H, m), 8.14(3H, m), |
| | 7.85(5H, m) 7.66~7.24(17H, m) |
| 400 | δ = 8.28(4H, m), 8.14(1H, d), 7.92(5H, m), 7.71(4H, m), |
| | 7.58~7.32(15H. m) |
| 402 | δ = 8.24(3H, m), 8.14(1H, d), 7.89(5H, m), 7.70(3H, m), |
| | 7.57~7.32(19H, m) |
| 440 | δ = 9.15(1H, s), 8.93(2H, m), 8.42(1H, d), 8.28(2H, m), |
| | 8.12(5H, m), 7.89~7.32(18H, m) |
| 443 | δ = 8.45(1H, d), 7.28(4H, m), 7.98(5H, m), 7.73(2H, m), |
| | 7.58(13H, m) |
| 466 | δ = 8.28(2H, m), 8.11(2H, m), 7.99(1H, s), 7.85(3H, m), |
| | 7.63(2H, m), 7.51~7.41(14H, m), 7.24(3H, m), |
| | 1.85(6H, s) |
| 557 | δ = No NMR data |
| PH4 | δ = 8.28(4H, m), 7.89(2H, m), 7.66(3H, m), |
| | 7.54~7.32(20H, m), 6.69(2H, m), 6.39(1H, d) |
| PH30 | δ = 8.55(1H, d), 8.28(2H, m), 8.04(7H, m), |
| | 7.66~7.32(17H, m), 7.20(2H, m), 6.81(1H, t), 6.63(4H, m) |
| PH57 | δ = 9.09(1H, s), 8.49(1H, d), 8.28(2H, m), 8.00(2H, m), |
| | 7.84(6H, m), 7.66~7.36(20H, m), 6.69(2H, m) |
| PH67 | δ = 8.45(1H, d), 8.28(2H, m), 7.98(1H, d), 7.89(2H, m), |
| | 7.75(2H, m), 7.66(3H, m), 7.52~7.32(12H, m), |
| | 7.20(2H, m), 7.06(1H, s), 6.88(2H, m), 6.63(2H, m) |
| PH100 | δ = 8.93(1H, d), 8.68(1H, d), 8.45(1H, d), 8.28(2H, m), |
| | 8.12(1H, d), 7.94(6H, m), 7.71(3H, m), 7.39(11H, m), |
| | 7.20(2H, m), 7.08(1H, d), 6.81(1H, t), 6.63(2H, m) |
| PH106 | δ = 8.28(4H, m), 7.89(1H, d), 7.66(1H, d), |
| | 7.51~7.38(10H, m) |
| PH122 | δ = 9.09(1H, s), 8.49(1H, d), 8.00(5H, m), 7.75(1H, d), |
| | 7.66~7.32(19H, m), 7.20(2H, m), 6.81(1H, t), 6.63(4H, m) |

TABLE 3

| Compound | FD-MS |
|---|---|
| 3 | m/z = 575.20 |
| 26 | m/z = 625.22 |
| 45 | m/z = 651.23 |
| 67 | m/z = 677.25 |
| 88 | m/z = 677.25 |
| 101 | m/z = 601.22 |
| 129 | m/z = 651.23 |
| 143 | m/z = 625.22 |
| 175 | m/z = 625.22 |
| 188 | m/z = 677.25 |
| 213 | m/z = 691.23 |
| 232 | m/z = 707.20 |
| 244 | m/z = 601.22 |
| 277 | m/z = 601.22 |
| 300 | m/z = 677.25 |
| 310 | m/z = 651.23 |
| 334 | m/z = 705.21 |
| 353 | m/z = 615.19 |
| 372 | m/z = 651.23 |
| 400 | m/z = 651.23 |
| 402 | m/z = 677.25 |
| 440 | m/z = 675.23 |
| 443 | m/z = 591.18 |
| 466 | m/z = 627.27 |
| 557 | m/z = 680.41 |
| PH4 | m/z = 732.25 |
| PH30 | m/z = 742.27 |
| PH57 | m/z = 742.27 |
| PH67 | m/z = 762.21 |
| PH100 | m/z = 772.23 |
| PH106 | m/z = 736.39 |
| PH122 | m/z = 782.27 |

### EXPERIMENTAL EXAMPLES

### Experimental Example 1

### (1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA) and a hole transport layer N,N′-di(1-naphthyl)-N,N′-diphenyl-(1,1′-biphenyl)-4,4′-diamine (NPB), which are common layers, were formed on the ITO transparent electrode (positive electrode).

A light emitting layer was thermally vacuum deposited thereon as follows. The light emitting layer was deposited to have a thickness of 400 Å by depositing the compound described in the following Table 4 as a red host from one supply source and doping the host with an Ir compound at 3 wt % using (piq)₂(Ir)_{(acac)} as a red phosphorescent dopant. Thereafter, bathophenanthroline (Bphen) as a hole blocking layer was deposited to have a thickness of 30 Å, and Alq₃ as an electron transport layer was deposited to have a thickness of 250 Å thereon. Finally, lithium fluoride (LiF) was deposited to have a thickness of 10 Å on the electron transport layer to form an electron injection layer, and then an aluminum (AI) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁸ to 10⁻⁶ torr for each material, and used for the manufacture of OLED.

For the organic electroluminescence device manufactured as described above, electroluminescence (EL) characteristics were measured by M 7000 manufactured by McScience Inc., and based on the measurement result thereof, T90 was measured by a service life measurement equipment (M 6000) manufactured by McScience Inc., when the reference luminance was 6,000 cd/m². The results of measuring the driving voltage, light emitting efficiency, color coordinate (CIE) and service life of the organic light emitting device manufactured according to the present invention are shown in Table 4.

TABLE 4

| | | | Light | | |
|---|---|---|---|---|---|
| | | Driving | emitting | | Service |
| | | voltage | efficiency | CIE | life |
| No. | Compound | (V) | (cd/A) | (x, y) | (T90) |
| Example 1 | 3 | 5.1 | 51.6 | (0.68, 0.32) | 63 |
| Example 2 | 26 | 5.0 | 51.4 | (0.68, 0.32) | 67 |
| Example 3 | 45 | 5.1 | 53.2 | (0.68, 0.32) | 71 |
| Example 4 | 67 | 5.0 | 51.7 | (0.68, 0.32) | 70 |
| Example 5 | 88 | 5.0 | 52.3 | (0.68, 0.32) | 63 |
| Example 6 | 101 | 5.0 | 52.0 | (0.68, 0.32) | 64 |
| Example 7 | 129 | 5.1 | 53.4 | (0.68, 0.32) | 66 |
| Example 8 | 143 | 5.0 | 53.3 | (0.68, 0.32) | 70 |
| Example 9 | 175 | 4.9 | 54.0 | (0.68, 0.32) | 81 |
| Example 10 | 188 | 5.1 | 52.0 | (0.68, 0.32) | 65 |
| Example 11 | 213 | 5.1 | 52.8 | (0.68, 0.32) | 69 |
| Example 12 | 232 | 5.0 | 51.7 | (0.68, 0.32) | 73 |
| Example 13 | 244 | 5.0 | 52.1 | (0.68, 0.32) | 65 |
| Example 14 | 277 | 4.9 | 54.2 | (0.68, 0.32) | 82 |
| Example 15 | 300 | 5.1 | 51.3 | (0.68, 0.32) | 72 |
| Example 16 | 310 | 5.1 | 51.5 | (0.68, 0.32) | 65 |
| Example 17 | 334 | 5.0 | 52.1 | (0.68, 0.32) | 72 |
| Example 18 | 353 | 4.9 | 54.1 | (0.68, 0.32) | 80 |
| Example 19 | 372 | 5.0 | 52.7 | (0.68, 0.32) | 67 |
| Example 20 | 400 | 4.9 | 54.2 | (0.68, 0.32) | 83 |
| Example 21 | 402 | 5.0 | 51.4 | (0.68, 0.32) | 72 |
| Example 22 | 440 | 5.1 | 53.1 | (0.68, 0.32) | 65 |
| Example 23 | 443 | 5.3 | 48.5 | (0.68, 0.32) | 54 |
| Example 24 | 466 | 5.4 | 47.7 | (0.68, 0.32) | 53 |
| Example 25 | 557 | 5.0 | 51.5 | (0.68, 0.32) | 70 |
| Comparative | H1 | 5.7 | 44.2 | (0.68, 0.32) | 42 |
| Example 1 | | | | | |
| Comparative | H2 | 5.7 | 44.5 | (0.68, 0.32) | 41 |
| Example 2 | | | | | |
| Comparative | H3 | 5.9 | 41.3 | (0.68, 0.32) | 44 |
| Example 3 | | | | | |
| Comparative | H4 | 5.9 | 41.9 | (0.68, 0.32) | 44 |
| Example 4 | | | | | |
| Comparative | H5 | 5.8 | 42.7 | (0.68, 0.32) | 41 |
| Example 5 | | | | | |
| Comparative | H6 | 5.8 | 42.2 | (0.68, 0.32) | 42 |
| Example 6 | | | | | |
| Comparative | H7 | 5.7 | 43.5 | (0.68, 0.32) | 43 |
| Example 7 | | | | | |
| Comparative | H8 | 5.8 | 42.9 | (0.68, 0.32) | 41 |
| Example 8 | | | | | |

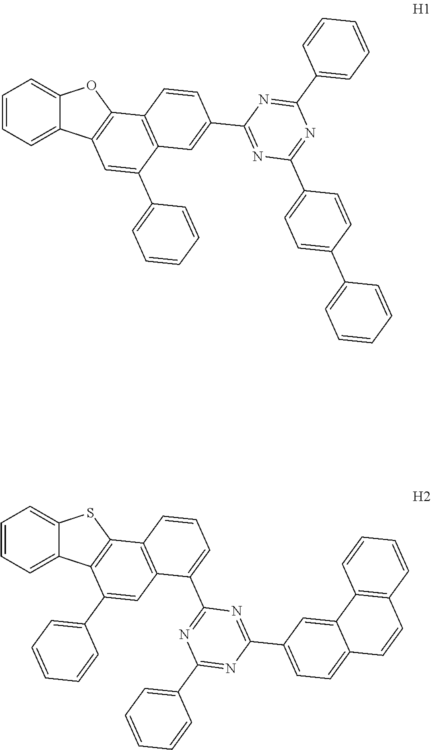 [SMILES: c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2nc(-c3ccc4ccc5ccccc5c4c3)nc(-c3cccc4c3cc(-c3ccccc3)c3c5ccccc5sc43)n2)cc1]

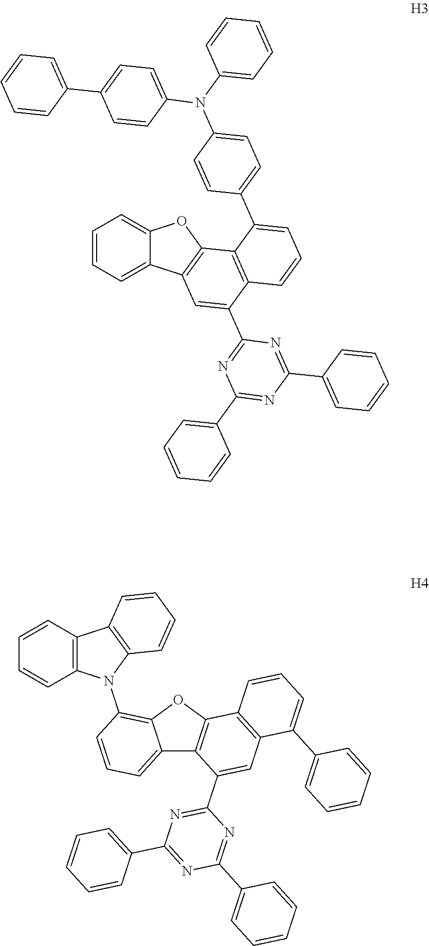 [SMILES: c1ccc(-c2ccc(N(c3ccccc3)c3ccc(-c4cccc5c(-c6nc(-c7ccccc7)nc(-c7ccccc7)n6)cc6c7ccccc7oc6c45)cc3)cc2)cc1 ; c1ccc(-c2nc(-c3ccccc3)nc(-c3cc4c(-c5ccccc5)cccc4c4oc5c(-n6c7ccccc7c7ccccc76)cccc5c34)n2)cc1]

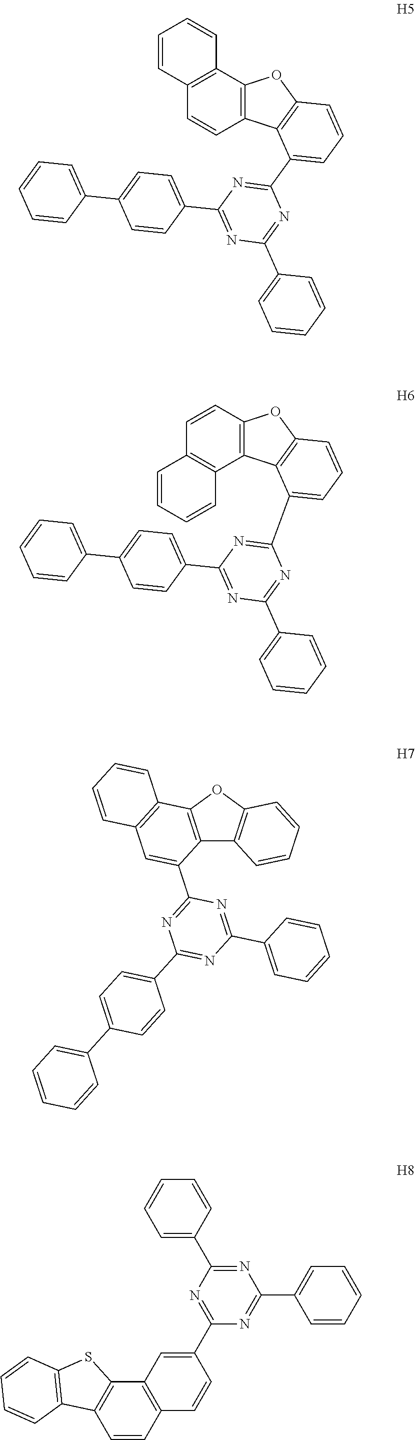 [SMILES: c1ccc(-c2ccc(-c3nc(-c4ccccc4)nc(-c4cccc5oc6c7ccccc7ccc6c45)n3)cc2)cc1 ; c1ccc(-c2ccc(-c3nc(-c4ccccc4)nc(-c4cccc5oc6ccc7ccccc7c6c45)n3)cc2)cc1 ; c1ccc(-c2ccc(-c3nc(-c4ccccc4)nc(-c4cc5ccccc5c5oc6ccccc6c45)n3)cc2)cc1 ; c1ccc(-c2nc(-c3ccccc3)nc(-c3ccc4ccc5c6ccccc6sc5c4c3)n2)cc1]

As can be seen from the results in Table 4, it could be confirmed that the organic light emitting device using a host material of the present invention has a low driving voltage and remarkably improved light emitting efficiency and service life compared to the Comparative Examples.

The heterocyclic compound according to the present application has a molecular weight and a band gap suitable for use in the light emitting layer of the organic light emitting device while having high thermal stability.

A suitable molecular weight facilitates the formation of the light emitting layer of the organic light emitting device, and a suitable band gap prevents the electrons and holes of the light emitting layer from flowing out to help form an effective recombination zone.

In addition, a heterocyclic compound having transfer properties of electrons substituted at an appropriate position solves a hole blocking phenomenon caused by a dopant more than a compound substituted at other positions, and as can be seen from the above device evaluation, it could be confirmed that the compound of the present invention were excellent in all aspects of driving, efficiency and service life compared to the Comparative Examples.

### Experimental Example 2

### (1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA), a hole transport layer N,N′-di(1-naphthyl)-N,N′-diphenyl-(1,1′-biphenyl)-4,4′-diamine (NPB) and an electron blocking layer cyclohexylidenebis[N,N-bis(4-methylphenyl)benzenamine](TAPC), which are common layers, were formed on the ITO transparent electrode (positive electrode). A light emitting layer was thermally vacuum deposited thereon as follows. The light emitting layer was deposited to have a thickness of 400 Å by depositing two types of the compounds described in the following Table 5 as a red host from a single supply source and doping the host with an Ir compound at 3 wt % using (piq)₂(Ir)_{(acac)} as a red phosphorescent dopant. Thereafter, Bphen as a hole blocking layer was deposited to have a thickness of 30 Å, and 2,2′,2″-(1,3,5-benzinetriyl)-tris(1-phenyl-1-H-benzimidazole) (TPBI) as an electron transport layer was deposited to have a thickness of 250 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁸ to 10⁻⁶ torr for each material, and used for the manufacture of OLED. For the organic electroluminescence device manufactured as described above, electroluminescence (EL) characteristics were measured by M 7000 manufactured by McScience Inc., and based on the measurement result thereof, T90 was measured by a service life measurement equipment (M 6000) manufactured by McScience Inc., when the reference luminance was 6,000 cd/m². The results of measuring the driving voltage, light emitting efficiency, color coordinate (CIE) and service life of the organic light emitting device manufactured according to the present invention are shown in Table 5.

TABLE 5

| | | | | Light | | |
|---|---|---|---|---|---|---|
| | | | Driving | emitting | | Service |
| | | Ratio | voltage | efficiency | CIE | life |
| No. | Compound | (P/N) | (V) | (cd/A) | (x, y) | (T90) |
| Example 26 | PH4:3 | 1:3 | 4.7 | 52.6 | (0.68, 0.32) | 154 |
| Example 27 | PH4:3 | 1:2 | 4.7 | 51.1 | (0.68, 0.32) | 158 |
| Example 28 | PH4:3 | 1:1 | 4.6 | 52.7 | (0.68, 0.32) | 172 |
| Example 29 | PH4:3 | 2:1 | 4.8 | 52.5 | (0.68, 0.32) | 156 |
| Example 30 | PH4:3 | 3:1 | 4.7 | 53.4 | (0.68, 0.32) | 154 |
| Example 31 | PH4:26 | 1:1 | 4.7 | 52.3 | (0.68, 0.32) | 177 |
| Example 32 | PH4:45 | 1:1 | 4.8 | 52.4 | (0.68, 0.32) | 165 |
| Example 33 | PH4:67 | 1:1 | 4.7 | 51.8 | (0.68, 0.32) | 177 |
| Example 34 | PH30:88 | 1:1 | 4.6 | 51.6 | (0.68, 0.32) | 161 |
| Example 35 | PH30:101 | 1:1 | 4.6 | 52.6 | (0.68, 0.32) | 158 |
| Example 36 | PH30:129 | 1:1 | 4.7 | 51.5 | (0.68, 0.32) | 162 |
| Example 37 | PH30:143 | 1:1 | 4.6 | 51.2 | (0.68, 0.32) | 170 |
| Example 38 | PH57:175 | 1:1 | 4.5 | 54.0 | (0.68, 0.32) | 185 |
| Example 39 | PH57:188 | 1:1 | 4.7 | 52.4 | (0.68, 0.32) | 172 |
| Example 40 | PH57:213 | 1:1 | 4.8 | 51.6 | (0.68, 0.32) | 174 |
| Example 41 | PH57:232 | 1:1 | 4.8 | 51.0 | (0.68, 0.32) | 152 |
| Example 42 | PH67:244 | 1:1 | 4.7 | 52.3 | (0.68, 0.32) | 166 |
| Example 43 | PH67:277 | 1:1 | 4.5 | 54.2 | (0.68, 0.32) | 186 |
| Example 44 | PH67:300 | 1:1 | 4.8 | 51.6 | (0.68, 0.32) | 164 |
| Example 45 | PH67:310 | 1:1 | 4.6 | 52.7 | (0.68, 0.32) | 160 |
| Example 46 | PH100:334 | 1:1 | 4.7 | 53.2 | (0.68, 0.32) | 176 |
| Example 47 | PH100:353 | 1:1 | 4.5 | 54.3 | (0.68, 0.32) | 184 |
| Example 48 | PH100:372 | 1:1 | 4.6 | 52.5 | (0.68, 0.32) | 173 |
| Example 49 | PH100:400 | 1:1 | 4.5 | 54.0 | (0.68, 0.32) | 183 |
| Example 50 | PH106:402 | 1:1 | 4.8 | 51.8 | (0.68, 0.32) | 152 |
| Example 51 | PH106:440 | 1:1 | 4.7 | 52.6 | (0.68, 0.32) | 169 |
| Example 52 | PH106:443 | 1:1 | 5.0 | 48.5 | (0.68, 0.32) | 124 |
| Example 53 | PH106:466 | 1:1 | 5.1 | 47.7 | (0.68, 0.32) | 128 |
| Example 54 | PH30:557 | 1:1 | 4.7 | 52.1 | (0.68, 0.32) | 177 |
| Comparative | PH30:H1 | 1:1 | 5.4 | 45.2 | (0.68, 0.32) | 93 |
| Example 9 | | | | | | |
| Comparative | PH57:H2 | 1:1 | 5.4 | 45.7 | (0.68, 0.32) | 97 |
| Example 10 | | | | | | |
| Comparative | PH57:H3 | 1:1 | 5.5 | 44.1 | (0.68, 0.32) | 105 |
| Example 11 | | | | | | |
| Comparative | PH67:H4 | 1:1 | 5.5 | 44.4 | (0.68, 0.32) | 108 |
| Example 12 | | | | | | |
| Comparative | PH67:H5 | 1:1 | 5.5 | 45.2 | (0.68, 0.32) | 94 |
| Example 13 | | | | | | |
| Comparative | PH100:H6 | 1:1 | 5.5 | 45.3 | (0.68, 0.32) | 90 |
| Example 14 | | | | | | |
| Comparative | PH100:H7 | 1:1 | 5.4 | 44.8 | (0.68, 0.32) | 88 |
| Example 15 | | | | | | |
| Comparative | PH106:H8 | 1:1 | 5.5 | 44.9 | (0.68, 0.32) | 98 |
| Example 16 | | | | | | |

As can be seen from the results in Table 5, it could be confirmed that when the heterocyclic compound of the present invention was used as an N-type host and mixed with a P-type host and deposited, the driving, efficiency, and service life of the organic light emitting device were improved.

When a donor (p-host) with good hole transporting ability and an acceptor (n-host) with good electron transporting ability are used as hosts of the light emitting layer, the charge balance within the device can be adjusted because holes are injected into the p-host and electrons are injected into the n-host due to the exciplex phenomenon of the N+P compound. Therefore, it could be seen that a combination of an N-type host compound with appropriate electron transfer properties and a P-type host compound with appropriate hole transfer properties in an appropriate ratio helps improve driving efficiency and service life.

1. A heterocyclic compound represented by the following Chemical Formula 1: 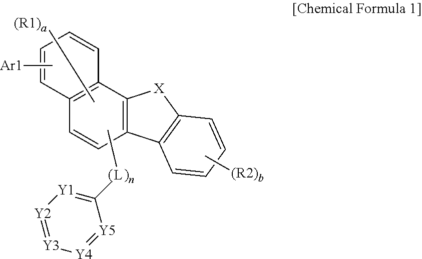
wherein, in Chemical Formula 1,
X is O, S or —C(Ra)(Rb), and Ra and Rb are the same as or different from each other, and are each independently a substituted or unsubstituted C1 to C60 alkyl group or a substituted or unsubstituted C6 to C60 aryl group,
L is a direct bond; or a substituted or unsubstituted C6 to C60 arylene group,
n is an integer from 1 to 3, and when n is 2 or higher, substituents in the parenthesis are the same as or different from each other,
Y1 is CR11 or N, Y2 is CR12 or N, Y3 is CR13 or N, Y4 is CR14 or N, Y5 is CR15 or N, and at least one of Y1 to Y5 is N,
R11 to R15 are the same as or different from each other, and are each independently hydrogen; deuterium; a substituted or unsubstituted C6 to C60 aryl group; or a substituted or unsubstituted C2 to C60 heteroaryl group, or two or more adjacent groups are bonded to each other to form a substituted or unsubstituted C2 to C60 hetero ring,
Ar1 is a substituted or unsubstituted C6 to C60 aryl group; or a substituted or unsubstituted C2 to C60 heteroaryl group,
R1 and R2 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or two or more adjacent groups are bonded to each other to form a substituted or unsubstituted aliphatic or aromatic hydrocarbon ring or hetero ring,
a is an integer from 0 to 4, and when a is 2 or higher, substituents in the parenthesis are the same as or different from each other, and
b is an integer from 0 to 4, and when b is 2 or higher, substituents in the parenthesis are the same as or different from each other.

2. The heterocyclic compound of claim 1, wherein Chemical Formula 1 is represented by any one of the following Chemical Formulae 1-1 and 1-2: 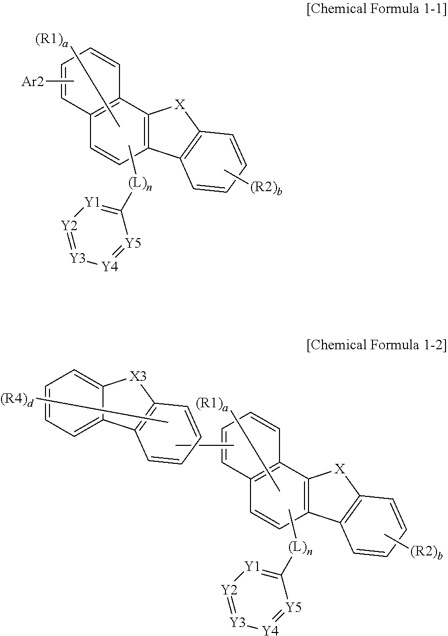
in Chemical Formulae 1-1 and 1-2,
the definitions of X, R1 and R2, L, Y1 to Y5, a, b, and n are the same as the definitions in Chemical Formula 1,
X3 is O or S,
Ar2 is a substituted or unsubstituted C6 to C60 aryl group,
R4 is each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or two or more adjacent groups are bonded to each other to form a substituted or unsubstituted aliphatic or aromatic hydrocarbon ring or hetero ring, and
d is an integer from 0 to 7, and when d is 2 or higher, substituents in the parenthesis are the same as or different from each other.

3. The heterocyclic compound of claim 1, wherein Ar1 is a substituted or unsubstituted C6 to C20 aryl group; or a substituted or unsubstituted C2 to C20 heteroaryl group.

4. The heterocyclic compound of claim 1, wherein L is a direct bond; or a substituted or unsubstituted C6 to C20 arylene group.

5. The heterocyclic compound of claim 1, wherein Chemical Formula 1 is represented by any one of the following Chemical Formulae 1-3 to 1-6: 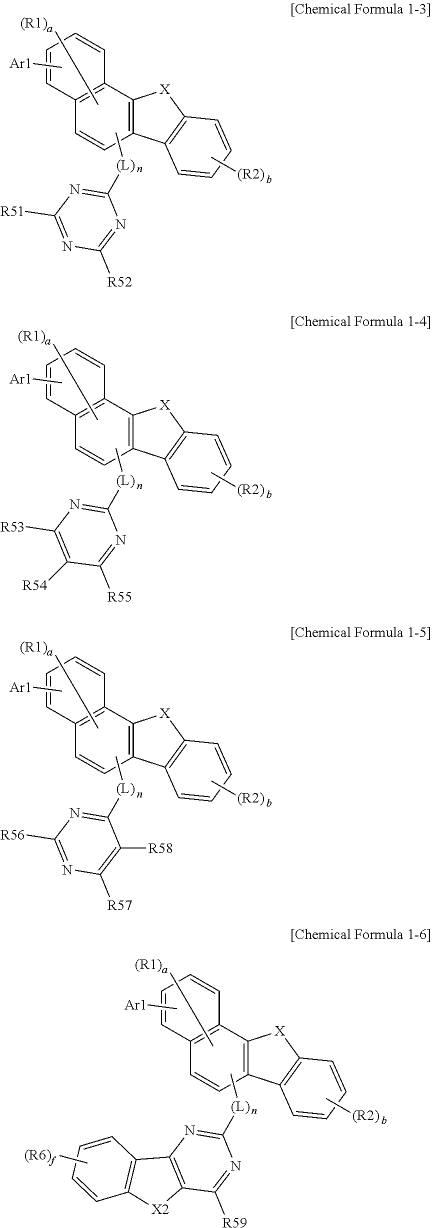
in Chemical Formulae 1-3 to 1-6,
the definitions of X, Ar1, R1, R2, L, a, b, and n are the same as the definitions in Chemical Formula 1,
X2 is O or S,
R51 to R59 are the same as or different from each other, and are each independently hydrogen; deuterium; a substituted or unsubstituted C6 to C60 aryl group; or a substituted or unsubstituted C2 to C60 heteroaryl group,
R6 is hydrogen; deuterium; a substituted or unsubstituted C6 to C60 aryl group; or a substituted or unsubstituted C2 to C60 heteroaryl group, and
f is an integer from 0 to 4, and when f is 2 or higher, substituents in the parenthesis are the same as or different from each other.

6. The heterocyclic compound of claim 1, wherein a deuterium content of the heterocyclic compound of Chemical Formula 1 is 0%, or 10% to 100%.

7. The heterocyclic compound of claim 1, wherein Chemical Formula 1 is represented by any one of the following compounds:

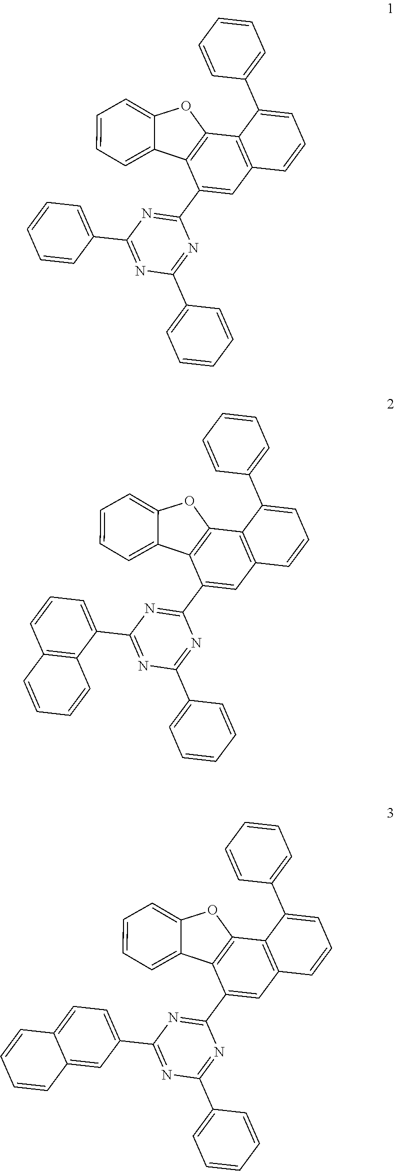 [SMILES: c1ccc(-c2nc(-c3ccccc3)nc(-c3cc4cccc(-c5ccccc5)c4c4oc5ccccc5c34)n2)cc1 ; c1ccc(-c2nc(-c3cccc4ccccc34)nc(-c3cc4cccc(-c5ccccc5)c4c4oc5ccccc5c34)n2)cc1 ; c1ccc(-c2nc(-c3ccc4ccccc4c3)nc(-c3cc4cccc(-c5ccccc5)c4c4oc5ccccc5c34)n2)cc1]

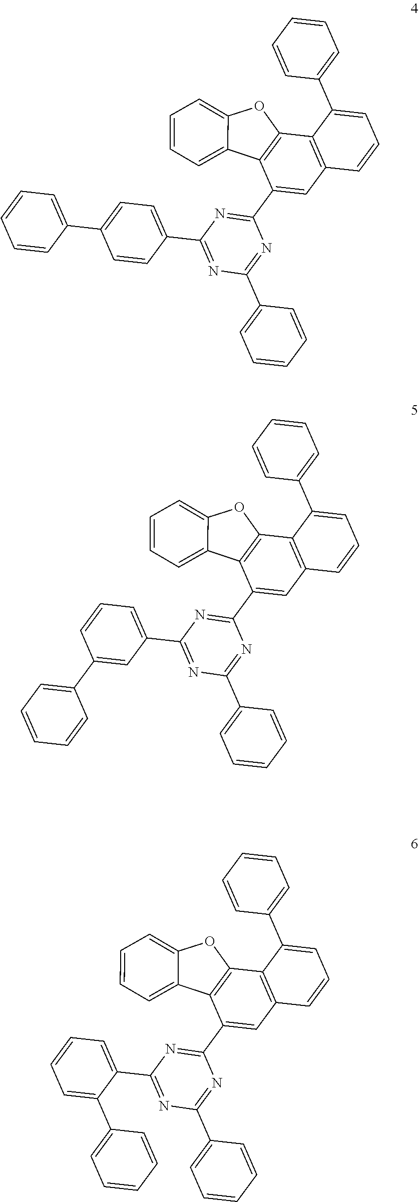 [SMILES: c1ccc(-c2ccc(-c3nc(-c4ccccc4)nc(-c4cc5cccc(-c6ccccc6)c5c5oc6ccccc6c45)n3)cc2)cc1 ; c1ccc(-c2cccc(-c3nc(-c4ccccc4)nc(-c4cc5cccc(-c6ccccc6)c5c5oc6ccccc6c45)n3)c2)cc1 ; c1ccc(-c2nc(-c3ccccc3-c3ccccc3)nc(-c3cc4cccc(-c5ccccc5)c4c4oc5ccccc5c34)n2)cc1]

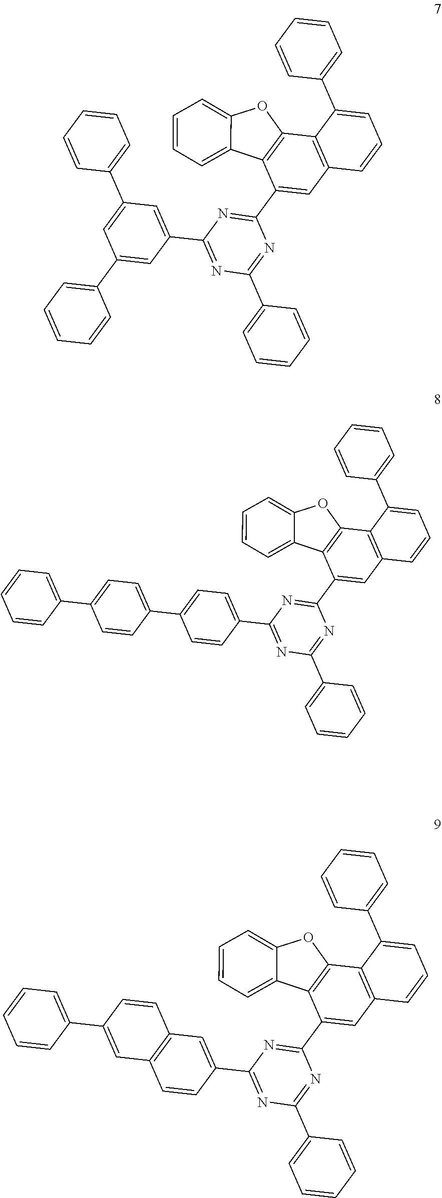 [SMILES: c1ccc(-c2cc(-c3ccccc3)cc(-c3nc(-c4ccccc4)nc(-c4cc5cccc(-c6ccccc6)c5c5oc6ccccc6c45)n3)c2)cc1 ; c1ccc(-c2ccc(-c3ccc(-c4nc(-c5ccccc5)nc(-c5cc6cccc(-c7ccccc7)c6c6oc7ccccc7c56)n4)cc3)cc2)cc1 ; c1ccc(-c2ccc3cc(-c4nc(-c5ccccc5)nc(-c5cc6cccc(-c7ccccc7)c6c6oc7ccccc7c56)n4)ccc3c2)cc1]

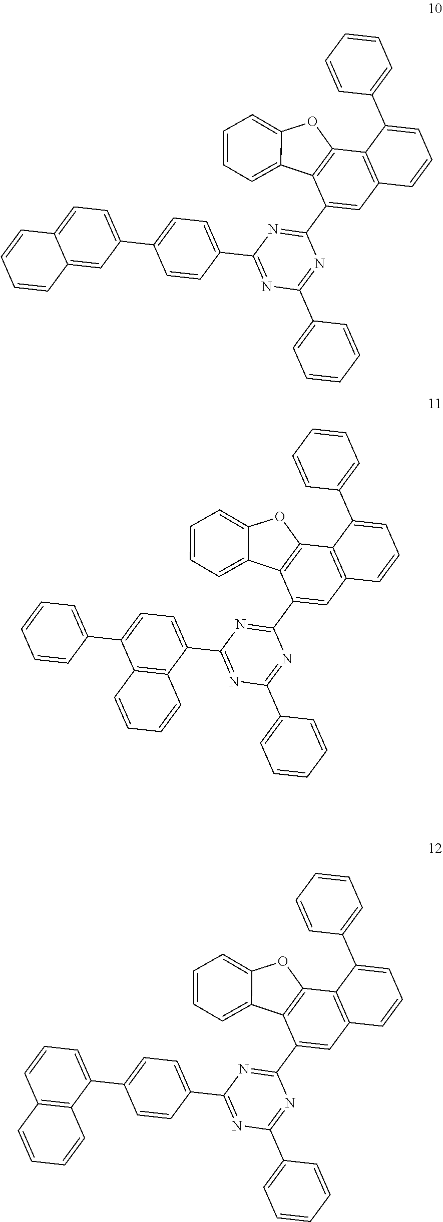 [SMILES: c1ccc(-c2nc(-c3ccc(-c4ccc5ccccc5c4)cc3)nc(-c3cc4cccc(-c5ccccc5)c4c4oc5ccccc5c34)n2)cc1 ; c1ccc(-c2nc(-c3ccc(-c4ccccc4)c4ccccc34)nc(-c3cc4cccc(-c5ccccc5)c4c4oc5ccccc5c34)n2)cc1 ; c1ccc(-c2nc(-c3ccc(-c4cccc5ccccc45)cc3)nc(-c3cc4cccc(-c5ccccc5)c4c4oc5ccccc5c34)n2)cc1]

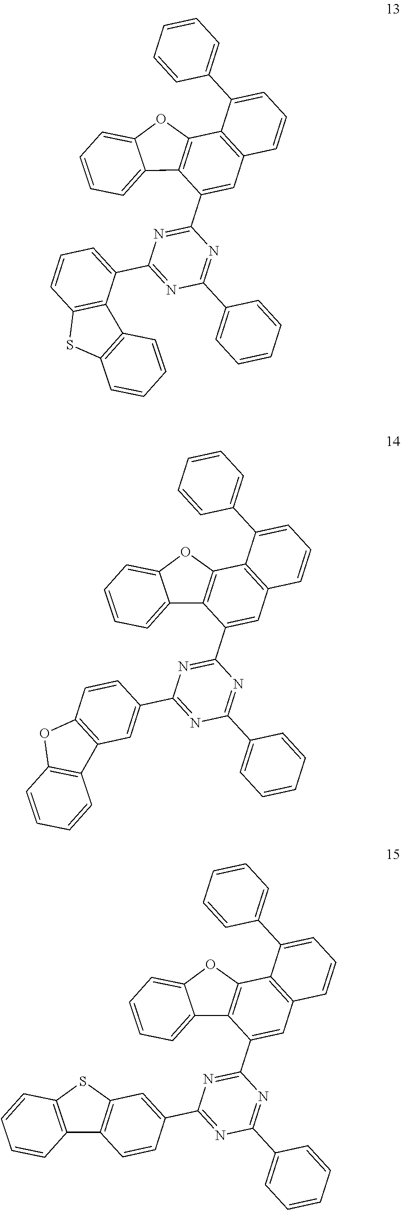 [SMILES: c1ccc(-c2nc(-c3cccc4sc5ccccc5c34)nc(-c3cc4cccc(-c5ccccc5)c4c4oc5ccccc5c34)n2)cc1 ; c1ccc(-c2nc(-c3ccc4oc5ccccc5c4c3)nc(-c3cc4cccc(-c5ccccc5)c4c4oc5ccccc5c34)n2)cc1 ; c1ccc(-c2nc(-c3ccc4c(c3)sc3ccccc34)nc(-c3cc4cccc(-c5ccccc5)c4c4oc5ccccc5c34)n2)cc1]

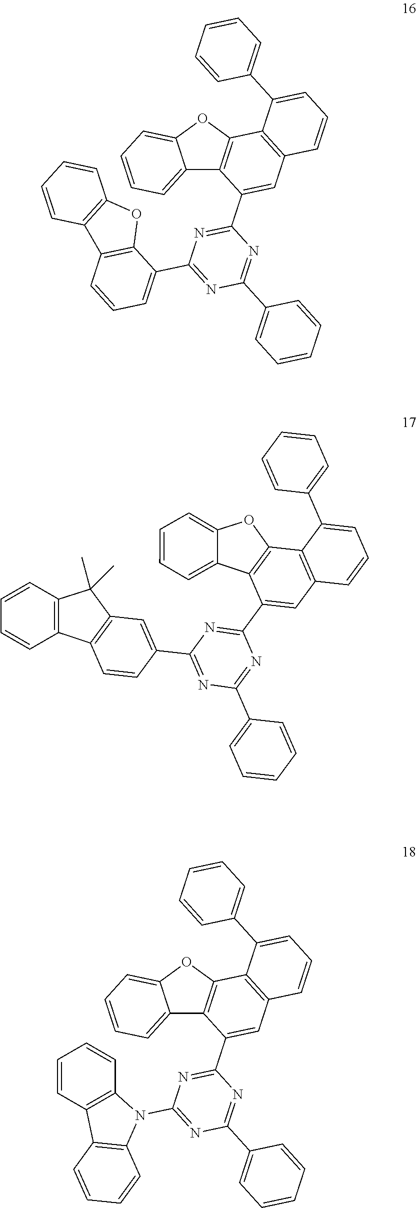 [SMILES: c1ccc(-c2nc(-c3cccc4c3oc3ccccc34)nc(-c3cc4cccc(-c5ccccc5)c4c4oc5ccccc5c34)n2)cc1 ; CC1(C)c2ccccc2-c2ccc(-c3nc(-c4ccccc4)nc(-c4cc5cccc(-c6ccccc6)c5c5oc6ccccc6c45)n3)cc21 ; c1ccc(-c2nc(-c3cc4cccc(-c5ccccc5)c4c4oc5ccccc5c34)nc(-n3c4ccccc4c4ccccc43)n2)cc1]

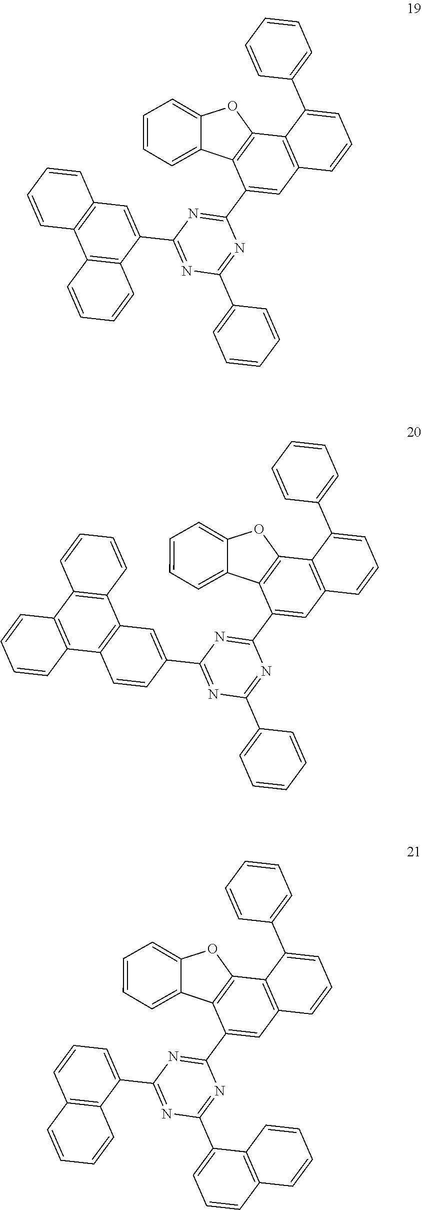 [SMILES: c1ccc(-c2nc(-c3cc4ccccc4c4ccccc34)nc(-c3cc4cccc(-c5ccccc5)c4c4oc5ccccc5c34)n2)cc1 ; c1ccc(-c2nc(-c3ccc4c5ccccc5c5ccccc5c4c3)nc(-c3cc4cccc(-c5ccccc5)c4c4oc5ccccc5c34)n2)cc1 ; c1ccc(-c2cccc3cc(-c4nc(-c5cccc6ccccc56)nc(-c5cccc6ccccc56)n4)c4c5ccccc5oc4c23)cc1]

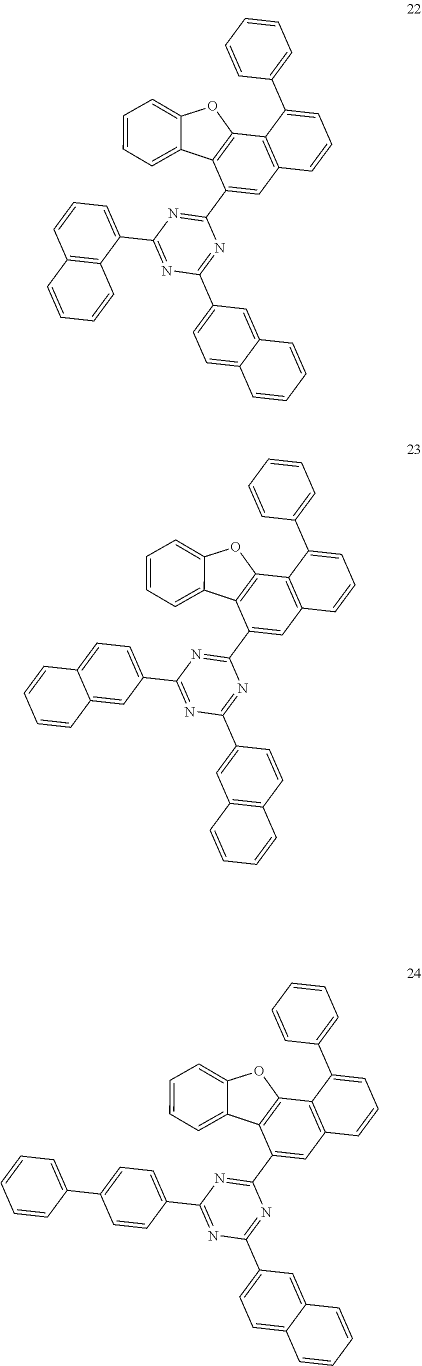 [SMILES: c1ccc(-c2cccc3cc(-c4nc(-c5ccc6ccccc6c5)nc(-c5cccc6ccccc56)n4)c4c5ccccc5oc4c23)cc1 ; c1ccc(-c2cccc3cc(-c4nc(-c5ccc6ccccc6c5)nc(-c5ccc6ccccc6c5)n4)c4c5ccccc5oc4c23)cc1 ; c1ccc(-c2ccc(-c3nc(-c4ccc5ccccc5c4)nc(-c4cc5cccc(-c6ccccc6)c5c5oc6ccccc6c45)n3)cc2)cc1]

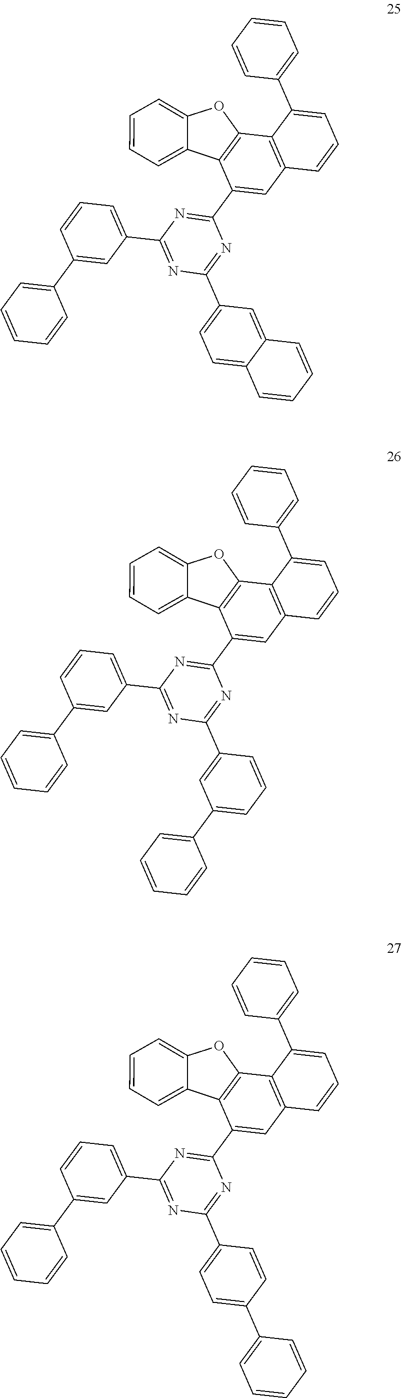 [SMILES: c1ccc(-c2cccc(-c3nc(-c4ccc5ccccc5c4)nc(-c4cc5cccc(-c6ccccc6)c5c5oc6ccccc6c45)n3)c2)cc1 ; c1ccc(-c2cccc(-c3nc(-c4cccc(-c5ccccc5)c4)nc(-c4cc5cccc(-c6ccccc6)c5c5oc6ccccc6c45)n3)c2)cc1 ; c1ccc(-c2ccc(-c3nc(-c4cccc(-c5ccccc5)c4)nc(-c4cc5cccc(-c6ccccc6)c5c5oc6ccccc6c45)n3)cc2)cc1]

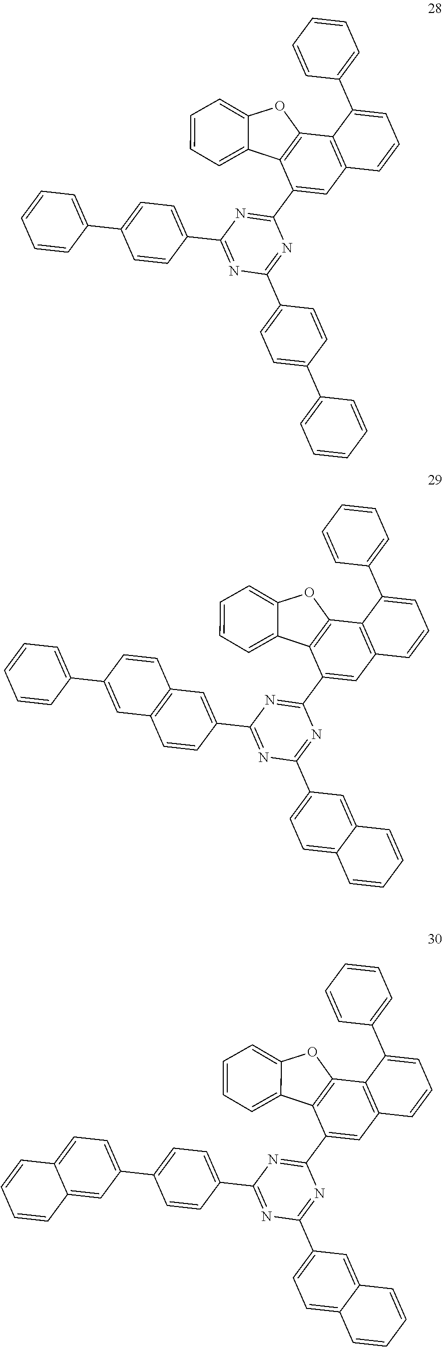 [SMILES: c1ccc(-c2ccc(-c3nc(-c4ccc(-c5ccccc5)cc4)nc(-c4cc5cccc(-c6ccccc6)c5c5oc6ccccc6c45)n3)cc2)cc1 ; c1ccc(-c2ccc3cc(-c4nc(-c5ccc6ccccc6c5)nc(-c5cc6cccc(-c7ccccc7)c6c6oc7ccccc7c56)n4)ccc3c2)cc1 ; c1ccc(-c2cccc3cc(-c4nc(-c5ccc(-c6ccc7ccccc7c6)cc5)nc(-c5ccc6ccccc6c5)n4)c4c5ccccc5oc4c23)cc1]

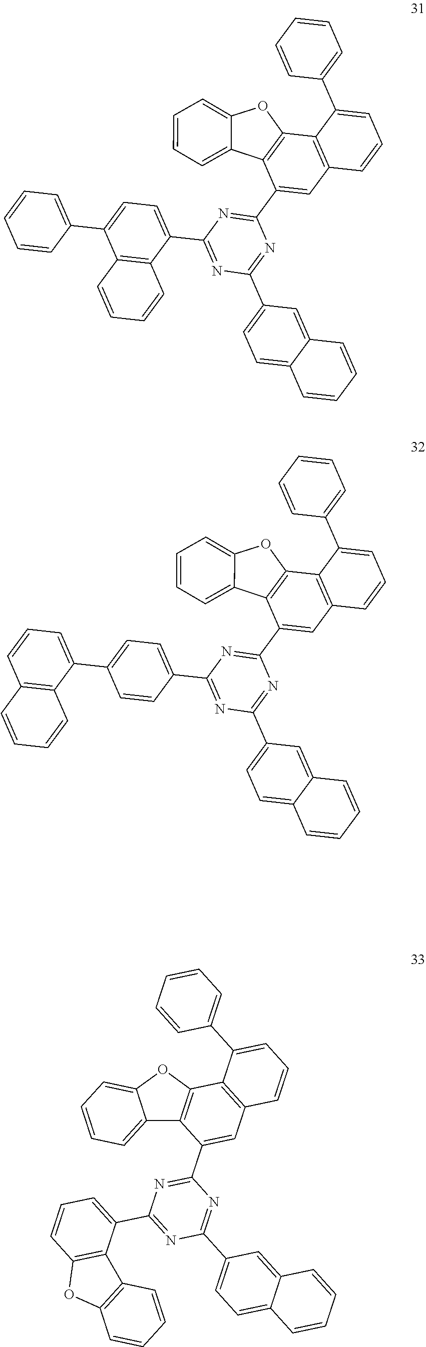 [SMILES: c1ccc(-c2ccc(-c3nc(-c4ccc5ccccc5c4)nc(-c4cc5cccc(-c6ccccc6)c5c5oc6ccccc6c45)n3)c3ccccc23)cc1 ; c1ccc(-c2cccc3cc(-c4nc(-c5ccc(-c6cccc7ccccc67)cc5)nc(-c5ccc6ccccc6c5)n4)c4c5ccccc5oc4c23)cc1 ; c1ccc(-c2cccc3cc(-c4nc(-c5ccc6ccccc6c5)nc(-c5cccc6oc7ccccc7c56)n4)c4c5ccccc5oc4c23)cc1]

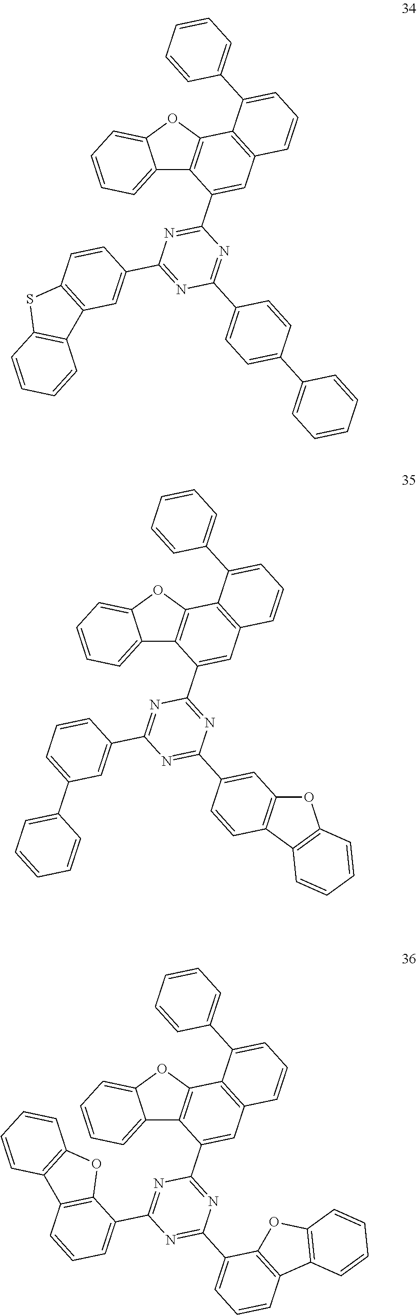 [SMILES: c1ccc(-c2ccc(-c3nc(-c4ccc5sc6ccccc6c5c4)nc(-c4cc5cccc(-c6ccccc6)c5c5oc6ccccc6c45)n3)cc2)cc1 ; c1ccc(-c2cccc(-c3nc(-c4ccc5c(c4)oc4ccccc45)nc(-c4cc5cccc(-c6ccccc6)c5c5oc6ccccc6c45)n3)c2)cc1 ; c1ccc(-c2cccc3cc(-c4nc(-c5cccc6c5oc5ccccc56)nc(-c5cccc6c5oc5ccccc56)n4)c4c5ccccc5oc4c23)cc1]

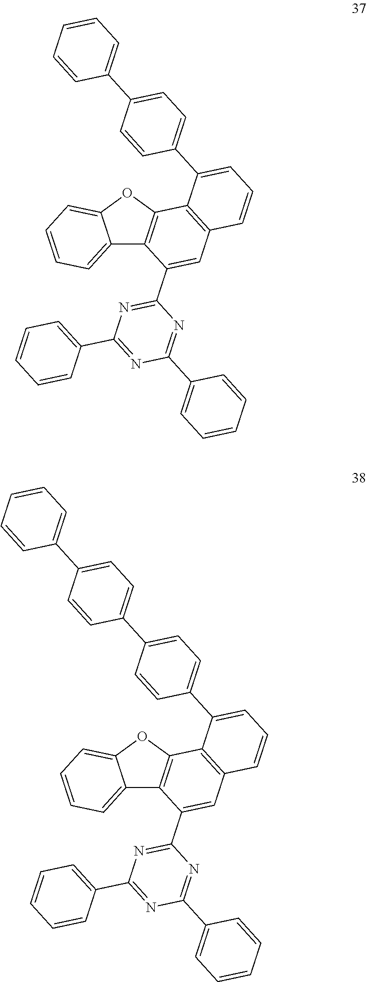 [SMILES: c1ccc(-c2ccc(-c3cccc4cc(-c5nc(-c6ccccc6)nc(-c6ccccc6)n5)c5c6ccccc6oc5c34)cc2)cc1 ; c1ccc(-c2ccc(-c3ccc(-c4cccc5cc(-c6nc(-c7ccccc7)nc(-c7ccccc7)n6)c6c7ccccc7oc6c45)cc3)cc2)cc1]

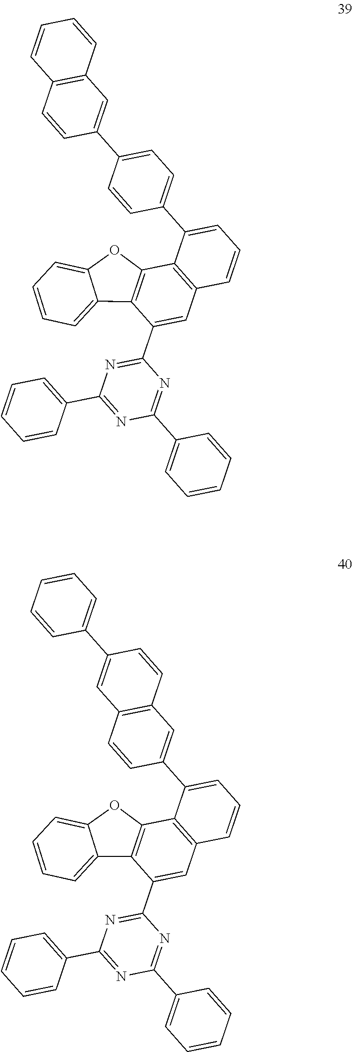 [SMILES: c1ccc(-c2nc(-c3ccccc3)nc(-c3cc4cccc(-c5ccc(-c6ccc7ccccc7c6)cc5)c4c4oc5ccccc5c34)n2)cc1 ; c1ccc(-c2ccc3cc(-c4cccc5cc(-c6nc(-c7ccccc7)nc(-c7ccccc7)n6)c6c7ccccc7oc6c45)ccc3c2)cc1]

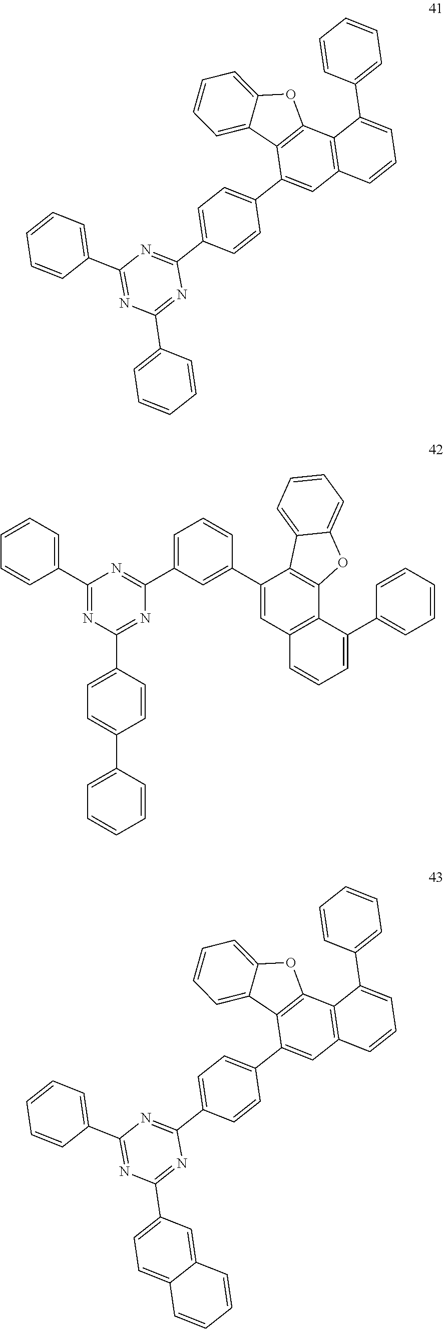 [SMILES: c1ccc(-c2nc(-c3ccccc3)nc(-c3ccc(-c4cc5cccc(-c6ccccc6)c5c5oc6ccccc6c45)cc3)n2)cc1 ; c1ccc(-c2ccc(-c3nc(-c4ccccc4)nc(-c4cccc(-c5cc6cccc(-c7ccccc7)c6c6oc7ccccc7c56)c4)n3)cc2)cc1 ; c1ccc(-c2nc(-c3ccc(-c4cc5cccc(-c6ccccc6)c5c5oc6ccccc6c45)cc3)nc(-c3ccc4ccccc4c3)n2)cc1]

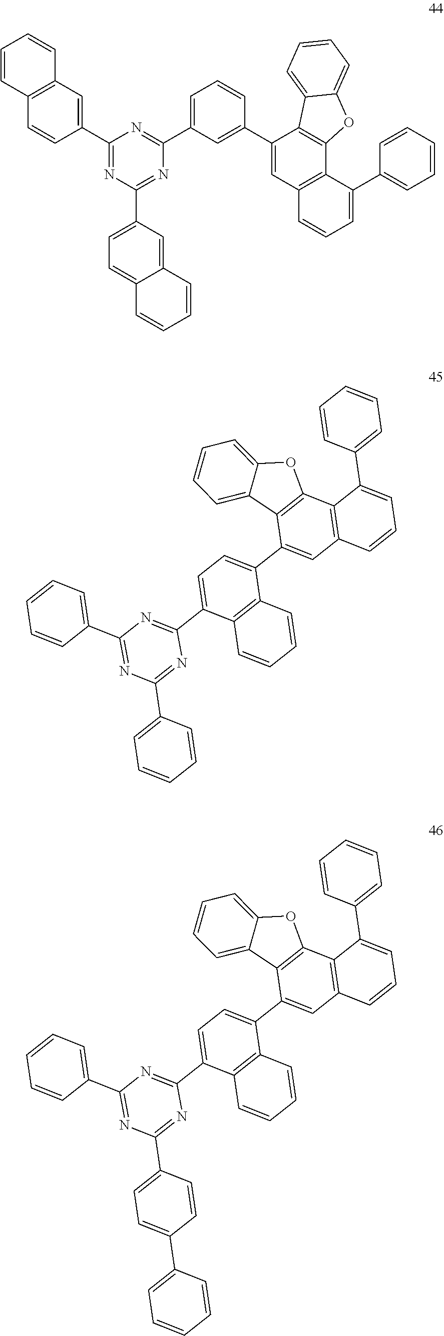 [SMILES: c1ccc(-c2cccc3cc(-c4cccc(-c5nc(-c6ccc7ccccc7c6)nc(-c6ccc7ccccc7c6)n5)c4)c4c5ccccc5oc4c23)cc1 ; c1ccc(-c2nc(-c3ccccc3)nc(-c3ccc(-c4cc5cccc(-c6ccccc6)c5c5oc6ccccc6c45)c4ccccc34)n2)cc1 ; c1ccc(-c2ccc(-c3nc(-c4ccccc4)nc(-c4ccc(-c5cc6cccc(-c7ccccc7)c6c6oc7ccccc7c56)c5ccccc45)n3)cc2)cc1]

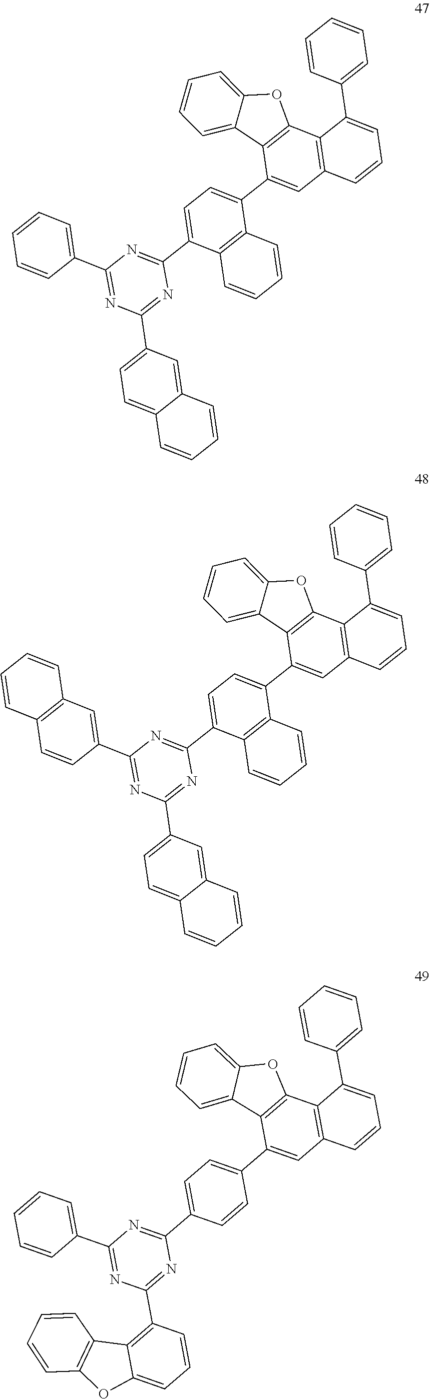 [SMILES: c1ccc(-c2nc(-c3ccc4ccccc4c3)nc(-c3ccc(-c4cc5cccc(-c6ccccc6)c5c5oc6ccccc6c45)c4ccccc34)n2)cc1 ; c1ccc(-c2cccc3cc(-c4ccc(-c5nc(-c6ccc7ccccc7c6)nc(-c6ccc7ccccc7c6)n5)c5ccccc45)c4c5ccccc5oc4c23)cc1 ; c1ccc(-c2nc(-c3ccc(-c4cc5cccc(-c6ccccc6)c5c5oc6ccccc6c45)cc3)nc(-c3cccc4oc5ccccc5c34)n2)cc1]

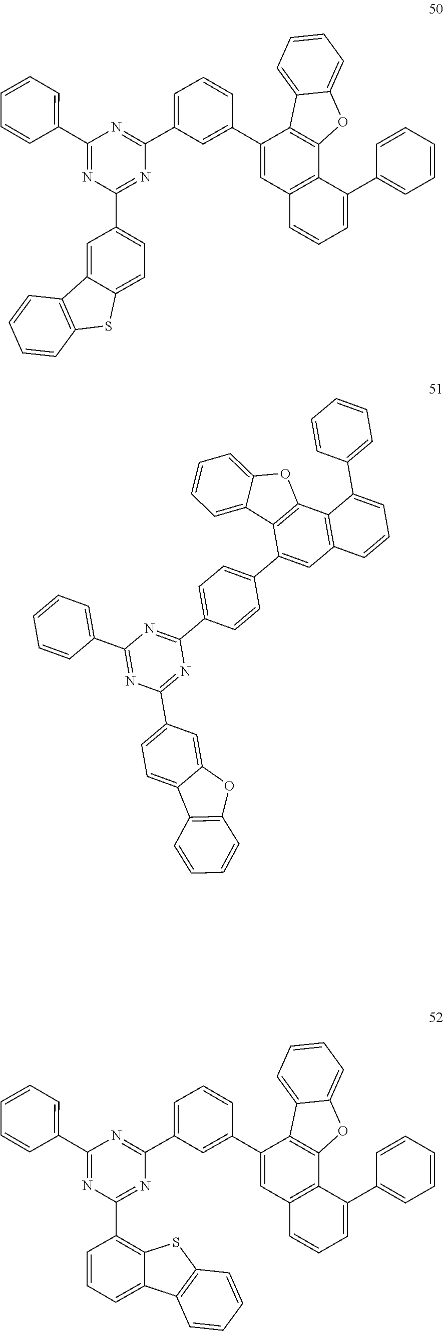 [SMILES: c1ccc(-c2nc(-c3cccc(-c4cc5cccc(-c6ccccc6)c5c5oc6ccccc6c45)c3)nc(-c3ccc4sc5ccccc5c4c3)n2)cc1 ; c1ccc(-c2nc(-c3ccc(-c4cc5cccc(-c6ccccc6)c5c5oc6ccccc6c45)cc3)nc(-c3ccc4c(c3)oc3ccccc34)n2)cc1 ; c1ccc(-c2nc(-c3cccc(-c4cc5cccc(-c6ccccc6)c5c5oc6ccccc6c45)c3)nc(-c3cccc4c3sc3ccccc34)n2)cc1]

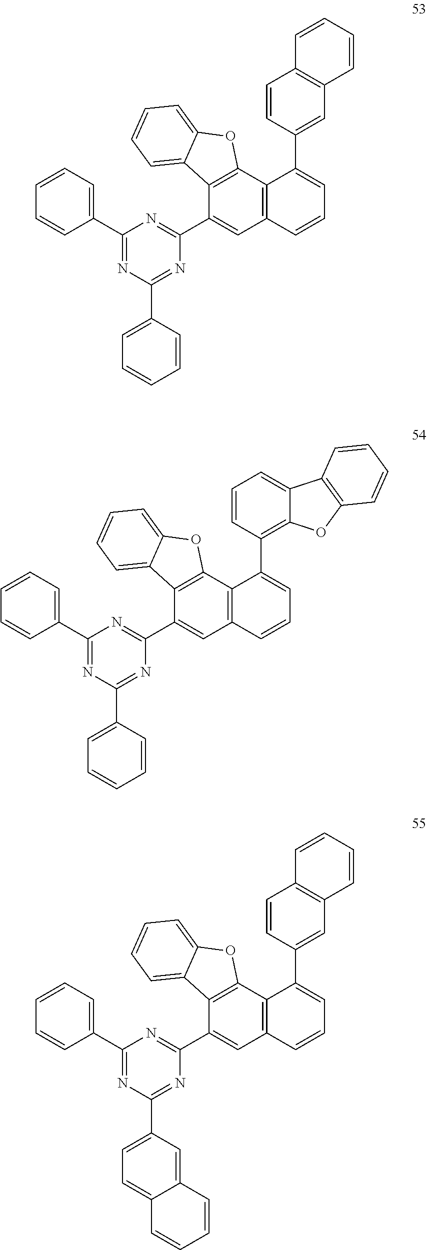 [SMILES: c1ccc(-c2nc(-c3ccccc3)nc(-c3cc4cccc(-c5ccc6ccccc6c5)c4c4oc5ccccc5c34)n2)cc1 ; c1ccc(-c2nc(-c3ccccc3)nc(-c3cc4cccc(-c5cccc6c5oc5ccccc56)c4c4oc5ccccc5c34)n2)cc1 ; c1ccc(-c2nc(-c3ccc4ccccc4c3)nc(-c3cc4cccc(-c5ccc6ccccc6c5)c4c4oc5ccccc5c34)n2)cc1]

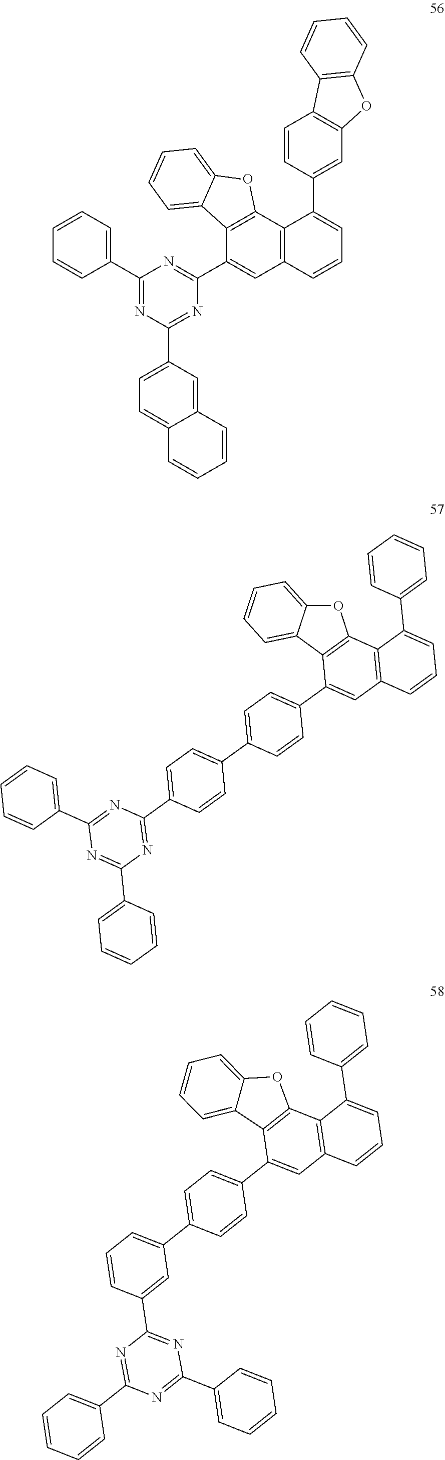 [SMILES: c1ccc(-c2nc(-c3ccc4ccccc4c3)nc(-c3cc4cccc(-c5ccc6c(c5)oc5ccccc56)c4c4oc5ccccc5c34)n2)cc1 ; c1ccc(-c2nc(-c3ccccc3)nc(-c3ccc(-c4ccc(-c5cc6cccc(-c7ccccc7)c6c6oc7ccccc7c56)cc4)cc3)n2)cc1 ; c1ccc(-c2nc(-c3ccccc3)nc(-c3cccc(-c4ccc(-c5cc6cccc(-c7ccccc7)c6c6oc7ccccc7c56)cc4)c3)n2)cc1]

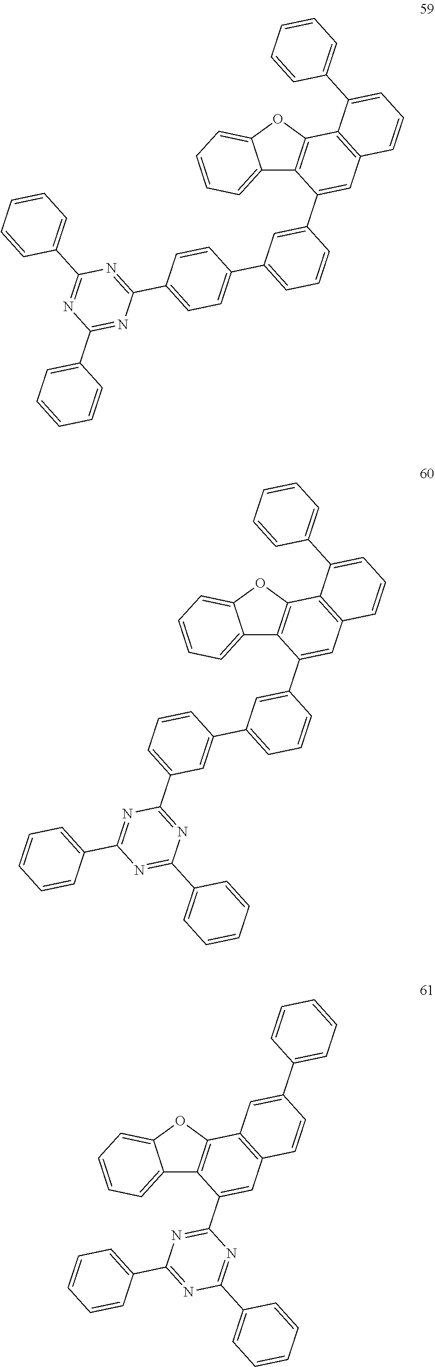 [SMILES: c1ccc(-c2nc(-c3ccccc3)nc(-c3ccc(-c4cccc(-c5cc6cccc(-c7ccccc7)c6c6oc7ccccc7c56)c4)cc3)n2)cc1 ; c1ccc(-c2nc(-c3ccccc3)nc(-c3cccc(-c4cccc(-c5cc6cccc(-c7ccccc7)c6c6oc7ccccc7c56)c4)c3)n2)cc1 ; c1ccc(-c2ccc3cc(-c4nc(-c5ccccc5)nc(-c5ccccc5)n4)c4c5ccccc5oc4c3c2)cc1]

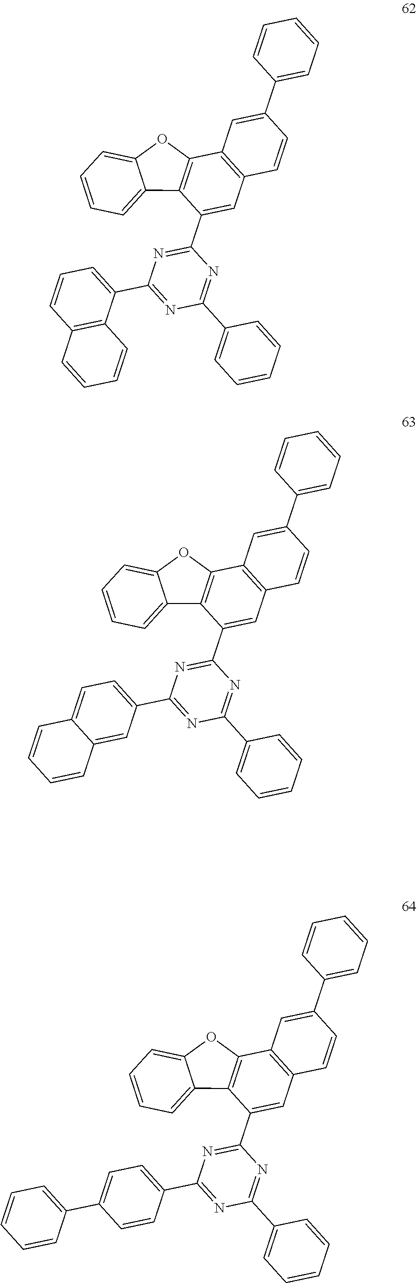 [SMILES: c1ccc(-c2ccc3cc(-c4nc(-c5ccccc5)nc(-c5cccc6ccccc56)n4)c4c5ccccc5oc4c3c2)cc1 ; c1ccc(-c2ccc3cc(-c4nc(-c5ccccc5)nc(-c5ccc6ccccc6c5)n4)c4c5ccccc5oc4c3c2)cc1 ; c1ccc(-c2ccc(-c3nc(-c4ccccc4)nc(-c4cc5ccc(-c6ccccc6)cc5c5oc6ccccc6c45)n3)cc2)cc1]

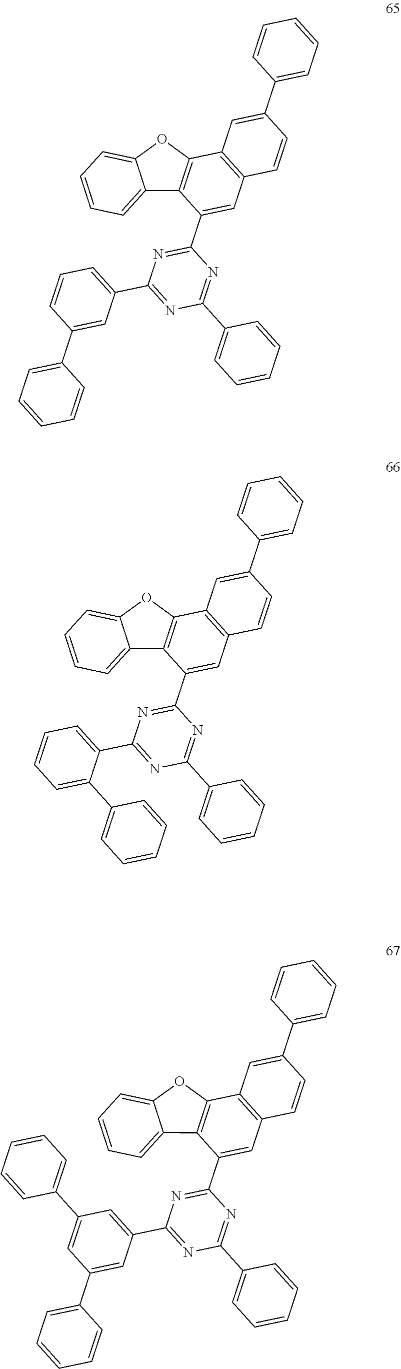 [SMILES: c1ccc(-c2cccc(-c3nc(-c4ccccc4)nc(-c4cc5ccc(-c6ccccc6)cc5c5oc6ccccc6c45)n3)c2)cc1 ; c1ccc(-c2ccc3cc(-c4nc(-c5ccccc5)nc(-c5ccccc5-c5ccccc5)n4)c4c5ccccc5oc4c3c2)cc1 ; c1ccc(-c2cc(-c3ccccc3)cc(-c3nc(-c4ccccc4)nc(-c4cc5ccc(-c6ccccc6)cc5c5oc6ccccc6c45)n3)c2)cc1]

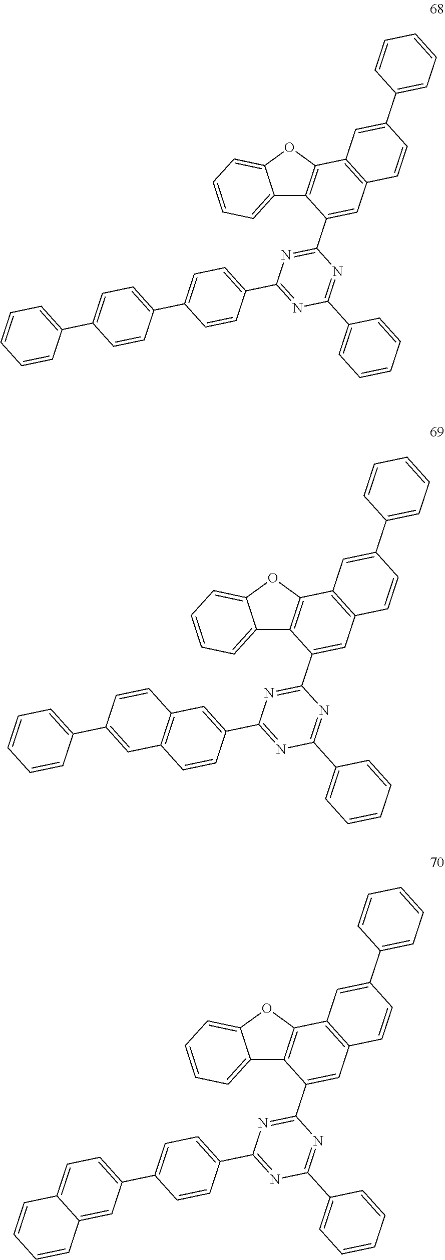 [SMILES: c1ccc(-c2ccc(-c3ccc(-c4nc(-c5ccccc5)nc(-c5cc6ccc(-c7ccccc7)cc6c6oc7ccccc7c56)n4)cc3)cc2)cc1 ; c1ccc(-c2ccc3cc(-c4nc(-c5ccccc5)nc(-c5cc6ccc(-c7ccccc7)cc6c6oc7ccccc7c56)n4)ccc3c2)cc1 ; c1ccc(-c2ccc3cc(-c4nc(-c5ccccc5)nc(-c5ccc(-c6ccc7ccccc7c6)cc5)n4)c4c5ccccc5oc4c3c2)cc1]

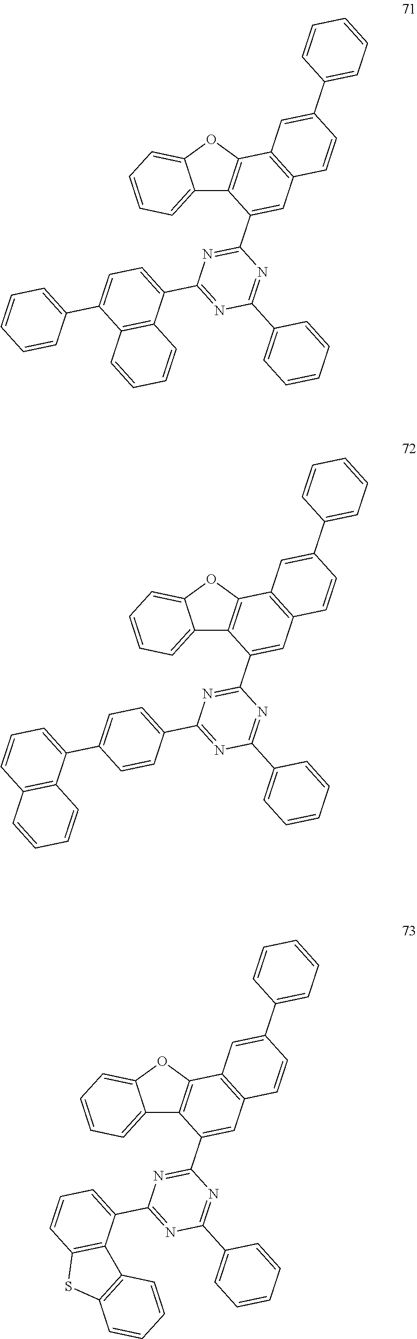 [SMILES: c1ccc(-c2ccc3cc(-c4nc(-c5ccccc5)nc(-c5ccc(-c6ccccc6)c6ccccc56)n4)c4c5ccccc5oc4c3c2)cc1 ; c1ccc(-c2ccc3cc(-c4nc(-c5ccccc5)nc(-c5ccc(-c6cccc7ccccc67)cc5)n4)c4c5ccccc5oc4c3c2)cc1 ; c1ccc(-c2ccc3cc(-c4nc(-c5ccccc5)nc(-c5cccc6sc7ccccc7c56)n4)c4c5ccccc5oc4c3c2)cc1]

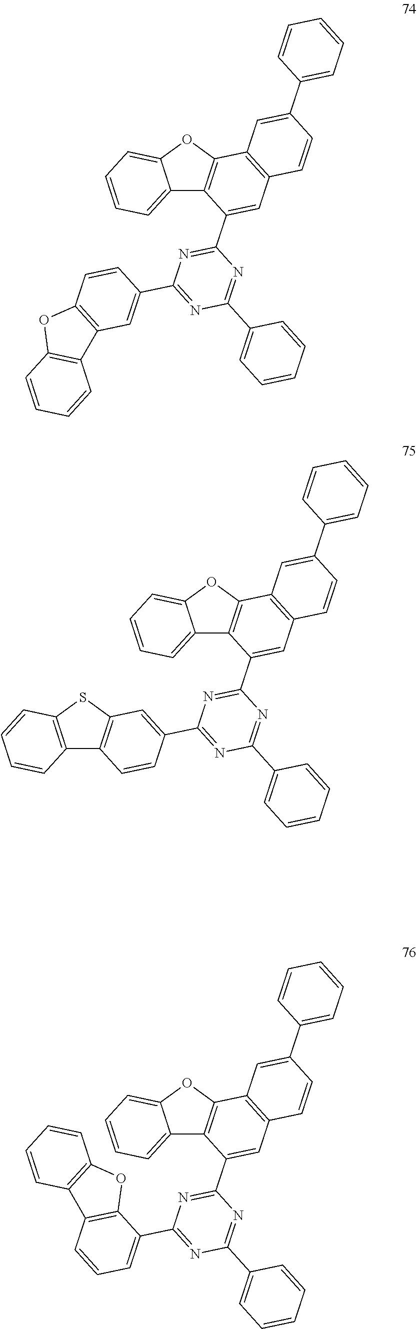 [SMILES: c1ccc(-c2ccc3cc(-c4nc(-c5ccccc5)nc(-c5ccc6oc7ccccc7c6c5)n4)c4c5ccccc5oc4c3c2)cc1 ; c1ccc(-c2ccc3cc(-c4nc(-c5ccccc5)nc(-c5ccc6c(c5)sc5ccccc56)n4)c4c5ccccc5oc4c3c2)cc1 ; c1ccc(-c2ccc3cc(-c4nc(-c5ccccc5)nc(-c5cccc6c5oc5ccccc56)n4)c4c5ccccc5oc4c3c2)cc1]

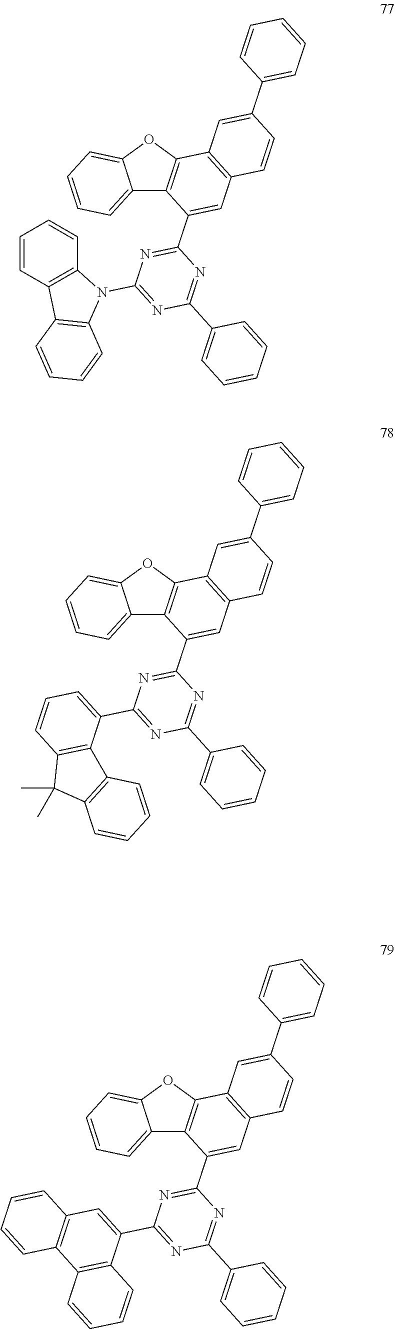 [SMILES: c1ccc(-c2ccc3cc(-c4nc(-c5ccccc5)nc(-n5c6ccccc6c6ccccc65)n4)c4c5ccccc5oc4c3c2)cc1 ; CC1(C)c2ccccc2-c2c(-c3nc(-c4ccccc4)nc(-c4cc5ccc(-c6ccccc6)cc5c5oc6ccccc6c45)n3)cccc21 ; c1ccc(-c2ccc3cc(-c4nc(-c5ccccc5)nc(-c5cc6ccccc6c6ccccc56)n4)c4c5ccccc5oc4c3c2)cc1]

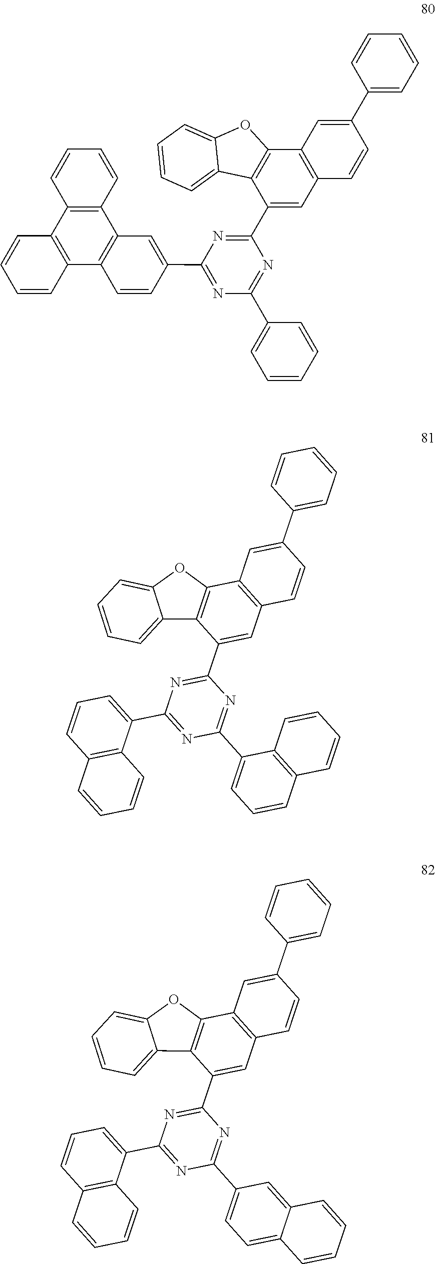 [SMILES: c1ccc(-c2ccc3cc(-c4nc(-c5ccccc5)nc(-c5ccc6c7ccccc7c7ccccc7c6c5)n4)c4c5ccccc5oc4c3c2)cc1 ; c1ccc(-c2ccc3cc(-c4nc(-c5cccc6ccccc56)nc(-c5cccc6ccccc56)n4)c4c5ccccc5oc4c3c2)cc1 ; c1ccc(-c2ccc3cc(-c4nc(-c5ccc6ccccc6c5)nc(-c5cccc6ccccc56)n4)c4c5ccccc5oc4c3c2)cc1]

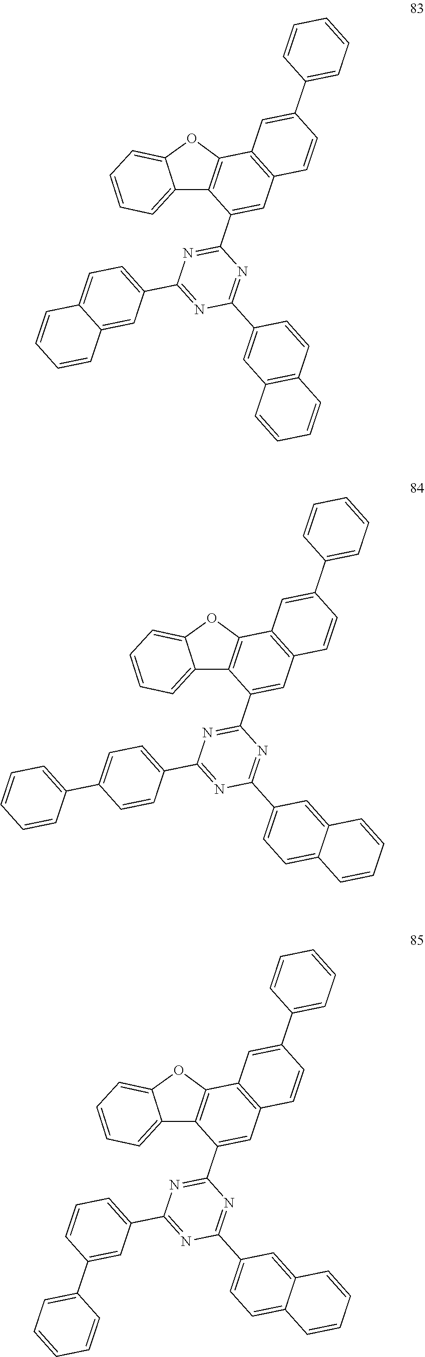 [SMILES: c1ccc(-c2ccc3cc(-c4nc(-c5ccc6ccccc6c5)nc(-c5ccc6ccccc6c5)n4)c4c5ccccc5oc4c3c2)cc1 ; c1ccc(-c2ccc(-c3nc(-c4ccc5ccccc5c4)nc(-c4cc5ccc(-c6ccccc6)cc5c5oc6ccccc6c45)n3)cc2)cc1 ; c1ccc(-c2cccc(-c3nc(-c4ccc5ccccc5c4)nc(-c4cc5ccc(-c6ccccc6)cc5c5oc6ccccc6c45)n3)c2)cc1]

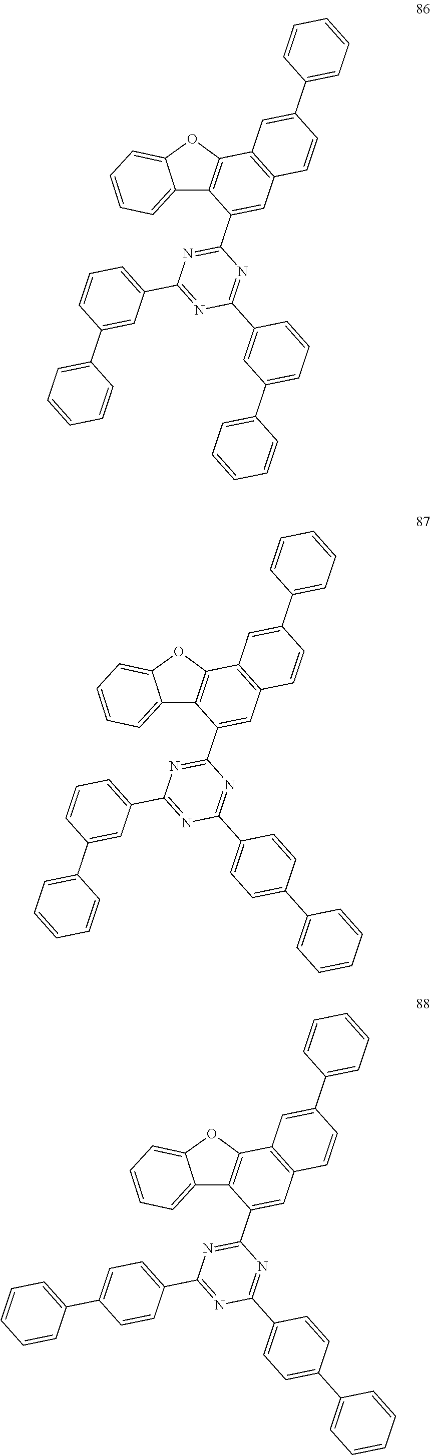 [SMILES: c1ccc(-c2cccc(-c3nc(-c4cccc(-c5ccccc5)c4)nc(-c4cc5ccc(-c6ccccc6)cc5c5oc6ccccc6c45)n3)c2)cc1 ; c1ccc(-c2ccc(-c3nc(-c4cccc(-c5ccccc5)c4)nc(-c4cc5ccc(-c6ccccc6)cc5c5oc6ccccc6c45)n3)cc2)cc1 ; c1ccc(-c2ccc(-c3nc(-c4ccc(-c5ccccc5)cc4)nc(-c4cc5ccc(-c6ccccc6)cc5c5oc6ccccc6c45)n3)cc2)cc1]

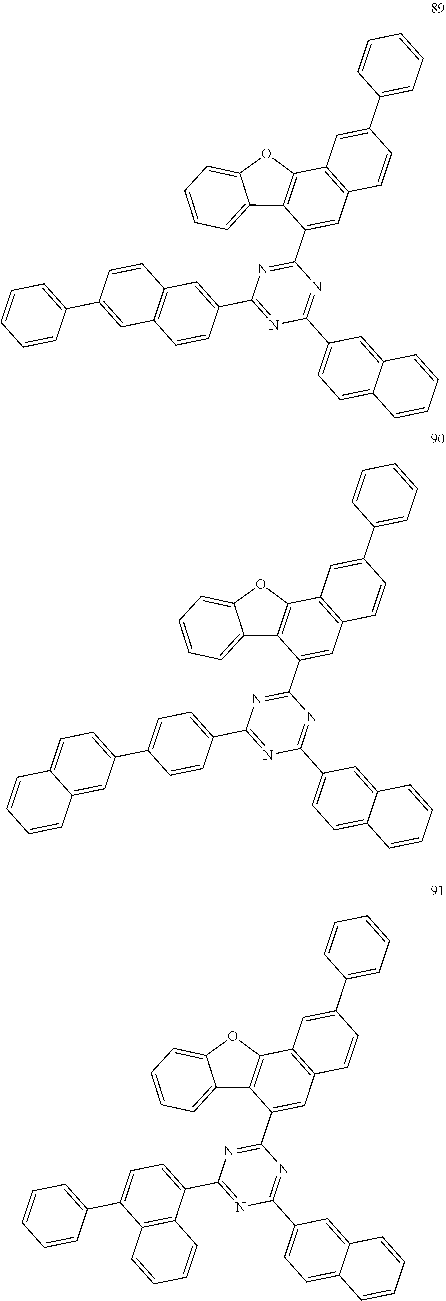

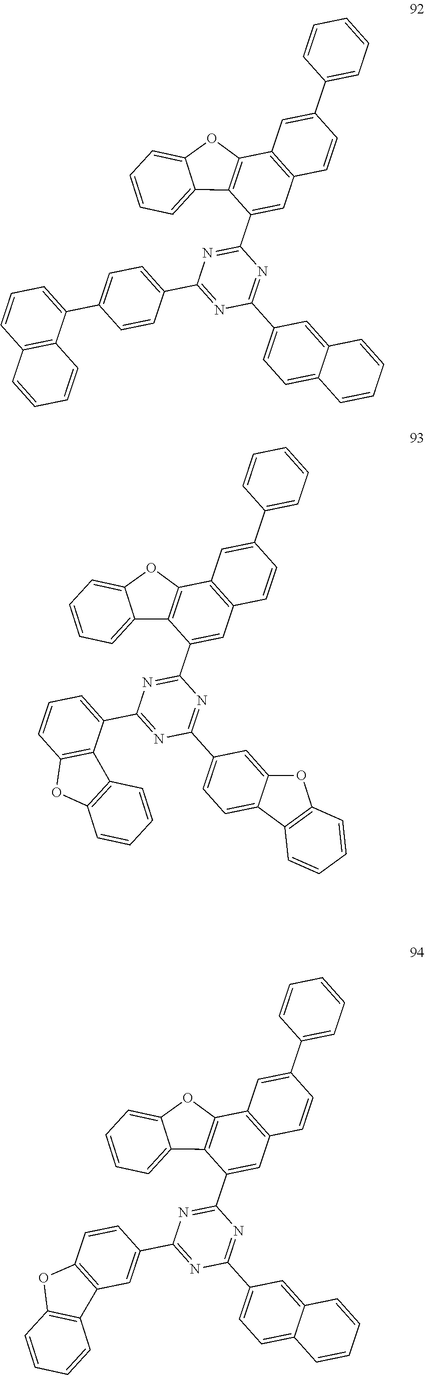 [SMILES: c1ccc(-c2ccc3cc(-c4nc(-c5ccc(-c6cccc7ccccc67)cc5)nc(-c5ccc6ccccc6c5)n4)c4c5ccccc5oc4c3c2)cc1 ; c1ccc(-c2ccc3cc(-c4nc(-c5ccc6c(c5)oc5ccccc56)nc(-c5cccc6oc7ccccc7c56)n4)c4c5ccccc5oc4c3c2)cc1 ; c1ccc(-c2ccc3cc(-c4nc(-c5ccc6ccccc6c5)nc(-c5ccc6oc7ccccc7c6c5)n4)c4c5ccccc5oc4c3c2)cc1]

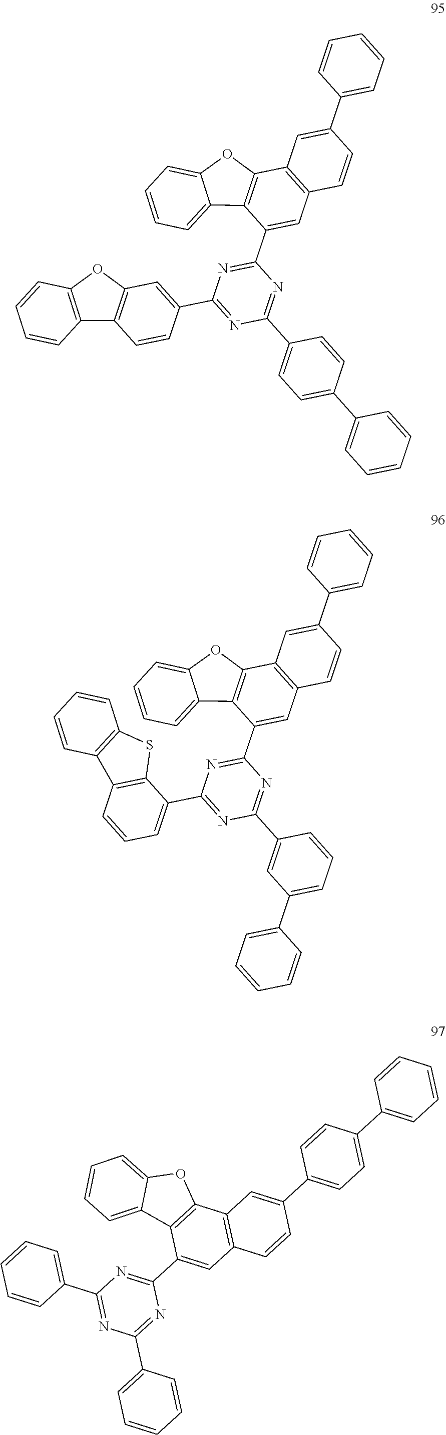 [SMILES: c1ccc(-c2ccc(-c3nc(-c4ccc5c(c4)oc4ccccc45)nc(-c4cc5ccc(-c6ccccc6)cc5c5oc6ccccc6c45)n3)cc2)cc1 ; c1ccc(-c2cccc(-c3nc(-c4cccc5c4sc4ccccc45)nc(-c4cc5ccc(-c6ccccc6)cc5c5oc6ccccc6c45)n3)c2)cc1 ; c1ccc(-c2ccc(-c3ccc4cc(-c5nc(-c6ccccc6)nc(-c6ccccc6)n5)c5c6ccccc6oc5c4c3)cc2)cc1]

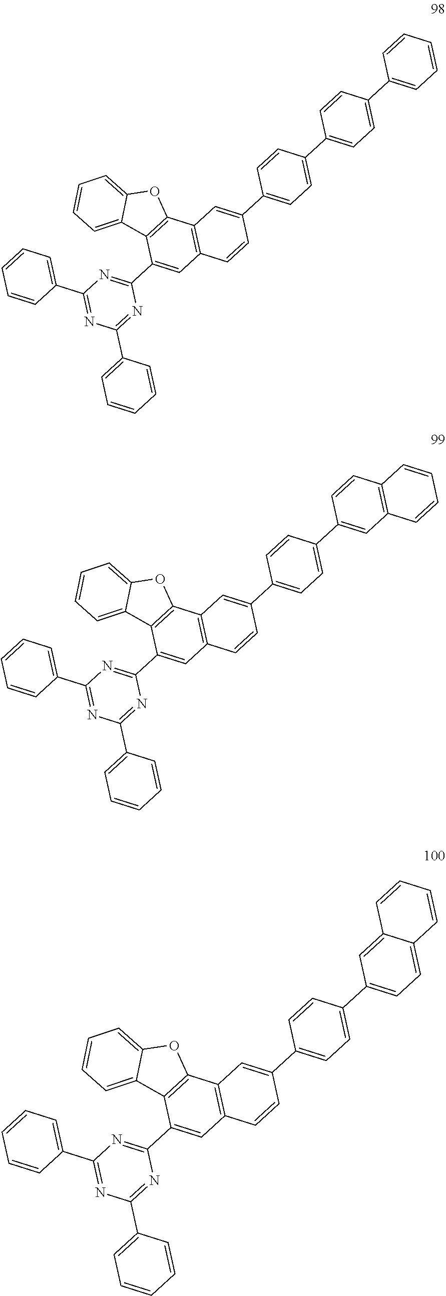 [SMILES: c1ccc(-c2ccc(-c3ccc(-c4ccc5cc(-c6nc(-c7ccccc7)nc(-c7ccccc7)n6)c6c7ccccc7oc6c5c4)cc3)cc2)cc1 ; c1ccc(-c2nc(-c3ccccc3)nc(-c3cc4ccc(-c5ccc(-c6ccc7ccccc7c6)cc5)cc4c4oc5ccccc5c34)n2)cc1 ; c1ccc(-c2nc(-c3ccccc3)nc(-c3cc4ccc(-c5ccc(-c6ccc7ccccc7c6)cc5)cc4c4oc5ccccc5c34)n2)cc1]

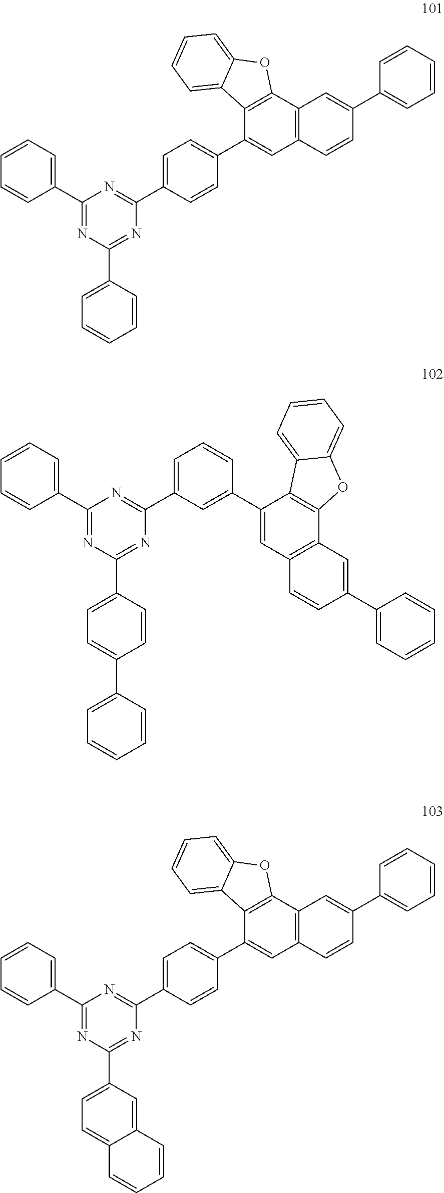 [SMILES: c1ccc(-c2ccc3cc(-c4ccc(-c5nc(-c6ccccc6)nc(-c6ccccc6)n5)cc4)c4c5ccccc5oc4c3c2)cc1 ; c1ccc(-c2ccc(-c3nc(-c4ccccc4)nc(-c4cccc(-c5cc6ccc(-c7ccccc7)cc6c6oc7ccccc7c56)c4)n3)cc2)cc1 ; c1ccc(-c2ccc3cc(-c4ccc(-c5nc(-c6ccccc6)nc(-c6ccc7ccccc7c6)n5)cc4)c4c5ccccc5oc4c3c2)cc1]

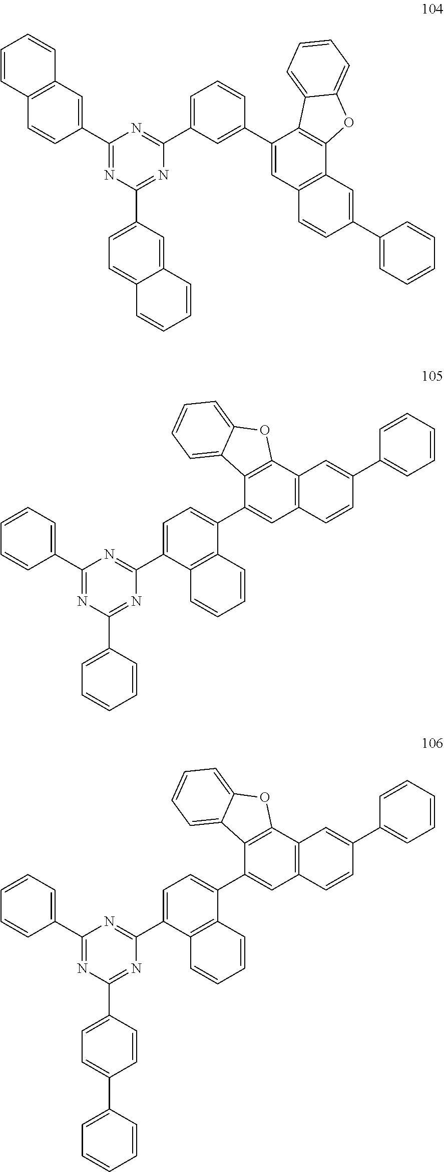 [SMILES: c1ccc(-c2ccc3cc(-c4cccc(-c5nc(-c6ccc7ccccc7c6)nc(-c6ccc7ccccc7c6)n5)c4)c4c5ccccc5oc4c3c2)cc1 ; c1ccc(-c2ccc3cc(-c4ccc(-c5nc(-c6ccccc6)nc(-c6ccccc6)n5)c5ccccc45)c4c5ccccc5oc4c3c2)cc1 ; c1ccc(-c2ccc(-c3nc(-c4ccccc4)nc(-c4ccc(-c5cc6ccc(-c7ccccc7)cc6c6oc7ccccc7c56)c5ccccc45)n3)cc2)cc1]

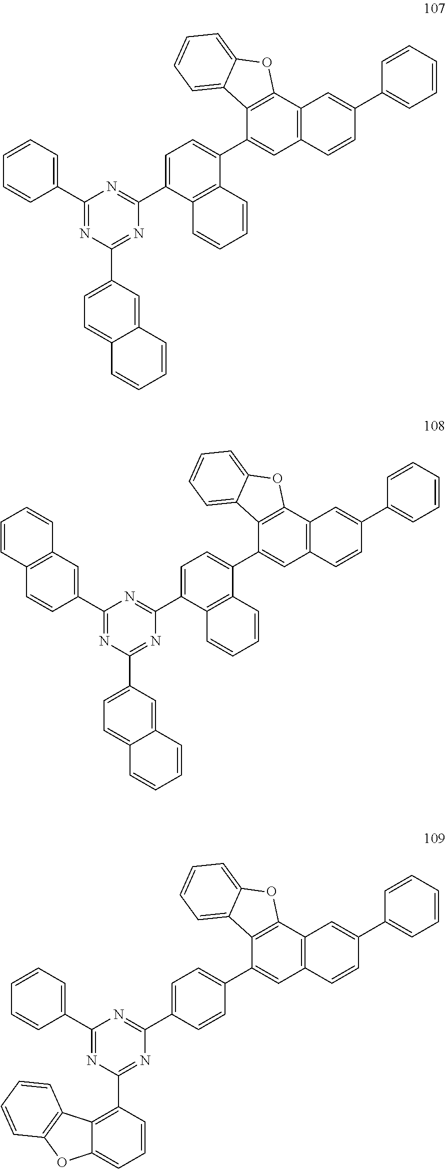 [SMILES: c1ccc(-c2ccc3cc(-c4ccc(-c5nc(-c6ccccc6)nc(-c6ccc7ccccc7c6)n5)c5ccccc45)c4c5ccccc5oc4c3c2)cc1 ; c1ccc(-c2ccc3cc(-c4ccc(-c5nc(-c6ccc7ccccc7c6)nc(-c6ccc7ccccc7c6)n5)c5ccccc45)c4c5ccccc5oc4c3c2)cc1 ; c1ccc(-c2ccc3cc(-c4ccc(-c5nc(-c6ccccc6)nc(-c6cccc7oc8ccccc8c67)n5)cc4)c4c5ccccc5oc4c3c2)cc1]

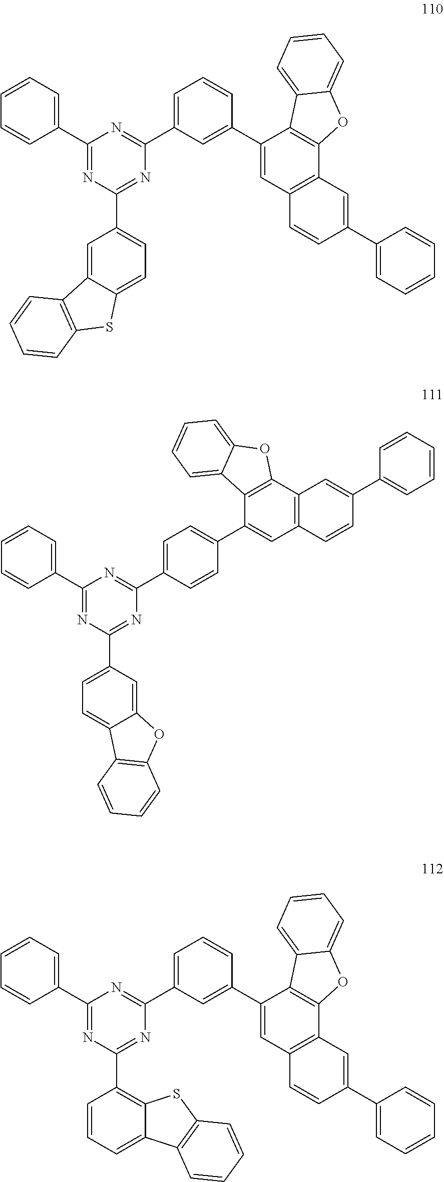 [SMILES: c1ccc(-c2ccc3cc(-c4cccc(-c5nc(-c6ccccc6)nc(-c6ccc7sc8ccccc8c7c6)n5)c4)c4c5ccccc5oc4c3c2)cc1 ; c1ccc(-c2ccc3cc(-c4ccc(-c5nc(-c6ccccc6)nc(-c6ccc7c(c6)oc6ccccc67)n5)cc4)c4c5ccccc5oc4c3c2)cc1 ; c1ccc(-c2ccc3cc(-c4cccc(-c5nc(-c6ccccc6)nc(-c6cccc7c6sc6ccccc67)n5)c4)c4c5ccccc5oc4c3c2)cc1]

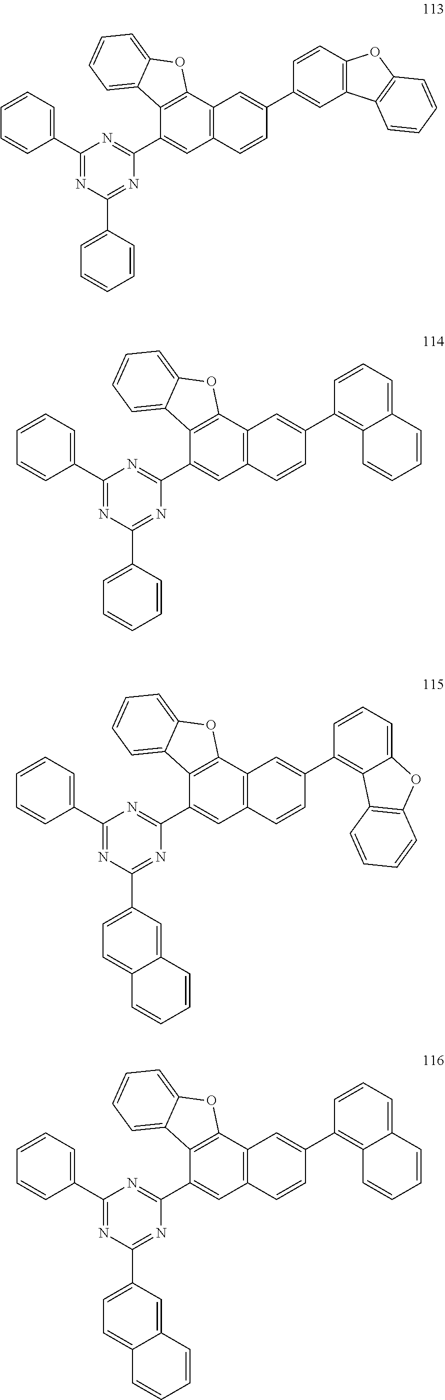

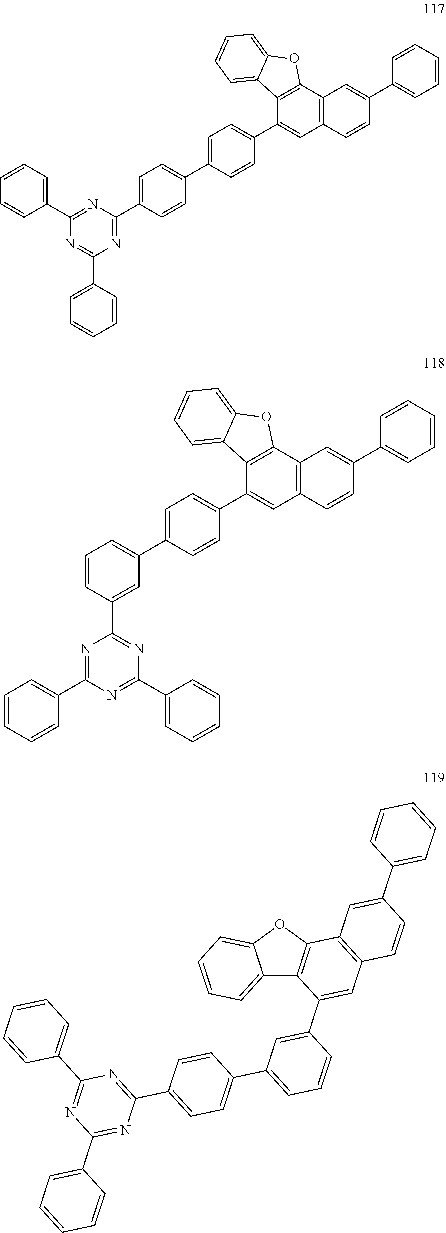 [SMILES: c1ccc(-c2ccc3cc(-c4ccc(-c5ccc(-c6nc(-c7ccccc7)nc(-c7ccccc7)n6)cc5)cc4)c4c5ccccc5oc4c3c2)cc1 ; c1ccc(-c2ccc3cc(-c4ccc(-c5cccc(-c6nc(-c7ccccc7)nc(-c7ccccc7)n6)c5)cc4)c4c5ccccc5oc4c3c2)cc1 ; c1ccc(-c2ccc3cc(-c4cccc(-c5ccc(-c6nc(-c7ccccc7)nc(-c7ccccc7)n6)cc5)c4)c4c5ccccc5oc4c3c2)cc1]

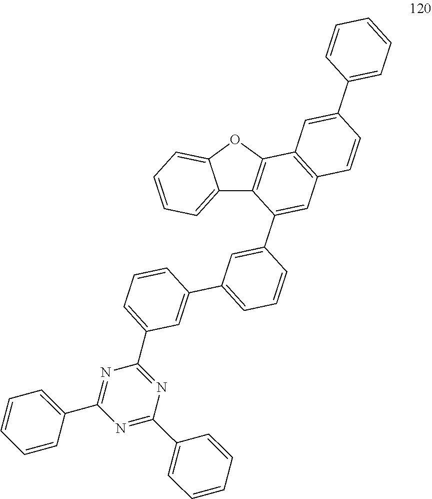 [SMILES: c1ccc(-c2ccc3cc(-c4cccc(-c5cccc(-c6nc(-c7ccccc7)nc(-c7ccccc7)n6)c5)c4)c4c5ccccc5oc4c3c2)cc1]

[chemical structure]

[chemical structure]

[chemical structure]

[chemical structure]

[chemical structure]

[chemical structure]

[chemical structure]

[chemical structure]

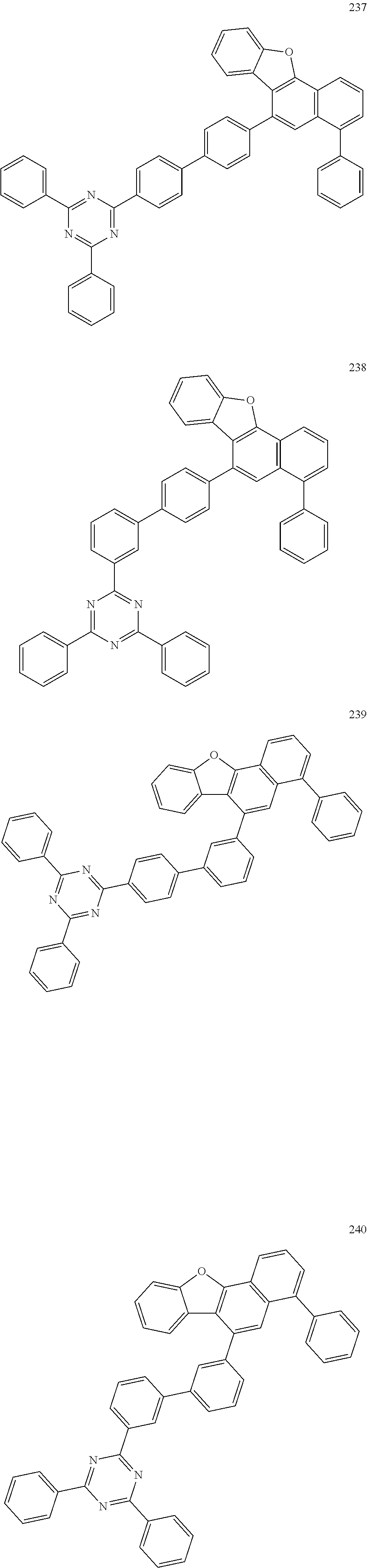 [SMILES: c1ccc(-c2nc(-c3ccccc3)nc(-c3ccc(-c4ccc(-c5cc6c(-c7ccccc7)cccc6c6oc7ccccc7c56)cc4)cc3)n2)cc1 ; c1ccc(-c2nc(-c3ccccc3)nc(-c3cccc(-c4ccc(-c5cc6c(-c7ccccc7)cccc6c6oc7ccccc7c56)cc4)c3)n2)cc1 ; c1ccc(-c2nc(-c3ccccc3)nc(-c3ccc(-c4cccc(-c5cc6c(-c7ccccc7)cccc6c6oc7ccccc7c56)c4)cc3)n2)cc1 ; c1ccc(-c2nc(-c3ccccc3)nc(-c3cccc(-c4cccc(-c5cc6c(-c7ccccc7)cccc6c6oc7ccccc7c56)c4)c3)n2)cc1]

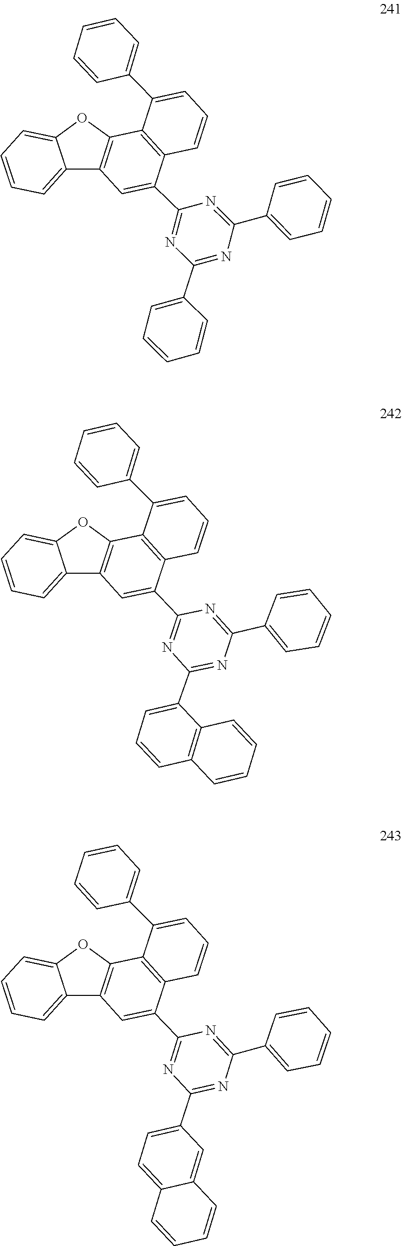 [SMILES: c1ccc(-c2nc(-c3ccccc3)nc(-c3cc4c5ccccc5oc4c4c(-c5ccccc5)cccc34)n2)cc1 ; c1ccc(-c2nc(-c3cccc4ccccc34)nc(-c3cc4c5ccccc5oc4c4c(-c5ccccc5)cccc34)n2)cc1 ; c1ccc(-c2nc(-c3ccc4ccccc4c3)nc(-c3cc4c5ccccc5oc4c4c(-c5ccccc5)cccc34)n2)cc1]

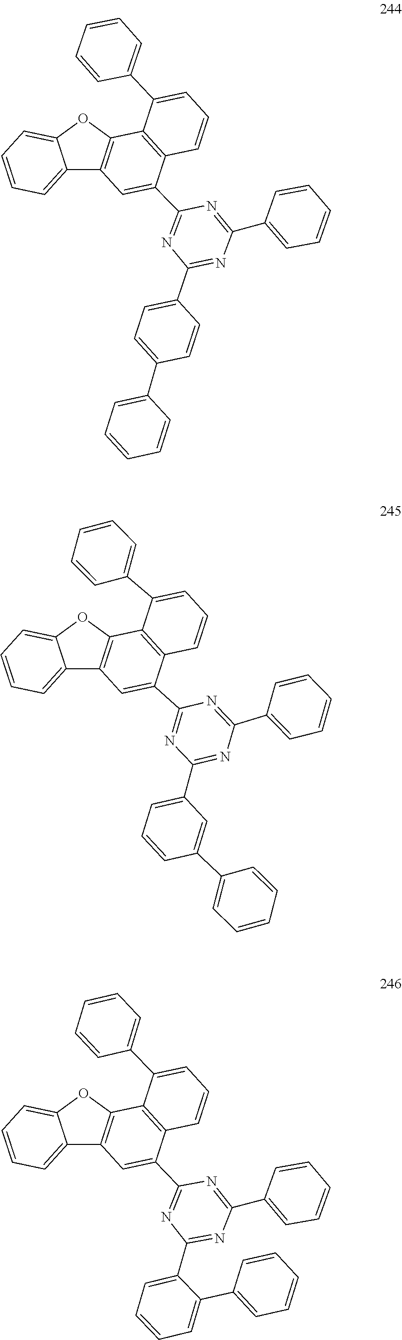 [SMILES: c1ccc(-c2ccc(-c3nc(-c4ccccc4)nc(-c4cc5c6ccccc6oc5c5c(-c6ccccc6)cccc45)n3)cc2)cc1 ; c1ccc(-c2cccc(-c3nc(-c4ccccc4)nc(-c4cc5c6ccccc6oc5c5c(-c6ccccc6)cccc45)n3)c2)cc1 ; c1ccc(-c2nc(-c3ccccc3-c3ccccc3)nc(-c3cc4c5ccccc5oc4c4c(-c5ccccc5)cccc34)n2)cc1]

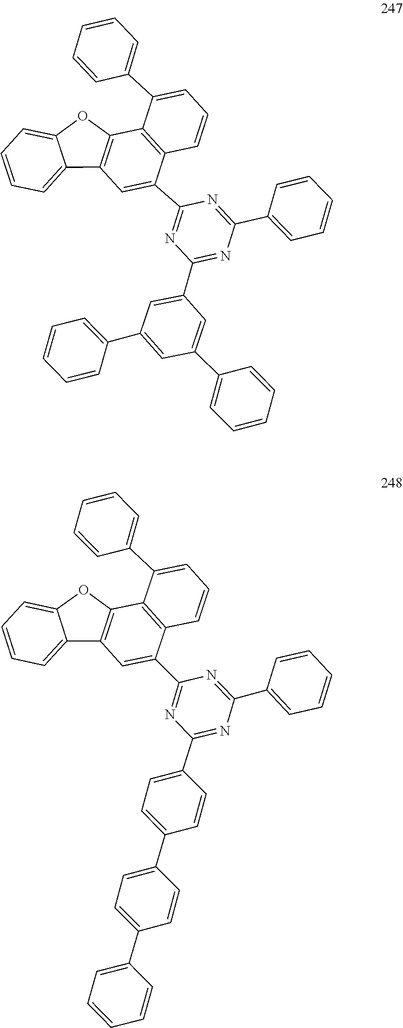 [SMILES: c1ccc(-c2cc(-c3ccccc3)cc(-c3nc(-c4ccccc4)nc(-c4cc5c6ccccc6oc5c5c(-c6ccccc6)cccc45)n3)c2)cc1 ; c1ccc(-c2ccc(-c3ccc(-c4nc(-c5ccccc5)nc(-c5cc6c7ccccc7oc6c6c(-c7ccccc7)cccc56)n4)cc3)cc2)cc1]

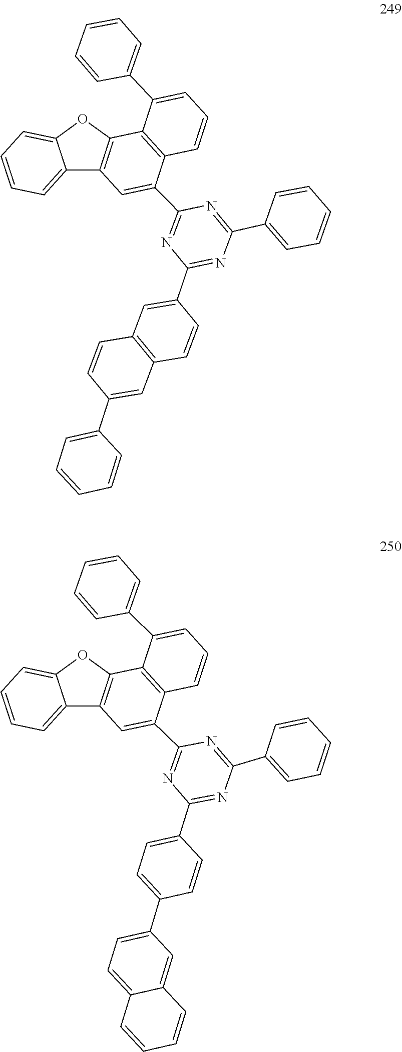 [SMILES: c1ccc(-c2ccc3cc(-c4nc(-c5ccccc5)nc(-c5cc6c7ccccc7oc6c6c(-c7ccccc7)cccc56)n4)ccc3c2)cc1 ; c1ccc(-c2nc(-c3ccc(-c4ccc5ccccc5c4)cc3)nc(-c3cc4c5ccccc5oc4c4c(-c5ccccc5)cccc34)n2)cc1]

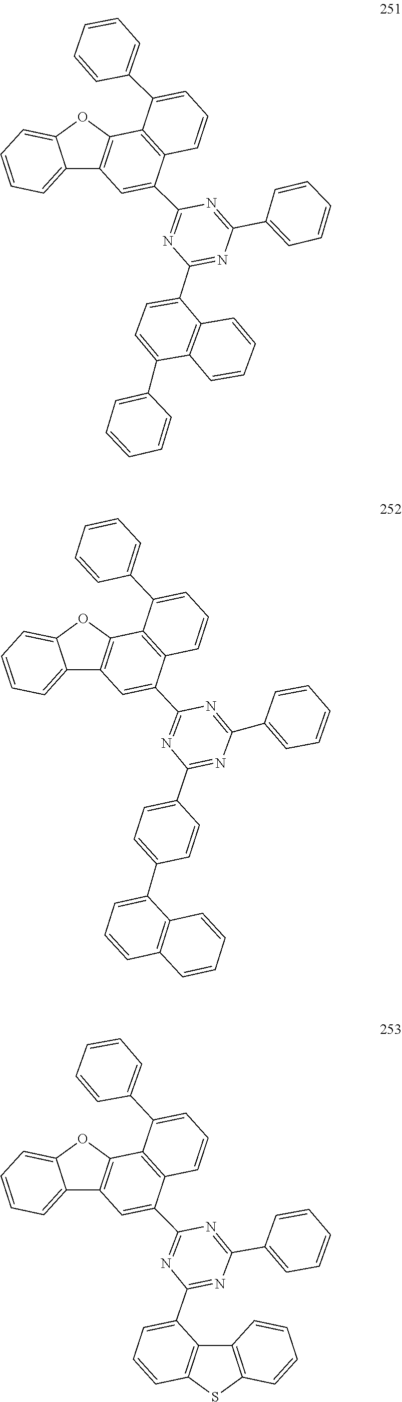 [SMILES: c1ccc(-c2nc(-c3ccc(-c4ccccc4)c4ccccc34)nc(-c3cc4c5ccccc5oc4c4c(-c5ccccc5)cccc34)n2)cc1 ; c1ccc(-c2nc(-c3ccc(-c4cccc5ccccc45)cc3)nc(-c3cc4c5ccccc5oc4c4c(-c5ccccc5)cccc34)n2)cc1 ; c1ccc(-c2nc(-c3cc4c5ccccc5oc4c4c(-c5ccccc5)cccc34)nc(-c3cccc4sc5ccccc5c34)n2)cc1]

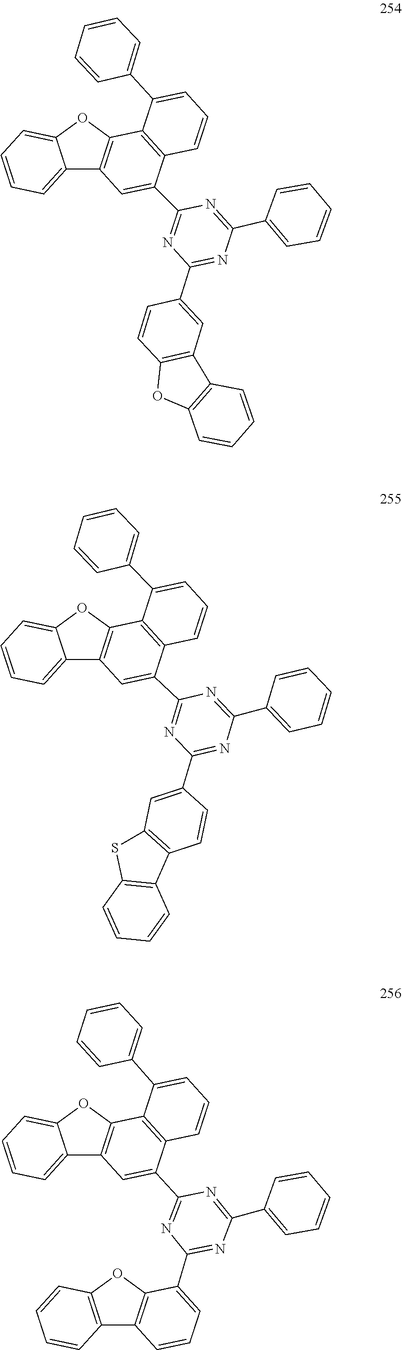 [SMILES: c1ccc(-c2nc(-c3ccc4oc5ccccc5c4c3)nc(-c3cc4c5ccccc5oc4c4c(-c5ccccc5)cccc34)n2)cc1 ; c1ccc(-c2nc(-c3ccc4c(c3)sc3ccccc34)nc(-c3cc4c5ccccc5oc4c4c(-c5ccccc5)cccc34)n2)cc1 ; c1ccc(-c2nc(-c3cc4c5ccccc5oc4c4c(-c5ccccc5)cccc34)nc(-c3cccc4c3oc3ccccc34)n2)cc1]

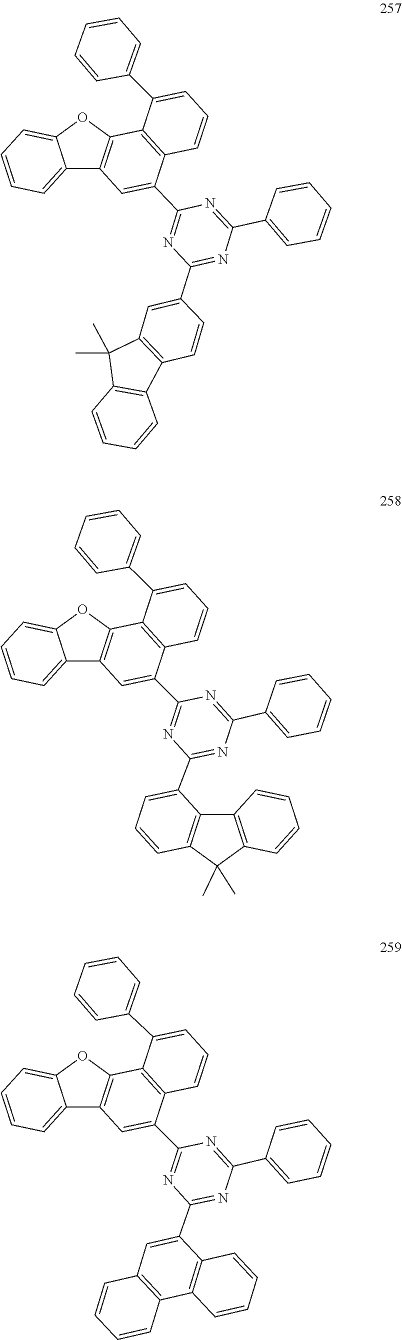 [SMILES: CC1(C)c2ccccc2-c2ccc(-c3nc(-c4ccccc4)nc(-c4cc5c6ccccc6oc5c5c(-c6ccccc6)cccc45)n3)cc21 ; CC1(C)c2ccccc2-c2c(-c3nc(-c4ccccc4)nc(-c4cc5c6ccccc6oc5c5c(-c6ccccc6)cccc45)n3)cccc21 ; c1ccc(-c2nc(-c3cc4ccccc4c4ccccc34)nc(-c3cc4c5ccccc5oc4c4c(-c5ccccc5)cccc34)n2)cc1]

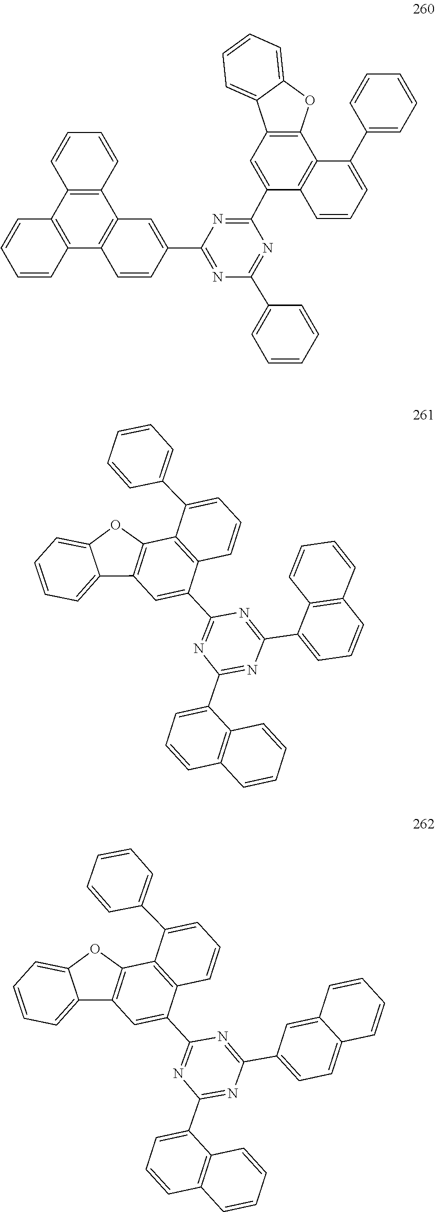 [SMILES: c1ccc(-c2nc(-c3ccc4c5ccccc5c5ccccc5c4c3)nc(-c3cc4c5ccccc5oc4c4c(-c5ccccc5)cccc34)n2)cc1 ; c1ccc(-c2cccc3c(-c4nc(-c5cccc6ccccc56)nc(-c5cccc6ccccc56)n4)cc4c5ccccc5oc4c23)cc1 ; c1ccc(-c2cccc3c(-c4nc(-c5ccc6ccccc6c5)nc(-c5cccc6ccccc56)n4)cc4c5ccccc5oc4c23)cc1]

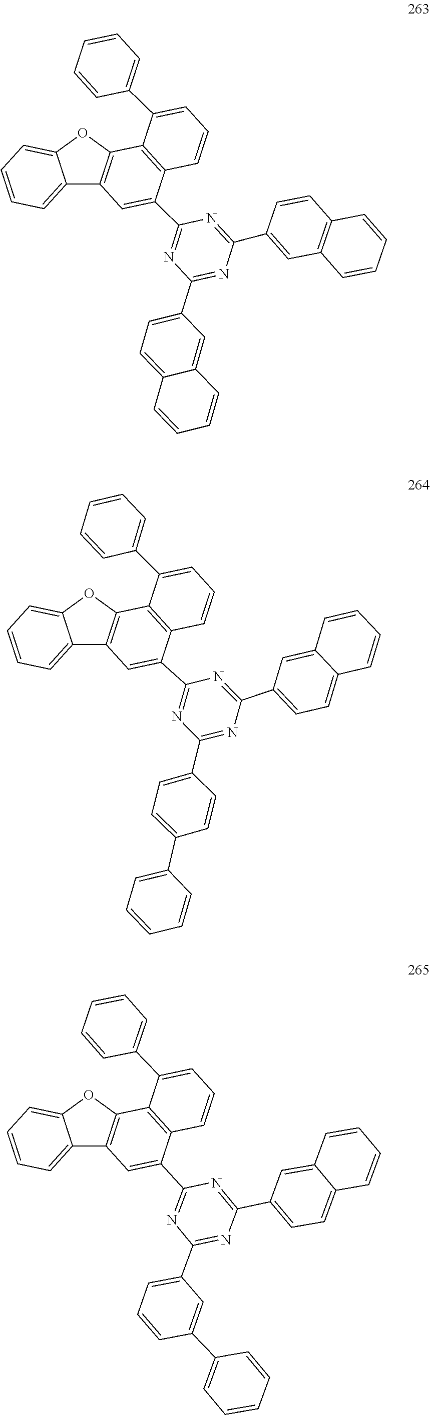 [SMILES: c1ccc(-c2cccc3c(-c4nc(-c5ccc6ccccc6c5)nc(-c5ccc6ccccc6c5)n4)cc4c5ccccc5oc4c23)cc1 ; c1ccc(-c2ccc(-c3nc(-c4ccc5ccccc5c4)nc(-c4cc5c6ccccc6oc5c5c(-c6ccccc6)cccc45)n3)cc2)cc1 ; c1ccc(-c2cccc(-c3nc(-c4ccc5ccccc5c4)nc(-c4cc5c6ccccc6oc5c5c(-c6ccccc6)cccc45)n3)c2)cc1]

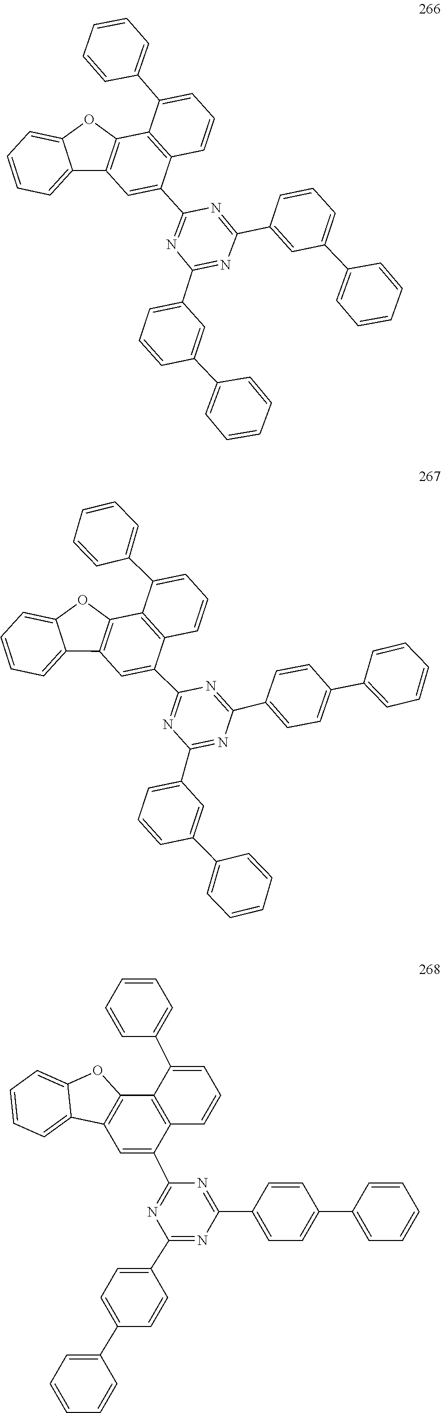 [SMILES: c1ccc(-c2cccc(-c3nc(-c4cccc(-c5ccccc5)c4)nc(-c4cc5c6ccccc6oc5c5c(-c6ccccc6)cccc45)n3)c2)cc1 ; c1ccc(-c2ccc(-c3nc(-c4cccc(-c5ccccc5)c4)nc(-c4cc5c6ccccc6oc5c5c(-c6ccccc6)cccc45)n3)cc2)cc1 ; c1ccc(-c2ccc(-c3nc(-c4ccc(-c5ccccc5)cc4)nc(-c4cc5c6ccccc6oc5c5c(-c6ccccc6)cccc45)n3)cc2)cc1]

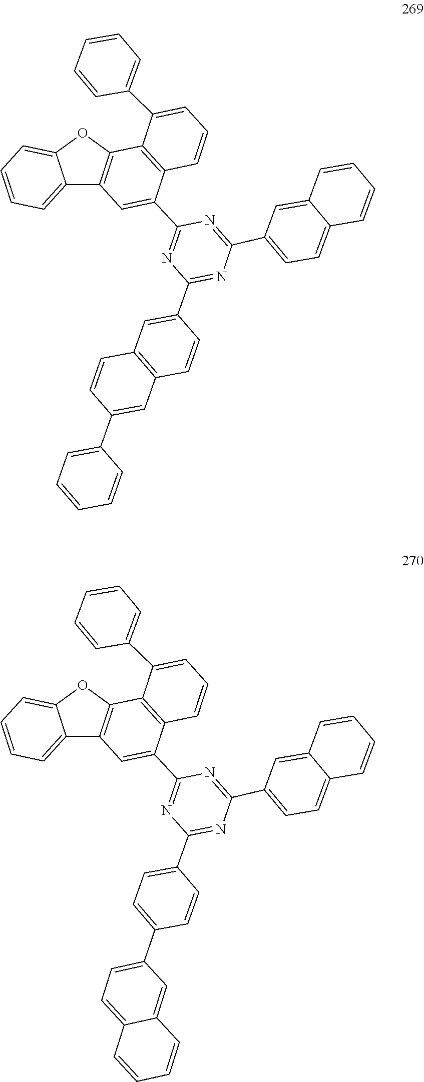 [SMILES: c1ccc(-c2ccc3cc(-c4nc(-c5ccc6ccccc6c5)nc(-c5cc6c7ccccc7oc6c6c(-c7ccccc7)cccc56)n4)ccc3c2)cc1 ; c1ccc(-c2cccc3c(-c4nc(-c5ccc(-c6ccc7ccccc7c6)cc5)nc(-c5ccc6ccccc6c5)n4)cc4c5ccccc5oc4c23)cc1]

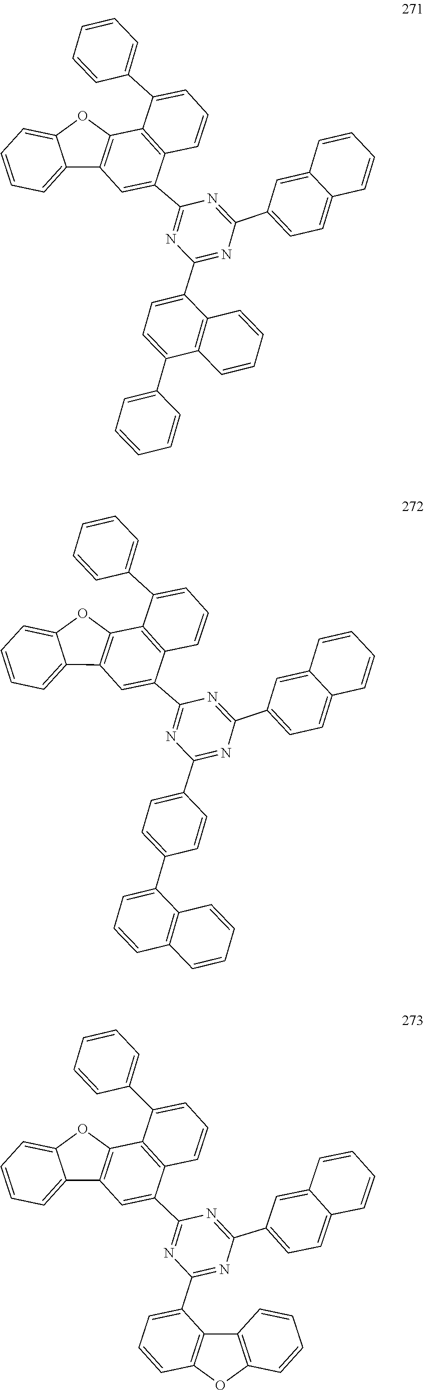 [SMILES: c1ccc(-c2ccc(-c3nc(-c4ccc5ccccc5c4)nc(-c4cc5c6ccccc6oc5c5c(-c6ccccc6)cccc45)n3)c3ccccc23)cc1 ; c1ccc(-c2cccc3c(-c4nc(-c5ccc(-c6cccc7ccccc67)cc5)nc(-c5ccc6ccccc6c5)n4)cc4c5ccccc5oc4c23)cc1 ; c1ccc(-c2cccc3c(-c4nc(-c5ccc6ccccc6c5)nc(-c5cccc6oc7ccccc7c56)n4)cc4c5ccccc5oc4c23)cc1]

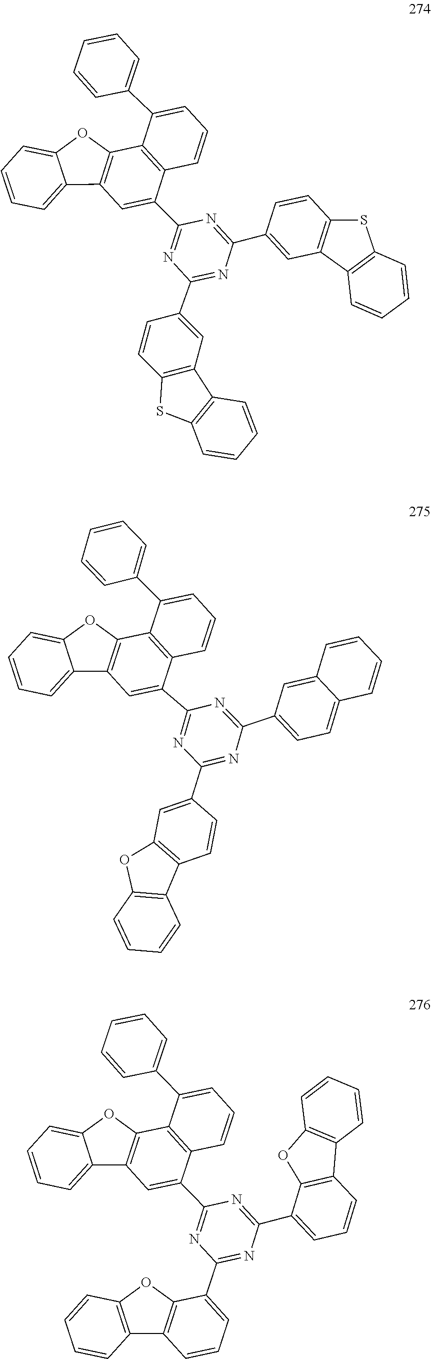 [SMILES: c1ccc(-c2cccc3c(-c4nc(-c5ccc6sc7ccccc7c6c5)nc(-c5ccc6sc7ccccc7c6c5)n4)cc4c5ccccc5oc4c23)cc1 ; c1ccc(-c2cccc3c(-c4nc(-c5ccc6ccccc6c5)nc(-c5ccc6c(c5)oc5ccccc56)n4)cc4c5ccccc5oc4c23)cc1 ; c1ccc(-c2cccc3c(-c4nc(-c5cccc6c5oc5ccccc56)nc(-c5cccc6c5oc5ccccc56)n4)cc4c5ccccc5oc4c23)cc1]

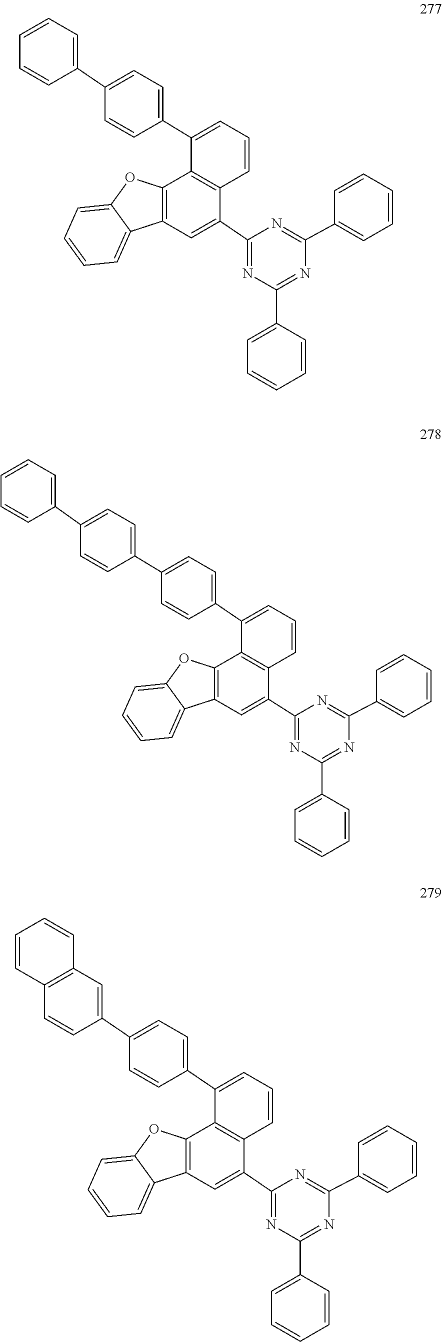 [SMILES: c1ccc(-c2ccc(-c3cccc4c(-c5nc(-c6ccccc6)nc(-c6ccccc6)n5)cc5c6ccccc6oc5c34)cc2)cc1 ; c1ccc(-c2ccc(-c3ccc(-c4cccc5c(-c6nc(-c7ccccc7)nc(-c7ccccc7)n6)cc6c7ccccc7oc6c45)cc3)cc2)cc1 ; c1ccc(-c2nc(-c3ccccc3)nc(-c3cc4c5ccccc5oc4c4c(-c5ccc(-c6ccc7ccccc7c6)cc5)cccc34)n2)cc1]

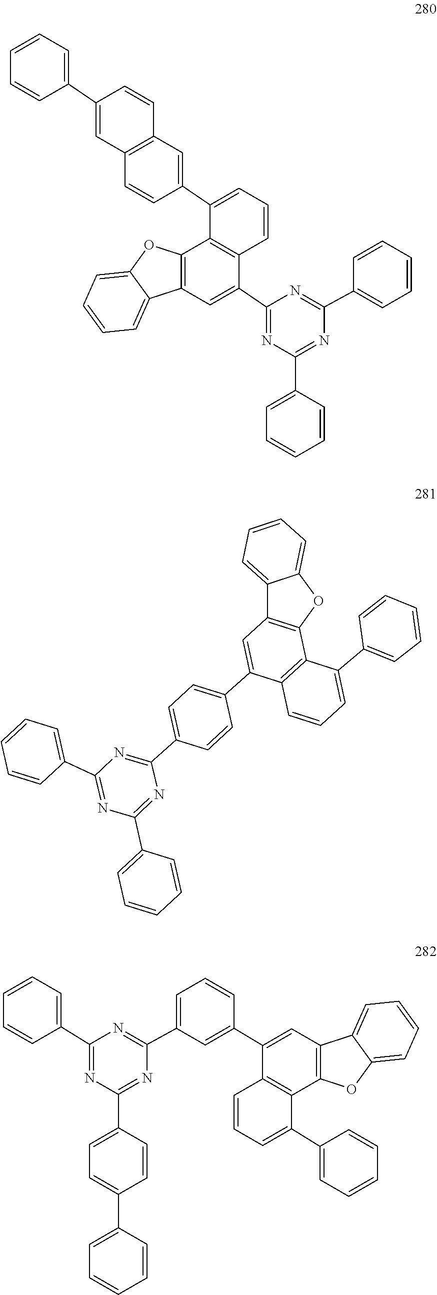 [SMILES: c1ccc(-c2ccc3cc(-c4cccc5c(-c6nc(-c7ccccc7)nc(-c7ccccc7)n6)cc6c7ccccc7oc6c45)ccc3c2)cc1 ; c1ccc(-c2nc(-c3ccccc3)nc(-c3ccc(-c4cc5c6ccccc6oc5c5c(-c6ccccc6)cccc45)cc3)n2)cc1 ; c1ccc(-c2ccc(-c3nc(-c4ccccc4)nc(-c4cccc(-c5cc6c7ccccc7oc6c6c(-c7ccccc7)cccc56)c4)n3)cc2)cc1]

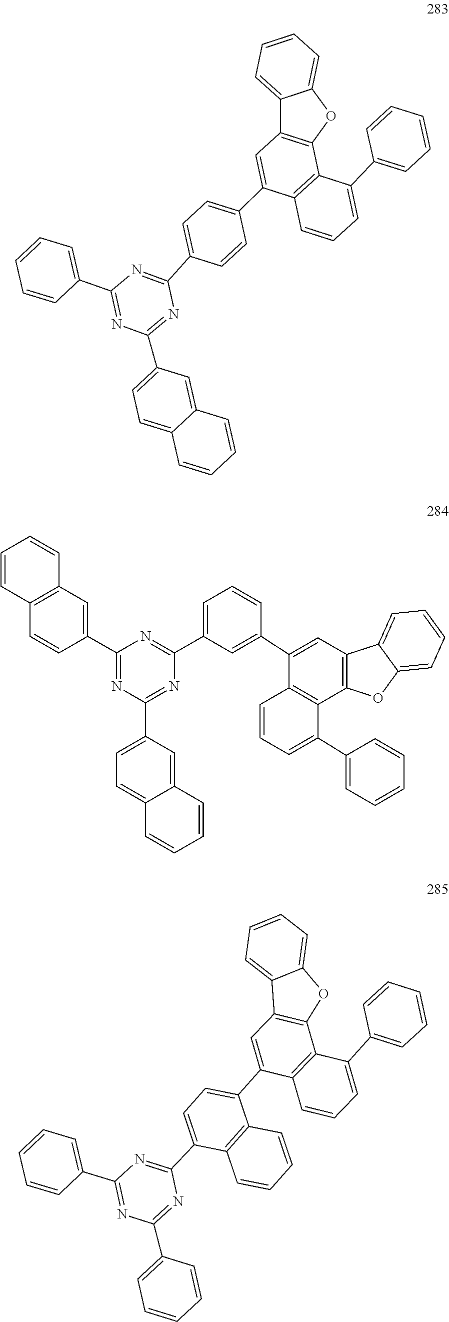 [SMILES: c1ccc(-c2nc(-c3ccc(-c4cc5c6ccccc6oc5c5c(-c6ccccc6)cccc45)cc3)nc(-c3ccc4ccccc4c3)n2)cc1 ; c1ccc(-c2cccc3c(-c4cccc(-c5nc(-c6ccc7ccccc7c6)nc(-c6ccc7ccccc7c6)n5)c4)cc4c5ccccc5oc4c23)cc1 ; c1ccc(-c2nc(-c3ccccc3)nc(-c3ccc(-c4cc5c6ccccc6oc5c5c(-c6ccccc6)cccc45)c4ccccc34)n2)cc1]

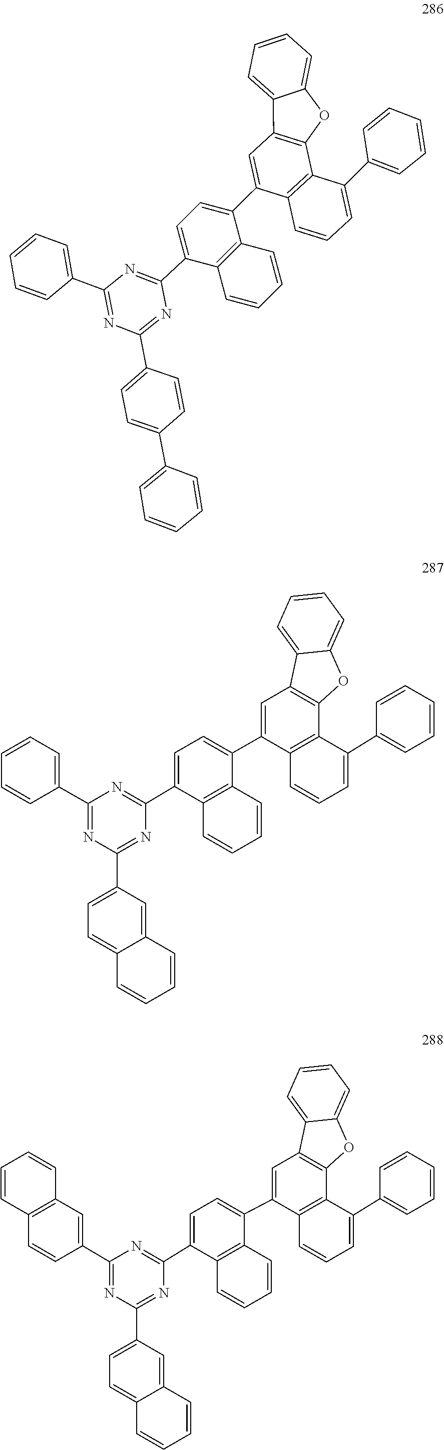 [SMILES: c1ccc(-c2ccc(-c3nc(-c4ccccc4)nc(-c4ccc(-c5cc6c7ccccc7oc6c6c(-c7ccccc7)cccc56)c5ccccc45)n3)cc2)cc1 ; c1ccc(-c2nc(-c3ccc4ccccc4c3)nc(-c3ccc(-c4cc5c6ccccc6oc5c5c(-c6ccccc6)cccc45)c4ccccc34)n2)cc1 ; c1ccc(-c2cccc3c(-c4ccc(-c5nc(-c6ccc7ccccc7c6)nc(-c6ccc7ccccc7c6)n5)c5ccccc45)cc4c5ccccc5oc4c23)cc1]

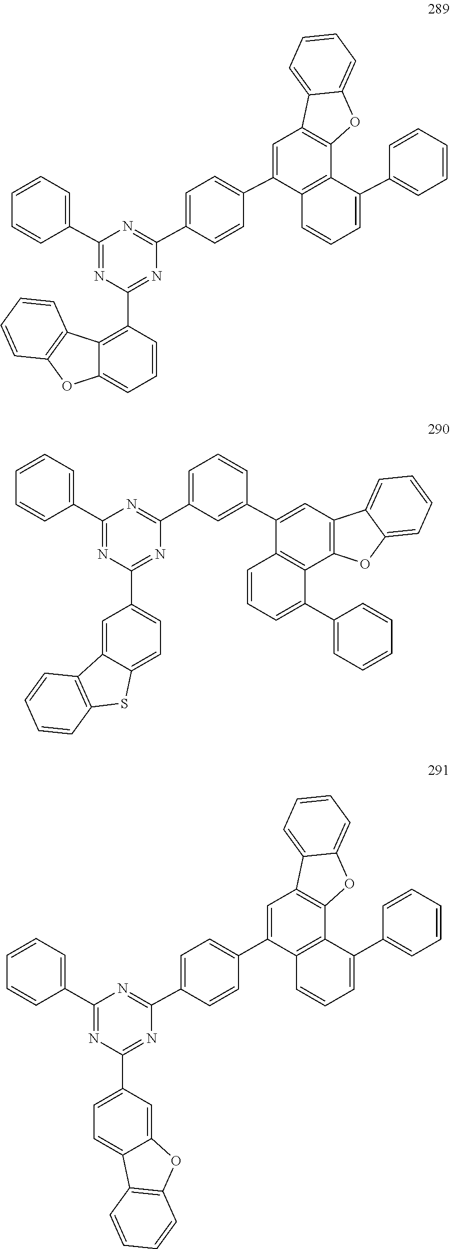 [SMILES: c1ccc(-c2nc(-c3ccc(-c4cc5c6ccccc6oc5c5c(-c6ccccc6)cccc45)cc3)nc(-c3cccc4oc5ccccc5c34)n2)cc1 ; c1ccc(-c2nc(-c3cccc(-c4cc5c6ccccc6oc5c5c(-c6ccccc6)cccc45)c3)nc(-c3ccc4sc5ccccc5c4c3)n2)cc1 ; c1ccc(-c2nc(-c3ccc(-c4cc5c6ccccc6oc5c5c(-c6ccccc6)cccc45)cc3)nc(-c3ccc4c(c3)oc3ccccc34)n2)cc1]

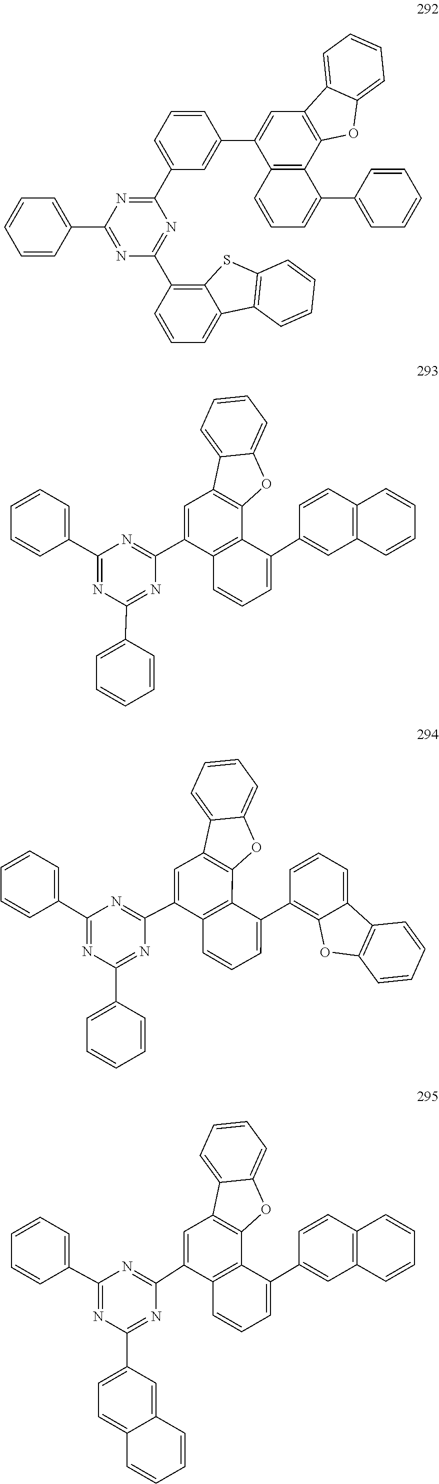 [SMILES: c1ccc(-c2nc(-c3cccc(-c4cc5c6ccccc6oc5c5c(-c6ccccc6)cccc45)c3)nc(-c3cccc4c3sc3ccccc34)n2)cc1 ; c1ccc(-c2nc(-c3ccccc3)nc(-c3cc4c5ccccc5oc4c4c(-c5ccc6ccccc6c5)cccc34)n2)cc1 ; c1ccc(-c2nc(-c3ccccc3)nc(-c3cc4c5ccccc5oc4c4c(-c5cccc6c5oc5ccccc56)cccc34)n2)cc1 ; c1ccc(-c2nc(-c3ccc4ccccc4c3)nc(-c3cc4c5ccccc5oc4c4c(-c5ccc6ccccc6c5)cccc34)n2)cc1]

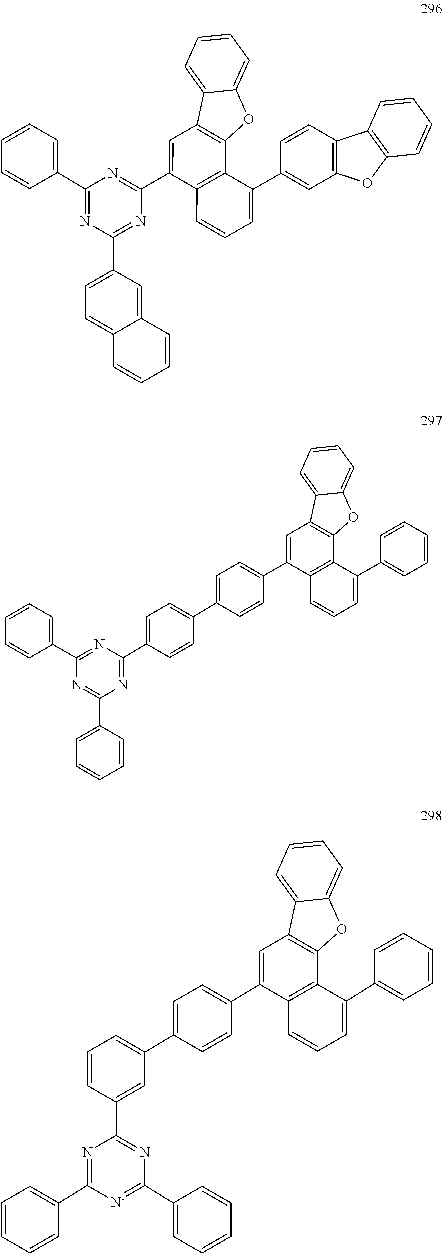

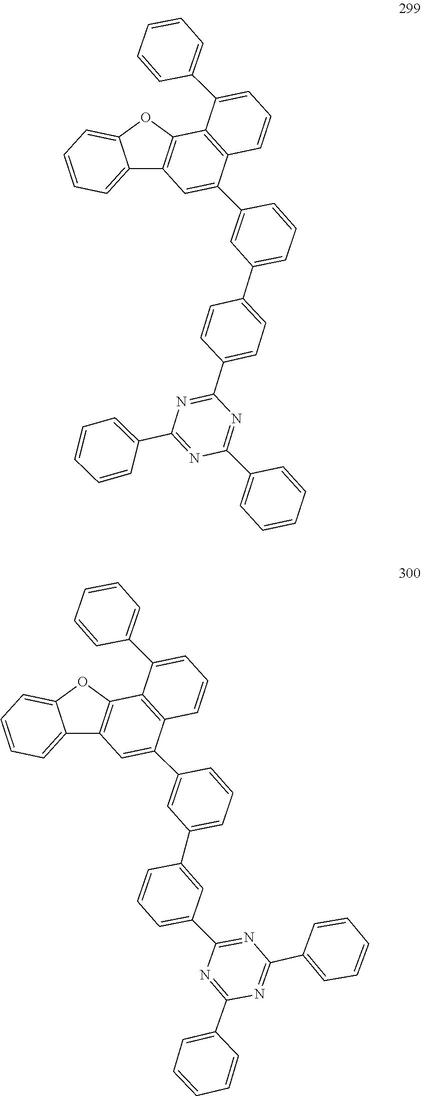 [SMILES: c1ccc(-c2nc(-c3ccccc3)nc(-c3ccc(-c4cccc(-c5cc6c7ccccc7oc6c6c(-c7ccccc7)cccc56)c4)cc3)n2)cc1 ; c1ccc(-c2nc(-c3ccccc3)nc(-c3cccc(-c4cccc(-c5cc6c7ccccc7oc6c6c(-c7ccccc7)cccc56)c4)c3)n2)cc1]

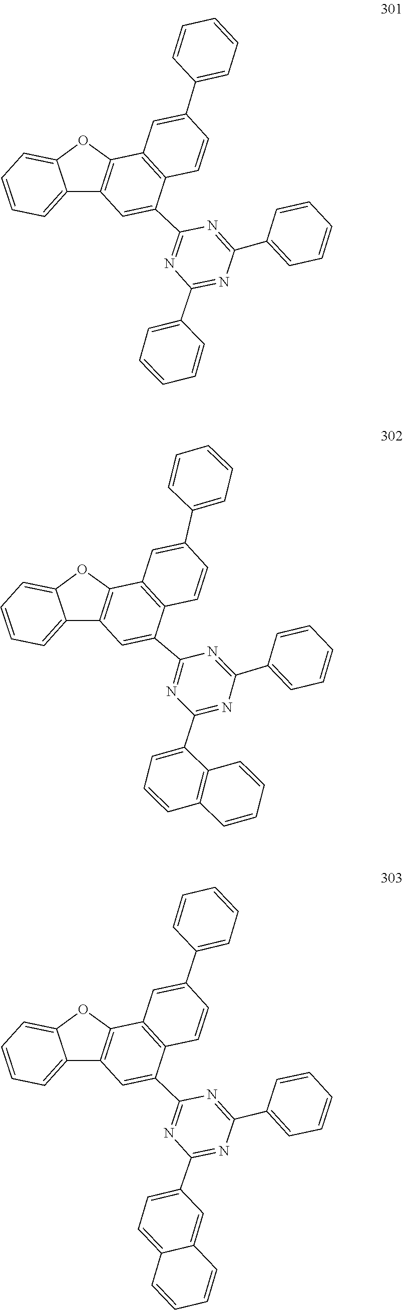 [SMILES: c1ccc(-c2ccc3c(-c4nc(-c5ccccc5)nc(-c5ccccc5)n4)cc4c5ccccc5oc4c3c2)cc1 ; c1ccc(-c2ccc3c(-c4nc(-c5ccccc5)nc(-c5cccc6ccccc56)n4)cc4c5ccccc5oc4c3c2)cc1 ; c1ccc(-c2ccc3c(-c4nc(-c5ccccc5)nc(-c5ccc6ccccc6c5)n4)cc4c5ccccc5oc4c3c2)cc1]

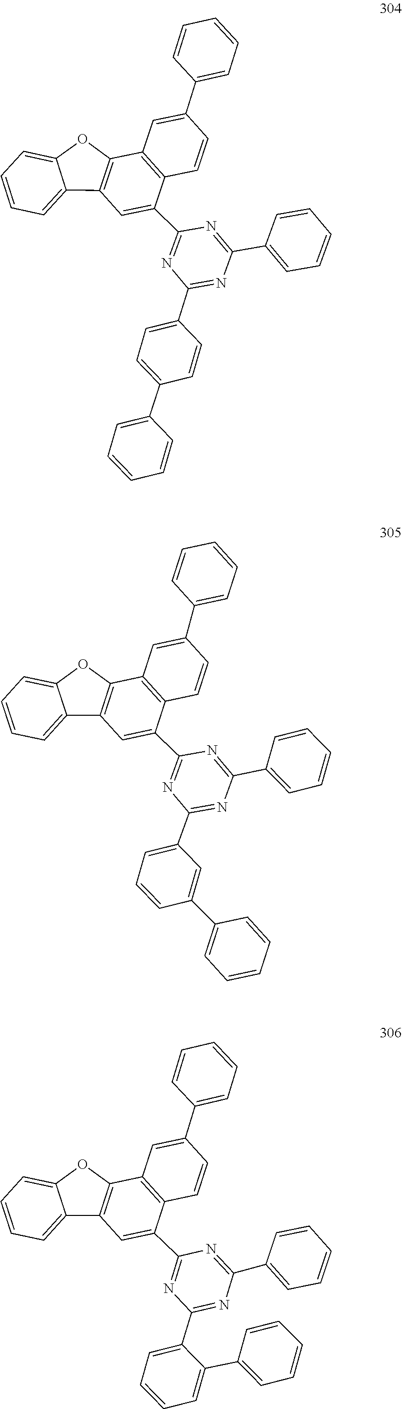 [SMILES: c1ccc(-c2ccc(-c3nc(-c4ccccc4)nc(-c4cc5c6ccccc6oc5c5cc(-c6ccccc6)ccc45)n3)cc2)cc1 ; c1ccc(-c2cccc(-c3nc(-c4ccccc4)nc(-c4cc5c6ccccc6oc5c5cc(-c6ccccc6)ccc45)n3)c2)cc1 ; c1ccc(-c2ccc3c(-c4nc(-c5ccccc5)nc(-c5ccccc5-c5ccccc5)n4)cc4c5ccccc5oc4c3c2)cc1]

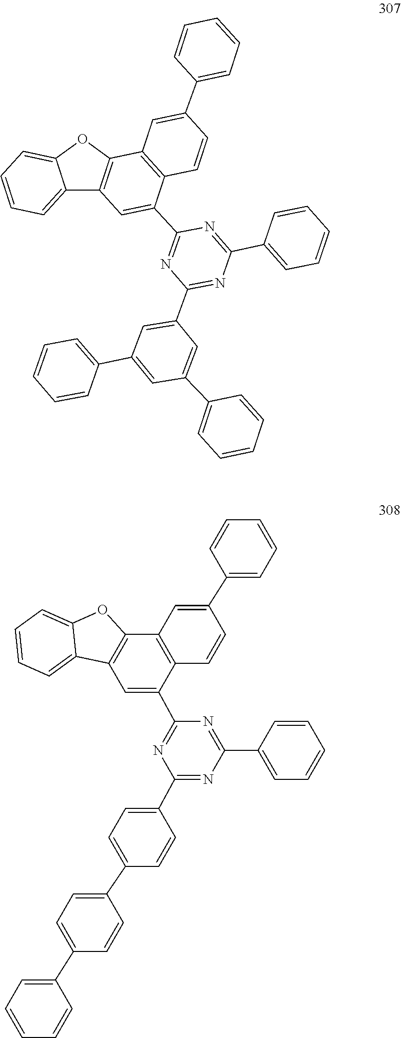 [SMILES: c1ccc(-c2cc(-c3ccccc3)cc(-c3nc(-c4ccccc4)nc(-c4cc5c6ccccc6oc5c5cc(-c6ccccc6)ccc45)n3)c2)cc1 ; c1ccc(-c2ccc(-c3ccc(-c4nc(-c5ccccc5)nc(-c5cc6c7ccccc7oc6c6cc(-c7ccccc7)ccc56)n4)cc3)cc2)cc1]

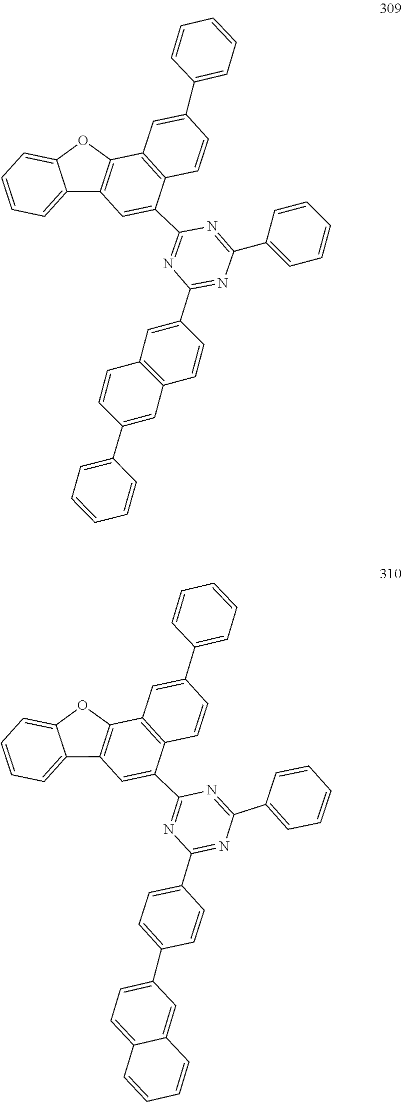 [SMILES: c1ccc(-c2ccc3cc(-c4nc(-c5ccccc5)nc(-c5cc6c7ccccc7oc6c6cc(-c7ccccc7)ccc56)n4)ccc3c2)cc1 ; c1ccc(-c2ccc3c(-c4nc(-c5ccccc5)nc(-c5ccc(-c6ccc7ccccc7c6)cc5)n4)cc4c5ccccc5oc4c3c2)cc1]

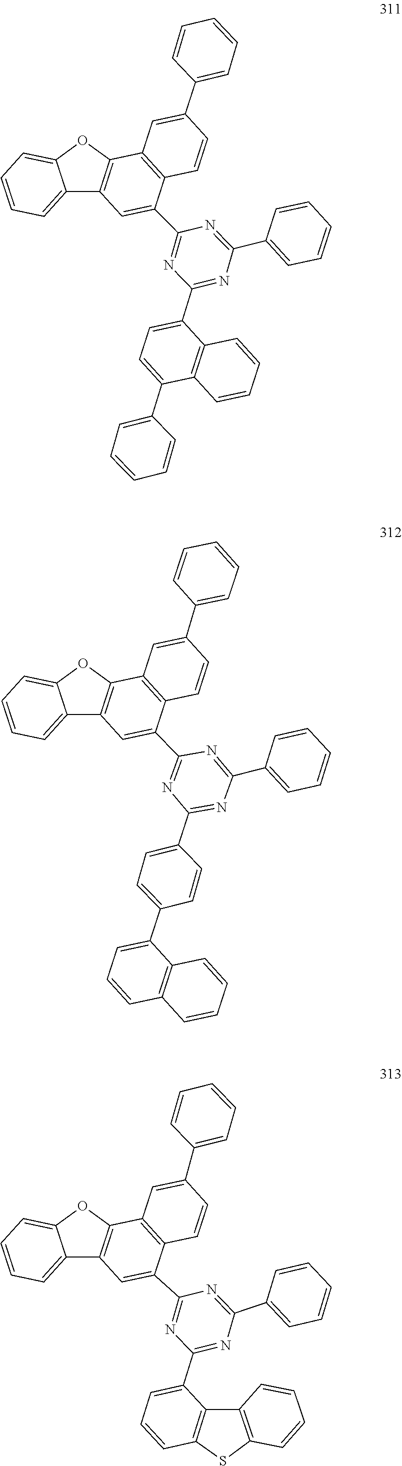 [SMILES: c1ccc(-c2ccc3c(-c4nc(-c5ccccc5)nc(-c5ccc(-c6ccccc6)c6ccccc56)n4)cc4c5ccccc5oc4c3c2)cc1 ; c1ccc(-c2ccc3c(-c4nc(-c5ccccc5)nc(-c5ccc(-c6cccc7ccccc67)cc5)n4)cc4c5ccccc5oc4c3c2)cc1 ; c1ccc(-c2ccc3c(-c4nc(-c5ccccc5)nc(-c5cccc6sc7ccccc7c56)n4)cc4c5ccccc5oc4c3c2)cc1]

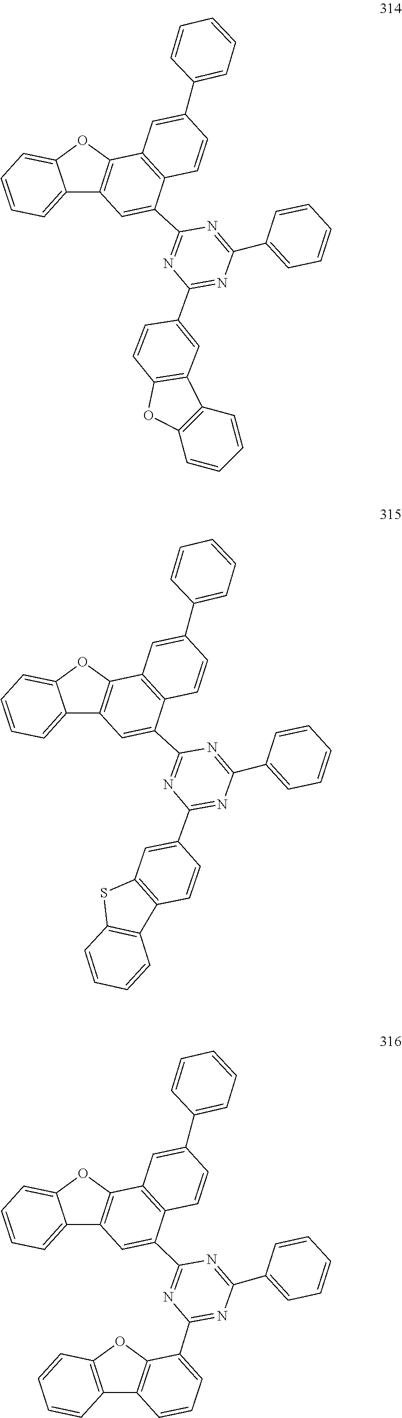 [SMILES: c1ccc(-c2ccc3c(-c4nc(-c5ccccc5)nc(-c5ccc6oc7ccccc7c6c5)n4)cc4c5ccccc5oc4c3c2)cc1 ; c1ccc(-c2ccc3c(-c4nc(-c5ccccc5)nc(-c5ccc6c(c5)sc5ccccc56)n4)cc4c5ccccc5oc4c3c2)cc1 ; c1ccc(-c2ccc3c(-c4nc(-c5ccccc5)nc(-c5cccc6c5oc5ccccc56)n4)cc4c5ccccc5oc4c3c2)cc1]

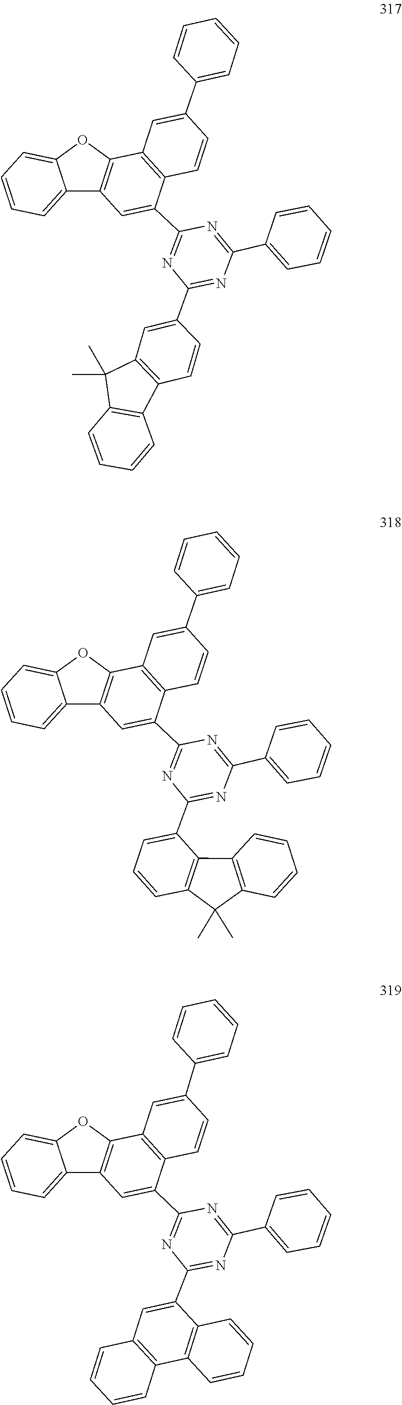 [SMILES: CC1(C)c2ccccc2-c2ccc(-c3nc(-c4ccccc4)nc(-c4cc5c6ccccc6oc5c5cc(-c6ccccc6)ccc45)n3)cc21 ; CC1(C)c2ccccc2-c2c(-c3nc(-c4ccccc4)nc(-c4cc5c6ccccc6oc5c5cc(-c6ccccc6)ccc45)n3)cccc21 ; c1ccc(-c2ccc3c(-c4nc(-c5ccccc5)nc(-c5cc6ccccc6c6ccccc56)n4)cc4c5ccccc5oc4c3c2)cc1]

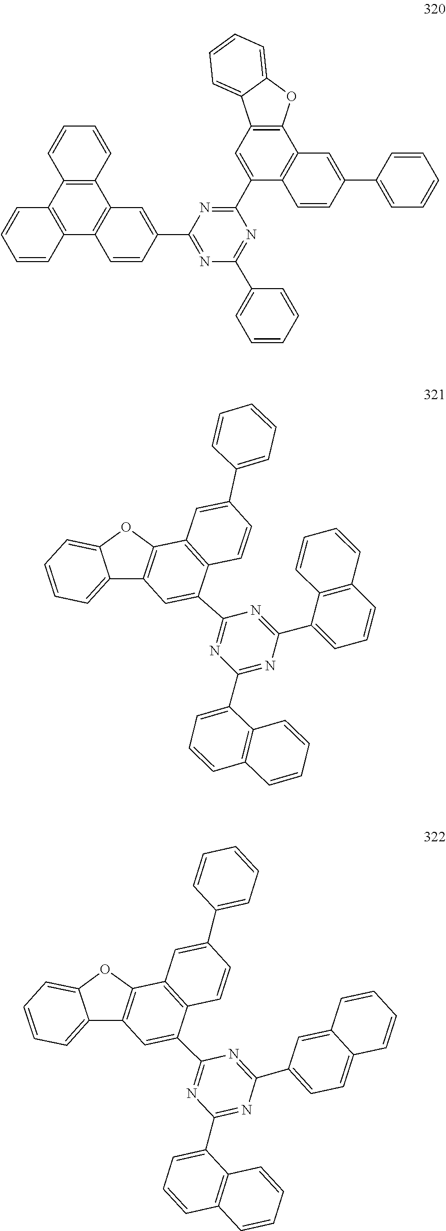 [SMILES: c1ccc(-c2ccc3c(-c4nc(-c5ccccc5)nc(-c5ccc6c7ccccc7c7ccccc7c6c5)n4)cc4c5ccccc5oc4c3c2)cc1 ; c1ccc(-c2ccc3c(-c4nc(-c5cccc6ccccc56)nc(-c5cccc6ccccc56)n4)cc4c5ccccc5oc4c3c2)cc1 ; c1ccc(-c2ccc3c(-c4nc(-c5ccc6ccccc6c5)nc(-c5cccc6ccccc56)n4)cc4c5ccccc5oc4c3c2)cc1]

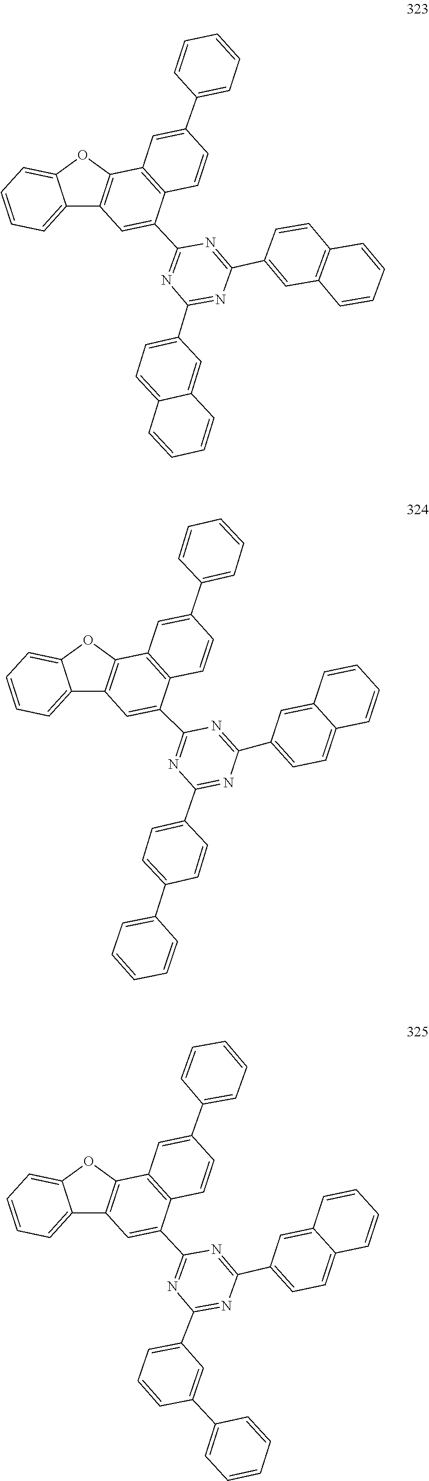 [SMILES: c1ccc(-c2ccc3c(-c4nc(-c5ccc6ccccc6c5)nc(-c5ccc6ccccc6c5)n4)cc4c5ccccc5oc4c3c2)cc1 ; c1ccc(-c2ccc(-c3nc(-c4ccc5ccccc5c4)nc(-c4cc5c6ccccc6oc5c5cc(-c6ccccc6)ccc45)n3)cc2)cc1 ; c1ccc(-c2cccc(-c3nc(-c4ccc5ccccc5c4)nc(-c4cc5c6ccccc6oc5c5cc(-c6ccccc6)ccc45)n3)c2)cc1]

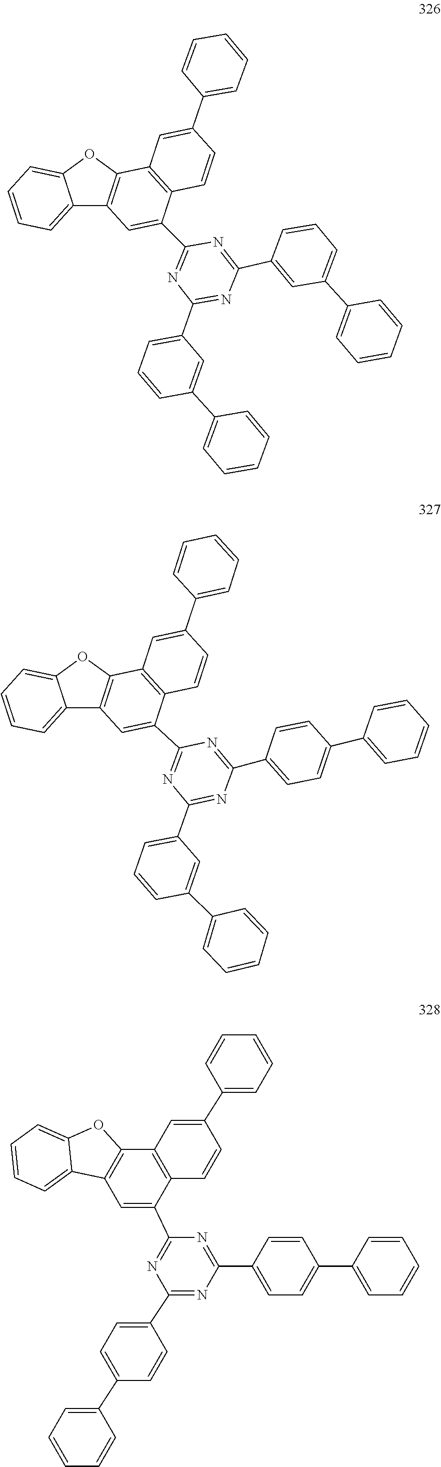 [SMILES: c1ccc(-c2cccc(-c3nc(-c4cccc(-c5ccccc5)c4)nc(-c4cc5c6ccccc6oc5c5cc(-c6ccccc6)ccc45)n3)c2)cc1 ; c1ccc(-c2ccc(-c3nc(-c4cccc(-c5ccccc5)c4)nc(-c4cc5c6ccccc6oc5c5cc(-c6ccccc6)ccc45)n3)cc2)cc1 ; c1ccc(-c2ccc(-c3nc(-c4ccc(-c5ccccc5)cc4)nc(-c4cc5c6ccccc6oc5c5cc(-c6ccccc6)ccc45)n3)cc2)cc1]

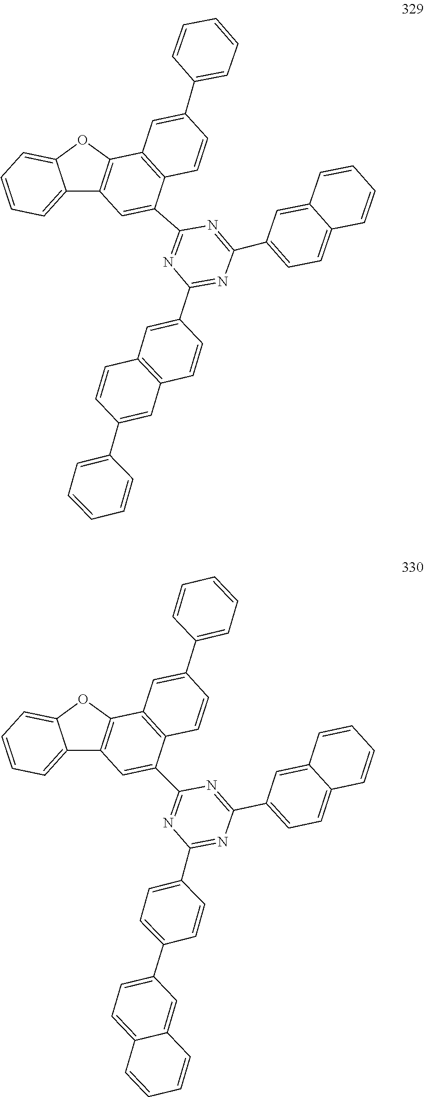 [SMILES: c1ccc(-c2ccc3cc(-c4nc(-c5ccc6ccccc6c5)nc(-c5cc6c7ccccc7oc6c6cc(-c7ccccc7)ccc56)n4)ccc3c2)cc1 ; c1ccc(-c2ccc3c(-c4nc(-c5ccc(-c6ccc7ccccc7c6)cc5)nc(-c5ccc6ccccc6c5)n4)cc4c5ccccc5oc4c3c2)cc1]

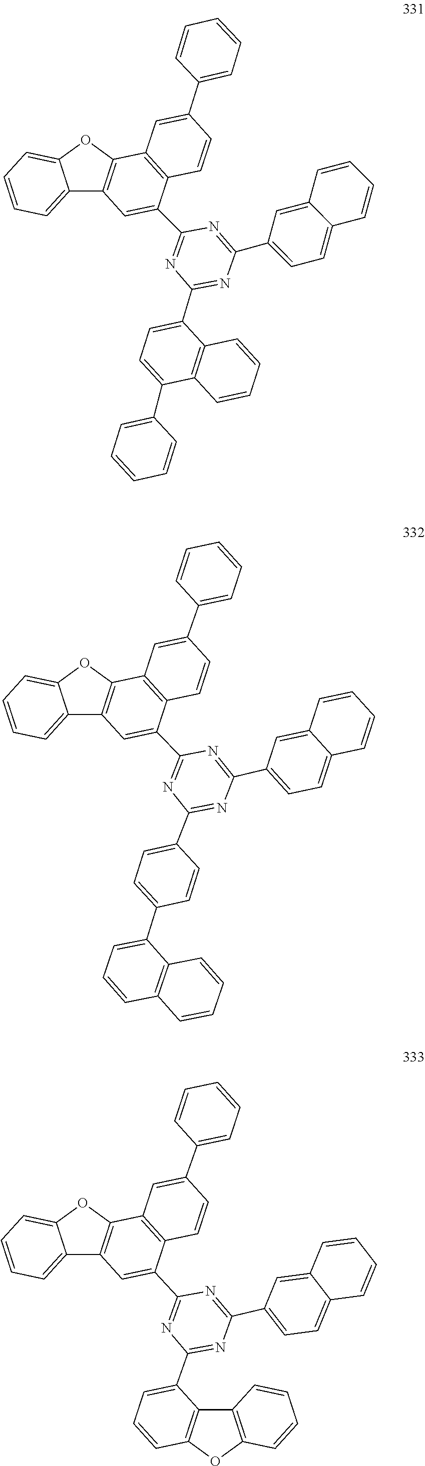 [SMILES: c1ccc(-c2ccc3c(-c4nc(-c5ccc6ccccc6c5)nc(-c5ccc(-c6ccccc6)c6ccccc56)n4)cc4c5ccccc5oc4c3c2)cc1 ; c1ccc(-c2ccc3c(-c4nc(-c5ccc(-c6cccc7ccccc67)cc5)nc(-c5ccc6ccccc6c5)n4)cc4c5ccccc5oc4c3c2)cc1 ; c1ccc(-c2ccc3c(-c4nc(-c5ccc6ccccc6c5)nc(-c5cccc6oc7ccccc7c56)n4)cc4c5ccccc5oc4c3c2)cc1]

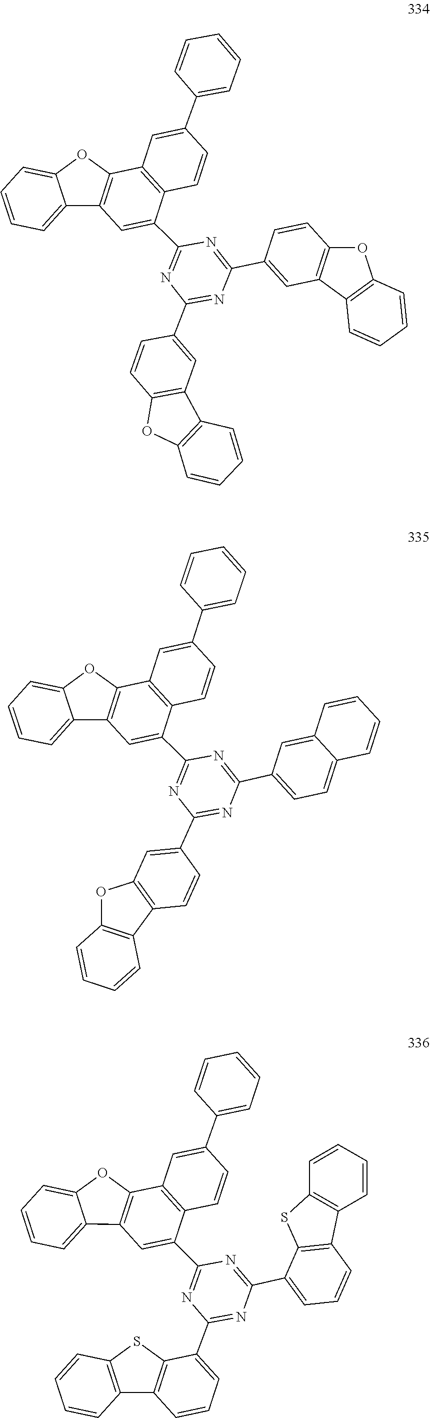 [SMILES: c1ccc(-c2ccc3c(-c4nc(-c5ccc6oc7ccccc7c6c5)nc(-c5ccc6oc7ccccc7c6c5)n4)cc4c5ccccc5oc4c3c2)cc1 ; c1ccc(-c2ccc3c(-c4nc(-c5ccc6ccccc6c5)nc(-c5ccc6c(c5)oc5ccccc56)n4)cc4c5ccccc5oc4c3c2)cc1 ; c1ccc(-c2ccc3c(-c4nc(-c5cccc6c5sc5ccccc56)nc(-c5cccc6c5sc5ccccc56)n4)cc4c5ccccc5oc4c3c2)cc1]

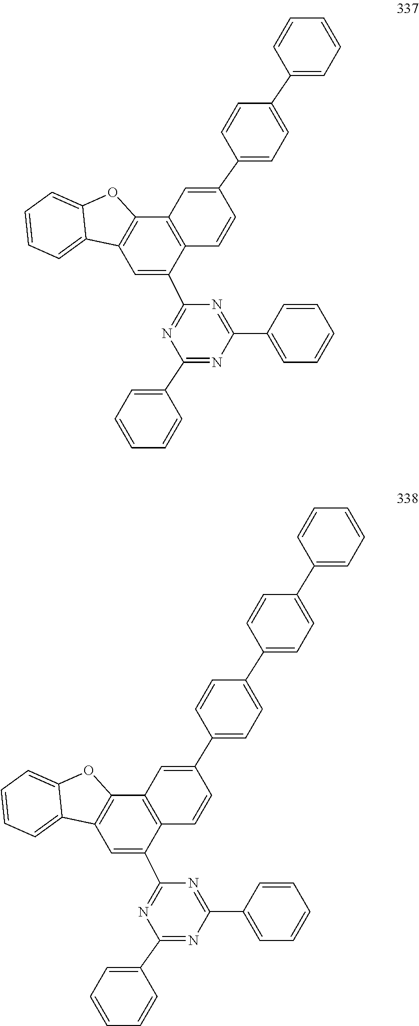 [SMILES: c1ccc(-c2ccc(-c3ccc4c(-c5nc(-c6ccccc6)nc(-c6ccccc6)n5)cc5c6ccccc6oc5c4c3)cc2)cc1 ; c1ccc(-c2ccc(-c3ccc(-c4ccc5c(-c6nc(-c7ccccc7)nc(-c7ccccc7)n6)cc6c7ccccc7oc6c5c4)cc3)cc2)cc1]

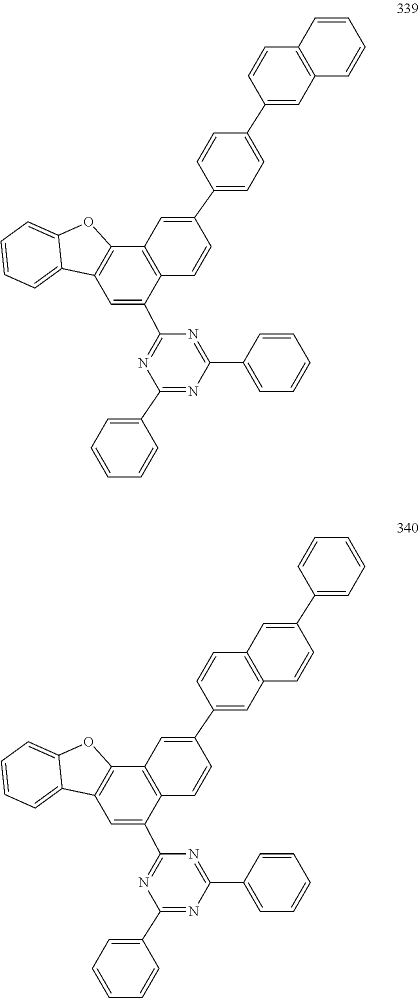 [SMILES: c1ccc(-c2nc(-c3ccccc3)nc(-c3cc4c5ccccc5oc4c4cc(-c5ccc(-c6ccc7ccccc7c6)cc5)ccc34)n2)cc1 ; c1ccc(-c2ccc3cc(-c4ccc5c(-c6nc(-c7ccccc7)nc(-c7ccccc7)n6)cc6c7ccccc7oc6c5c4)ccc3c2)cc1]

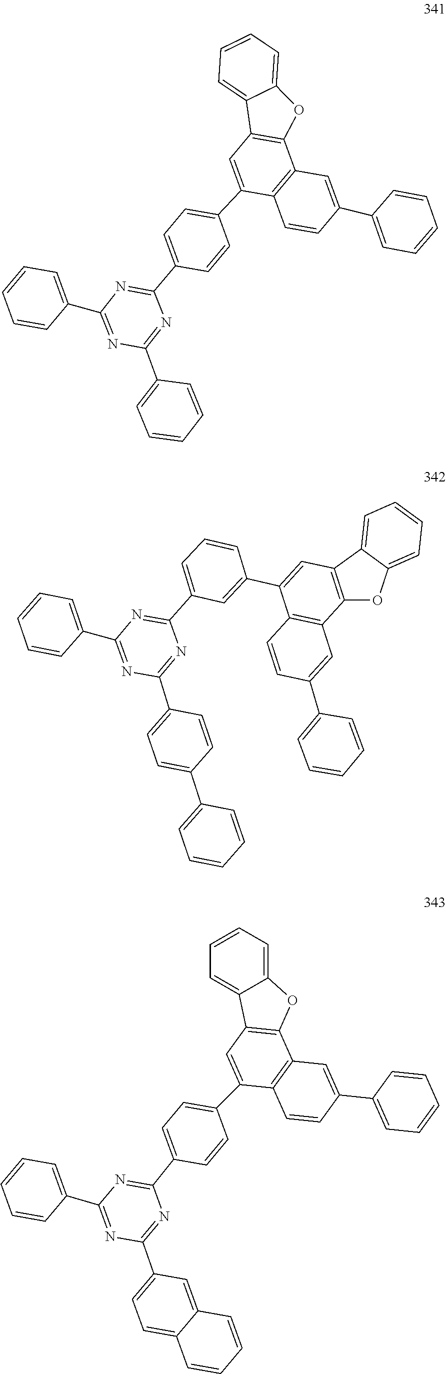 [SMILES: c1ccc(-c2ccc3c(-c4ccc(-c5nc(-c6ccccc6)nc(-c6ccccc6)n5)cc4)cc4c5ccccc5oc4c3c2)cc1 ; c1ccc(-c2ccc(-c3nc(-c4ccccc4)nc(-c4cccc(-c5cc6c7ccccc7oc6c6cc(-c7ccccc7)ccc56)c4)n3)cc2)cc1 ; c1ccc(-c2ccc3c(-c4ccc(-c5nc(-c6ccccc6)nc(-c6ccc7ccccc7c6)n5)cc4)cc4c5ccccc5oc4c3c2)cc1]

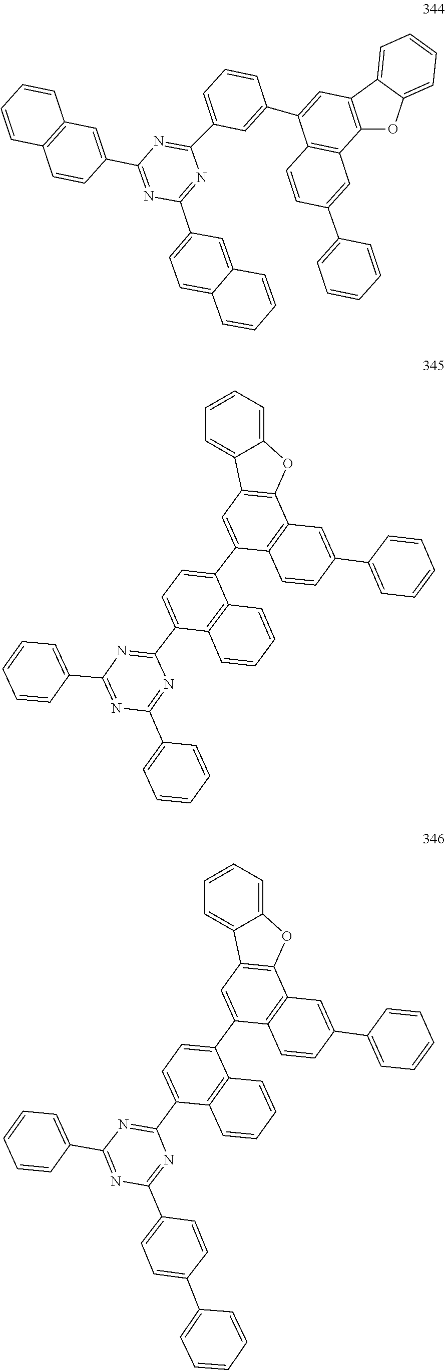 [SMILES: c1ccc(-c2ccc3c(-c4cccc(-c5nc(-c6ccc7ccccc7c6)nc(-c6ccc7ccccc7c6)n5)c4)cc4c5ccccc5oc4c3c2)cc1 ; c1ccc(-c2ccc3c(-c4ccc(-c5nc(-c6ccccc6)nc(-c6ccccc6)n5)c5ccccc45)cc4c5ccccc5oc4c3c2)cc1 ; c1ccc(-c2ccc(-c3nc(-c4ccccc4)nc(-c4ccc(-c5cc6c7ccccc7oc6c6cc(-c7ccccc7)ccc56)c5ccccc45)n3)cc2)cc1]

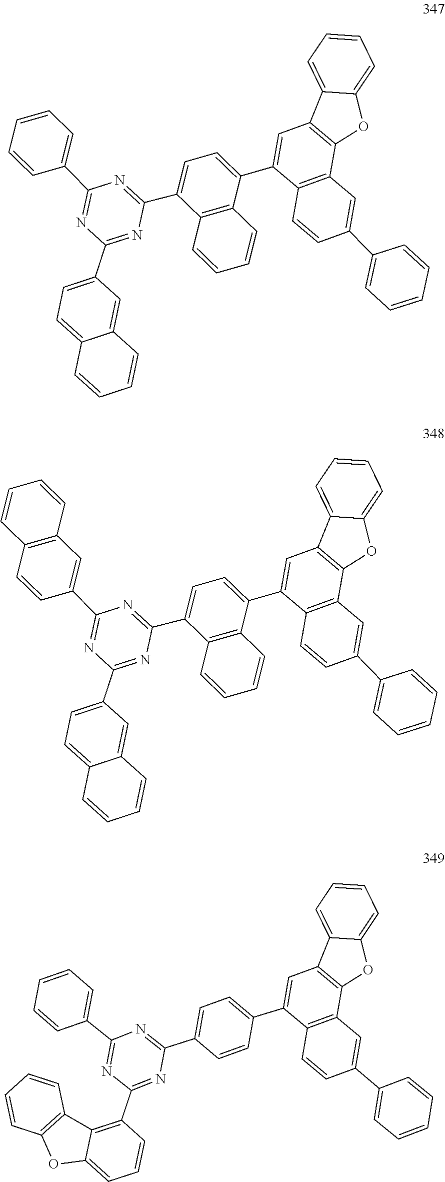 [SMILES: c1ccc(-c2ccc3c(-c4ccc(-c5nc(-c6ccccc6)nc(-c6ccc7ccccc7c6)n5)c5ccccc45)cc4c5ccccc5oc4c3c2)cc1 ; c1ccc(-c2ccc3c(-c4ccc(-c5nc(-c6ccc7ccccc7c6)nc(-c6ccc7ccccc7c6)n5)c5ccccc45)cc4c5ccccc5oc4c3c2)cc1 ; c1ccc(-c2ccc3c(-c4ccc(-c5nc(-c6ccccc6)nc(-c6cccc7oc8ccccc8c67)n5)cc4)cc4c5ccccc5oc4c3c2)cc1]

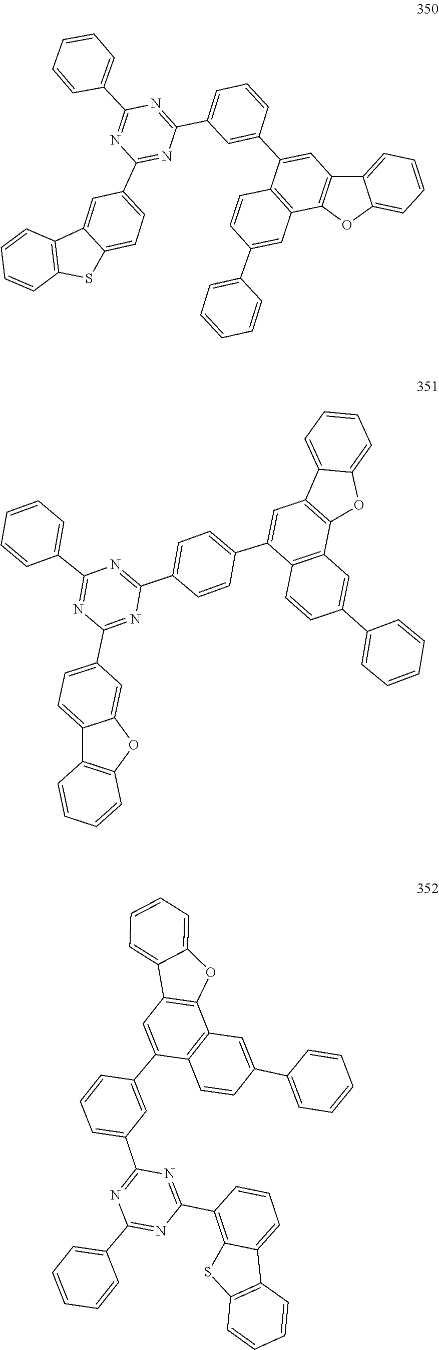 [SMILES: c1ccc(-c2ccc3c(-c4cccc(-c5nc(-c6ccccc6)nc(-c6ccc7sc8ccccc8c7c6)n5)c4)cc4c5ccccc5oc4c3c2)cc1 ; c1ccc(-c2ccc3c(-c4ccc(-c5nc(-c6ccccc6)nc(-c6ccc7c(c6)oc6ccccc67)n5)cc4)cc4c5ccccc5oc4c3c2)cc1 ; c1ccc(-c2ccc3c(-c4cccc(-c5nc(-c6ccccc6)nc(-c6cccc7c6sc6ccccc67)n5)c4)cc4c5ccccc5oc4c3c2)cc1]

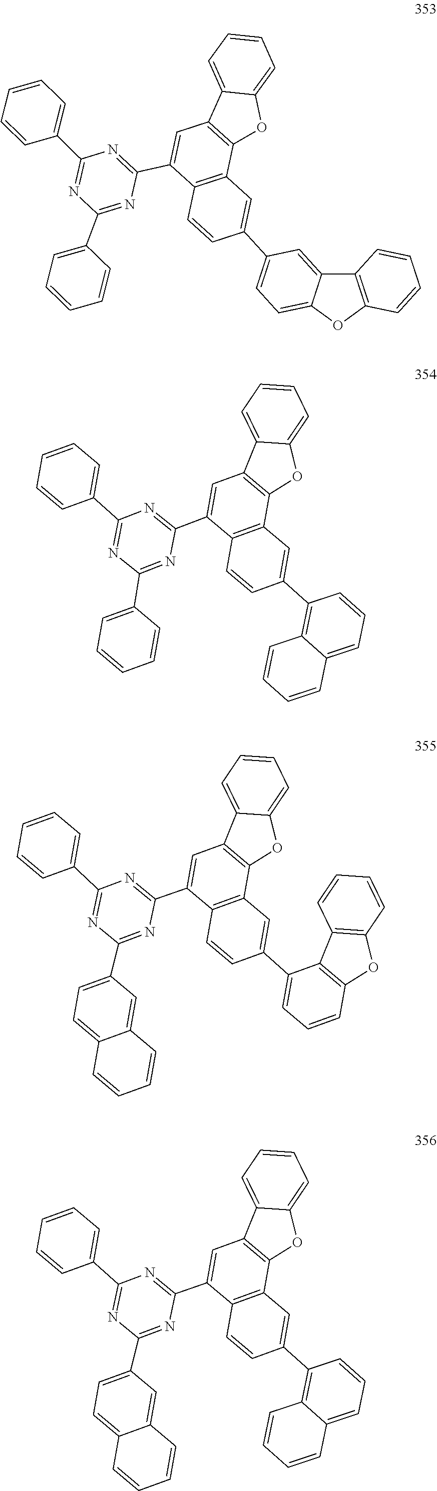 [SMILES: c1ccc(-c2nc(-c3ccccc3)nc(-c3cc4c5ccccc5oc4c4cc(-c5ccc6oc7ccccc7c6c5)ccc34)n2)cc1 ; c1ccc(-c2nc(-c3ccccc3)nc(-c3cc4c5ccccc5oc4c4cc(-c5cccc6ccccc56)ccc34)n2)cc1 ; c1ccc(-c2nc(-c3ccc4ccccc4c3)nc(-c3cc4c5ccccc5oc4c4cc(-c5cccc6oc7ccccc7c56)ccc34)n2)cc1 ; c1ccc(-c2nc(-c3ccc4ccccc4c3)nc(-c3cc4c5ccccc5oc4c4cc(-c5cccc6ccccc56)ccc34)n2)cc1]

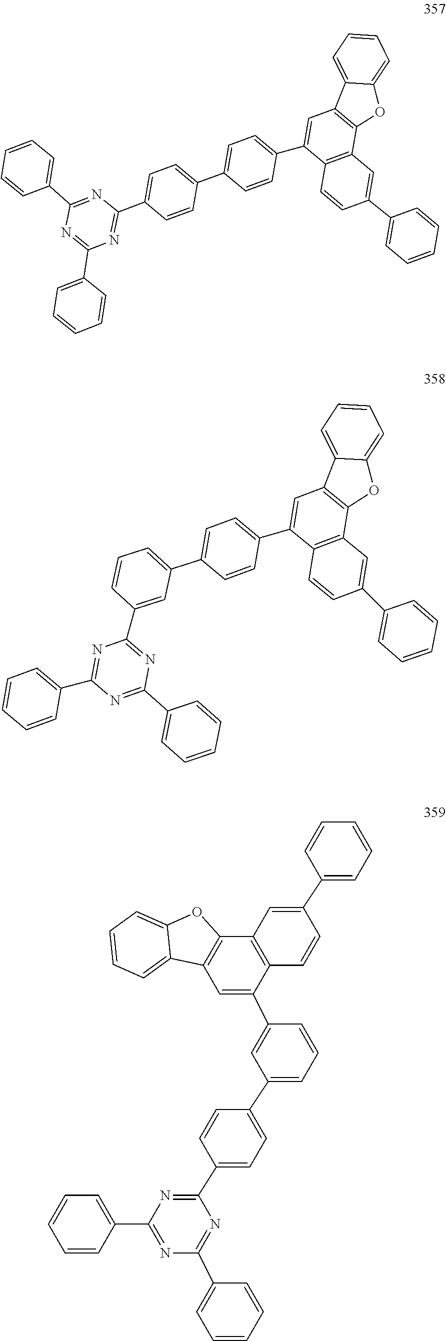 [SMILES: c1ccc(-c2ccc3c(-c4ccc(-c5ccc(-c6nc(-c7ccccc7)nc(-c7ccccc7)n6)cc5)cc4)cc4c5ccccc5oc4c3c2)cc1 ; c1ccc(-c2ccc3c(-c4ccc(-c5cccc(-c6nc(-c7ccccc7)nc(-c7ccccc7)n6)c5)cc4)cc4c5ccccc5oc4c3c2)cc1 ; c1ccc(-c2ccc3c(-c4cccc(-c5ccc(-c6nc(-c7ccccc7)nc(-c7ccccc7)n6)cc5)c4)cc4c5ccccc5oc4c3c2)cc1]

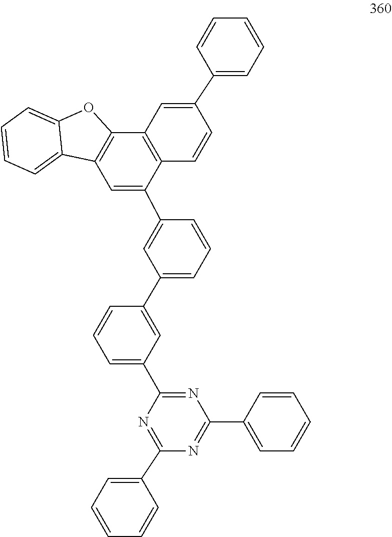 [SMILES: c1ccc(-c2ccc3c(-c4cccc(-c5cccc(-c6nc(-c7ccccc7)nc(-c7ccccc7)n6)c5)c4)cc4c5ccccc5oc4c3c2)cc1]

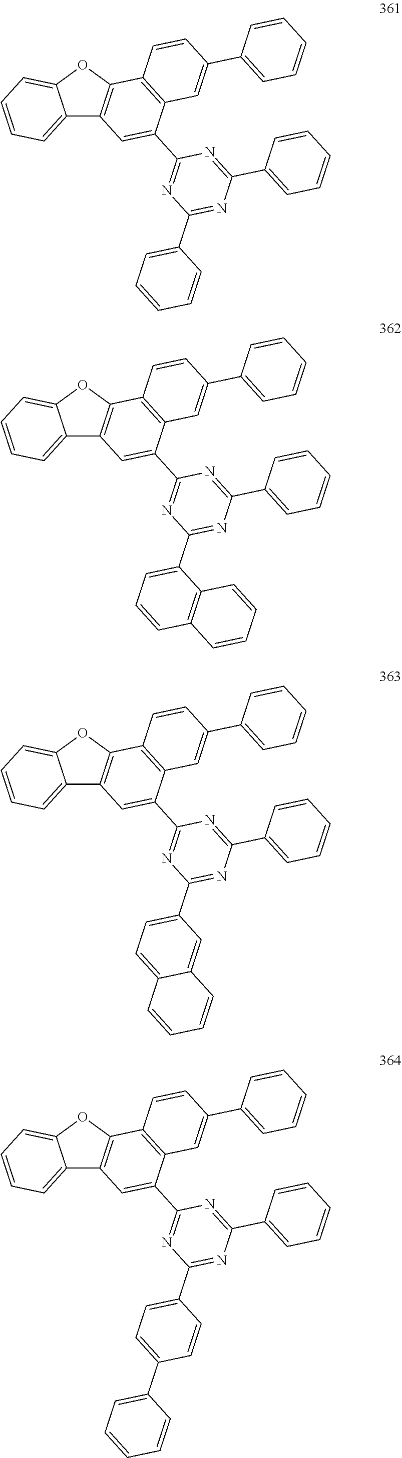 [SMILES: c1ccc(-c2ccc3c(c2)c(-c2nc(-c4ccccc4)nc(-c4ccccc4)n2)cc2c4ccccc4oc32)cc1 ; c1ccc(-c2ccc3c(c2)c(-c2nc(-c4ccccc4)nc(-c4cccc5ccccc45)n2)cc2c4ccccc4oc32)cc1 ; c1ccc(-c2ccc3c(c2)c(-c2nc(-c4ccccc4)nc(-c4ccc5ccccc5c4)n2)cc2c4ccccc4oc32)cc1 ; c1ccc(-c2ccc(-c3nc(-c4ccccc4)nc(-c4cc5c6ccccc6oc5c5ccc(-c6ccccc6)cc45)n3)cc2)cc1]

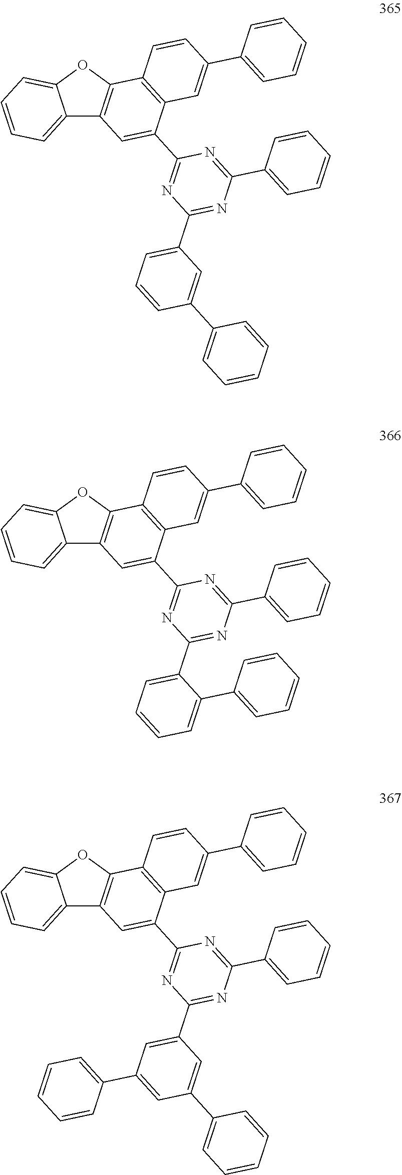 [SMILES: c1ccc(-c2cccc(-c3nc(-c4ccccc4)nc(-c4cc5c6ccccc6oc5c5ccc(-c6ccccc6)cc45)n3)c2)cc1 ; c1ccc(-c2ccc3c(c2)c(-c2nc(-c4ccccc4)nc(-c4ccccc4-c4ccccc4)n2)cc2c4ccccc4oc32)cc1 ; c1ccc(-c2cc(-c3ccccc3)cc(-c3nc(-c4ccccc4)nc(-c4cc5c6ccccc6oc5c5ccc(-c6ccccc6)cc45)n3)c2)cc1]

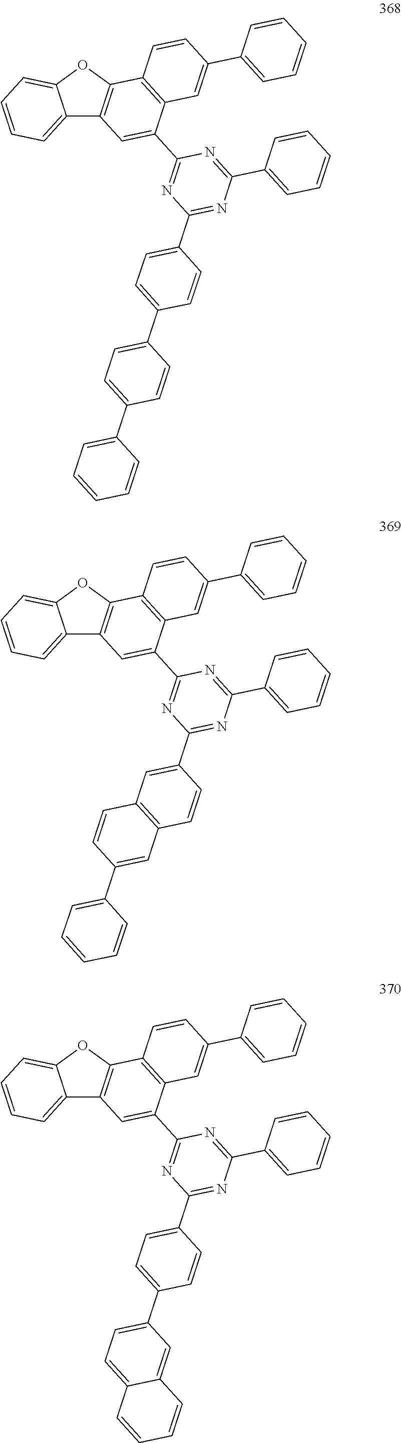 [SMILES: c1ccc(-c2ccc(-c3ccc(-c4nc(-c5ccccc5)nc(-c5cc6c7ccccc7oc6c6ccc(-c7ccccc7)cc56)n4)cc3)cc2)cc1 ; c1ccc(-c2ccc3cc(-c4nc(-c5ccccc5)nc(-c5cc6c7ccccc7oc6c6ccc(-c7ccccc7)cc56)n4)ccc3c2)cc1 ; c1ccc(-c2ccc3c(c2)c(-c2nc(-c4ccccc4)nc(-c4ccc(-c5ccc6ccccc6c5)cc4)n2)cc2c4ccccc4oc32)cc1]

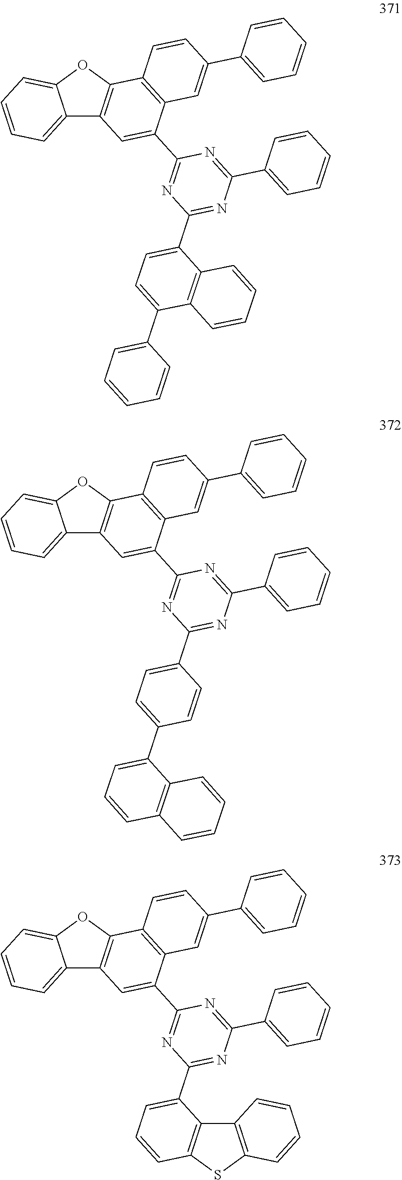 [SMILES: c1ccc(-c2ccc3c(c2)c(-c2nc(-c4ccccc4)nc(-c4ccc(-c5ccccc5)c5ccccc45)n2)cc2c4ccccc4oc32)cc1 ; c1ccc(-c2ccc3c(c2)c(-c2nc(-c4ccccc4)nc(-c4ccc(-c5cccc6ccccc56)cc4)n2)cc2c4ccccc4oc32)cc1 ; c1ccc(-c2ccc3c(c2)c(-c2nc(-c4ccccc4)nc(-c4cccc5sc6ccccc6c45)n2)cc2c4ccccc4oc32)cc1]

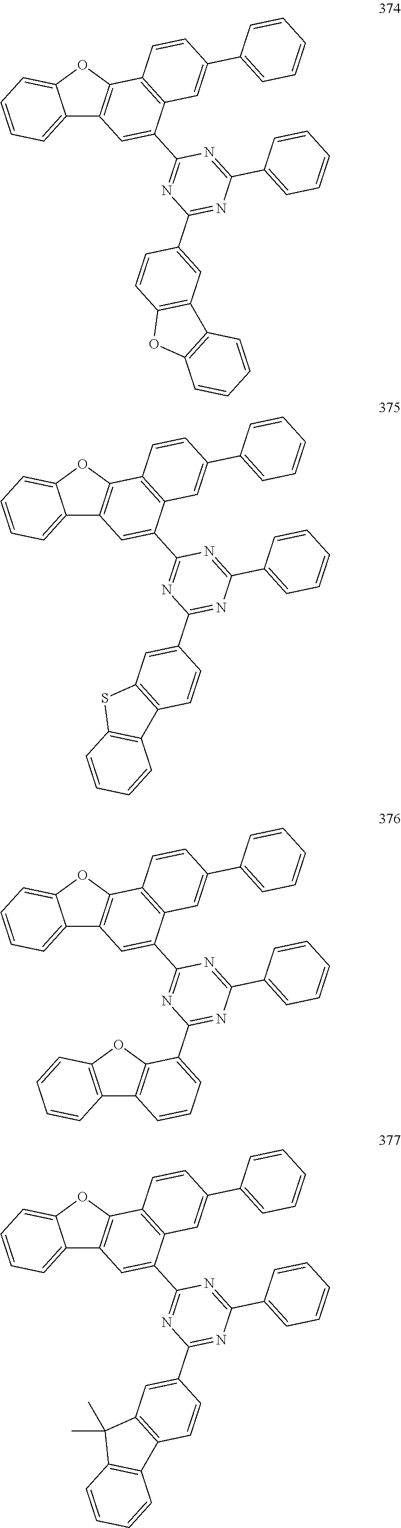

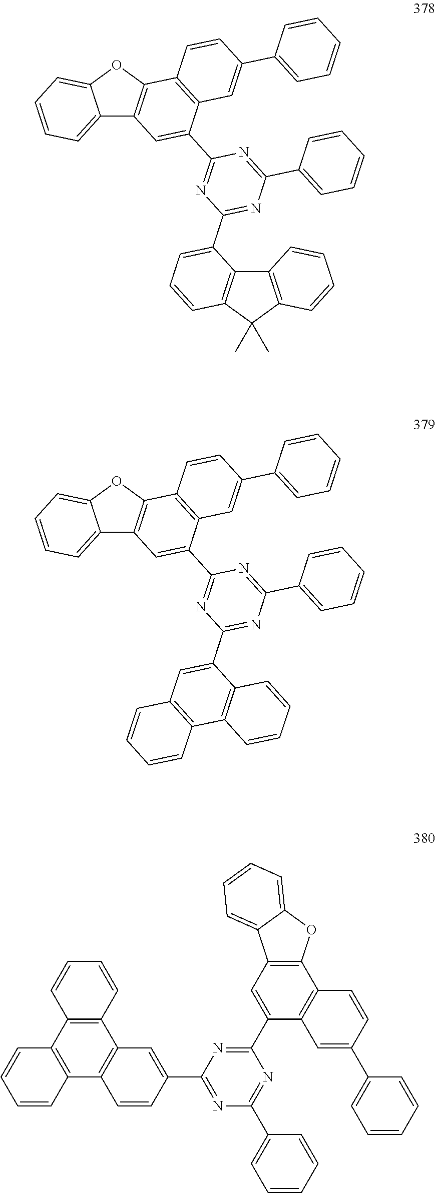 [SMILES: CC1(C)c2ccccc2-c2c(-c3nc(-c4ccccc4)nc(-c4cc5c6ccccc6oc5c5ccc(-c6ccccc6)cc45)n3)cccc21 ; c1ccc(-c2ccc3c(c2)c(-c2nc(-c4ccccc4)nc(-c4cc5ccccc5c5ccccc45)n2)cc2c4ccccc4oc32)cc1 ; c1ccc(-c2ccc3c(c2)c(-c2nc(-c4ccccc4)nc(-c4ccc5c6ccccc6c6ccccc6c5c4)n2)cc2c4ccccc4oc32)cc1]

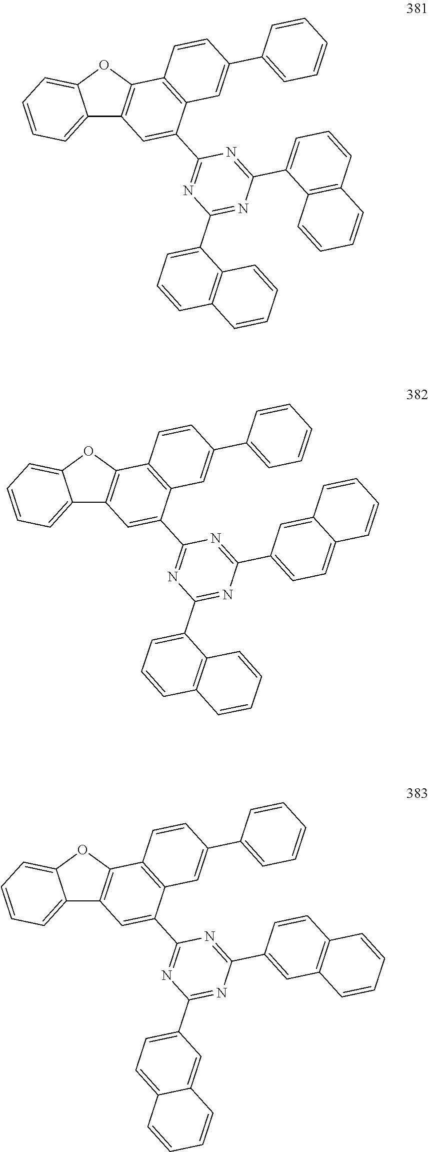 [SMILES: c1ccc(-c2ccc3c(c2)c(-c2nc(-c4cccc5ccccc45)nc(-c4cccc5ccccc45)n2)cc2c4ccccc4oc32)cc1 ; c1ccc(-c2ccc3c(c2)c(-c2nc(-c4ccc5ccccc5c4)nc(-c4cccc5ccccc45)n2)cc2c4ccccc4oc32)cc1 ; c1ccc(-c2ccc3c(c2)c(-c2nc(-c4ccc5ccccc5c4)nc(-c4ccc5ccccc5c4)n2)cc2c4ccccc4oc32)cc1]

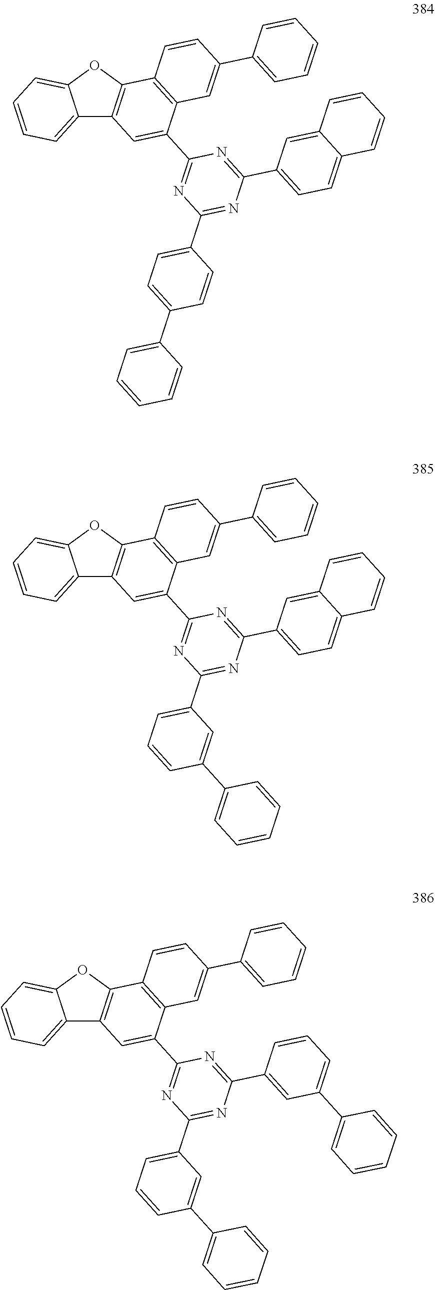 [SMILES: c1ccc(-c2ccc(-c3nc(-c4ccc5ccccc5c4)nc(-c4cc5c6ccccc6oc5c5ccc(-c6ccccc6)cc45)n3)cc2)cc1 ; c1ccc(-c2cccc(-c3nc(-c4ccc5ccccc5c4)nc(-c4cc5c6ccccc6oc5c5ccc(-c6ccccc6)cc45)n3)c2)cc1 ; c1ccc(-c2cccc(-c3nc(-c4cccc(-c5ccccc5)c4)nc(-c4cc5c6ccccc6oc5c5ccc(-c6ccccc6)cc45)n3)c2)cc1]

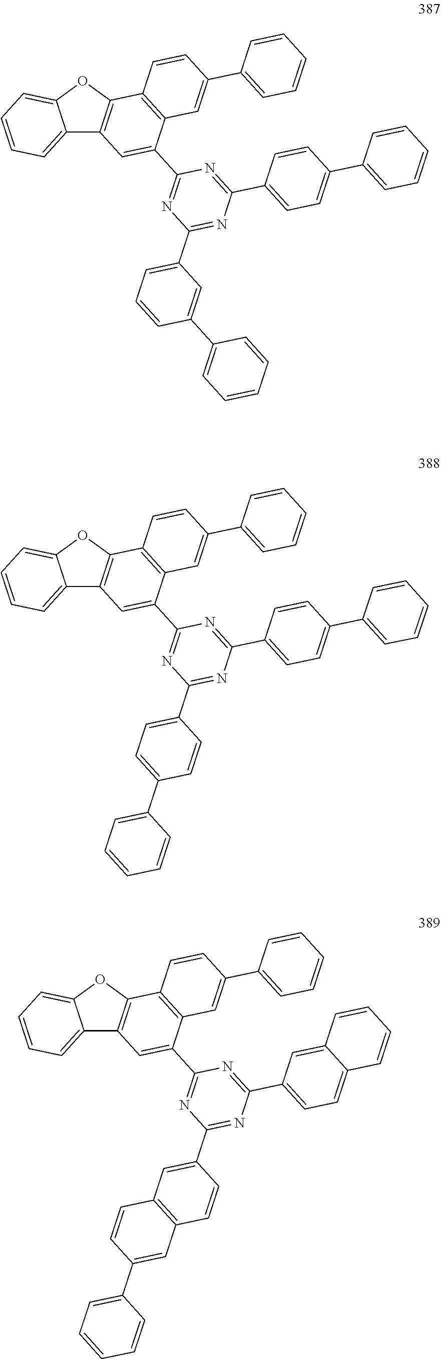 [SMILES: c1ccc(-c2ccc(-c3nc(-c4cccc(-c5ccccc5)c4)nc(-c4cc5c6ccccc6oc5c5ccc(-c6ccccc6)cc45)n3)cc2)cc1 ; c1ccc(-c2ccc(-c3nc(-c4ccc(-c5ccccc5)cc4)nc(-c4cc5c6ccccc6oc5c5ccc(-c6ccccc6)cc45)n3)cc2)cc1 ; c1ccc(-c2ccc3cc(-c4nc(-c5ccc6ccccc6c5)nc(-c5cc6c7ccccc7oc6c6ccc(-c7ccccc7)cc56)n4)ccc3c2)cc1]

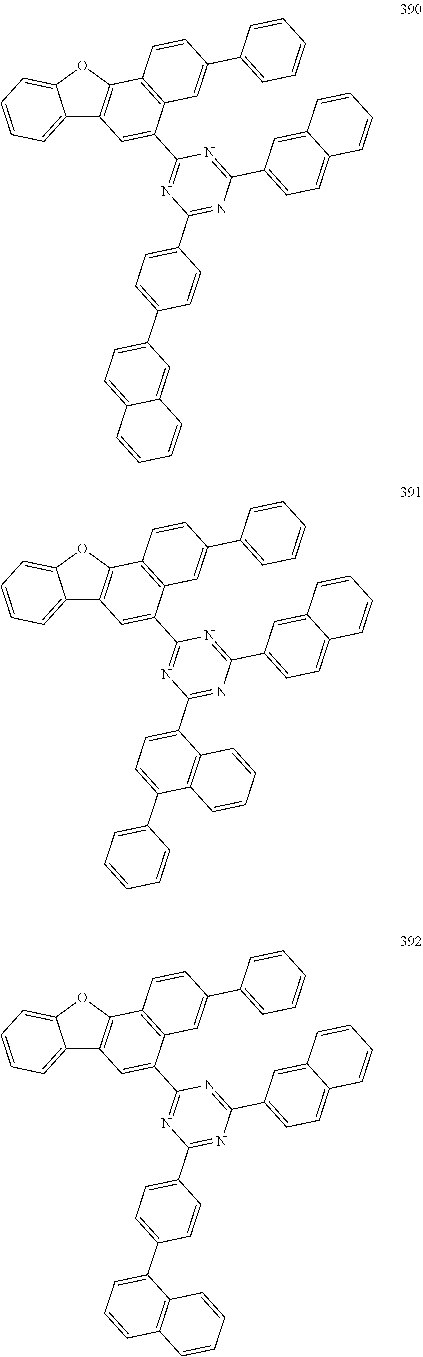 [SMILES: c1ccc(-c2ccc3c(c2)c(-c2nc(-c4ccc(-c5ccc6ccccc6c5)cc4)nc(-c4ccc5ccccc5c4)n2)cc2c4ccccc4oc32)cc1 ; c1ccc(-c2ccc3c(c2)c(-c2nc(-c4ccc5ccccc5c4)nc(-c4ccc(-c5ccccc5)c5ccccc45)n2)cc2c4ccccc4oc32)cc1 ; c1ccc(-c2ccc3c(c2)c(-c2nc(-c4ccc(-c5cccc6ccccc56)cc4)nc(-c4ccc5ccccc5c4)n2)cc2c4ccccc4oc32)cc1]

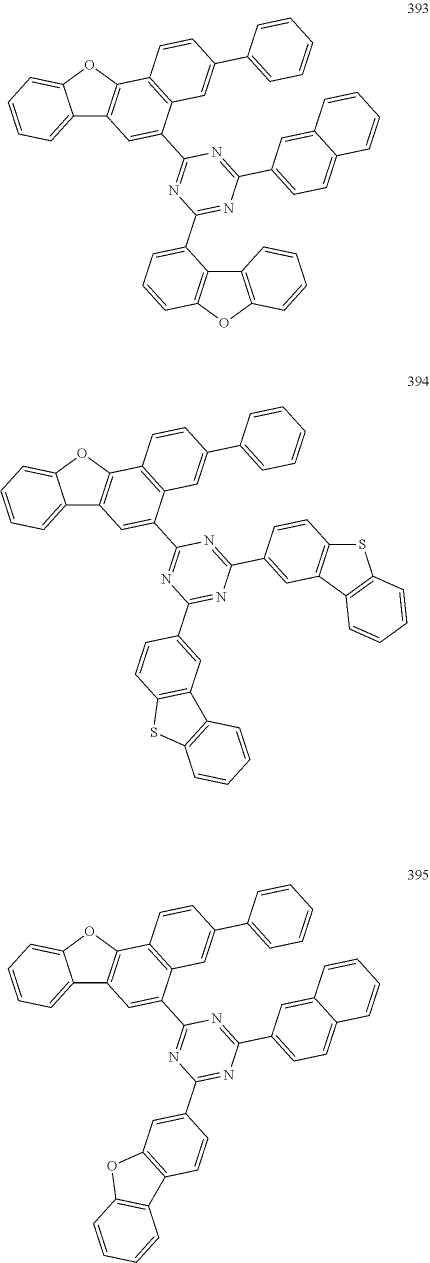 [SMILES: c1ccc(-c2ccc3c(c2)c(-c2nc(-c4ccc5ccccc5c4)nc(-c4cccc5oc6ccccc6c45)n2)cc2c4ccccc4oc32)cc1 ; c1ccc(-c2ccc3c(c2)c(-c2nc(-c4ccc5sc6ccccc6c5c4)nc(-c4ccc5sc6ccccc6c5c4)n2)cc2c4ccccc4oc32)cc1 ; c1ccc(-c2ccc3c(c2)c(-c2nc(-c4ccc5ccccc5c4)nc(-c4ccc5c(c4)oc4ccccc45)n2)cc2c4ccccc4oc32)cc1]

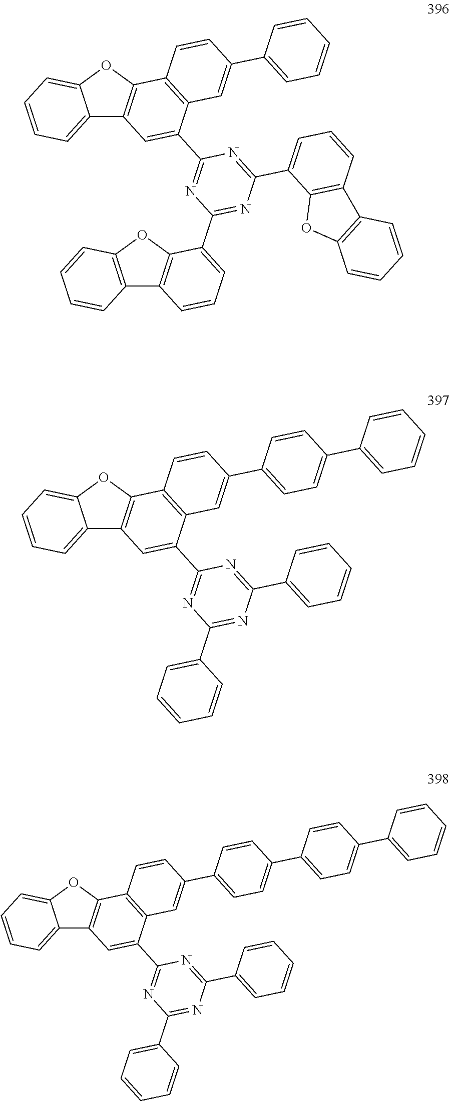 [SMILES: c1ccc(-c2ccc3c(c2)c(-c2nc(-c4cccc5c4oc4ccccc45)nc(-c4cccc5c4oc4ccccc45)n2)cc2c4ccccc4oc32)cc1 ; c1ccc(-c2ccc(-c3ccc4c(c3)c(-c3nc(-c5ccccc5)nc(-c5ccccc5)n3)cc3c5ccccc5oc43)cc2)cc1 ; c1ccc(-c2ccc(-c3ccc(-c4ccc5c(c4)c(-c4nc(-c6ccccc6)nc(-c6ccccc6)n4)cc4c6ccccc6oc54)cc3)cc2)cc1]

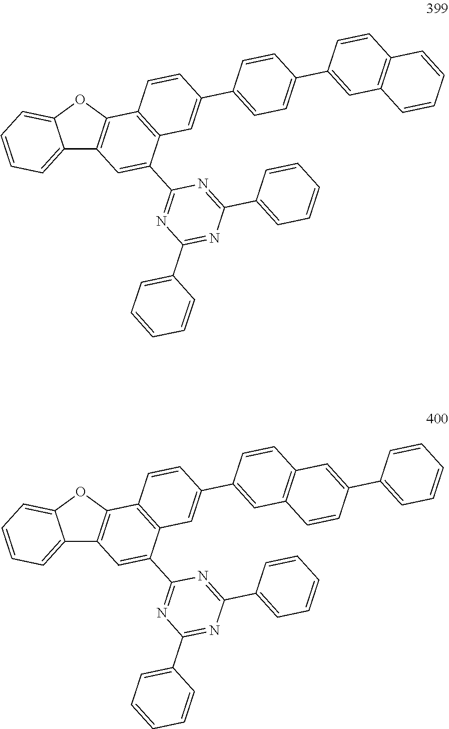 [SMILES: c1ccc(-c2nc(-c3ccccc3)nc(-c3cc4c5ccccc5oc4c4ccc(-c5ccc(-c6ccc7ccccc7c6)cc5)cc34)n2)cc1 ; c1ccc(-c2ccc3cc(-c4ccc5c(c4)c(-c4nc(-c6ccccc6)nc(-c6ccccc6)n4)cc4c6ccccc6oc54)ccc3c2)cc1]

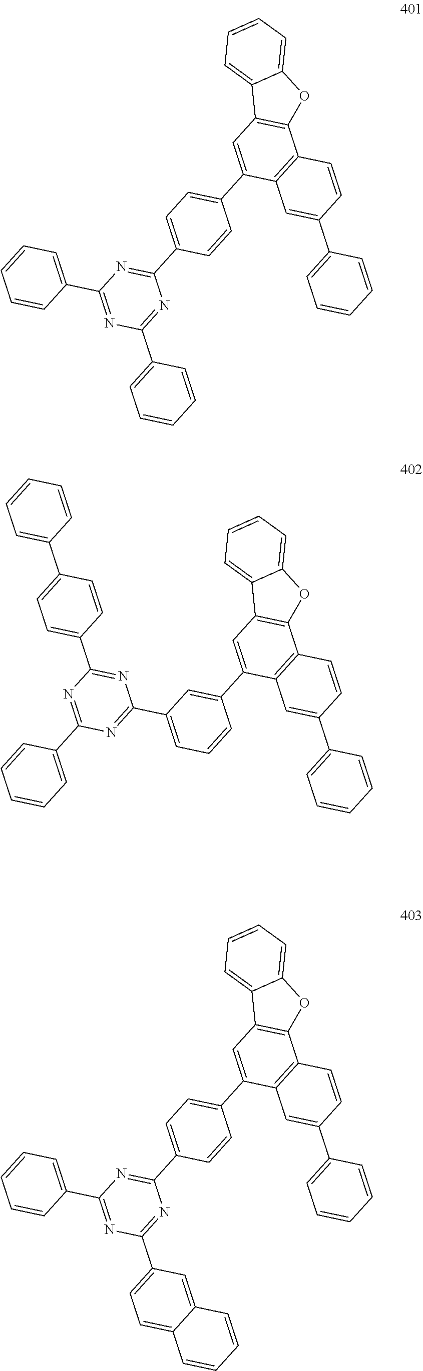 [SMILES: c1ccc(-c2ccc3c(c2)c(-c2ccc(-c4nc(-c5ccccc5)nc(-c5ccccc5)n4)cc2)cc2c4ccccc4oc32)cc1 ; c1ccc(-c2ccc(-c3nc(-c4ccccc4)nc(-c4cccc(-c5cc6c7ccccc7oc6c6ccc(-c7ccccc7)cc56)c4)n3)cc2)cc1 ; c1ccc(-c2ccc3c(c2)c(-c2ccc(-c4nc(-c5ccccc5)nc(-c5ccc6ccccc6c5)n4)cc2)cc2c4ccccc4oc32)cc1]

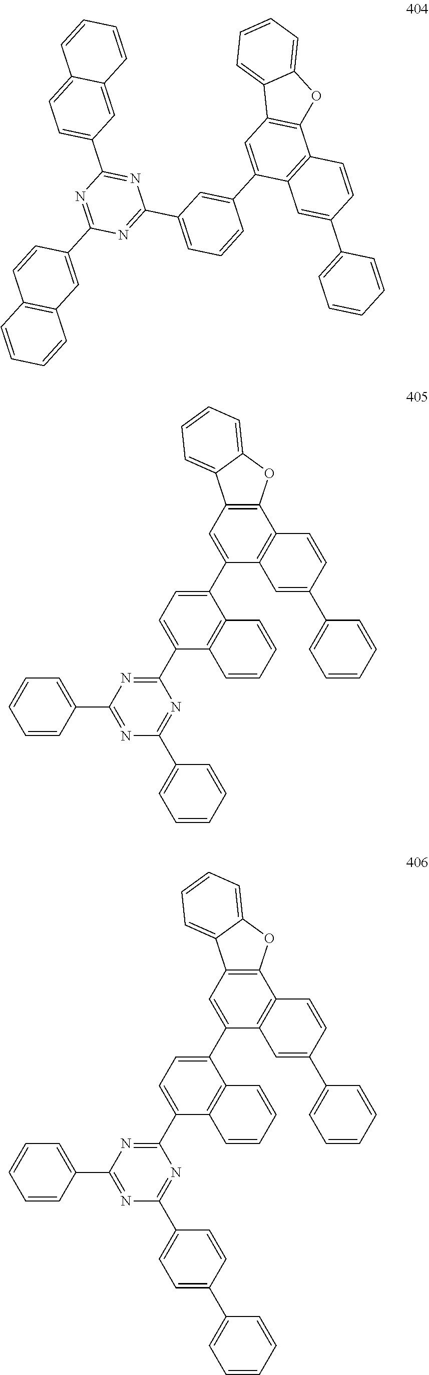 [SMILES: c1ccc(-c2ccc3c(c2)c(-c2cccc(-c4nc(-c5ccc6ccccc6c5)nc(-c5ccc6ccccc6c5)n4)c2)cc2c4ccccc4oc32)cc1 ; c1ccc(-c2ccc3c(c2)c(-c2ccc(-c4nc(-c5ccccc5)nc(-c5ccccc5)n4)c4ccccc24)cc2c4ccccc4oc32)cc1 ; c1ccc(-c2ccc(-c3nc(-c4ccccc4)nc(-c4ccc(-c5cc6c7ccccc7oc6c6ccc(-c7ccccc7)cc56)c5ccccc45)n3)cc2)cc1]

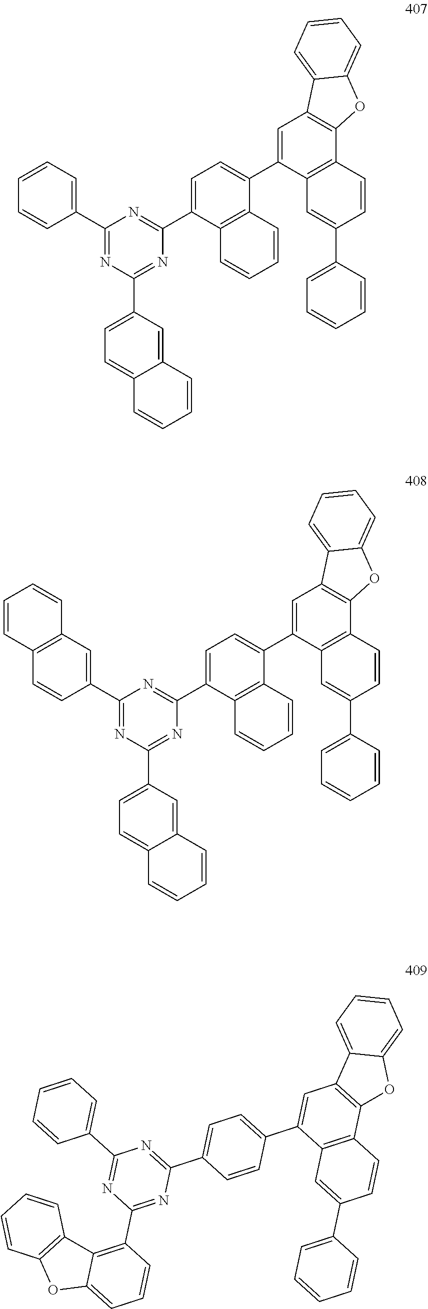 [SMILES: c1ccc(-c2ccc3c(c2)c(-c2ccc(-c4nc(-c5ccccc5)nc(-c5ccc6ccccc6c5)n4)c4ccccc24)cc2c4ccccc4oc32)cc1 ; c1ccc(-c2ccc3c(c2)c(-c2ccc(-c4nc(-c5ccc6ccccc6c5)nc(-c5ccc6ccccc6c5)n4)c4ccccc24)cc2c4ccccc4oc32)cc1 ; c1ccc(-c2ccc3c(c2)c(-c2ccc(-c4nc(-c5ccccc5)nc(-c5cccc6oc7ccccc7c56)n4)cc2)cc2c4ccccc4oc32)cc1]

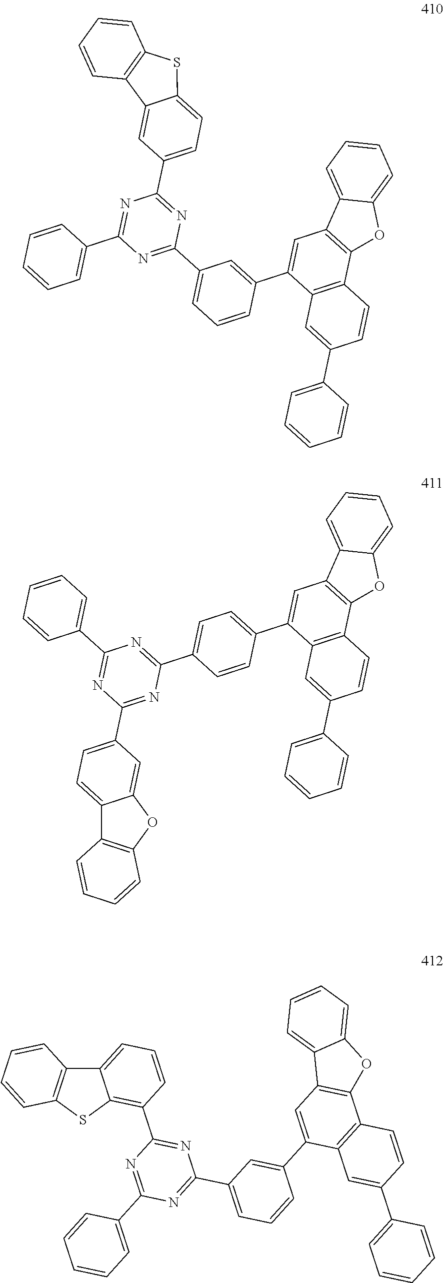 [SMILES: c1ccc(-c2ccc3c(c2)c(-c2cccc(-c4nc(-c5ccccc5)nc(-c5ccc6sc7ccccc7c6c5)n4)c2)cc2c4ccccc4oc32)cc1 ; c1ccc(-c2ccc3c(c2)c(-c2ccc(-c4nc(-c5ccccc5)nc(-c5ccc6c(c5)oc5ccccc56)n4)cc2)cc2c4ccccc4oc32)cc1 ; c1ccc(-c2ccc3c(c2)c(-c2cccc(-c4nc(-c5ccccc5)nc(-c5cccc6c5sc5ccccc56)n4)c2)cc2c4ccccc4oc32)cc1]

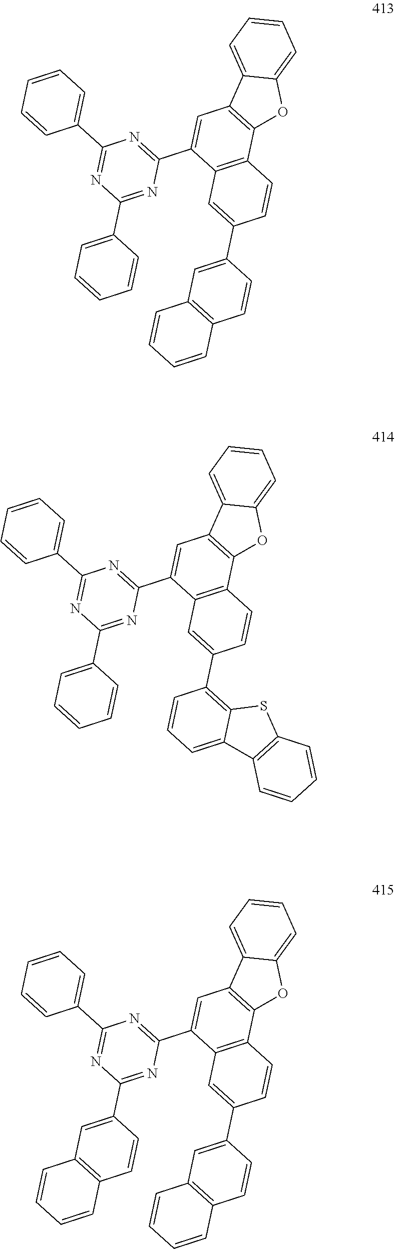 [SMILES: c1ccc(-c2nc(-c3ccccc3)nc(-c3cc4c5ccccc5oc4c4ccc(-c5ccc6ccccc6c5)cc34)n2)cc1 ; c1ccc(-c2nc(-c3ccccc3)nc(-c3cc4c5ccccc5oc4c4ccc(-c5cccc6c5sc5ccccc56)cc34)n2)cc1 ; c1ccc(-c2nc(-c3ccc4ccccc4c3)nc(-c3cc4c5ccccc5oc4c4ccc(-c5ccc6ccccc6c5)cc34)n2)cc1]

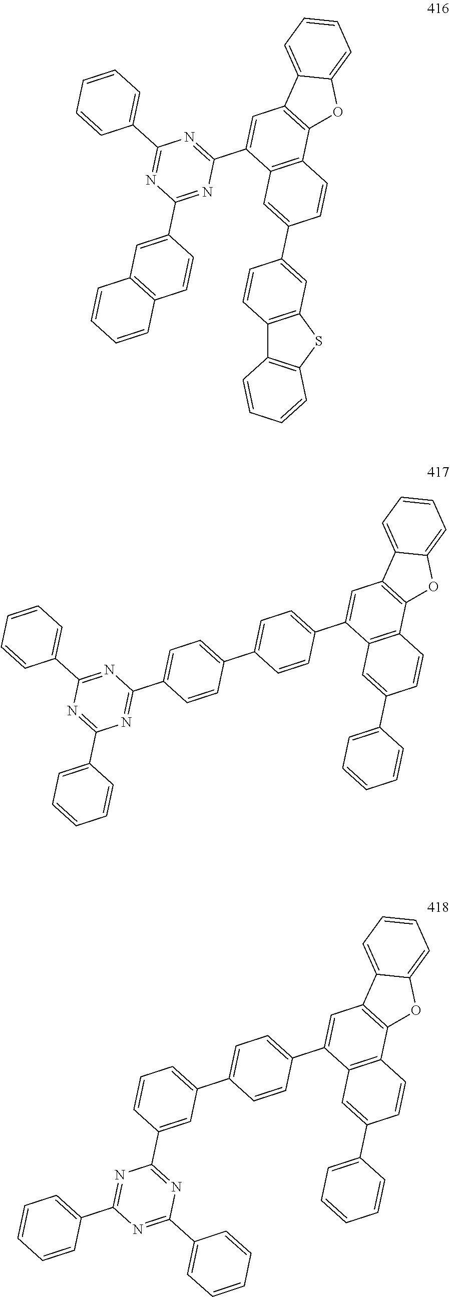 [SMILES: c1ccc(-c2nc(-c3ccc4ccccc4c3)nc(-c3cc4c5ccccc5oc4c4ccc(-c5ccc6c(c5)sc5ccccc56)cc34)n2)cc1 ; c1ccc(-c2ccc3c(c2)c(-c2ccc(-c4ccc(-c5nc(-c6ccccc6)nc(-c6ccccc6)n5)cc4)cc2)cc2c4ccccc4oc32)cc1 ; c1ccc(-c2ccc3c(c2)c(-c2ccc(-c4cccc(-c5nc(-c6ccccc6)nc(-c6ccccc6)n5)c4)cc2)cc2c4ccccc4oc32)cc1]

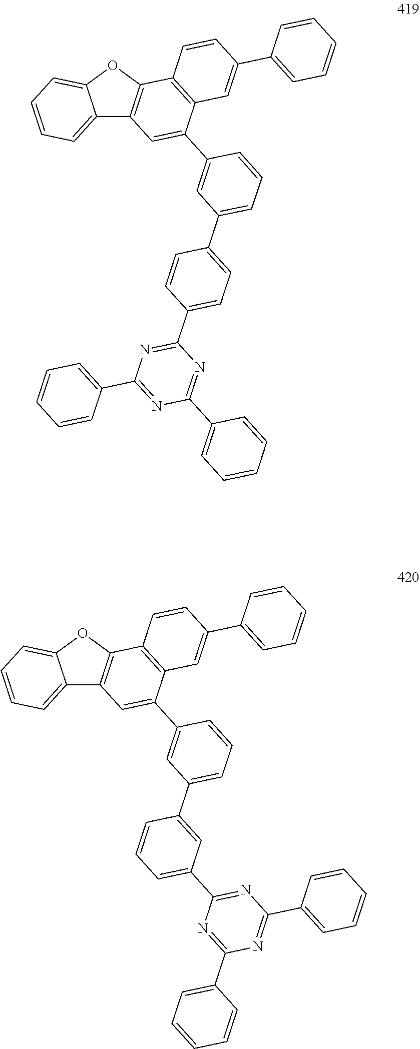 [SMILES: c1ccc(-c2ccc3c(c2)c(-c2cccc(-c4ccc(-c5nc(-c6ccccc6)nc(-c6ccccc6)n5)cc4)c2)cc2c4ccccc4oc32)cc1 ; c1ccc(-c2ccc3c(c2)c(-c2cccc(-c4cccc(-c5nc(-c6ccccc6)nc(-c6ccccc6)n5)c4)c2)cc2c4ccccc4oc32)cc1]

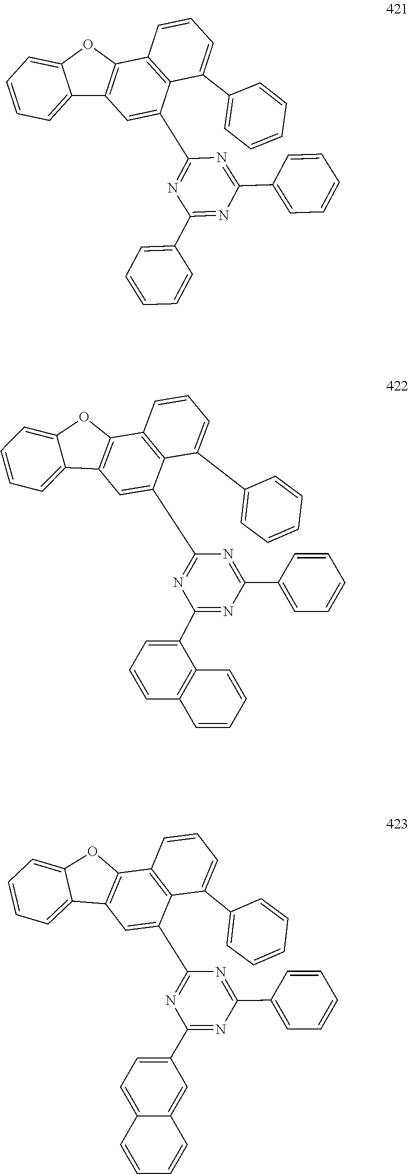 [SMILES: c1ccc(-c2nc(-c3ccccc3)nc(-c3cc4c5ccccc5oc4c4cccc(-c5ccccc5)c34)n2)cc1 ; c1ccc(-c2nc(-c3cccc4ccccc34)nc(-c3cc4c5ccccc5oc4c4cccc(-c5ccccc5)c34)n2)cc1 ; c1ccc(-c2nc(-c3ccc4ccccc4c3)nc(-c3cc4c5ccccc5oc4c4cccc(-c5ccccc5)c34)n2)cc1]

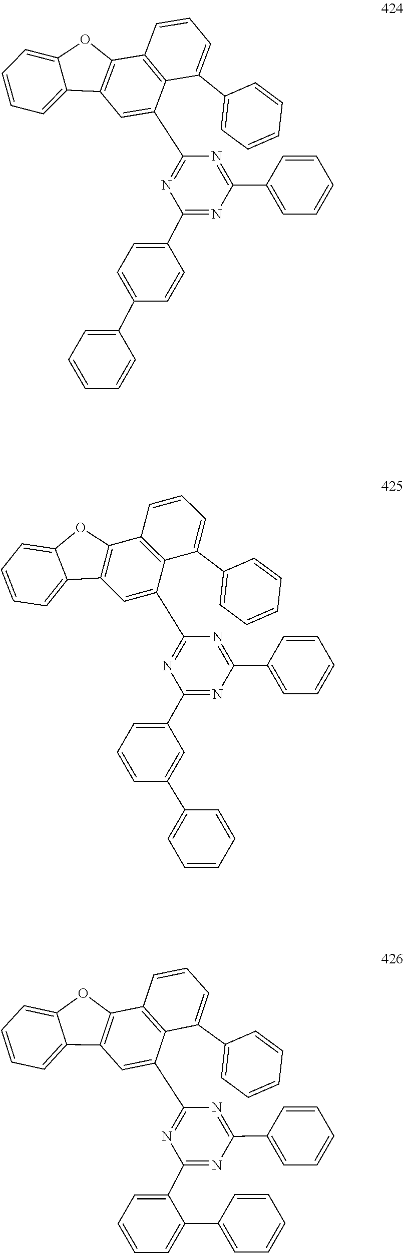 [SMILES: c1ccc(-c2ccc(-c3nc(-c4ccccc4)nc(-c4cc5c6ccccc6oc5c5cccc(-c6ccccc6)c45)n3)cc2)cc1 ; c1ccc(-c2cccc(-c3nc(-c4ccccc4)nc(-c4cc5c6ccccc6oc5c5cccc(-c6ccccc6)c45)n3)c2)cc1 ; c1ccc(-c2nc(-c3ccccc3-c3ccccc3)nc(-c3cc4c5ccccc5oc4c4cccc(-c5ccccc5)c34)n2)cc1]

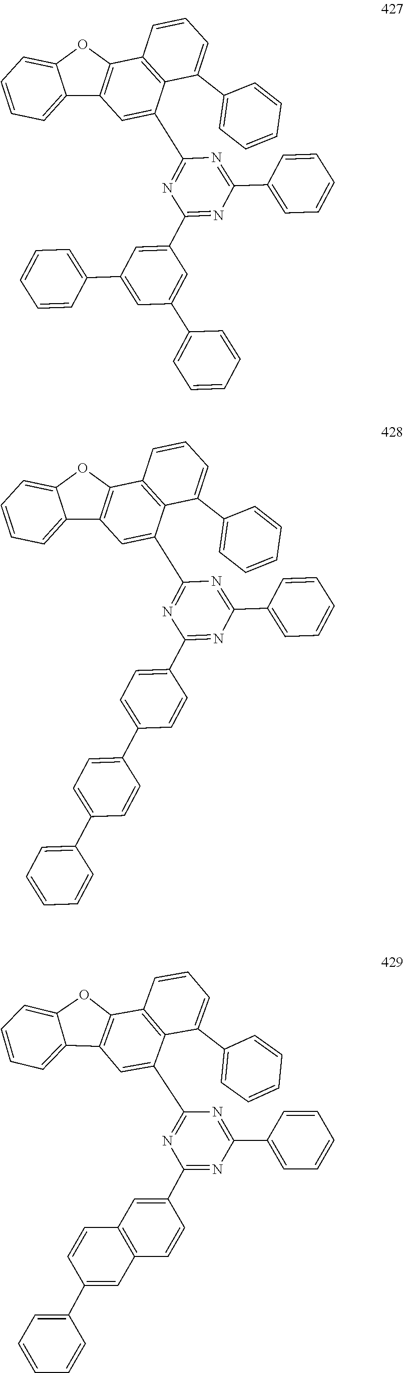 [SMILES: c1ccc(-c2cc(-c3ccccc3)cc(-c3nc(-c4ccccc4)nc(-c4cc5c6ccccc6oc5c5cccc(-c6ccccc6)c45)n3)c2)cc1 ; c1ccc(-c2ccc(-c3ccc(-c4nc(-c5ccccc5)nc(-c5cc6c7ccccc7oc6c6cccc(-c7ccccc7)c56)n4)cc3)cc2)cc1 ; c1ccc(-c2ccc3cc(-c4nc(-c5ccccc5)nc(-c5cc6c7ccccc7oc6c6cccc(-c7ccccc7)c56)n4)ccc3c2)cc1]

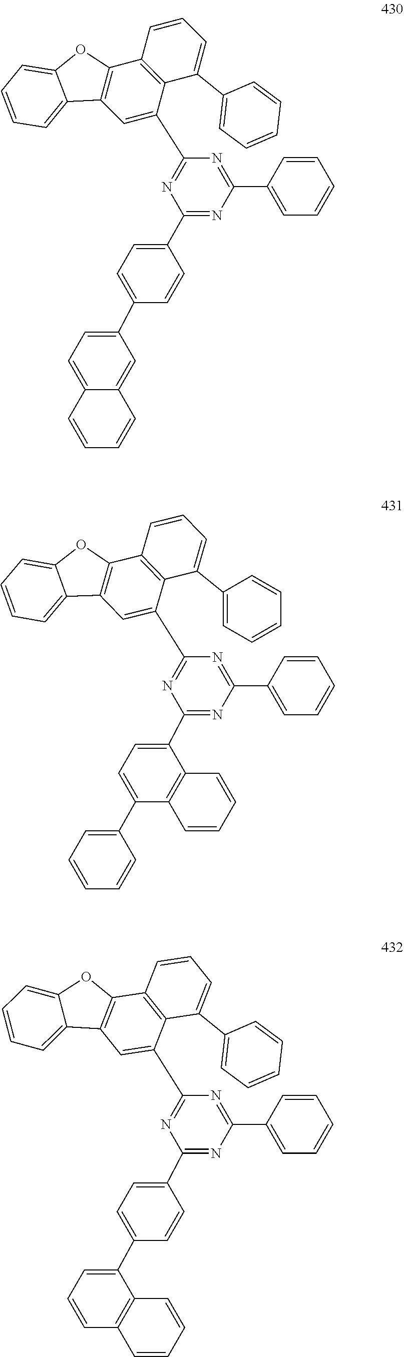 [SMILES: c1ccc(-c2nc(-c3ccc(-c4ccc5ccccc5c4)cc3)nc(-c3cc4c5ccccc5oc4c4cccc(-c5ccccc5)c34)n2)cc1 ; c1ccc(-c2nc(-c3ccc(-c4ccccc4)c4ccccc34)nc(-c3cc4c5ccccc5oc4c4cccc(-c5ccccc5)c34)n2)cc1 ; c1ccc(-c2nc(-c3ccc(-c4cccc5ccccc45)cc3)nc(-c3cc4c5ccccc5oc4c4cccc(-c5ccccc5)c34)n2)cc1]

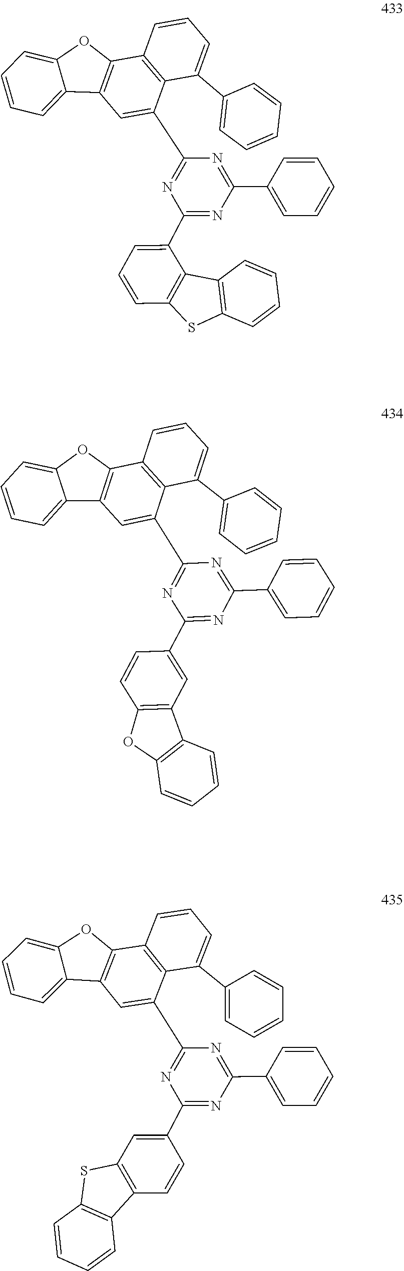 [SMILES: c1ccc(-c2nc(-c3cc4c5ccccc5oc4c4cccc(-c5ccccc5)c34)nc(-c3cccc4sc5ccccc5c34)n2)cc1 ; c1ccc(-c2nc(-c3ccc4oc5ccccc5c4c3)nc(-c3cc4c5ccccc5oc4c4cccc(-c5ccccc5)c34)n2)cc1 ; c1ccc(-c2nc(-c3ccc4c(c3)sc3ccccc34)nc(-c3cc4c5ccccc5oc4c4cccc(-c5ccccc5)c34)n2)cc1]

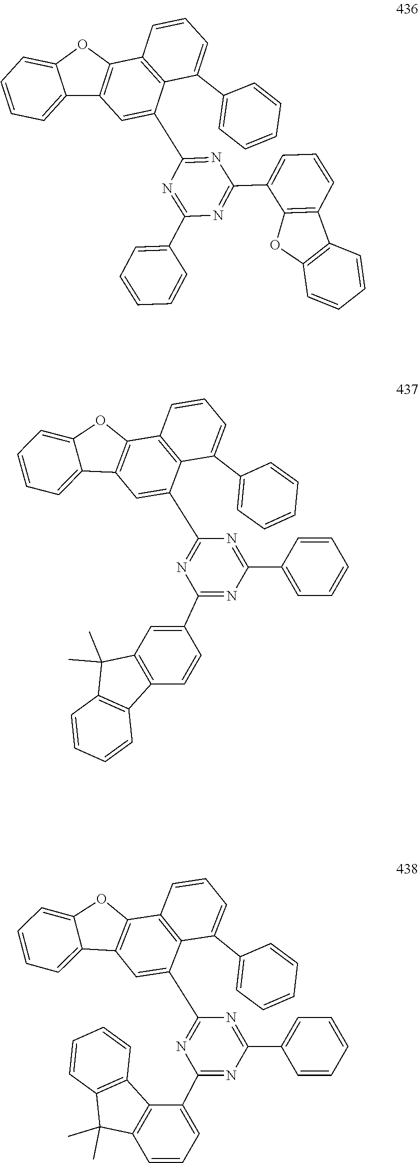 [SMILES: c1ccc(-c2nc(-c3cccc4c3oc3ccccc34)nc(-c3cc4c5ccccc5oc4c4cccc(-c5ccccc5)c34)n2)cc1 ; CC1(C)c2ccccc2-c2ccc(-c3nc(-c4ccccc4)nc(-c4cc5c6ccccc6oc5c5cccc(-c6ccccc6)c45)n3)cc21 ; CC1(C)c2ccccc2-c2c(-c3nc(-c4ccccc4)nc(-c4cc5c6ccccc6oc5c5cccc(-c6ccccc6)c45)n3)cccc21]

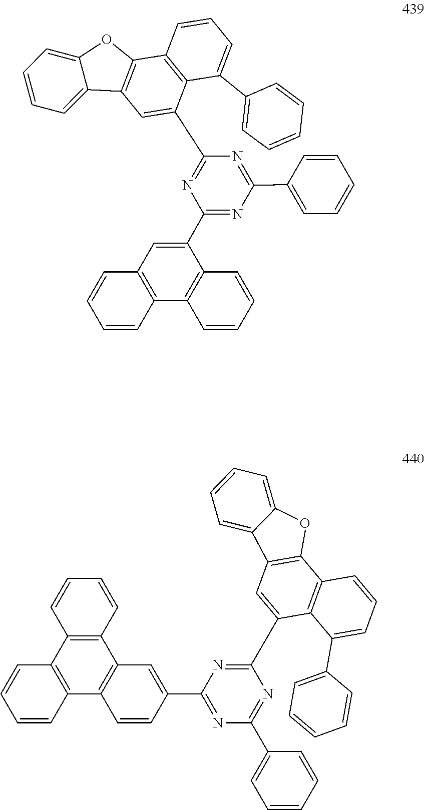 [SMILES: c1ccc(-c2nc(-c3cc4ccccc4c4ccccc34)nc(-c3cc4c5ccccc5oc4c4cccc(-c5ccccc5)c34)n2)cc1 ; c1ccc(-c2nc(-c3ccc4c5ccccc5c5ccccc5c4c3)nc(-c3cc4c5ccccc5oc4c4cccc(-c5ccccc5)c34)n2)cc1]

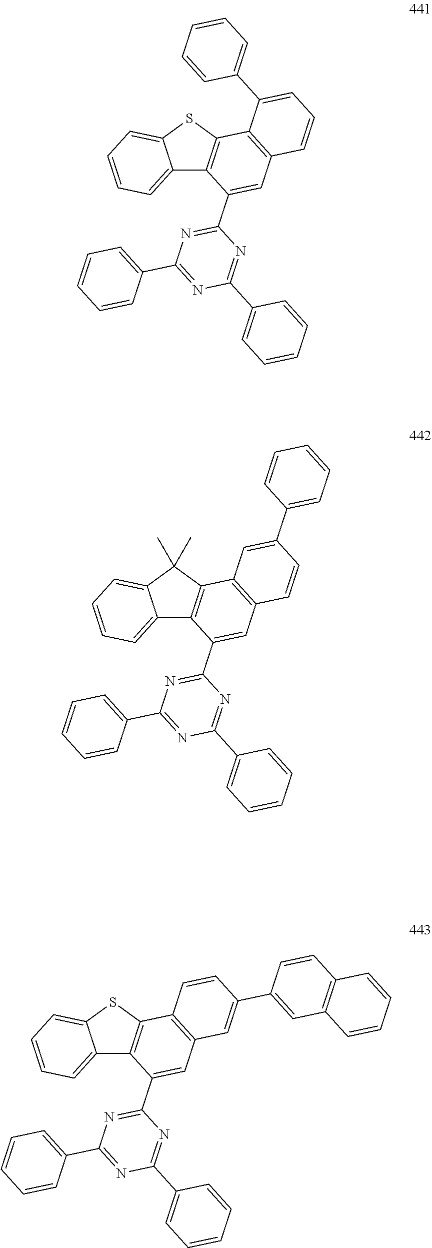 [SMILES: c1ccc(-c2nc(-c3ccccc3)nc(-c3cc4cccc(-c5ccccc5)c4c4sc5ccccc5c34)n2)cc1 ; CC1(C)c2ccccc2-c2c(-c3nc(-c4ccccc4)nc(-c4ccccc4)n3)cc3ccc(-c4ccccc4)cc3c21 ; c1ccc(-c2nc(-c3ccccc3)nc(-c3cc4cc(-c5ccc6ccccc6c5)ccc4c4sc5ccccc5c34)n2)cc1]

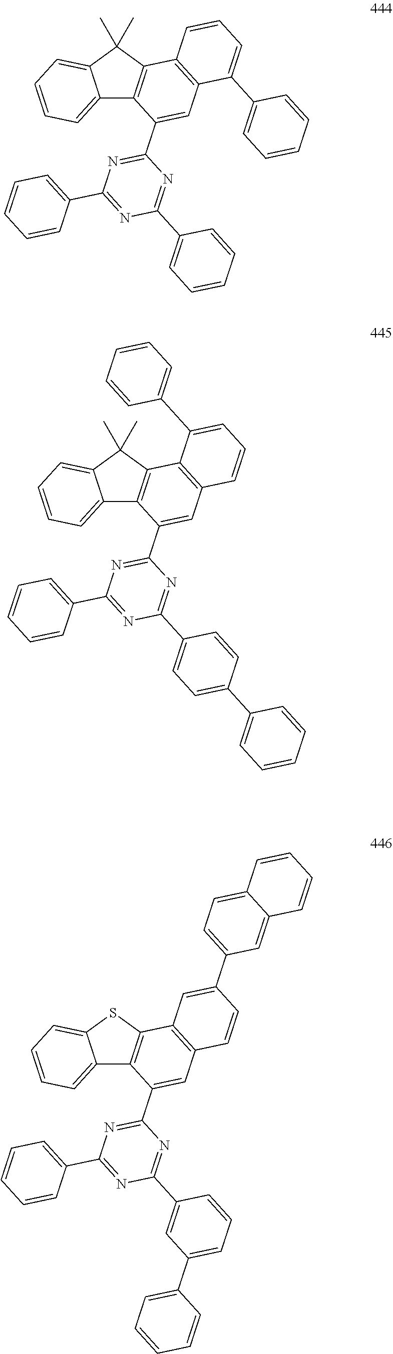 [SMILES: CC1(C)c2ccccc2-c2c(-c3nc(-c4ccccc4)nc(-c4ccccc4)n3)cc3c(-c4ccccc4)cccc3c21 ; CC1(C)c2ccccc2-c2c(-c3nc(-c4ccccc4)nc(-c4ccc(-c5ccccc5)cc4)n3)cc3cccc(-c4ccccc4)c3c21 ; c1ccc(-c2cccc(-c3nc(-c4ccccc4)nc(-c4cc5ccc(-c6ccc7ccccc7c6)cc5c5sc6ccccc6c45)n3)c2)cc1]

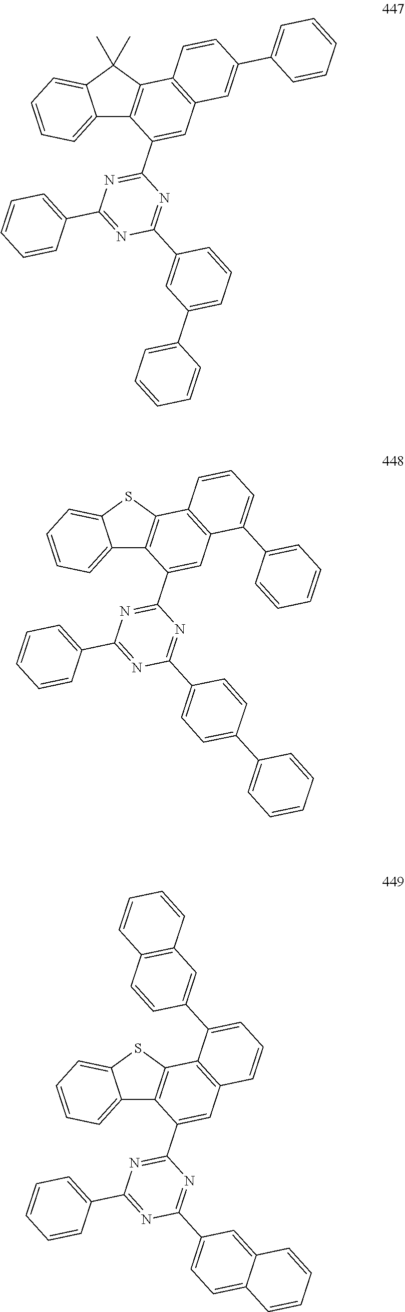 [SMILES: CC1(C)c2ccccc2-c2c(-c3nc(-c4ccccc4)nc(-c4cccc(-c5ccccc5)c4)n3)cc3cc(-c4ccccc4)ccc3c21 ; c1ccc(-c2ccc(-c3nc(-c4ccccc4)nc(-c4cc5c(-c6ccccc6)cccc5c5sc6ccccc6c45)n3)cc2)cc1 ; c1ccc(-c2nc(-c3ccc4ccccc4c3)nc(-c3cc4cccc(-c5ccc6ccccc6c5)c4c4sc5ccccc5c34)n2)cc1]

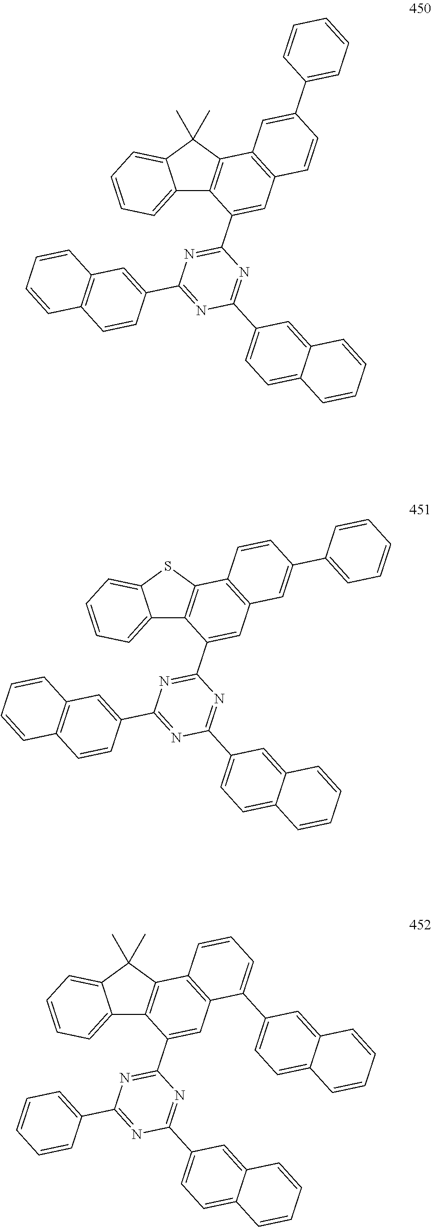 [SMILES: CC1(C)c2ccccc2-c2c(-c3nc(-c4ccc5ccccc5c4)nc(-c4ccc5ccccc5c4)n3)cc3ccc(-c4ccccc4)cc3c21 ; c1ccc(-c2ccc3c(c2)cc(-c2nc(-c4ccc5ccccc5c4)nc(-c4ccc5ccccc5c4)n2)c2c4ccccc4sc32)cc1 ; CC1(C)c2ccccc2-c2c(-c3nc(-c4ccccc4)nc(-c4ccc5ccccc5c4)n3)cc3c(-c4ccc5ccccc5c4)cccc3c21]

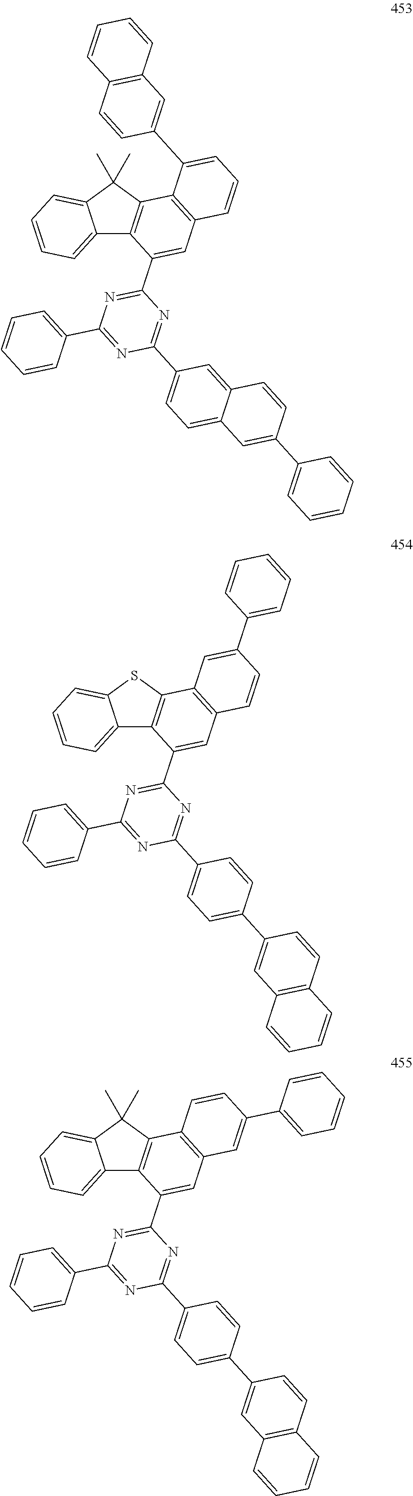

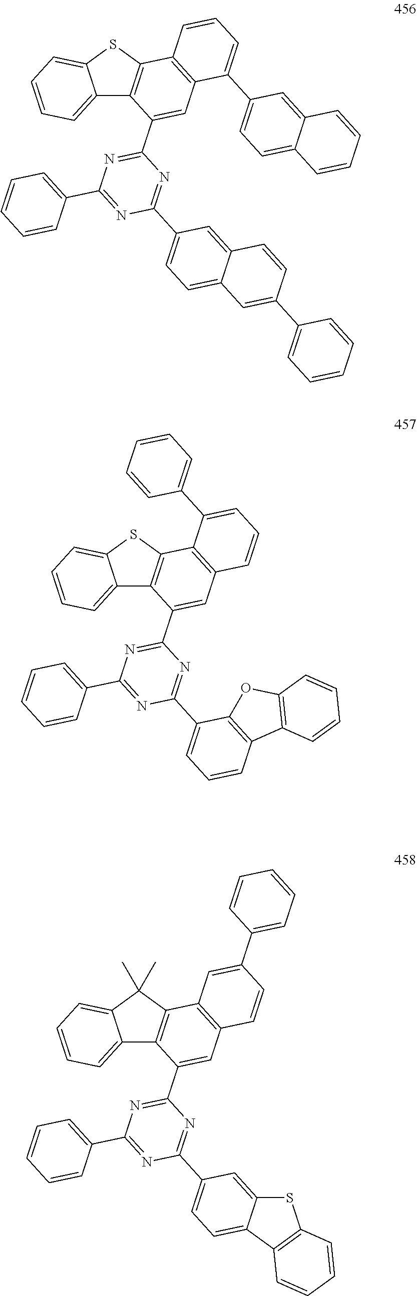 [SMILES: c1ccc(-c2ccc3cc(-c4nc(-c5ccccc5)nc(-c5cc6c(-c7ccc8ccccc8c7)cccc6c6sc7ccccc7c56)n4)ccc3c2)cc1 ; c1ccc(-c2nc(-c3cccc4c3oc3ccccc34)nc(-c3cc4cccc(-c5ccccc5)c4c4sc5ccccc5c34)n2)cc1 ; CC1(C)c2ccccc2-c2c(-c3nc(-c4ccccc4)nc(-c4ccc5c(c4)sc4ccccc45)n3)cc3ccc(-c4ccccc4)cc3c21]

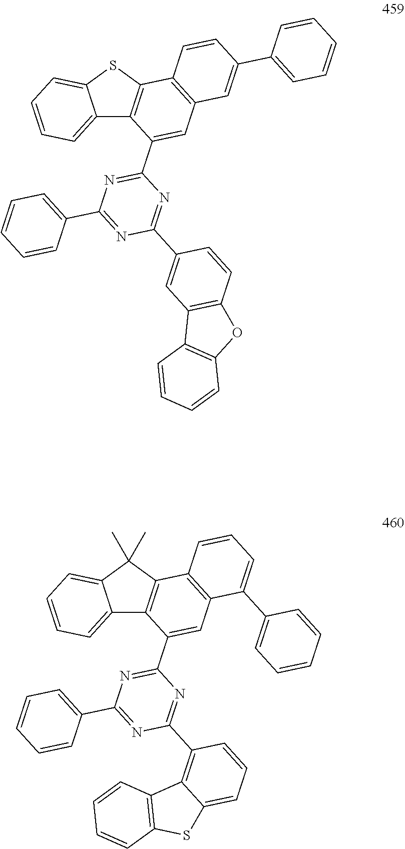 [SMILES: c1ccc(-c2ccc3c(c2)cc(-c2nc(-c4ccccc4)nc(-c4ccc5oc6ccccc6c5c4)n2)c2c4ccccc4sc32)cc1 ; CC1(C)c2ccccc2-c2c(-c3nc(-c4ccccc4)nc(-c4cccc5sc6ccccc6c45)n3)cc3c(-c4ccccc4)cccc3c21]

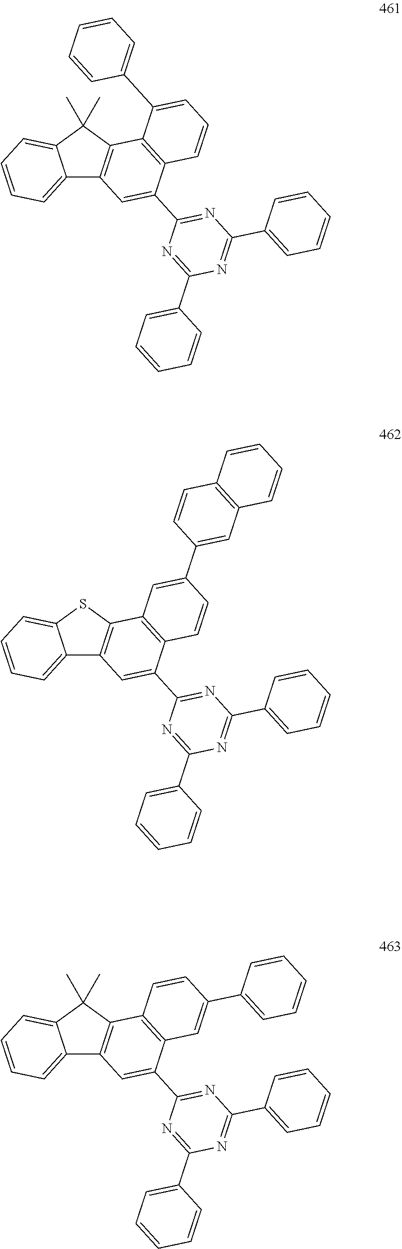 [SMILES: CC1(C)c2ccccc2-c2cc(-c3nc(-c4ccccc4)nc(-c4ccccc4)n3)c3cccc(-c4ccccc4)c3c21 ; c1ccc(-c2nc(-c3ccccc3)nc(-c3cc4c5ccccc5sc4c4cc(-c5ccc6ccccc6c5)ccc34)n2)cc1 ; CC1(C)c2ccccc2-c2cc(-c3nc(-c4ccccc4)nc(-c4ccccc4)n3)c3cc(-c4ccccc4)ccc3c21]

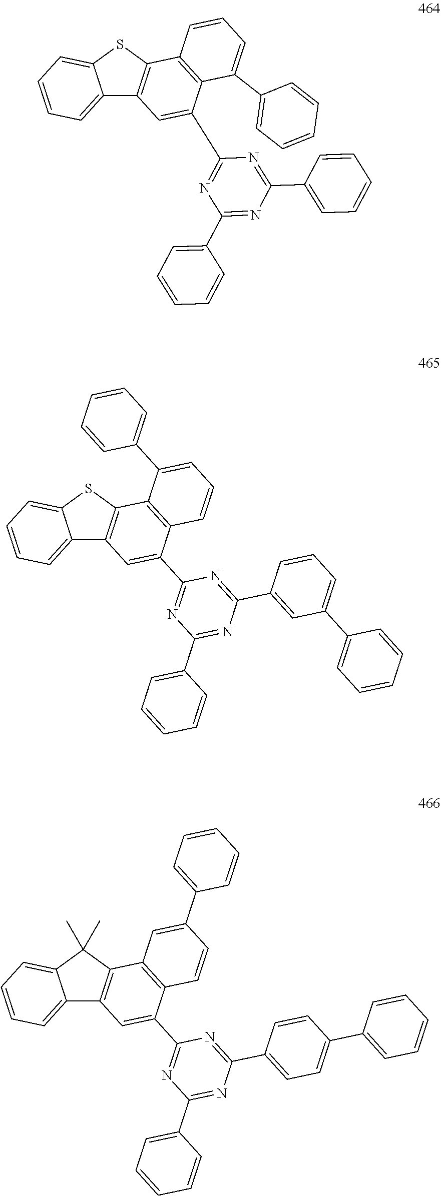 [SMILES: c1ccc(-c2nc(-c3ccccc3)nc(-c3cc4c5ccccc5sc4c4cccc(-c5ccccc5)c34)n2)cc1 ; c1ccc(-c2cccc(-c3nc(-c4ccccc4)nc(-c4cc5c6ccccc6sc5c5c(-c6ccccc6)cccc45)n3)c2)cc1 ; CC1(C)c2ccccc2-c2cc(-c3nc(-c4ccccc4)nc(-c4ccc(-c5ccccc5)cc4)n3)c3ccc(-c4ccccc4)cc3c21]

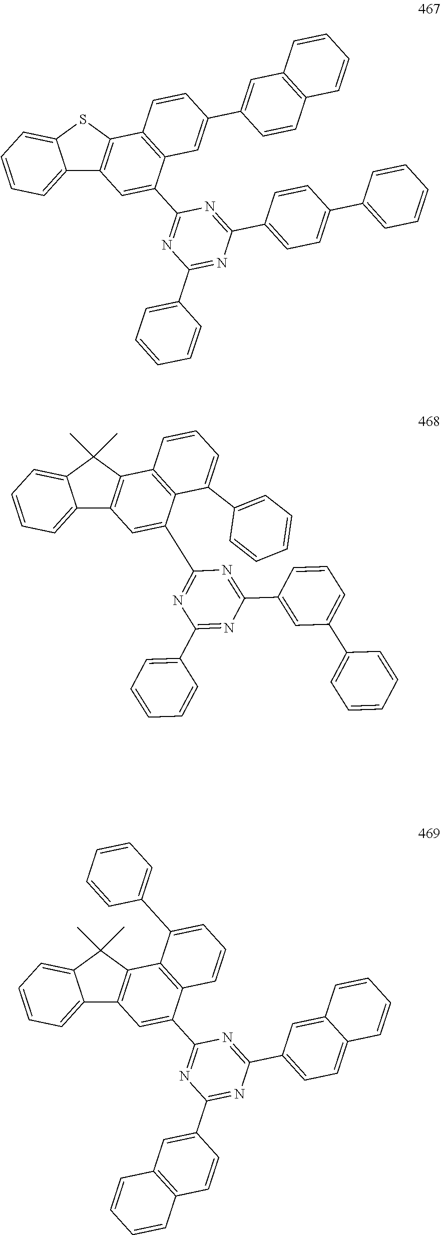 [SMILES: c1ccc(-c2ccc(-c3nc(-c4ccccc4)nc(-c4cc5c6ccccc6sc5c5ccc(-c6ccc7ccccc7c6)cc45)n3)cc2)cc1 ; CC1(C)c2ccccc2-c2cc(-c3nc(-c4ccccc4)nc(-c4cccc(-c5ccccc5)c4)n3)c3c(-c4ccccc4)cccc3c21 ; CC1(C)c2ccccc2-c2cc(-c3nc(-c4ccc5ccccc5c4)nc(-c4ccc5ccccc5c4)n3)c3cccc(-c4ccccc4)c3c21]

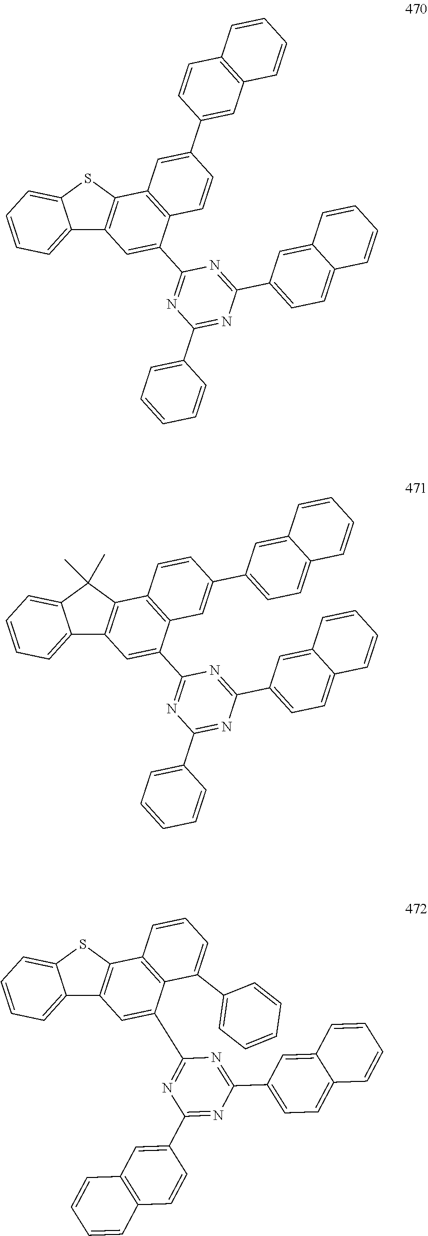 [SMILES: c1ccc(-c2nc(-c3ccc4ccccc4c3)nc(-c3cc4c5ccccc5sc4c4cc(-c5ccc6ccccc6c5)ccc34)n2)cc1 ; CC1(C)c2ccccc2-c2cc(-c3nc(-c4ccccc4)nc(-c4ccc5ccccc5c4)n3)c3cc(-c4ccc5ccccc5c4)ccc3c21 ; c1ccc(-c2cccc3c2c(-c2nc(-c4ccc5ccccc5c4)nc(-c4ccc5ccccc5c4)n2)cc2c4ccccc4sc32)cc1]

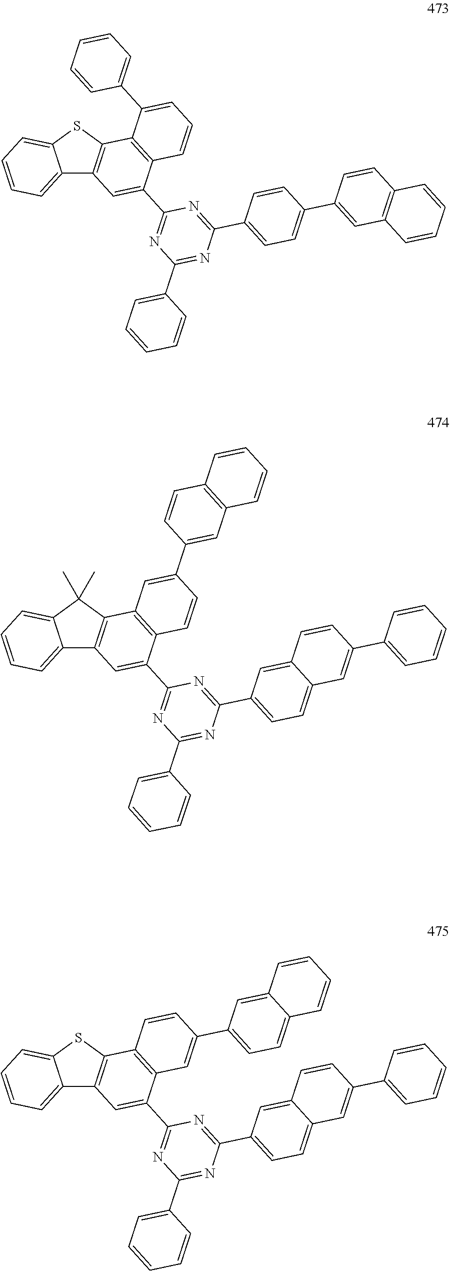 [SMILES: c1ccc(-c2nc(-c3ccc(-c4ccc5ccccc5c4)cc3)nc(-c3cc4c5ccccc5sc4c4c(-c5ccccc5)cccc34)n2)cc1 ; CC1(C)c2ccccc2-c2cc(-c3nc(-c4ccccc4)nc(-c4ccc5cc(-c6ccccc6)ccc5c4)n3)c3ccc(-c4ccc5ccccc5c4)cc3c21 ; c1ccc(-c2ccc3cc(-c4nc(-c5ccccc5)nc(-c5cc6c7ccccc7sc6c6ccc(-c7ccc8ccccc8c7)cc56)n4)ccc3c2)cc1]

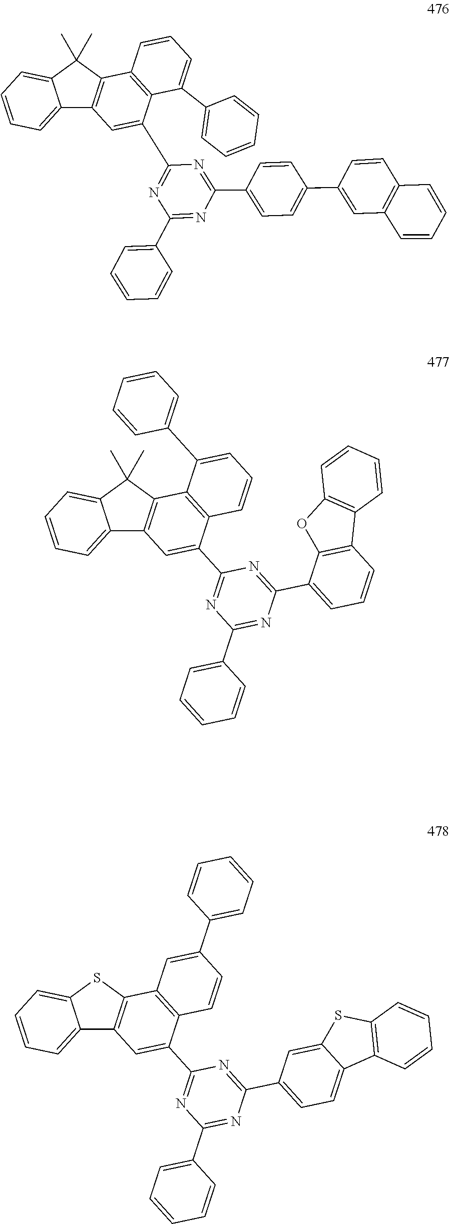 [SMILES: CC1(C)c2ccccc2-c2cc(-c3nc(-c4ccccc4)nc(-c4ccc(-c5ccc6ccccc6c5)cc4)n3)c3c(-c4ccccc4)cccc3c21 ; CC1(C)c2ccccc2-c2cc(-c3nc(-c4ccccc4)nc(-c4cccc5c4oc4ccccc45)n3)c3cccc(-c4ccccc4)c3c21 ; c1ccc(-c2ccc3c(-c4nc(-c5ccccc5)nc(-c5ccc6c(c5)sc5ccccc56)n4)cc4c5ccccc5sc4c3c2)cc1]

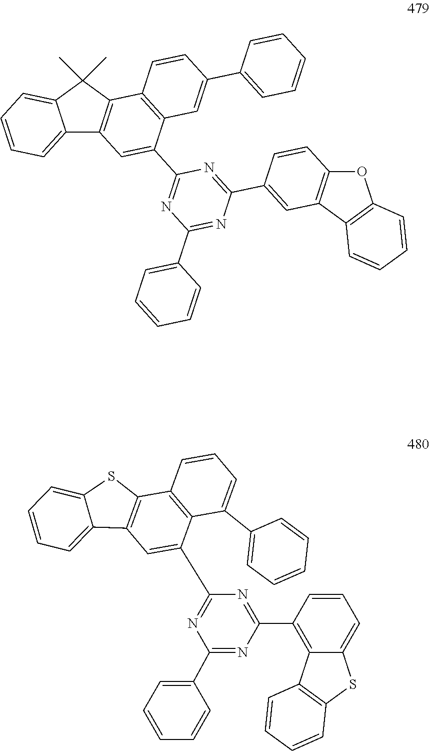 [SMILES: CC1(C)c2ccccc2-c2cc(-c3nc(-c4ccccc4)nc(-c4ccc5oc6ccccc6c5c4)n3)c3cc(-c4ccccc4)ccc3c21 ; c1ccc(-c2nc(-c3cc4c5ccccc5sc4c4cccc(-c5ccccc5)c34)nc(-c3cccc4sc5ccccc5c34)n2)cc1]

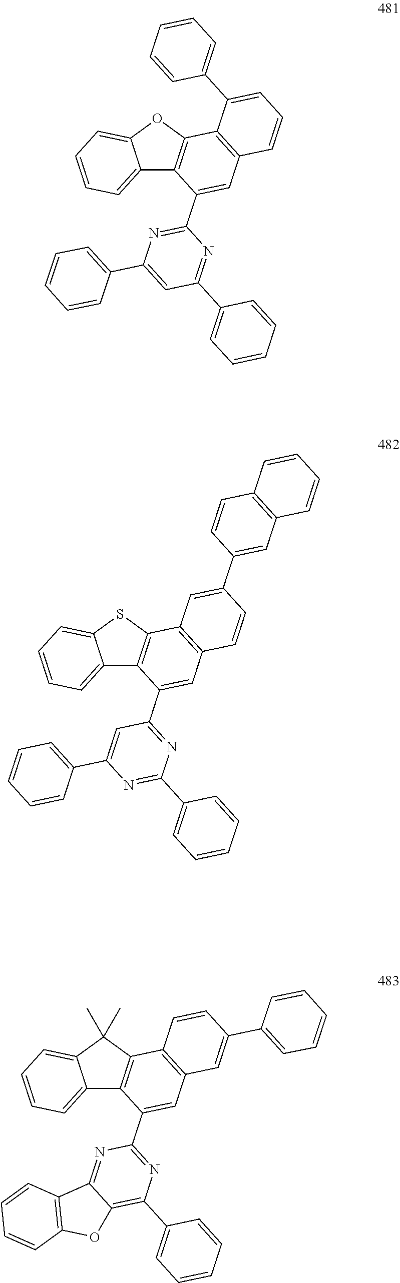 [SMILES: c1ccc(-c2cc(-c3ccccc3)nc(-c3cc4cccc(-c5ccccc5)c4c4oc5ccccc5c34)n2)cc1 ; c1ccc(-c2cc(-c3cc4ccc(-c5ccc6ccccc6c5)cc4c4sc5ccccc5c34)nc(-c3ccccc3)n2)cc1 ; CC1(C)c2ccccc2-c2c(-c3nc(-c4ccccc4)c4oc5ccccc5c4n3)cc3cc(-c4ccccc4)ccc3c21]

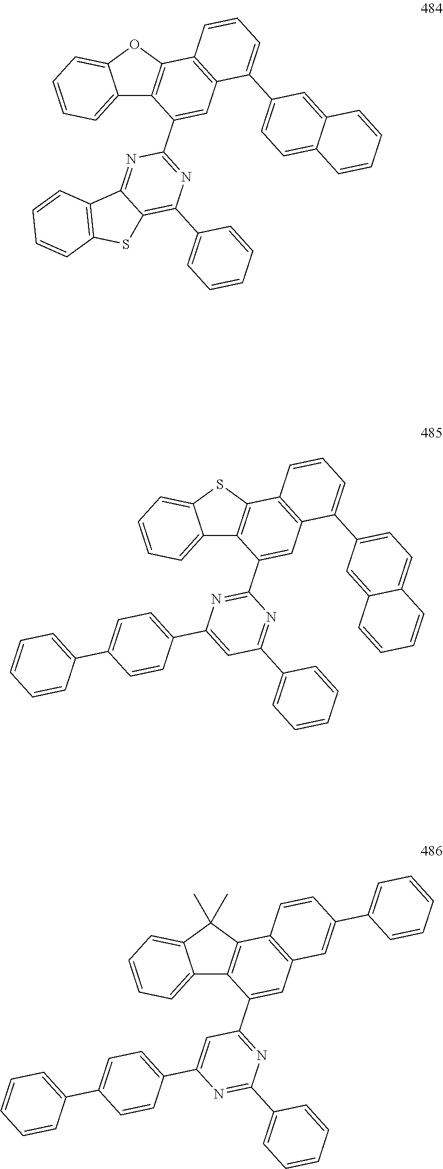 [SMILES: c1ccc(-c2nc(-c3cc4c(-c5ccc6ccccc6c5)cccc4c4oc5ccccc5c34)nc3c2sc2ccccc23)cc1 ; c1ccc(-c2ccc(-c3cc(-c4ccccc4)nc(-c4cc5c(-c6ccc7ccccc7c6)cccc5c5sc6ccccc6c45)n3)cc2)cc1 ; CC1(C)c2ccccc2-c2c(-c3cc(-c4ccc(-c5ccccc5)cc4)nc(-c4ccccc4)n3)cc3cc(-c4ccccc4)ccc3c21]

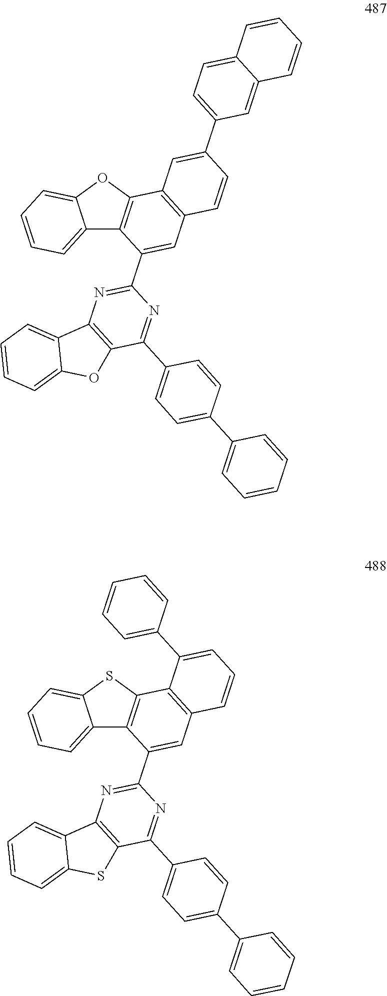 [SMILES: c1ccc(-c2ccc(-c3nc(-c4cc5ccc(-c6ccc7ccccc7c6)cc5c5oc6ccccc6c45)nc4c3oc3ccccc34)cc2)cc1 ; c1ccc(-c2ccc(-c3nc(-c4cc5cccc(-c6ccccc6)c5c5sc6ccccc6c45)nc4c3sc3ccccc34)cc2)cc1]

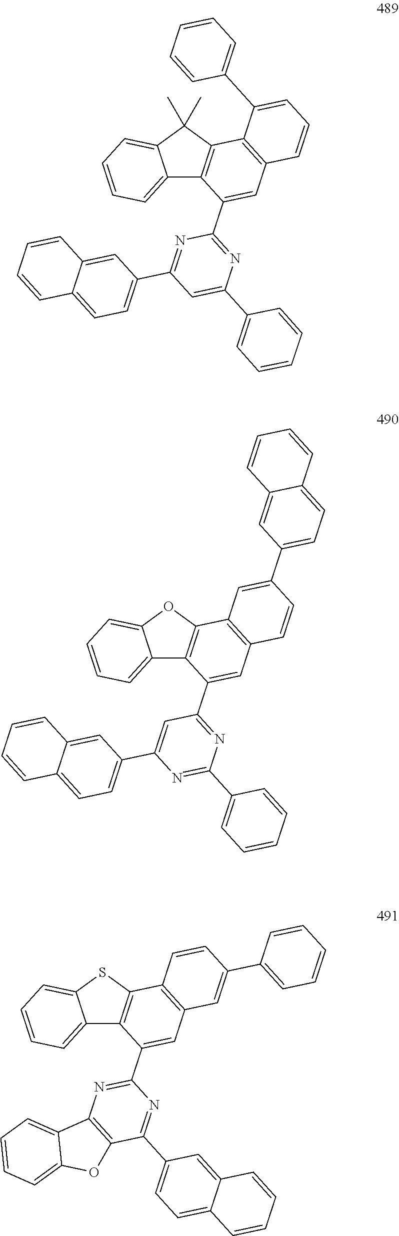 [SMILES: CC1(C)c2ccccc2-c2c(-c3nc(-c4ccccc4)cc(-c4ccc5ccccc5c4)n3)cc3cccc(-c4ccccc4)c3c21 ; c1ccc(-c2nc(-c3ccc4ccccc4c3)cc(-c3cc4ccc(-c5ccc6ccccc6c5)cc4c4oc5ccccc5c34)n2)cc1 ; c1ccc(-c2ccc3c(c2)cc(-c2nc(-c4ccc5ccccc5c4)c4oc5ccccc5c4n2)c2c4ccccc4sc32)cc1]

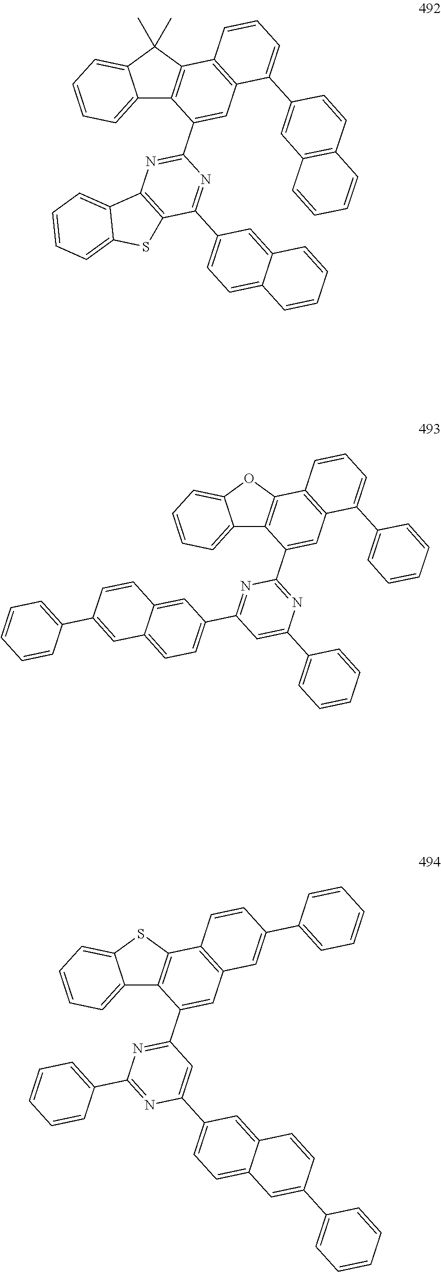 [SMILES: CC1(C)c2ccccc2-c2c(-c3nc(-c4ccc5ccccc5c4)c4sc5ccccc5c4n3)cc3c(-c4ccc5ccccc5c4)cccc3c21 ; c1ccc(-c2ccc3cc(-c4cc(-c5ccccc5)nc(-c5cc6c(-c7ccccc7)cccc6c6oc7ccccc7c56)n4)ccc3c2)cc1 ; c1ccc(-c2ccc3cc(-c4cc(-c5cc6cc(-c7ccccc7)ccc6c6sc7ccccc7c56)nc(-c5ccccc5)n4)ccc3c2)cc1]

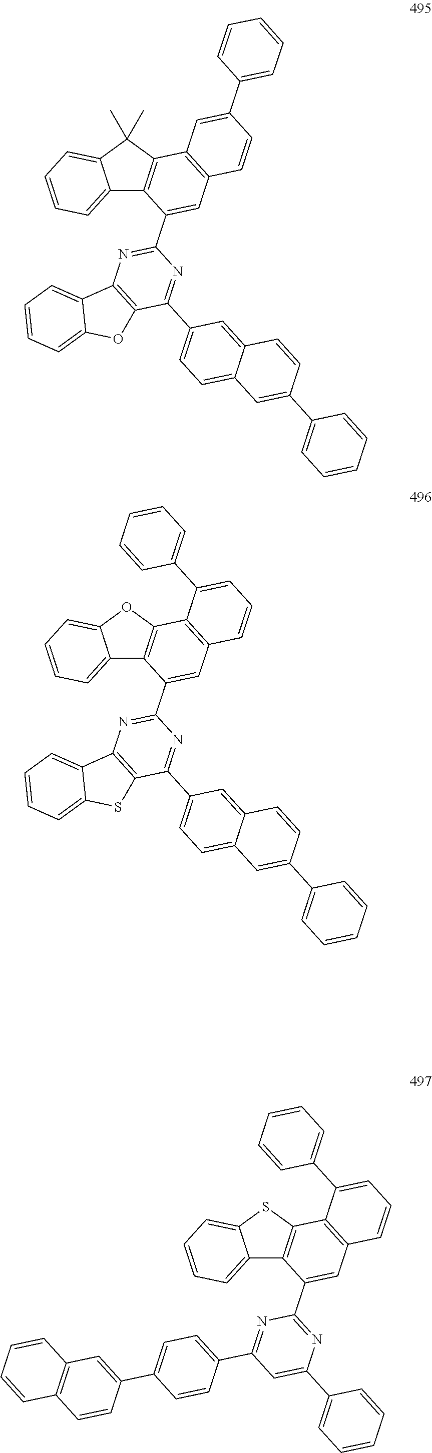 [SMILES: CC1(C)c2ccccc2-c2c(-c3nc(-c4ccc5cc(-c6ccccc6)ccc5c4)c4oc5ccccc5c4n3)cc3ccc(-c4ccccc4)cc3c21 ; c1ccc(-c2ccc3cc(-c4nc(-c5cc6cccc(-c7ccccc7)c6c6oc7ccccc7c56)nc5c4sc4ccccc45)ccc3c2)cc1 ; c1ccc(-c2cc(-c3ccc(-c4ccc5ccccc5c4)cc3)nc(-c3cc4cccc(-c5ccccc5)c4c4sc5ccccc5c34)n2)cc1]

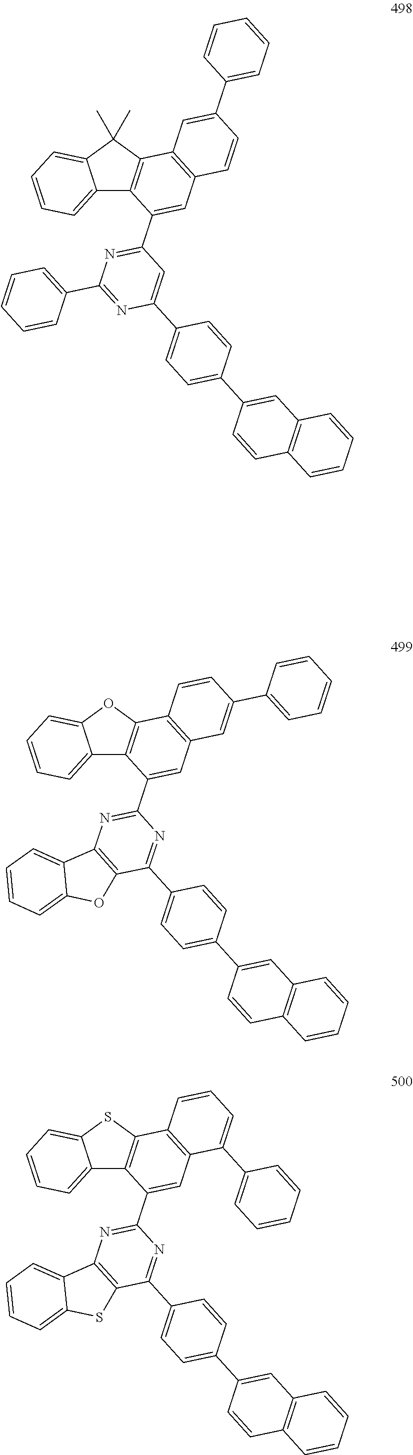 [SMILES: CC1(C)c2ccccc2-c2c(-c3cc(-c4ccc(-c5ccc6ccccc6c5)cc4)nc(-c4ccccc4)n3)cc3ccc(-c4ccccc4)cc3c21 ; c1ccc(-c2ccc3c(c2)cc(-c2nc(-c4ccc(-c5ccc6ccccc6c5)cc4)c4oc5ccccc5c4n2)c2c4ccccc4oc32)cc1 ; c1ccc(-c2cccc3c2cc(-c2nc(-c4ccc(-c5ccc6ccccc6c5)cc4)c4sc5ccccc5c4n2)c2c4ccccc4sc32)cc1]

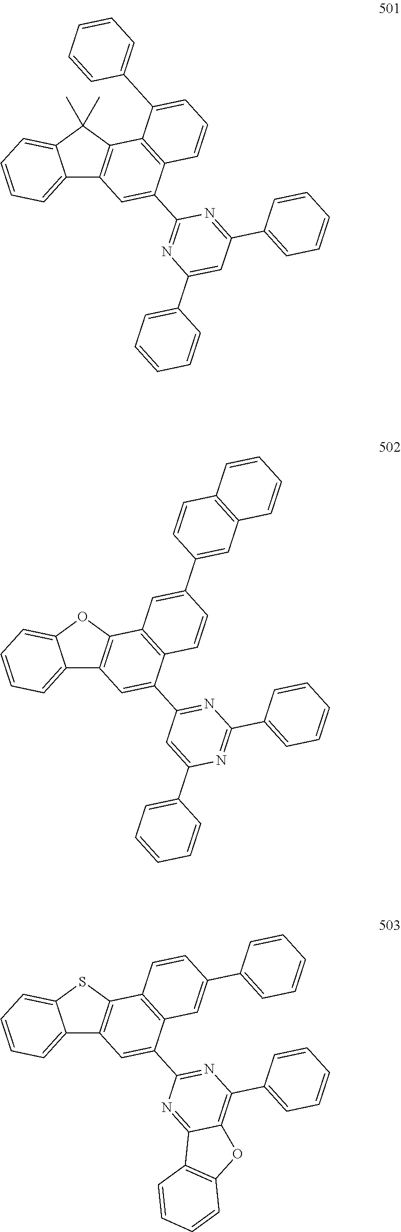 [SMILES: CC1(C)c2ccccc2-c2cc(-c3nc(-c4ccccc4)cc(-c4ccccc4)n3)c3cccc(-c4ccccc4)c3c21 ; c1ccc(-c2cc(-c3cc4c5ccccc5oc4c4cc(-c5ccc6ccccc6c5)ccc34)nc(-c3ccccc3)n2)cc1 ; c1ccc(-c2ccc3c(c2)c(-c2nc(-c4ccccc4)c4oc5ccccc5c4n2)cc2c4ccccc4sc32)cc1]

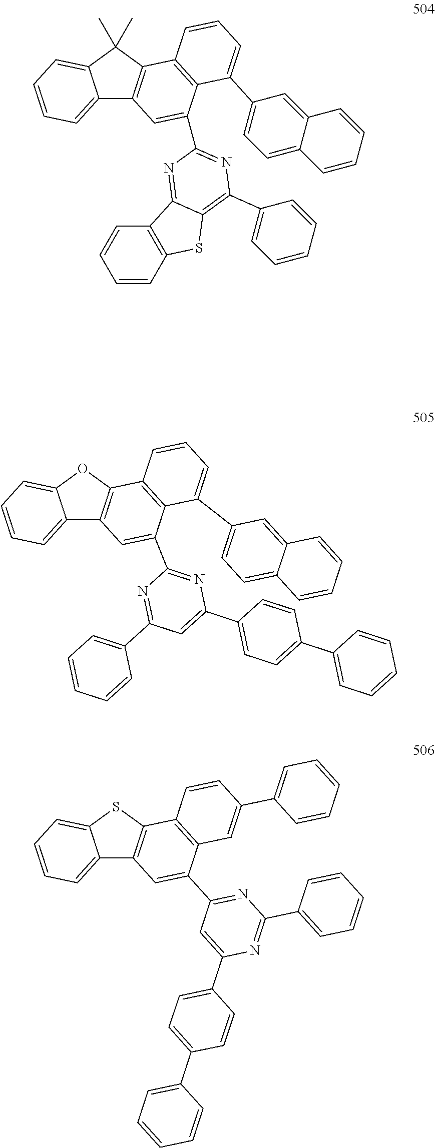 [SMILES: CC1(C)c2ccccc2-c2cc(-c3nc(-c4ccccc4)c4sc5ccccc5c4n3)c3c(-c4ccc5ccccc5c4)cccc3c21 ; c1ccc(-c2ccc(-c3cc(-c4ccccc4)nc(-c4cc5c6ccccc6oc5c5cccc(-c6ccc7ccccc7c6)c45)n3)cc2)cc1 ; c1ccc(-c2ccc(-c3cc(-c4cc5c6ccccc6sc5c5ccc(-c6ccccc6)cc45)nc(-c4ccccc4)n3)cc2)cc1]

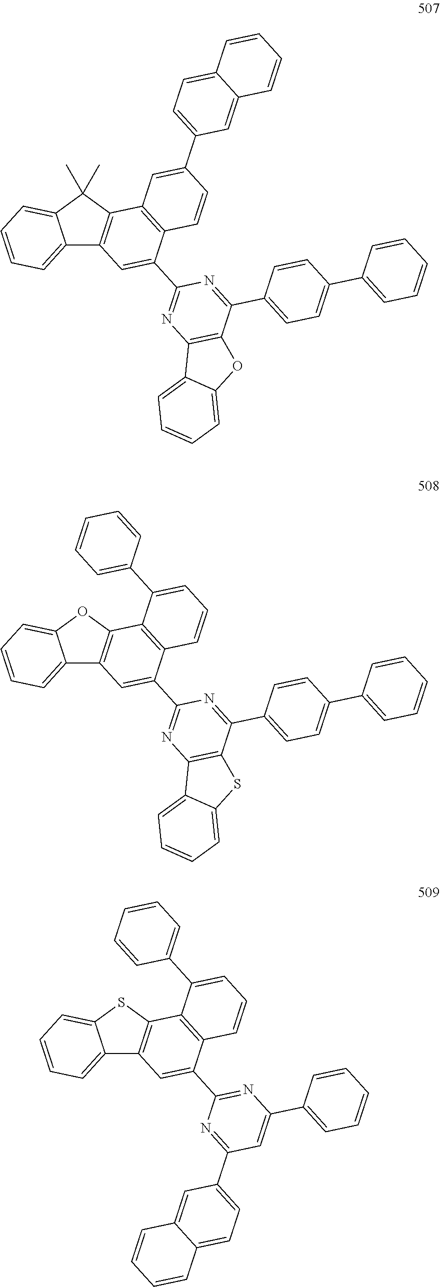 [SMILES: CC1(C)c2ccccc2-c2cc(-c3nc(-c4ccc(-c5ccccc5)cc4)c4oc5ccccc5c4n3)c3ccc(-c4ccc5ccccc5c4)cc3c21 ; c1ccc(-c2ccc(-c3nc(-c4cc5c6ccccc6oc5c5c(-c6ccccc6)cccc45)nc4c3sc3ccccc34)cc2)cc1 ; c1ccc(-c2cc(-c3ccc4ccccc4c3)nc(-c3cc4c5ccccc5sc4c4c(-c5ccccc5)cccc34)n2)cc1]

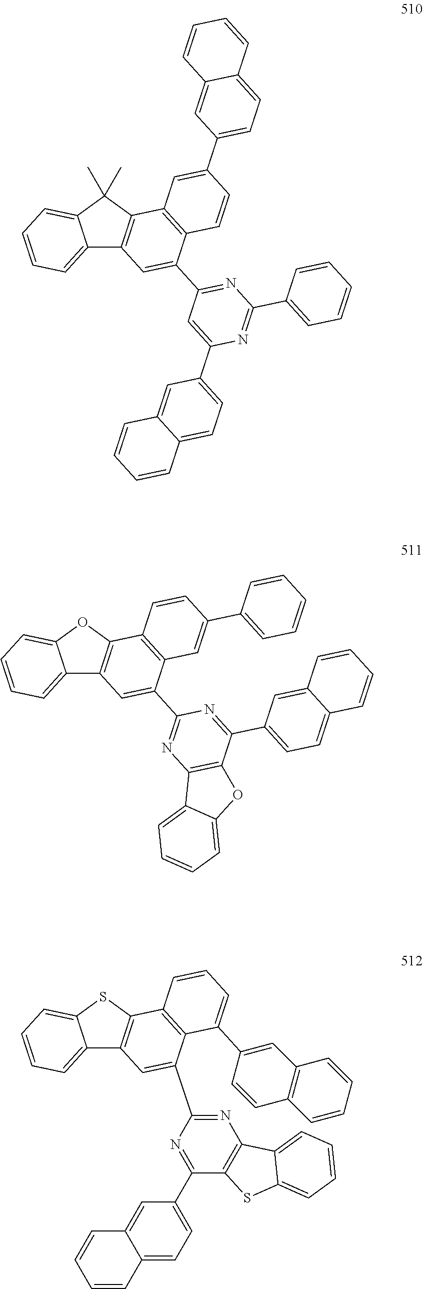 [SMILES: CC1(C)c2ccccc2-c2cc(-c3cc(-c4ccc5ccccc5c4)nc(-c4ccccc4)n3)c3ccc(-c4ccc5ccccc5c4)cc3c21 ; c1ccc(-c2ccc3c(c2)c(-c2nc(-c4ccc5ccccc5c4)c4oc5ccccc5c4n2)cc2c4ccccc4oc32)cc1 ; c1ccc2cc(-c3nc(-c4cc5c6ccccc6sc5c5cccc(-c6ccc7ccccc7c6)c45)nc4c3sc3ccccc34)ccc2c1]

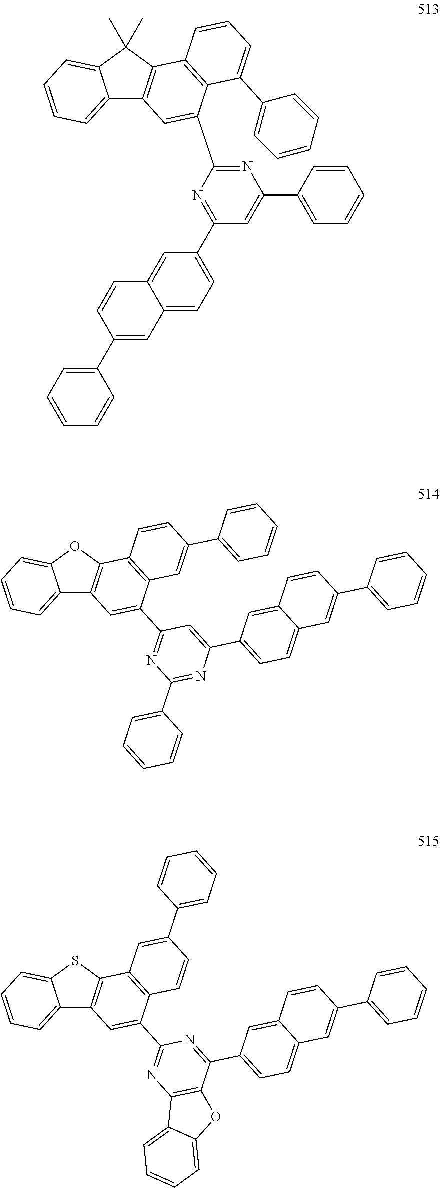 [SMILES: CC1(C)c2ccccc2-c2cc(-c3nc(-c4ccccc4)cc(-c4ccc5cc(-c6ccccc6)ccc5c4)n3)c3c(-c4ccccc4)cccc3c21 ; c1ccc(-c2ccc3cc(-c4cc(-c5cc6c7ccccc7oc6c6ccc(-c7ccccc7)cc56)nc(-c5ccccc5)n4)ccc3c2)cc1 ; c1ccc(-c2ccc3cc(-c4nc(-c5cc6c7ccccc7sc6c6cc(-c7ccccc7)ccc56)nc5c4oc4ccccc45)ccc3c2)cc1]

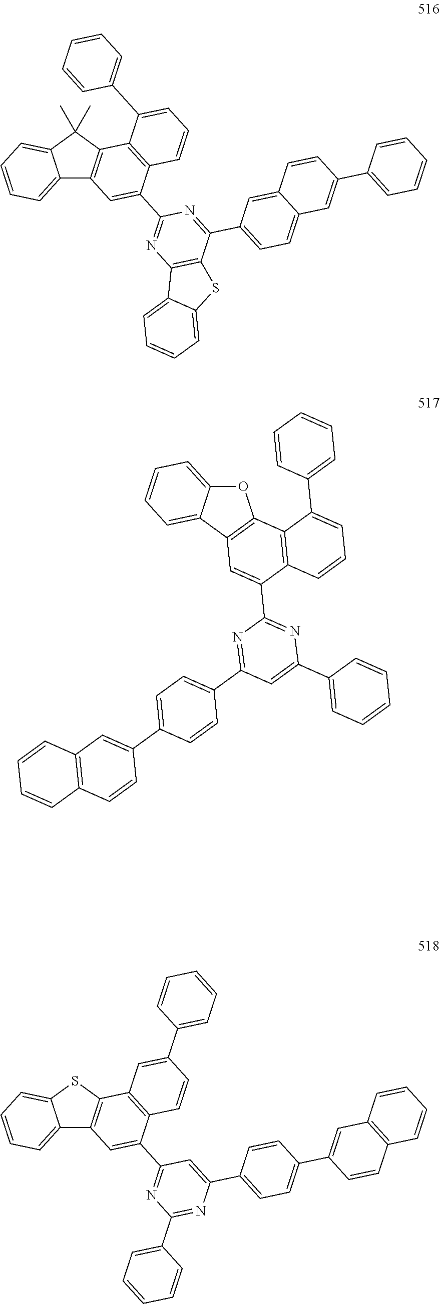 [SMILES: CC1(C)c2ccccc2-c2cc(-c3nc(-c4ccc5cc(-c6ccccc6)ccc5c4)c4sc5ccccc5c4n3)c3cccc(-c4ccccc4)c3c21 ; c1ccc(-c2cc(-c3ccc(-c4ccc5ccccc5c4)cc3)nc(-c3cc4c5ccccc5oc4c4c(-c5ccccc5)cccc34)n2)cc1 ; c1ccc(-c2ccc3c(-c4cc(-c5ccc(-c6ccc7ccccc7c6)cc5)nc(-c5ccccc5)n4)cc4c5ccccc5sc4c3c2)cc1]

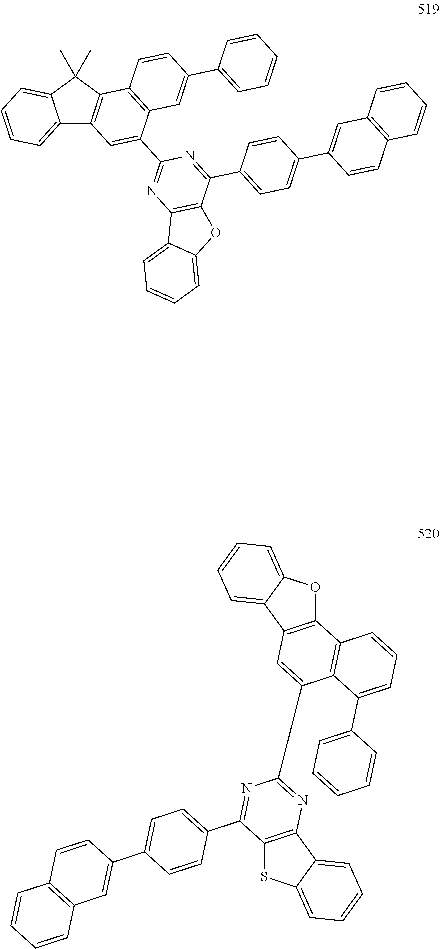 [SMILES: CC1(C)c2ccccc2-c2cc(-c3nc(-c4ccc(-c5ccc6ccccc6c5)cc4)c4oc5ccccc5c4n3)c3cc(-c4ccccc4)ccc3c21 ; c1ccc(-c2cccc3c2c(-c2nc(-c4ccc(-c5ccc6ccccc6c5)cc4)c4sc5ccccc5c4n2)cc2c4ccccc4oc32)cc1]

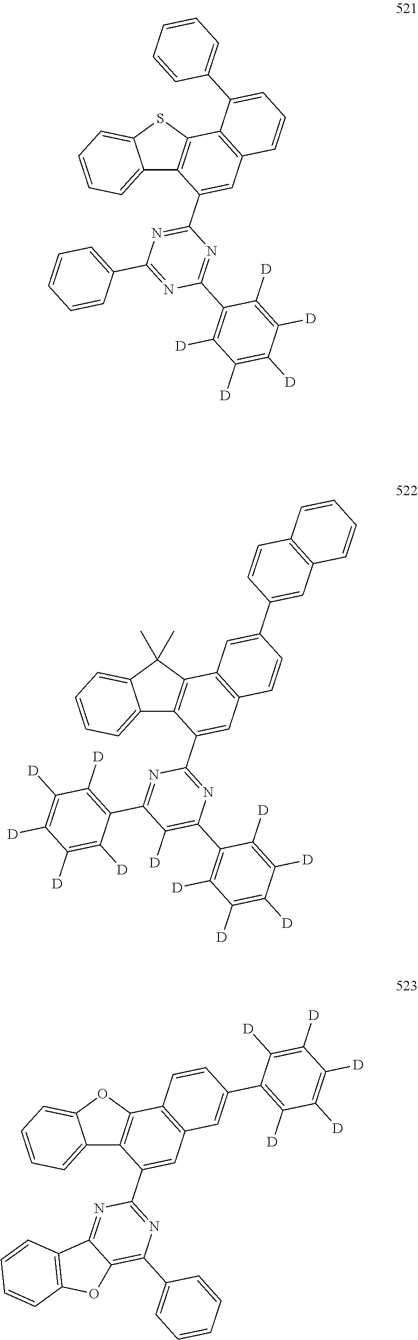 [SMILES: [2H]c1c([2H])c([2H])c(-c2nc(-c3ccccc3)nc(-c3cc4cccc(-c5ccccc5)c4c4sc5ccccc5c34)n2)c([2H])c1[2H] ; [2H]c1c(-c2c([2H])c([2H])c([2H])c([2H])c2[2H])nc(-c2cc3ccc(-c4ccc5ccccc5c4)cc3c3c2-c2ccccc2C3(C)C)nc1-c1c([2H])c([2H])c([2H])c([2H])c1[2H] ; [2H]c1c([2H])c([2H])c(-c2ccc3c(c2)cc(-c2nc(-c4ccccc4)c4oc5ccccc5c4n2)c2c4ccccc4oc32)c([2H])c1[2H]]

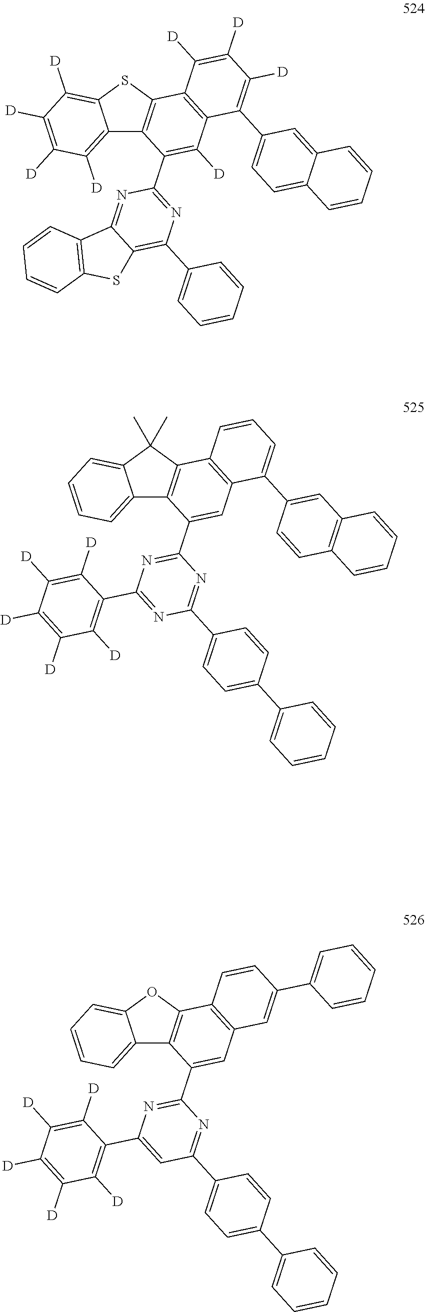 [SMILES: [2H]c1c([2H])c([2H])c2c(sc3c4c([2H])c([2H])c([2H])c(-c5ccc6ccccc6c5)c4c([2H])c(-c4nc(-c5ccccc5)c5sc6ccccc6c5n4)c32)c1[2H] ; [2H]c1c([2H])c([2H])c(-c2nc(-c3ccc(-c4ccccc4)cc3)nc(-c3cc4c(-c5ccc6ccccc6c5)cccc4c4c3-c3ccccc3C4(C)C)n2)c([2H])c1[2H] ; [2H]c1c([2H])c([2H])c(-c2cc(-c3ccc(-c4ccccc4)cc3)nc(-c3cc4cc(-c5ccccc5)ccc4c4oc5ccccc5c34)n2)c([2H])c1[2H]]

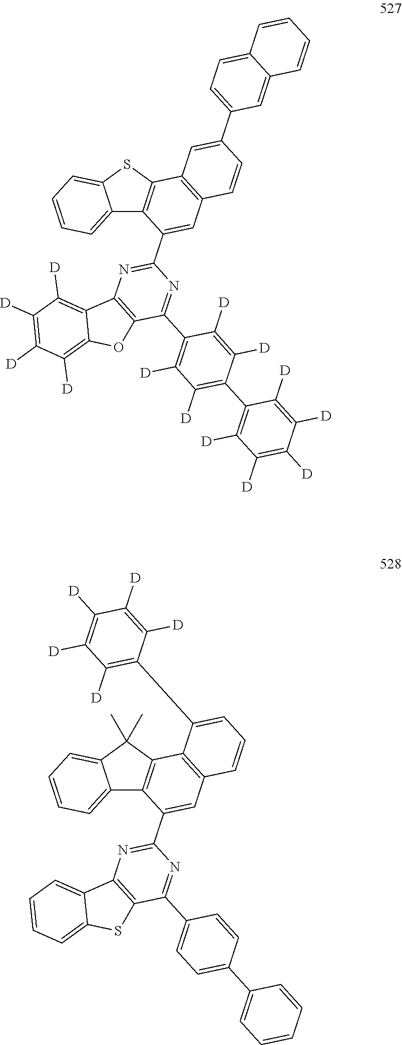 [SMILES: [2H]c1c([2H])c([2H])c(-c2c([2H])c([2H])c(-c3nc(-c4cc5ccc(-c6ccc7ccccc7c6)cc5c5sc6ccccc6c45)nc4c3oc3c([2H])c([2H])c([2H])c([2H])c34)c([2H])c2[2H])c([2H])c1[2H] ; [2H]c1c([2H])c([2H])c(-c2cccc3cc(-c4nc(-c5ccc(-c6ccccc6)cc5)c5sc6ccccc6c5n4)c4c(c23)C(C)(C)c2ccccc2-4)c([2H])c1[2H]]

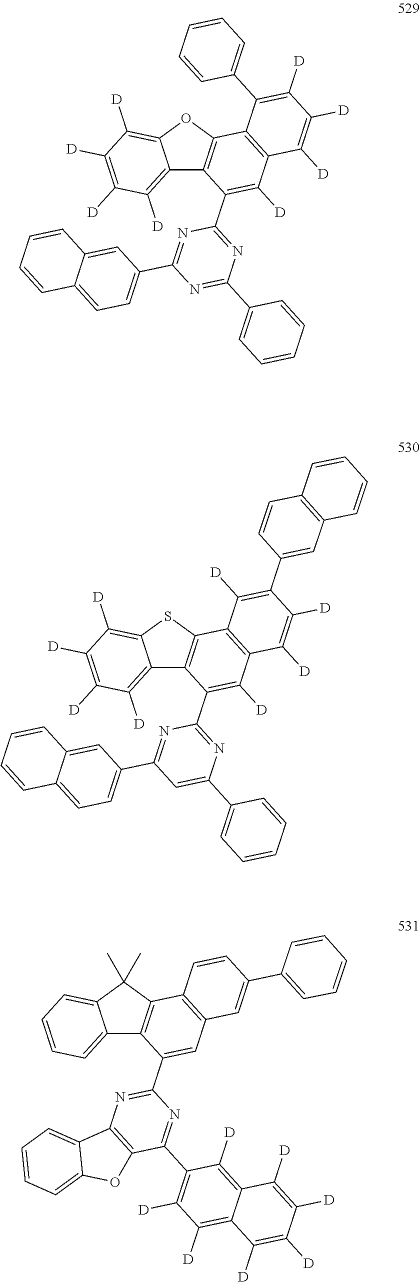 [SMILES: [2H]c1c([2H])c([2H])c2c(oc3c4c(-c5ccccc5)c([2H])c([2H])c([2H])c4c([2H])c(-c4nc(-c5ccccc5)nc(-c5ccc6ccccc6c5)n4)c32)c1[2H] ; [2H]c1c([2H])c([2H])c2c(sc3c4c([2H])c(-c5ccc6ccccc6c5)c([2H])c([2H])c4c([2H])c(-c4nc(-c5ccccc5)cc(-c5ccc6ccccc6c5)n4)c32)c1[2H] ; [2H]c1c([2H])c([2H])c2c([2H])c(-c3nc(-c4cc5cc(-c6ccccc6)ccc5c5c4-c4ccccc4C5(C)C)nc4c3oc3ccccc34)c([2H])c([2H])c2c1[2H]]

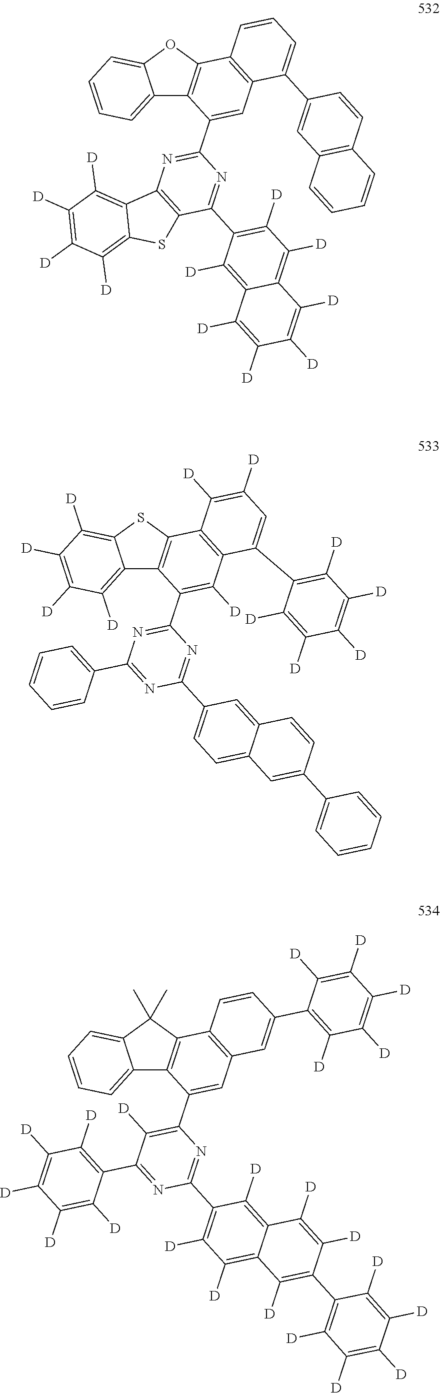

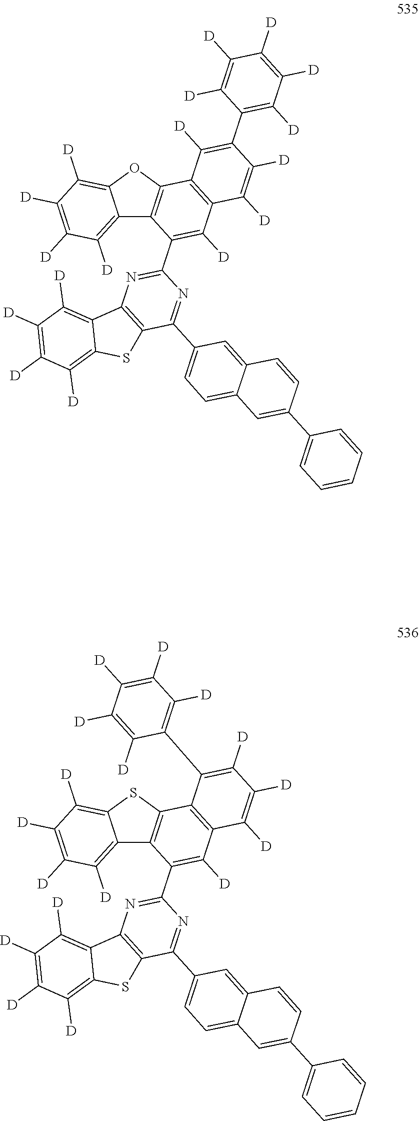 [SMILES: [2H]c1c([2H])c([2H])c(-c2c([2H])c([2H])c3c([2H])c(-c4nc(-c5ccc6cc(-c7ccccc7)ccc6c5)c5sc6c([2H])c([2H])c([2H])c([2H])c6c5n4)c4c(oc5c([2H])c([2H])c([2H])c([2H])c54)c3c2[2H])c([2H])c1[2H] ; [2H]c1c([2H])c([2H])c(-c2c([2H])c([2H])c([2H])c3c([2H])c(-c4nc(-c5ccc6cc(-c7ccccc7)ccc6c5)c5sc6c([2H])c([2H])c([2H])c([2H])c6c5n4)c4c(sc5c([2H])c([2H])c([2H])c([2H])c54)c23)c([2H])c1[2H]]

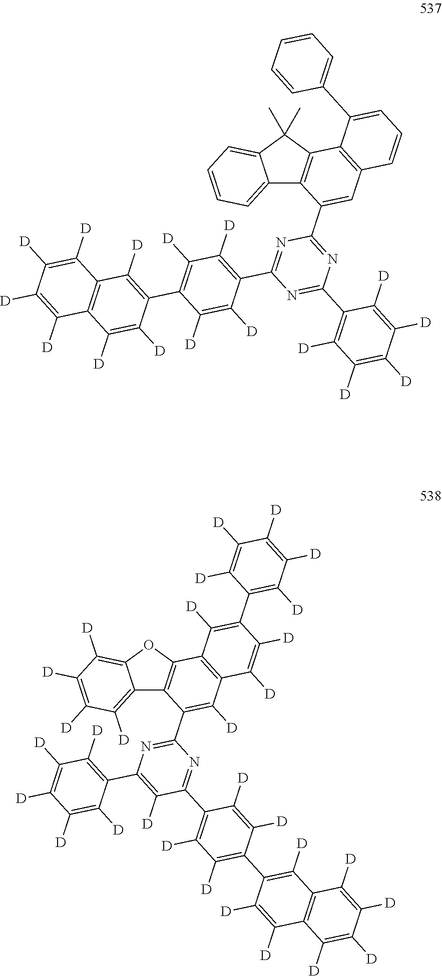 [SMILES: [2H]c1c([2H])c([2H])c(-c2nc(-c3cc4cccc(-c5ccccc5)c4c4c3-c3ccccc3C4(C)C)nc(-c3c([2H])c([2H])c(-c4c([2H])c([2H])c5c([2H])c([2H])c([2H])c([2H])c5c4[2H])c([2H])c3[2H])n2)c([2H])c1[2H] ; [2H]c1c(-c2c([2H])c([2H])c([2H])c([2H])c2[2H])nc(-c2c([2H])c3c([2H])c([2H])c(-c4c([2H])c([2H])c([2H])c([2H])c4[2H])c([2H])c3c3oc4c([2H])c([2H])c([2H])c([2H])c4c23)nc1-c1c([2H])c([2H])c(-c2c([2H])c([2H])c3c([2H])c([2H])c([2H])c([2H])c3c2[2H])c([2H])c1[2H]]

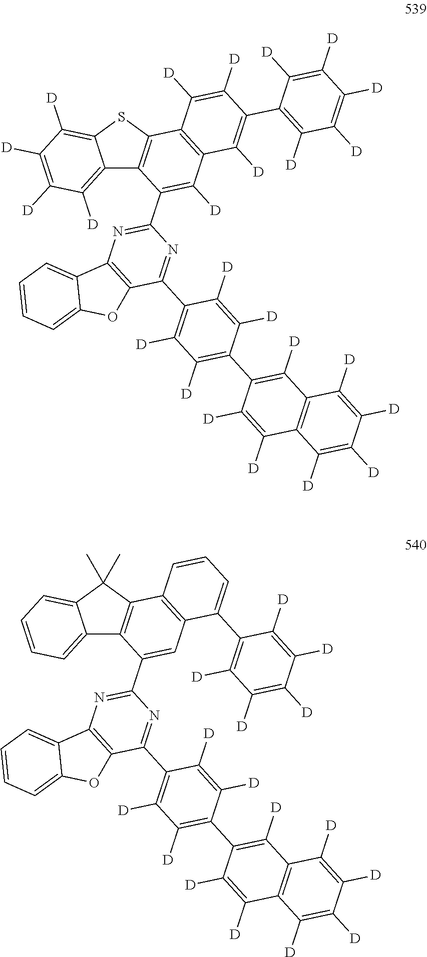 [SMILES: [2H]c1c([2H])c([2H])c(-c2c([2H])c([2H])c3c(c2[2H])c([2H])c(-c2nc(-c4c([2H])c([2H])c(-c5c([2H])c([2H])c6c([2H])c([2H])c([2H])c([2H])c6c5[2H])c([2H])c4[2H])c4oc5ccccc5c4n2)c2c3sc3c([2H])c([2H])c([2H])c([2H])c32)c([2H])c1[2H] ; [2H]c1c([2H])c([2H])c(-c2cccc3c4c(c(-c5nc(-c6c([2H])c([2H])c(-c7c([2H])c([2H])c8c([2H])c([2H])c([2H])c([2H])c8c7[2H])c([2H])c6[2H])c6oc7ccccc7c6n5)cc23)-c2ccccc2C4(C)C)c([2H])c1[2H]]

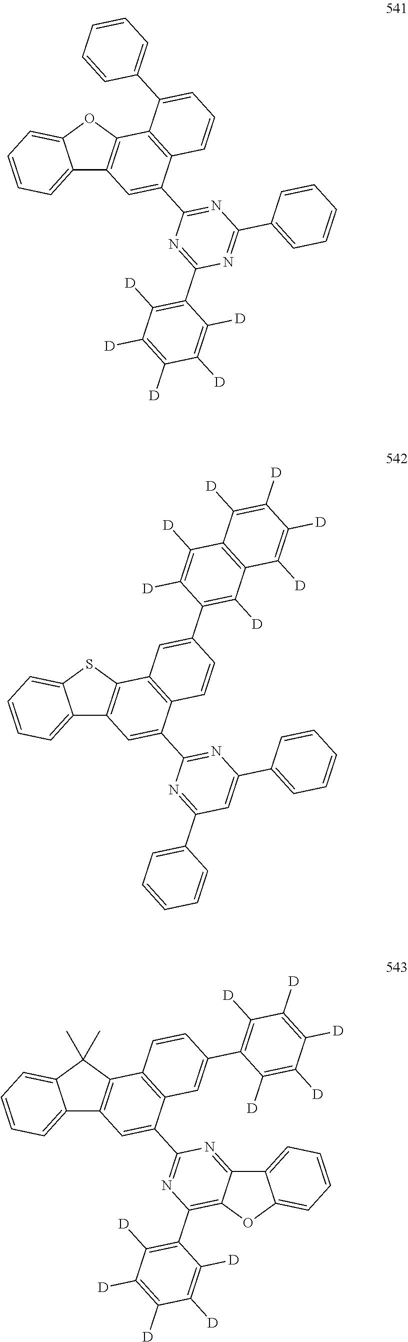 [SMILES: [2H]c1c([2H])c([2H])c(-c2nc(-c3ccccc3)nc(-c3cc4c5ccccc5oc4c4c(-c5ccccc5)cccc34)n2)c([2H])c1[2H] ; [2H]c1c([2H])c([2H])c2c([2H])c(-c3ccc4c(-c5nc(-c6ccccc6)cc(-c6ccccc6)n5)cc5c6ccccc6sc5c4c3)c([2H])c([2H])c2c1[2H] ; [2H]c1c([2H])c([2H])c(-c2ccc3c4c(cc(-c5nc(-c6c([2H])c([2H])c([2H])c([2H])c6[2H])c6oc7ccccc7c6n5)c3c2)-c2ccccc2C4(C)C)c([2H])c1[2H]]

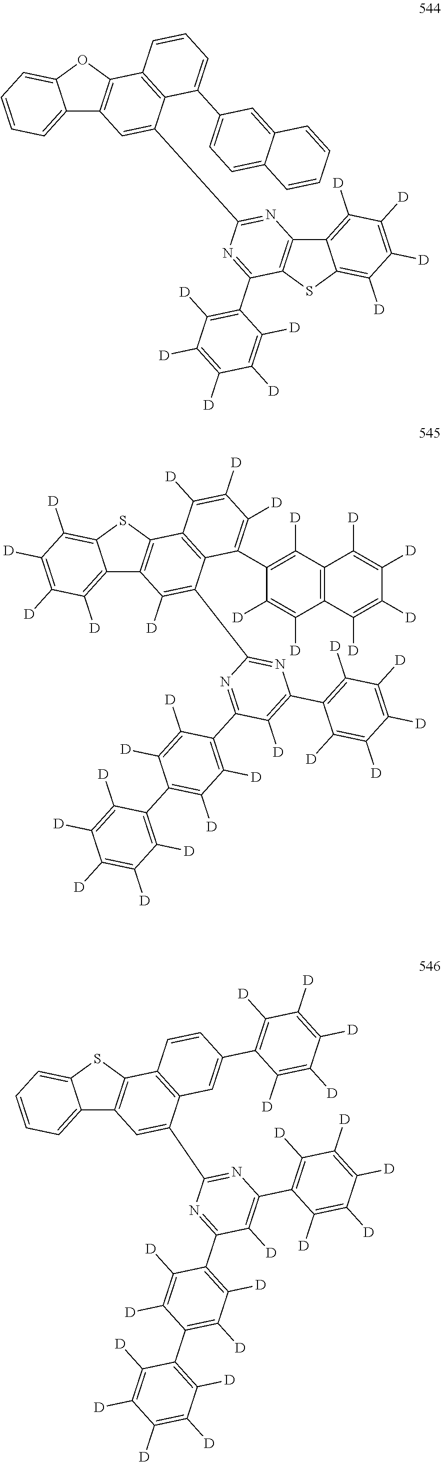

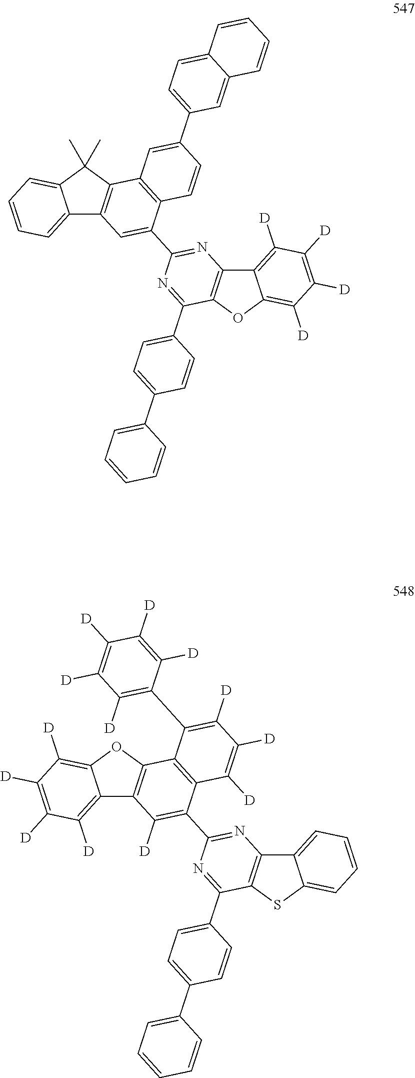

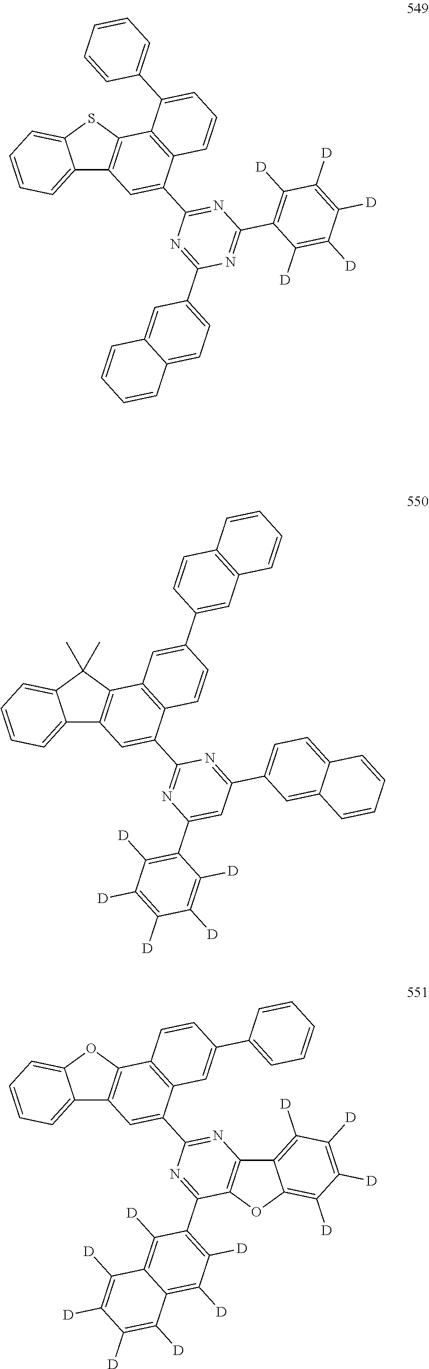 [SMILES: [2H]c1c([2H])c([2H])c(-c2nc(-c3ccc4ccccc4c3)nc(-c3cc4c5ccccc5sc4c4c(-c5ccccc5)cccc34)n2)c([2H])c1[2H] ; [2H]c1c([2H])c([2H])c(-c2cc(-c3ccc4ccccc4c3)nc(-c3cc4c(c5cc(-c6ccc7ccccc7c6)ccc35)C(C)(C)c3ccccc3-4)n2)c([2H])c1[2H] ; [2H]c1c([2H])c([2H])c2c(oc3c(-c4c([2H])c([2H])c5c([2H])c([2H])c([2H])c([2H])c5c4[2H])nc(-c4cc5c6ccccc6oc5c5ccc(-c6ccccc6)cc45)nc32)c1[2H]]

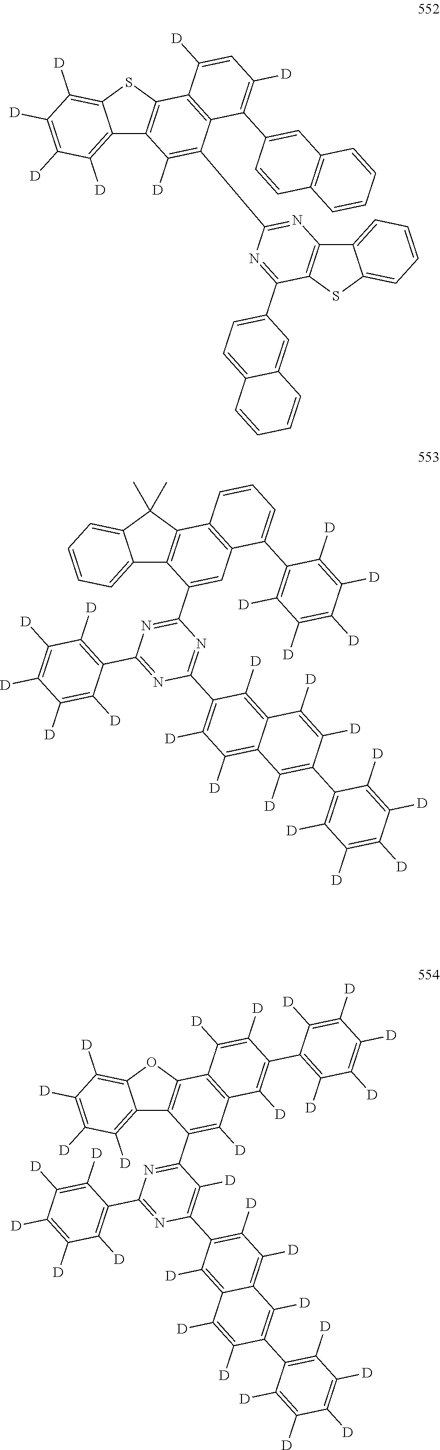

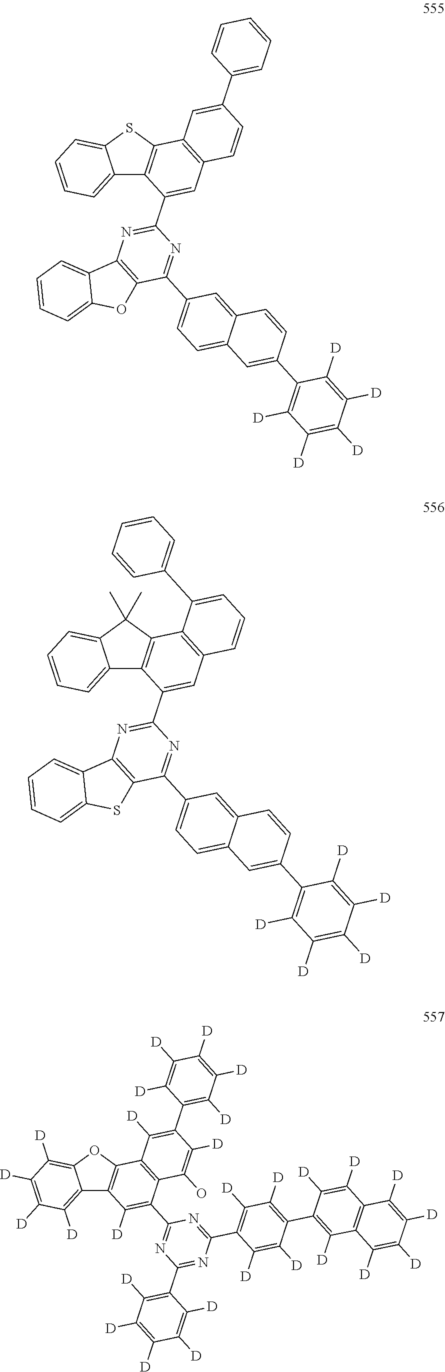

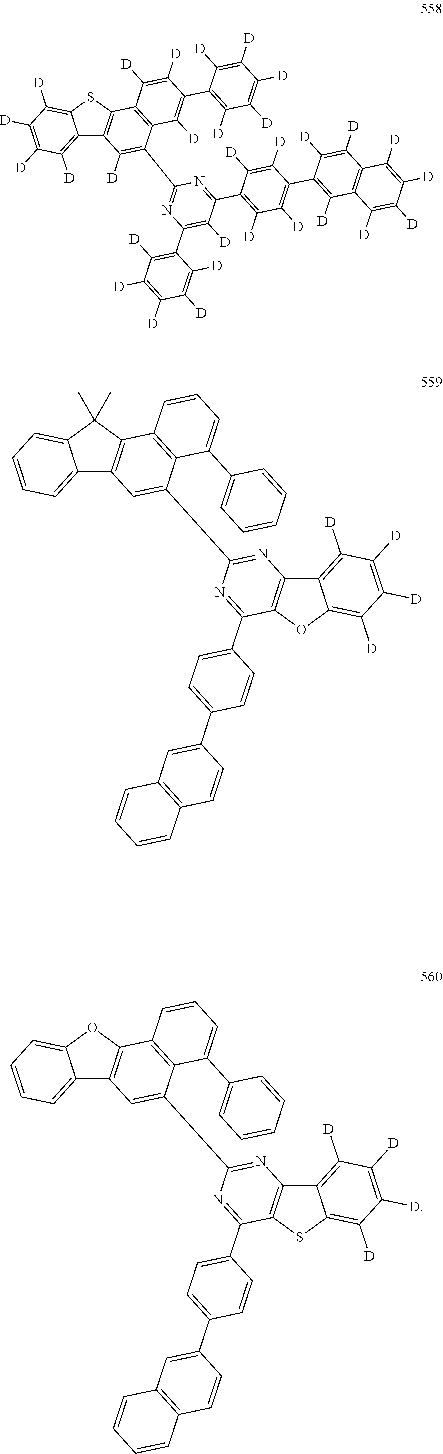

8. An organic light emitting device comprising: a first electrode; a second electrode provided to face the first electrode; and an organic material layer having one or more layers provided between the first electrode and the second electrode, wherein the one or more layers of the organic material layer comprise the heterocyclic compound of claim 1.

9. The organic light emitting device of claim 8, wherein the organic material layer further comprises a heterocyclic compound represented by the following Chemical Formula 2: 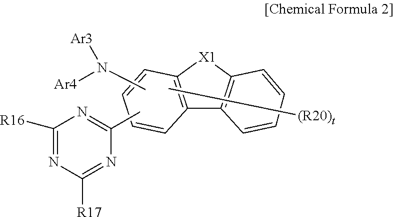
in Chemical Formula 2,
X1 is O or S,
R20 is hydrogen; deuterium; a halogen; a cyano group; a substituted or unsubstituted C1 to C60 alkyl group; a substituted or unsubstituted C6 to C60 aryl group; or a substituted or unsubstituted C2 to C60 heteroaryl group,
t is an integer from 1 to 4, and when t is 2 or higher, substituents in the parenthesis are the same as or different from each other,
Ar3 and Ar4 are the same as or different from each other, and are each independently a substituted or unsubstituted C6 to C60 aryl group; or a substituted or unsubstituted C2 to C60 heteroaryl group, and
R16 and R17 are the same as or different from each other, and are each independently a substituted or unsubstituted C6 to C60 aryl group; or a substituted or unsubstituted C2 to C60 heteroaryl group.

10. The organic light emitting device of claim 9, wherein a deuterium content of the heterocyclic compound represented by Chemical Formula 2 is 0% to 100%.

11. The organic light emitting device of claim 9, wherein Chemical Formula 2 is represented by any one of the following compounds:

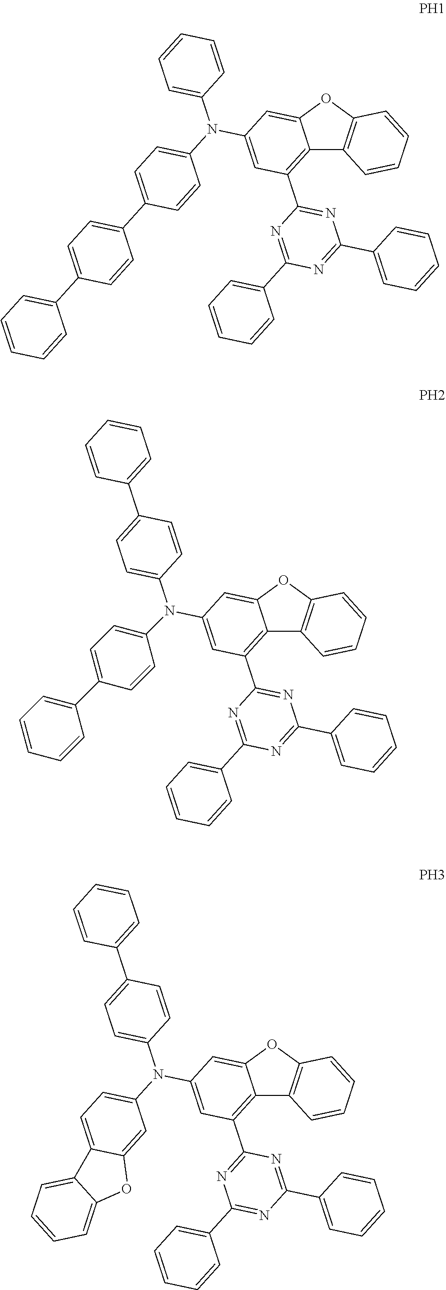 [SMILES: c1ccc(-c2ccc(-c3ccc(N(c4ccccc4)c4cc(-c5nc(-c6ccccc6)nc(-c6ccccc6)n5)c5c(c4)oc4ccccc45)cc3)cc2)cc1 ; c1ccc(-c2ccc(N(c3ccc(-c4ccccc4)cc3)c3cc(-c4nc(-c5ccccc5)nc(-c5ccccc5)n4)c4c(c3)oc3ccccc34)cc2)cc1 ; c1ccc(-c2ccc(N(c3ccc4c(c3)oc3ccccc34)c3cc(-c4nc(-c5ccccc5)nc(-c5ccccc5)n4)c4c(c3)oc3ccccc34)cc2)cc1]

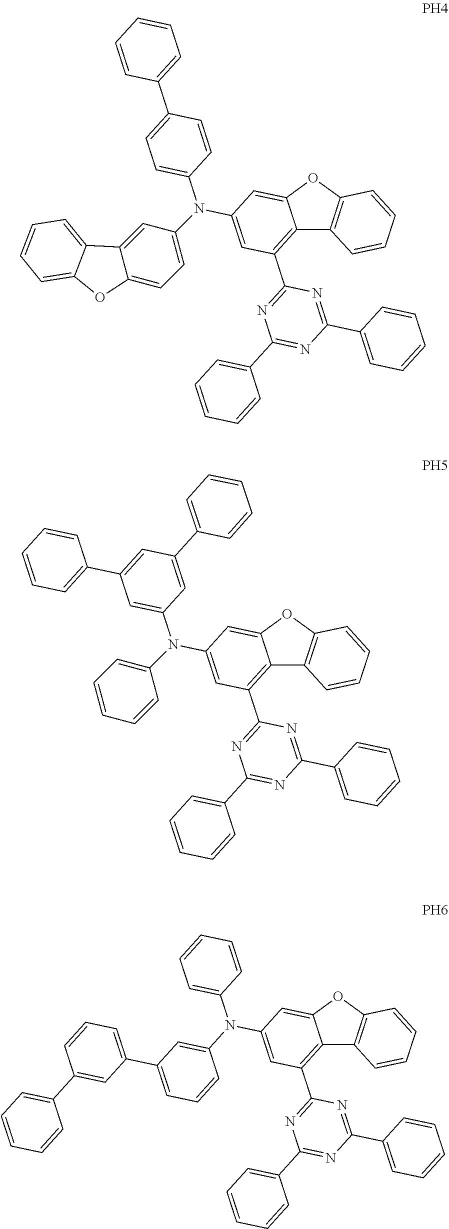 [SMILES: c1ccc(-c2ccc(N(c3cc(-c4nc(-c5ccccc5)nc(-c5ccccc5)n4)c4c(c3)oc3ccccc34)c3ccc4oc5ccccc5c4c3)cc2)cc1 ; c1ccc(-c2cc(-c3ccccc3)cc(N(c3ccccc3)c3cc(-c4nc(-c5ccccc5)nc(-c5ccccc5)n4)c4c(c3)oc3ccccc34)c2)cc1 ; c1ccc(-c2cccc(-c3cccc(N(c4ccccc4)c4cc(-c5nc(-c6ccccc6)nc(-c6ccccc6)n5)c5c(c4)oc4ccccc45)c3)c2)cc1]

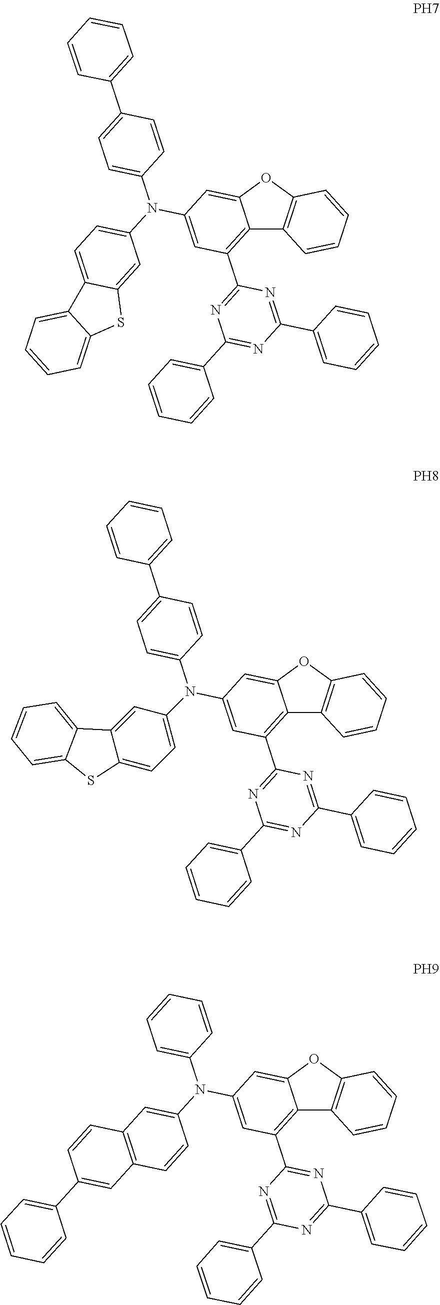 [SMILES: c1ccc(-c2ccc(N(c3cc(-c4nc(-c5ccccc5)nc(-c5ccccc5)n4)c4c(c3)oc3ccccc34)c3ccc4c(c3)sc3ccccc34)cc2)cc1 ; c1ccc(-c2ccc(N(c3cc(-c4nc(-c5ccccc5)nc(-c5ccccc5)n4)c4c(c3)oc3ccccc34)c3ccc4sc5ccccc5c4c3)cc2)cc1 ; c1ccc(-c2ccc3cc(N(c4ccccc4)c4cc(-c5nc(-c6ccccc6)nc(-c6ccccc6)n5)c5c(c4)oc4ccccc45)ccc3c2)cc1]

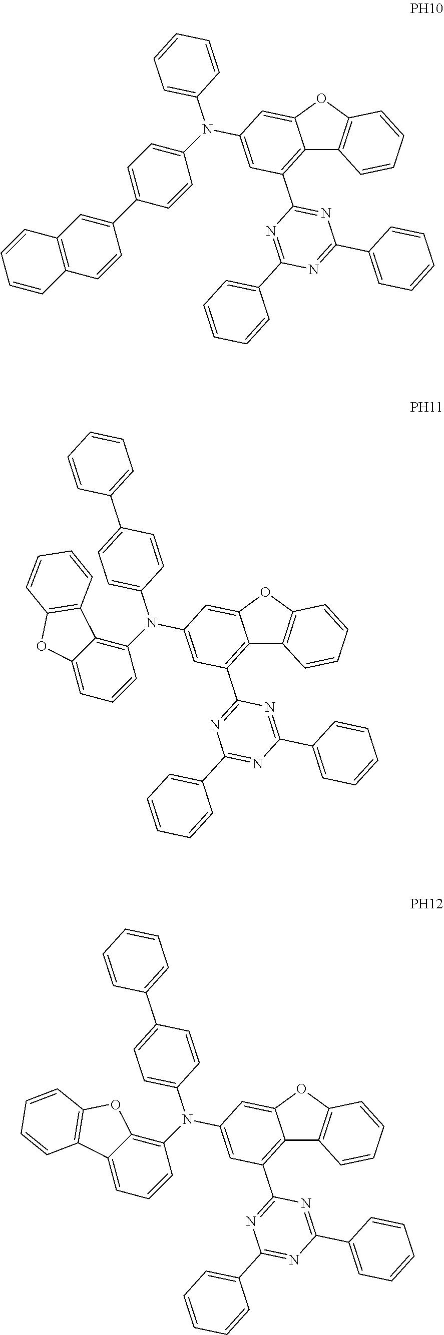 [SMILES: c1ccc(-c2nc(-c3ccccc3)nc(-c3cc(N(c4ccccc4)c4ccc(-c5ccc6ccccc6c5)cc4)cc4oc5ccccc5c34)n2)cc1 ; c1ccc(-c2ccc(N(c3cc(-c4nc(-c5ccccc5)nc(-c5ccccc5)n4)c4c(c3)oc3ccccc34)c3cccc4oc5ccccc5c34)cc2)cc1 ; c1ccc(-c2ccc(N(c3cc(-c4nc(-c5ccccc5)nc(-c5ccccc5)n4)c4c(c3)oc3ccccc34)c3cccc4c3oc3ccccc34)cc2)cc1]

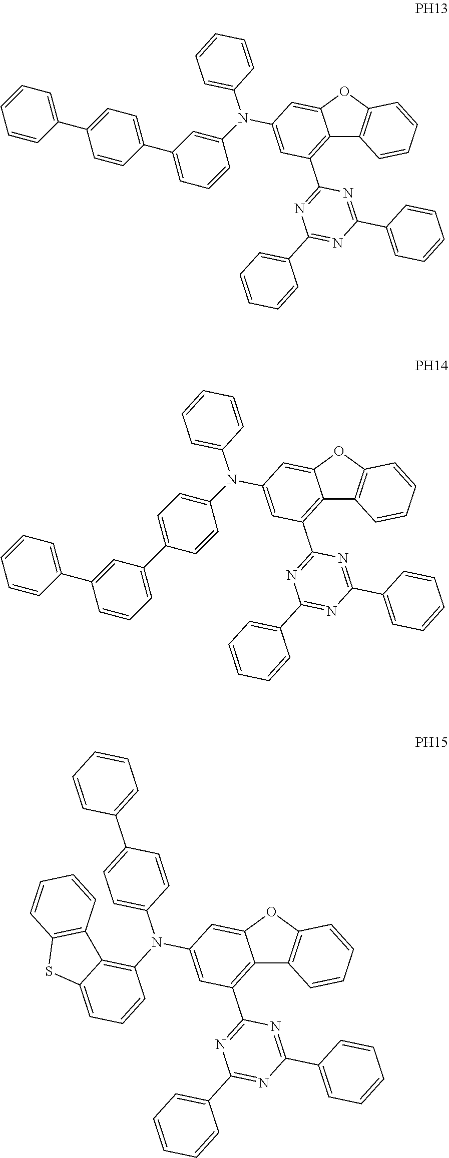 [SMILES: c1ccc(-c2ccc(-c3cccc(N(c4ccccc4)c4cc(-c5nc(-c6ccccc6)nc(-c6ccccc6)n5)c5c(c4)oc4ccccc45)c3)cc2)cc1 ; c1ccc(-c2cccc(-c3ccc(N(c4ccccc4)c4cc(-c5nc(-c6ccccc6)nc(-c6ccccc6)n5)c5c(c4)oc4ccccc45)cc3)c2)cc1 ; c1ccc(-c2ccc(N(c3cc(-c4nc(-c5ccccc5)nc(-c5ccccc5)n4)c4c(c3)oc3ccccc34)c3cccc4sc5ccccc5c34)cc2)cc1]

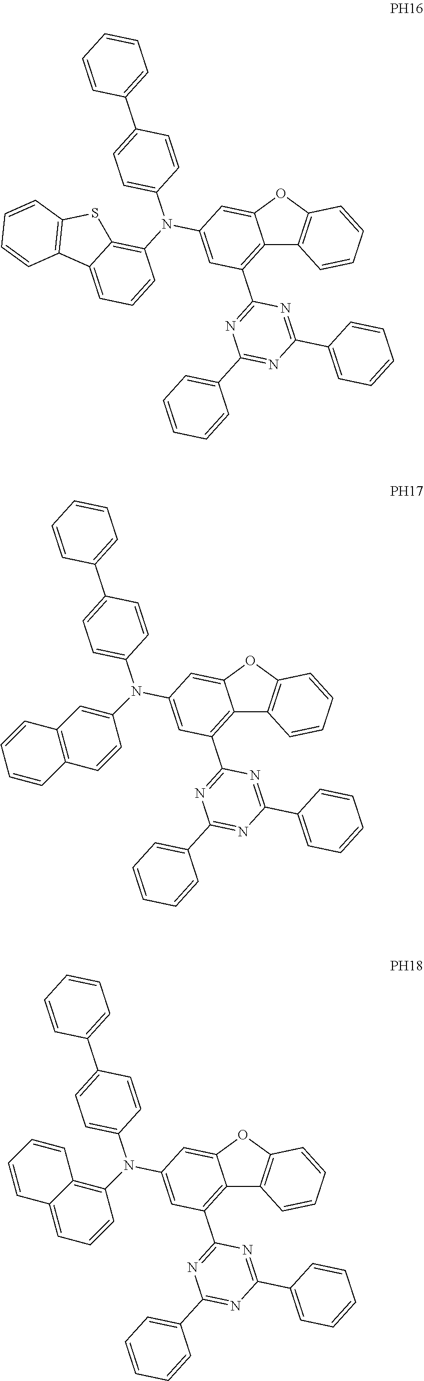 [SMILES: c1ccc(-c2ccc(N(c3cc(-c4nc(-c5ccccc5)nc(-c5ccccc5)n4)c4c(c3)oc3ccccc34)c3cccc4c3sc3ccccc34)cc2)cc1 ; c1ccc(-c2ccc(N(c3ccc4ccccc4c3)c3cc(-c4nc(-c5ccccc5)nc(-c5ccccc5)n4)c4c(c3)oc3ccccc34)cc2)cc1 ; c1ccc(-c2ccc(N(c3cc(-c4nc(-c5ccccc5)nc(-c5ccccc5)n4)c4c(c3)oc3ccccc34)c3cccc4ccccc34)cc2)cc1]

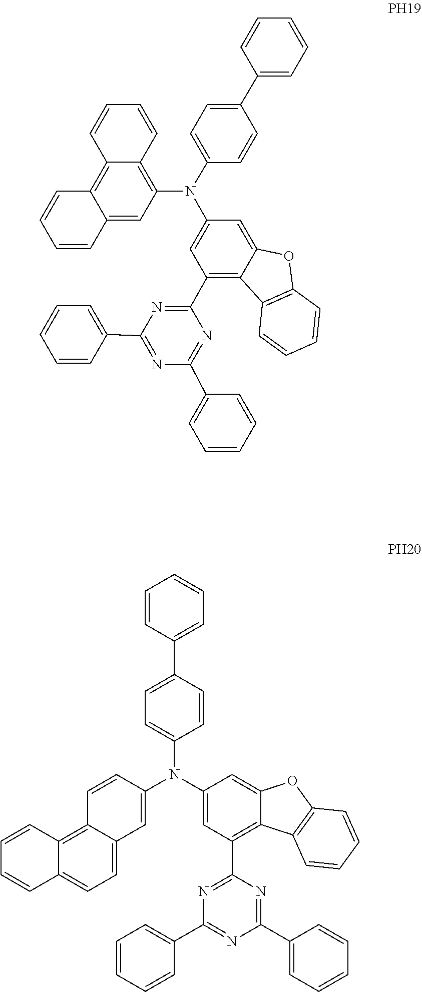 [SMILES: c1ccc(-c2ccc(N(c3cc(-c4nc(-c5ccccc5)nc(-c5ccccc5)n4)c4c(c3)oc3ccccc34)c3cc4ccccc4c4ccccc34)cc2)cc1 ; c1ccc(-c2ccc(N(c3ccc4c(ccc5ccccc54)c3)c3cc(-c4nc(-c5ccccc5)nc(-c5ccccc5)n4)c4c(c3)oc3ccccc34)cc2)cc1]

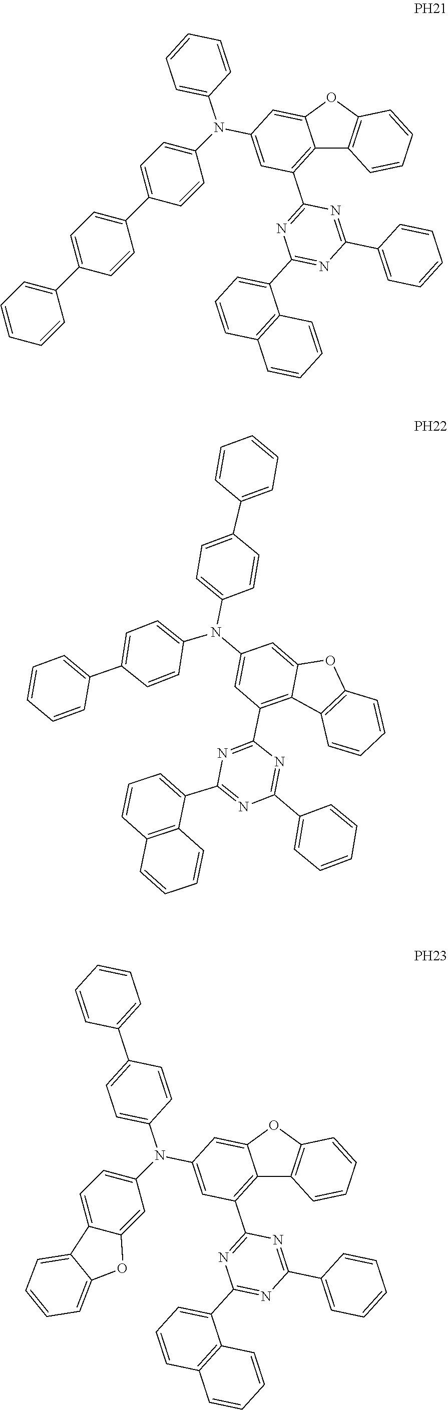 [SMILES: c1ccc(-c2ccc(-c3ccc(N(c4ccccc4)c4cc(-c5nc(-c6ccccc6)nc(-c6cccc7ccccc67)n5)c5c(c4)oc4ccccc45)cc3)cc2)cc1 ; c1ccc(-c2ccc(N(c3ccc(-c4ccccc4)cc3)c3cc(-c4nc(-c5ccccc5)nc(-c5cccc6ccccc56)n4)c4c(c3)oc3ccccc34)cc2)cc1 ; c1ccc(-c2ccc(N(c3ccc4c(c3)oc3ccccc34)c3cc(-c4nc(-c5ccccc5)nc(-c5cccc6ccccc56)n4)c4c(c3)oc3ccccc34)cc2)cc1]

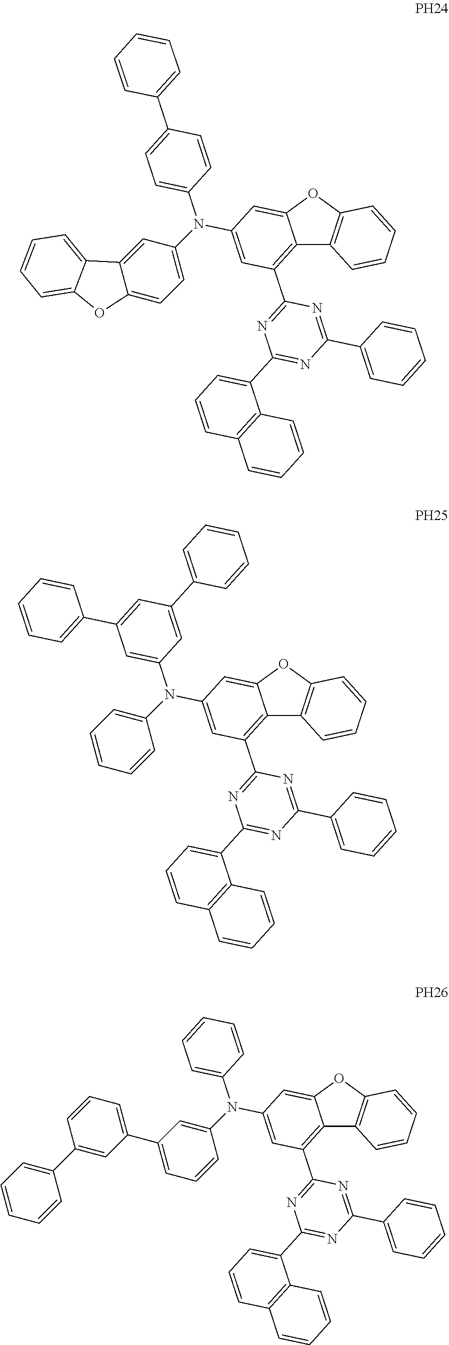 [SMILES: c1ccc(-c2ccc(N(c3cc(-c4nc(-c5ccccc5)nc(-c5cccc6ccccc56)n4)c4c(c3)oc3ccccc34)c3ccc4oc5ccccc5c4c3)cc2)cc1 ; c1ccc(-c2cc(-c3ccccc3)cc(N(c3ccccc3)c3cc(-c4nc(-c5ccccc5)nc(-c5cccc6ccccc56)n4)c4c(c3)oc3ccccc34)c2)cc1 ; c1ccc(-c2cccc(-c3cccc(N(c4ccccc4)c4cc(-c5nc(-c6ccccc6)nc(-c6cccc7ccccc67)n5)c5c(c4)oc4ccccc45)c3)c2)cc1]

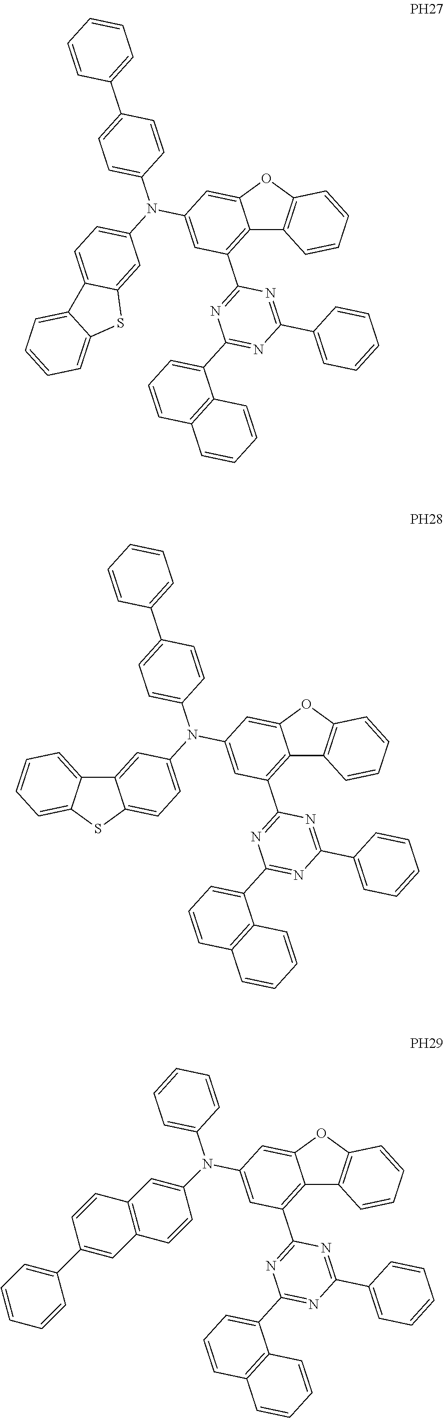 [SMILES: c1ccc(-c2ccc(N(c3cc(-c4nc(-c5ccccc5)nc(-c5cccc6ccccc56)n4)c4c(c3)oc3ccccc34)c3ccc4c(c3)sc3ccccc34)cc2)cc1 ; c1ccc(-c2ccc(N(c3cc(-c4nc(-c5ccccc5)nc(-c5cccc6ccccc56)n4)c4c(c3)oc3ccccc34)c3ccc4sc5ccccc5c4c3)cc2)cc1 ; c1ccc(-c2ccc3cc(N(c4ccccc4)c4cc(-c5nc(-c6ccccc6)nc(-c6cccc7ccccc67)n5)c5c(c4)oc4ccccc45)ccc3c2)cc1]

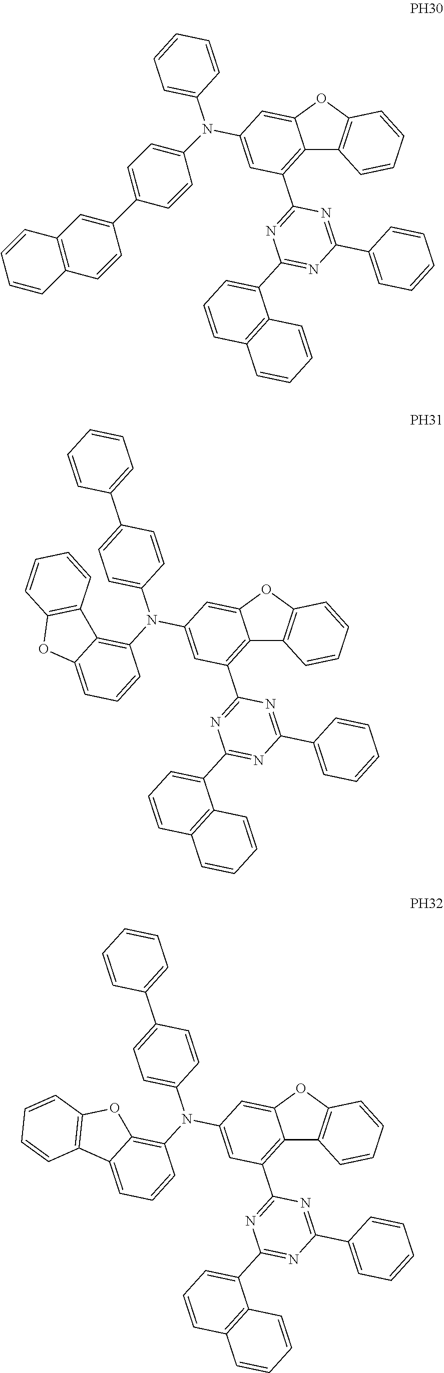 [SMILES: c1ccc(-c2nc(-c3cccc4ccccc34)nc(-c3cc(N(c4ccccc4)c4ccc(-c5ccc6ccccc6c5)cc4)cc4oc5ccccc5c34)n2)cc1 ; c1ccc(-c2ccc(N(c3cc(-c4nc(-c5ccccc5)nc(-c5cccc6ccccc56)n4)c4c(c3)oc3ccccc34)c3cccc4oc5ccccc5c34)cc2)cc1 ; c1ccc(-c2ccc(N(c3cc(-c4nc(-c5ccccc5)nc(-c5cccc6ccccc56)n4)c4c(c3)oc3ccccc34)c3cccc4c3oc3ccccc34)cc2)cc1]

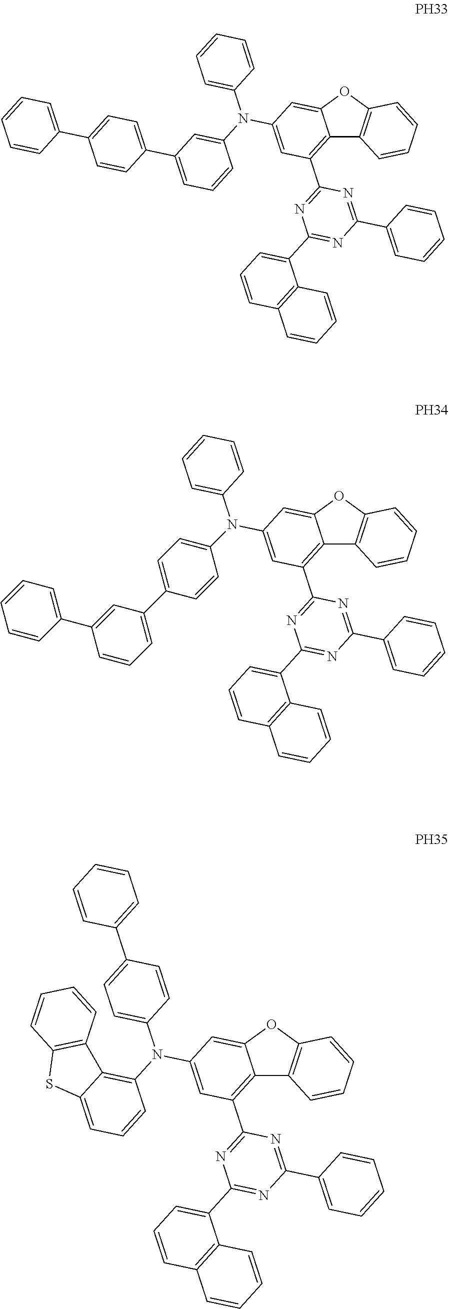 [SMILES: c1ccc(-c2ccc(-c3cccc(N(c4ccccc4)c4cc(-c5nc(-c6ccccc6)nc(-c6cccc7ccccc67)n5)c5c(c4)oc4ccccc45)c3)cc2)cc1 ; c1ccc(-c2cccc(-c3ccc(N(c4ccccc4)c4cc(-c5nc(-c6ccccc6)nc(-c6cccc7ccccc67)n5)c5c(c4)oc4ccccc45)cc3)c2)cc1 ; c1ccc(-c2ccc(N(c3cc(-c4nc(-c5ccccc5)nc(-c5cccc6ccccc56)n4)c4c(c3)oc3ccccc34)c3cccc4sc5ccccc5c34)cc2)cc1]

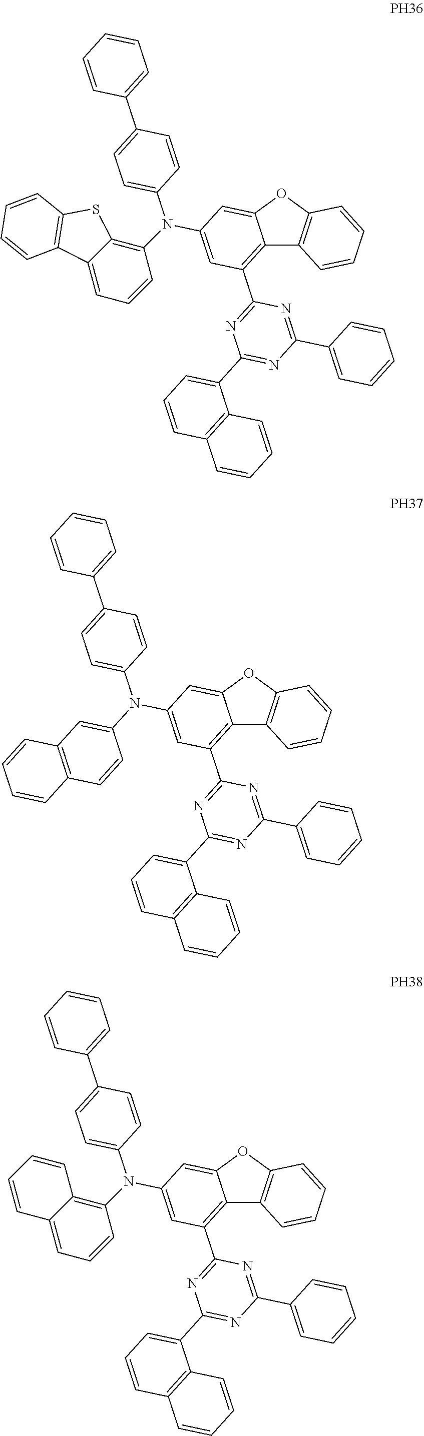 [SMILES: c1ccc(-c2ccc(N(c3cc(-c4nc(-c5ccccc5)nc(-c5cccc6ccccc56)n4)c4c(c3)oc3ccccc34)c3cccc4c3sc3ccccc34)cc2)cc1 ; c1ccc(-c2ccc(N(c3ccc4ccccc4c3)c3cc(-c4nc(-c5ccccc5)nc(-c5cccc6ccccc56)n4)c4c(c3)oc3ccccc34)cc2)cc1 ; c1ccc(-c2ccc(N(c3cc(-c4nc(-c5ccccc5)nc(-c5cccc6ccccc56)n4)c4c(c3)oc3ccccc34)c3cccc4ccccc34)cc2)cc1]

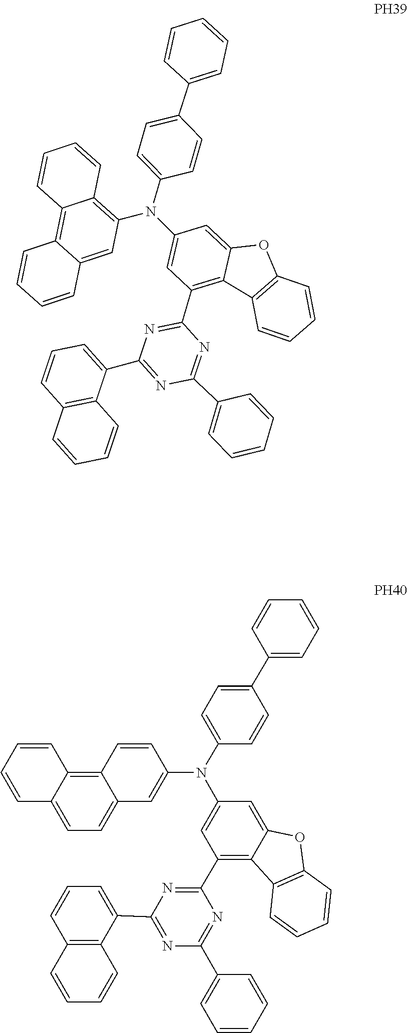 [SMILES: c1ccc(-c2ccc(N(c3cc(-c4nc(-c5ccccc5)nc(-c5cccc6ccccc56)n4)c4c(c3)oc3ccccc34)c3cc4ccccc4c4ccccc34)cc2)cc1 ; c1ccc(-c2ccc(N(c3ccc4c(ccc5ccccc54)c3)c3cc(-c4nc(-c5ccccc5)nc(-c5cccc6ccccc56)n4)c4c(c3)oc3ccccc34)cc2)cc1]

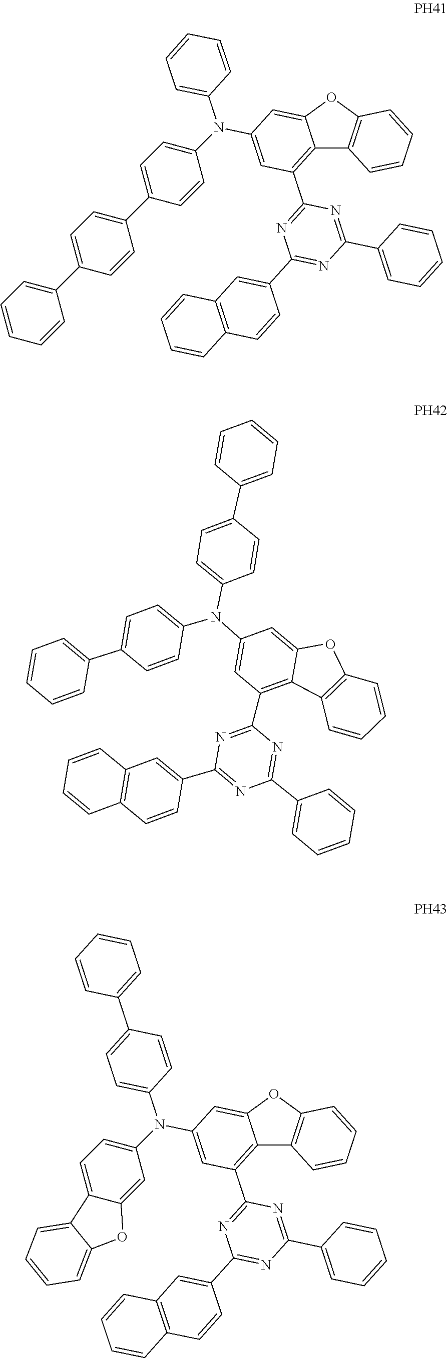 [SMILES: c1ccc(-c2ccc(-c3ccc(N(c4ccccc4)c4cc(-c5nc(-c6ccccc6)nc(-c6ccc7ccccc7c6)n5)c5c(c4)oc4ccccc45)cc3)cc2)cc1 ; c1ccc(-c2ccc(N(c3ccc(-c4ccccc4)cc3)c3cc(-c4nc(-c5ccccc5)nc(-c5ccc6ccccc6c5)n4)c4c(c3)oc3ccccc34)cc2)cc1 ; c1ccc(-c2ccc(N(c3ccc4c(c3)oc3ccccc34)c3cc(-c4nc(-c5ccccc5)nc(-c5ccc6ccccc6c5)n4)c4c(c3)oc3ccccc34)cc2)cc1]

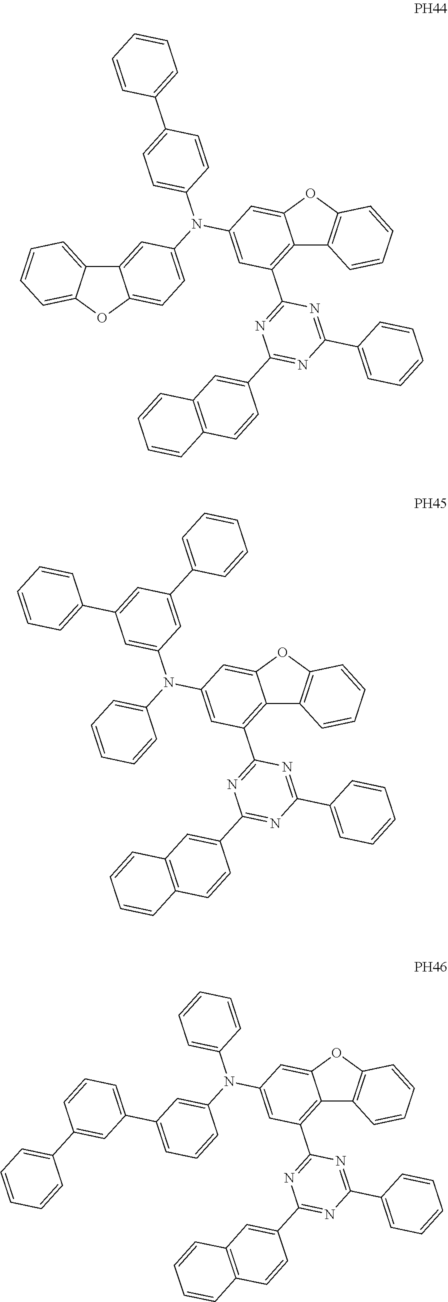 [SMILES: c1ccc(-c2ccc(N(c3cc(-c4nc(-c5ccccc5)nc(-c5ccc6ccccc6c5)n4)c4c(c3)oc3ccccc34)c3ccc4oc5ccccc5c4c3)cc2)cc1 ; c1ccc(-c2cc(-c3ccccc3)cc(N(c3ccccc3)c3cc(-c4nc(-c5ccccc5)nc(-c5ccc6ccccc6c5)n4)c4c(c3)oc3ccccc34)c2)cc1 ; c1ccc(-c2cccc(-c3cccc(N(c4ccccc4)c4cc(-c5nc(-c6ccccc6)nc(-c6ccc7ccccc7c6)n5)c5c(c4)oc4ccccc45)c3)c2)cc1]

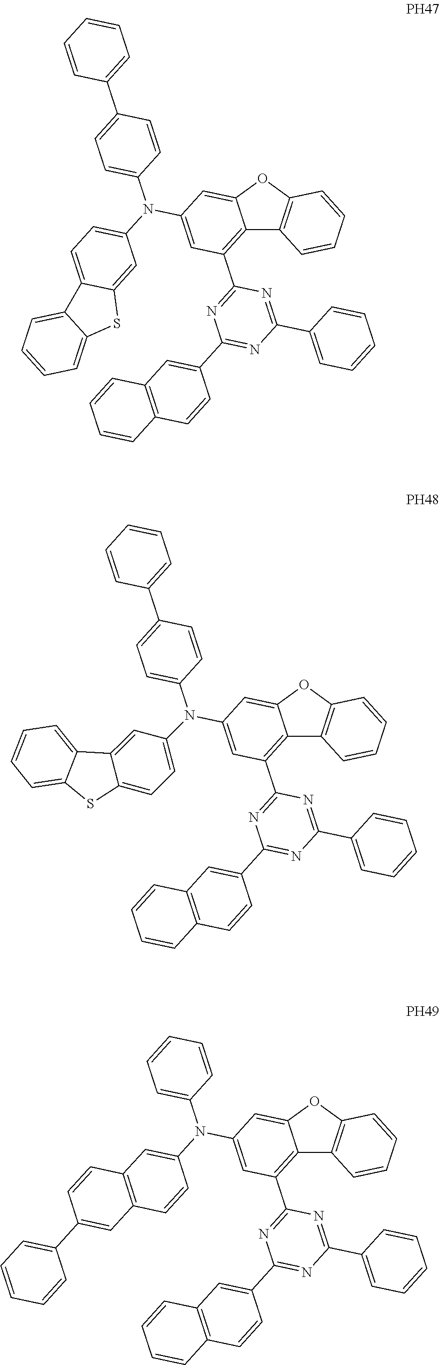 [SMILES: c1ccc(-c2ccc(N(c3cc(-c4nc(-c5ccccc5)nc(-c5ccc6ccccc6c5)n4)c4c(c3)oc3ccccc34)c3ccc4c(c3)sc3ccccc34)cc2)cc1 ; c1ccc(-c2ccc(N(c3cc(-c4nc(-c5ccccc5)nc(-c5ccc6ccccc6c5)n4)c4c(c3)oc3ccccc34)c3ccc4sc5ccccc5c4c3)cc2)cc1 ; c1ccc(-c2ccc3cc(N(c4ccccc4)c4cc(-c5nc(-c6ccccc6)nc(-c6ccc7ccccc7c6)n5)c5c(c4)oc4ccccc45)ccc3c2)cc1]

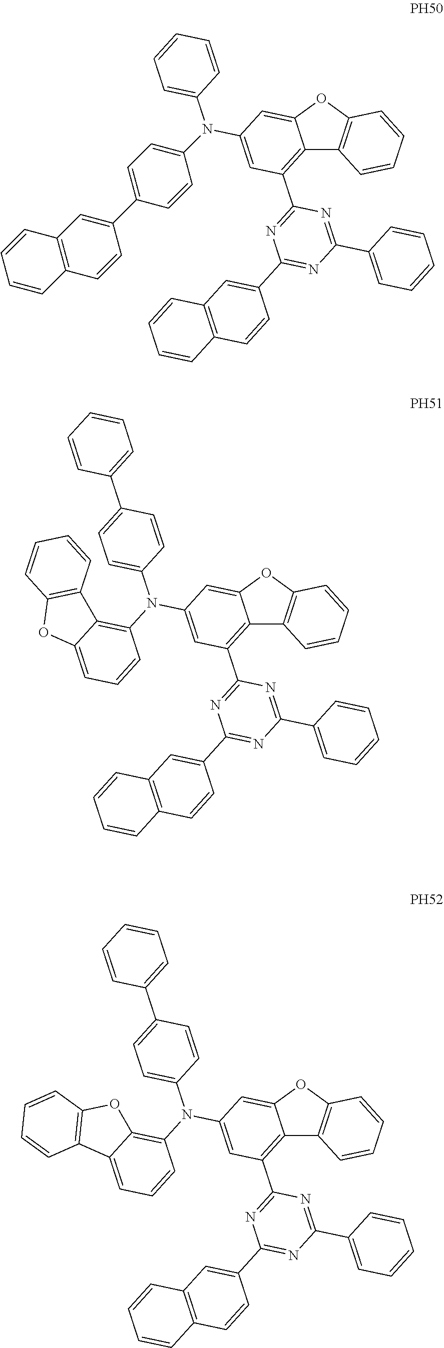 [SMILES: c1ccc(-c2nc(-c3ccc4ccccc4c3)nc(-c3cc(N(c4ccccc4)c4ccc(-c5ccc6ccccc6c5)cc4)cc4oc5ccccc5c34)n2)cc1 ; c1ccc(-c2ccc(N(c3cc(-c4nc(-c5ccccc5)nc(-c5ccc6ccccc6c5)n4)c4c(c3)oc3ccccc34)c3cccc4oc5ccccc5c34)cc2)cc1 ; c1ccc(-c2ccc(N(c3cc(-c4nc(-c5ccccc5)nc(-c5ccc6ccccc6c5)n4)c4c(c3)oc3ccccc34)c3cccc4c3oc3ccccc34)cc2)cc1]

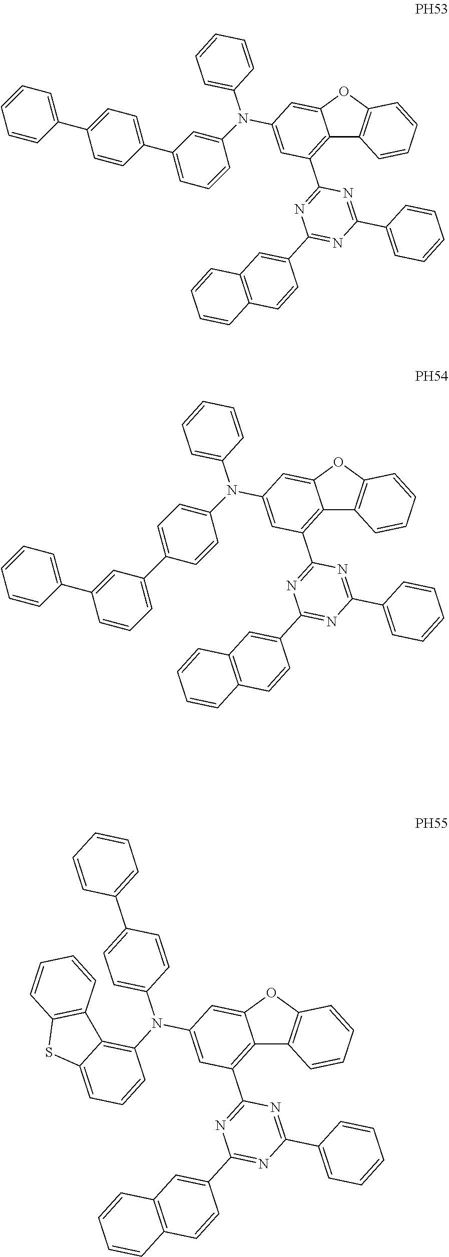 [SMILES: c1ccc(-c2ccc(-c3cccc(N(c4ccccc4)c4cc(-c5nc(-c6ccccc6)nc(-c6ccc7ccccc7c6)n5)c5c(c4)oc4ccccc45)c3)cc2)cc1 ; c1ccc(-c2cccc(-c3ccc(N(c4ccccc4)c4cc(-c5nc(-c6ccccc6)nc(-c6ccc7ccccc7c6)n5)c5c(c4)oc4ccccc45)cc3)c2)cc1 ; c1ccc(-c2ccc(N(c3cc(-c4nc(-c5ccccc5)nc(-c5ccc6ccccc6c5)n4)c4c(c3)oc3ccccc34)c3cccc4sc5ccccc5c34)cc2)cc1]

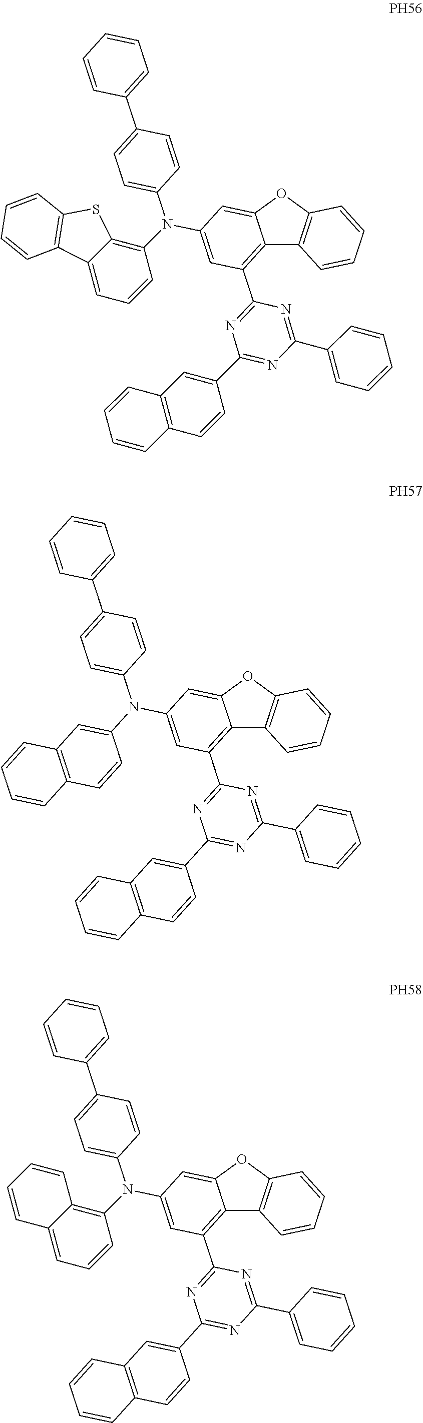 [SMILES: c1ccc(-c2ccc(N(c3cc(-c4nc(-c5ccccc5)nc(-c5ccc6ccccc6c5)n4)c4c(c3)oc3ccccc34)c3cccc4c3sc3ccccc34)cc2)cc1 ; c1ccc(-c2ccc(N(c3ccc4ccccc4c3)c3cc(-c4nc(-c5ccccc5)nc(-c5ccc6ccccc6c5)n4)c4c(c3)oc3ccccc34)cc2)cc1 ; c1ccc(-c2ccc(N(c3cc(-c4nc(-c5ccccc5)nc(-c5ccc6ccccc6c5)n4)c4c(c3)oc3ccccc34)c3cccc4ccccc34)cc2)cc1]

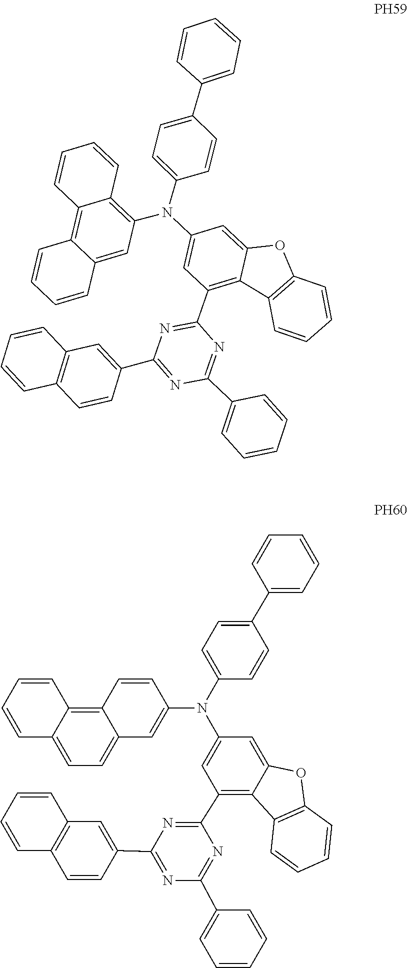 [SMILES: c1ccc(-c2ccc(N(c3cc(-c4nc(-c5ccccc5)nc(-c5ccc6ccccc6c5)n4)c4c(c3)oc3ccccc34)c3cc4ccccc4c4ccccc34)cc2)cc1 ; c1ccc(-c2ccc(N(c3ccc4c(ccc5ccccc54)c3)c3cc(-c4nc(-c5ccccc5)nc(-c5ccc6ccccc6c5)n4)c4c(c3)oc3ccccc34)cc2)cc1]

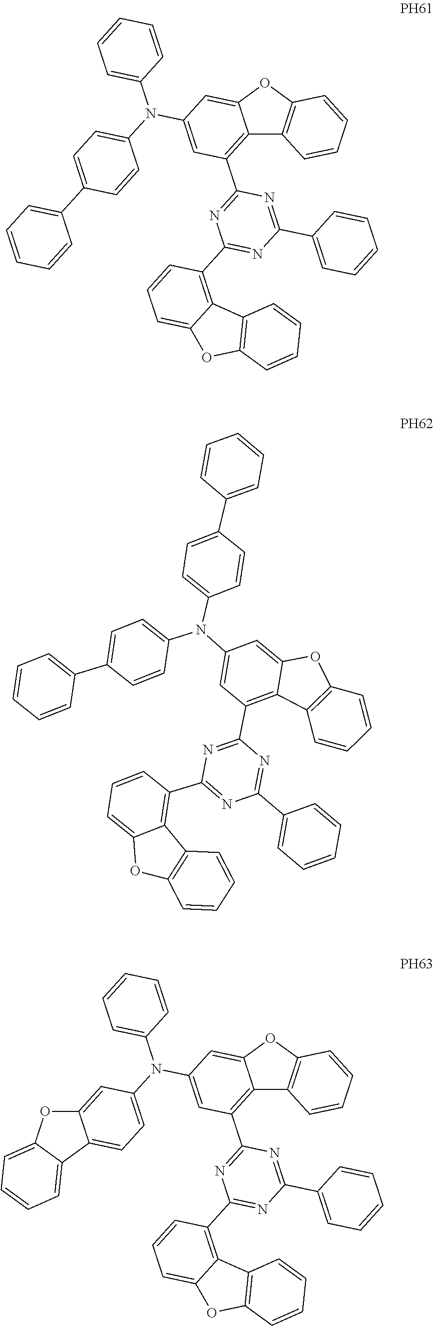 [SMILES: c1ccc(-c2ccc(N(c3ccccc3)c3cc(-c4nc(-c5ccccc5)nc(-c5cccc6oc7ccccc7c56)n4)c4c(c3)oc3ccccc34)cc2)cc1 ; c1ccc(-c2ccc(N(c3ccc(-c4ccccc4)cc3)c3cc(-c4nc(-c5ccccc5)nc(-c5cccc6oc7ccccc7c56)n4)c4c(c3)oc3ccccc34)cc2)cc1 ; c1ccc(-c2nc(-c3cccc4oc5ccccc5c34)nc(-c3cc(N(c4ccccc4)c4ccc5c(c4)oc4ccccc45)cc4oc5ccccc5c34)n2)cc1]

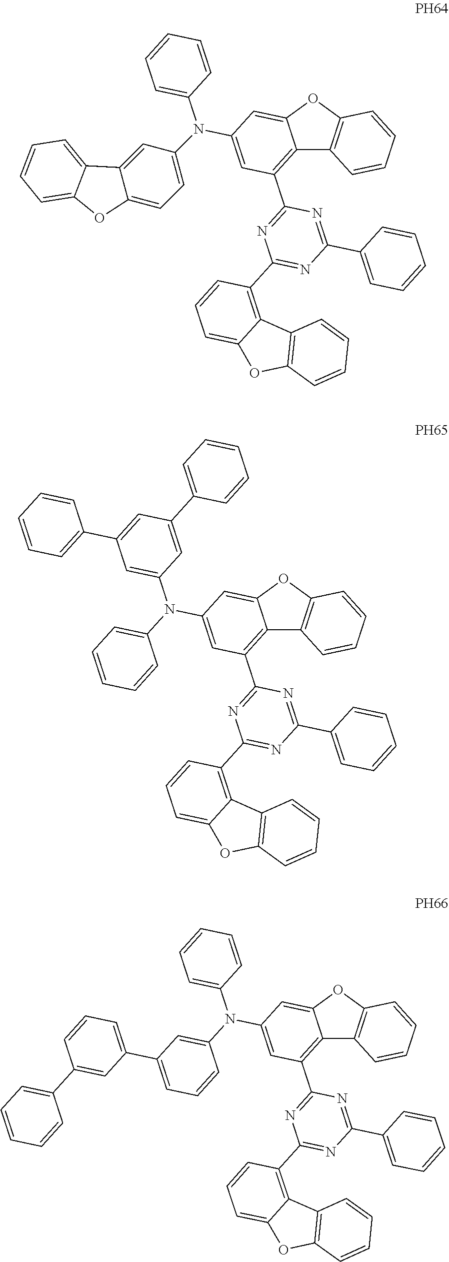 [SMILES: c1ccc(-c2nc(-c3cccc4oc5ccccc5c34)nc(-c3cc(N(c4ccccc4)c4ccc5oc6ccccc6c5c4)cc4oc5ccccc5c34)n2)cc1 ; c1ccc(-c2cc(-c3ccccc3)cc(N(c3ccccc3)c3cc(-c4nc(-c5ccccc5)nc(-c5cccc6oc7ccccc7c56)n4)c4c(c3)oc3ccccc34)c2)cc1 ; c1ccc(-c2cccc(-c3cccc(N(c4ccccc4)c4cc(-c5nc(-c6ccccc6)nc(-c6cccc7oc8ccccc8c67)n5)c5c(c4)oc4ccccc45)c3)c2)cc1]

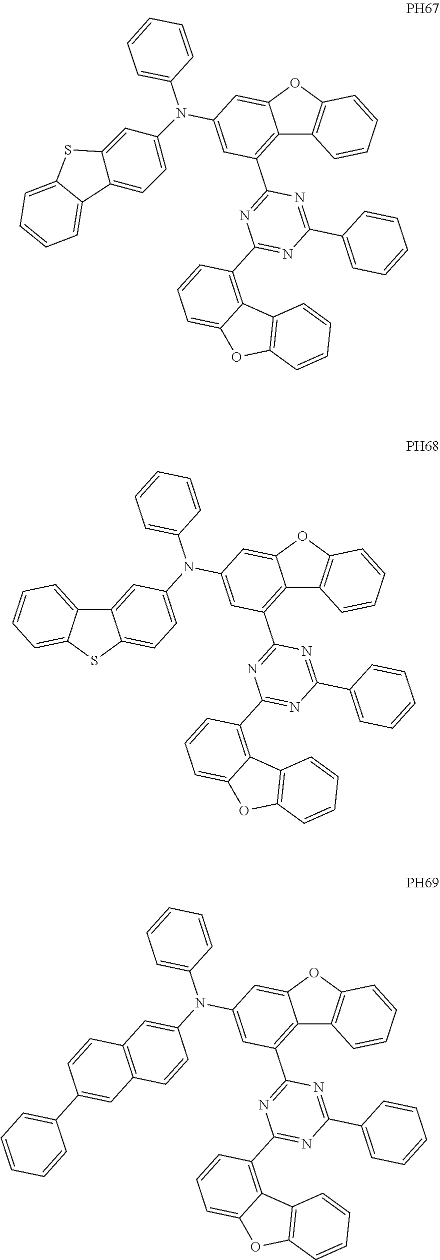 [SMILES: c1ccc(-c2nc(-c3cccc4oc5ccccc5c34)nc(-c3cc(N(c4ccccc4)c4ccc5c(c4)sc4ccccc45)cc4oc5ccccc5c34)n2)cc1 ; c1ccc(-c2nc(-c3cccc4oc5ccccc5c34)nc(-c3cc(N(c4ccccc4)c4ccc5sc6ccccc6c5c4)cc4oc5ccccc5c34)n2)cc1 ; c1ccc(-c2ccc3cc(N(c4ccccc4)c4cc(-c5nc(-c6ccccc6)nc(-c6cccc7oc8ccccc8c67)n5)c5c(c4)oc4ccccc45)ccc3c2)cc1]

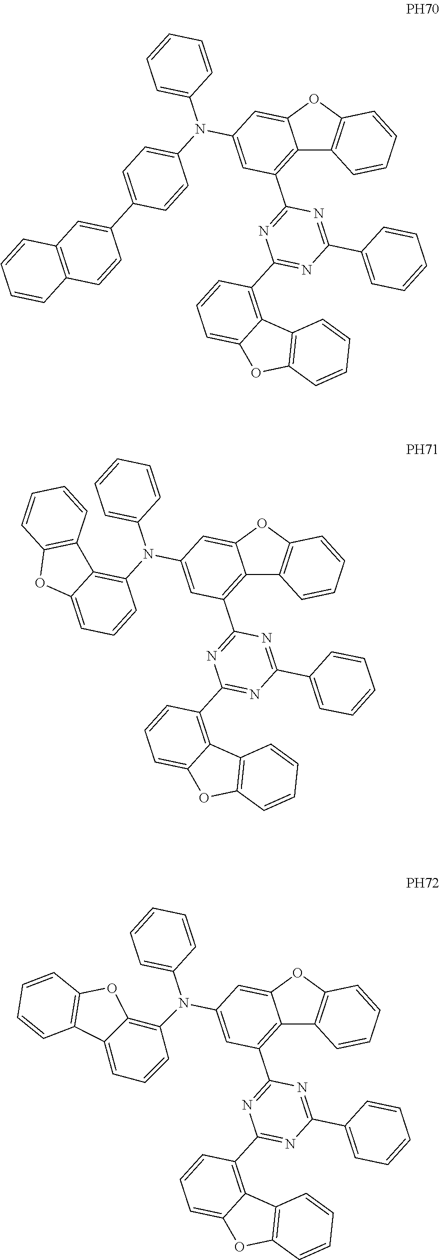 [SMILES: c1ccc(-c2nc(-c3cccc4oc5ccccc5c34)nc(-c3cc(N(c4ccccc4)c4ccc(-c5ccc6ccccc6c5)cc4)cc4oc5ccccc5c34)n2)cc1 ; c1ccc(-c2nc(-c3cccc4oc5ccccc5c34)nc(-c3cc(N(c4ccccc4)c4cccc5oc6ccccc6c45)cc4oc5ccccc5c34)n2)cc1 ; c1ccc(-c2nc(-c3cccc4oc5ccccc5c34)nc(-c3cc(N(c4ccccc4)c4cccc5c4oc4ccccc45)cc4oc5ccccc5c34)n2)cc1]

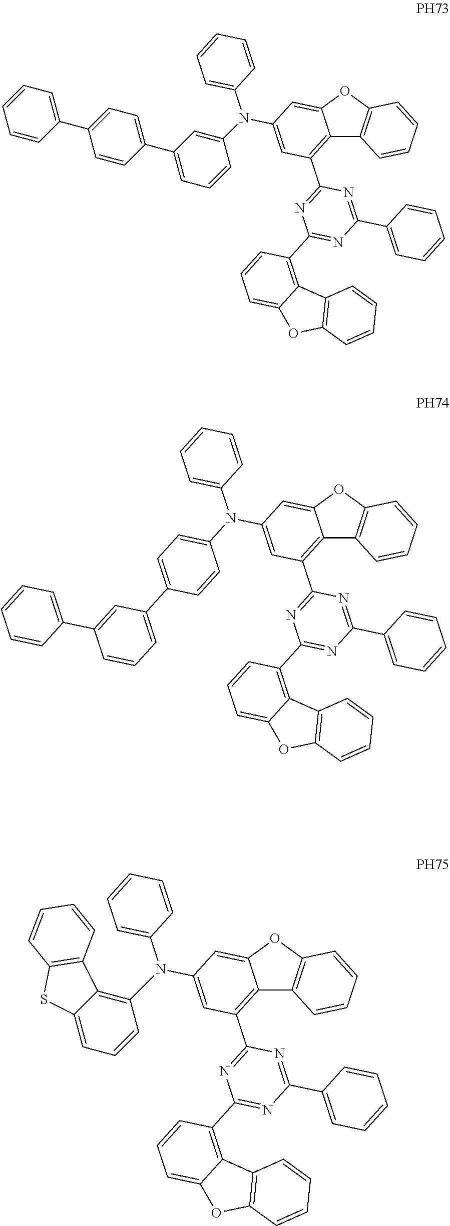 [SMILES: c1ccc(-c2ccc(-c3cccc(N(c4ccccc4)c4cc(-c5nc(-c6ccccc6)nc(-c6cccc7oc8ccccc8c67)n5)c5c(c4)oc4ccccc45)c3)cc2)cc1 ; c1ccc(-c2cccc(-c3ccc(N(c4ccccc4)c4cc(-c5nc(-c6ccccc6)nc(-c6cccc7oc8ccccc8c67)n5)c5c(c4)oc4ccccc45)cc3)c2)cc1 ; c1ccc(-c2nc(-c3cccc4oc5ccccc5c34)nc(-c3cc(N(c4ccccc4)c4cccc5sc6ccccc6c45)cc4oc5ccccc5c34)n2)cc1]

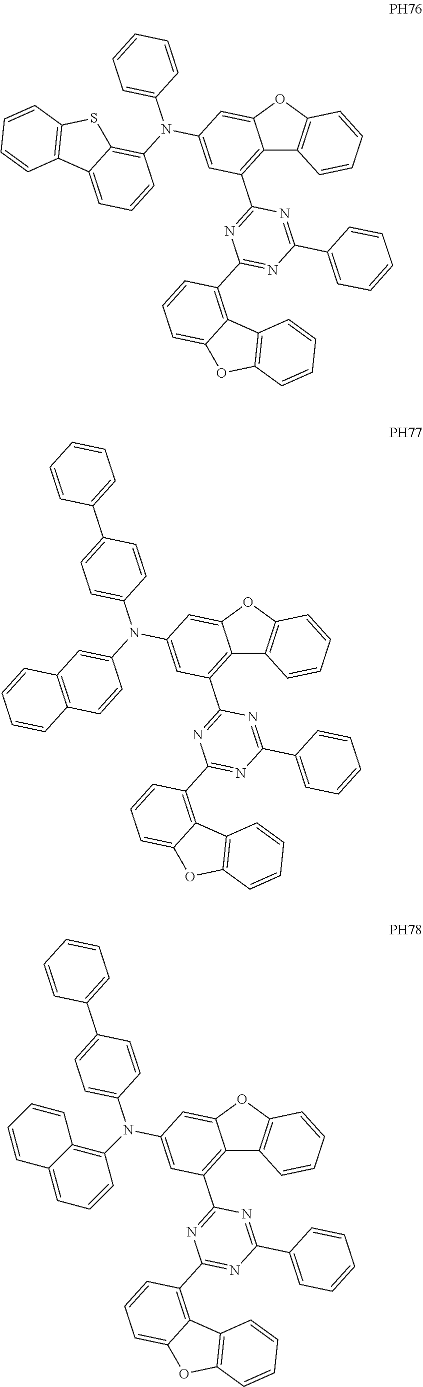 [SMILES: c1ccc(-c2nc(-c3cccc4oc5ccccc5c34)nc(-c3cc(N(c4ccccc4)c4cccc5c4sc4ccccc45)cc4oc5ccccc5c34)n2)cc1 ; c1ccc(-c2ccc(N(c3ccc4ccccc4c3)c3cc(-c4nc(-c5ccccc5)nc(-c5cccc6oc7ccccc7c56)n4)c4c(c3)oc3ccccc34)cc2)cc1 ; c1ccc(-c2ccc(N(c3cc(-c4nc(-c5ccccc5)nc(-c5cccc6oc7ccccc7c56)n4)c4c(c3)oc3ccccc34)c3cccc4ccccc34)cc2)cc1]

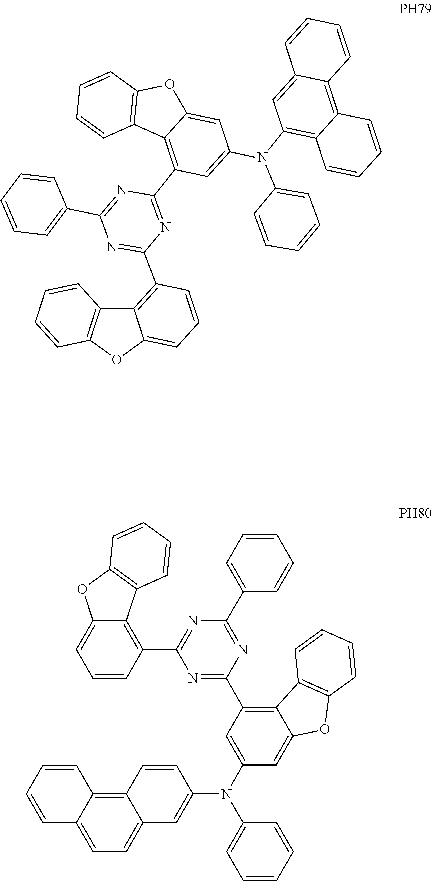 [SMILES: c1ccc(-c2nc(-c3cccc4oc5ccccc5c34)nc(-c3cc(N(c4ccccc4)c4cc5ccccc5c5ccccc45)cc4oc5ccccc5c34)n2)cc1 ; c1ccc(-c2nc(-c3cccc4oc5ccccc5c34)nc(-c3cc(N(c4ccccc4)c4ccc5c(ccc6ccccc65)c4)cc4oc5ccccc5c34)n2)cc1]

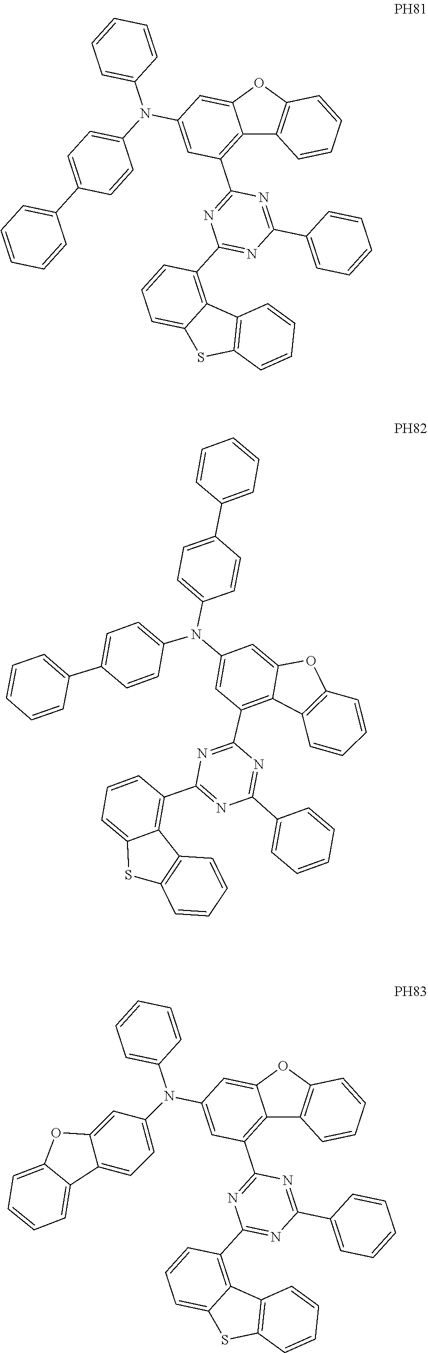 [SMILES: c1ccc(-c2ccc(N(c3ccccc3)c3cc(-c4nc(-c5ccccc5)nc(-c5cccc6sc7ccccc7c56)n4)c4c(c3)oc3ccccc34)cc2)cc1 ; c1ccc(-c2ccc(N(c3ccc(-c4ccccc4)cc3)c3cc(-c4nc(-c5ccccc5)nc(-c5cccc6sc7ccccc7c56)n4)c4c(c3)oc3ccccc34)cc2)cc1 ; c1ccc(-c2nc(-c3cc(N(c4ccccc4)c4ccc5c(c4)oc4ccccc45)cc4oc5ccccc5c34)nc(-c3cccc4sc5ccccc5c34)n2)cc1]

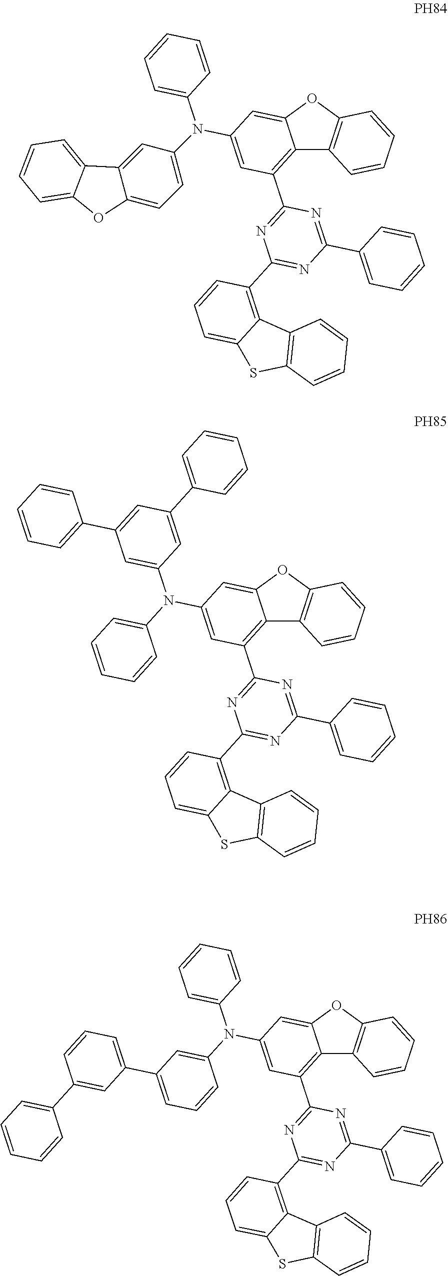 [SMILES: c1ccc(-c2nc(-c3cc(N(c4ccccc4)c4ccc5oc6ccccc6c5c4)cc4oc5ccccc5c34)nc(-c3cccc4sc5ccccc5c34)n2)cc1 ; c1ccc(-c2cc(-c3ccccc3)cc(N(c3ccccc3)c3cc(-c4nc(-c5ccccc5)nc(-c5cccc6sc7ccccc7c56)n4)c4c(c3)oc3ccccc34)c2)cc1 ; c1ccc(-c2cccc(-c3cccc(N(c4ccccc4)c4cc(-c5nc(-c6ccccc6)nc(-c6cccc7sc8ccccc8c67)n5)c5c(c4)oc4ccccc45)c3)c2)cc1]

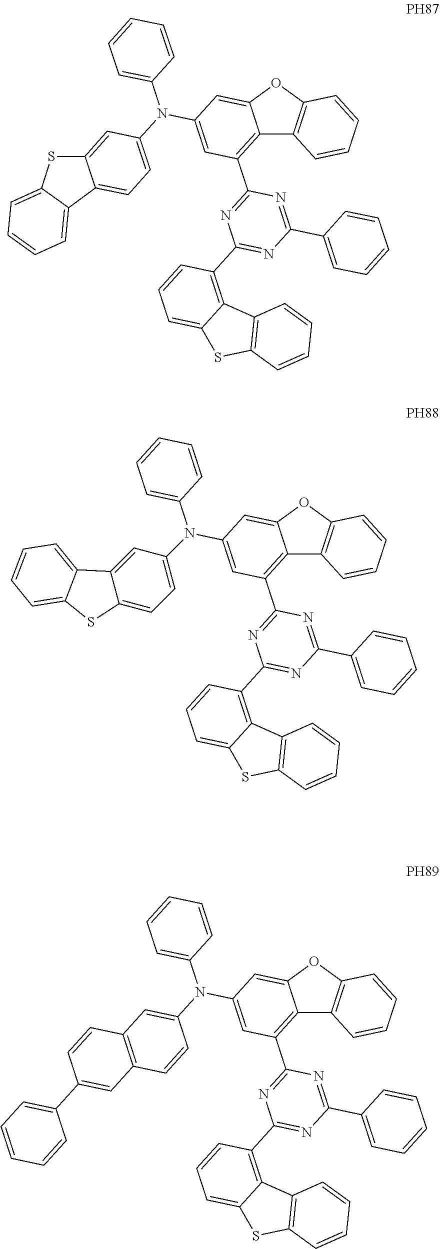 [SMILES: c1ccc(-c2nc(-c3cc(N(c4ccccc4)c4ccc5c(c4)sc4ccccc45)cc4oc5ccccc5c34)nc(-c3cccc4sc5ccccc5c34)n2)cc1 ; c1ccc(-c2nc(-c3cc(N(c4ccccc4)c4ccc5sc6ccccc6c5c4)cc4oc5ccccc5c34)nc(-c3cccc4sc5ccccc5c34)n2)cc1 ; c1ccc(-c2ccc3cc(N(c4ccccc4)c4cc(-c5nc(-c6ccccc6)nc(-c6cccc7sc8ccccc8c67)n5)c5c(c4)oc4ccccc45)ccc3c2)cc1]

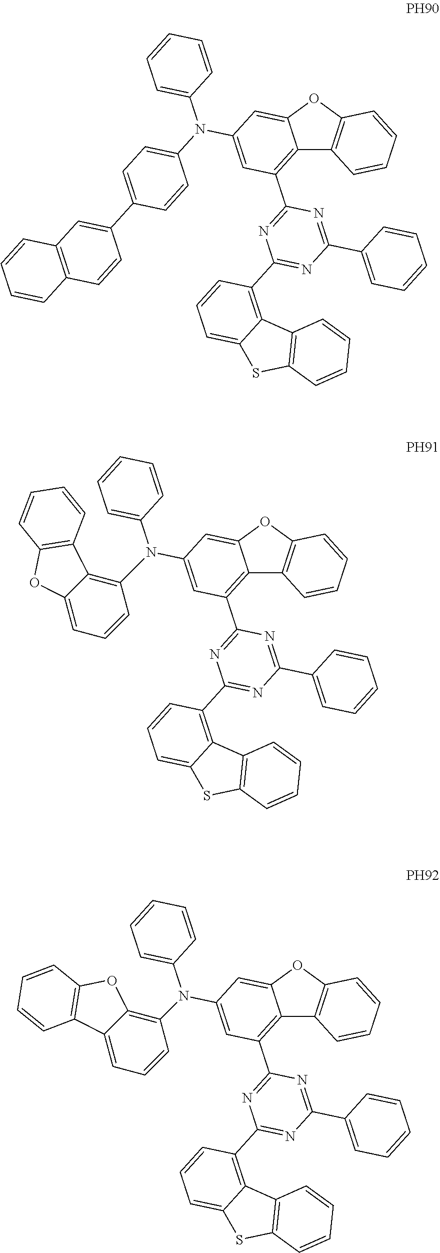 [SMILES: c1ccc(-c2nc(-c3cc(N(c4ccccc4)c4ccc(-c5ccc6ccccc6c5)cc4)cc4oc5ccccc5c34)nc(-c3cccc4sc5ccccc5c34)n2)cc1 ; c1ccc(-c2nc(-c3cc(N(c4ccccc4)c4cccc5oc6ccccc6c45)cc4oc5ccccc5c34)nc(-c3cccc4sc5ccccc5c34)n2)cc1 ; c1ccc(-c2nc(-c3cc(N(c4ccccc4)c4cccc5c4oc4ccccc45)cc4oc5ccccc5c34)nc(-c3cccc4sc5ccccc5c34)n2)cc1]

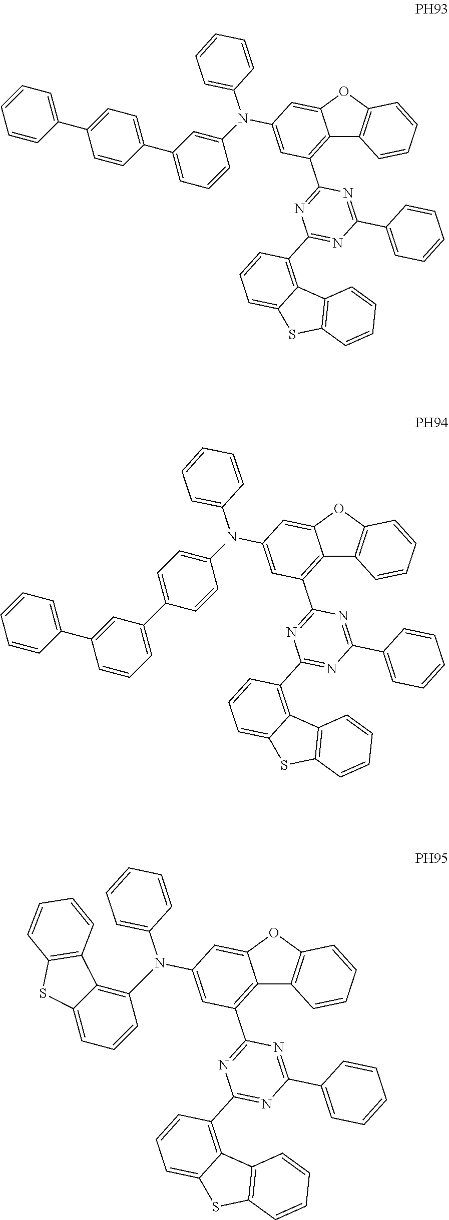 [SMILES: c1ccc(-c2ccc(-c3cccc(N(c4ccccc4)c4cc(-c5nc(-c6ccccc6)nc(-c6cccc7sc8ccccc8c67)n5)c5c(c4)oc4ccccc45)c3)cc2)cc1 ; c1ccc(-c2cccc(-c3ccc(N(c4ccccc4)c4cc(-c5nc(-c6ccccc6)nc(-c6cccc7sc8ccccc8c67)n5)c5c(c4)oc4ccccc45)cc3)c2)cc1 ; c1ccc(-c2nc(-c3cc(N(c4ccccc4)c4cccc5sc6ccccc6c45)cc4oc5ccccc5c34)nc(-c3cccc4sc5ccccc5c34)n2)cc1]

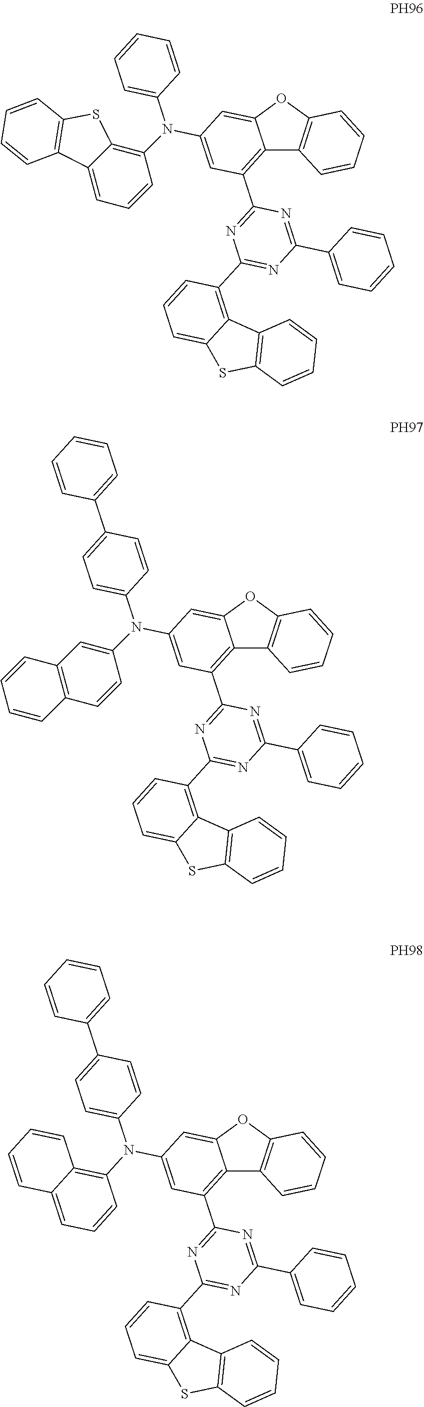 [SMILES: c1ccc(-c2nc(-c3cc(N(c4ccccc4)c4cccc5c4sc4ccccc45)cc4oc5ccccc5c34)nc(-c3cccc4sc5ccccc5c34)n2)cc1 ; c1ccc(-c2ccc(N(c3ccc4ccccc4c3)c3cc(-c4nc(-c5ccccc5)nc(-c5cccc6sc7ccccc7c56)n4)c4c(c3)oc3ccccc34)cc2)cc1 ; c1ccc(-c2ccc(N(c3cc(-c4nc(-c5ccccc5)nc(-c5cccc6sc7ccccc7c56)n4)c4c(c3)oc3ccccc34)c3cccc4ccccc34)cc2)cc1]

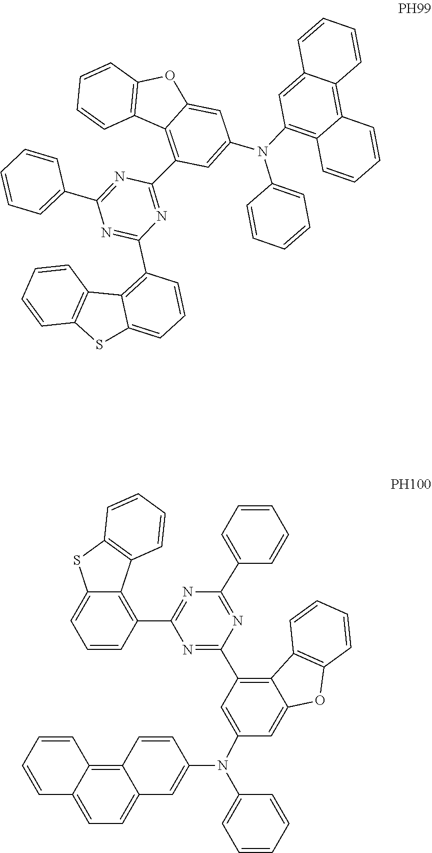 [SMILES: c1ccc(-c2nc(-c3cc(N(c4ccccc4)c4cc5ccccc5c5ccccc45)cc4oc5ccccc5c34)nc(-c3cccc4sc5ccccc5c34)n2)cc1 ; c1ccc(-c2nc(-c3cc(N(c4ccccc4)c4ccc5c(ccc6ccccc65)c4)cc4oc5ccccc5c34)nc(-c3cccc4sc5ccccc5c34)n2)cc1]

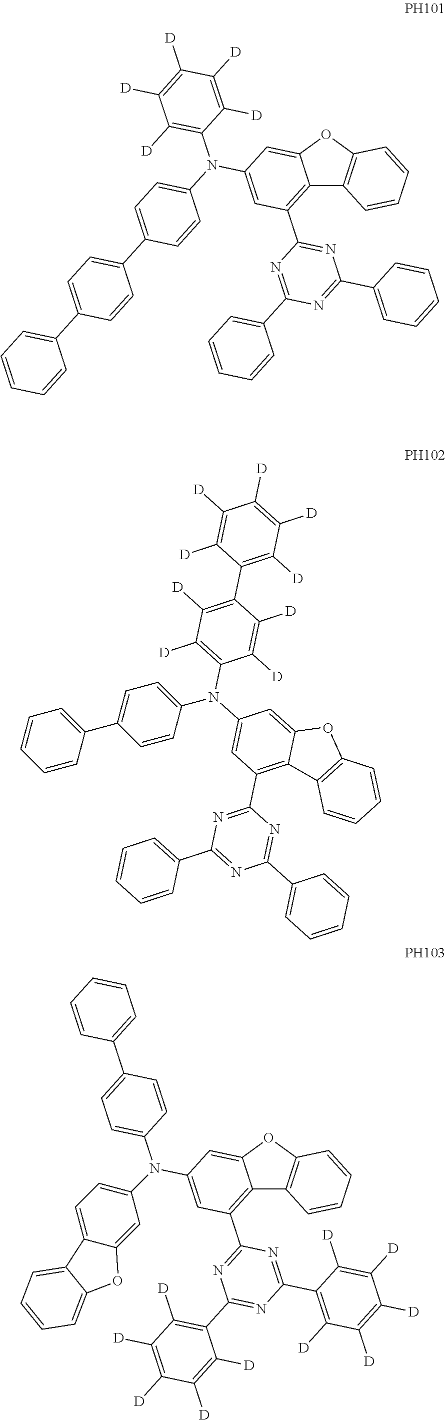 [SMILES: [2H]c1c([2H])c([2H])c(N(c2ccc(-c3ccc(-c4ccccc4)cc3)cc2)c2cc(-c3nc(-c4ccccc4)nc(-c4ccccc4)n3)c3c(c2)oc2ccccc23)c([2H])c1[2H] ; [2H]c1c([2H])c([2H])c(-c2c([2H])c([2H])c(N(c3ccc(-c4ccccc4)cc3)c3cc(-c4nc(-c5ccccc5)nc(-c5ccccc5)n4)c4c(c3)oc3ccccc34)c([2H])c2[2H])c([2H])c1[2H] ; [2H]c1c([2H])c([2H])c(-c2nc(-c3c([2H])c([2H])c([2H])c([2H])c3[2H])nc(-c3cc(N(c4ccc(-c5ccccc5)cc4)c4ccc5c(c4)oc4ccccc45)cc4oc5ccccc5c34)n2)c([2H])c1[2H]]

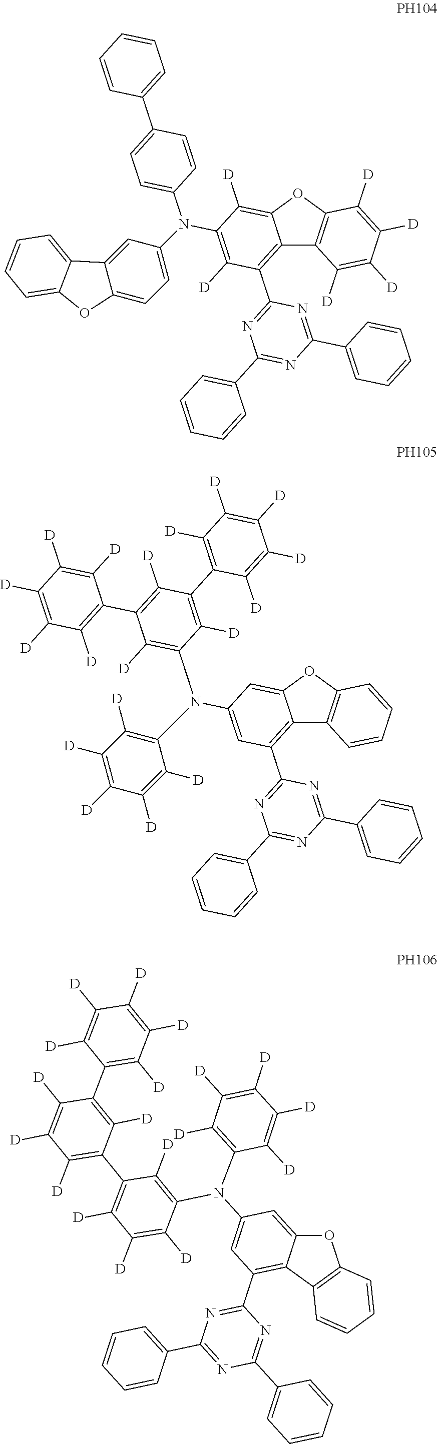

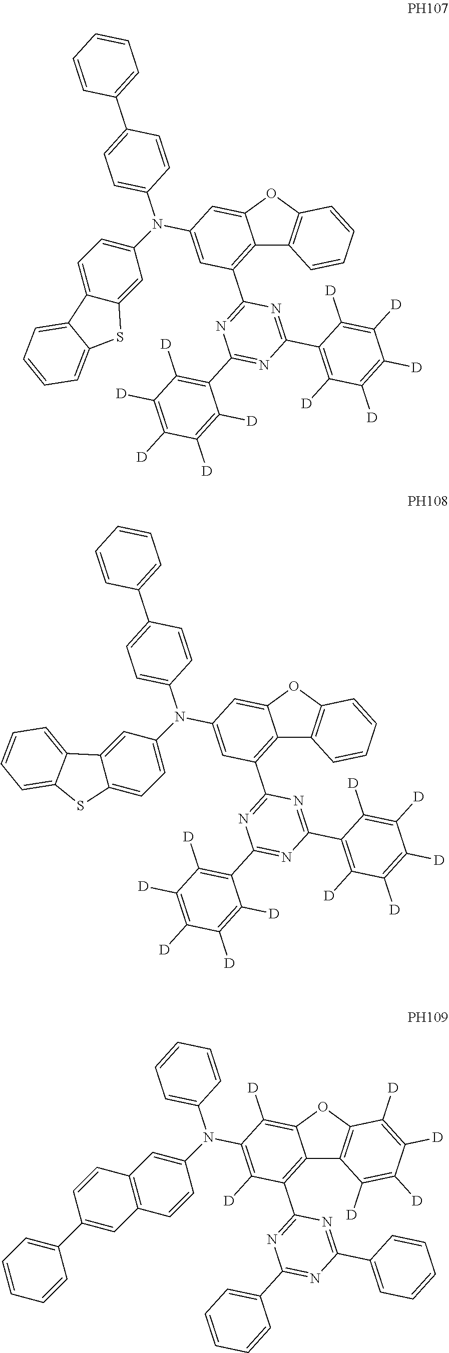 [SMILES: [2H]c1c([2H])c([2H])c(-c2nc(-c3c([2H])c([2H])c([2H])c([2H])c3[2H])nc(-c3cc(N(c4ccc(-c5ccccc5)cc4)c4ccc5c(c4)sc4ccccc45)cc4oc5ccccc5c34)n2)c([2H])c1[2H] ; [2H]c1c([2H])c([2H])c(-c2nc(-c3c([2H])c([2H])c([2H])c([2H])c3[2H])nc(-c3cc(N(c4ccc(-c5ccccc5)cc4)c4ccc5sc6ccccc6c5c4)cc4oc5ccccc5c34)n2)c([2H])c1[2H] ; [2H]c1c([2H])c([2H])c2c(oc3c([2H])c(N(c4ccccc4)c4ccc5cc(-c6ccccc6)ccc5c4)c([2H])c(-c4nc(-c5ccccc5)nc(-c5ccccc5)n4)c32)c1[2H]]

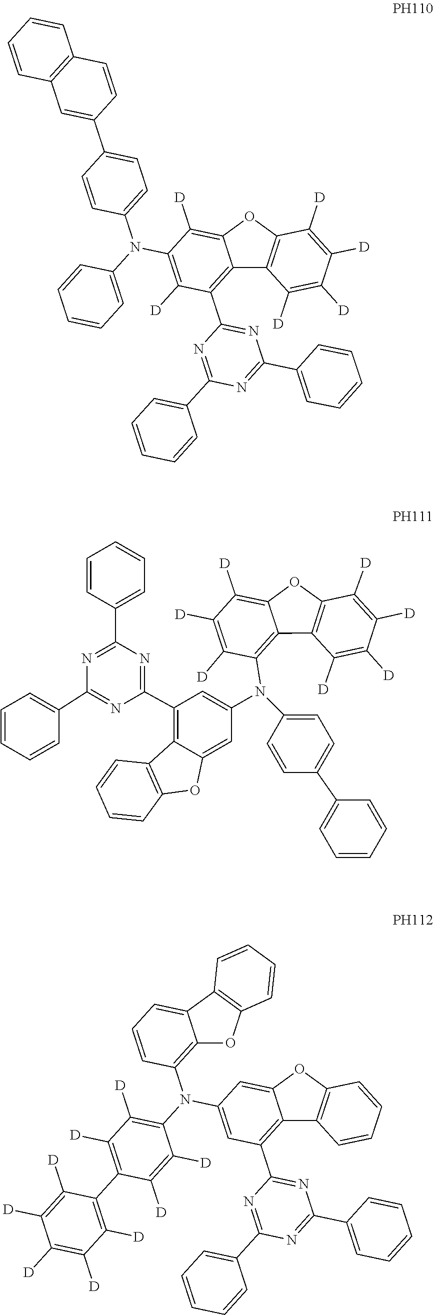 [SMILES: [2H]c1c([2H])c([2H])c2c(oc3c([2H])c(N(c4ccccc4)c4ccc(-c5ccc6ccccc6c5)cc4)c([2H])c(-c4nc(-c5ccccc5)nc(-c5ccccc5)n4)c32)c1[2H] ; [2H]c1c([2H])c([2H])c2c(oc3c([2H])c([2H])c([2H])c(N(c4ccc(-c5ccccc5)cc4)c4cc(-c5nc(-c6ccccc6)nc(-c6ccccc6)n5)c5c(c4)oc4ccccc45)c32)c1[2H] ; [2H]c1c([2H])c([2H])c(-c2c([2H])c([2H])c(N(c3cc(-c4nc(-c5ccccc5)nc(-c5ccccc5)n4)c4c(c3)oc3ccccc34)c3cccc4c3oc3ccccc34)c([2H])c2[2H])c([2H])c1[2H]]

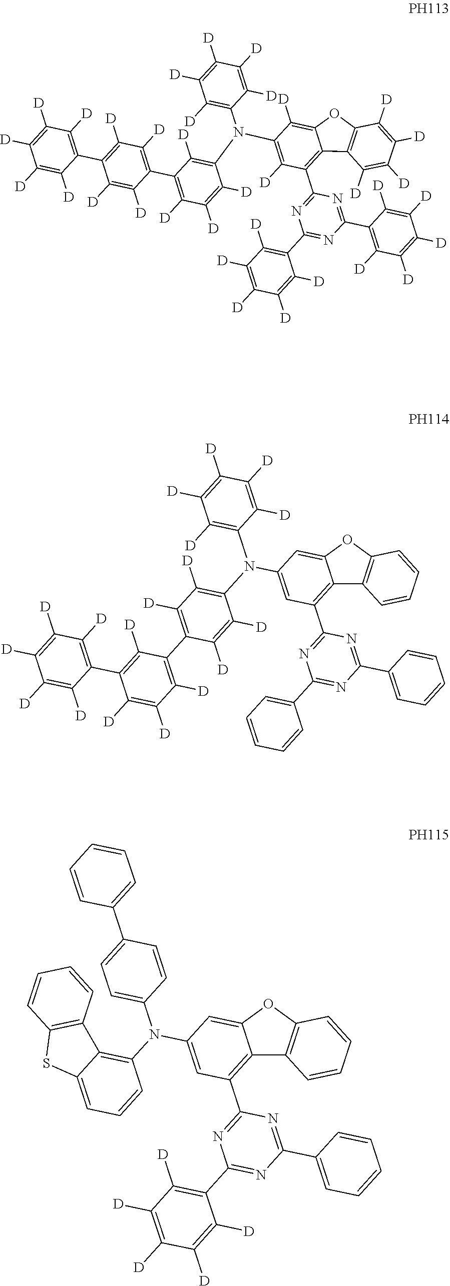

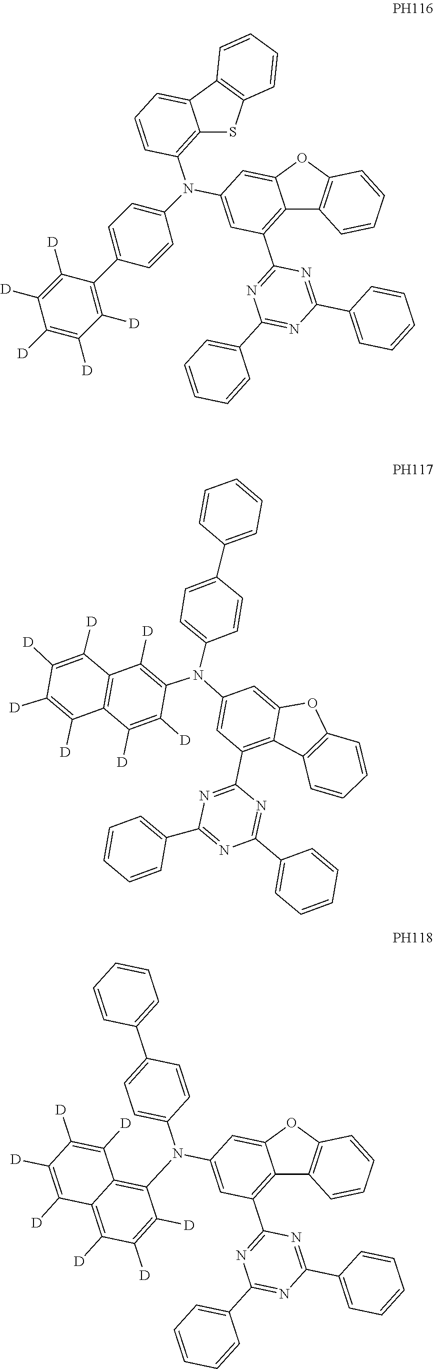 [SMILES: [2H]c1c([2H])c([2H])c(-c2ccc(N(c3cc(-c4nc(-c5ccccc5)nc(-c5ccccc5)n4)c4c(c3)oc3ccccc34)c3cccc4c3sc3ccccc34)cc2)c([2H])c1[2H] ; [2H]c1c([2H])c([2H])c2c([2H])c(N(c3ccc(-c4ccccc4)cc3)c3cc(-c4nc(-c5ccccc5)nc(-c5ccccc5)n4)c4c(c3)oc3ccccc34)c([2H])c([2H])c2c1[2H] ; [2H]c1c([2H])c([2H])c2c(N(c3ccc(-c4ccccc4)cc3)c3cc(-c4nc(-c5ccccc5)nc(-c5ccccc5)n4)c4c(c3)oc3ccccc34)c([2H])c([2H])c([2H])c2c1[2H]]

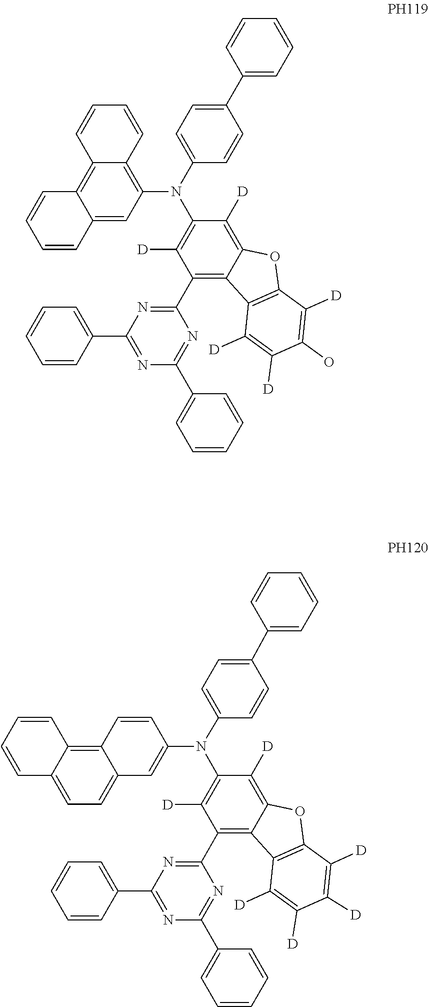

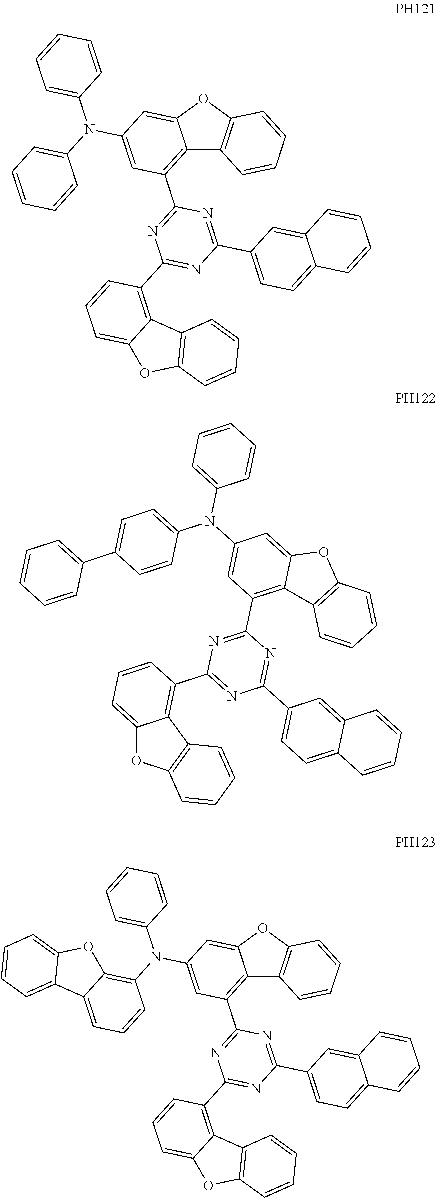

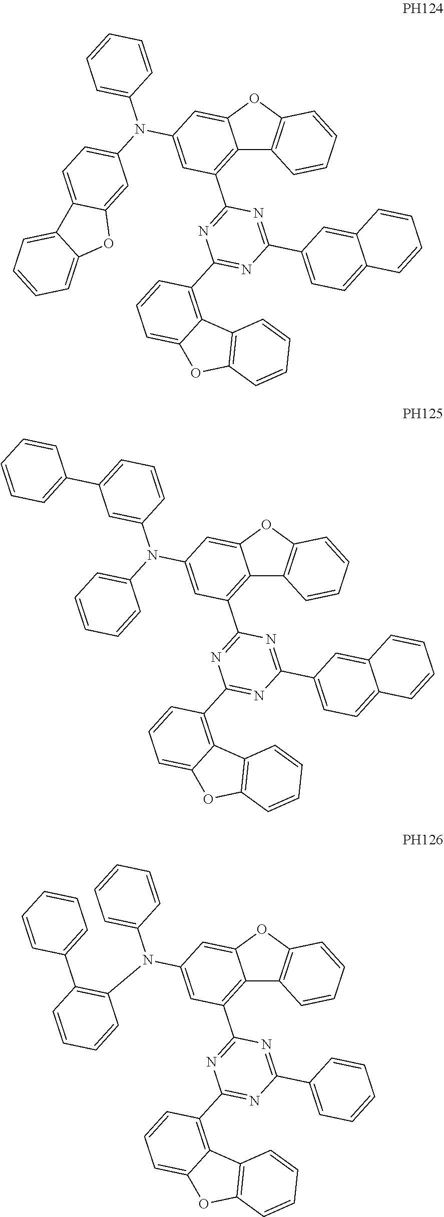 [SMILES: c1ccc(N(c2ccc3c(c2)oc2ccccc23)c2cc(-c3nc(-c4ccc5ccccc5c4)nc(-c4cccc5oc6ccccc6c45)n3)c3c(c2)oc2ccccc23)cc1 ; c1ccc(-c2cccc(N(c3ccccc3)c3cc(-c4nc(-c5ccc6ccccc6c5)nc(-c5cccc6oc7ccccc7c56)n4)c4c(c3)oc3ccccc34)c2)cc1 ; c1ccc(-c2nc(-c3cccc4oc5ccccc5c34)nc(-c3cc(N(c4ccccc4)c4ccccc4-c4ccccc4)cc4oc5ccccc5c34)n2)cc1]

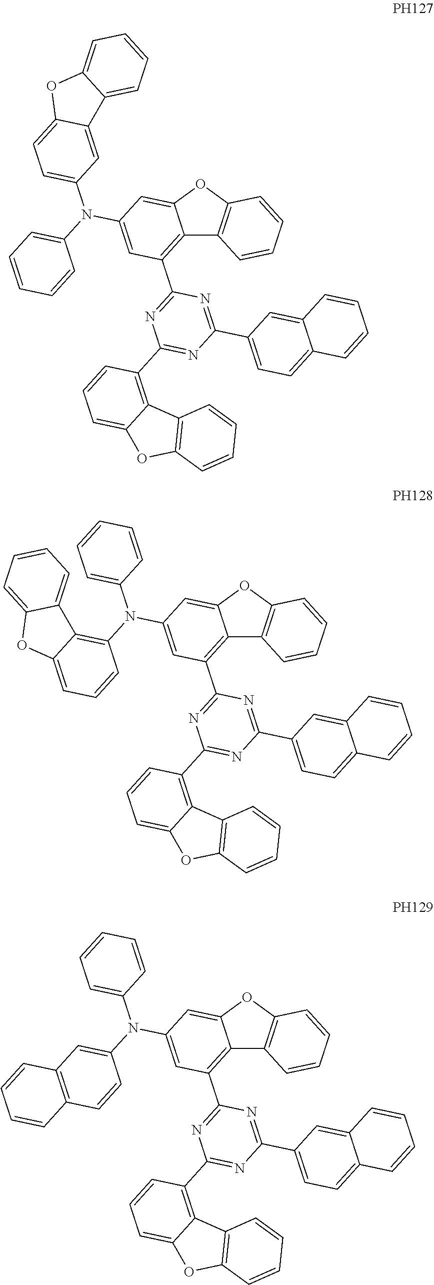 [SMILES: c1ccc(N(c2cc(-c3nc(-c4ccc5ccccc5c4)nc(-c4cccc5oc6ccccc6c45)n3)c3c(c2)oc2ccccc23)c2ccc3oc4ccccc4c3c2)cc1 ; c1ccc(N(c2cc(-c3nc(-c4ccc5ccccc5c4)nc(-c4cccc5oc6ccccc6c45)n3)c3c(c2)oc2ccccc23)c2cccc3oc4ccccc4c23)cc1 ; c1ccc(N(c2ccc3ccccc3c2)c2cc(-c3nc(-c4ccc5ccccc5c4)nc(-c4cccc5oc6ccccc6c45)n3)c3c(c2)oc2ccccc23)cc1]

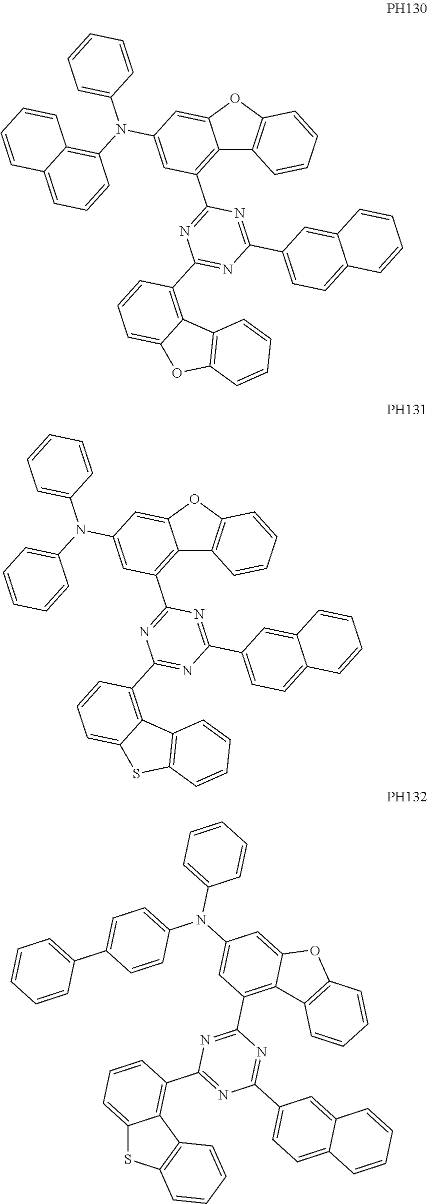

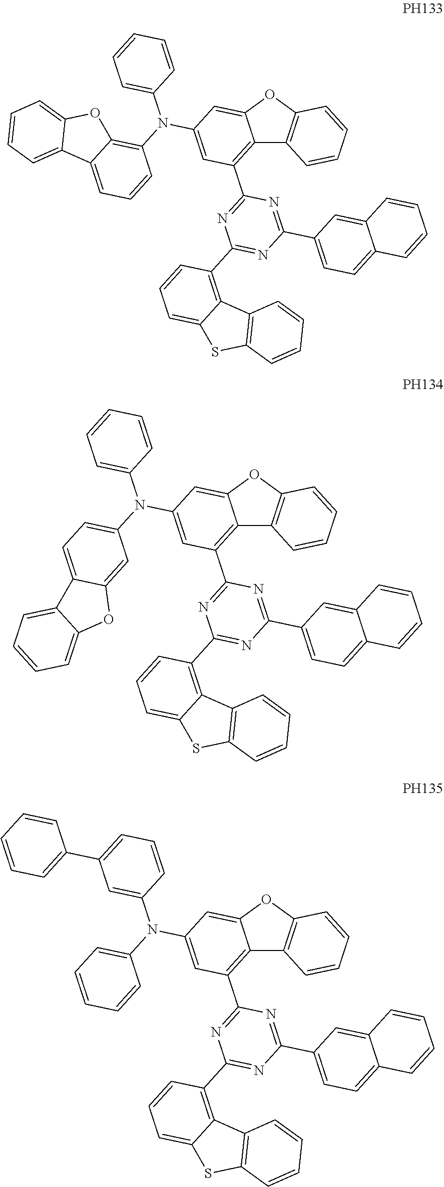 [SMILES: c1ccc(N(c2cc(-c3nc(-c4ccc5ccccc5c4)nc(-c4cccc5sc6ccccc6c45)n3)c3c(c2)oc2ccccc23)c2cccc3c2oc2ccccc23)cc1 ; c1ccc(N(c2ccc3c(c2)oc2ccccc23)c2cc(-c3nc(-c4ccc5ccccc5c4)nc(-c4cccc5sc6ccccc6c45)n3)c3c(c2)oc2ccccc23)cc1 ; c1ccc(-c2cccc(N(c3ccccc3)c3cc(-c4nc(-c5ccc6ccccc6c5)nc(-c5cccc6sc7ccccc7c56)n4)c4c(c3)oc3ccccc34)c2)cc1]

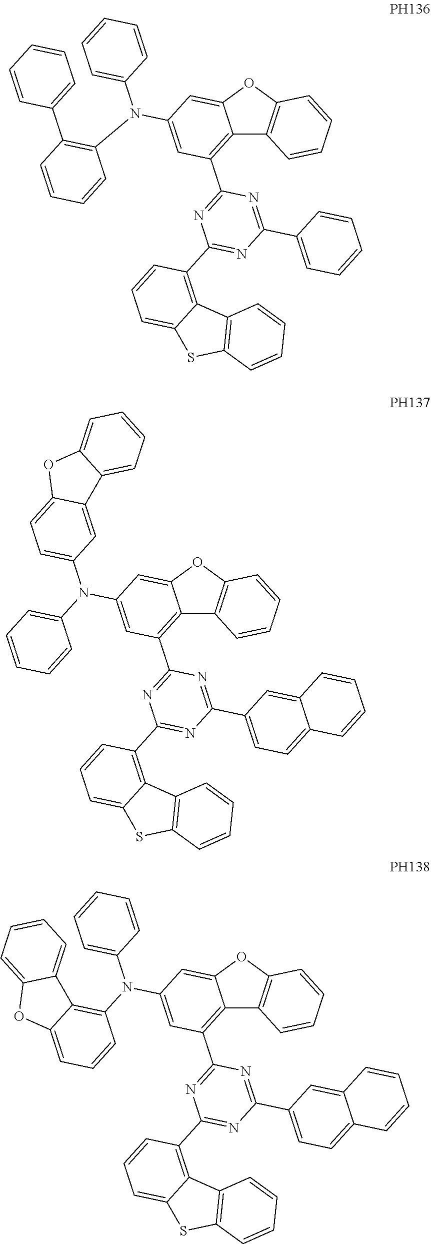 [SMILES: c1ccc(-c2nc(-c3cc(N(c4ccccc4)c4ccccc4-c4ccccc4)cc4oc5ccccc5c34)nc(-c3cccc4sc5ccccc5c34)n2)cc1 ; c1ccc(N(c2cc(-c3nc(-c4ccc5ccccc5c4)nc(-c4cccc5sc6ccccc6c45)n3)c3c(c2)oc2ccccc23)c2ccc3oc4ccccc4c3c2)cc1 ; c1ccc(N(c2cc(-c3nc(-c4ccc5ccccc5c4)nc(-c4cccc5sc6ccccc6c45)n3)c3c(c2)oc2ccccc23)c2cccc3oc4ccccc4c23)cc1]

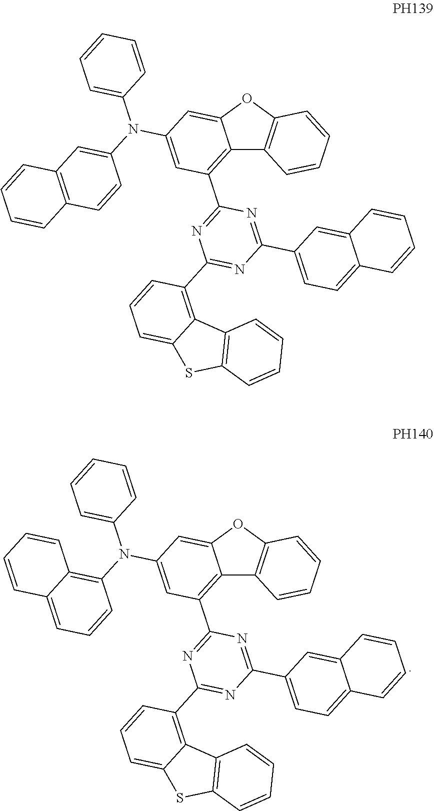 [SMILES: c1ccc(N(c2ccc3ccccc3c2)c2cc(-c3nc(-c4ccc5ccccc5c4)nc(-c4cccc5sc6ccccc6c45)n3)c3c(c2)oc2ccccc23)cc1 ; c1ccc(N(c2cc(-c3nc(-c4ccc5ccccc5c4)nc(-c4cccc5sc6ccccc6c45)n3)c3c(c2)oc2ccccc23)c2cccc3ccccc23)cc1]

12. The organic light emitting device of claim 8, wherein the organic material layer comprises a light emitting layer, and the light emitting layer comprises the heterocyclic compound represented by Chemical Formula 1.

13. The organic light emitting device of claim 8, wherein the organic material layer comprises a light emitting layer, the light emitting layer comprises a host material, and the host material comprises the heterocyclic compound represented by Chemical Formula 1.

14. The organic light emitting device of claim 8, further comprising one or two or more layers selected from the group consisting of a light emitting layer, a hole injection layer, a hole transport layer, an electron blocking layer, a hole blocking layer, an electron transport layer and an electron injection layer.

15. A composition for an organic material layer of an organic light emitting device, comprising the heterocyclic compound represented by Chemical Formula 1 according to claim 1; and a heterocyclic compound represented by the following Chemical Formula 2: 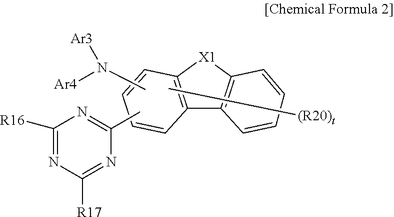
in Chemical Formula 2,
X1 is O or S,
R20 is hydrogen; deuterium; a halogen; a cyano group; a substituted or unsubstituted C1 to C60 alkyl group; a substituted or unsubstituted C6 to C60 aryl group; or a substituted or unsubstituted C2 to C60 heteroaryl group,
t is an integer from 1 to 4, and when t is 2 or higher, substituents in the parenthesis are the same as or different from each other,
Ar3 and Ar4 are the same as or different from each other, and are each independently a substituted or unsubstituted C6 to C60 aryl group; or a substituted or unsubstituted C2 to C60 heteroaryl group, and
R16 and R17 are the same as or different from each other, and are each independently a substituted or unsubstituted C6 to C60 aryl group; or a substituted or unsubstituted C2 to C60 heteroaryl group.

16. The composition of claim 15, wherein a weight ratio of the heterocyclic compound represented by Chemical Formula 1: the heterocyclic compound represented by Chemical Formula 2 in the composition is 1:10 to 10:1.
